(19) United States
(12) Patent Application Publication
(10) Pub. No.: US 2025/0152726 A1
(43) Pub. Date: May 15, 2025

(54) NUCLEIC ACID-POLYPEPTIDE COMPOSITIONS AND USES THEREOF

(71) Applicant: Avidity Biosciences, Inc., San Diego, CA (US)

(72) Inventors: Andrew John GEALL, Carlsbad, CA (US); Venkata Ramana DOPPALAPUDI, San Diego, CA (US); Joel Daniel ARIAS, San Diego, CA (US); David Sai-Ho CHU, San Diego, CA (US); Michael Caramian COCHRAN, La Jolla, CA (US); Rob BURKE, Encinitas, CA (US); Philip KOVACH, San Diego, CA (US); Barbora MALECOVA, San Diego, CA (US)

(21) Appl. No.: 19/031,264

(22) Filed: Jan. 17, 2025

Related U.S. Application Data
(62) Division of application No. 17/364,765, filed on Jun. 30, 2021.
Application No. 17/364,765 is a division of application No. 16/152,324, filed on Oct. 4, 2018, now Pat. No. 11,110,180.
(60) Provisional application No. 62/568,238, filed on Oct. 4, 2017.

(51) Int. Cl. A61K 47/68; A61K 31/713; A61K 47/60; A61K 47/64; A61P 35/00; C07H 17/00; C07K 16/28; C12N 15/113

(52) U.S. Cl. CPC A61K 47/6807; A61K 31/713; A61K 47/60; A61K 47/64; A61K 47/6811; A61K 47/6849; A61K 47/6883; A61P 35/00; C07H 17/00; C07K 16/2863; C12N 15/113; C12N 2310/312; C12N 2310/3125; C12N 2310/315; C12N 2310/3513; C12N 2310/3515

(57) ABSTRACT

Disclosed herein are compositions and pharmaceutical formulations that comprise a binding moiety conjugated to a modified polynucleic acid molecule and a polymer. Also described herein include methods for treating a cancer which utilize a composition or a pharmaceutical formulation comprising a binding moiety conjugated to a polynucleic acid molecule and a polymer.

### CROSS-REFERENCE

This application is a divisional of U.S. patent application Ser. No. 17/364,765, filed Jun. 30, 2021, which is a divisional of U.S. patent application Ser. No. 16/152,324, filed on Oct. 4, 2018, which claims the benefit of U.S. Provisional Patent Application No. 62/568,238, filed on Oct. 4, 2017, which is incorporated herein by reference in its entirety.

### SEQUENCE LISTING

The instant application contains a Sequence Listing which has been submitted electronically in XML format and is hereby incorporated by reference in its entirety. Said XML copy, created on Jan. 17, 2025, is named 45532-719 403 SL.xml and is 1,704,090 bytes bytes in size.

### BACKGROUND OF THE DISCLOSURE

Gene suppression by RNA-induced gene silencing provides several levels of control: transcription inactivation, small interfering RNA (siRNA)-induced mRNA degradation, and siRNA-induced transcriptional attenuation. In some instances, RNA interference (RNAi) provides long lasting effect over multiple cell divisions. As such, RNAi represents a viable method useful for drug target validation, gene function analysis, pathway analysis, and disease therapeutics.

### SUMMARY OF THE DISCLOSURE

Disclosed herein, in certain embodiments, are compositions and pharmaceutical formulations that comprise a binding moiety conjugated to a polynucleic acid molecule and a polymer. In some embodiments, also described herein include methods for treating a disease or condition (e.g., cancer) that utilize a composition or a pharmaceutical formulation comprising a binding moiety conjugated to a polynucleic acid molecule and a polymer.

Disclosed herein, in certain embodiments, is a molecule of Formula (I):

A-X—B—Y—C Formula I

wherein,
A is a binding moiety;
B is a polynucleotide;
C is a polymer;
X is a bond or a first linker; and
Y is a bond or a second linker; and

wherein the polynucleotide comprises at least one 5′-vinylphosphonate modified nucleotide.

In some embodiments, the polynucleotide comprises a single strand. In some embodiments, the polynucleotide comprises two or more strands. In some embodiments, the polynucleotide comprises a first polynucleotide and a second polynucleotide hybridized to the first polynucleotide to form a double-stranded polynucleic acid molecule. In some embodiments, the second polynucleotide comprises at least one modification.

In some embodiments, the first polynucleotide and the second polynucleotide are RNA molecules. In some embodiments, the first polynucleotide and the second polynucleotide are siRNA molecules.

In some embodiments, the first polynucleotide comprises a sequence having at least 60%, 65%, 70%, 75%, 80%, 85%, 90%, 95%, 96%, 97%, 98%, 99%, or 100% sequence identity to SEQ ID NOs: 16-45, 422-1173, 1195-1214, or 1215-1242. In some embodiments, the first polynucleotide consists of a sequence selected from SEQ ID NOs: 16-45, 422-1173, 1195-1214, or 1215-1242.

In some embodiments, the second polynucleotide comprises a sequence having at least 60%, 65%, 70%, 75%, 80%, 85%, 90%, 95%, 96%, 97%, 98%, 99%, or 100% sequence identity to SEQ ID NOs: 16-45, 422-1173, 1195-1214, or 1215-1242. In some embodiments, the second polynucleotide consists of a sequence selected from SEQ ID NOs: 16-45, 422-1173, 1195-1214, or 1215-1242.

In some embodiments, X and Y are independently a bond or a non-polymeric linker group. In some embodiments, X is a bond. In some embodiments, X is a C₁-C₆ alkyl group. In some embodiments, Y is a C₁-C₆ alkyl group. In some embodiments, X is a homobifuctional linker or a heterobifunctional linker, optionally conjugated to a C₁-C₆ alkyl group. In some embodiments, Y is a homobifuctional linker or a heterobifunctional linker.

In some embodiments, the binding moiety is an antibody or binding fragment thereof. In some embodiments, the antibody or binding fragment thereof comprises a humanized antibody or binding fragment thereof, chimeric antibody or binding fragment thereof, monoclonal antibody or binding fragment thereof, monovalent Fab′, divalent Fab2, single-chain variable fragment (scFv), diabody, minibody, nanobody, single-domain antibody (sdAb), or camelid antibody or binding fragment thereof. In some embodiments, the antibody or binding fragment thereof is an anti-EGFR antibody or binding fragment thereof.

In some embodiments, C is polyethylene glycol. In some embodiments, C has a molecular weight of about 5000 Da.

In some embodiments, A-X is conjugated to the 5′ end of B and Y—C is conjugated to the 3′ end of B. In some embodiments, Y—C is conjugated to the 5′ end of B and A-X is conjugated to the 3′ end of B. In some embodiments, A-X, Y—C or a combination thereof is conjugated to an internucleotide linkage group.

In some embodiments, the molecule further comprises D. In some embodiments, D is conjugated to C or to A.

In some embodiments, D is conjugated to the molecule of Formula (I) according to Formula (II):

(A-X—B—Y—Cₙ)-L-D Formula II

wherein,
A is a binding moiety;
B is a polynucleotide;
C is a polymer;
X is a bond or a first linker;
Y is a bond or a second linker;
L is a bond or a third linker;
D is an endosomolytic moiety; and
n is an integer between 0 and 1; and

wherein the polynucleotide comprises at least one 5′-vinylphosphonate modified nucleotide; and

D is conjugated anywhere on A, B, or C.

In some embodiments, D is INF7 or melittin.

In some embodiments, D is an endosomolytic polymer.

In some embodiments, L is a C₁-C₆ alkyl group. In some embodiments, L is a homobifuctional linker or a heterobifunctional linker.

In some embodiments, the molecule further comprises at least a second binding moiety A. In some embodiments, the at least second binding moiety A is conjugated to A, to B, or to C. In some embodiments, the at least second binding moiety A is cholesterol.

In some embodiments, the molecule further comprises at least an additional polynucleotide B. In some embodiments, the at least an additional polynucleotide B is conjugated to A, to B, or to C.

In some embodiments, the molecule further comprises at least an additional polymer C. In some embodiments, the at least an additional polymer C is conjugated to A, to B, or to C.

Disclosed herein, in certain embodiments, is a molecule of Formula (I): A-X—B—Y—C(Formula I), wherein A is an antibody or its binding fragments thereof; B is a polynucleotide; C is a polymer; X is a bond or first non-polymeric linker; and Y is a bond or second linker; wherein the polynucleotide comprises at least one 5′-vinylphosphonate modified nucleotide; and wherein A and C are not attached to B at the same terminus. In some embodiments, the at least one modified internucleotide linkage comprises a phosphorothioate linkage or a phosphorodithioate linkage. In some embodiments, the at least one inverted abasic moiety is at at least one terminus. In some embodiments, the polynucleotide comprises a single strand. In some embodiments, the polynucleotide comprises a first polynucleotide and a second polynucleotide hybridized to the first polynucleotide to form a double-stranded polynucleic acid molecule. In some embodiments, the second polynucleotide comprises at least one modification. In some embodiments, the first polynucleotide and the second polynucleotide are RNA molecules. In some embodiments, the first polynucleotide comprises a sequence having at least 80%, 85%, 90%, 95%, 96%, 97%, 98%, 99%, or 100% sequence identity to SEQ ID NOs: 16-45, 422-1173, 1195-1214, or 1215-1242. In some embodiments, the second polynucleotide comprises a sequence having at least 80%, 85%, 90%, 95%, 96%, 97%, 98%, 99%, or 100% sequence identity to SEQ ID NOs: 16-45, 422-1173, 1195-1214, or 1215-1242. In some embodiments, Y is a non-polymeric linker group. In some embodiments, X is a bond. In some embodiments, X is a C₁-C₆ alkyl group. In some embodiments, Y is a C₁-C₆ alkyl group. In some embodiments, X is a homobifuctional linker or a heterobifunctional linker, optionally conjugated to a C₁-C₆ alkyl group. In some embodiments, Y is a homobifuctional linker or a heterobifunctional linker. In some embodiments, the antibody or binding fragment thereof comprises a humanized antibody or binding fragment thereof, chimeric antibody or binding fragment thereof, monoclonal antibody or binding fragment thereof, monovalent Fab′, divalent Fab2, single-chain variable fragment (scFv), diabody, minibody, nanobody, single-domain antibody (sdAb), or camelid antibody or binding fragment thereof. In some embodiments, C is polyethylene glycol. In some embodiments, C has a molecular weight of about 1000 Da, 2000 Da, or 5000 Da. In some embodiments, A-X is conjugated to the 5′ end of B and Y—C is conjugated to the 3′ end of B. In some embodiments, Y—C is conjugated to the 5′ end of B and A-X is conjugated to the 3′ end of B. In some embodiments, the molecule further comprises D. In some embodiments, D is conjugated to C or to A. In some embodiments, D is conjugated to the molecule of Formula (I) according to Formula (II): (A-X—B—Y—C_{c})-L-D (Formula II), wherein A is an antibody or its binding fragments thereof; B is a polynucleotide; C is a polymer; X is a bond or first non-polymeric linker; Y is a bond or second linker; L is a bond or third linker; D is an endosomolytic moiety; and c is an integer between 0 and 1; wherein the polynucleotide comprises at least one 2′ modified nucleotide, at least one modified internucleotide linkage, or at least one inverted abasic moiety; wherein A and C are not attached to B at the same terminus; and wherein D is conjugated anywhere on A or C or to a terminus of B. In some embodiments, D is INF7 or melittin. In some embodiments, D is an endosomolytic polymer. In some embodiments, L is a C₁-C₆ alkyl group. In some embodiments, L is a homobifuctional linker or a heterobifunctional linker. In some embodiments, the molecule further comprises at least a second binding moiety. In some embodiments, the at least second binding moiety is conjugated to A, to B, or to C. In some embodiments, the at least second binding moiety is cholesterol. In some embodiments, the molecule further comprises at least an additional polynucleotide B. In some embodiments, the at least an additional polynucleotide B is conjugated to A, to B, or to C. In some embodiments, the molecule further comprises at least an additional polymer C. In some embodiments, the at least an additional polymer C is conjugated to A, to B, or to C.

Disclosed herein, in certain embodiments, is a pharmaceutical composition comprising a molecule described above, and a pharmaceutically acceptable excipient. In some embodiments, the pharmaceutical composition is formulated as a nanoparticle formulation. In some embodiments, the pharmaceutical composition is formulated for parenteral, oral, intranasal, buccal, rectal, or transdermal administration.

Disclosed herein, in certain embodiments, is a method of treating a disease or disorder in a patient in need thereof, comprising administering to the patient a composition comprising a molecule described above. In some embodiments, the disease or disorder is a cancer. In some embodiments, the cancer is a solid tumor. In some embodiments, the cancer is a hematologic malignancy. In some embodiments, the cancer comprises a KRAS-associated, an EGFR-associated, an AR-associated cancer, a β-catenin associated cancer, a PIK3C-associated cancer, or a MYC-associated cancer. In some embodiments, the cancer comprises bladder cancer, breast cancer, colorectal cancer, endometrial cancer, esophageal cancer, glioblastoma multiforme, head and neck cancer, kidney cancer, lung cancer, ovarian cancer, pancreatic cancer, prostate cancer, or thyroid cancer. In some embodiments, the cancer comprises acute myeloid leukemia, CLL, DLBCL, or multiple myeloma. In some embodiments, the method is an immuno-oncology therapy.

Disclosed herein, in certain embodiments, is a method of inhibiting the expression of a target gene in a primary cell of a patient, comprising administering a molecule described above to the primary cell. In some embodiments, the method is an in vivo method. In some embodiments, the patient is a human.

Disclosed herein, in certain embodiments, is an immuno-oncology therapy comprising a molecule described above for the treatment of a disease or disorder in a patient in need thereof.

Disclosed herein, in certain embodiments, is a kit comprising a molecule described above.

### BRIEF DESCRIPTION OF THE DRAWINGS

FIG. 1A shows a cartoon representation of the structure of a 21mer duplex with 19 bases of complementarity and 3′ dinucleotide overhangs, as described in example 10.

FIG. 1B shows a cartoon representation of the structure of a blunt ended duplex with 19 bases of complementarity and one 3′ dinucleotide overhang, as described in molecular biology example 10.

FIG. 2 shows a plot of Log (siRNA in nM) vs relative HPRT mRNA (%) for HCT Transfection with HPRT siRNA as described in example 11

FIG. 3 shows a plot of Log (siRNA in nM) vs relative MSTN mRNA levels (% of untreated control) for SJCRH30 transfection with MSTN siRNAs as described in molecular biology example 12.

FIG. 4 shows in vivo MSTN and HPRT mRNA downregulation in gastroc muscle, after IV administration of antibody siRNA conjugates, as described in molecular biology example 13.

FIG. 5 shows in vivo MSTN mRNA downregulation in (A) gastroc, (B) quad and (C) heart muscle after IV administration of antibody siRNA conjugates, as described in molecular biology example 14.

FIG. 6 shows in vivo HPRT mRNA downregulation in (A) gastroc and (B) liver after IV administration of antibody siRNA conjugates, as described in molecular biology example 15.

FIG. 7 shows a plot of concentration (nM) vs % SSB mRNA as described in molecular biology example 16.

FIGS. 8A-8B shows plots of siRNA concentration (nM) vs relative MSTN (FIG. 8A) and SSB (FIG. 8B) mRNA expression for SJCRH30 transfection, as described in molecular biology example 17.

FIGS. 9A-9B shows plots of siRNA concentration (nM) vs relative MSTN (FIG. 9A) and SSB (FIG. 9B) mRNA expression DM1Ctrl Myoblasts, as described in molecular biology example 18.

FIGS. 10A-10S illustrate cartoon representations of molecules described herein.

FIGS. 11-15 illustrate conjugation schemes described herein.

FIG. 16 illustrates a conjugation scheme of anti-TfR1 mAb-MSTN DAR1 conjugate.

### DETAILED DESCRIPTION OF THE DISCLOSURE

Nucleic acid (e.g., RNAi) therapy is a targeted therapy with high selectivity and specificity. However, in some instances, nucleic acid therapy is also hindered by poor intracellular uptake, limited blood stability and non-specific immune stimulation. To address these issues, various modifications of the nucleic acid composition are explored, such as for example, novel linkers for better stabilizing and/or lower toxicity, optimization of binding moiety for increased target specificity and/or target delivery, and nucleic acid polymer modifications for increased stability and/or reduced off-target effect.

In some embodiments, the arrangement or order of the different components that make-up the nucleic acid composition further effects intracellular uptake, stability, toxicity, efficacy, and/or non-specific immune stimulation. For example, if the nucleic acid component includes a binding moiety, a polymer, and a polynucleic acid molecule (or polynucleotide), the order or arrangement of the binding moiety, the polymer, and/or the polynucleic acid molecule (or polynucleotide) (e.g., binding moiety-polynucleic acid molecule-polymer, binding moiety-polymer-polynucleic acid molecule, or polymer-binding moiety-polynucleic acid molecule) further effects intracellular uptake, stability, toxicity, efficacy, and/or non-specific immune stimulation.

In some embodiments, described herein include a molecule those arrangement of the nucleic acid components effects intracellular uptake, stability, toxicity, efficacy, and/or non-specific immune stimulation. In some instances, the molecule comprises a binding moiety conjugated to a polynucleic acid molecule and a polymer. In some embodiments, the molecule comprises a molecule according to Formula (I): A-X—B—Y—C; in which A is a binding moiety, B is a polynucleotide comprising at least one 5′-vinylphosphonate modified nucleotide, C is a polymer, X is a bond or first linker, and Y is a bond or second linker. In some instances, the polynucleotide comprises at least one 5′-vinylphosphonate modified nucleotide. In some instances, the molecule of Formula (I) further comprises D, an endosomolytic moiety.

In some embodiments, a molecule comprising a binding moiety conjugated to a polynucleic acid molecule and a polymer arranged as described herein enhances intracellular uptake, stability, and/or efficacy. In some instances, a molecule comprising a binding moiety conjugated to a polynucleic acid molecule and a polymer arranged as described herein reduces toxicity and/or non-specific immune stimulation. In some cases, the molecule comprises a molecule according to Formula (I): A-X—B—Y—C; in which A is a binding moiety, B is a polynucleotide comprising at least one 5′-vinylphosphonate modified nucleotide, C is a polymer, X is a bond or first linker, and Y is a bond or second linker. In some instances, the polynucleotide comprises at least one 5′-vinylphosphonate modified nucleotide. In some instances, the molecule of Formula (I) further comprises D, an endosomolytic moiety.

In some embodiments, a molecule described herein is further used to treat a disease or disorder. In some instances, a molecule for the treatment of a disease or disorder is a molecule according to Formula (I): A-X—B—Y—C; in which A is a binding moiety, B is a polynucleotide comprising at least one 5′-vinylphosphonate modified nucleotide, C is a polymer, X is a bond or first linker, and Y is a bond or second linker. In some instances, the polynucleotide comprises at least one 5′-vinylphosphonate modified nucleotide. In some instances, the molecule of Formula (I) further comprises D, an endosomolytic moiety.

In some embodiments, a molecule described herein is also used for inhibiting the expression of a target gene in a primary cell of a patient in need thereof. In such instances, a molecule for such use is a molecule according to Formula (I): A-X—B—Y—C; in which A is a binding moiety, B is a polynucleotide comprising at least one 5′-vinylphosphonate modified nucleotide, C is a polymer, X is a bond or first linker, and Y is a bond or second linker. In some instances, the polynucleotide comprises at least one 5′-vinylphosphonate modified nucleotide, at least one modified internucleotide linkage, or at least one inverted abasic moiety. In some instances, the molecule of Formula (I) further comprises D, an endosomolytic moiety.

In some embodiments, a molecule described herein is additionally used as an immuno-oncology therapy for the treatment of a disease or disorder. In some instance, the molecule is a molecule according to Formula (I): A-X—B—Y—C; in which A is a binding moiety, B is a polynucleotide comprising at least one 5′-vinylphosphonate modified nucleotide, C is a polymer, X is a bond or first linker, and Y is a bond or second linker. In some instances, the polynucleotide comprises at least one 5′-vinylphosphonate modified nucleotide, at least one modified internucleotide linkage, or at least one inverted abasic moiety. In some instances, the molecule of Formula (I) further comprises D, an endosomolytic moiety.

In additional embodiments, described herein include a kit, which comprises one or more of the molecules described herein.

### Therapeutic Molecule Platform

In some embodiments, a molecule (e.g., a therapeutic molecule) described herein comprises a binding moiety conjugated to a polynucleic acid molecule comprising at least one 5′-vinylphosphonate modified nucleotide and a polymer. In some embodiments, a molecule (e.g., a therapeutic molecule) comprises a molecule according to Formula (I):

A-X—B—Y—C Formula I

wherein,

A is a binding moiety;

B is a polynucleotide;

C is a polymer;

X is a bond or a first linker; and

Y is a bond or a second linker; and

wherein the polynucleotide comprises at least one 5′-vinylphosphonate modified nucleotide.

In some instances, the molecule of Formula (I) further comprises D, an endosomolytic moiety.

In some embodiments, at least one A and/or at least one C are conjugated to the 5′ terminus of B, the 3′ terminus of B, an internal site on B, or in any combinations thereof. In some instances, at least one A is conjugated at one terminus of B while at least one C is conjugated at the opposite terminus of B. In some instances, at least one of A is conjugated at one terminus of B while at least one of C is conjugated at an internal site on B.

In some cases, A and C are not conjugated or attached to B at the same terminus. In some cases, A is attached or conjugated to B at a first terminus of B. In some cases, C is attached or conjugated to B at a second terminus of B, and the second terminus of B is different than the first terminus. In some cases, A is attached or conjugated to B at the 5′ terminus of B, and C is attached or conjugated to B at the 3′ terminus of B. In other cases, A is attached or conjugated to B at the 3′ terminus of B, and C is attached or conjugated to B at the 5′ terminus of B.

In some embodiments, A is an antibody or binding fragment thereof. In some cases, C is a polymer. In some cases, A and C are not conjugated or attached to B at the same terminus. In some cases, A is attached or conjugated to B at a first terminus of B. In some cases, C is attached or conjugated to B at a second terminus of B, and the second terminus of B is different than the first terminus. In some cases, A is attached or conjugated to B at the 5′ terminus of B, and C is attached or conjugated to B at the 3′ terminus of B. In other cases, A is attached or conjugated to B at the 3′ terminus of B, and C is attached or conjugated to B at the 5′ terminus of B. In some cases, X which connects A to B is a bond or a non-polymeric linker. In some cases, X is a non-peptide linker (or a linker that does not comprise an amino acid residue). In some cases, Y which connects B to C is a bond or a second linker. In some instances, X connects A to the 5′ terminus of B, and Y connects C to the 3′ terminus of B. In other instances, X connects A to the 3′ terminus of B, and Y connects C to the 5′ terminus of B.

In some embodiments, X—B is conjugated or attached to the N-terminus, C-terminus, a constant region, a hinge region, or a Fc region of A. In some instances, X—B is conjugated or attached to the N-terminus of A. In some instances, X—B is conjugated or attached to the C-terminus of A. In some instances, X—B is conjugated or attached to a hinge region of A. In some instances, X—B is conjugated or attached to a constant region of A. In some instances, X—B is conjugated or attached to the Fc region of A.

In some instances, at least one B and/or at least one C, and optionally at least one D are conjugated to a first A. In some instances, the at least one B is conjugated at a terminus (e.g., a 5′ terminus or a 3′ terminus) to the first A or are conjugated via an internal site to the first A. In some cases, the at least one C is conjugated either directly to the first A or indirectly via the two or more Bs. If indirectly via the two or more Bs, the two or more Cs are conjugated either at the same terminus as the first A on B, at opposing terminus from the first A, or independently at an internal site. In some instances, at least one additional A is further conjugated to the first A, to B, or to C. In additional instances, the at least one D is optionally conjugated either directly or indirectly to the first A, to the at least one B, or to the at least one C. If directly to the first A, the at least one D is also optionally conjugated to the at least one B to form a A-D-B conjugate or is optionally conjugated to the at least one B and the at least one C to form a A-D-B—C conjugate. In some cases, the at least one additional A is different than the first A.

In some cases, two or more Bs and/or two or more Cs are conjugated to a first A. In some instances, the two or more Bs are conjugated at a terminus (e.g., a 5′ terminus or a 3′ terminus) to the first A or are conjugated via an internal site to the first A. In some instances, the two or more Cs are conjugated either directly to the first A or indirectly via the two or more Bs. If indirectly via the two or more Bs, the two or more Cs are conjugated either at the same terminus as the first A on B, at opposing terminus from the first A, or independently at an internal site. In some instances, at least one additional A is further conjugated to the first A, to two or more Bs, or to two or more Cs. In additional instances, at least one D is optionally conjugated either directly or indirectly to the first A, to the two or more Bs, or to the two or more Cs. If indirectly to the first A, the at least one D is conjugated to the first A through the two or more Bs, through the two or more Cs, through a B—C orientation to form a A-B—C-D type conjugate, or through a C—B orientation to form a A-C—B-D type conjugate. In some cases, the at least one additional A is different than the first A. In some cases, the two or more Bs are different. In other cases, the two or more Bs are the same. In some instances, the two or more Cs are different. In other instances, the two or more Cs are the same. In additional instances, the two or more Ds are different. In additional instances, the two or more Ds are the same.

In other cases, two or more Bs and/or two or more Ds, optionally two or more Cs are conjugated to a first A. In some instances, the two or more Bs are conjugated at a terminus (e.g., a 5′ terminus or a 3′ terminus) to the first A or are conjugated via an internal site to the first A. In some instances, the two or more Ds are conjugated either directly to the first A or indirectly via the two or more Bs. If indirectly via the two or more Bs, the two or more Ds are conjugated either at the same terminus as the first A on B, at opposing terminus from the first A, or independently at an internal site. In some instances, at least one additional A is further conjugated to the first A, to the two or more Bs, or to the two or more Ds. In additional instances, the two or more Cs are optionally conjugated either directly or indirectly to the first A, to the two or more Bs, or to the two or more Ds. In some cases, the at least one additional A is different than the first A. In some cases, the two or more Bs are different. In other cases, the two or more Bs are the same. In some instances, the two or more Cs are different. In other instances, the two or more Cs are the same. In additional instances, the two or more Ds are different. In additional instances, the two or more Ds are the same.

In some embodiments, a molecule (e.g., a therapeutic molecule) described herein comprises a molecule according to Formula (II):

(A-X—B—Y—C_{c})-L-D Formula II

wherein,

A is a binding moiety;

B is a polynucleotide;

C is a polymer;

X is a bond or a first linker;

Y is a bond or a second linker;

L is a bond or a third linker;

D is an endosomolytic moiety; and

c is an integer between 0 and 1; and

wherein the polynucleotide comprises at least one 5′-vinylphosphonate modified nucleotide; and D is conjugated anywhere on A, B, or C.

In some embodiments, a molecule (e.g., a therapeutic molecule) described herein comprises a molecule according to Formula (III):

Aₐ-X-B_{b}—Y—C_{c}-L-Dₙ Formula III

wherein,

A is a binding moiety;

B is a polynucleotide;

C is a polymer;

D is an endosomolytic moiety;

X is a bond or a first linker;

Y is a bond or a second linker;

L is a bond or a third linker;

a and b are independently an integer between 1-3;

c is an integer between 0 and 3; and

n is an integer between 0 and 10; and

wherein the polynucleotide comprises at least one 5′-vinylphosphonate modified nucleotide; A is conjugated anywhere on B, C, or D; B is conjugated anywhere on A, C, or D; C is conjugated anywhere on A, B, or D; and D is conjugated anywhere on A, B, or C.

In some embodiments, a molecule (e.g., a therapeutic molecule) described herein comprises a molecule according to Formula (IIIa): A-X—B-L-D-Y—C.

In some embodiments, a molecule (e.g., a therapeutic molecule) described herein comprises a molecule according to Formula (IIIb): Aₐ-X—B_{b}-L-Dₙ.

In some embodiments, a molecule (e.g., a therapeutic molecule) described herein comprises a molecule according to Formula (IV):

A-X—(B_{b}—Y—C_{c}-L-Dₙ)ₘ

wherein,

A is a binding moiety;

B is a polynucleotide;

C is a polymer;

D is an endosomolytic moiety;

X is a bond or a first linker;

Y is a bond or a second linker;

L is a bond or a third linker;

a and b are independently an integer between 1-3;

c is an integer between 0 and 3;

n is an integer between 0 and 10; and

m is an integer between 1-3; and

wherein the polynucleotide comprises at least one 5′-vinylphosphonate modified nucleotide; C is conjugated anywhere on B or D; and D is conjugated anywhere on B or C.

In some embodiments, a molecule (e.g., a therapeutic molecule) described herein comprises a molecule according to Formula (IVa): A-X—(B_{b}-L-Dₙ-Y—C_{c})ₘ.

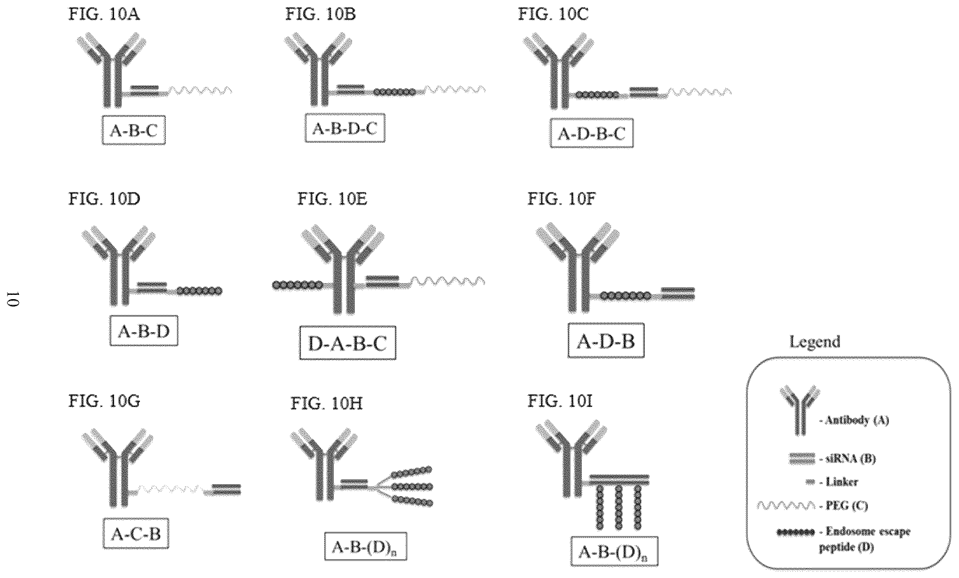

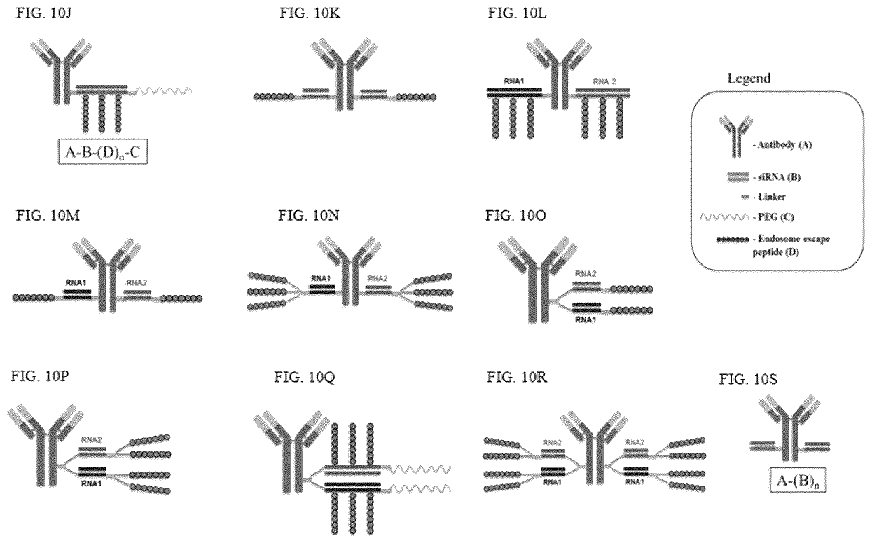

In some embodiments, a molecule (e.g., a therapeutic molecule) is a molecule as illustrated in FIG. 10A.

In some embodiments, a molecule (e.g., a therapeutic molecule) is a molecule as illustrated in FIG. 10B.

In some embodiments, a molecule (e.g., a therapeutic molecule) is a molecule as illustrated in FIG. 10C.

In some embodiments, a molecule (e.g., a therapeutic molecule) is a molecule as illustrated in FIG. 10D.

In some embodiments, a molecule (e.g., a therapeutic molecule) is a molecule as illustrated in FIG. 10E.

In some embodiments, a molecule (e.g., a therapeutic molecule) is a molecule as illustrated illustrated in FIG. 10F.

In some embodiments, a molecule (e.g., a therapeutic molecule) is a molecule as illustrated in FIG. 10G.

In some embodiments, a molecule (e.g., a therapeutic molecule) is a molecule as illustrated in FIG. 10H.

In some embodiments, a molecule (e.g., a therapeutic molecule) is a molecule as illustrated in FIG. 10I.

In some embodiments, a molecule (e.g., a therapeutic molecule) is a molecule as illustrated in FIG. 10J.

In some embodiments, a molecule (e.g., a therapeutic molecule) is a molecule as illustrated in FIG. 10K.

In some embodiments, a molecule (e.g., a therapeutic molecule) is a molecule as illustrated in FIG. 10L.

In some embodiments, a molecule (e.g., a therapeutic molecule) is a molecule as illustrated in FIG. 10M.

In some embodiments, a molecule (e.g., a therapeutic molecule) is a molecule as illustrated in FIG. 10N.

In some embodiments, a molecule (e.g., a therapeutic molecule) is a molecule as illustrated in FIG. 10O.

In some embodiments, a molecule (e.g., a therapeutic molecule) is a molecule as illustrated in FIG. 10P.

In some embodiments, a molecule (e.g., a therapeutic molecule) is a molecule as illustrated in FIG. 10Q.

In some embodiments, a molecule (e.g., a therapeutic molecule) is a molecule as illustrated in FIG. 10R.

In some embodiments, a molecule (e.g., a therapeutic molecule) is a molecule as illustrated in FIG. 10S.

The antibody as illustrated above is for representation purposes only and encompasses a humanized antibody or binding fragment thereof, chimeric antibody or binding fragment thereof, monoclonal antibody or binding fragment thereof, monovalent Fab′, divalent Fab2, single-chain variable fragment (scFv), diabody, minibody, nanobody, single-domain antibody (sdAb), or camelid antibody or binding fragment thereof.

### Polynucleic Acid Molecule Targets

In some embodiments, the polynucleic acid molecule B is a polynucleic acid molecule (or polynucleotide) that hybridizes to a target region on an oncogene. In some instances, oncogenes are further classified into several categories: growth factors or mitogens, receptor tyrosine kinases, cytoplasmic tyrosine kinases, cytoplasmic serine/threonine kinases, regulatory GTPases, and transcription factors. Exemplary growth factors include c-Sis. Exemplary receptor tyrosine kinases include epidermal growth factor receptor (EGFR), platelet-derived growth factor receptor (PDGFR), vascular endothelial growth factor receptor (VEGFR), and HER2/neu. Exemplary cytoplasmic tyrosine kinases include Src-family tyrosine kinases, Syk-ZAP-70 family of tyrosine kinases, BTK family of tyrosine kinases, and Abl gene in CML. Exemplary cytoplasmic serine/threonine kinases include Raf kinase and cyclin-dependent kinases. Exemplary regulatory GTPases include Ras family of proteins such as KRAS. Exemplary transcription factors include MYC gene. In some instances, an oncogene described herein comprises an oncogene selected from growth factors or mitogens, receptor tyrosine kinases, cytoplasmic tyrosine kinases, cytoplasmic serine/threonine kinases, regulatory GTPases, or transcription factors. In some embodiments, the polynucleic acid molecule is a polynucleic acid molecule that hybridizes to a target region of an oncogene selected from growth factors or mitogens, receptor tyrosine kinases, cytoplasmic tyrosine kinases, cytoplasmic serine/threonine kinases, regulatory GTPases, or transcription factors.

In some embodiments, an oncogene described herein comprises Abl, AKT-2, ALK, AML1 (or RUNX1), AR, AXL, BCL-2, 3, 6, BRAF, c-MYC, EGFR, ErbB-2 (Her2, Neu), Fms, FOS, GLI1, HPRTI, IL-3, INTS2, JUN, KIT, KS3, K-sam, LBC (AKAP13), LCK, LMOI, LMO2, LYL1, MAS1, MDM2, MET, MLL (KMT2A), MOS, MYB, MYH11/CBFB, NOTCH1 (TAN1), NTRKI (TRK), OST (SLC51B), PAX5, PIM1, PRAD-1, RAF, RAR/PML, HRAS, KRAS, NRAS, REL/NRG, RET, ROS, SKI, SRC, TIAM1, or TSC2. In some embodiments, the polynucleic acid molecule is a polynucleic acid molecule that hybridizes to a target region of Abl, AKT-2, ALK, AML1 (or RUNX1), AR, AXL, BCL-2, 3, 6, BRAF, c-MYC, EGFR, ErbB-2 (Her2, Neu), Fms, FOS, GLI1, HPRT1, IL-3, INTS2, JUN, KIT, KS3, K-sam, LBC (AKAP13), LCK, LMOI, LMO2, LYL1, MAS1, MDM2, MET, MLL (KMT2A), MOS, MYB, MYH11/CBFB, NOTCH1 (TAN1), NTRK1 (TRK), OST (SLC51B), PAX5, PIM1, PRAD-1, RAF, AR/PML, HRAS, KRAS, NRAS, REL/NRG, RET, ROS, SKI, SRC, TIAM1, or TSC2.

In some embodiments, an oncogene described herein comprises KRAS, EGFR, AR, HPRT1, CNNTB1 (β-catenin), or β-catenin associated genes. In some embodiments, the polynucleic acid molecule B is a polynucleic acid molecule that hybridizes to a target region of KRAS, EGFR, AR, HPRT1, CNNTB1 (β-catenin), or β-catenin associated genes. In some embodiments, the polynucleic acid molecule B is a polynucleic acid molecule that hybridizes to a target region of KRAS. In some embodiments, the polynucleic acid molecule B is a polynucleic acid molecule that hybridizes to a target region of EGFR. In some embodiments, the polynucleic acid molecule B is a polynucleic acid molecule that hybridizes to a target region of AR. In some embodiments, the polynucleic acid molecule B is a polynucleic acid molecule that hybridizes to a target region of CNNTB1 (β-catenin). In some embodiments, the polynucleic acid molecule B is a polynucleic acid molecule that hybridizes to a target region of CNNTB1 (β-catenin) associated genes. In some instances, the β-catenin associated genes comprise PIK3CA, PIK3CB, and Myc. In some instances, the polynucleic acid molecule B is a polynucleic acid molecule that hybridizes to a target region of HPRT1.

Polynucleic Acid Molecules that Target Kirsten Rat Sarcoma Viral Oncogene Homolog (KRAS)

Kirsten Rat Sarcoma Viral Oncogene Homolog (also known as GTPase KRas, V-Ki-ras2 Kirsten rat sarcoma viral oncogene homolog, or KRAS) is involved in regulating cell division. The K-Ras protein is a GTPase belonging to the Ras superfamily. In some instances, K-Ras modulates cell cycle progression, as well as induces growth arrest, apoptosis, and replicative senescence under different environmental triggers (e.g., cellular stress, ultraviolet, heat shock, or ionizing irradiation). In some cases, wild type KRAS gene has been shown to be frequently lost during tumor progression in different types of cancer, while mutations of KRAS gene have been linked to cancer development. In some instances, KRAS amplification has also been implicated in cancer development (see, for example, Valtorta et al. “KRAS gene amplification in colorectal cancer and impact on response to EGFR-targeted therapy,” Int. J. Cancer 133: 1259-1266 (2013)). In such cases, the cancer pertains to a refractory cancer in which the patient has acquired resistance to a particular inhibitor or class of inhibitors.

In some embodiments, the KRAS gene is wild type or comprises a mutation. In some instances, KRAS mRNA is wild type or comprises a mutation. In some instances, the polynucleic acid molecule is a polynucleic acid molecule that hybridizes to a target region of wild type KRAS DNA or RNA. In some instances, the polynucleic acid molecule is a polynucleic acid molecule that hybridizes to a target region of KRAS DNA or RNA comprising a mutation (e.g., a substitution, a deletion, or an addition).

In some embodiments, KRAS DNA or RNA comprises one or more mutations. In some embodiments, KRAS DNA or RNA comprises one or more mutations at codons 12 or 13 in exon 1. In some instances, KRAS DNA or RNA comprises one or more mutations at codons 61, 63, 117, 119, or 146. In some instances, KRAS DNA or RNA comprises one or more mutations at positions corresponding to amino acid residues 12, 13, 18, 19, 20, 22, 24, 26, 36, 59, 61, 63, 64, 68, 110, 116, 117, 119, 146, 147, 158, 164, 176, or a combination thereof of the KRAS polypeptide. In some embodiments, KRAS DNA or RNA comprises one or more mutations at positions corresponding to amino acid residues selected from G12V, G12D, G12C, G12A, G12S, G12F, G13C, G13D, G13V, A18D, L19F, T20R, Q22K, I24N, N26K, I36L, I36M, A59G, A59E, Q61K, Q61H, Q61L, Q61R, E63K, Y64D, Y64N, R68S, P110S, K117N, C118S, A146T, A146P, A146V, K147N, T158A, R164Q, K176Q, or a combination thereof of the KRAS polypeptide.

In some embodiments, the polynucleic acid molecule hybridizes to a target region of KRAS DNA or RNA comprising one or more mutations. In some embodiments, the polynucleic acid molecule hybridizes to a target region of KRAS DNA or RNA comprising one or more mutations at codons 12 or 13 in exon 1. In some embodiments, the polynucleic acid molecule hybridizes to a target region of KRAS DNA or RNA comprising one or more mutations at codons 61, 63, 117, 119, or 146. In some embodiments, the polynucleic acid molecule hybridizes to a target region of KRAS DNA or RNA comprising one or more mutations at positions corresponding to amino acid residues 12, 13, 18, 19, 20, 22, 24, 26, 36, 59, 61, 63, 64, 68, 110, 116, 117, 119, 146, 147, 158, 164, 176, or a combination thereof of the KRAS polypeptide. In some embodiments, the polynucleic acid molecule hybridizes to a target region ofKRAS DNA or RNA comprising one or more mutations corresponding to amino acid residues selected from G12V, G12D, G12C, G12A, G12S, G12F, G13C, G13D, G13V, A18D, L19F, T20R, Q22K, I24N, N26K, I36L, I36M, A59G, A59E, Q61K, Q61H, Q61L, Q61R, E63K, Y64D, Y64N, R68S, P110S, K117N, C118S, A146T, A146P, A146V, K147N, T158A, R164Q, K176Q, or a combination thereof of the KRAS polypeptide.

### Polynucleic Acid Molecules That Target Epidermal Growth Factor Receptor (EGFR)

Epidermal growth factor receptor (EGFR, ErbB-1, or HER1) is a transmembrane tyrosine kinase receptor and a member of the ErbB family of receptors, which also include HER2/c-neu (ErbB-2), Her3 (ErbB-3) and Her4 (ErbB-4). In some instances, EGFR mutations drive the downstream activation of RAS/RAF/MAPK, PI3K/AKT, and/or JAK/STAT pathways, leading to mitosis, cell proliferation, and suppression of apoptosis. In addition, amplification of wild-type EGFR gene has been implicated in the development of cancers such as glioblastomas and non-small cell lung cancer (Talasila, et al., “EGFR Wild-type Amplification and Activation Promote Invasion and Development of Glioblastoma Independent of Angiogenesis,” Acta Neuropathol. 125(5): 683-698 (2013); Bell et al., “Epidermal Growth Factor Receptor Mutations and Gene Amplification in Non-Small-Cell Lung Cancer: Molecular Analysis of the IDEAL/INTACT Gefitinib Trials,” J. Clinical Oncology 23(31): 8081-8092 (2005)).

In some embodiments, EGFR DNA or RNA is wild type EGFR or EGFR comprising a mutation. In some instances, EGFR is wild type EGFR. In some instances, EGFR DNA or RNA comprises a mutation. In some instances, the polynucleic acid molecule hybridizes to a target region of wild type EGFR DNA or RNA. In some instances, the polynucleic acid molecule hybridizes to a target region of EGFR DNA or RNA comprising a mutation (e.g., a substitution, a deletion, or an addition).

In some instances, EGFR DNA or RNA comprises one or more mutations. In some embodiments, EGFR DNA or RNA comprises one or more mutations within one or more exons. In some instances, the one or more exons comprise exon 18, exon 19, exon 20, exon 21 or exon 22. In some instances, EGFR DNA or RNA comprises one or more mutations in exon 18, exon 19, exon 20, exon 21, exon 22 or a combination thereof.

In some instances, EGFR DNA or RNA comprises one or more mutations at positions corresponding to amino acid residues 34, 38, 45, 62, 63, 77, 78, 108, 114, 120, 140, 148, 149, 160, 177, 178, 189, 191, 198, 220, 222, 223, 229, 237, 240, 244, 252, 254, 255, 256, 263, 270, 273, 276, 282, 288, 289, 301, 303, 304, 309, 314, 326, 331, 354, 363, 373, 337, 380, 384, 393, 427, 428, 437, 441, 447, 465, 475, 515, 526, 527, 531, 536, 541, 546, 571, 588, 589, 596, 596, 598, 602, 614, 620, 628, 636, 641, 645, 651, 671, 689, 694, 700, 709, 712, 714, 715, 716, 719, 720, 721, 731, 733, 739-744, 742, 746-750, 746-752, 746, 747, 747-749, 747-751, 747-753, 751, 752, 754, 752-759, 750, 761-762, 761, 763, 765, 767-768, 767-769, 768, 769, 769-770, 770-771, 772, 773-774, 773, 774, 774-775, 776, 779, 783, 784, 786, 790, 792, 794, 798, 803, 805, 807, 810, 826, 827, 831, 832, 833, 835, 837, 838, 839, 842, 843, 847, 850, 851, 853, 854, 856, 858, 861, 863, 894, 917, 967, 1006, 1019, 1042, 1100, 1129, 1141, 1153, 1164, 1167, or a combination thereof of the EGFR polypeptide. In some embodiments, EGFR DNA or RNA comprises one or more mutations at positions corresponding to amino acid residues 747, 761, 790, 854, 858, or a combination thereof of the EGFR polypeptide. In some embodiments, EGFR DNA or RNA comprises one or more mutations at positions corresponding to amino acid residues 761, 790, 858, or a combination thereof of the EGFR polypeptide. In some embodiments, EGFR DNA or RNA comprises a mutation at a position corresponding to amino acid residue 747 of the EGFR polypeptide. In some embodiments, EGFR DNA or RNA comprises a mutation at a position corresponding to amino acid residue 761 of the EGFR polypeptide. In some embodiments, EGFR DNA or RNA comprises a mutation at a position corresponding to amino acid residue 790 of the EGFR polypeptide. In some embodiments, EGFR DNA or RNA comprises a mutation at a position corresponding to amino acid residue 854 of the EGFR polypeptide. In some embodiments, EGFR DNA or RNA comprises a mutation at a position corresponding to amino acid residue 858 of the EGFR polypeptide.

In some embodiments, EGFR DNA or RNA comprises one or more mutations selected from T34M, L38V, E45Q, L62R, G63R, G63K, S77F, F78L, R108K, R108G, E114K, A120P, L140V, V148M, R149W, E160K, S177P, M178I, K189T, D191N, S198R, S220P, R222L, R222C, S223Y, S229C, A237Y, C240Y, R244G, R252C, R252P, F254I, R255 (nonsense mutation), D256Y, T263P, Y270C, T273A, Q276 (nonsense), E282K, G288 (frame shift), A289D, A289V, A289T, A289N, A289D, V301 (deletion), D303H, H304Y, R309Q, D314N, C326R, G331R, T354M, T363I, P373Q, R337S, S380 (frame shift), T384S, D393Y, R427L, G428S, S437Y, V441I, S447Y, G465R, I475V, C515S, C526S, R527L, R531 (nonsense), V536M, L541I, P546Q, C571S, G588S, P589L, P596L, P596S, P596R, P596L, G598V, G598A, E602G, G614D, C620Y, C620W, C628Y, C628F, C636Y, T638M, P641H, S645C, V651M, R671C, V689M, P694S, N700D, E709A, E709K, E709Q, E709K, F712L, K714N, I715S, K716R, G719A, G719C, G719D, G719S, S720C, S720F, G721V, W731Stop, P733L, K739-I744 (insertion), V7421, V742A, E746-A750 (deletion), E746K, L747S, L747-E749 (deletion), L747-T751 (deletion), L747-P753 (deletion), G746-S752 (deletion), T751I, S752Y, K754 (deletion), S752-1759 (deletion), A750P, D761-E762 (e.g., residues EAFQ insertion (SEQ ID NO: 1276)), D761N, D761Y, A763V, V765A, A767-S768 (e.g., residues TLA insertion), A767-V769 (e.g., residues ASV insertion), S7681, S768T, V769L, V769M, V769-D770 (e.g., residue Y insertion), 770-771 (e.g., residues GL insertion), 770-771 (e.g., residue G insertion), 770-771 (e.g., residues CV insertion), 770-771 (e.g., residues SVD insertion), P772R, 773-774 (e.g., residues NPH insertion), H773R, H773L, V774M, 774-775 (e.g., residues HV insertion), R776H, R776C, G779F, T783A, T784F, T854A, V786L, T790M, L792P, P794H, L798F, R803W, H805R, D807H, G810S, N826S, Y827 (nonsense), R831H, R832C, R832H, L833F, L833V, H835L, D837V, L838M, L838P, A839V, N842H, V843L, T847K, T8471, H850N, V851A, 1853T, F856L, L858R, L858M, L861Q, L861R, G863D, Q894L, G917A, E967A, D1006Y, P1019L, S1042N, R1100S, H1129Y, T1141S, S1153I, Q1164R, L1167M, or a combination thereof of the EGFR polypeptide.

In some instances, the polynucleic acid molecule hybridizes to a target region of EGFR DNA or RNA comprising one or more mutations. In some embodiments, the polynucleic acid molecule hybridizes to a target region of EGFR DNA or RNA comprising one or more mutations in exon 18, exon 19, exon 20, exon 21, exon 22 or a combination thereof.

In some embodiments, the polynucleic acid molecule hybridizes to a target region of EGFR DNA or RNA comprising one or more mutations at positions corresponding to amino acid residues 34, 38, 45, 62, 63, 77, 78, 108, 114, 120, 140, 148, 149, 160, 177, 178, 189, 191, 198, 220, 222, 223, 229, 237, 240, 244, 252, 254, 255, 256, 263, 270, 273, 276, 282, 288, 289, 301, 303, 304, 309, 314, 326, 331, 354, 363, 373, 337, 380, 384, 393, 427, 428, 437, 441, 447, 465, 475, 515, 526, 527, 531, 536, 541, 546, 571, 588, 589, 596, 596, 598, 602, 614, 620, 628, 636, 641, 645, 651, 671, 689, 694, 700, 709, 712, 714, 715, 716, 719, 720, 721, 731, 733, 739-744, 742, 746-750, 746-752, 746, 747, 747-749, 747-751, 747-753, 751, 752, 754, 752-759, 750, 761-762, 761, 763, 765, 767-768, 767-769, 768, 769, 769-770, 770-771, 772, 773-774, 773, 774, 774-775, 776, 779, 783, 784, 786, 790, 792, 794, 798, 803, 805, 807, 810, 826, 827, 831, 832, 833, 835, 837, 838, 839, 842, 843, 847, 850, 851, 853, 854, 856, 858, 861, 863, 894, 917, 967, 1006, 1019, 1042, 1100, 1129, 1141, 1153, 1164, 1167, or a combination thereof of the EGFR polypeptide. In some embodiments, the polynucleic acid molecule hybridizes to a target region of EGFR DNA or RNA comprising one or more mutations at positions corresponding to amino acid residues 747, 761, 790, 854, 858, or a combination thereof of the EGFR polypeptide. In some embodiments, the polynucleic acid molecule hybridizes to a target region of EGFR DNA or RNA comprising one or more mutations at positions corresponding to amino acid residues 761, 790, 858, or a combination thereof of the EGFR polypeptide. In some embodiments, the polynucleic acid molecule hybridizes to a target region of EGFR DNA or RNA comprising a mutation at a position corresponding to amino acid residue 747 of the EGFR polypeptide. In some embodiments, the polynucleic acid molecule hybridizes to a target region of EGFR DNA or RNA comprising a mutation at a position corresponding to amino acid residue 761 of the EGFR polypeptide. In some embodiments, the polynucleic acid molecule hybridizes to a target region of EGFR DNA or RNA comprising a mutation at a position corresponding to amino acid residue 790 of the EGFR polypeptide. In some embodiments, the polynucleic acid molecule hybridizes to a target region of EGFR DNA or RNA comprising a mutation at a position corresponding to amino acid residue 854 of the EGFR polypeptide. In some embodiments, the polynucleic acid molecule hybridizes to a target region of EGFR DNA or RNA comprising a mutation at a position corresponding to amino acid residue 858 of the EGFR polypeptide.

In some embodiments, the polynucleic acid molecule hybridizes to a target region of EGFR DNA or RNA comprising one or more mutations selected from T34M, L38V, E45Q, L62R, G63R, G63K, S77F, F78L, R108K, R108G, E114K, A120P, L140V, V148M, R149W, E160K, S177P, M178I, K189T, D191N, S198R, S220P, R222L, R222C, S223Y, S229C, A237Y, C240Y, R244G, R252C, R252P, F2541, R255 (nonsense mutation), D256Y, T263P, Y270C, T273A, Q276 (nonsense), E282K, G288 (frame shift), A289D, A289V, A289T, A289N, A289D, V301 (deletion), D303H, H304Y, R309Q, D314N, C326R, G331R, T354M, T363I, P373Q, R337S, S380 (frame shift), T384S, D393Y, R427L, G428S, S437Y, V4411, S447Y, G465R, 1475V, C515S, C526S, R527L, R531 (nonsense), V536M, L541I, P546Q, C571S, G588S, P589L, P596L, P596S, P596R, P596L, G598V, G598A, E602G, G614D, C620Y, C620W, C628Y, C628F, C636Y, T638M, P641H, S645C, V651M, R671C, V689M, P694S, N700D, E709A, E709K, E709Q, E709K, F712L, K714N, I715S, K716R, G719A, G719C, G719D, G719S, S720C, S720F, G721V, W731Stop, P733L, K739-I744 (insertion), V742I, V742A, E746-A750 (deletion), E746K, L747S, L747-E749 (deletion), L747-T751 (deletion), L747-P753 (deletion), G746-S752 (deletion), T751I, S752Y, K754 (deletion), S752-1759 (deletion), A750P, D761-E762 (e.g., residues EAFQ insertion (SEQ ID NO: 1276)), D761N, D761Y, A763V, V765A, A767-S768 (e.g., residues TLA insertion), A767-V769 (e.g., residues ASV insertion), S7681, S768T, V769L, V769M, V769-D770 (e.g., residue Y insertion), 770-771 (e.g., residues GL insertion), 770-771 (e.g., residue G insertion), 770-771 (e.g., residues CV insertion), 770-771 (e.g., residues SVD insertion), P772R, 773-774 (e.g., residues NPH insertion), H773R, H773L, V774M, 774-775 (e.g., residues HV insertion), R776H, R776C, G779F, T783A, T784F, T854A, V786L, T790M, L792P, P794H, L798F, R803W, H805R, D807H, G810S, N826S, Y827 (nonsense), R831H, R832C, R832H, L833F, L833V, H835L, D837V, L838M, L838P, A839V, N842H, V843L, T847K, T8471, H850N, V851A, 1853T, F856L, L858R, L858M, L861Q, L861R, G863D, Q894L, G917A, E967A, D1006Y, P1019L, S1042N, R1100S, Hi129Y, T1141S, S1153I, Q1164R, L 1167M, or a combination thereof of the EGFR polypeptide. In some embodiments, the polynucleic acid molecule hybridizes to a target region of EGFR DNA or RNA comprising one or more mutations selected from L747S, D761Y, T790M, T854A, L858R, or a combination thereof of the EGFR polypeptide. In some embodiments, the polynucleic acid molecule hybridizes to a target region of EGFR DNA or RNA comprising one or more mutations selected from D761Y, T790M, L858R, or a combination thereof of the EGFR polypeptide. In some embodiments, the polynucleic acid molecule hybridizes to a target region of EGFR DNA or RNA comprising mutation L747S of the EGFR polypeptide. In some embodiments, the polynucleic acid molecule hybridizes to a target region of EGFR DNA or RNA comprising mutation D761Y of the EGFR polypeptide. In some embodiments, the polynucleic acid molecule hybridizes to a target region of EGFR DNA or RNA comprising mutation T790M of the EGFR polypeptide. In some embodiments, the polynucleic acid molecule hybridizes to a target region of EGFR DNA or RNA comprising mutation T854A of the EGFR polypeptide. In some embodiments, the polynucleic acid molecule hybridizes to a target region of EGFR DNA or RNA comprising mutation L858R of the EGFR polypeptide.

### Polynucleic Acid Molecules That Target Androgen Receptor (AR)

Androgen receptor (AR) (also known as NR3C4, nuclear receptor subfamily 3, group C, gene 4) belongs to the steroid hormone group of nuclear receptor superfamily along with related members: estrogen receptor (ER), glucocorticoid receptor (GR), progesterone receptor (PR), and mineralocorticoid receptor (MR). Androgens, or steroid hormones, modulate protein synthesis and tissue remodeling through the androgen receptor. The AR protein is a ligand-inducible zinc finger transcription factor that regulates target gene expression. The presence of mutations in the AR gene has been observed in several types of cancers (e.g., prostate cancer, breast cancer, bladder cancer, or esophageal cancer), and in some instances, has been linked to metastatic progression.

In some embodiments, AR DNA or RNA is wild type or comprises one or more mutations and/or splice variants. In some instances, AR DNA or RNA comprises one or more mutations. In some instances, AR DNA or RNA comprises one or more splice variants selected from AR splice variants including but not limited to AR1/2/2b, ARV2, ARV3, ARV4, AR1/2/3/2b, ARV5, ARV6, ARV7, ARV9, ARV10, ARV11, ARV12, ARV13, ARV14, ARV15, ARV16, and ARV(v567es). In some instances, the polynucleic acid molecule hybridizes to a target region of AR DNA or RNA comprising a mutation (e.g., a substitution, a deletion, or an addition) or a splice variant.

In some embodiments, AR DNA or RNA comprises one or more mutations. In some embodiments, AR DNA or RNA comprises one or more mutations within one or more exons. In some instances, the one or more exons comprise exon 1, exon 2, exon 3, exon 4, exon 5, exon 6, exon 7, or exon 8. In some embodiments, AR DNA or RNA comprises one or more mutations within exon 1, exon 2, exon 3, exon 4, exon 5, exon 6, exon 7, exon 8 or a combination thereof. In some instances, AR DNA or RNA comprises one or more mutations at positions corresponding to amino acid residues 2, 14, 16, 29, 45, 54, 57, 64, 106, 112, 176, 180, 184, 194, 198, 204, 214, 221, 222, 233, 243, 252, 255, 266, 269, 287, 288, 334, 335, 340, 363, 368, 369, 390, 403, 443, 491, 505, 513, 524, 524, 528, 533, 547, 548, 564, 567, 568, 574, 547, 559, 568, 571, 573, 575, 576, 577, 578, 579, 580, 581, 582, 585, 586, 587, 596, 597, 599, 601, 604, 607, 608, 609, 610, 611, 615, 616, 617, 619, 622, 629, 630, 638, 645, 647, 653, 662, 664, 670, 671, 672, 674, 677, 681, 682, 683, 684, 687, 688, 689, 690, 695, 700, 701, 702, 703, 705, 706, 707, 708, 710, 711, 712, 715, 717, 720, 721, 722, 723, 724, 725, 726, 727, 728, 730, 732, 733, 737, 739, 741, 742, 743, 744, 745, 746, 748, 749, 750, 751, 752, 754, 755, 756, 757, 758, 759, 762, 763, 764, 765, 766, 767, 768, 771, 772, 774, 777, 779, 786, 795, 780, 782, 784, 787, 788, 790, 791, 793, 794, 798, 802, 803, 804, 806, 807, 812, 813, 814, 819, 820, 821, 824, 827, 828, 830, 831, 834, 840, 841, 842, 846, 854, 855, 856, 863, 864, 866, 869, 870, 871, 874, 875, 877, 879, 880, 881, 886, 888, 889, 891, 892, 895, 896, 897, 898, 902, 903, 904, 907, 909, 910, 911, 913, 916, 919, or a combination thereof of the AR polypeptide. In some embodiments, AR DNA or RNA comprises one or more mutations at positions corresponding to amino acid residues selected from E2K, P14Q, K16N, V29M, S45T, L54S, L57Q, Q64R, Y106C, Q112H, S176S, K180R, L184P, Q194R, E198G, G204S, G214R, K221N, N222D, D233K, S243L, A252V, L255P, M266T, P269S, A287D, E288K, S334P, S335T, P340L, Y363N, L368V, A369P, P390R, P390S, P390L, A403V, Q443R, G491S, G505D, P513S, G524D, G524S, D528G, P533S, L547F, P548S, D564Y, S567F, G568W, L574P, L547F, C559Y, G568W, G568V, Y571C, Y571H, A573D, T575A, C576R, C576F, G577R, S578T, C579Y, C579F, K580R, V581F, F582Y, F582S, R585K, A586V, A587S, A596T, A596S, S597G, S597I, N599Y, C601F, D604Y, R607Q, R608K, K609N, D610T, C611Y, R615H, R615P, R615G, R616C, L616R, L616P, R617P, C619Y, A622V, R629W, R629Q, K630T, L638M, A645D, S647N, E653K, S662 (nonsense), I664N, Q670L, Q670R, P671H, I672T, L674P, L677P, E681L, P682T, G683A, V684I, V684A, A687V, G688Q, H689P, D690V, D695N, D695V, D695H, L700M, L701P, L701I, H701H, S702A, S703G, N705S, N705Y, E706 (nonsense), L707R, G708A, R710T, Q711E, L712F, V715M, K717Q, K720E, A721T, L722F, P723S, G724S, G724D, G724N, F725L, R726L, N727K, L728S, L728I, V730M, D732N, D732Y, D732E, Q733H, I737T, Y739D, W741R, M742V, M742I, G743R, G743V, L744F, M745T, V746M, A748D, A748V, A748T, M749V, M749I, G750S, G750D, W751R, R752Q, F754V, F754L, T755A, N756S, N756D, V757A, N758T, S759F, S759P, L762F, Y763H, Y763C, F764L, A765T, A765V, P766A, P766S, D767E, L768P, L768M, N771H, E772G, E772A, R774H, R774C, K777T, R779W, R786Q, G795V, M780I, S782N, C784Y, M787V, R788S, L790F, S791P, E793D, F794S, Q798E, Q802R, G803L, F804L, C806Y, M807V, M807R, M807I, L812P, F813V, S814N, N819Q, G820A, L821V, Q824L, Q824R, F827L, F827V, D828H, L830V, L830P, R831Q, R831L, Y834C, R840C, R840H, I841S, I842T, R846G, R854K, R855C, R855H, F856L, L863R, D864N, D864E, D864G, V866L, V866M, V866E, I869M, A870G, A870V, R871G, H874Y, H874R, Q875K, T877S, T877A, D879T, D879G, L880Q, L881V, M886V, S888L, V889M, F891L, P892L, M895T, A896T, E897D, I898T, Q902R, V903M, P904S, P904H, L907F, G909R, G909E, K910R, V911L, P913S, F916L, Q919R, or a combination thereof of the AR polypeptide.

In some embodiments, the polynucleic acid molecule hybridizes to a target region ofAR DNA or RNA comprising one or more mutations. In some embodiments the polynucleic acid hybridizes to one or more AR splice variants. In some embodiments the polynucleic acid hybridizes to AR DNA or RNA comprising one or more AR splice variants including but not limited to AR1/2/2b, ARV2, ARV3, ARV4, AR1/2/3/2b, ARV5, ARV6, ARV7, ARV9, ARV10, ARV11, ARV12, ARV13, ARV14, ARV15, ARV16, and ARV(v567es). In some embodiments, the polynucleic acid molecule hybridizes to a target region of AR DNA or RNA comprising one or more mutations within exon 1, exon 2, exon 3, exon 4, exon 5, exon 6, exon 7, exon 8 or a combination thereof. In some embodiments, the polynucleic acid molecule hybridizes to a target region of AR DNA or RNA comprising one or more mutations at positions corresponding to amino acid residues 2, 14, 16, 29, 45, 54, 57, 64, 106, 112, 176, 180, 184, 194, 198, 204, 214, 221, 222, 233, 243, 252, 255, 266, 269, 287, 288, 334, 335, 340, 363, 368, 369, 390, 403, 443, 491, 505, 513, 524, 524, 528, 533, 547, 548, 564, 567, 568, 574, 547, 559, 568, 571, 573, 575, 576, 577, 578, 579, 580, 581, 582, 585, 586, 587, 596, 597, 599, 601, 604, 607, 608, 609, 610, 611, 615, 616, 617, 619, 622, 629, 630, 638, 645, 647, 653, 662, 664, 670, 671, 672, 674, 677, 681, 682, 683, 684, 687, 688, 689, 690, 695, 700, 701, 702, 703, 705, 706, 707, 708, 710, 711, 712, 715, 717, 720, 721, 722, 723, 724, 725, 726, 727, 728, 730, 732, 733, 737, 739, 741, 742, 743, 744, 745, 746, 748, 749, 750, 751, 752, 754, 755, 756, 757, 758, 759, 762, 763, 764, 765, 766, 767, 768, 771, 772, 774, 777, 779, 786, 795, 780, 782, 784, 787, 788, 790, 791, 793, 794, 798, 802, 803, 804, 806, 807, 812, 813, 814, 819, 820, 821, 824, 827, 828, 830, 831, 834, 840, 841, 842, 846, 854, 855, 856, 863, 864, 866, 869, 870, 871, 874, 875, 877, 879, 880, 881, 886, 888, 889, 891, 892, 895, 896, 897, 898, 902, 903, 904, 907, 909, 910, 911, 913, 916, 919, ora combination thereof of the AR polypeptide. In some embodiments, the polynucleic acid molecule hybridizes to a target region of AR DNA or RNA comprising one or more mutations selected from E2K, P14Q, K16N, V29M, S45T, L54S, L57Q, Q64R, Y106C, Q112H, S176S, K180R, L184P, Q194R, E198G, G204S, G214R, K221N, N222D, D233K, S243L, A252V, L255P, M266T, P269S, A287D, E288K, S334P, S335T, P340L, Y363N, L368V, A369P, P390R, P390S, P390L, A403V, Q443R, G491S, G505D, P513S, G524D, G524S, D528G, P533S, L547F, P548S, D564Y, S567F, G568W, L574P, L547F, C559Y, G568W, G568V, Y571C, Y571H, A573D, T575A, C576R, C576F, G577R, S578T, C579Y, C579F, K580R, V581F, F582Y, F582S, R585K, A586V, A587S, A596T, A596S, S597G, S597I, N599Y, C601F, D604Y, R607Q, R608K, K609N, D610T, C611Y, R615H, R615P, R615G, R616C, L616R, L616P, R617P, C619Y, A622V, R629W, R629Q, K630T, L638M, A645D, S647N, E653K, S662 (nonsense), I664N, Q670L, Q670R, P671H, I672T, L674P, L677P, E681L, P682T, G683A, V684I, V684A, A687V, G688Q, H689P, D690V, D695N, D695V, D695H, L700M, L701P, L701I, H701H, S702A, S703G, N705S, N705Y, E706 (nonsense), L707R, G708A, R710T, Q711E, L712F, V715M, K717Q, K720E, A721T, L722F, P723S, G724S, G724D, G724N, F725L, R726L, N727K, L728S, L728I, V730M, D732N, D732Y, D732E, Q733H, I737T, Y739D, W741R, M742V, M742I, G743R, G743V, L744F, M745T, V746M, A748D, A748V, A748T, M749V, M749I, G750S, G750D, W751R, R752Q, F754V, F754L, T755A, N756S, N756D, V757A, N758T, S759F, S759P, L762F, Y763H, Y763C, F764L, A765T, A765V, P766A, P766S, D767E, L768P, L768M, N771H, E772G, E772A, R774H, R774C, K777T, R779W, R786Q, G795V, M780I, S782N, C784Y, M787V, R788S, L790F, S791P, E793D, F794S, Q798E, Q802R, G803L, F804L, C806Y, M807V, M807R, M807I, L812P, F813V, S814N, N819Q, G820A, L821V, Q824L, Q824R, F827L, F827V, D828H, L830V, L830P, R831Q, R831L, Y834C, R840C, R840H, I841S, I842T, R846G, R854K, R855C, R855H, F856L, L863R, D864N, D864E, D864G, V866L, V866M, V866E, I869M, A870G, A870V, R871G, H874Y, H874R, Q875K, T877S, T877A, D879T, D879G, L880Q, L881V, M886V, S888L, V889M, F891L, P892L, M895T, A896T, E897D, I898T, Q902R, V903M, P904S, P904H, L907F, G909R, G909E, K910R, V911L, P913S, F916L, Q919R, or a combination thereof of the AR polypeptide.

Polynucleic Acid Molecules that Target B-Catenin and B-Catenin-Associated Genes

Catenin beta-1 (also known as CTNNB1, β-catenin, or beta-catenin) is a member of the catenin protein family. In humans, it is encoded by the CTNNBI gene and is known for its dual functions—cell-cell adhesion and gene transcription. Beta-catenin is an integral structural component of cadherin-based adherens junctions and regulates cell growth and adhesion between cells and anchors the actin cytoskeleton. In some instance, beta-catenin is responsible for transmitting the contact inhibition signal that causes the cells to stop dividing once the epithelial sheet is complete. Beta-catenin is also a key nuclear effector of the Wnt signaling pathway. In some instances, imbalance in the structural and signaling properties of beta-catenin results in diseases and deregulated growth connected to malignancies such as cancer. For example, overexpression of beta-catenin has been linked to cancers such as gastric cancer (Suriano, et al., “Beta-catenin (CTNNB1) gene amplification: a new mechanism of protein overexpression in cancer,” Genes Chromosomes Cancer 42(3): 238-246 (2005)). In some cases, mutations in CTNNB1 gene have been linked to cancer development (e.g., colon cancer, melanoma, hepatocellular carcinoma, ovarian cancer, endometrial cancer, medulloblastoma pilomatricomas, or prostrate cancer), and in some instances, has been linked to metastatic progression. In additional cases, mutations in the CTNNB1 gene cause beta-catenin to translocate to the nucleus without any external stimulus and drive the transcription of its target genes continuously. In some cases, the potential of beta-catenin to change the previously epithelial phenotype of affected cells into an invasive, mesenchyme-like type contributes to metastasis formation.

In some embodiments, CTNNB1 gene is wild type CTNNB1 or CTNNB1 comprising one or more mutations. In some instances, CTNNB1 is wild type CTNNB1. In some instances, CTNNB1 is CTNNB1 comprising one or more mutations. In some instances, the polynucleic acid molecule is a polynucleic acid molecule that hybridizes to a target region of wild type CTNNB1. In some instances, the polynucleic acid molecule is a polynucleic acid molecule that hybridizes to a target region of CTNNB1 comprising a mutation (e.g., a substitution, a deletion, or an addition).

In some embodiments, CTNNB1 DNA or RNA comprises one or more mutations. In some embodiments, CTNNB1 DNA or RNA comprises one or more mutations within one or more exons. In some instances, the one or more exons comprise exon 3. In some instances, CTNNB1 DNA or RNA comprises one or more mutations at codons 32, 33, 34, 37, 41, 45, 183, 245, 287 or a combination thereof. In some instances, CTNNB1 DNA or RNA comprises one or more mutations at positions corresponding to amino acid residues 25, 31, 32, 33, 34, 35, 36, 37, 41, 45, 140, 162, 170, 199, 213, 215, 257, 303, 322, 334, 354, 367, 373, 383, 387, 402, 426, 453, 474, 486, 515, 517, 535, 553, 555, 582, 587, 619, 623, 641, 646, 688, 703, 710, 712, 714, 724, 738, 777, or a combination thereof of the CTNNB1 polypeptide. In some embodiments, CTNNB1 DNA or RNA comprises one or more mutations at positions corresponding to amino acid residues selected from W25 (nonsense mutation), L31M, D32A, D32N, D32Y, D32G, D32H, S33C, S33Y, S33F, S33P, G34R, G34E, G34V, I35S, H36Y, S37F, S37P, S37C, S37A, T41N, T41A, T41I, S45Y, S45F, S45C, I140T, D162E, K170M, V199I, C213F, A215T, T257I, I303M, Q322K, E334K, K354T, G367V, P373S, W383G, N387K, L402F, N426D, R453L, R453Q, R474 (nonsense mutation), R486C, R515Q, L517F, R535 (nonsense mutation), R535Q, M553V, G555A, R582Q, R587Q, C619Y, Q623E, T641 (frame shift), S646F, M688T, Q703H, R710H, D712N, P714R, Y724H, E738K, F777S, or a combination thereof of the CTNNB1 polypeptide.

In some embodiments, the polynucleic acid molecule hybridizes to a target region of CTNNB1 DNA or RNA comprising one or more mutations. In some embodiments, the polynucleic acid molecule hybridizes to a target region of CTNNB1 DNA or RNA comprising one or more mutations within exon 3. In some embodiments, the polynucleic acid molecule hybridizes to a target region of CTNNB1 DNA or RNA comprising one or more mutations at codons 32, 33, 34, 37, 41, 45, 183, 245, 287 or a combination thereof. In some embodiments, the polynucleic acid molecule hybridizes to a target region of CTNNB1 DNA or RNA comprising one or more mutations at positions corresponding to amino acid residues 25, 31, 32, 33, 34, 35, 36, 37, 41, 45, 140, 162, 170, 199, 213, 215, 257, 303, 322, 334, 354, 367, 373, 383, 387, 402, 426, 453, 474, 486, 515, 517, 535, 553, 555, 582, 587, 619, 623, 641, 646, 688, 703, 710, 712, 714, 724, 738, 777, or a combination thereof of the CTNNB1 polypeptide. In some embodiments, the polynucleic acid molecule hybridizes to a target region of CTNNB1 DNA or RNA comprising one or more mutations selected from W25 (nonsense mutation), L31M, D32A, D32N, D32Y, D32G, D32H, S33C, S33Y, S33F, S33P, G34R, G34E, G34V, I35S, H36Y, S37F, S37P, S37C, S37A, T41N, T41A, T41I, S45Y, S45F, S45C, I140T, D162E, K170M, V199I, C213F, A215T, T257I, I303M, Q322K, E334K, K354T, G367V, P373S, W383G, N387K, L402F, N426D, R453L, R453Q, R474 (nonsense mutation), R486C, R515Q, L517F, R535 (nonsense mutation), R535Q, M553V, G555A, R582Q, R587Q, C619Y, Q623E, T641 (frame shift), S646F, M688T, Q703H, R710H, D712N, P714R, Y724H, E738K, F777S, or a combination thereof of the CTNNB1 polypeptide.

In some embodiments, beta-catenin associated genes further comprise PIK3CA, PIK3CB, and MYC. In some embodiments, beta-catenin associated genes further comprise PIK3CA DNA or RNA. PIK3CA (phosphatidylinositol-4,5-bisphosphate 3-kinase, catalytic subunit alpha or p110α protein) is a class i PI 3-kinase catalytic subunit that uses ATP to phosphorylate phosphatidylinositols. In some embodiments, PIK3CA gene is wild type PIK3CA or PIK3CA comprising one or more mutations. In some instances, PIK3CA DNA or RNA is wild type PIK3CA. In some instances, PIK3CA DNA or RNA comprises one or more mutations. In some instances, the polynucleic acid molecule hybridizes to a target region of wild type PIK3CA DNA or RNA. In some instances, the polynucleic acid molecule hybridizes to a target region of PIK3CA DNA or RNA comprising a mutation (e.g., a substitution, a deletion, or an addition).

In some embodiments, PIK3CA DNA or RNA comprises one or more mutations. In some embodiments, PIK3CA DNA or RNA comprises one or more mutation within one or more exons. In some instances, PIK3CA DNA or RNA comprises one or more mutation within exons 9 and/or 20. In some instances, PIK3CA DNA or RNA comprises one or more mutations at positions corresponding to amino acid residues 1, 4, 10-16, 11-18, 11, 12, 38, 39, 65, 72, 75, 79, 81, 83, 88, 90, 93, 102, 103, 103-104, 103-106, 104, 105-108, 106, 106-107, 106-108, 107, 108, 109-112, 110, 111, 113, 115, 137, 170, 258, 272, 279, 320, 328, 335, 342, 344, 345, 350, 357, 359, 363, 364, 365, 366, 378, 398, 401, 417, 420, 447-455, 449, 449-457, 451, 453, 454, 455, 455-460, 463-465, 471, 495, 522, 538, 539, 542, 545, 546, 547, 576, 604, 614, 617, 629, 643, 663, 682, 725, 726, 777, 791, 818, 866, 901, 909, 939, 951, 958, 970, 971, 975, 992, 1004, 1007, 1016, 1017, 1021, 1025, 1029, 1037, 1040, 1043, 1044, 1045, 1047, 1048, 1049, 1052, 1065, 1069, or a combination thereof of the PIK3CA polypeptide. In some embodiments, PIK3CA DNA or RNA comprises one or more mutations at positions corresponding to amino acid residues selected from M1V, R4 (nonsense mutation), L10-M16 (deletion), W11-P18 (deletion), W11L, G12D, R38L, R38H, R38C, R38S, E39K, E39G, E65K, S72G, Q75E, R79M, E81K, E81 (deletion), F83Y, R88Q, C90Y, C90G, R93Q, R93W, 1102 (deletion), E103G, E103-P104 (deletion), E103-G106 (deletion), P104L, V105-R108 (deletion), G106V, G106-N107 (deletion), G106-R108 (deletion), G106R, N107S, R108L, R108H, E109-I112 (deletion), E110 (deletion), K111E, K111R, K111N, K111 (deletion), L113 (deletion), R115L, Q137L, N170S, D258N, Y272 (nonsense mutation), L2791, G320V, W328S, R335G, T342S, V344G, V344M, V344A, N345K, N345I, N345T, D350N, D350G, R357Q, G359R, G363A, G364R, E365K, E365V, P366R, C378R, C378Y, R398H, R401Q, E417K, C420R, C420G, P447-L455 (deletion), P449L, P449-N457 (deletion), G451R, G451V, E453K, E453Q, E453D, D454Y, L455 (frame shift insertion), L455-G460 (deletion), G463-N465 (deletion), P471L, P471A, H495L, H495Y, E522A, D538N, P539R, E542K, E542V, E542G, E542Q, E542A, E545K, E545A, E545G, E545Q, E545D, Q546K, Q546R, Q546P, E547D, S576Y, C604R, F614I, A617W, S629C, Q643H, I663S, Q682 (deletion), D725N, W726K, R777M, E791Q, R818C, L866W, C901F, F909L, D939G, R951C, Q958R, E970K, C971R, R975S, R992P, M1004I, G1007R, F1016C, D1017H, Y1021H, Y1021C, T1025A, T1025S, D1029H, E1037K, M1040V, M1043V, M1043I, N1044K, N1044Y, D1045V, H1047R, H1047L, H1047Y, H1047Q, H1048R, G1049R, T1052K, H1065L, 1069W (nonstop mutation), or a combination thereof of the PIK3CA polypeptide.

In some embodiments, the polynucleic acid molecule hybridizes to a target region of PIK3CA DNA or RNA comprising one or more mutations. In some embodiments, the polynucleic acid molecule hybridizes to a target region of PIK3CA DNA or RNA comprising one or more mutations within an exon. In some embodiments, the polynucleic acid molecule hybridizes to a target region ofPIK3CA DNA or RNA comprising one or more mutations within exon 9 or exon 20. In some embodiments, the polynucleic acid molecule hybridizes to a target region ofPIK3CA DNA or RNA comprising one or more mutations at positions corresponding to amino acid residues 1, 4, 10-16, 11-18, 11, 12, 38, 39, 65, 72, 75, 79, 81, 83, 88, 90, 93, 102, 103, 103-104, 103-106, 104, 105-108, 106, 106-107, 106-108, 107, 108, 109-112, 110, 111, 113, 115, 137, 170, 258, 272, 279, 320, 328, 335, 342, 344, 345, 350, 357, 359, 363, 364, 365, 366, 378, 398, 401, 417, 420, 447-455, 449, 449-457, 451, 453, 454, 455, 455-460, 463-465, 471, 495, 522, 538, 539, 542, 545, 546, 547, 576, 604, 614, 617, 629, 643, 663, 682, 725, 726, 777, 791, 818, 866, 901, 909, 939, 951, 958, 970, 971, 975, 992, 1004, 1007, 1016, 1017, 1021, 1025, 1029, 1037, 1040, 1043, 1044, 1045, 1047, 1048, 1049, 1052, 1065, 1069, or a combination thereof of the PIK3CA polypeptide. In some embodiments, the polynucleic acid molecule is a polynucleic acid molecule that hybridizes to a target region of PIK3CA DNA or RNA comprising one or more mutations at positions corresponding to amino acid residues selected from M1V, R4 (nonsense mutation), L10-M16 (deletion), W11-P18 (deletion), W11L, G12D, R38L, R38H, R38C, R38S, E39K, E39G, E65K, S72G, Q75E, R79M, E81K, E81 (deletion), F83Y, R88Q, C90Y, C90G, R93Q, R93W, 1102 (deletion), E103G, E103-P104 (deletion), E103-G106 (deletion), P104L, V105-R108 (deletion), G106V, G106-N107 (deletion), G106-R108 (deletion), G106R, N107S, R108L, R108H, E109-I112 (deletion), E110 (deletion), K111E, K111R, K111N, K111 (deletion), L113 (deletion), R115L, Q137L, N170S, D258N, Y272 (nonsense mutation), L2791, G320V, W328S, R335G, T342S, V344G, V344M, V344A, N345K, N345I, N345T, D350N, D350G, R357Q, G359R, G363A, G364R, E365K, E365V, P366R, C378R, C378Y, R398H, R401Q, E417K, C420R, C420G, P447-L455 (deletion), P449L, P449-N457 (deletion), G451R, G451V, E453K, E453Q, E453D, D454Y, L455 (frame shift insertion), L455-G460 (deletion), G463-N465 (deletion), P471L, P471A, H495L, H495Y, E522A, D538N, P539R, E542K, E542V, E542G, E542Q, E542A, E545K, E545A, E545G, E545Q, E545D, Q546K, Q546R, Q546P, E547D, S576Y, C604R, F6141, A617W, S629C, Q643H, I663S, Q682 (deletion), D725N, W726K, R777M, E791Q, R818C, L866W, C901F, F909L, D939G, R951C, Q958R, E970K, C971R, R975S, R992P, M1004I, G1007R, F1016C, D1017H, Y1021H, Y1021C, T1025A, T1025S, D1029H, E1037K, M1040V, M1043V, M1043I, N1044K, N1044Y, D1045V, H1047R, H1047L, H1047Y, H1047Q, H1048R, G1049R, T1052K, H1065L, 1069W (nonstop mutation), or a combination thereof of the PIK3CB polypeptide.

In some embodiments, beta-catenin associated genes further comprise PIK3CB. In some embodiments, PIK3CB gene is wild type or comprises one or more mutations. In some instances, PIK3CB DNA or RNA is wild type PIK3CB DNA or RNA. In some instances, PIK3CB DNA or RNA comprises one or more mutations. In some instances, the polynucleic acid molecule hybridizes to a target region of wild type PIK3CB DNA or RNA. In some instances, the polynucleic acid molecule hybridizes to a target region of PIK3CB DNA or RNA comprising a mutation (e.g., a substitution, a deletion, or an addition).

In some embodiments, PIK3CB DNA or RNA comprises one or more mutations. In some embodiments, PIK3CB DNA or RNA comprises one or more mutations within one or more exons. In some instances, PIK3CB DNA or RNA comprises one or more mutations at positions corresponding to amino acid residues 18, 19, 21, 28, 50, 61, 68, 103, 135, 140, 167, 252, 270, 290, 301, 304, 321, 369, 417, 442, 470, 497, 507, 512, 540, 551, 552, 554, 562, 567, 593, 595, 619, 628, 668, 768, 805, 824, 830, 887, 967, 992, 1005, 1020, 1036, 1046, 1047, 1048, 1049, 1051, 1055, 1067, or a combination thereof of the PIK3CB polypeptide. In some embodiments, PIK3CB DNA or RNA comprises one or more mutations at positions corresponding to amino acid residues selected from W18 (nonsense mutation), A19V, D21H, G28S, A50P, K61T, M681, R103K, H135N, L140S, S167C, G252W, R270W, K290N, E301V, I304R, R321Q, V369I, T417M, N442K, E470K, E497D, P507S, I512M, E540 (nonsense mutation), C551R, E552K, E554K, R562 (nonsense mutation), E567D, A593V, L595P, V619A, R628 (nonsense mutation), R668W, L768F, K805E, D824E, A830T, E887 (nonsense mutation), V967A, I992T, A1005V, D1020H, E1036K, D1046N, E1047K, A1048V, L1049R, E1051K, T1055A, D1067V, D1067A, or a combination thereof of the PIK3CB polypeptide.

In some embodiments, the polynucleic acid molecule hybridizes to a target region of PIK3CB DNA or RNA comprising one or more mutations. In some embodiments, the polynucleic acid molecule hybridizes to a target region of PIK3CB DNA or RNA comprising one or more mutations within an exon. In some embodiments, the polynucleic acid molecule hybridizes to a target region of PIK3CB DNA or RNA comprising one or more mutations at positions corresponding to amino acid residues 18, 19, 21, 28, 50, 61, 68, 103, 135, 140, 167, 252, 270, 290, 301, 304, 321, 369, 417, 442, 470, 497, 507, 512, 540, 551, 552, 554, 562, 567, 593, 595, 619, 628, 668, 768, 805, 824, 830, 887, 967, 992, 1005, 1020, 1036, 1046, 1047, 1048, 1049, 1051, 1055, 1067, or a combination thereof of the PIK3CB polypeptide. In some embodiments, the polynucleic acid molecule hybridizes to a target region of PIK3CB DNA or RNA comprising one or more mutations at positions corresponding to amino acid residues selected from W18 (nonsense mutation), A19V, D21H, G28S, A50P, K61T, M681, R103K, H135N, L140S, S167C, G252W, R270W, K290N, E301V, I304R, R321Q, V369I, T417M, N442K, E470K, E497D, P507S, I512M, E540 (nonsense mutation), C551R, E552K, E554K, R562 (nonsense mutation), E567D, A593V, L595P, V619A, R628 (nonsense mutation), R668W, L768F, K805E, D824E, A830T, E887 (nonsense mutation), V967A, I992T, A1005V, D1020H, E1036K, D1046N, E1047K, A1048V, L1049R, E1051K, T1055A, D1067V, D1067A, or a combination thereof of the PIK3CB polypeptide.

In some embodiments, beta-catenin associated genes further comprise MYC. In some embodiments, MYC gene is wild type MYC or MYC comprising one or more mutations. In some instances, MYC is wild type MYC DNA or RNA. In some instances, MYC DNA or RNA comprises one or more mutations. In some instances, the polynucleic acid molecule hybridizes to a target region of wild type MYC DNA or RNA. In some instances, the polynucleic acid molecule is a polynucleic acid molecule that hybridizes to a target region of MYC DNA or RNA comprising a mutation (e.g., a substitution, a deletion, or an addition).

In some embodiments, MYC DNA or RNA comprises one or more mutations. In some embodiments, MYC DNA or RNA comprises one or more mutation within one or more exons. In some instances, MYC DNA or RNA comprises one or more mutations within exon 2 or exon 3. In some instances, MYC DNA or RNA comprises one or more mutations at positions corresponding to amino acid residues 2, 7, 17, 20, 32, 44, 58, 59, 76, 115, 138, 141, 145, 146, 169, 175, 188, 200, 202, 203, 248, 251, 298, 321, 340, 369, 373, 374, 389, 395, 404, 419, 431, 439, or a combination thereof. In some embodiments, MYC DNA or RNA comprises one or more mutations at positions corresponding to amino acid residues selected from P2L, F7L, D17N, Q20E, Y32N, A44V, A44T, T58I, P59L, A76V, F115L, F138S, A141S, V145I, S146L, S169C, S175N, C188F, N200S, S202N, S203T, T248S, D251E, S298Y, Q321E, V340D, V369D, T373K, H374R, F389L, Q395H, K404N, L419M, E431K, R439Q, or a combination thereof of the MYC polypeptide.

In some embodiments, the polynucleic acid molecule hybridizes to a target region of MYC DNA or RNA comprising one or more mutations. In some embodiments, the polynucleic acid molecule hybridizes to a target region of MYC DNA or RNA comprising one or more mutations within an exon. In some embodiments, the polynucleic acid molecule hybridizes to a target region of MYC DNA or RNA comprising one or more mutations within exon 2 or exon 3. In some embodiments, the polynucleic acid molecule hybridizes to a target region of MYC DNA or RNA comprising one or more mutations at positions corresponding to amino acid residues 2, 7, 17, 20, 32, 44, 58, 59, 76, 115, 138, 141, 145, 146, 169, 175, 188, 200, 202, 203, 248, 251, 298, 321, 340, 369, 373, 374, 389, 395, 404, 419, 431, 439, or a combination thereof of the MYC polypeptide. In some embodiments, the polynucleic acid molecule hybridizes to a target region of MYC DNA or RNA comprising one or more mutations at positions corresponding to amino acid residues selected from P2L, F7L, D17N, Q20E, Y32N, A44V, A44T, T581, P59L, A76V, F115L, F138S, A141S, V145I, S146L, S169C, S175N, C188F, N200S, S202N, S203T, T248S, D251E, S298Y, Q321E, V340D, V369D, T373K, H374R, F389L, Q395H, K404N, L419M, E431K, R439Q, or a combination thereof of the MYC polypeptide.

### Polynucleic Acid Molecules That Target Hypoxanthine Phosphoribosyltransferase 1 (HPRT1)

Hypoxanthine-guanine phosphoribosyltransferase (HGPRT) is a transferase that catalyzes the conversion of hypoxanthine to inosine monophosphate and guanine to guanosine monophosphate. HGPRT is encoded by the hypoxanthine Phosphoribosyltransferase 1 (HPRT1) gene.

In some embodiments, HPRT1 DNA or RNA is wild type or comprises one or more mutations. In some instances, HPRT1 DNA or RNA comprises one or more mutations within one or more exons. In some instances, the one or more exons comprise exon 2, exon 3, exon 4, exon 6, exon 8, or exon 9. In some instances, HPRT1 DNA or RNA comprises one or more mutations at positions corresponding to amino acid residues 35, 48, 56, 74, 87, 129, 154, 162, 195, 200, 210, or a combination thereof of the HPRT1 polypeptide. In some embodiments, the polynucleic acid molecule hybridizes to a target region of HPRT1 DNA or RNA comprising one or more mutations selected from V35M, R48H, E56D, F74L, R87I, N129 (splice-site mutation), N154H, S162 (splice-site mutation), Y195C, Y195N, R200M, E210K, or a combination thereof of the HPRT1 polypeptide.

In some embodiments, the polynucleic acid molecule hybridizes to a target region of HPRT1 DNA or RNA comprising one or more mutations. In some embodiments, the polynucleic acid molecule hybridizes to a target region of HPRT1 DNA or RNA comprising one or more mutations within exon 2, exon 3, exon 4, exon 6, exon 8, or exon 9. In some embodiments, the polynucleic acid molecule hybridizes to a target region of HPRT1 DNA or RNA comprising one or more mutations at positions corresponding to amino acid residues 35, 48, 56, 74, 87, 129, 154, 162, 195, 200, 210, or a combination thereof of the HPRT1 polypeptide. In some embodiments, the polynucleic acid molecule hybridizes to a target region of HPRT1 DNA or RNA comprising one or more mutations selected from V35M, R48H, E56D, F74L, R871, N129 (splice-site mutation), N154H, S162 (splice-site mutation), Y195C, Y195N, R200M, E210K, or a combination thereof of the HPRT1 polypeptide.

### Polynucleic Acid Molecule Sequences

In some embodiments, the polynucleic acid molecule comprises a sequence that hybridizes to a target sequence illustrated in Tables 1, 3, 5, 6, or 7. In some instances, the polynucleic acid molecule is B. In some instances, the polynucleic acid molecule B comprises a sequence that hybridizes to a target sequence illustrated in Table 1 (KRAS target sequences). In some instances, the polynucleic acid molecule B comprises a sequence that hybridizes to a target sequence illustrated in Table 3 (EGFR target sequences). In some cases, the polynucleic acid molecule B comprises a sequence that hybridizes to a target sequence illustrated in Table 5 (AR target sequences). In some cases, the polynucleic acid molecule B comprises a sequence that hybridizes to a target sequence illustrated in Table 6 (β-catenin target sequences). In additional cases, the polynucleic acid molecule B comprises a sequence that hybridizes to a target sequence illustrated in Table 7 (PIK3CA and PIK3CB target sequences).

In some embodiments, the polynucleic acid molecule B comprises a sequence having at least 50%, 55%, 60%, 65%, 70%, 75%, 80%, 85%, 90%, 95%, 96%, 97%, 98%, 99%, or 100% sequence identity to a sequence listed in Table 2. In some embodiments, the polynucleic acid molecule comprises a sequence having at least 50%, 55%, 60%, 65%, 70%, 75%, 80%, 85%, 90%, 95%, 96%, 97%, 98%, 99%, or 100% sequence identity to SEQ ID NOs: 16-45. In some embodiments, the polynucleic acid molecule comprises a sequence having at least 50% sequence identity to SEQ ID NOs: 16-45. In some embodiments, the polynucleic acid molecule comprises a sequence having at least 60% sequence identity to SEQ ID NOs: 16-45. In some embodiments, the polynucleic acid molecule comprises a sequence having at least 70% sequence identity to SEQ ID NOs: 16-45. In some embodiments, the polynucleic acid molecule comprises a sequence having at least 75% sequence identity to SEQ ID NOs: 16-45. In some embodiments, the polynucleic acid molecule comprises a sequence having at least 80% sequence identity to SEQ ID NOs: 16-45. In some embodiments, the polynucleic acid molecule comprises a sequence having at least 85% sequence identity to SEQ ID NOs: 16-45. In some embodiments, the polynucleic acid molecule comprises a sequence having at least 90% sequence identity to SEQ ID NOs: 16-45. In some embodiments, the polynucleic acid molecule comprises a sequence having at least 95% sequence identity to SEQ ID NOs: 16-45. In some embodiments, the polynucleic acid molecule comprises a sequence having at least 96% sequence identity to SEQ ID NOs: 16-45. In some embodiments, the polynucleic acid molecule comprises a sequence having at least 97% sequence identity to SEQ ID NOs: 16-45. In some embodiments, the polynucleic acid molecule comprises a sequence having at least 98% sequence identity to SEQ ID NOs: 16-45. In some embodiments, the polynucleic acid molecule comprises a sequence having at least 99% sequence identity to SEQ ID NOs: 16-45. In some embodiments, the polynucleic acid molecule consists of SEQ ID NOs: 16-45.

In some embodiments, the polynucleic acid molecule B comprises a first polynucleotide and a second polynucleotide. In some instances, the first polynucleotide comprises a sequence having at least 50%, 55%, 60%, 65%, 70%, 75%, 80%, 85%, 90%, 95%, 96%, 97%, 98%, 99%, or 100% sequence identity to SEQ ID NOs: 16-45. In some cases, the second polynucleotide comprises a sequence having at least 50%, 55%, 60%, 65%, 70%, 75%, 80%, 85%, 90%, 95%, 96%, 97%, 98%, 99%, or 100% sequence identity to SEQ ID NOs: 16-45. In some cases, the polynucleic acid molecule comprises a first polynucleotide having at least 50%, 55%, 60%, 65%, 70%, 75%, 80%, 85%, 90%, 95%, 96%, 97%, 98%, 99%, or 100% sequence identity to SEQ ID NOs: 16-45 and a second polynucleotide having at least 50%, 55%, 60%, 65%, 70%, 75%, 80%, 85%, 90%, 95%, 96%, 97%, 98%, 99%, or 100% sequence identity to SEQ ID NOs: 16-45.

In some embodiments, the polynucleic acid molecule B comprises a sequence having at least 50%, 55%, 60%, 65%, 70%, 75%, 80%, 85%, 90%, 95%, 96%, 97%, 98%, 99%, or 100% sequence identity to a sequence listed in Table 4. In some embodiments, the polynucleic acid molecule comprises a sequence having at least 50%, 55%, 60%, 65%, 70%, 75%, 80%, 85%, 90%, 95%, 96%, 97%, 98%, 99%, or 100% sequence identity to SEQ ID NOs: 422-1173. In some embodiments, the polynucleic acid molecule comprises a sequence having at least 50% sequence identity to SEQ ID NOs: 422-1173. In some embodiments, the polynucleic acid molecule comprises a sequence having at least 60% sequence identity to SEQ ID NOs: 422-1173. In some embodiments, the polynucleic acid molecule comprises a sequence having at least 70% sequence identity to SEQ ID NOs: 422-1173. In some embodiments, the polynucleic acid molecule comprises a sequence having at least 75% sequence identity to SEQ ID NOs: 422-1173. In some embodiments, the polynucleic acid molecule comprises a sequence having at least 80% sequence identity to SEQ ID NOs: 422-1173. In some embodiments, the polynucleic acid molecule comprises a sequence having at least 85% sequence identity to SEQ ID NOs: 422-1173. In some embodiments, the polynucleic acid molecule comprises a sequence having at least 90% sequence identity to SEQ ID NOs: 422-1173. In some embodiments, the polynucleic acid molecule comprises a sequence having at least 95% sequence identity to SEQ ID NOs: 422-1173. In some embodiments, the polynucleic acid molecule comprises a sequence having at least 96% sequence identity to SEQ ID NOs: 422-1173. In some embodiments, the polynucleic acid molecule comprises a sequence having at least 97% sequence identity to SEQ ID NOs: 422-1173. In some embodiments, the polynucleic acid molecule comprises a sequence having at least 98% sequence identity to SEQ ID NOs: 422-1173. In some embodiments, the polynucleic acid molecule comprises a sequence having at least 99% sequence identity to SEQ ID NOs: 422-1173. In some embodiments, the polynucleic acid molecule consists of SEQ ID NOs: 422-1173.

In some embodiments, the polynucleic acid molecule B comprises a first polynucleotide and a second polynucleotide. In some instances, the first polynucleotide comprises a sequence having at least 50%, 55%, 60%, 65%, 70%, 75%, 80%, 85%, 90%, 95%, 96%, 97%, 98%, 99%, or 100% sequence identity to SEQ ID NOs: 422-1173. In some cases, the second polynucleotide comprises a sequence having at least 50%, 55%, 60%, 65%, 70%, 75%, 80%, 85%, 90%, 95%, 96%, 97%, 98%, 99%, or 100% sequence identity to SEQ ID NOs: 422-1173. In some cases, the polynucleic acid molecule comprises a first polynucleotide having at least 50%, 55%, 60%, 65%, 70%, 75%, 80%, 85%, 90%, 95%, 96%, 97%, 98%, 99%, or 100% sequence identity to SEQ ID NOs: 422-1173 and a second polynucleotide having at least 50%, 55%, 60%, 65%, 70%, 75%, 80%, 85%, 90%, 95%, 96%, 97%, 98%, 99%, or 100% sequence identity to SEQ ID NOs: 422-1173.

In some embodiments, the polynucleic acid molecule B comprises a sequence having at least 50%, 55%, 60%, 65%, 70%, 75%, 80%, 85%, 90%, 95%, 96%, 97%, 98%, 99%, or 100% sequence identity to a sequence listed in Table 8. In some embodiments, the polynucleic acid molecule comprises a sequence having at least 50%, 55%, 60%, 65%, 70%, 75%, 80%, 85%, 90%, 95%, 96%, 97%, 98%, 99%, or 100% sequence identity to SEQ ID NOs: 1195-1214. In some embodiments, the polynucleic acid molecule comprises a sequence having at least 50% sequence identity to SEQ ID NOs: 1195-1214. In some embodiments, the polynucleic acid molecule comprises a sequence having at least 60% sequence identity to SEQ ID NOs: 1195-1214. In some embodiments, the polynucleic acid molecule comprises a sequence having at least 70% sequence identity to SEQ ID NOs: 1195-1214. In some embodiments, the polynucleic acid molecule comprises a sequence having at least 75% sequence identity to SEQ ID NOs: 1195-1214. In some embodiments, the polynucleic acid molecule comprises a sequence having at least 80% sequence identity to SEQ ID NOs: 1195-1214. In some embodiments, the polynucleic acid molecule comprises a sequence having at least 85% sequence identity to SEQ ID NOs: 1195-1214. In some embodiments, the polynucleic acid molecule comprises a sequence having at least 90% sequence identity to SEQ ID NOs: 1195-1214. In some embodiments, the polynucleic acid molecule comprises a sequence having at least 95% sequence identity to SEQ ID NOs: 1195-1214. In some embodiments, the polynucleic acid molecule comprises a sequence having at least 96% sequence identity to SEQ ID NOs: 1195-1214. In some embodiments, the polynucleic acid molecule comprises a sequence having at least 97% sequence identity to SEQ ID NOs: 1195-1214. In some embodiments, the polynucleic acid molecule comprises a sequence having at least 98% sequence identity to SEQ ID NOs: 1195-1214. In some embodiments, the polynucleic acid molecule comprises a sequence having at least 99% sequence identity to SEQ ID NOs: 1195-1214. In some embodiments, the polynucleic acid molecule consists of SEQ ID NOs: 1195-1214.

In some embodiments, the polynucleic acid molecule B comprises a first polynucleotide and a second polynucleotide. In some instances, the first polynucleotide comprises a sequence having at least 50%, 55%, 60%, 65%, 70%, 75%, 80%, 85%, 90%, 95%, 96%, 97%, 98%, 99%, or 100% sequence identity to SEQ ID NOs: 1195-1214. In some cases, the second polynucleotide comprises a sequence that is complementary to a sequence having at least 50%, 55%, 60%, 65%, 70%, 75%, 80%, 85%, 90%, 95%, 96%, 97%, 98%, 99%, or 100% sequence identity to SEQ ID NOs: 1195-1214. In some instances, the polynucleic acid molecule comprises a first polynucleotide having at least 50%, 55%, 60%, 65%, 70%, 75%, 80%, 85%, 90%, 95%, 96%, 97%, 98%, 99%, or 100% sequence identity to SEQ ID NOs: 1195-1214, and a second polynucleotide that is complementary to a sequence having at least 50%, 55%, 60%, 65%, 70%, 75%, 80%, 85%, 90%, 95%, 96%, 97%, 98%, 99%, or 100% sequence identity to SEQ ID NOs: 1195-1214.

In some embodiments, the polynucleic acid molecule B comprises a sequence having at least 50%, 55%, 60%, 65%, 70%, 75%, 80%, 85%, 90%, 95%, 96%, 97%, 98%, 99%, or 100% sequence identity to a sequence listed in Table 9. In some embodiments, the polynucleic acid molecule comprises a sequence having at least 50%, 55%, 60%, 65%, 70%, 75%, 80%, 85%, 90%, 95%, 96%, 97%, 98%, 99%, or 100% sequence identity to SEQ ID NOs: 1215-1242. In some embodiments, the polynucleic acid molecule comprises a sequence having at least 50% sequence identity to SEQ ID NOs: 1215-1242. In some embodiments, the polynucleic acid molecule comprises a sequence having at least 60% sequence identity to SEQ ID NOs: 1215-1242. In some embodiments, the polynucleic acid molecule comprises a sequence having at least 70% sequence identity to SEQ ID NOs: 1215-1242. In some embodiments, the polynucleic acid molecule comprises a sequence having at least 75% sequence identity to SEQ ID NOs: 1215-1242. In some embodiments, the polynucleic acid molecule comprises a sequence having at least 80% sequence identity to SEQ ID NOs: 1215-1242. In some embodiments, the polynucleic acid molecule comprises a sequence having at least 85% sequence identity to SEQ ID NOs: 1215-1242. In some embodiments, the polynucleic acid molecule comprises a sequence having at least 90% sequence identity to SEQ ID NOs: 1215-1242. In some embodiments, the polynucleic acid molecule comprises a sequence having at least 95% sequence identity to SEQ ID NOs: 1215-1242. In some embodiments, the polynucleic acid molecule comprises a sequence having at least 96% sequence identity to SEQ ID NOs: 1215-1242. In some embodiments, the polynucleic acid molecule comprises a sequence having at least 97% sequence identity to SEQ ID NOs: 1215-1242. In some embodiments, the polynucleic acid molecule comprises a sequence having at least 98% sequence identity to SEQ ID NOs: 1215-1242. In some embodiments, the polynucleic acid molecule comprises a sequence having at least 99% sequence identity to SEQ ID NOs: 1215-1242. In some embodiments, the polynucleic acid molecule consists of SEQ ID NOs: 1215-1242.

In some embodiments, the polynucleic acid molecule B comprises a first polynucleotide and a second polynucleotide. In some instances, the first polynucleotide comprises a sequence having at least 50%, 55%, 60%, 65%, 70%, 75%, 80%, 85%, 90%, 95%, 96%, 97%, 98%, 99%, or 100% sequence identity to SEQ ID NOs: 1215-1242. In some cases, the second polynucleotide comprises a sequence having at least 50%, 55%, 60%, 65%, 70%, 75%, 80%, 85%, 90%, 95%, 96%, 97%, 98%, 99%, or 100% sequence identity to SEQ ID NOs: 1215-1242. In some cases, the polynucleic acid molecule comprises a first polynucleotide having at least 50%, 55%, 60%, 65%, 70%, 75%, 80%, 85%, 90%, 95%, 96%, 97%, 98%, 99%, or 100% sequence identity to SEQ ID NOs: 1215-1242 and a second polynucleotide having at least 50%, 55%, 60%, 65%, 70%, 75%, 80%, 85%, 90%, 95%, 96%, 97%, 98%, 99%, or 100% sequence identity to SEQ ID NOs: 1215-1242.

### Polynucleic Acid Molecules

In some embodiments, the polynucleic acid molecule described herein comprises RNA or DNA. In some cases, the polynucleic acid molecule comprises RNA. In some instances, RNA comprises short interfering RNA (siRNA), short hairpin RNA (shRNA), microRNA (miRNA), double-stranded RNA (dsRNA), transfer RNA (tRNA), ribosomal RNA (rRNA), or heterogeneous nuclear RNA (hnRNA). In some instances, RNA comprises shRNA. In some instances, RNA comprises miRNA. In some instances, RNA comprises dsRNA. In some instances, RNA comprises tRNA. In some instances, RNA comprises rRNA. In some instances, RNA comprises hnRNA. In some instances, the RNA comprises siRNA. In some instances, the polynucleic acid molecule comprises siRNA. In some cases, B comprises siRNA.

In some embodiments, the polynucleic acid molecule is from about 10 to about 50 nucleotides in length. In some instances, the polynucleic acid molecule is from about 10 to about 30, from about 15 to about 30, from about 18 to about 25, from about 18 to about 24, from about 19 to about 23, or from about 20 to about 22 nucleotides in length.

In some embodiments, the polynucleic acid molecule is about 50 nucleotides in length. In some instances, the polynucleic acid molecule is about 45 nucleotides in length. In some instances, the polynucleic acid molecule is about 40 nucleotides in length. In some instances, the polynucleic acid molecule is about 35 nucleotides in length. In some instances, the polynucleic acid molecule is about 30 nucleotides in length. In some instances, the polynucleic acid molecule is about 25 nucleotides in length. In some instances, the polynucleic acid molecule is about 20 nucleotides in length. In some instances, the polynucleic acid molecule is about 19 nucleotides in length. In some instances, the polynucleic acid molecule is about 18 nucleotides in length. In some instances, the polynucleic acid molecule is about 17 nucleotides in length. In some instances, the polynucleic acid molecule is about 16 nucleotides in length. In some instances, the polynucleic acid molecule is about 15 nucleotides in length. In some instances, the polynucleic acid molecule is about 14 nucleotides in length. In some instances, the polynucleic acid molecule is about 13 nucleotides in length. In some instances, the polynucleic acid molecule is about 12 nucleotides in length. In some instances, the polynucleic acid molecule is about 11 nucleotides in length. In some instances, the polynucleic acid molecule is about 10 nucleotides in length. In some instances, the polynucleic acid molecule is from about 10 to about 50 nucleotides in length. In some instances, the polynucleic acid molecule is from about 10 to about 45 nucleotides in length. In some instances, the polynucleic acid molecule is from about 10 to about 40 nucleotides in length. In some instances, the polynucleic acid molecule is from about 10 to about 35 nucleotides in length. In some instances, the polynucleic acid molecule is from about 10 to about 30 nucleotides in length. In some instances, the polynucleic acid molecule is from about 10 to about 25 nucleotides in length. In some instances, the polynucleic acid molecule is from about 10 to about 20 nucleotides in length. In some instances, the polynucleic acid molecule is from about 15 to about 25 nucleotides in length. In some instances, the polynucleic acid molecule is from about 15 to about 30 nucleotides in length. In some instances, the polynucleic acid molecule is from about 12 to about 30 nucleotides in length.

In some embodiments, the polynucleic acid molecule comprises a first polynucleotide. In some instances, the polynucleic acid molecule comprises a second polynucleotide. In some instances, the polynucleic acid molecule comprises a first polynucleotide and a second polynucleotide. In some instances, the first polynucleotide is a sense strand or passenger strand. In some instances, the second polynucleotide is an antisense strand or guide strand.

In some embodiments, the polynucleic acid molecule is a first polynucleotide. In some embodiments, the first polynucleotide is from about 10 to about 50 nucleotides in length. In some instances, the first polynucleotide is from about 10 to about 30, from about 15 to about 30, from about 18 to about 25, from about 18 to about 24, from about 19 to about 23, or from about 20 to about 22 nucleotides in length.

In some instances, the first polynucleotide is about 50 nucleotides in length. In some instances, the first polynucleotide is about 45 nucleotides in length. In some instances, the first polynucleotide is about 40 nucleotides in length. In some instances, the first polynucleotide is about 35 nucleotides in length. In some instances, the first polynucleotide is about 30 nucleotides in length. In some instances, the first polynucleotide is about 25 nucleotides in length. In some instances, the first polynucleotide is about 20 nucleotides in length. In some instances, the first polynucleotide is about 19 nucleotides in length. In some instances, the first polynucleotide is about 18 nucleotides in length. In some instances, the first polynucleotide is about 17 nucleotides in length. In some instances, the first polynucleotide is about 16 nucleotides in length. In some instances, the first polynucleotide is about 15 nucleotides in length. In some instances, the first polynucleotide is about 14 nucleotides in length. In some instances, the first polynucleotide is about 13 nucleotides in length. In some instances, the first polynucleotide is about 12 nucleotides in length. In some instances, the first polynucleotide is about 11 nucleotides in length. In some instances, the first polynucleotide is about 10 nucleotides in length. In some instances, the first polynucleotide is from about 10 to about 50 nucleotides in length. In some instances, the first polynucleotide is from about 10 to about 45 nucleotides in length. In some instances, the first polynucleotide is from about 10 to about 40 nucleotides in length. In some instances, the first polynucleotide is from about 10 to about 35 nucleotides in length. In some instances, the first polynucleotide is from about 10 to about 30 nucleotides in length. In some instances, the first polynucleotide is from about 10 to about 25 nucleotides in length. In some instances, the first polynucleotide is from about 10 to about 20 nucleotides in length. In some instances, the first polynucleotide is from about 15 to about 25 nucleotides in length. In some instances, the first polynucleotide is from about 15 to about 30 nucleotides in length. In some instances, the first polynucleotide is from about 12 to about 30 nucleotides in length.

In some embodiments, the polynucleic acid molecule is a second polynucleotide. In some embodiments, the second polynucleotide is from about 10 to about 50 nucleotides in length. In some instances, the second polynucleotide is from about 10 to about 30, from about 15 to about 30, from about 18 to about 25, from about 18 to about 24, from about 19 to about 23, or from about 20 to about 22 nucleotides in length.

In some instances, the second polynucleotide is about 50 nucleotides in length. In some instances, the second polynucleotide is about 45 nucleotides in length. In some instances, the second polynucleotide is about 40 nucleotides in length. In some instances, the second polynucleotide is about 35 nucleotides in length. In some instances, the second polynucleotide is about 30 nucleotides in length. In some instances, the second polynucleotide is about 25 nucleotides in length. In some instances, the second polynucleotide is about 20 nucleotides in length. In some instances, the second polynucleotide is about 19 nucleotides in length. In some instances, the second polynucleotide is about 18 nucleotides in length. In some instances, the second polynucleotide is about 17 nucleotides in length. In some instances, the second polynucleotide is about 16 nucleotides in length. In some instances, the second polynucleotide is about 15 nucleotides in length. In some instances, the second polynucleotide is about 14 nucleotides in length. In some instances, the second polynucleotide is about 13 nucleotides in length. In some instances, the second polynucleotide is about 12 nucleotides in length. In some instances, the second polynucleotide is about 11 nucleotides in length. In some instances, the second polynucleotide is about 10 nucleotides in length. In some instances, the second polynucleotide is from about 10 to about 50 nucleotides in length. In some instances, the second polynucleotide is from about 10 to about 45 nucleotides in length. In some instances, the second polynucleotide is from about 10 to about 40 nucleotides in length. In some instances, the second polynucleotide is from about 10 to about 35 nucleotides in length. In some instances, the second polynucleotide is from about 10 to about 30 nucleotides in length. In some instances, the second polynucleotide is from about 10 to about 25 nucleotides in length. In some instances, the second polynucleotide is from about 10 to about 20 nucleotides in length. In some instances, the second polynucleotide is from about 15 to about 25 nucleotides in length. In some instances, the second polynucleotide is from about 15 to about 30 nucleotides in length. In some instances, the second polynucleotide is from about 12 to about 30 nucleotides in length.

In some embodiments, the polynucleic acid molecule comprises a first polynucleotide and a second polynucleotide. In some instances, the polynucleic acid molecule further comprises a blunt terminus, an overhang, or a combination thereof. In some instances, the blunt terminus is a 5′ blunt terminus, a 3′ blunt terminus, or both. In some cases, the overhang is a 5′ overhang, 3′ overhang, or both. In some cases, the overhang comprises 1, 2, 3, 4, 5, 6, 7, 8, 9, or 10 non-base pairing nucleotides. In some cases, the overhang comprises 1, 2, 3, 4, 5, or 6 non-base pairing nucleotides. In some cases, the overhang comprises 1, 2, 3, or 4 non-base pairing nucleotides. In some cases, the overhang comprises 1 non-base pairing nucleotide. In some cases, the overhang comprises 2 non-base pairing nucleotides. In some cases, the overhang comprises 3 non-base pairing nucleotides. In some cases, the overhang comprises 4 non-base pairing nucleotides.

In some embodiments, the sequence of the polynucleic acid molecule is at least 40%, 50%, 55%, 60%, 65%, 70%, 75%, 80%, 85%, 90%, 95%, 98%, 99%, or 99.5% complementary to a target sequence described herein. In some embodiments, the sequence of the polynucleic acid molecule is at least 50% complementary to a target sequence described herein. In some embodiments, the sequence of the polynucleic acid molecule is at least 60% complementary to a target sequence described herein. In some embodiments, the sequence of the polynucleic acid molecule is at least 70% complementary to a target sequence described herein. In some embodiments, the sequence of the polynucleic acid molecule is at least 80% complementary to a target sequence described herein. In some embodiments, the sequence of the polynucleic acid molecule is at least 90% complementary to a target sequence described herein. In some embodiments, the sequence of the polynucleic acid molecule is at least 95% complementary to a target sequence described herein. In some embodiments, the sequence of the polynucleic acid molecule is at least 99% complementary to a target sequence described herein. In some instances, the sequence of the polynucleic acid molecule is 100% complementary to a target sequence described herein.

In some embodiments, the sequence of the polynucleic acid molecule has 5 or less mismatches to a target sequence described herein. In some embodiments, the sequence of the polynucleic acid molecule has 4 or less mismatches to a target sequence described herein. In some instances, the sequence of the polynucleic acid molecule may has 3 or less mismatches to a target sequence described herein. In some cases, the sequence of the polynucleic acid molecule may has 2 or less mismatches to a target sequence described herein. In some cases, the sequence of the polynucleic acid molecule may has 1 or less mismatches to a target sequence described herein.

In some embodiments, the specificity of the polynucleic acid molecule that hybridizes to a target sequence described herein is a 95%, 98%, 99%, 99.5%, or 100% sequence complementarity of the polynucleic acid molecule to a target sequence. In some instances, the hybridization is a high stringent hybridization condition.

In some embodiments, the polynucleic acid molecule hybridizes to at least 8, 9, 10, 11, 12, 13, 14, 15, 16, 17, 18, 19, 20, or more contiguous bases of a target sequence described herein. In some embodiments, the polynucleic acid molecule hybridizes to at least 8 contiguous bases of a target sequence described herein. In some embodiments, the polynucleic acid molecule hybridizes to at least 9 contiguous bases of a target sequence described herein. In some embodiments, the polynucleic acid molecule hybridizes to at least 10 contiguous bases of a target sequence described herein. In some embodiments, the polynucleic acid molecule hybridizes to at least 11 contiguous bases of a target sequence described herein. In some embodiments, the polynucleic acid molecule hybridizes to at least 12 contiguous bases of a target sequence described herein. In some embodiments, the polynucleic acid molecule hybridizes to at least 13 contiguous bases of a target sequence described herein. In some embodiments, the polynucleic acid molecule hybridizes to at least 14 contiguous bases of a target sequence described herein. In some embodiments, the polynucleic acid molecule hybridizes to at least 15 contiguous bases of a target sequence described herein. In some embodiments, the polynucleic acid molecule hybridizes to at least 16 contiguous bases of a target sequence described herein. In some embodiments, the polynucleic acid molecule hybridizes to at least 17 contiguous bases of a target sequence described herein. In some embodiments, the polynucleic acid molecule hybridizes to at least 18 contiguous bases of a target sequence described herein. In some embodiments, the polynucleic acid molecule hybridizes to at least 19 contiguous bases of a target sequence described herein. In some embodiments, the polynucleic acid molecule hybridizes to at least 20 contiguous bases of a target sequence described herein.

In some embodiments, the polynucleic acid molecule has reduced off-target effect. In some instances, “off-target” or “off-target effects” refer to any instance in which a polynucleic acid polymer directed against a given target causes an unintended effect by interacting either directly or indirectly with another mRNA sequence, a DNA sequence or a cellular protein or other moiety. In some instances, an “off-target effect” occurs when there is a simultaneous degradation of other transcripts due to partial homology or complementarity between that other transcript and the sense and/or antisense strand of the polynucleic acid molecule.

In some embodiments, the polynucleic acid molecule comprises natural, synthetic, or artificial nucleotide analogues or bases. In some cases, the polynucleic acid molecule comprises combinations of DNA, RNA and/or nucleotide analogues. In some instances, the synthetic or artificial nucleotide analogues or bases comprise modifications at one or more of ribose moiety, phosphate moiety, nucleoside moiety, or a combination thereof.

In some embodiments, a nucleotide analogue or artificial nucleotide base described above comprises a 5′-vinylphosphonate modified nucleotide nucleic acid with a modification at a 5′ hydroxyl group of the ribose moiety. In some embodiments, the 5′-vinylphosphonate modified nucleotide is selected from the nucleotide provided below.

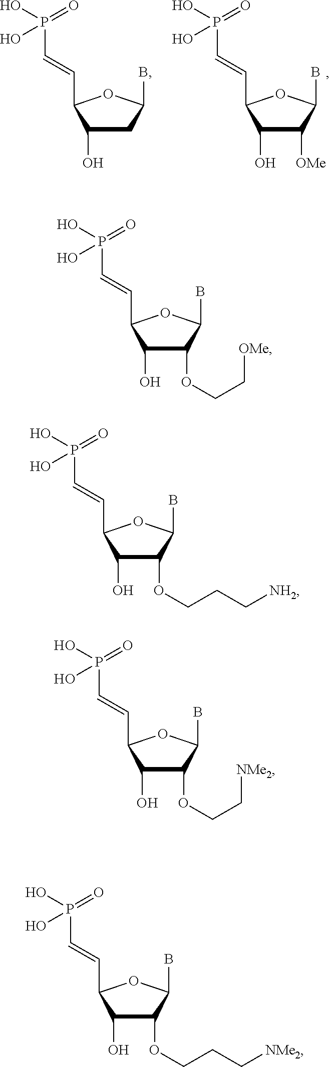

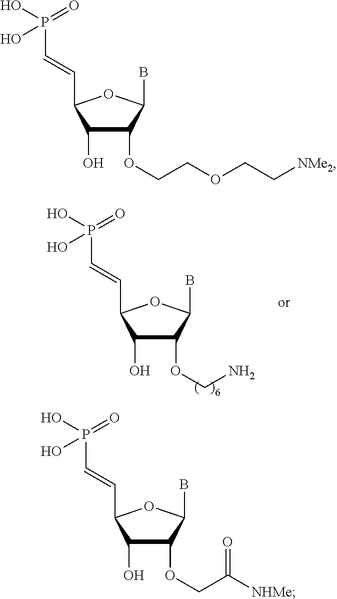

In some instances, the modification at the 2′ hydroxyl group is a 2′-O-aminopropyl modification in which an extended amine group comprising a propyl linker binds the amine group to the 2′ oxygen. In some instances, this modification neutralizes the phosphate-derived overall negative charge of the oligonucleotide molecule by introducing one positive charge from the amine group per sugar and thereby improves cellular uptake properties due to its zwitterionic properties.

In some instances, the 5′-vinylphosphonate modified nucleotide is further modified at the 2′ hydroxyl group in a locked or bridged ribose modification (e.g., locked nucleic acid or LNA) in which the oxygen molecule bound at the 2′ carbon is linked to the 4′ carbon by a methylene group, thus forming a 2′-C,4′-C-oxy-methylene-linked bicyclic ribonucleotide monomer. Exemplary representations of the chemical structure of 5′-vinylphosphonate modified LNA are illustrated below, wherein J is an internucleotide linkage.

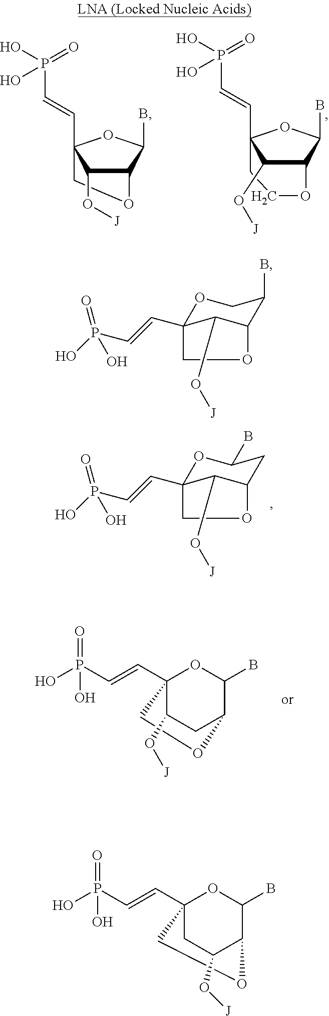

In some embodiments, additional modifications at the 2′ hydroxyl group include 2′-deoxy, T-deoxy-2′-fluoro, 2′-O-aminopropyl (2′-O-AP), 2′-O-dimethylaminoethyl (2′-O-DMAOE), 2′-O-dimethylaminopropyl (2′-O-DMAP), T-O-dimethylaminoethyloxyethyl (2′-O-DMAEOE), or 2′-O—N-methylacetamido (2′-O-NMA).

In some embodiments, a nucleotide analogue comprises a modified base such as, but not limited to, 5-propynyluridine, 5-propynylcytidine, 6-methyladenine, 6-methylguanine, N, N-dimethyladenine, 2-propyladenine, 2propylguanine, 2-aminoadenine, 1-methylinosine, 3-methyluridine, 5-methylcytidine, 5-methyluridine and other nucleotides having a modification at the 5 position, 5-(2-amino) propyl uridine, 5-halocytidine, 5-halouridine, 4-acetylcytidine, 1-methyladenosine, 2-methyladenosine, 3-methylcytidine, 6-methyluridine, 2-methylguanosine, 7-methylguanosine, 2, 2-dimethylguanosine, 5-methylaminoethyluridine, 5-methyloxyuridine, deazanucleotides (such as 7-deaza-adenosine, 6-azouridine, 6-azocytidine, or 6-azothymidine), 5-methyl-2-thiouridine, other thio bases (such as 2-thiouridine, 4-thiouridine, and 2-thiocytidine), dihydrouridine, pseudouridine, queuosine, archaeosine, naphthyl and substituted naphthyl groups, any O- and N-alkylated purines and pyrimidines (such as N6-methyladenosine, 5-methylcarbonylmethyluridine, uridine 5-oxyacetic acid, pyridine-4-one, or pyridine-2-one), phenyl and modified phenyl groups such as aminophenol or 2,4, 6-trimethoxy benzene, modified cytosines that act as G-clamp nucleotides, 8-substituted adenines and guanines, 5-substituted uracils and thymines, azapyrimidines, carboxyhydroxyalkyl nucleotides, carboxyalkylaminoalkyi nucleotides, and alkylcarbonylalkylated nucleotides. 5′-Vinylphosphonate modified nucleotides also include those nucleotides that are modified with respect to the sugar moiety, as well as 5′-vinylphosphonate modified nucleotides having sugars or analogs thereof that are not ribosyl. For example, the sugar moieties, in some cases are or are based on, mannoses, arabinoses, glucopyranoses, galactopyranoses, 4′-thioribose, and other sugars, heterocycles, or carbocycles. The term nucleotide also includes what are known in the art as universal bases. By way of example, universal bases include but are not limited to 3-nitropyrrole, 5-nitroindole, or nebularine.

In some embodiments, a 5′-vinylphosphonate modified nucleotide analogue further comprises a morpholino, a peptide nucleic acid (PNA), a methylphosphonate nucleotide, a thiolphosphonate nucleotide, a 2′-fluoro N3-P5′-phosphoramidite, or a 1′, 5′-anhydrohexitol nucleic acid (HNA). Morpholino or phosphorodiamidate morpholino oligo (PMO) comprises synthetic molecules whose structure mimics natural nucleic acid structure but deviates from the normal sugar and phosphate structures. In some instances, the five member ribose ring is substituted with a six member morpholino ring containing four carbons, one nitrogen, and one oxygen. In some cases, the ribose monomers are linked by a phosphordiamidate group instead of a phosphate group. In such cases, the backbone alterations remove all positive and negative charges making morpholinos neutral molecules capable of crossing cellular membranes without the aid of cellular delivery agents such as those used by charged oligonucleotides. A non-limiting example of a 5′-vinylphosphonate modified morpholino oligonucleotide is illustrated below.

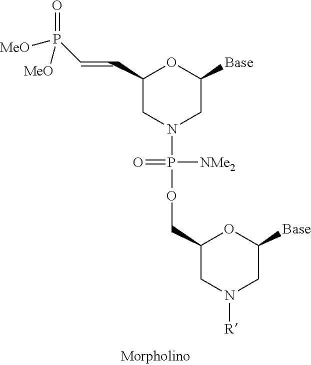

In some embodiments, a 5′-vinylphosphonate modified morpholino or PMO described above is a PMO comprising a positive or cationic charge. In some instances, the PMO is PMOplus (Sarepta). PMOplus refers to phosphorodiamidate morpholino oligomers comprising any number of (1-piperazino)phosphinylideneoxy, (1-(4-(omega-guanidino-alkanoyl))-piperazino)phosphinylideneoxy linkages (e.g., as such those described in PCT Publication No. WO2008/036127. In some cases, the PMO is a PMO described in U.S. Pat. No. 7,943,762.

In some embodiments, a morpholino or PMO described above is a PMO-X (Sarepta). In some cases, PMO-X refers to phosphorodiamidate morpholino oligomers comprising at least one linkage or at least one of the disclosed terminal modifications, such as those disclosed in PCT Publication No. WO2011/150408 and U.S. Publication No. 2012/0065169.

In some embodiments, a morpholino or PMO described above is a PMO as described in Table 5 of U.S. Publication No. 2014/0296321.

Exemplary representations of the chemical structure of 5′-vinylphosphonate modified nucleic acids are illustrated below, wherein J is an internucleotide linkage.

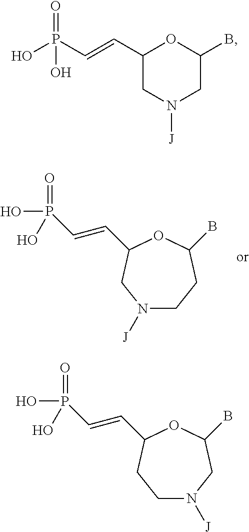

In some embodiments, peptide nucleic acid (PNA) does not contain sugar ring or phosphate linkage and the bases are attached and appropriately spaced by oligoglycine-like molecules, therefore, eliminating a backbone charge.

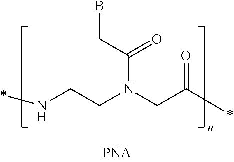

In some embodiments, one or more modifications of the 5′-vinylphosphonate modified oligonucleotide optionally occur at the internucleotide linkage. In some instances, modified internucleotide linkage includes, but is not limited to, phosphorothioates; phosphorodithioates; methylphosphonates; 5′-alkylenephosphonates; 5′-methylphosphonate; 3′-alkylene phosphonates; borontrifluoridates; borano phosphate esters and selenophosphates of 3′-5′linkage or 2′-5′linkage; phosphotriesters; thionoalkylphosphotriesters; hydrogen phosphonate linkages; alkyl phosphonates; alkylphosphonothioates; arylphosphonothioates; phosphoroselenoates; phosphorodiselenoates; phosphinates; phosphoramidates; 3′-alkylphosphoramidates; aminoalkylphosphoramidates; thionophosphoramidates; phosphoropiperazidates; phosphoroanilothioates; phosphoroanilidates; ketones; sulfones; sulfonamides; carbonates; carbamates; methylenehydrazos; methylenedimethylhydrazos; formacetals; thioformacetals; oximes; methyleneiminos; methylenemethyliminos; thioamidates; linkages with riboacetyl groups; aminoethyl glycine; silyl or siloxane linkages; alkyl or cycloalkyl linkages with or without heteroatoms of, for example, 1 to 10 carbons that are saturated or unsaturated and/or substituted and/or contain heteroatoms; linkages with morpholino structures, amides, or polyamides wherein the bases are attached to the aza nitrogens of the backbone directly or indirectly; and combinations thereof.

In some instances, the modification is a methyl or thiol modification such as methylphosphonate or thiolphosphonate modification. Exemplary thiolphosphonate nucleotide (left), phosphorodithioates (center) and methylphosphonate nucleotide (right) are illustrated below.

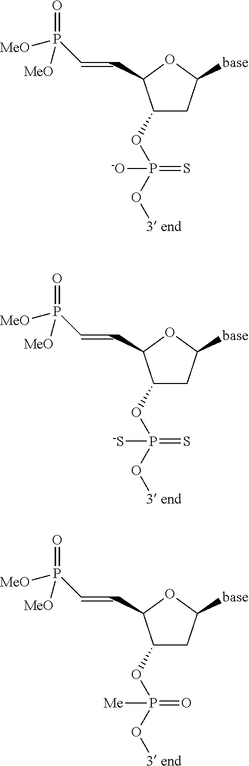

In some instances, a 5′-vinylphosphonate modified nucleotide includes, but is not limited to, phosphoramidites illustrated as:

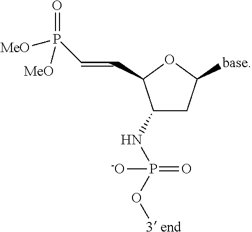

In some instances, the modified internucleotide linkage is a phosphorodiamidate linkage. A non-limiting example of a phosphorodiamidate linkage with a morpholino system is shown below.

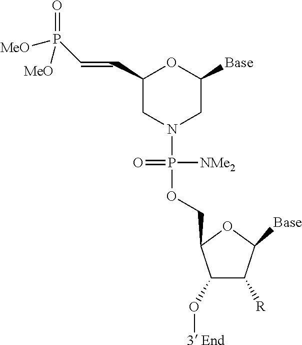

In some instances, the modified internucleotide linkage is a methylphosphonate linkage. A non-limiting example of a methylphosphonate linkage is shown below.

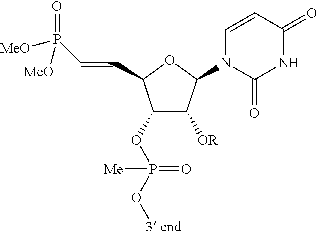

In some instances, the modified internucleotide linkage is a amide linkage. A non-limiting example of an amide linkage is shown below.

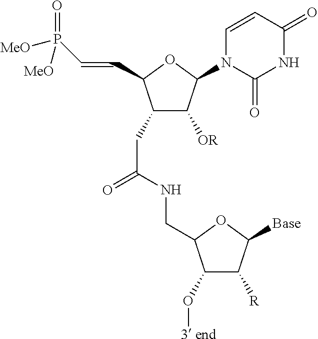

In some instances, a 5′-vinylphosphonate modified nucleotide includes, but is not limited to, the modified nucleic acid illustrated illustrated below.

In some embodiments, one or more modifications comprise a modified phosphate backbone in which the modification generates a neutral or uncharged backbone. In some instances, the phosphate backbone is modified by alkylation to generate an uncharged or neutral phosphate backbone. As used herein, alkylation includes methylation, ethylation, and propylation. In some cases, an alkyl group, as used herein in the context of alkylation, refers to a linear or branched saturated hydrocarbon group containing from 1 to 6 carbon atoms. In some instances, exemplary alkyl groups include, but are not limited to, methyl, ethyl, n-propyl, isopropyl, n-butyl, isobutyl, sec-butyl, tert-butyl, n-pentyl, isopentyl, neopentyl, hexyl, isohexyl, 1, 1-dimethylbutyl, 2,2-dimethylbutyl, 3.3-dimethylbutyl, and 2-ethylbutyl groups. In some cases, a modified phosphate is a phosphate group as described in U.S. Pat. No. 9,481,905.

In some embodiments, additional modified phosphate backbones comprise methylphosphonate, ethylphosphonate, methylthiophosphonate, or methoxyphosphonate. In some cases, the modified phosphate is methylphosphonate. In some cases, the modified phosphate is ethylphosphonate. In some cases, the modified phosphate is methylthiophosphonate. In some cases, the modified phosphate is methoxyphosphonate.

In some embodiments, one or more modifications further optionally include modifications of the ribose moiety, phosphate backbone and the nucleoside, or modifications of the nucleotide analogues at the 3′ or the 5′ terminus. For example, the 3′ terminus optionally include a 3′ cationic group, or by inverting the nucleoside at the 3-terminus with a 3′-3′ linkage. In another alternative, the 3′-terminus is optionally conjugated with an aminoalkyl group, e.g., a 3′ C5-aminoalkyl dT. In an additional alternative, the 3′-terminus is optionally conjugated with an abasic site, e.g., with an apurinic or apyrimidinic site.

In some embodiments, the polynucleic acid molecule comprises one or more of the artificial nucleotide analogues described herein. In some instances, the 5′-vinylphosphonate modified polynucleic acid molecule comprises 1, 2, 3, 4, 5, 6, 7, 8, 9, 10, 11, 12, 13, 14, 15, 16, 17, 18, 20, 25, or more of the artificial 5′-vinylphosphonate modified nucleotide analogues described herein. In some embodiments, the artificial 5′-vinylphosphonate modified nucleotide analogues include 2′-O-methyl, 2′-O-methoxyethyl (2′-O-MOE), 2′-O-aminopropyl, 2′-deoxy, T-deoxy-2′-fluoro, 2′-O-aminopropyl (2′-O-AP), 2′-O-dimethylaminoethyl (2′-O-DMAOE), 2′-O-dimethylaminopropyl (2′-O-DMAP), T-O-dimethylaminoethyloxyethyl (2′-O-DMAEOE), or 2′-O—N-methylacetamido (2′-O-NMA) modified, LNA, ENA, PNA, HNA, morpholino, methylphosphonate nucleotides, thiolphosphonate nucleotides, 2′-fluoro N3-P5′-phosphoramidites, or a combination thereof. In some instances, the 5′-vinylphosphonate modified polynucleic acid molecule comprises 1, 2, 3, 4, 5, 6, 7, 8, 9, 10, 11, 12, 13, 14, 15, 16, 17, 18, 20, 25, or more of the artificial nucleotide analogues selected from 2′-O-methyl, 2′-O-methoxyethyl (2′-O-MOE), 2′-O-aminopropyl, 2′-deoxy, T-deoxy-2′-fluoro, 2′-O-aminopropyl (2′-O-AP), 2′-O-dimethylaminoethyl (2′-O-DMAOE), 2′-O-dimethylaminopropyl (2′-O-DMAP), T-O-dimethylaminoethyloxyethyl (2′-O-DMAEOE), or 2′-O—N-methylacetamido (2′-O-NMA) modified, LNA, ENA, PNA, HNA, morpholino, methylphosphonate nucleotides, thiolphosphonate nucleotides, 2′-fluoro N3-P5′-phosphoramidites, or a combination thereof. In some instances, the 5′-vinylphosphonate modified polynucleic acid molecule comprises 1, 2, 3, 4, 5, 6, 7, 8, 9, 10, 11, 12, 13, 14, 15, 16, 17, 18, 20, 25, or more of 2′-O-methyl modified nucleotides. In some instances, the 5′-vinylphosphonate modified polynucleic acid molecule comprises 1, 2, 3, 4, 5, 6, 7, 8, 9, 10, 11, 12, 13, 14, 15, 16, 17, 18, 20, 25, or more of 2′-O-methoxyethyl (2′-O-MOE) modified nucleotides. In some instances, the 5′-vinylphosphonate modified polynucleic acid molecule comprises 1, 2, 3, 4, 5, 6, 7, 8, 9, 10, 11, 12, 13, 14, 15, 16, 17, 18, 20, 25, or more of thiolphosphonate nucleotides.

In some embodiments, the 5′-vinylphosphonate modified polynucleic acid molecule comprises at least about 1, about 2, about 3, about 4, about 5, about 6, about 7, about 8, about 9, about 10, about 11, about 12, about 13, about 14, about 15, about 16, about 17, about 18, about 19, about 20, about 21, about 22, or more modifications. In some instances, the polynucleic acid molecule is a polynucleic acid molecule of SEQ ID NOs: 16-45, 422-1173, 1195-1214, or 1215-1242

In some instances, the 5′-vinylphosphonate modified polynucleic acid molecule comprises at least about 1, about 2, about 3, about 4, about 5, about 6, about 7, about 8, about 9, about 10, about 11, about 12, about 13, about 14, about 15, about 16, about 17, about 18, about 19, about 20, about 21, about 22, or more modified nucleotides. In some instances, the polynucleic acid molecule is a polynucleic acid molecule of SEQ ID NOs: 16-45, 422-1173, 1195-1214, or 1215-1242.

In some instances, the 5′-vinylphosphonate modified polynucleic acid molecule comprises at least one of: from about 5% to about 100% modification, from about 10% to about 100% modification, from about 20% to about 100% modification, from about 30% to about 100% modification, from about 40% to about 100% modification, from about 50% to about 100% modification, from about 60% to about 100% modification, from about 70% to about 100% modification, from about 80% to about 100% modification, and from about 90% to about 100% modification. In some instances, the polynucleic acid molecule is a polynucleic acid molecule of SEQ ID NOs: 16-45, 422-1173, 1195-1214, or 1215-1242.

In some instances, about 5 to about 100% of the 5′-vinylphosphonate modified polynucleic acid molecule comprise the artificial nucleotide analogues described herein. In some instances, about 5%, 10%, 15%, 20%, 25%, 30%, 35%, 40%, 45%, 50%, 55%, 60%, 65%, 70%, 75%, 80%, 85%, 90%, 95%, or 100% of the polynucleic acid molecule comprise the artificial nucleotide analogues described herein. In some instances, about 5%, 10%, 15%, 20%, 25%, 30%, 35%, 40%, 45%, 50%, 55% 60%, 65%, 70%, 75%, 80%, 85%, 90%, 95%, or 100% of a polynucleic acid molecule of SEQ ID NOs: 16-45, 422-1173, 1195-1214, or 1215-1242 comprise the artificial nucleotide analogues described herein. In some instances, about 5%, 10%, 15%, 20%, 25%, 30%, 35%, 40%, 45%, 50%, 55%, 60%, 65%, 70%, 75%, 80%, 85%, 90%, 95%, or 100% of a polynucleic acid molecule of SEQ ID NOs: 16-45 comprise the artificial nucleotide analogues described herein. In some instances, about 5%, 10%, 15%, 20%, 25%, 30%, 35%, 40%, 45%, 50%, 55%, 60%, 65%, 70%, 75%, 80%, 85%, 90%, 95%, or 100% of a polynucleic acid molecule of SEQ ID NOs: 422-1173 comprise the artificial nucleotide analogues described herein. In some instances, about 5%, 10%, 15%, 20%, 25%, 30%, 35%, 40%, 45%, 50%, 55%, 60%, 65%, 70%, 75%, 80%, 85%, 90%, 95%, or 100% of a polynucleic acid molecule of SEQ ID NOs: 1195-1214 comprise the artificial nucleotide analogues described herein. In some instances, about 5%, 10%, 15%, 20%, 25%, 30%, 35%, 40%, 45%, 50%, 55%, 60%, 65%, 70%, 75%, 80%, 85%, 90%, 95%, or 100% of a polynucleic acid molecule of SEQ ID NOs: 1215-1242 comprise the artificial nucleotide analogues described herein. In some embodiments, the artificial nucleotide analogues include 2′-O-methyl, 2′-O-methoxyethyl (2′-O-MOE), 2′-O-aminopropyl, 2′-deoxy, T-deoxy-2′-fluoro, 2′-O-aminopropyl (2′-O-AP), 2′-O-dimethylaminoethyl (2′-O-DMAOE), 2′-O-dimethylaminopropyl (2′-O-DMAP), T-O-dimethylaminoethyloxyethyl (2′-O-DMAEOE), or 2′-O—N-methylacetamido (2′-O-NMA) modified, LNA, ENA, PNA, HNA, morpholino, methylphosphonate nucleotides, thiolphosphonate nucleotides, 2′-fluoro N3-P5′-phosphoramidites, or a combination thereof.

In some cases, one or more of the artificial 5′-vinylphosphonate modified nucleotide analogues described herein are resistant toward nucleases such as for example ribonuclease such as RNase H, deoxyribunuclease such as DNase, or exonuclease such as 5′-3′ exonuclease and 3′-5′ exonuclease when compared to natural polynucleic acid molecules. In some instances, artificial nucleotide analogues comprising 2′-O-methyl, 2′-O-methoxyethyl (2′-O-MOE), 2′-O-aminopropyl, 2′-deoxy, T-deoxy-2′-fluoro, 2′-O-aminopropyl (2′-O-AP), 2′-O-dimethylaminoethyl (2′-O-DMAOE), 2′-O-dimethylaminopropyl (2′-O-DMAP), T-O-dimethylaminoethyloxyethyl (2′-O-DMAEOE), or 2′-O—N-methylacetamido (2′-O-NMA) modified, LNA, ENA, PNA, HNA, morpholino, methylphosphonate nucleotides, thiolphosphonate nucleotides, 2′-fluoro N3-P5′-phosphoramidites, or combinations thereof are resistant toward nucleases such as for example ribonuclease such as RNase H, deoxyribunuclease such as DNase, or exonuclease such as 5′-3′ exonuclease and 3′-5′ exonuclease. In some instances, 2′-O-methyl modified polynucleic acid molecule is nuclease resistant (e.g., RNase H, DNase, 5′-3′ exonuclease or 3′-5′ exonuclease resistant). In some instances, 2′O-methoxyethyl (2′-O-MOE) modified polynucleic acid molecule is nuclease resistant (e.g., RNase H, DNase, 5′-3′ exonuclease or 3′-5′ exonuclease resistant). In some instances, 2′-O-aminopropyl modified polynucleic acid molecule is nuclease resistant (e.g., RNase H, DNase, 5′-3′ exonuclease or 3′-5′ exonuclease resistant). In some instances, 2′-deoxy modified polynucleic acid molecule is nuclease resistant (e.g., RNase H, DNase, 5′-3′ exonuclease or 3′-5′ exonuclease resistant). In some instances, T-deoxy-2′-fluoro modified polynucleic acid molecule is nuclease resistant (e.g., RNase H, DNase, 5′-3′ exonuclease or 3′-5′ exonuclease resistant). In some instances, 2′-O-aminopropyl (2′-O-AP) modified polynucleic acid molecule is nuclease resistant (e.g., RNase H, DNase, 5′-3′ exonuclease or 3′-5′ exonuclease resistant). In some instances, 2′-O-dimethylaminoethyl (2′-O-DMAOE) modified polynucleic acid molecule is nuclease resistant (e.g., RNase H, DNase, 5′-3′ exonuclease or 3′-5′ exonuclease resistant). In some instances, 2′-O-dimethylaminopropyl (2′-O-DMAP) modified polynucleic acid molecule is nuclease resistant (e.g., RNase H, DNase, 5′-3′ exonuclease or 3′-5′ exonuclease resistant). In some instances, T-O-dimethylaminoethyloxyethyl (2′-O-DMAEOE) modified polynucleic acid molecule is nuclease resistant (e.g., RNase H, DNase, 5′-3′ exonuclease or 3′-5′ exonuclease resistant). In some instances, 2′-O—N-methylacetamido (2′-O-NMA) modified polynucleic acid molecule is nuclease resistant (e.g., RNase H, DNase, 5′-3′ exonuclease or 3′-5′ exonuclease resistant). In some instances, LNA-modified polynucleic acid molecule is nuclease resistant (e.g., RNase H, DNase, 5′-3′ exonuclease or 3′-5′ exonuclease resistant). In some instances, ENA-modified polynucleic acid molecule is nuclease resistant (e.g., RNase H, DNase, 5′-3′ exonuclease or 3′-5′ exonuclease resistant). In some instances, HNA-modified polynucleic acid molecule is nuclease resistant (e.g., RNase H, DNase, 5′-3′ exonuclease or 3′-5′ exonuclease resistant). Morpholinos may be nuclease resistant (e.g., RNase H, DNase, 5′-3′ exonuclease or 3′-5′ exonuclease resistant). In some instances, PNA-modified polynucleic acid molecule is resistant to nucleases (e.g., RNase H, DNase, 5′-3′ exonuclease or 3′-5′ exonuclease resistant). In some instances, methylphosphonate nucleotide-modified polynucleic acid molecule is nuclease resistant (e.g., RNase H, DNase, 5′-3′ exonuclease or 3′-5′ exonuclease resistant). In some instances, thiolphosphonate nucleotide-modified polynucleic acid molecule is nuclease resistant (e.g., RNase H, DNase, 5′-3′ exonuclease or 3′-5′ exonuclease resistant). In some instances, polynucleic acid molecule comprising 2′-fluoro N3-P5′-phosphoramidites is nuclease resistant (e.g., RNase H, DNase, 5′-3′ exonuclease or 3′-5′ exonuclease resistant). In some instances, the 5′ conjugates described herein inhibit 5′-3′ exonucleolytic cleavage. In some instances, the 3′ conjugates described herein inhibit 3′-5′ exonucleolytic cleavage.

In some embodiments, one or more of the artificial 5′-vinylphosphonate modified nucleotide analogues described herein have increased binding affinity toward their mRNA target relative to an equivalent natural polynucleic acid molecule. The one or more of the artificial nucleotide analogues comprising 2′-O-methyl, 2′-O-methoxyethyl (2′-O-MOE), 2′-O-aminopropyl, 2′-deoxy, T-deoxy-2′-fluoro, 2′-O-aminopropyl (2′-O-AP), 2′-O-dimethylaminoethyl (2′-O-DMAOE), 2′-O-dimethylaminopropyl (2′-O-DMAP), T-O-dimethylaminoethyloxyethyl (2′-O-DMAEOE), or 2′-O—N-methylacetamido (2′-O-NMA) modified, LNA, ENA, PNA, HNA, morpholino, methylphosphonate nucleotides, thiolphosphonate nucleotides, or 2′-fluoro N3-P5′-phosphoramidites can have increased binding affinity toward their mRNA target relative to an equivalent natural polynucleic acid molecule. In some instances, 2′-O-methyl modified polynucleic acid molecule has increased binding affinity toward their mRNA target relative to an equivalent natural polynucleic acid molecule. In some instances, 2′-O-methoxyethyl (2′-O-MOE) modified polynucleic acid molecule has increased binding affinity toward their mRNA target relative to an equivalent natural polynucleic acid molecule. In some instances, 2′-O-aminopropyl modified polynucleic acid molecule has increased binding affinity toward their mRNA target relative to an equivalent natural polynucleic acid molecule. In some instances, 2′-deoxy modified polynucleic acid molecule has increased binding affinity toward their mRNA target relative to an equivalent natural polynucleic acid molecule. In some instances, T-deoxy-2′-fluoro modified polynucleic acid molecule has increased binding affinity toward their mRNA target relative to an equivalent natural polynucleic acid molecule. In some instances, 2′-O-aminopropyl (2′-O-AP) modified polynucleic acid molecule has increased binding affinity toward their mRNA target relative to an equivalent natural polynucleic acid molecule. In some instances, 2′-O-dimethylaminoethyl (2′-O-DMAOE) modified polynucleic acid molecule has increased binding affinity toward their mRNA target relative to an equivalent natural polynucleic acid molecule. In some instances, 2′-O-dimethylaminopropyl (2′-O-DMAP) modified polynucleic acid molecule has increased binding affinity toward their mRNA target relative to an equivalent natural polynucleic acid molecule. In some instances, T-O-dimethylaminoethyloxyethyl (2′-O-DMAEOE) modified polynucleic acid molecule has increased binding affinity toward their mRNA target relative to an equivalent natural polynucleic acid molecule. In some instances, 2′-O—N-methylacetamido (2′-O-NMA) modified polynucleic acid molecule has increased binding affinity toward their mRNA target relative to an equivalent natural polynucleic acid molecule. In some instances, LNA-modified polynucleic acid molecule has increased binding affinity toward their mRNA target relative to an equivalent natural polynucleic acid molecule. In some instances, ENA-modified polynucleic acid molecule has increased binding affinity toward their mRNA target relative to an equivalent natural polynucleic acid molecule. In some instances, PNA-modified polynucleic acid molecule has increased binding affinity toward their mRNA target relative to an equivalent natural polynucleic acid molecule. In some instances, HNA-modified polynucleic acid molecule has increased binding affinity toward their mRNA target relative to an equivalent natural polynucleic acid molecule. In some instances, morpholino-modified polynucleic acid molecule has increased binding affinity toward their mRNA target relative to an equivalent natural polynucleic acid molecule. In some instances, methylphosphonate nucleotide-modified polynucleic acid molecule has increased binding affinity toward their mRNA target relative to an equivalent natural polynucleic acid molecule. In some instances, thiolphosphonate nucleotide-modified polynucleic acid molecule has increased binding affinity toward their mRNA target relative to an equivalent natural polynucleic acid molecule. In some instances, polynucleic acid molecule comprising 2′-fluoro N3-P5′-phosphoramidites has increased binding affinity toward their mRNA target relative to an equivalent natural polynucleic acid molecule. In some cases, the increased affinity is illustrated with a lower Kd, a higher melt temperature (Tm), or a combination thereof.

In some embodiments, a 5′-vinylphosphonate modified polynucleic acid molecule described herein is a chirally pure (or stereo pure) polynucleic acid molecule, or a polynucleic acid molecule comprising a single enantiomer. In some instances, the polynucleic acid molecule comprises L-nucleotide. In some instances, the polynucleic acid molecule comprises D-nucleotides. In some instance, a polynucleic acid molecule composition comprises less than 30%, 25%, 20%, 15%, 10%, 5%, 4%, 3%, 2%, 1%, or less of its mirror enantiomer. In some cases, a polynucleic acid molecule composition comprises less than 30%, 25%, 20%, 15%, 10%, 5%, 4%, 3%, 2%, 1%, or less of a racemic mixture. In some instances, the polynucleic acid molecule is a polynucleic acid molecule described in: U.S. Patent Publication Nos: 2014/194610 and 2015/211006; and PCT Publication No.: WO2015107425.

In some embodiments, a polynucleic acid molecule described herein is further modified to include an aptamer-conjugating moiety. In some instances, the aptamer conjugating moiety is a DNA aptamer-conjugating moiety. In some instances, the aptamer-conjugating moiety is Alphamer (Centauri Therapeutics), which comprises an aptamer portion that recognizes a specific cell-surface target and a portion that presents a specific epitopes for attaching to circulating antibodies. In some instance, a polynucleic acid molecule described herein is further modified to include an aptamer-conjugating moiety as described in: U.S. Pat. Nos. 8,604,184, 8,591,910, and 7,850,975.

In additional embodiments, a polynucleic acid molecule described herein is modified to increase its stability. In some embodiment, the polynucleic acid molecule is RNA (e.g., siRNA), the polynucleic acid molecule is modified to increase its stability. In some instances, the polynucleic acid molecule is modified by one or more of the modifications described above to increase its stability. In some cases, the polynucleic acid molecule is modified at the 2′ hydroxyl position, such as by 2′-O-methyl, 2′-O-methoxyethyl (2′-O-MOE), 2′-O-aminopropyl, 2′-deoxy, T-deoxy-2′-fluoro, 2′-O-aminopropyl (2′-O-AP), 2′-O-dimethylaminoethyl (2′-O-DMAOE), 2′-O-dimethylaminopropyl (2′-O-DMAP), T-O-dimethylaminoethyloxyethyl (2′-O-DMAEOE), or 2′-O—N-methylacetamido (2′-O-NMA) modification or by a locked or bridged ribose conformation (e.g., LNA or ENA). In some cases, the polynucleic acid molecule is modified by 2′-O-methyl and/or 2′-O-methoxyethyl ribose. In some cases, the polynucleic acid molecule also includes morpholinos, PNAs, HNA, methylphosphonate nucleotides, thiolphosphonate nucleotides, and/or 2′-fluoro N3-P5′-phosphoramidites to increase its stability. In some instances, the polynucleic acid molecule is a chirally pure (or stereo pure) polynucleic acid molecule. In some instances, the chirally pure (or stereo pure) polynucleic acid molecule is modified to increase its stability. Suitable modifications to the RNA to increase stability for delivery will be apparent to the skilled person.

In some embodiments, a polynucleic acid molecule described herein has RNAi activity that modulates expression of RNA encoded by a gene described supra. In some instances, a polynucleic acid molecule described herein is a double-stranded siRNA molecule that down-regulates expression of a gene, wherein one of the strands of the double-stranded siRNA molecule comprises a nucleotide sequence that is complementary to a nucleotide sequence of the gene or RNA encoded by the gene or a portion thereof, and wherein the second strand of the double-stranded siRNA molecule comprises a nucleotide sequence substantially similar to the nucleotide sequence of the gene or RNA encoded by the gene or a portion thereof. In some cases, a polynucleic acid molecule described herein is a double-stranded siRNA molecule that down-regulates expression of a gene, wherein each strand of the siRNA molecule comprises about 15 to 25, 18 to 24, or 19 to about 23 nucleotides, and wherein each strand comprises at least about 14, 17, or 19 nucleotides that are complementary to the nucleotides of the other strand. In some cases, a polynucleic acid molecule described herein is a double-stranded siRNA molecule that down-regulates expression of a gene, wherein each strand of the siRNA molecule comprises about 19 to about 23 nucleotides, and wherein each strand comprises at least about 19 nucleotides that are complementary to the nucleotides of the other strand. In some instances, the gene is KRAS, EGFR, AR, HPRT1, CNNTB1 (β-catenin), or β-catenin associated genes.

In some embodiments, a polynucleic acid molecule described herein is constructed using chemical synthesis and/or enzymatic ligation reactions using procedures known in the art. For example, a polynucleic acid molecule is chemically synthesized using naturally occurring nucleotides or variously modified nucleotides designed to increase the biological stability of the molecules or to increase the physical stability of the duplex forned between the polynucleic acid molecule and target nucleic acids. Exemplary methods include those described in: U.S. Pat. Nos. 5,142,047; 5,185,444; 5,889,136; 6,008,400; and 6,111,086; PCT Publication No. WO2009099942; or European Publication No. 1579015. Additional exemplary methods include those described in: Griffey et al., “2′-O-aminiopropyl ribonucleotides: a zwitterionic modification that enhances the exonuclease resistance and biological activity of antisense oligonucleotides,” J. Med. Chem. 39(26):5100-5109 (1997)); Obika, et al. “Synthesis of 2′-O,4′-C-methyleneuridine and -cytidine. Novel bicyclic nucleosides having a fixed C3, -endo sugar puckering”. Tetrahedron Letters 38 (50): 8735 (1997); Koizumi, M. “ENA oligonucleotides as therapeutics”. Current opinion in molecular therapeutics 8 (2): 144-149 (2006); and Abramova et al., “Novel oligonucleotide analogues based on morpholino nucleoside subunits-antisense technologies: new chemical possibilities,” Indian Journal of Chemistry 48B:1721-1726 (2009). Alternatively, the polynucleic acid molecule is produced biologically using an expression vector into which a polynucleic acid molecule has been subeloned in an antisense orientation (i.e., RNA transcribed from the inserted polynucleic acid molecule will be of an antisense orientation to a target polynucleic acid molecule of interest).

One embodiment provides a molecule of Formula (I):

A-X—B—Y—C Formula I

wherein,
A is a binding moiety;
B is a polynucleotide;
C is a polymer;
X is a bond or a first non-polymeric linker; and
Y is a bond or a second linker;
wherein the polynucleotide comprises at least one 5′-vinylphosphonate modified non-natural nucleotide; and
wherein A and C are not attached to B at the same terminus.

Another embodiment provides the molecule of Formula (I), wherein the polynucleotide further comprises, at least one modified internucleotide linkage, or at least one inverted abasic moiety;

Another embodiment provides the molecule of Formula (1), wherein the at least one 5′-vinylphosphonate modified non-natural nucleotide is located at the 5′-terminus of the polynucleotide.

Another embodiment provides the molecule of Formula (I), wherein the at least one 5′-vinylphosphonate modified non-natural nucleotide is located at an internucleotide linkage of the polynucleotide.

Another embodiment provides the molecule of Formula (1), wherein the at least one 5′-vinylphosphonate modified non-natural nucleotide is further modified at the 2′-position.

Another embodiment provides the molecule of Formula (1), wherein the 2′-modification is selected from 2′-O-methyl, 2′-O-methoxyethyl (2′-O-MOE), 2′-deoxy, T-deoxy-2′-fluoro, 2′-O-arnminopropyl (2′-OAP), 2′-O-dimethylaminoethyl (2′-O-DMAOE), 2′-O-dimethylarninopropyl (2′-O-DMAP), T-O-dimethylaminoethyloxyethyl (2′-O-DMAEOE), or 2′-O—N-methylacetarido (2′-C-NMA) modified nucleotide.

Another embodiment provides the molecule of Formula (I), wherein the at least one 5′-vinylphosphonate modified non-natural nucleotide is selected from:

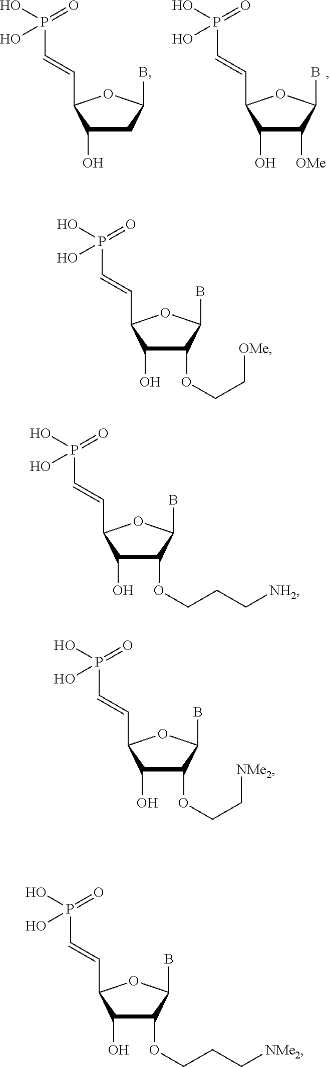

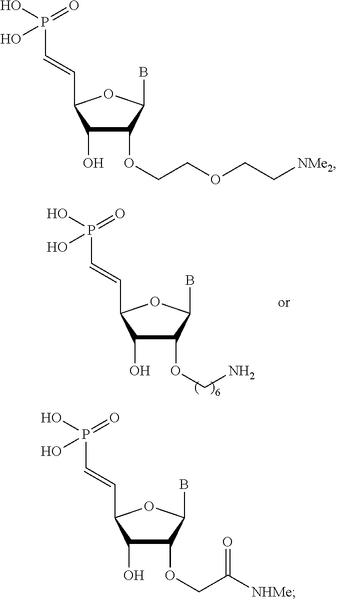

wherein B is a heterocyclic base moiety.

Another embodiment provides the molecule of Formula (1), wherein the at least one 5′-vinylphosphonate modified non-natural nucleotide is selected from:

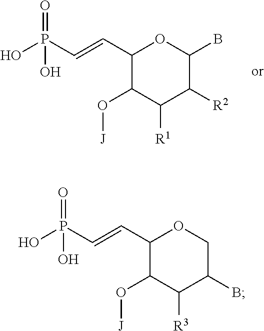

wherein is a heterocyclic base moiety;
R1, R2, and R3 are independently selected from hydrogen, halogen, alkyl or alkoxy; and
J is an internucleotide linking group linking to the adjacent nucleotide of the polynucleotide.

Another embodiment provides the molecule of Formula (T), wherein the at least one 5′-vinylphosphonate modified non-natural nucleotide is selected from:

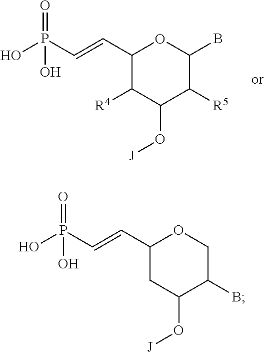

wherein B is a heterocyclic base moiety;
R4, and R5 are independently selected from hydrogen, halogen, alkyl or alkoxy; and
J is an internucleotide linking group linking to the adjacent nucleotide of the polynucleotide.

Another embodiment provides the molecule of Formula (1), wherein the at least one 5′-vinylphosphonate modified non-natural nucleotide is selected from:

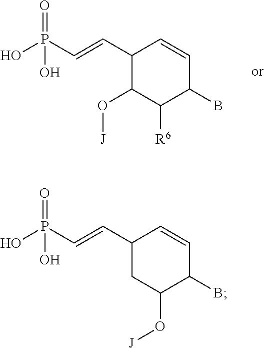

wherein B is a heterocyclic base moiety;
R6 is selected from hydrogen, halogen, alkyl or alkoxy; and
J is an internucleotide linking group linking to the adjacent nucleotide of the polynucleotide.

Another embodiment provides the molecule of Formula (I), wherein the at least one 5′-vinylphosphonate modified non-natural nucleotide is selected from locked nucleic acid (LNA) or ethylene nucleic acid (ENA).

Another embodiment provides the molecule of Formula (1), wherein the at least one 5′-vinyliphosphonate modified non-natural nucleotide is selected from:

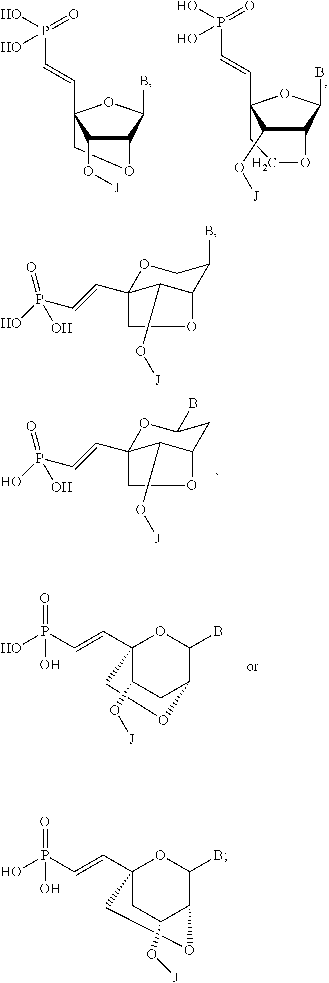

wherein B is a heterocyclic base moiety; and
J is an internucleotide linking group linking to the adjacent nucleotide of the polynucleotide.

Another embodiment provides the molecule of Formula (1), wherein the at least one 5′-vinylphosphonate modified non-natural nucleotide is selected from:

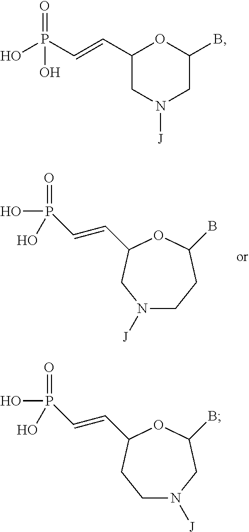

wherein B is a heterocyclic base moiety; and
J is an internucleotide linking group linking to the adjacent nucleotide of the polynucleotide.

Another embodiment provides the molecule of Formula (1), wherein the at least one 5′-vinylphosphonate modified non-natural nucleotide is:

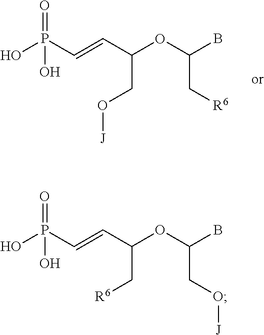

wherein B is a heterocyclic base moiety;
R6 is selected from hydrogen, halogen, alkyl or alkoxy; and
J is an internucleotide linking group linking to the adjacent nucleotide of the polynucleotide.

Another embodiment provides the molecule of Formula (I), wherein the at least one modified internucleotide linkage comprises a phosphorothioate linkage, phosphorodithioate linkage, a phosphorodiamidate linkage, a methylphosphonate linkage, or an amide linkage.

Another embodiment provides the molecule of Formula (1), wherein the at least one inverted abasic moiety is at least one terminus.

One embodiment provides an oligonucleotide of Formula (II), wherein the oligonucleotide comprises at least one 5′-vinylphosphonate modified non-natural nucleotide.

Another embodiment provides the oligonucleotide of Formula (II), wherein the oligonucleotide further comprises, at least one modified internucleotide linkage, or at least one inverted abasic moiety;

Another embodiment provides the oligonucleotide of Formula (II), wherein the at least one 5′-vinylphosphonate modified non-natural nucleotide is located at the 5′-terminus of the polynucleotide.

Another embodiment provides the oligonucleotide of Formula (II), wherein the at least one 5′-vinylphosphonate modified non-natural nucleotide is located at an internucleotide linkage of the polynucleotide.

Another embodiment provides the oligonucleotide of Formula (II), wherein the at least one 5′-vinylphosphonate modified non-natural nucleotide is further modified at the 2′-position. Another embodiment provides the oligonucleotide of Formula (II), wherein the 2′-modification is selected from 2′-O-methyl, 2′-O-methoxyethyl (2′-O-MOE), 2′-deoxy, T-deoxy-2′-fluoro, 2-O-aminopropyl (2′-O-AP), 2′-O-dimethylaminoethyl (2′-O-DMAOE), 2′-O-dimethylaminopropyl (2′-O-DMAP), T-O-dimethylaminoethyloxyethyl (2′-O-DMAEOE), or 2′-O—N-methylacetamido (2′-O-NMA) modified nucleotide.

Another embodiment provides the oligonucleotide of Formula (II), wherein the at least one 5′-vinylphosphonate modified non-natural nucleotide is selected from:

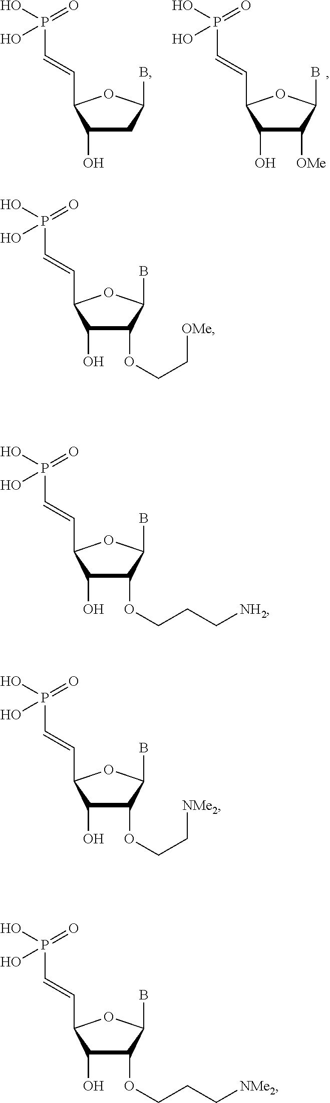

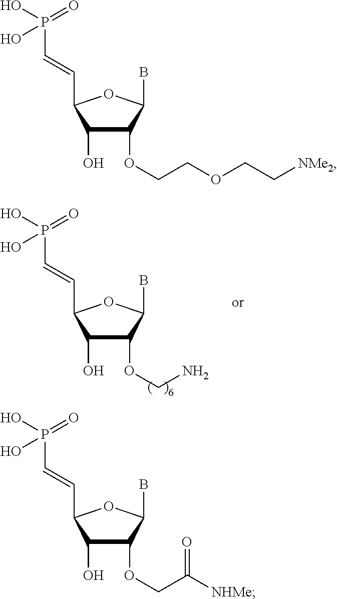

wherein B is a heterocyclic base moiety.

Another embodiment provides the oligonucleotide of Formula (II), wherein the at least one 5′-vinylphosphonate modified non-natural nucleotide is selected from:

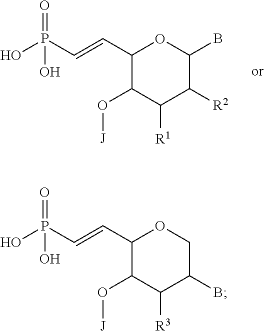

wherein B is a heterocyclic base moiety
R1, R2, and R3 are independently selected from hydrogen, halogen, alkyl or alkoxy; and
J is an internucleotide linking group linking to the adjacent nucleotide of the polynucleotide.

Another embodiment provides the oligonucleotide of Formula (II), wherein the at least one 5′-vinylphosphonate modified non-natural nucleotide is selected from:

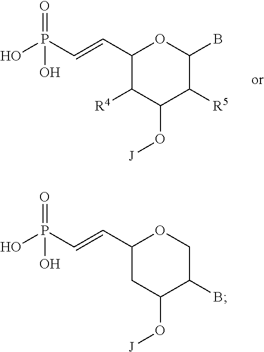

wherein B is a heterocyclic base moiety:
R4, and R5 are independently selected from hydrogen, halogen, alkyl or alkoxy; and
J is an internucleotide linking group linking to the adjacent nucleotide of the polynucleotide.

Another embodiment provides the oligonucleotide of Formula (II), wherein the at least one 5′-vinylphosphonate modified non-natural nucleotide is selected from:

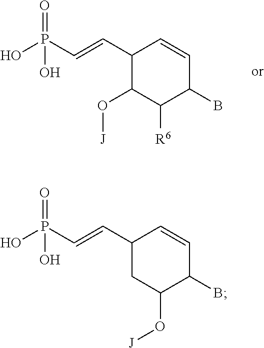

wherein B is a heterocyclic base moiety:
R6 is selected from hydrogen, halogen, alkyl or alkoxy; and
J is an internucleotide linking group linking to the adjacent nucleotide of the polynucleotide.

Another embodiment provides the oligonucleotide of Formula (II), wherein the at least one 5′-vinylphosphonate modified non-natural nucleotide is selected from locked nucleic acid (LNA) or ethylene nucleic acid (ENA).

Another embodiment provides the oligonucleotide of Formula (II), wherein the at least one 5′-vinylphosphonate modified non-natural nucleotide is selected from:

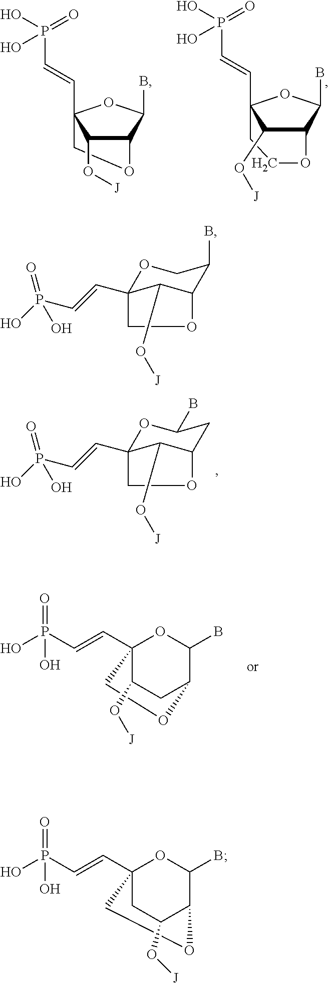

wherein B is a heterocyclic base moiety; and
J is an internucleotide linking group linking to the adjacent nucleotide of the polynucleotide.

Another embodiment provides the oligonucleotide of Formula (II), wherein the at least one 5′-vinylphosphonate modified non-natural nucleotide is selected from:

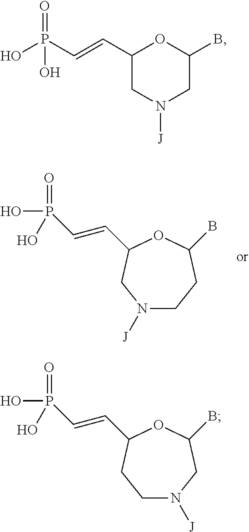

wherein B is a heterocyclic base moiety; and
J is an internucleotide linking group linking to the adjacent nucleotide of the polynucleotide.

Another embodiment provides the oligonucleotide of Formula (II), wherein the at least one 5′ vinylphosphonate modified non-natural nucleotide is:

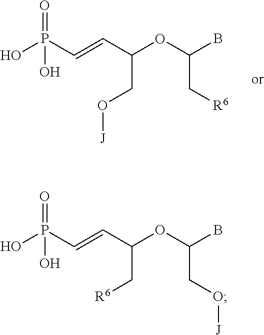

wherein B is a heterocyclic base moiety;
R6 is selected from hydrogen, halogen, alkyl or alkoxy; and
J is an internucleotide linking group linking to the adjacent nucleotide of the polynucleotide.

Another embodiment provides the oligonucleotide of Formula (II), wherein the at least one modified internucleotide linkage comprises a phosphorothioate linkage, phosphorodithioate linkage, a phosphorodiamidate linkage, a methylphosphonate linkage, or an amide linkage.

Another embodiment provides the oligonucleotide of Formula (II), wherein the at least one inverted abasic moiety is at least one terminus.

Another embodiment provides the oligonucleotide of Formula (II), wherein the oligonucleotide is single stranded. Another embodiment provides the oligonucleotide of Formula (II), wherein the oligonucleotide is double stranded stranded.

Another embodiment provides the oligonucleotide of Formula (II), wherein the oligonucleotide is from 2 to about 100 residues in length. Another embodiment provides the oligonucleotide of Formula (II), wherein the oligonucleotide is from 2 to about 90 residues in length. Another embodiment provides the oligonucleotide of Formula (II), wherein the oligonucleotide is from 2 to about 80 residues in length Another embodiment provides the oligonucleotide of Formula (II), wherein the oligonucleotide is from 2 to about 70 residues in length. Another embodiment provides the oligonucleotide of Formula (II), wherein the oligonucleotide is from 2 to about 60 residues in length. Another embodiment provides the oligonucleotide of Formula (II), wherein the oligonucleotide is from 2 to about 50 residues in length. Another embodiment provides the oligonucleotide of Formula (II), wherein the oligonucleotide is from 2 to about 40 residues in length. Another embodiment provides the oligonucleotide of Formula (II), wherein the oligonucleotide is from 2 to about 30 residues in length. Another embodiment provides the oligonucleotide of Formula (II), wherein the oligonucleotide is from 2 to about 20 residues in length. Another embodiment provides the oligonucleotide of Formula (II), wherein the oligonucleotide is from 2 to about 10 residues in length. Another embodiment provides the oligonucleotide of Formula (II), wherein the oligonucleotide is from 8 to about 30 residues in length. Another embodiment provides the oligonucleotide of Formula (II), wherein the oligonucleotide is from 10 to about 30 residues in length. Another embodiment provides the oligonucleotide of Formula (II), wherein the oligonucleotide is from 14 to about 30 residues in length, Another embodiment provides the oligonucleotide of Formula (II), wherein the oligonucleotide is from 18 to about 30 residues in length. Another embodiment provides the oligonucleotide of Formula (II), wherein the oligonucleotide is from 22 to about 30 residues in length. Another embodiment provides the oligonucleotide of Formula (II), wherein the oligonucleotide is from 26 to about 30 residues in length.

One embodiment provides a compound suitable for the synthesis of oligonucleotides selected from the group:

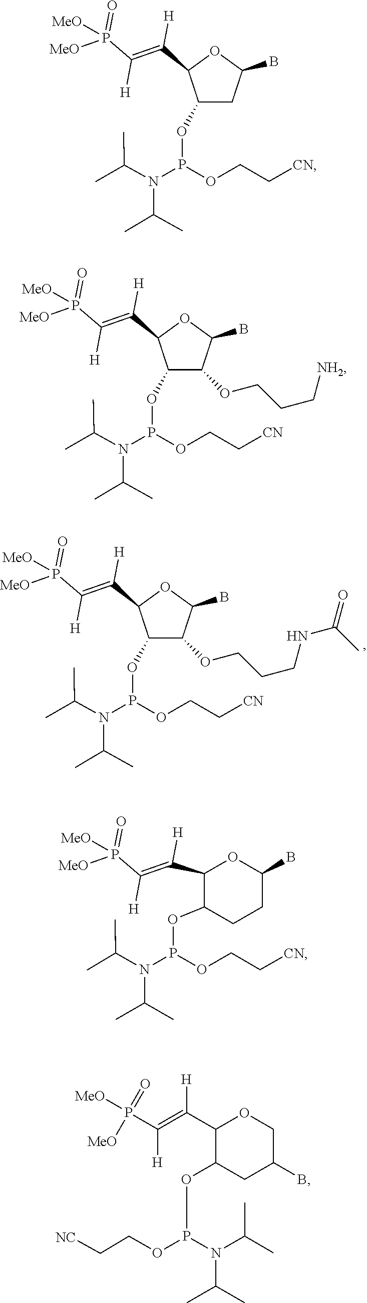

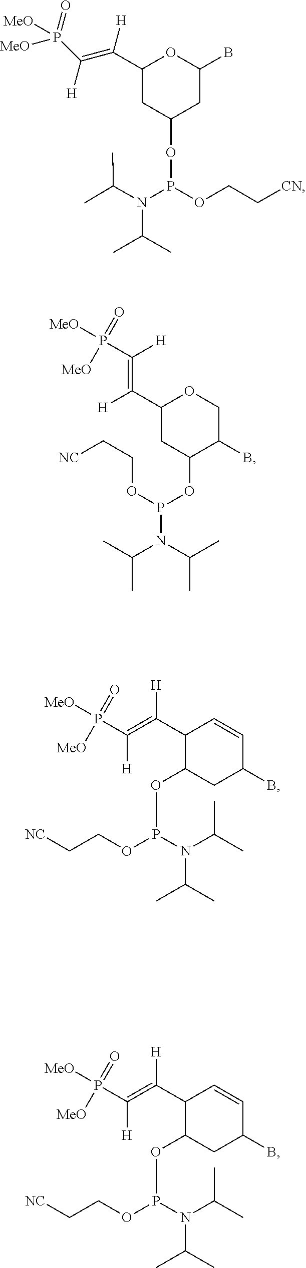

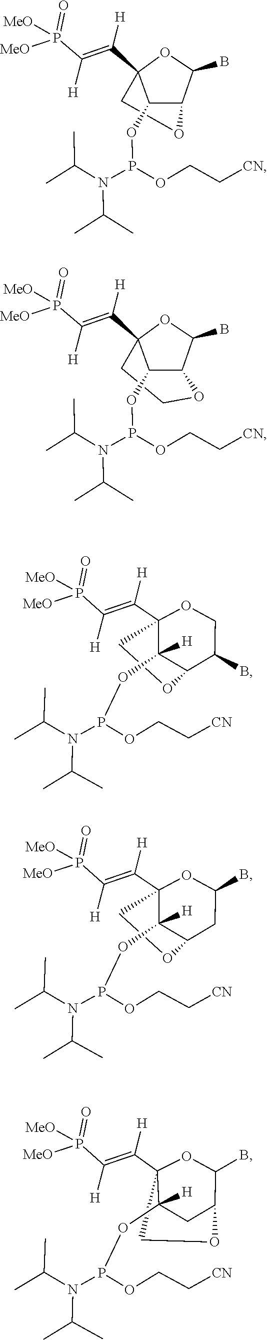

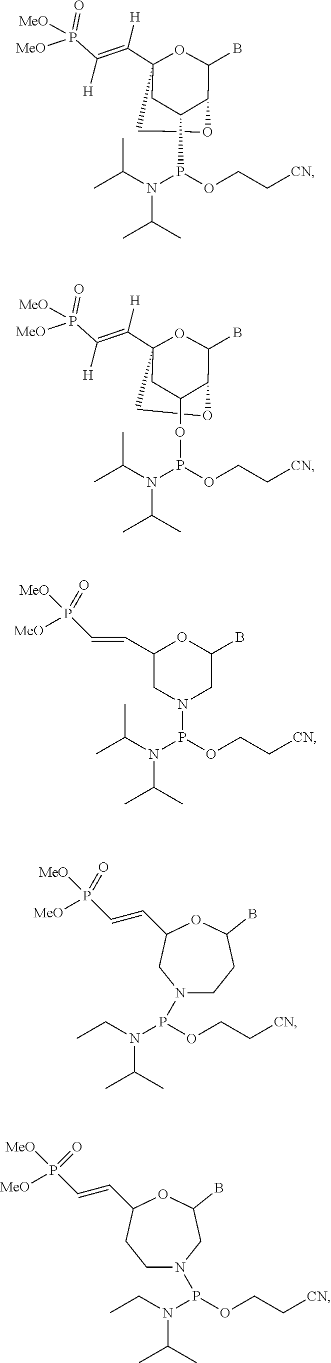

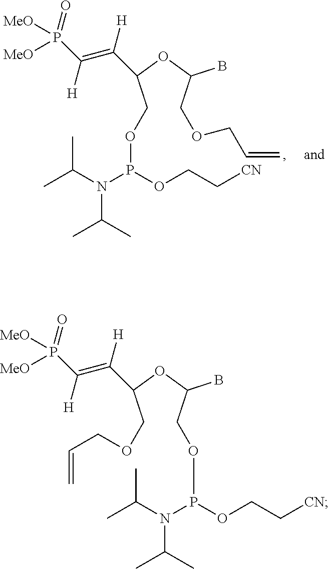

wherein B is a heterocyclic base moiety.

### Conjugation Chemistry

In some embodiments, a polynucleic acid molecule is conjugated to a binding moiety. In some instances, the binding moiety comprises amino acids, peptides, polypeptides, proteins, antibodies, antigens, toxins, hormones, lipids, nucleotides, nucleosides, sugars, carbohydrates, polymers such as polyethylene glycol and polypropylene glycol, as well as analogs or derivatives of all of these classes of substances. Additional examples of binding moiety also include steroids, such as cholesterol, phospholipids, di- and triacylglycerols, fatty acids, hydrocarbons (e.g., saturated, unsaturated, or contains substitutions), enzyme substrates, biotin, digoxigenin, and polysaccharides. In some instances, the binding moiety is an antibody or binding fragment thereof. In some instances, the polynucleic acid molecule is further conjugated to a polymer, and optionally an endosomolytic moiety.

In some embodiments, the polynucleic acid molecule is conjugated to the binding moiety by a chemical ligation process. In some instances, the polynucleic acid molecule is conjugated to the binding moiety by a native ligation. In some instances, the conjugation is as described in: Dawson, et al. “Synthesis of proteins by native chemical ligation,” Science 1994, 266, 776-779; Dawson, et al. “Modulation of Reactivity in Native Chemical Ligation through the Use of Thiol Additives,” J. Am. Chem. Soc. 1997, 119, 4325-4329; Hackeng, et al. “Protein synthesis by native chemical ligation: Expanded scope by using straightforward methodology,” Proc. Natl. Acad. Sci. USA 1999, 96, 10068-10073; or Wu, et al. “Building complex glycopeptides: Development of a cysteine-free native chemical ligation protocol,” Angew. Chem. Int. Ed. 2006, 45, 4116-4125. In some instances, the conjugation is as described in U.S. Pat. No. 8,936,910. In some embodiments, the polynucleic acid molecule is conjugated to the binding moiety either site-specifically or non-specifically via native ligation chemistry.

In some instances, the polynucleic acid molecule is conjugated to the binding moiety by a site-directed method utilizing a “traceless” coupling technology (Philochem). In some instances, the “traceless” coupling technology utilizes an N-terminal 1,2-aminothiol group on the binding moiety which is then conjugate with a polynucleic acid molecule containing an aldehyde group. (see Casi et al., “Site-specific traceless coupling of potent cytotoxic drugs to recombinant antibodies for pharmacodelivery,” JACS 134(13): 5887-5892 (2012))

In some instances, the polynucleic acid molecule is conjugated to the binding moiety by a site-directed method utilizing an unnatural amino acid incorporated into the binding moiety. In some instances, the unnatural amino acid comprises p-acetylphenylalanine (pAcPhe). In some instances, the keto group of pAcPhe is selectively coupled to an alkoxy-amine derivatived conjugating moiety to form an oxime bond. (see Axup et al., “Synthesis of site-specific antibody-drug conjugates using unnatural amino acids,” PNAS 109(40): 16101-16106 (2012)).

In some instances, the polynucleic acid molecule is conjugated to the binding moiety by a site-directed method utilizing an enzyme-catalyzed process. In some instances, the site-directed method utilizes SMARTag™ technology (Redwood). In some instances, the SMARTag™ technology comprises generation of a formylglycine (FGly) residue from cysteine by formylglycine-generating enzyme (FGE) through an oxidation process under the presence of an aldehyde tag and the subsequent conjugation of FGly to an alkylhydraine-functionalized polynucleic acid molecule via hydrazino-Pictet-Spengler (HIPS) ligation. (see Wu et al., “Site-specific chemical modification of recombinant proteins produced in mammalian cells by using the genetically encoded aldehyde tag,” wPNAS 106(9): 3000-3005 (2009); Agarwal, et al., “A Pictet-Spengler ligation for protein chemical modification,” PNAS 110(1): 46-51 (2013))

In some instances, the enzyme-catalyzed process comprises microbial transglutaminase (mTG). In some cases, the polynucleic acid molecule is conjugated to the binding moiety utilizing a microbial transglutaminze catalyzed process. In some instances, mTG catalyzes the formation of a covalent bond between the amide side chain of a glutamine within the recognition sequence and a primary amine of a functionalized polynucleic acid molecule. In some instances, mTG is produced from Streptomyces mobarensis. (see Strop et al., “Location matters: site of conjugation modulates stability and pharmacokinetics of antibody drug conjugates,” Chemistry and Biology 20(2) 161-167 (2013))

In some instances, the polynucleic acid molecule is conjugated to the binding moiety by a method as described in PCT Publication No. WO2014/140317, which utilizes a sequence-specific transpeptidase.

In some instances, the polynucleic acid molecule is conjugated to the binding moiety by a method as described in U.S. Patent Publication Nos. 2015/0105539 and 2015/0105540.

### Binding Moiety

In some embodiments, the binding moiety A is a polypeptide. In some instances, the polypeptide is an antibody or its fragment thereof. In some cases, the fragment is a binding fragment. In some instances, the antibody or binding fragment thereof comprises a humanized antibody or binding fragment thereof, murine antibody or binding fragment thereof, chimeric antibody or binding fragment thereof, monoclonal antibody or binding fragment thereof, monovalent Fab′, divalent Fab₂, F(ab)₃ fragments, single-chain variable fragment (scFv), bis-scFv, (scFv)₂, diabody, minibody, nanobody, triabody, tetrabody, disulfide stabilized Fv protein (dsFv), single-domain antibody (sdAb), Ig NAR, camelid antibody or binding fragment thereof, bispecific antibody or biding fragment thereof, or a chemically modified derivative thereof.

In some instances, A is an antibody or binding fragment thereof. In some instances, A is a humanized antibody or binding fragment thereof, murine antibody or binding fragment thereof, chimeric antibody or binding fragment thereof, monoclonal antibody or binding fragment thereof, monovalent Fab′, divalent Fab₂, F(ab)′₃ fragments, single-chain variable fragment (scFv), bis-scFv, (scFv)₂, diabody, minibody, nanobody, triabody, tetrabody, disulfide stabilized Fv protein (“dsFv”), single-domain antibody (sdAb), Ig NAR, camelid antibody or binding fragment thereof, bispecific antibody or biding fragment thereof, or a chemically modified derivative thereof. In some instances, A is a humanized antibody or binding fragment thereof. In some instances, A is a murine antibody or binding fragment thereof. In some instances, A is a chimeric antibody or binding fragment thereof. In some instances, A is a monoclonal antibody or binding fragment thereof. In some instances, A is a monovalent Fab′. In some instances, A is a diavalent Fab₂. In some instances, A is a single-chain variable fragment (scFv).

In some embodiments, the binding moiety A is a bispecific antibody or binding fragment thereof. In some instances, the bispecific antibody is a trifunctional antibody or a bispecific mini-antibody. In some cases, the bispecific antibody is a trifunctional antibody. In some instances, the trifunctional antibody is a full length monoclonal antibody comprising binding sites for two different antigens. Exemplary trifunctional antibodies include catumaxomab (which targets EpCAM and CD3; Fresenius Biotech/Trion Pharma), ertumaxomab (targets HER2/neu/CD3; Fresenius Biotech/Trion Pharma), lymphomun FBTA05 (targets CD20/CD3; Fresenius Biotech/Trion Pharma), RG7221 (R05520985; targets Angiopoietin 2/VEGF; Roche), RG7597 (targets Herl/Her3; Genentech/Roche), MM141 (targets IGF1R/Her3; Merrimack), ABT122 (targets TNFα/IL17; Abbvie), ABT981 (targets IL1α/IL1β; Abbott), LY3164530 (targets Herl/cMET; Eli Lilly), and TRBS07 (Ektomab; targets GD2/CD3; Trion Research Gmbh). Additional exemplary trifunctional antibodies include mAb² from F-star Biotechnology Ltd. In some instances, A is a bispecific trifunctional antibody. In some embodiments, A is a bispecific trifunctional antibody selected from: catumaxomab (which targets EpCAM and CD3; Fresenius Biotech/Trion Pharma), ertumaxomab (targets HER2/neu/CD3; Fresenius Biotech/Trion Pharma), lymphomun FBTA05 (targets CD20/CD3; Fresenius Biotech/Trion Pharma), RG7221 (R05520985; targets Angiopoietin 2/VEGF; Roche), RG7597 (targets Her1/Her3; Genentech/Roche), MM141 (targets IGF1R/Her3; Merrimack), ABT122 (targets TNFα/IL17; Abbvie), ABT981 (targets IL1α/IL1β; Abbott), LY3164530 (targets Her1/cMET; Eli Lilly), TRBS07 (Ektomab; targets GD2/CD3; Trion Research Gmbh), and a mAb² from F-star Biotechnology Ltd.

In some cases, the bispecific antibody is a bispecific mini-antibody. In some instances, the bispecific mini-antibody comprises divalent Fab₂, F(ab)′₃ fragments, bis-scFv, (scFv)₂, diabody, minibody, triabody, tetrabody or a bi-specific T-cell engager (BiTE). In some embodiments, the bi-specific T-cell engager is a fusion protein that contains two single-chain variable fragments (scFvs) in which the two scFvs target epitopes of two different antigens. Exemplary bispecific mini-antibodies include, but are not limited to, DART (dual-affinity re-targeting platform; MacroGenics), blinatumomab (MT103 or AMG103; which targets CD19/CD3; Micromet), MT111 (targets CEA/CD3; Micromet/Amegen), MT112 (BAY2010112; targets PSMA/CD3; Micromet/Bayer), MT110 (AMG 110; targets EPCAM/CD3; Amgen/Micromet), MGD006 (targets CD123/CD3; MacroGenics), MGD007 (targets GPA33/CD3; MacroGenics), BI1034020 (targets two different epitopes on β-amyloid; Ablynx), ALX0761 (targets IL17A/IL17F; Ablynx), TF2 (targets CEA/hepten; Immunomedics), IL-17/IL-34 biAb (BMS), AFM13 (targets CD30/CD16; Affimed), AFM11 (targets CD19/CD3; Affimed), and domain antibodies (dAbs from Domantis/GSK).

In some embodiments, the binding moiety A is a bispecific mini-antibody. In some instances, A is a bispecific Fab₂. In some instances, A is a bispecific F(ab)′₃ fragment. In some cases, A is a bispecific bis-scFv. In some cases, A is a bispecific (scFv)₂. In some embodiments, A is a bispecific diabody. In some embodiments, A is a bispecific minibody. In some embodiments, A is a bispecific triabody. In other embodiments, A is a bispecific tetrabody. In other embodiments, A is a bi-specific T-cell engager (BiTE). In additional embodiments, A is a bispecific mini-antibody selected from: DART (dual-affinity re-targeting platform; MacroGenics), blinatumomab (MT103 or AMG103; which targets CD19/CD3; Micromet), MT111 (targets CEA/CD3; Micromet/Amegen), MT112 (BAY2010112; targets PSMA/CD3; Micromet/Bayer), MT 110 (AMG 110; targets EPCAM/CD3; Amgen/Micromet), MGD006 (targets CD123/CD3; MacroGenics), MGD007 (targets GPA33/CD3; MacroGenics), BI1034020 (targets two different epitopes on β-amyloid; Ablynx), ALX0761 (targets IL17A/IL17F; Ablynx), TF2 (targets CEA/hepten; Immunomedics), IL-17/IL-34 biAb (BMS), AFM13 (targets CD30/CD16; Affimed), AFM11 (targets CD19/CD3; Affimed), and domain antibodies (dAbs from Domantis/GSK).

In some embodiments, the binding moiety A is a trispecific antibody. In some instances, the trispecific antibody comprises F(ab)′₃ fragments or a triabody. In some instances, A is a trispecific F(ab)′₃ fragment. In some cases, A is a triabody. In some embodiments, A is a trispecific antibody as described in Dimas, et al., “Development of a trispecific antibody designed to simultaneously and efficiently target three different antigens on tumor cells,” Mol. Pharmaceutics, 12(9): 3490-3501 (2015).

In some embodiments, the binding moiety A is an antibody or binding fragment thereof that recognizes a cell surface protein. In some instances, the cell surface protein is an antigen expressed by a cancerous cell. Exemplary cancer antigens include, but are not limited to, alpha fetoprotein, ASLG659, B7-H3, BAFF-R, Brevican, CA125 (MUC16), CA15-3, CA19-9, carcinoembryonic antigen (CEA), CA242, CRIPTO (CR, CR1, CRGF, CRIPTO, TDGF1, teratocarcinoma-derived growth factor), CTLA-4, CXCR5, E16 (LAT1, SLC7A5), FcRH2 (IFGP4, IRTA4, SPAP1A (SH2 domain containing phosphatase anchor protein 1a), SPAP1B, SPAP1C), epidermal growth factor, ETBR, Fc receptor-like protein 1 (FCRH1), GEDA, HLA-DOB (Beta subunit of MHC class II molecule (Ia antigen), human chorionic gonadotropin, ICOS, IL-2 receptor, IL20Rα, Immunoglobulin superfamily receptor translocation associated 2 (IRTA2), L6, Lewis Y, Lewis X, MAGE-1, MAGE-2, MAGE-3, MAGE 4, MART1, mesothelin, MDP, MPF (SMR, MSLN), MCP1 (CCL2), macrophage inhibitory factor (MIF), MPG, MSG783, mucin, MUC1-KLH, Napi3b (SLC34A2), nectin-4, Neu oncogene product, NCA, placental alkaline phosphatase, prostate specific membrane antigen (PMSA), prostatic acid phosphatase, PSCA hlg, p97, Purinergic receptor P2X ligand-gated ion channel 5 (P2X5), LY64 (Lymphocyte antigen 64 (RP105), gp100, P21, six transmembrane epithelial antigen of prostate (STEAP1), STEAP2, Sema 5b, tumor-associated glycoprotein 72 (TAG-72), TrpM4 (BR22450, FLJ20041, TRPM4, TRPM4B, transient receptor potential cation channel, subfamily M, member 4) and the like.

In some instances, the cell surface protein comprises clusters of differentiation (CD) cell surface markers. Exemplary CD cell surface markers include, but are not limited to, CD1, CD2, CD3, CD4, CD5, CD6, CD7, CD8, CD9, CD10, CD11a, CD11b, CD11c, CD11d, CDw12, CD13, CD14, CD15, CD15s, CD16, CDw17, CD18, CD19, CD20, CD21, CD22, CD23, CD24, CD25, CD26, CD27, CD28, CD29, CD30, CD31, CD32, CD33, CD34, CD35, CD36, CD37, CD38, CD39, CD40, CD41, CD42, CD43, CD44, CD45, CD45RO, CD45RA, CD45RB, CD46, CD47, CD48, CD49a, CD49b, CD49c, CD49d, CD49e, CD49f, CD50, CD51, CD52, CD53, CD54, CD55, CD56, CD57, CD58, CD59, CDw60, CD61, CD62E, CD62L (L-selectin), CD62P, CD63, CD64, CD65, CD66a, CD66b, CD66c, CD66d, CD66e, CD79 (e.g., CD79a, CD79b), CD90, CD95 (Fas), CD103, CD104, CD125 (IL5RA), CD134 (OX40), CD137 (4-1BB), CD152 (CTLA-4), CD221, CD274, CD279 (PD-1), CD319 (SLAMF7), CD326 (EpCAM), and the like.

In some instances, the binding moiety A is an antibody or binding fragment thereof that recognizes a cancer antigen. In some instances, the binding moiety A is an antibody or binding fragment thereof that recognizes alpha fetoprotein, ASLG659, B7-H3, BAFF-R, Brevican, CA125 (MUC16), CA15-3, CA19-9, carcinoembryonic antigen (CEA), CA242, CRIPTO (CR, CR1, CRGF, CRIPTO, TDGF1, teratocarcinoma-derived growth factor), CTLA-4, CXCR5, E16 (LAT1, SLC7A5), FcRH2 (IFGP4, IRTA4, SPAP1A (SH2 domain containing phosphatase anchor protein 1a), SPAP1B, SPAP1C), epidermal growth factor, ETBR, Fc receptor-like protein 1 (FCRH1), GEDA, HLA-DOB (Beta subunit of MHC class II molecule (Ia antigen), human chorionic gonadotropin, ICOS, IL-2 receptor, IL20Rα, Immunoglobulin superfamily receptor translocation associated 2 (IRTA2), L6, Lewis Y, Lewis X, MAGE-1, MAGE-2, MAGE-3, MAGE 4, MART1, mesothelin, MCP1 (CCL2), MDP, macrophage inhibitory factor (MIF), MPF (SMR, MSLN), MPG, MSG783, mucin, MUC1-KLH, Napi3b (SLC34A2), nectin-4, Neu oncogene product, NCA, placental alkaline phosphatase, prostate specific membrane antigen (PMSA), prostatic acid phosphatase, PSCA hlg, p97, Purinergic receptor P2X ligand-gated ion channel 5 (P2X5), LY64 (Lymphocyte antigen 64 (RP105), gp100, P21, six transmembrane epithelial antigen of prostate (STEAP1), STEAP2, Sema 5b, tumor-associated glycoprotein 72 (TAG-72), TrpM4 (BR22450, FLJ20041, TRPM4, TRPM4B, transient receptor potential cation channel, subfamily M, member 4) or a combination thereof.

In some instances, the binding moiety A is an antibody or binding fragment thereof that recognizes a CD cell surface marker. In some instances, the binding moiety A is an antibody or binding fragment thereof that recognizes CD1, CD2, CD3, CD4, CD5, CD6, CD7, CD8, CD9, CD10, CD11a, CD11b, CD11c, CD11d, CDw12, CD13, CD14, CD15, CD15s, CD16, CDw17, CD18, CD19, CD20, CD21, CD22, CD23, CD24, CD25, CD26, CD27, CD28, CD29, CD30, CD31, CD32, CD33, CD34, CD35, CD36, CD37, CD38, CD39, CD40, CD41, CD42, CD43, CD44, CD45, CD45RO, CD45RA, CD45RB, CD46, CD47, CD48, CD49a, CD49b, CD49c, CD49d, CD49e, CD49f, CD50, CD51, CD52, CD53, CD54, CD55, CD56, CD57, CD58, CD59, CDw60, CD61, CD62E, CD62L (L-selectin), CD62P, CD63, CD64, CD65, CD66a, CD66b, CD66c, CD66d, CD66e, CD79 (e.g., CD79a, CD79b), CD90, CD95 (Fas), CD103, CD104, CD125 (IL5RA), CD134 (OX40), CD137 (4-1BB), CD152 (CTLA-4), CD221, CD274, CD279 (PD-1), CD319 (SLAMF7), CD326 (EpCAM), or a combination thereof.

In some embodiments, the antibody or binding fragment thereof comprises zalutumumab (HuMax-EFGr, Genmab), abagovomab (Menarini), abituzumab (Merck), adecatumumab (MT201), alacizumab pegol, alemtuzumab (Campath®, MabCampath, or Campath-1H; Leukosite), AlloMune (BioTransplant), amatuximab (Morphotek, Inc.), anti-VEGF (Genetech), anatumomab mafenatox, apolizumab (hulD10), ascrinvacumab (Pfizer Inc.), atezolizumab (MPDL3280A; Genentech/Roche), B43.13 (OvaRex, AltaRex Corporation), basiliximab (Simulect®, Novartis), belimumab (Benlysta®, GlaxoSmithKline), bevacizumab (Avastin®, Genentech), blinatumomab (Blincyto, AMG103; Amgen), BEC2 (ImGlone Systems Inc.), carlumab (Janssen Biotech), catumaxomab (Removab, Trion Pharma), CEAcide (Immunomedics), Cetuximab (Erbitux®, ImClone), citatuzumab bogatox (VB6-845), cixutumumab (IMC-A12, ImClone Systems Inc.), conatumumab (AMG 655, Amgen), dacetuzumab (SGN-40, huS2C6; Seattle Genetics, Inc.), daratumumab (Darzalex®, Janssen Biotech), detumomab, drozitumab (Genentech), durvalumab (MedImmune), dusigitumab (MedImmune), edrecolomab (MAb17-1A, Panorex, Glaxo Wellcome), elotuzumab (Empliciti™, Bristol-Myers Squibb), emibetuzumab (Eli Lilly), enavatuzumab (Facet Biotech Corp.), enfortumab vedotin (Seattle Genetics, Inc.), enoblituzumab (MGA271, MacroGenics, Inc.), ensituxumab (Neogenix Oncology, Inc.), epratuzumab (LymphoCide, Immunomedics, Inc.), ertumaxomab (Rexomun®, Trion Pharma), etaracizumab (Abegrin, MedImmune), farletuzumab (MORAb-003, Morphotek, Inc), FBTA05 (Lymphomun, Trion Pharma), ficlatuzumab (AVEO Pharmaceuticals), figitumumab (CP-751871, Pfizer), flanvotumab (ImClone Systems), fresolimumab (GC1008, Aanofi-Aventis), futuximab, glaximab, ganitumab (Amgen), girentuximab (Rencarex®, Wilex AG), IMAB362 (Claudiximab, Ganymed Pharmaceuticals AG), imalumab (Baxalta), IMC-1C1 1 (ImClone Systems), IMC-C225 (Imclone Systems Inc.), imgatuzumab (Genentech/Roche), intetumumab (Centocor, Inc.), ipilimumab (Yervoy®, Bristol-Myers Squibb), iratumumab (Medarex, Inc.), isatuximab (SAR650984, Sanofi-Aventis), labetuzumab (CEA-CIDE, Immunomedics), lexatumumab (ETR2-ST01, Cambridge Antibody Technology), lintuzumab (SGN-33, Seattle Genetics), lucatumumab (Novartis), lumiliximab, mapatumumab (HGS-ETR1, Human Genome Sciences), matuzumab (EMD 72000, Merck), milatuzumab (hLL1, Immunomedics, Inc.), mitumomab (BEC-2, ImClone Systems), narnatumab (ImClone Systems), necitumumab (Portrazza™, Eli Lilly), nesvacumab (Regeneron Pharmaceuticals), nimotuzumab (h-R3, BIOMAb EGFR, TheraCIM, Theraloc, or CIMAher; Biotech Pharmaceutical Co.), nivolumab (Opdivo®, Bristol-Myers Squibb), obinutuzumab (Gazyva or Gazyvaro; Hoffmann-La Roche), ocaratuzumab (AME-133v, LY2469298; Mentrik Biotech, LLC), ofatumumab (Arzerra®, Genmab), onartuzumab (Genentech), Ontuxizumab (Morphotek, Inc.), oregovomab (OvaRex®, AltaRex Corp.), otlertuzumab (Emergent BioSolutions), panitumumab (ABX-EGF, Amgen), pankomab (Glycotope GMBH), parsatuzumab (Genentech), patritumab, pembrolizumab (Keytruda®, Merck), pemtumomab (Theragyn, Antisoma), pertuzumab (Perjeta, Genentech), pidilizumab (CT-011, Medivation), polatuzumab vedotin (Genentech/Roche), pritumumab, racotumomab (Vaxira®, Recombio), ramucirumab (Cyramza®, ImClone Systems Inc.), rituximab (Rituxan®, Genentech), robatumumab (Schering-Plough), Seribantumab (Sanofi/Merrimack Pharmaceuticals, Inc.), sibrotuzumab, siltuximab (Sylvant™, Janssen Biotech), Smart M195 (Protein Design Labs, Inc.), Smart ID10 (Protein Design Labs, Inc.), tabalumab (LY2127399, Eli Lilly), taplitumomab paptox, tenatumomab, teprotumumab (Roche), tetulomab, TGN1412 (CD28-SuperMAB or TAB08), tigatuzumab (CD-1008, Daiichi Sankyo), tositumomab, trastuzumab (Herceptin®), tremelimumab (CP-672,206; Pfizer), tucotuzumab celmoleukin (EMD Pharmaceuticals), ublituximab, urelumab (BMS-663513, Bristol-Myers Squibb), volociximab (M200, Biogen Idec), zatuximab, and the like.

In some embodiments, the binding moiety A comprises zalutumumab (HuMax-EFGr, Genmab), abagovomab (Menarini), abituzumab (Merck), adecatumumab (MT201), alacizumab pegol, alemtuzumab (Campath®, MabCampath, or Campath-1H; Leukosite), AlloMune (BioTransplant), amatuximab (Morphotek, Inc.), anti-VEGF (Genetech), anatumomab mafenatox, apolizumab (hulD10), ascrinvacumab (Pfizer Inc.), atezolizumab (MPDL3280A; Genentech/Roche), B43.13 (OvaRex, AltaRex Corporation), basiliximab (Simulect®, Novartis), belimumab (Benlysta®, GlaxoSmithKline), bevacizumab (Avastin®, Genentech), blinatumomab (Blincyto, AMG103; Amgen), BEC2 (ImGlone Systems Inc.), carlumab (Janssen Biotech), catumaxomab (Removab, Trion Pharma), CEAcide (Immunomedics), Cetuximab (Erbitux®, ImClone), citatuzumab bogatox (VB6-845), cixutumumab (IMC-A12, ImClone Systems Inc.), conatumumab (AMG 655, Amgen), dacetuzumab (SGN-40, huS2C6; Seattle Genetics, Inc.), daratumumab (Darzalex®, Janssen Biotech), detumomab, drozitumab (Genentech), durvalumab (MedImmune), dusigitumab (MedImmune), edrecolomab (MAbl7-1A, Panorex, Glaxo Wellcome), elotuzumab (Empliciti™, Bristol-Myers Squibb), emibetuzumab (Eli Lilly), enavatuzumab (Facet Biotech Corp.), enfortumab vedotin (Seattle Genetics, Inc.), enoblituzumab (MGA271, MacroGenics, Inc.), ensituxumab (Neogenix Oncology, Inc.), epratuzumab (LymphoCide, Immunomedics, Inc.), ertumaxomab (Rexomun®, Trion Pharma), etaracizumab (Abegrin, MedImmune), farletuzumab (MORAb-003, Morphotek, Inc), FBTA05 (Lymphomun, Trion Pharma), ficlatuzumab (AVEO Pharmaceuticals), figitumumab (CP-751871, Pfizer), flanvotumab (ImClone Systems), fresolimumab (GC1008, Aanofi-Aventis), futuximab, glaximab, ganitumab (Amgen), girentuximab (Rencarex®, Wilex AG), IMAB362 (Claudiximab, Ganymed Pharmaceuticals AG), imalumab (Baxalta), IMC-1C1 1 (ImClone Systems), IMC-C225 (Imclone Systems Inc.), imgatuzumab (Genentech/Roche), intetumumab (Centocor, Inc.), ipilimumab (Yervoy®, Bristol-Myers Squibb), iratumumab (Medarex, Inc.), isatuximab (SAR650984, Sanofi-Aventis), labetuzumab (CEA-CIDE, Immunomedics), lexatumumab (ETR2-ST01, Cambridge Antibody Technology), lintuzumab (SGN-33, Seattle Genetics), lucatumumab (Novartis), lumiliximab, mapatumumab (HGS-ETR1, Human Genome Sciences), matuzumab (EMD 72000, Merck), milatuzumab (hLL1, Immunomedics, Inc.), mitumomab (BEC-2, ImClone Systems), narnatumab (ImClone Systems), necitumumab (Portrazza™, Eli Lilly), nesvacumab (Regeneron Pharmaceuticals), nimotuzumab (h-R3, BIOMAb EGFR, TheraCIM, Theraloc, or CIMAher; Biotech Pharmaceutical Co.), nivolumab (Opdivo®, Bristol-Myers Squibb), obinutuzumab (Gazyva or Gazyvaro; Hoffmann-La Roche), ocaratuzumab (AME-133v, LY2469298; Mentrik Biotech, LLC), ofatumumab (Arzerra®, Genmab), onartuzumab (Genentech), Ontuxizumab (Morphotek, Inc.), oregovomab (OvaRex®, AltaRex Corp.), otlertuzumab (Emergent BioSolutions), panitumumab (ABX-EGF, Amgen), pankomab (Glycotope GMBH), parsatuzumab (Genentech), patritumab, pembrolizumab (Keytruda®, Merck), pemtumomab (Theragyn, Antisoma), pertuzumab (Perjeta, Genentech), pidilizumab (CT-011, Medivation), polatuzumab vedotin (Genentech/Roche), pritumumab, racotumomab (Vaxira®, Recombio), ramucirumab (Cyramza®, ImClone Systems Inc.), rituximab (Rituxan®, Genentech), robatumumab (Schering-Plough), Seribantumab (Sanofi/Merrimack Pharmaceuticals, Inc.), sibrotuzumab, siltuximab (Sylvant™, Janssen Biotech), Smart M195 (Protein Design Labs, Inc.), Smart ID10 (Protein Design Labs, Inc.), tabalumab (LY2127399, Eli Lilly), taplitumomab paptox, tenatumomab, teprotumumab (Roche), tetulomab, TGN1412 (CD28-SuperMAB or TAB08), tigatuzumab (CD-1008, Daiichi Sankyo), tositumomab, trastuzumab (Herceptin®), tremelimumab (CP-672,206; Pfizer), tucotuzumab celmoleukin (EMD Pharmaceuticals), ublituximab, urelumab (BMS-663513, Bristol-Myers Squibb), volociximab (M200, Biogen Idec), or zatuximab. In some embodiments, the binding moiety A is zalutumumab (HuMax-EFGr, by Genmab).

In some embodiments, the binding moiety A is conjugated according to Formula (I) to a polynucleic acid molecule (B), and a polymer (C), and optionally an endosomolytic moiety (D) according to Formula (II) described herein. In some instances, the polynucleic acid molecule comprises a sequence having at least 50%, 55%, 60%, 65%, 70%, 75%, 80%, 85%, 90%, 95%, 96%, 97%, 98%, 99%, or 100% sequence identity to a sequence listed in Tables 2, 4, 8, or 9. In some embodiments, the polynucleic acid molecule comprises a sequence having at least 50%, 55%, 60%, 65%, 70%, 75%, 80%, 85%, 90%, 95%, 96%, 97%, 98%, 99%, or 100% sequence identity to SEQ ID NOs: 16-45, 422-1173, 1195-1214, or 1215-1242. In some instances, the polynucleic acid molecule comprises a sequence selected from SEQ ID NOs: 16-45, 422-1173, 1195-1214, or 1215-1242. In some instances, the polymer C comprises polyalkylen oxide (e.g., polyethylene glycol). In some embodiments, the endosomolytic moiety D comprises INF7 or melittin, or their respective derivatives.

In some embodiments, the binding moiety A is conjugated to a polynucleic acid molecule (B), and a polymer (C), and optionally an endosomolytic moiety (D). In some instances, the binding moiety A is an antibody or binding fragment thereof.

In some embodiments, the binding moiety A is conjugated to a polynucleic acid molecule (B) non-specifically. In some instances, the binding moiety A is conjugated to a polynucleic acid molecule (B) via a lysine residue or a cysteine residue, in a non-site specific manner. In some instances, the binding moiety A is conjugated to a polynucleic acid molecule (B) via a lysine residue in a non-site specific manner. In some cases, the binding moiety A is conjugated to a polynucleic acid molecule (B) via a cysteine residue in a non-site specific manner. In some instances, the binding moiety A is an antibody or binding fragment thereof.

In some embodiments, the binding moiety A is conjugated to a polynucleic acid molecule (B) in a site-specific manner. In some instances, the binding moiety A is conjugated to a polynucleic acid molecule (B) through a lysine residue, a cysteine residue, at the 5′-terminus, at the 3′-terminus, an unnatural amino acid, or an enzyme-modified or enzyme-catalyzed residue, via a site-specific manner. In some instances, the binding moiety A is conjugated to a polynucleic acid molecule (B) through a lysine residue via a site-specific manner. In some instances, the binding moiety A is conjugated to a polynucleic acid molecule (B) through a cysteine residue via a site-specific manner. In some instances, the binding moiety A is conjugated to a polynucleic acid molecule (B) at the 5′-terminus via a site-specific manner. In some instances, the binding moiety A is conjugated to a polynucleic acid molecule (B) at the 3′-terminus via a site-specific manner. In some instances, the binding moiety A is conjugated to a polynucleic acid molecule (B) through an unnatural amino acid via a site-specific manner. In some instances, the binding moiety A is conjugated to a polynucleic acid molecule (B) through an enzyme-modified or enzyme-catalyzed residue via a site-specific manner. In some instances, the binding moiety A is an antibody or binding fragment thereof.

In some embodiments, one or more regions of a binding moiety A (e.g., an antibody or binding fragment thereof) is conjugated to a polynucleic acid molecule (B). In some instances, the one or more regions of a binding moiety A comprise the N-terminus, the C-terminus, in the constant region, at the hinge region, or the Fc region of the binding moiety A. In some instances, the polynucleic acid molecule (B) is conjugated to the N-terminus of the binding moiety A (e.g., the N-terminus of an antibody or binding fragment thereof). In some instances, the polynucleic acid molecule (B) is conjugated to the C-terminus of the binding moiety A (e.g., the N-terminus of an antibody or binding fragment thereof). In some instances, the polynucleic acid molecule (B) is conjugated to the constant region of the binding moiety A (e.g., the constant region of an antibody or binding fragment thereof). In some instances, the polynucleic acid molecule (B) is conjugated to the hinge region of the binding moiety A (e.g., the constant region of an antibody or binding fragment thereof). In some instances, the polynucleic acid molecule (B) is conjugated to the Fc region of the binding moiety A (e.g., the constant region of an antibody or binding fragment thereof).

In some embodiments, one or more polynucleic acid molecule (B) is conjugated to a binding moiety A. In some instances, about 1, 2, 3, 4, 5, 6, 7, 8, 9, 10, 11, 12, 13, 14, 15, 16, or more polynucleic acid molecules are conjugated to one binding moiety A. In some instances, about 1 polynucleic acid molecule is conjugated to one binding moiety A. In some instances, about 2 polynucleic acid molecules are conjugated to one binding moiety A. In some instances, about 3 polynucleic acid molecules are conjugated to one binding moiety A. In some instances, about 4 polynucleic acid molecules are conjugated to one binding moiety A. In some instances, about 5 polynucleic acid molecules are conjugated to one binding moiety A. In some instances, about 6 polynucleic acid molecules are conjugated to one binding moiety A. In some instances, about 7 polynucleic acid molecules are conjugated to one binding moiety A. In some instances, about 8 polynucleic acid molecules are conjugated to one binding moiety A. In some instances, about 9 polynucleic acid molecules are conjugated to one binding moiety A. In some instances, about 10 polynucleic acid molecules are conjugated to one binding moiety A. In some instances, about 11 polynucleic acid molecules are conjugated to one binding moiety A. In some instances, about 12 polynucleic acid molecules are conjugated to one binding moiety A. In some instances, about 13 polynucleic acid molecules are conjugated to one binding moiety A. In some instances, about 14 polynucleic acid molecules are conjugated to one binding moiety A. In some instances, about 15 polynucleic acid molecules are conjugated to one binding moiety A. In some instances, about 16 polynucleic acid molecules are conjugated to one binding moiety A. In some cases, the one or more polynucleic acid molecules are the same. In other cases, the one or more polynucleic acid molecules are different. In some instances, the binding moiety A is an antibody or binding fragment thereof.

In some embodiments, the number of polynucleic acid molecule (B) conjugated to a binding moiety A (e.g., an antibody or binding fragment thereof) forms a ratio. In some instances, the ratio is referred to as a DAR (drug-to-antibody) ratio, in which the drug as referred to herein is the polynucleic acid molecule (B). In some instances, the DAR ratio of the polynucleic acid molecule (B) to binding moiety A is about 1, 2, 3, 4, 5, 6, 7, 8, 9, 10, 11, 12, 13, 14, 15, 16, or greater. In some instances, the DAR ratio of the polynucleic acid molecule (B) to binding moiety A is about 1 or greater. In some instances, the DAR ratio of the polynucleic acid molecule (B) to binding moiety A is about 2 or greater. In some instances, the DAR ratio of the polynucleic acid molecule (B) to binding moiety A is about 3 or greater. In some instances, the DAR ratio of the polynucleic acid molecule (B) to binding moiety A is about 4 or greater. In some instances, the DAR ratio of the polynucleic acid molecule (B) to binding moiety A is about 5 or greater. In some instances, the DAR ratio of the polynucleic acid molecule (B) to binding moiety A is about 6 or greater. In some instances, the DAR ratio of the polynucleic acid molecule (B) to binding moiety A is about 7 or greater. In some instances, the DAR ratio of the polynucleic acid molecule (B) to binding moiety A is about 8 or greater. In some instances, the DAR ratio of the polynucleic acid molecule (B) to binding moiety A is about 9 or greater. In some instances, the DAR ratio of the polynucleic acid molecule (B) to binding moiety A is about 10 or greater. In some instances, the DAR ratio of the polynucleic acid molecule (B) to binding moiety A is about 11 or greater. In some instances, the DAR ratio of the polynucleic acid molecule (B) to binding moiety A is about 12 or greater.

In some instances, the DAR ratio of the polynucleic acid molecule (B) to binding moiety A (e.g., an antibody or binding fragment thereof) is about 1, 2, 3, 4, 5, 6, 7, 8, 9, 10, 11, 12, 13, 14, 15, or 16. In some instances, the DAR ratio of the polynucleic acid molecule (B) to binding moiety A is about 1. In some instances, the DAR ratio of the polynucleic acid molecule (B) to binding moiety A is about 2. In some instances, the DAR ratio of the polynucleic acid molecule (B) to binding moiety A is about 3. In some instances, the DAR ratio of the polynucleic acid molecule (B) to binding moiety A is about 4. In some instances, the DAR ratio of the polynucleic acid molecule (B) to binding moiety A is about 5. In some instances, the DAR ratio of the polynucleic acid molecule (B) to binding moiety A is about 6. In some instances, the DAR ratio of the polynucleic acid molecule (B) to binding moiety A is about 7. In some instances, the DAR ratio of the polynucleic acid molecule (B) to binding moiety A is about 8. In some instances, the DAR ratio of the polynucleic acid molecule (B) to binding moiety A is about 9. In some instances, the DAR ratio of the polynucleic acid molecule (B) to binding moiety A is about 10. In some instances, the DAR ratio of the polynucleic acid molecule (B) to binding moiety A is about 11. In some instances, the DAR ratio of the polynucleic acid molecule (B) to binding moiety A is about 12. In some instances, the DAR ratio of the polynucleic acid molecule (B) to binding moiety A is about 13. In some instances, the DAR ratio of the polynucleic acid molecule (B) to binding moiety A is about 14. In some instances, the DAR ratio of the polynucleic acid molecule (B) to binding moiety A is about 15. In some instances, the DAR ratio of the polynucleic acid molecule (B) to binding moiety A is about 16.

In some instances, the DAR ratio of the polynucleic acid molecule (B) to binding moiety A is 1, 2, 3, 4, 5, 6, 7, 8, 9, 10, 11, 12, 13, 14, 15, or 16. In some instances, the DAR ratio of the polynucleic acid molecule (B) to binding moiety A is 1. In some instances, the DAR ratio of the polynucleic acid molecule (B) to binding moiety A is 2. In some instances, the DAR ratio of the polynucleic acid molecule (B) to binding moiety A is 4. In some instances, the DAR ratio of the polynucleic acid molecule (B) to binding moiety A is 6. In some instances, the DAR ratio of the polynucleic acid molecule (B) to binding moiety A is 8. In some instances, the DAR ratio of the polynucleic acid molecule (B) to binding moiety A is 12.

In some embodiments, an antibody or its binding fragment is further modified using conventional techniques known in the art, for example, by using amino acid deletion, insertion, substitution, addition, and/or by recombination and/or any other modification (e.g. posttranslational and chemical modifications, such as glycosylation and phosphorylation) known in the art either alone or in combination. In some instances, the modification further comprises a modification for modulating interaction with Fc receptors. In some instances, the one or more modifications include those described in, for example, International Publication No. WO97/34631, which discloses amino acid residues involved in the interaction between the Fc domain and the FcRn receptor. Methods for introducing such modifications in the nucleic acid sequence underlying the amino acid sequence of an antibody or its binding fragment is well known to the person skilled in the art.

In some instances, an antibody binding fragment further encompasses its derivatives and includes polypeptide sequences containing at least one CDR.

In some instances, the term “single-chain” as used herein means that the first and second domains of a bi-specific single chain construct are covalently linked, preferably in the form of a co-linear amino acid sequence encodable by a single nucleic acid molecule.

In some instances, a bispecific single chain antibody construct relates to a construct comprising two antibody derived binding domains. In such embodiments, bi-specific single chain antibody construct is tandem bi-scFv or diabody. In some instances, a scFv contains a VH and VL domain connected by a linker peptide. In some instances, linkers are of a length and sequence sufficient to ensure that each of the first and second domains can, independently from one another, retain their differential binding specificities.

In some embodiments, binding to or interacting with as used herein defines a binding/interaction of at least two antigen-interaction-sites with each other. In some instances, antigen-interaction-site defines a motif of a polypeptide that shows the capacity of specific interaction with a specific antigen or a specific group of antigens. In some cases, the binding/interaction is also understood to define a specific recognition. In such cases, specific recognition refers to that the antibody or its binding fragment is capable of specifically interacting with and/or binding to at least two amino acids of each of a target molecule. For example, specific recognition relates to the specificity of the antibody molecule, or to its ability to discriminate between the specific regions of a target molecule. In additional instances, the specific interaction of the antigen-interaction-site with its specific antigen results in an initiation of a signal, e.g. due to the induction of a change of the conformation of the antigen, an oligomerization of the antigen, etc. In further embodiments, the binding is exemplified by the specificity of a “key-lock-principle”. Thus in some instances, specific motifs in the amino acid sequence of the antigen-interaction-site and the antigen bind to each other as a result of their primary, secondary or tertiary structure as well as the result of secondary modifications of said structure. In such cases, the specific interaction of the antigen-interaction-site with its specific antigen results as well in a simple binding of the site to the antigen.

In some instances, specific interaction further refers to a reduced cross-reactivity of the antibody or its binding fragment or a reduced off-target effect. For example, the antibody or its binding fragment that bind to the polypeptide/protein of interest but do not or do not essentially bind to any of the other polypeptides are considered as specific for the polypeptide/protein of interest. Examples for the specific interaction of an antigen-interaction-site with a specific antigen comprise the specificity of a ligand for its receptor, for example, the interaction of an antigenic determinant (epitope) with the antigenic binding site of an antibody.

### Additional Binding Moieties

In some embodiments, the binding moiety is a plasma protein. In some instances, the plasma protein comprises albumin. In some instances, the binding moiety A is albumin. In some instances, albumin is conjugated by one or more of a conjugation chemistry described herein to a polynucleic acid molecule. In some instances, albumin is conjugated by native ligation chemistry to a polynucleic acid molecule. In some instances, albumin is conjugated by lysine conjugation to a polynucleic acid molecule.

In some instances, the binding moiety is a steroid. Exemplary steroids include cholesterol, phospholipids, di- and triacylglycerols, fatty acids, hydrocarbons that are saturated, unsaturated, comprise substitutions, or combinations thereof. In some instances, the steroid is cholesterol. In some instances, the binding moiety is cholesterol. In some instances, cholesterol is conjugated by one or more of a conjugation chemistry described herein to a polynucleic acid molecule. In some instances, cholesterol is conjugated by native ligation chemistry to a polynucleic acid molecule. In some instances, cholesterol is conjugated by lysine conjugation to a polynucleic acid molecule.

In some instances, the binding moiety is a polymer, including but not limited to poly nucleic acid molecule aptamers that bind to specific surface markers on cells. In this instance the binding moiety is a polynucleic acid that does not hybridize to a target gene or mRNA, but instead is capable of selectively binding to a cell surface marker similarly to an antibody binding to its specific epitope of a cell surface marker.

In some cases, the binding moiety is a peptide. In some cases, the peptide comprises between about 1 and about 3 kDa. In some cases, the peptide comprises between about 1.2 and about 2.8 kDa, about 1.5 and about 2.5 kDa, or about 1.5 and about 2 kDa. In some instances, the peptide is a bicyclic peptide. In some cases, the bicyclic peptide is a constrained bicyclic peptide. In some instances, the binding moiety is a bicyclic peptide (e.g., bicycles from Bicycle Therapeutics).

In additional cases, the binding moiety is a small molecule. In some instances, the small molecule is an antibody-recruiting small molecule. In some cases, the antibody-recruiting small molecule comprises a target-binding terminus and an antibody-binding terminus, in which the target-binding terminus is capable of recognizing and interacting with a cell surface receptor. For example, in some instances, the target-binding terminus comprising a glutamate urea compound enables interaction with PSMA, thereby, enhances an antibody interaction with a cell (e.g., a cancerous cell) that expresses PSMA. In some instances, a binding moiety is a small molecule described in Zhang et al., “A remote arene-binding site on prostate specific membrane antigen revealed by antibody-recruiting small molecules,” J Am Chem Soc. 132(36): 12711-12716 (2010); or McEnaney, et al., “Antibody-recruiting molecules: an emerging paradigm for engaging immune function in treating human disease,” ACS Chem Biol. 7(7): 1139-1151 (2012).

### Production of Antibodies or Binding Fragments Thereof

In some embodiments, polypeptides described herein (e.g., antibodies and its binding fragments) are produced using any method known in the art to be useful for the synthesis of polypeptides (e.g., antibodies), in particular, by chemical synthesis or by recombinant expression, and are preferably produced by recombinant expression techniques.

In some instances, an antibody or its binding fragment thereof is expressed recombinantly, and the nucleic acid encoding the antibody or its binding fragment is assembled from chemically synthesized oligonucleotides (e.g., as described in Kutmeier et al., 1994, BioTechniques 17:242), which involves the synthesis of overlapping oligonucleotides containing portions of the sequence encoding the antibody, annealing and ligation of those oligonucleotides, and then amplification of the ligated oligonucleotides by PCR.

Alternatively, a nucleic acid molecule encoding an antibody is optionally generated from a suitable source (e.g., an antibody cDNA library, or cDNA library generated from any tissue or cells expressing the immunoglobulin) by PCR amplification using synthetic primers hybridizable to the 3′ and 5′ ends of the sequence or by cloning using an oligonucleotide probe specific for the particular gene sequence.

In some instances, an antibody or its binding is optionally generated by immunizing an animal, such as a rabbit, to generate polyclonal antibodies or, more preferably, by generating monoclonal antibodies, e.g., as described by Kohler and Milstein (1975, Nature 256:495-497) or, as described by Kozbor et al. (1983, Immunology Today 4:72) or Cole et al. (1985 in Monoclonal Antibodies and Cancer Therapy, Alan R. Liss, Inc., pp. 77-96). Alternatively, a clone encoding at least the Fab portion of the antibody is optionally obtained by screening Fab expression libraries (e.g., as described in Huse et al., 1989, Science 246:1275-1281) for clones of Fab fragments that bind the specific antigen or by screening antibody libraries (See, e.g., Clackson et al., 1991, Nature 352:624; Hane et al., 1997 Proc. Natl. Acad. Sci. USA 94:4937).

In some embodiments, techniques developed for the production of “chimeric antibodies” (Morrison et al., 1984, Proc. Natl. Acad. Sci. 81:851-855; Neuberger et al., 1984, Nature 312:604-608; Takeda et al., 1985, Nature 314:452-454) by splicing genes from a mouse antibody molecule of appropriate antigen specificity together with genes from a human antibody molecule of appropriate biological activity are used. A chimeric antibody is a molecule in which different portions are derived from different animal species, such as those having a variable region derived from a murine monoclonal antibody and a human immunoglobulin constant region, e.g., humanized antibodies.

In some embodiments, techniques described for the production of single chain antibodies (U.S. Pat. No. 4,694,778; Bird, 1988, Science 242:423-42; Huston et al., 1988, Proc. Natl. Acad. Sci. USA 85:5879-5883; and Ward et al., 1989, Nature 334:544-54) are adapted to produce single chain antibodies. Single chain antibodies are formed by linking the heavy and light chain fragments of the Fv region via an amino acid bridge, resulting in a single chain polypeptide. Techniques for the assembly of functional Fv fragments in E. coli are also optionally used (Skerra et al., 1988, Science 242:1038-1041).

In some embodiments, an expression vector comprising the nucleotide sequence of an antibody or the nucleotide sequence of an antibody is transferred to a host cell by conventional techniques (e.g., electroporation, liposomal transfection, and calcium phosphate precipitation), and the transfected cells are then cultured by conventional techniques to produce the antibody. In specific embodiments, the expression of the antibody is regulated by a constitutive, an inducible or a tissue, specific promoter.

In some embodiments, a variety of host-expression vector systems is utilized to express an antibody or its binding fragment described herein. Such host-expression systems represent vehicles by which the coding sequences of the antibody is produced and subsequently purified, but also represent cells that are, when transformed or transfected with the appropriate nucleotide coding sequences, express an antibody or its binding fragment in situ. These include, but are not limited to, microorganisms such as bacteria (e.g., E. coli and B. subtilis) transformed with recombinant bacteriophage DNA, plasmid DNA or cosmid DNA expression vectors containing an antibody or its binding fragment coding sequences; yeast (e.g., Saccharomyces Pichia) transformed with recombinant yeast expression vectors containing an antibody or its binding fragment coding sequences; insect cell systems infected with recombinant virus expression vectors (e.g., baculovirus) containing an antibody or its binding fragment coding sequences; plant cell systems infected with recombinant virus expression vectors (e.g., cauliflower mosaic virus (CaMV) and tobacco mosaic virus (TMV)) or transformed with recombinant plasmid expression vectors (e.g., Ti plasmid) containing an antibody or its binding fragment coding sequences; or mammalian cell systems (e.g., COS, CHO, BH, 293, 293T, 3T3 cells) harboring recombinant expression constructs containing promoters derived from the genome of mammalian cells (e.g., metallothionein promoter) or from mammalian viruses (e.g. the adenovirus late promoter; the vaccinia virus 7.5K promoter).

For long-term, high-yield production of recombinant proteins, stable expression is preferred. In some instances, cell lines that stably express an antibody are optionally engineered. Rather than using expression vectors that contain viral origins of replication, host cells are transformed with DNA controlled by appropriate expression control elements (e.g., promoter, enhancer, sequences, transcription terminators, polyadenylation sites, etc.), and a selectable marker. Following the introduction of the foreign DNA, engineered cells are then allowed to grow for 1-2 days in an enriched media, and then are switched to a selective media. The selectable marker in the recombinant plasmid confers resistance to the selection and allows cells to stably integrate the plasmid into their chromosomes and grow to form foci that in turn are cloned and expanded into cell lines. This method can advantageously be used to engineer cell lines which express the antibody or its binding fragments.

In some instances, a number of selection systems are used, including but not limited to the herpes simplex virus thymidine kinase (Wigler et al., 1977, Cell 11:223), hypoxanthine-guanine phosphoribosyltransferase (Szybalska & Szybalski, 192, Proc. Natl. Acad. Sci. USA 48:202), and adenine phosphoribosyltransferase (Lowy et al., 1980, Cell 22:817) genes are employed in tk-, hgprt- or aprt-cells, respectively. Also, antimetabolite resistance are used as the basis of selection for the following genes: dhfr, which confers resistance to methotrexate (Wigler et al., 1980, Proc. Natl. Acad. Sci. USA 77:357; O'Hare et al., 1981, Proc. Natl. Acad. Sci. USA 78:1527); gpt, which confers resistance to mycophenolic acid (Mulligan & Berg, 1981, Proc. Natl. Acad. Sci. USA 78:2072); neo, which confers resistance to the aminoglycoside G-418 (Clinical Pharmacy 12:488-505; Wu and Wu, 1991, Biotherapy 3:87-95; Tolstoshev, 1993, Ann. Rev. Pharmacol. Toxicol. 32:573-596; Mulligan, 1993, Science 260:926-932; and Morgan and Anderson, 1993, Ann. Rev. Biochem. 62:191-217; May, 1993, TIB TECH 11(5):155-215) and hygro, which confers resistance to hygromycin (Santerre et al., 1984, Gene 30:147). Methods commonly known in the art of recombinant DNA technology which can be used are described in Ausubel et al. (eds., 1993, Current Protocols in Molecular Biology, John Wiley & Sons, NY; Kriegler, 1990, Gene Transfer and Expression, A Laboratory Manual, Stockton Press, NY; and in Chapters 12 and 13, Dracopoli et al. (eds), 1994, Current Protocols in Human Genetics, John Wiley & Sons, NY.; Colberre-Garapin et al., 1981, J. Mol. Biol. 150:1).

In some instances, the expression levels of an antibody are increased by vector amplification (for a review, see Bebbington and Hentschel, The use of vectors based on gene amplification for the expression of cloned genes in mammalian cells in DNA cloning, Vol. 3. (Academic Press, New York, 1987)). When a marker in the vector system expressing an antibody is amplifiable, an increase in the level of inhibitor present in culture of host cell will increase the number of copies of the marker gene. Since the amplified region is associated with the nucleotide sequence of the antibody, production of the antibody will also increase (Crouse et al., 1983, Mol. Cell Biol. 3:257).

In some instances, any method known in the art for purification of an antibody is used, for example, by chromatography (e.g., ion exchange, affinity, particularly by affinity for the specific antigen after Protein A, and sizing column chromatography), centrifugation, differential solubility, or by any other standard technique for the purification of proteins.

### Polymer Conjugating Moiety

In some embodiments, a polymer moiety C is further conjugated to a polynucleic acid molecule described herein, a binding moiety described herein, or in combinations thereof. In some instances, a polymer moiety C is conjugated a polynucleic acid molecule. In some cases, a polymer moiety C is conjugated to a binding moiety. In other cases, a polymer moiety C is conjugated to a polynucleic acid molecule-binding moiety molecule. In additional cases, a polymer moiety C is conjugated, and as discussed under the Therapeutic Molecule Platform section.

In some instances, the polymer moiety C is a natural or synthetic polymer, consisting of long chains of branched or unbranched monomers, and/or cross-linked network of monomers in two or three dimensions. In some instances, the polymer moiety C includes a polysaccharide, lignin, rubber, or polyalkylen oxide (e.g., polyethylene glycol). In some instances, the at least one polymer moiety C includes, but is not limited to, alpha-, omega-dihydroxylpolyethyleneglycol, biodegradable lactone-based polymer, e.g. polyacrylic acid, polylactide acid (PLA), poly(glycolic acid) (PGA), polypropylene, polystyrene, polyolefin, polyamide, polycyanoacrylate, polyimide, polyethylenterephthalat (PET, PETG), polyethylene terephthalate (PETE), polytetramethylene glycol (PTG), or polyurethane as well as mixtures thereof. As used herein, a mixture refers to the use of different polymers within the same compound as well as in reference to block copolymers. In some cases, block copolymers are polymers wherein at least one section of a polymer is build up from monomers of another polymer. In some instances, the polymer moiety C comprises polyalkylene oxide. In some instances, the polymer moiety C comprises PEG. In some instances, the polymer moiety C comprises polyethylene imide (PEI) or hydroxy ethyl starch (HES).

In some instances, C is a PEG moiety. In some instances, the PEG moiety is conjugated at the 5′ terminus of the polynucleic acid molecule while the binding moiety is conjugated at the 3′ terminus of the polynucleic acid molecule. In some instances, the PEG moiety is conjugated at the 3′ terminus of the polynucleic acid molecule while the binding moiety is conjugated at the 5′ terminus of the polynucleic acid molecule. In some instances, the PEG moiety is conjugated to an internal site of the polynucleic acid molecule. In some instances, the PEG moiety, the binding moiety, or a combination thereof, are conjugated to an internal site of the polynucleic acid molecule. In some instances, the conjugation is a direct conjugation. In some instances, the conjugation is via native ligation.

In some embodiments, the polyalkylene oxide (e.g., PEG) is a polydispers or monodispers compound. In some instances, polydispers material comprises disperse distribution of different molecular weight of the material, characterized by mean weight (weight average) size and dispersity. In some instances, the monodisperse PEG comprises one size of molecules. In some embodiments, C is poly- or monodispersed polyalkylene oxide (e.g., PEG) and the indicated molecular weight represents an average of the molecular weight of the polyalkylene oxide, e.g., PEG, molecules.

In some embodiments, the molecular weight of the polyalkylene oxide (e.g., PEG) is about 200, 300, 400, 500, 600, 700, 800, 900, 1000, 1100, 1200, 1300, 1400, 1450, 1500, 1600, 1700, 1800, 1900, 2000, 2100, 2200, 2300, 2400, 2500, 2600, 2700, 2800, 2900, 3000, 3250, 3350, 3500, 3750, 4000, 4250, 4500, 4600, 4750, 5000, 5500, 6000, 6500, 7000, 7500, 8000, 10,000, 12,000, 20,000, 35,000, 40,000, 50,000, 60,000, or 100,000 Da.

In some embodiments, C is polyalkylene oxide (e.g., PEG) and has a molecular weight of about 200, 300, 400, 500, 600, 700, 800, 900, 1000, 1100, 1200, 1300, 1400, 1450, 1500, 1600, 1700, 1800, 1900, 2000, 2100, 2200, 2300, 2400, 2500, 2600, 2700, 2800, 2900, 3000, 3250, 3350, 3500, 3750, 4000, 4250, 4500, 4600, 4750, 5000, 5500, 6000, 6500, 7000, 7500, 8000, 10,000, 12,000, 20,000, 35,000, 40,000, 50,000, 60,000, or 100,000 Da. In some embodiments, C is PEG and has a molecular weight of about 200, 300, 400, 500, 600, 700, 800, 900, 1000, 1100, 1200, 1300, 1400, 1450, 1500, 1600, 1700, 1800, 1900, 2000, 2100, 2200, 2300, 2400, 2500, 2600, 2700, 2800, 2900, 3000, 3250, 3350, 3500, 3750, 4000, 4250, 4500, 4600, 4750, 5000, 5500, 6000, 6500, 7000, 7500, 8000, 10,000, 12,000, 20,000, 35,000, 40,000, 50,000, 60,000, or 100,000 Da. In some instances, the molecular weight of C is about 200 Da. In some instances, the molecular weight of C is about 300 Da. In some instances, the molecular weight of C is about 400 Da. In some instances, the molecular weight of C is about 500 Da. In some instances, the molecular weight of C is about 600 Da. In some instances, the molecular weight of C is about 700 Da. In some instances, the molecular weight of C is about 800 Da. In some instances, the molecular weight of C is about 900 Da. In some instances, the molecular weight of C is about 1000 Da. In some instances, the molecular weight of C is about 1100 Da. In some instances, the molecular weight of C is about 1200 Da. In some instances, the molecular weight of C is about 1300 Da. In some instances, the molecular weight of C is about 1400 Da. In some instances, the molecular weight of C is about 1450 Da. In some instances, the molecular weight of C is about 1500 Da. In some instances, the molecular weight of C is about 1600 Da. In some instances, the molecular weight of C is about 1700 Da. In some instances, the molecular weight of C is about 1800 Da. In some instances, the molecular weight of C is about 1900 Da. In some instances, the molecular weight of C is about 2000 Da. In some instances, the molecular weight of C is about 2100 Da. In some instances, the molecular weight of C is about 2200 Da. In some instances, the molecular weight of C is about 2300 Da. In some instances, the molecular weight of C is about 2400 Da. In some instances, the molecular weight of C is about 2500 Da. In some instances, the molecular weight of C is about 2600 Da. In some instances, the molecular weight of C is about 2700 Da. In some instances, the molecular weight of C is about 2800 Da. In some instances, the molecular weight of C is about 2900 Da. In some instances, the molecular weight of C is about 3000 Da. In some instances, the molecular weight of C is about 3250 Da. In some instances, the molecular weight of C is about 3350 Da. In some instances, the molecular weight of C is about 3500 Da. In some instances, the molecular weight of C is about 3750 Da. In some instances, the molecular weight of C is about 4000 Da. In some instances, the molecular weight of C is about 4250 Da. In some instances, the molecular weight of C is about 4500 Da. In some instances, the molecular weight of C is about 4600 Da. In some instances, the molecular weight of C is about 4750 Da. In some instances, the molecular weight of C is about 5000 Da. In some instances, the molecular weight of C is about 5500 Da. In some instances, the molecular weight of C is about 6000 Da. In some instances, the molecular weight of C is about 6500 Da. In some instances, the molecular weight of C is about 7000 Da. In some instances, the molecular weight of C is about 7500 Da. In some instances, the molecular weight of C is about 8000 Da. In some instances, the molecular weight of C is about 10,000 Da. In some instances, the molecular weight of C is about 12,000 Da. In some instances, the molecular weight of C is about 20,000 Da. In some instances, the molecular weight of C is about 35,000 Da. In some instances, the molecular weight of C is about 40,000 Da. In some instances, the molecular weight of C is about 50,000 Da. In some instances, the molecular weight of C is about 60,000 Da. In some instances, the molecular weight of C is about 100,000 Da.

In some embodiments, the polyalkylene oxide (e.g., PEG) is a discrete PEG, in which the discrete PEG is a polymeric PEG comprising more than one repeating ethylene oxide units. In some instances, a discrete PEG (dPEG) comprises from 2 to 60, from 2 to 50, or from 2 to 48 repeating ethylene oxide units. In some instances, a dPEG comprises about 2, 3, 4, 5, 6, 7, 8, 9, 10, 11, 12, 13, 14, 15, 16, 17, 18, 19, 20, 22, 24, 26, 28, 30, 35, 40, 42, 48, 50 or more repeating ethylene oxide units. In some instances, a dPEG comprises about 2 or more repeating ethylene oxide units. In some instances, a dPEG comprises about 3 or more repeating ethylene oxide units. In some instances, a dPEG comprises about 4 or more repeating ethylene oxide units. In some instances, a dPEG comprises about 5 or more repeating ethylene oxide units. In some instances, a dPEG comprises about 6 or more repeating ethylene oxide units. In some instances, a dPEG comprises about 7 or more repeating ethylene oxide units. In some instances, a dPEG comprises about 8 or more repeating ethylene oxide units. In some instances, a dPEG comprises about 9 or more repeating ethylene oxide units. In some instances, a dPEG comprises about 10 or more repeating ethylene oxide units. In some instances, a dPEG comprises about 11 or more repeating ethylene oxide units. In some instances, a dPEG comprises about 12 or more repeating ethylene oxide units. In some instances, a dPEG comprises about 13 or more repeating ethylene oxide units. In some instances, a dPEG comprises about 14 or more repeating ethylene oxide units. In some instances, a dPEG comprises about 15 or more repeating ethylene oxide units. In some instances, a dPEG comprises about 16 or more repeating ethylene oxide units. In some instances, a dPEG comprises about 17 or more repeating ethylene oxide units. In some instances, a dPEG comprises about 18 or more repeating ethylene oxide units. In some instances, a dPEG comprises about 19 or more repeating ethylene oxide units. In some instances, a dPEG comprises about 20 or more repeating ethylene oxide units. In some instances, a dPEG comprises about 22 or more repeating ethylene oxide units. In some instances, a dPEG comprises about 24 or more repeating ethylene oxide units. In some instances, a dPEG comprises about 26 or more repeating ethylene oxide units. In some instances, a dPEG comprises about 28 or more repeating ethylene oxide units. In some instances, a dPEG comprises about 30 or more repeating ethylene oxide units. In some instances, a dPEG comprises about 35 or more repeating ethylene oxide units. In some instances, a dPEG comprises about 40 or more repeating ethylene oxide units. In some instances, a dPEG comprises about 42 or more repeating ethylene oxide units. In some instances, a dPEG comprises about 48 or more repeating ethylene oxide units. In some instances, a dPEG comprises about 50 or more repeating ethylene oxide units. In some cases, a dPEG is synthesized as a single molecular weight compound from pure (e.g., about 95%, 98%, 99%, or 99.5%) staring material in a step-wise fashion. In some cases, a dPEG has a specific molecular weight, rather than an average molecular weight. In some cases, a dPEG described herein is a dPEG from Quanta Biodesign, LMD.

In some embodiments, the polymer moiety C comprises a cationic mucic acid-based polymer (cMAP). In some instances, cMPA comprises one or more subunit of at least one repeating subunit, and the subunit structure is represented as Formula (III):

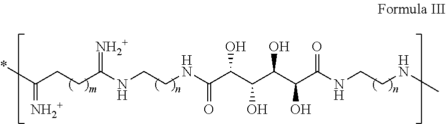

wherein m is independently at each occurrence 1, 2, 3, 4, 5, 6, 7, 8, 9, or 10, preferably 4-6 or 5; and n is independently at each occurrence 1, 2, 3, 4, or 5. In some embodiments, m and n are, for example, about 10.

In some instances, cMAP is further conjugated to a PEG moiety, generating a cMAP-PEG copolymer, an mPEG-cMAP-PEGm triblock polymer, or a cMAP-PEG-cMAP triblock polymer. In some instances, the PEG moiety is in a range of from about 500 Da to about 50,000 Da. In some instances, the PEG moiety is in a range of from about 500 Da to about 1000 Da, greater than 1000 Da to about 5000 Da, greater than 5000 Da to about 10,000 Da, greater than 10,000 to about 25,000 Da, greater than 25,000 Da to about 50,000 Da, or any combination of two or more of these ranges.

In some instances, the polymer moiety C is cMAP-PEG copolymer, an mPEG-cMAP-PEGm triblock polymer, or a cMAP-PEG-cMAP triblock polymer. In some cases, the polymer moiety C is cMAP-PEG copolymer. In other cases, the polymer moiety C is an mPEG-cMAP-PEGm triblock polymer. In additional cases, the polymer moiety C is a cMAP-PEG-cMAP triblock polymer.

In some embodiments, the polymer moiety C is conjugated to the polynucleic acid molecule, the binding moiety, and optionally to the endosomolytic moiety.

### Endosomolytic Moiety

In some embodiments, a molecule of Formula (I): A-X—B—Y—C, further comprises an additional conjugating moiety. In some instances, the additional conjugating moiety is an endosomolytic moiety. In some cases, the endosomolytic moiety is a cellular compartmental release component, such as a compound capable of releasing from any of the cellular compartments known in the art, such as the endosome, lysosome, endoplasmic reticulum (ER), golgi apparatus, microtubule, peroxisome, or other vesicular bodies with the cell. In some cases, the endosomolytic moiety comprises an endosomolytic polypeptide, an endosomolytic polymer, an endosomolytic lipid, or an endosomolytic small molecule. In some cases, the endosomolytic moiety comprises an endosomolytic polypeptide. In other cases, the endosomolytic moiety comprises an endosomolytic polymer.

### Endosomolytic Polypeptides

In some embodiments, a molecule of Formula (I): A-X—B—Y—C, is further conjugated with an endosomolytic polypeptide. In some cases, the endosomolytic polypeptide is a pH-dependent membrane active peptide. In some cases, the endosomolytic polypeptide is an amphipathic polypeptide. In additional cases, the endosomolytic polypeptide is a peptidomimetic. In some instances, the endosomolytic polypeptide comprises INF, melittin, meucin, or their respective derivatives thereof. In some instances, the endosomolytic polypeptide comprises INF or its derivatives thereof. In other cases, the endosomolytic polypeptide comprises melittin or its derivatives thereof. In additional cases, the endosomolytic polypeptide comprises meucin or its derivatives thereof.

In some instances, INF7 is a 24 residue polypeptide those sequence comprises CGIFGEIEELIEEGLENLIDWGNA (SEQ ID NO: 1243), or GLFEAIEGFIENGWEGMIDGWYGC (SEQ ID NO: 1244). In some instances, INF7 or its derivatives comprise a sequence of:

(SEQ ID NO: 1245)
GLFEAIEGFIENGWEGMIWDYGSGSCG
(SEQ ID NO: 1246)
GLFEAIEGFIENGWEGMIDG WYG-(PEG)6-NH2,
or
(SEQ ID NO: 1247)
GLFEAIEGFIENGWEGMIWDYG-SGSC-K(GalNAc)2.

In some cases, melittin is a 26 residue polypeptide those sequence comprises CLIGAILKVLATGLPTLISWIKNKRKQ (SEQ ID NO: 1248), or GIGAVLKVLTTGLPALISWIKRKRQQ (SEQ ID NO: 1249). In some instances, melittin comprises a polypeptide sequence as described in U.S. Pat. No. 8,501,930.

In some instances, meucin is an antimicrobial peptide (AMP) derived from the venom gland of the scorpion Mesobuthus eupeus. In some instances, meucin comprises of meucin-13 those sequence comprises IFGAIAGLLKNIF-NH₂ (SEQ ID NO: 1250) and meucin-18 those sequence comprises FFGHLFKLATKIIPSLFQ (SEQ ID NO: 1251).

In some instances, the endosomolytic polypeptide comprises a polypeptide in which its sequence is at least 50%, 60%, 70%, 80%, 90%, 95%, or 99% sequence identity to INF7 or its derivatives thereof, melittin or its derivatives thereof, or meucin or its derivatives thereof. In some instances, the endosomolytic moiety comprises INF7 or its derivatives thereof, melittin or its derivatives thereof, or meucin or its derivatives thereof.

In some instances, the endosomolytic moiety is INF7 or its derivatives thereof. In some cases, the endosomolytic moiety comprises a polypeptide having at least 50%, 55%, 60%, 65%, 70%, 75%, 80%, 85%, 90%, 95%, 96%, 97%, 98%, 99%, or 100% sequence identity to SEQ ID NOs: 1243-1247. In some cases, the endosomolytic moiety comprises a polypeptide having at least 50%, 55%, 60%, 65%, 70%, 75%, 80%, 85%, 90%, 95%, 96%, 97%, 98%, 99%, or 100% sequence identity to SEQ ID NO: 1243. In some cases, the endosomolytic moiety comprises a polypeptide having at least 50%, 55%, 60%, 65%, 70%, 75%, 80%, 85%, 90%, 95%, 96%, 97%, 98%, 99%, or 100% sequence identity to SEQ ID NO: 1244-1247. In some cases, the endosomolytic moiety comprises SEQ ID NO: 1243. In some cases, the endosomolytic moiety comprises SEQ ID NO: 1244-1247. In some cases, the endosomolytic moiety consists of SEQ ID NO: 1243. In some cases, the endosomolytic moiety consists of SEQ ID NO: 1244-1247.

In some instances, the endosomolytic moiety is melittin or its derivatives thereof. In some cases, the endosomolytic moiety comprises a polypeptide having at least 50%, 55%, 60%, 65%, 70%, 75%, 80%, 85%, 90%, 95%, 96%, 97%, 98%, 99%, or 100% sequence identity to SEQ ID NOs: 1248 or 1249. In some cases, the endosomolytic moiety comprises a polypeptide having at least 50%, 55%, 60%, 65%, 70%, 75%, 80%, 85%, 90%, 95%, 96%, 97%, 98%, 99%, or 100% sequence identity to SEQ ID NO: 1248. In some cases, the endosomolytic moiety comprises a polypeptide having at least 50%, 55%, 60%, 65%, 70%, 75%, 80%, 85%, 90%, 95%, 96%, 97%, 98%, 99%, or 100% sequence identity to SEQ ID NO: 1249. In some cases, the endosomolytic moiety comprises SEQ ID NO: 1248. In some cases, the endosomolytic moiety comprises SEQ ID NO: 1249. In some cases, the endosomolytic moiety consists of SEQ ID NO: 1248. In some cases, the endosomolytic moiety consists of SEQ ID NO: 1249.

In some instances, the endosomolytic moiety is meucin or its derivatives thereof. In some cases, the endosomolytic moiety comprises a polypeptide having at least 50%, 55%, 60%, 65%, 70%, 75%, 80%, 85%, 90%, 95%, 96%, 97%, 98%, 99%, or 100% sequence identity to SEQ ID NOs: 1250 or 1251. In some cases, the endosomolytic moiety comprises a polypeptide having at least 50%, 55%, 60%, 65%, 70%, 75%, 80%, 85%, 90%, 95%, 96%, 97%, 98%, 99%, or 100% sequence identity to SEQ ID NO: 1250. In some cases, the endosomolytic moiety comprises a polypeptide having at least 50%, 55% 60%, 65%, 70%, 75%, 80%, 85%, 90%, 95%, 96%, 97%, 98%, 99%, or 100% sequence identity to SEQ ID NO: 1251. In some cases, the endosomolytic moiety comprises SEQ ID NO: 1250. In some cases, the endosomolytic moiety comprises SEQ ID NO: 1251. In some cases, the endosomolytic moiety consists of SEQ ID NO: 1250. In some cases, the endosomolytic moiety consists of SEQ ID NO: 1251.

In some instances, the endosomolytic moiety comprises a sequence as illustrated in Table 10.

TABLE 10

| | | | SEQ | |
|---|---|---|---|---|
| | | | ID | |
| Name | Origin | Amino Acid Sequence | NO: | Type |
| Pep-1 | NLS from Simian Virus | KETWWETWWTEWSQPKKKRKV | 1252 | Primary |
| | 40 large antigen and | | | amphipathic |
| | Reverse transcriptase of | | | |
| | HIV | | | |
| pVEC | VE-cadherin | LLIILRRRRIRKQAHAHSK | 1253 | Primary |
| | | | | amphipathic |
| VT5 | Synthetic peptide | DPKGDPKGVTVTVTVTVTGKGDP | 1254 | β-sheet |
| | | KPD | | amphipathic |
| C105Y | 1-antitrypsin | CSIPPEVKFNKPFVYLI | 1255 | — |
| Transportan | Galanin and mastoparan | GWTLNSAGYLLGKINLKALAALA | 1256 | Primary |
| | | KKIL | | amphipathic |
| TP10 | Galanin and mastoparan | AGYLLGKINLKALAALAKKIL | 1257 | Primary |
| | | | | amphipathic |
| MPG | A hydrofobic domain | GALFLGFLGAAGSTMGA | 1258 | β-sheet |
| | from the fusion | | | amphipathic |
| | sequence of HIV gp41 | | | |
| | and NLS of SV40 T | | | |
| | antigen | | | |
| gH625 | Glycoprotein gH of | HGLASTLTRWAHYNALIRAF | 1259 | Secondary |
| | HSV type I | | | amphipathic |
| | | | | α-helical |
| CADY | PPTG1 peptide | GLWRALWRLLRSLWRLLWRA | 1260 | Secondary |
| | | | | amphipathic |
| | | | | α-helical |
| GALA | Synthetic peptide | WEAALAEALAEALAEHLAEALAE | 1261 | Secondary |
| | | ALEALAA | | amphipathic |
| | | | | α-helical |
| INF | Influenza HA2 fusion | GLFEAIEGFIENGWEGMIDGWYGC | 1262 | Secondary |
| | peptide | | | amphipathic |
| | | | | α-helical/ |
| | | | | pH- |
| | | | | dependent |
| | | | | membrane |
| | | | | active |
| | | | | peptide |
| HA2E5- | Influenza HA2 subunit | GLFGAIAGFIENGWEGMIDGWYG | 1263 | Secondary |
| TAT | of influenza virus X31 | | | amphipathic |
| | strain fusion peptide | | | α-helical/ |
| | | | | pH- |
| | | | | dependent |
| | | | | membrane |
| | | | | active |
| | | | | peptide |
| HA2- | Influenza HA2 subunit | GLFGAIAGFIENGWEGMIDGRQIKI | 1264 | pH- |
| penetratin | of influenza virus X31 | WFQNRRMKW | | dependent |
| | strain fusion peptide | KK-amide | | membrane |
| | | | | active |
| | | | | peptide |
| HA-K4 | Influenza HA2 subunit | GLFGAIAGFIENGWEGMIDG- | 1265 | pH- |
| | of influenza virus X31 | SSKKKK | | dependent |
| | strain fusion peptide | | | membrane |
| | | | | active |
| | | | | peptide |
| HA2E4 | Influenza HA2 subunit | GLFEAIAGFIENGWEGMIDGGGYC | 1266 | pH- |
| | of influenza virus X31 | | | dependent |
| | strain fusion peptide | | | membrane |
| | | | | active |
| | | | | peptide |
| H5WYG | HA2 analogue | GLFHAIAHFIHGGWH | 1267 | pH- |
| | | GLIHGWYG | | dependent |
| | | | | membrane |
| | | | | active |
| | | | | peptide |
| GALA- | INF3 fusion peptide | GLFEAIEGFIENGWEGLAEALAEAL | 1268 | pH- |
| INF3- | | EALAA- | | dependent |
| (PEG)6-NH | | (PEG)6-NH2 | | membrane |
| | | | | active |
| | | | | peptide |
| CM18- | Cecropin-A-Melittin₂₋₁₂ | KWKLFKKIGAVLKVLTTG- | 1269 | pH- |
| TAT11 | (CM₁₈) fusion peptide | YGRKKIMQRRR | | dependent |
| | | | | membrane |
| | | | | active |
| | | | | peptide |

In some cases, the endosomolytic moiety comprises a Bak BH3 polypeptide which induces apoptosis through antagonization of suppressor targets such as Bcl-2 and/or Bcl-x_{L}. In some instances, the endosomolytic moiety comprises a Bak BH3 polypeptide described in Albarran, et al., “Efficient intracellular delivery of a pro-apoptotic peptide with a pH-responsive carrier,” Reactive & Functional Polymers 71: 261-265 (2011).

In some instances, the endosomolytic moiety comprises a polypeptide (e.g., a cell-penetrating polypeptide) as described in PCT Publication Nos. WO2013/166155 or WO2015/069587.

### Endosomolytic Polymers

In some embodiments, a molecule of Formula (I): A-X—B—Y—C, is further conjugated with an endosomolytic polymer. As used herein, an endosomolytic polymer comprises a linear, a branched network, a star, a comb, or a ladder type of polymer. In some instances, an endosomolytic polymer is a homopolymer or a copolymer comprising two or more different types of monomers. In some cases, an endosomolytic polymer is a polycation polymer. In other cases, an endosomolytic polymer is a polyanion polymer.

In some instances, a polycation polymer comprises monomer units that are charge positive, charge neutral, or charge negative, with a net charge being positive. In other cases, a polycation polymer comprises a non-polymeric molecule that contains two or more positive charges. Exemplary cationic polymers include, but are not limited to, poly(L-lysine) (PLL), poly(L-arginine) (PLA), polyethyleneimine (PEI), poly[α-(4-aminobutyl)-L-glycolic acid](PAGA), 2-(dimethylamino)ethyl methacrylate (DMAEMA), or N,N-Diethylaminoethyl Methacrylate (DEAEMA).

In some cases, a polyanion polymer comprises monomer units that are charge positive, charge neutral, or charge negative, with a net charge being negative. In other cases, a polyanion polymer comprises a non-polymeric molecule that contains two or more negative charges. Exemplary anionic polymers include p(alkylacrylates) (e.g., poly(propyl acrylic acid) (PPAA)) or poly(N-isopropylacrylamide) (NIPAM). Additional examples include PP75, a L-phenylalanine-poly(L-lysine isophthalamide) polymer described in Khormaee, et al., “Edosomolytic anionic polymer for the cytoplasmic delivery of siRNAs in localized in vivo applications,” Advanced Functional Materials 23: 565-574 (2013).

In some embodiments, an endosomolytic polymer described herein is a pH-responsive endosomolytic polymer. A pH-responsive polymer comprises a polymer that increases in size (swell) or collapses depending on the pH of the environment. Polyacrylic acid and chitosan are examples of pH-responsive polymers.

In some instances, an endosomolytic moiety described herein is a membrane-disruptive polymer. In some cases, the membrane-disruptive polymer comprises a cationic polymer, a neutral or hydrophobic polymer, or an anionic polymer. In some instances, the membrane-disruptive polymer is a hydrophilic polymer.

In some instances, an endosomolytic moiety described herein is a pH-responsive membrane-disruptive polymer. Exemplary pH-responsive membrane-disruptive polymers include p(alkylacrylic acids), poly(N-isopropylacrylamide) (NIPAM) copolymers, succinylated p(glycidols), and p(β-malic acid) polymers.

In some instances, p(alkylacrylic acids) include poly(propylacrylic acid) (polyPAA), poly(methacrylic acid) (PMAA), poly(ethylacrylic acid) (PEAA), and poly(propyl acrylic acid) (PPAA). In some instances, a p(alkylacrylic acid) include a p(alkylacrylic acid) described in Jones, et al., Biochemistry Journal 372: 65-75 (2003).

In some embodiments, a pH-responsive membrane-disruptive polymer comprises p(butyl acrylate-co-methacrylic acid). (see Bulmus, et al., Journal of Controlled Release 93: 105-120 (2003); and Yessine, et al., Biochimica et Biophysica Acta 1613: 28-38 (2003))

In some embodiments, a pH-responsive membrane-disruptive polymer comprises p(styrene-alt-maleic anhydride). (see Henry, et al., Biomacromolecules 7: 2407-2414 (2006))

In some embodiments, a pH-responsive membrane-disruptive polymer comprises pyridyldisulfide acrylate (PDSA) polymers such as poly(MAA-co-PDSA), poly(EAA-co-PDSA), poly(PAA-co-PDSA), poly(MAA-co-BA-co-PDSA), poly(EAA-co-BA-co-PDSA), or poly(PAA-co-BA-co-PDSA) polymers. (see El-Sayed, et al., “Rational design of composition and activity correlations for pH-responsive and glutathione-reactive polymer therapeutics,” Journal of Controlled Release 104: 417-427 (2005); or Flanary et al., “Antigen delivery with poly(propylacrylic acid) conjugation enhanced MHC-1 presentation and T-cell activation,” Bioconjugate Chem. 20: 241-248 (2009))

In some embodiments, a pH-responsive membrane-disruptive polymer comprises a lytic polymer comprising the base structure of:

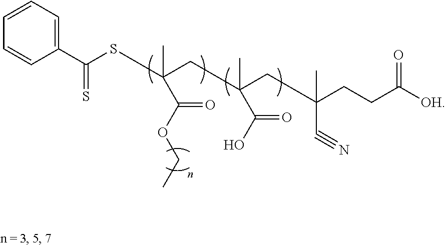

In some instances, an endosomolytic moiety described herein is further conjugated to an additional conjugate, e.g., a polymer (e.g., PEG), or a modified polymer (e.g., cholesterol-modified polymer).

In some instances, the additional conjugate comprises a detergent (e.g., Triton X-100). In some instances, an endosomolytic moiety described herein comprises a polymer (e.g., a poly(amidoamine)) conjugated with a detergent (e.g., Triton X-100). In some instances, an endosomolytic moiety described herein comprises poly(amidoamine)-Triton X-100 conjugate (Duncan, et al., “A polymer-Triton X-100 conjugate capable of pH-dependent red blood cell lysis: a model system illustrating the possibility of drug delivery within acidic intracellular compartments,” Journal of Drug Targeting 2: 341-347 (1994)).

### Endosomolytic Lipids

In some embodiments, the endosomolytic moiety is a lipid (e.g., a fusogenic lipid). In some embodiments, a molecule of Formula (I): A-X—B—Y—C, is further conjugated with an endosomolytic lipid (e.g., fusogenic lipid). Exemplary fusogenic lipids include 1,2-dileoyl-sn-3-phosphoethanolamine (DOPE), phosphatidylethanolamine (POPE), palmitoyloleoylphosphatidylcholine (POPC), (6Z,9Z,28Z,31Z)-heptatriaconta-6,9,28,31-tetraen-19-ol (Di-Lin), N-methyl(2,2-di((9Z,12Z)-octadeca-9,12-dienyl)-1,3-dioxolan-4-yl)methanamine (DLin-k-DMA) and N-methyl-2-(2,2-di((9Z,12Z)-octadeca-9,12-dienyl)-1,3-dioxolan-4-yl)ethanamine (XTC).

In some instances, an endosomolytic moiety is a lipid (e.g., a fusogenic lipid) described in PCT Publication No. WO09/126,933.

### Endosomolytic Small Molecules

In some embodiments, the endosomolytic moiety is a small molecule. In some embodiments, a molecule of Formula (I): A-X—B—Y—C, is further conjugated with an endosomolytic small molecule. Exemplary small molecules suitable as endosomolytic moieties include, but are not limited to, quinine, chloroquine, hydroxychloroquines, amodiaquins (carnoquines), amopyroquines, primaquines, mefloquines, nivaquines, halofantrines, quinone imines, or a combination thereof. In some instances, quinoline endosomolytic moieties include, but are not limited to, 7-chloro-4-(4-diethylamino-1-methylbutyl-amino)quinoline (chloroquine); 7-chloro-4-(4-ethyl-(2-hydroxyethyl)-amino-1-methylbutyl-amino)quinoline (hydroxychloroquine); 7-fluoro-4-(4-diethylamino-1-methylbutyl-amino)quinoline; 4-(4-diethylamino-1-methylbutylamino) quinoline; 7-hydroxy-4-(4-diethyl-amino-1-methylbutylamino)quinoline; 7-chloro-4-(4-diethylamino-1-butylamino)quinoline (desmethylchloroquine); 7-fluoro-4-(4-diethylamino-1-butylamino)quinoline); 4-(4-diethyl-amino-1-butylamino)quinoline; 7-hydroxy-4-(4-diethylamino-1-butylamino)quinoline; 7-chloro-4-(1-carboxy-4-diethylamino-1-butylamino)quinoline; 7-fluoro-4-(1-carboxy-4-diethyl-amino-1-butylamino)quinoline; 4-(1-carboxy-4-diethylamino-1-butylamino) quinoline; 7-hydroxy-4-(1-carboxy-4-diethylamino-1-butylamino)quinoline; 7-chloro-4-(1-carboxy-4-diethylamino-1-methylbutylamino)quinoline; 7-fluoro-4-(1-carboxy-4-diethyl-amino-1-methylbutylamino)quinoline; 4-(1-carboxy-4-diethylamino-1-methylbutylamino)quinoline; 7-hydroxy-4-(1-carboxy-4-diethylamino-1-methylbutylamino)quinoline; 7-fluoro-4-(4-ethyl-(2-hydroxyethyl)-amino-1-methylbutylamino)quinoline; 4-(4-ethyl-(2-hydroxy-ethyl)-amino-1-methylbutylamino-)quinoline; 7-hydroxy-4-(4-ethyl-(2-hydroxyethyl)-amino-1-methylbutylamino)quinoline; hydroxychloroquine phosphate; 7-chloro-4-(4-ethyl-(2-hydroxyethyl-1)-amino-1-butylamino)quinoline (desmethylhydroxychloroquine); 7-fluoro-4-(4-ethyl-(2-hydroxyethyl)-amino-1-butylamino)quinoline; 4-(4-ethyl-(2-hydroxyethyl)-amino-1-butylamino)quinoline; 7-hydroxy-4-(4-ethyl-(2-hydroxyethyl)-amino-1-butylamino) quinoline; 7-chloro-4-(1-carboxy-4-ethyl-(2-hydroxyethyl)-amino-1-butylamino)quinoline; 7-fluoro-4-(1-carboxy-4-ethyl-(2-hydroxyethyl)-amino-1-butylamino)quinoline; 4-(1-carboxy-4-ethyl-(2-hydroxyethyl)-amino-1-butylamino)quinoline; 7-hydroxy-4-(1-carboxy-4-ethyl-(2-hydroxyethyl)-amino-1-butylamino)quinoline; 7-chloro-4-(1-carboxy-4-ethyl-(2-hydroxyethyl)-amino-1-methylbutylamino)quinoline; 7-fluoro-4-(1-carboxy-4-ethyl-(2-hydroxyethyl)-amino-1-methylbutylamino)quinoline; 4-(1-carboxy-4-ethyl-(2-hydroxyethyl)-amino-1-methylbutylamino)quinoline; 7-hydroxy-4-(1-carboxy-4-ethyl-(2-hydroxyethyl)-amino-1-methylbutylamino)quinoline; 8-[(4-aminopentyl)amino-6-methoxydihydrochloride quinoline; 1-acetyl-1,2,3,4-tetrahydroquinoline; 8-[(4-aminopentyl)amino]-6-methoxyquinoline dihydrochloride; 1-butyryl-1,2,3,4-tetrahydroquinoline; 3-chloro-4-(4-hydroxy-alpha,alpha′-bis(2-methyl-1-pyrrolidinyl)-2,5-xylidinoquinoline, 4-[(4-diethyl-amino)-1-methylbutyl-amino]-6-methoxyquinoline; 3-fluoro-4-(4-hydroxy-alpha,alpha′-bis(2-methyl-1-pyrrolidinyl)-2,5-xylidinoquinoline, 4-[(4-diethylamino)-1-methylbutyl-amino]-6-methoxyquinoline; 4-(4-hydroxy-alpha,alpha′-bis(2-methyl-1-pyrrolidinyl)-2,5-xylidinoquinoline; 4-[(4-diethylamino)-1-methylbutyl-amino]-6-methoxyquinoline; 3,4-dihydro-1-(2H)-quinolinecarboxyaldehyde; 1,1′-pentamethylene diquinoleinium diiodide; 8-quinolinol sulfate and amino, aldehyde, carboxylic, hydroxyl, halogen, keto, sulfhydryl and vinyl derivatives or analogs thereof. In some instances, an endosomolytic moiety is a small molecule described in Naisbitt et al (1997, J Pharmacol Exp Therapy 280:884-893) and in U.S. Pat. No. 5,736,557.

### Formula (I) Molecule—Endosomolytic Moiety Conjugates

In some embodiments, one or more endosomolytic moieties are conjugated to a molecule comprising at least one binding moiety, at least one polynucleotide, at least one polymer, or any combinations thereof. In some instances, the endosomolytic moiety is conjugated according to Formula (II):

(A-X—B—Y—C_{c})-L-D Formula II

wherein,

A is a binding moiety;

B is a polynucleotide;

C is a polymer;

X is a bond or a first linker;

Y is a bond or a second linker;

L is a bond or a third linker;

D is an endosomolytic moiety; and

c is an integer between 0 and 1; and

wherein the polynucleotide comprises at least one 5′-vinylphosphonate modified nucleotide; and D is conjugated anywhere on A, B, or C.

In some embodiments, A and C are not attached to B at the same terminus.

In some embodiments, the at least one 2′ modified nucleotide comprises 2′-O-methyl, 2′-O-methoxyethyl (2′-O-MOE), 2′-O-aminopropyl, 2′-deoxy, T-deoxy-2′-fluoro, 2′-O-aminopropyl (2′-O-AP), 2′-O-dimethylaminoethyl (2′-O-DMAOE), 2′-O-dimethylaminopropyl (2′-O-DMAP), T-O-dimethylaminoethyloxyethyl (2′-O-DMAEOE), or 2′-O—N-methylacetamido (2′-O-NMA) modified nucleotide. In some instances, the at least one 2′ modified nucleotide comprises locked nucleic acid (LNA) or ethylene nucleic acid (ENA). In some cases, the at least one modified internucleotide linkage comprises a phosphorothioate linkage or a phosphorodithioate linkage. In some embodiments, the polynucleotide comprises a first polynucleotide and a second polynucleotide hybridized to the first polynucleotide to form a double-stranded polynucleic acid molecule. In some instances, the second polynucleotide comprises at least one modification. In some cases, the first polynucleotide and the second polynucleotide are RNA molecules. In some cases, the first polynucleotide and the second polynucleotide are siRNA molecules. In some embodiments, X, Y, and L are independently a bond or a non-polymeric linker group. In some instances, A is an antibody or binding fragment thereof. In some instances, the antibody or binding fragment thereof comprises a humanized antibody or binding fragment thereof, chimeric antibody or binding fragment thereof, monoclonal antibody or binding fragment thereof, monovalent Fab′, divalent Fab2, single-chain variable fragment (scFv), diabody, minibody, nanobody, single-domain antibody (sdAb), or camelid antibody or binding fragment thereof. In some cases, C is polyethylene glycol.

In some instances, the endosomolytic moiety comprises a polypeptide, a polymer, a lipid, or a small molecule. In some instances, the endosomolytic moiety is an endosomolytic polypeptide. In some cases, the endosomolytic moiety is an endosomolytic polymer. In other cases, the endosomolytic moiety is an endosomolytic lipid. In additional cases, the endosomolytic moiety is an endosomolytic small molecule.

In some instances, the endosomolytic moiety is INF7 or its derivatives thereof. In some cases, the endosomolytic moiety comprises a polypeptide having at least 50%, 55%, 60%, 65%, 70%, 75%, 80%, 85%, 90%, 95%, 96%, 97%, 98%, 99%, or 100% sequence identity to SEQ ID NOs: 1243. In some cases, the endosomolytic moiety comprises SEQ ID NO: 1243. In some cases, the endosomolytic moiety consists of SEQ ID NO: 1243.

In some instances, the endosomolytic moiety is melittin or its derivatives thereof. In some cases, the endosomolytic moiety comprises a polypeptide having at least 50%, 55%, 60%, 65%, 70%, 75%, 80%, 85%, 90%, 95%, 96%, 97%, 98%, 99%, or 100% sequence identity to SEQ ID NOs: 1248. In some cases, the endosomolytic moiety comprises SEQ ID NO: 1248. In some cases, the endosomolytic moiety consists of SEQ ID NO: 1248.

In some instances, the endosomolytic moiety is a sequence as illustrated in Table 10.

In additional cases, the endosomolytic moiety is an endosomolytic polymer, such as for example, a pH-responsive endosomolytic polymer, a membrane-disruptive polymer, a polycation polymer, a polyanion polymer, a pH-responsive membrane-disruptive polymer, or a combination thereof. In additional cases, the endosomolytic moiety comprises a p(alkylacrylic acid) polymer, a p(butyl acrylate-co-methacrylic acid) polymer, a p(styrene-alt-maleic anhydride) polymer, a pyridyldisulfide acrylate (PDSA) polymer, a polymer-PEG conjugate, a polymer-detergent conjugate, or a combination thereof.

In some embodiments, the endosomolytic moiety conjugate is according to Formula (IIa):

D-L-A-X—B—Y—C_{c} Formula IIa

wherein,

A is a binding moiety;

B is a polynucleotide;

C is a polymer;

X is a bond or a first linker;

Y is a bond or a second linker;

L is a bond or a third linker;

D is an endosomolytic moiety; and

c is an integer of 1; and

wherein the polynucleotide comprises at least one 5′-vinylphosphonate modified nucleotide.

In some embodiments, A and C are not attached to B at the same terminus.

In some embodiments, the at least one 2′ modified nucleotide comprises 2′-O-methyl, 2′-O-methoxyethyl (2′-O-MOE), 2′-O-aminopropyl, 2′-deoxy, T-deoxy-2′-fluoro, 2′-O-aminopropyl (2′-O-AP), 2′-O-dimethylaminoethyl (2′-O-DMAOE), 2′-O-dimethylaminopropyl (2′-O-DMAP), T-O-dimethylaminoethyloxyethyl (2′-O-DMAEOE), or 2′-O—N-methylacetamido (2′-O-NMA) modified nucleotide. In some instances, the at least one 2′ modified nucleotide comprises locked nucleic acid (LNA) or ethylene nucleic acid (ENA). In some cases, the at least one modified internucleotide linkage comprises a phosphorothioate linkage or a phosphorodithioate linkage. In some embodiments, the polynucleotide comprises a first polynucleotide and a second polynucleotide hybridized to the first polynucleotide to form a double-stranded polynucleic acid molecule. In some instances, the second polynucleotide comprises at least one modification. In some cases, the first polynucleotide and the second polynucleotide are RNA molecules. In some cases, the first polynucleotide and the second polynucleotide are siRNA molecules. In some embodiments, X, Y, and L are independently a bond or a non-polymeric linker group. In some instances, A is an antibody or binding fragment thereof. In some instances, the antibody or binding fragment thereof comprises a humanized antibody or binding fragment thereof, chimeric antibody or binding fragment thereof, monoclonal antibody or binding fragment thereof, monovalent Fab′, divalent Fab2, single-chain variable fragment (scFv), diabody, minibody, nanobody, single-domain antibody (sdAb), or camelid antibody or binding fragment thereof. In some cases, C is polyethylene glycol.

In some instances, the endosomolytic moiety comprises a polypeptide, a polymer, a lipid, or a small molecule. In some instances, the endosomolytic moiety is an endosomolytic polypeptide. In some cases, the endosomolytic moiety is an endosomolytic polymer. In other cases, the endosomolytic moiety is an endosomolytic lipid. In additional cases, the endosomolytic moiety is an endosomolytic small molecule.

In some instances, the endosomolytic moiety is INF7 or its derivatives thereof. In some cases, the endosomolytic moiety comprises a polypeptide having at least 50%, 55%, 60%, 65%, 70%, 75%, 80%, 85%, 90%, 95%, 96%, 97%, 98%, 99%, or 100% sequence identity to SEQ ID NOs: 1243. In some cases, the endosomolytic moiety comprises SEQ ID NO: 1243. In some cases, the endosomolytic moiety consists of SEQ ID NO: 1243.

In some instances, the endosomolytic moiety is melittin or its derivatives thereof. In some cases, the endosomolytic moiety comprises a polypeptide having at least 50%, 55%, 60%, 65%, 70%, 75%, 80%, 85%, 90%, 95%, 96%, 97%, 98%, 99%, or 100% sequence identity to SEQ ID NOs: 1248. In some cases, the endosomolytic moiety comprises SEQ ID NO: 1248. In some cases, the endosomolytic moiety consists of SEQ ID NO: 1248.

In some instances, the endosomolytic moiety is a sequence as illustrated in Table 10.

In additional cases, the endosomolytic moiety is an endosomolytic polymer, such as for example, a pH-responsive endosomolytic polymer, a membrane-disruptive polymer, a polycation polymer, a polyanion polymer, a pH-responsive membrane-disruptive polymer, or a combination thereof. In additional cases, the endosomolytic moiety comprises a p(alkylacrylic acid) polymer, a p(butyl acrylate-co-methacrylic acid) polymer, a p(styrene-alt-maleic anhydride) polymer, a pyridyldisulfide acrylate (PDSA) polymer, a polymer-PEG conjugate, a polymer-detergent conjugate, or a combination thereof.

In some instances, the endosomolytic moiety conjugate is according to Formula (IIb):

A-X—B-L-D Formula IIb

wherein,

A is a binding moiety;

B is a polynucleotide;

X is a bond or a first linker;

L is a bond or a third linker; and

D is an endosomolytic moiety; and

wherein the polynucleotide comprises at least one 5′-vinylphosphonate modified nucleotide.

In some embodiments, A and C are not attached to B at the same terminus.

In some embodiments, the at least one 2′ modified nucleotide comprises 2′-O-methyl, 2′-O-methoxyethyl (2′-O-MOE), 2′-O-aminopropyl, 2′-deoxy, T-deoxy-2′-fluoro, 2′-O-aminopropyl (2′-O-AP), 2′-O-dimethylaminoethyl (2′-O-DMAOE), 2′-O-dimethylaminopropyl (2′-O-DMAP), T-O-dimethylaminoethyloxyethyl (2′-O-DMAEOE), or 2′-O—N-methylacetamido (2′-O-NMA) modified nucleotide. In some instances, the at least one 2′ modified nucleotide comprises locked nucleic acid (LNA) or ethylene nucleic acid (ENA). In some cases, the at least one modified internucleotide linkage comprises a phosphorothioate linkage or a phosphorodithioate linkage. In some embodiments, the polynucleotide comprises a first polynucleotide and a second polynucleotide hybridized to the first polynucleotide to form a double-stranded polynucleic acid molecule. In some instances, the second polynucleotide comprises at least one modification. In some cases, the first polynucleotide and the second polynucleotide are RNA molecules. In some cases, the first polynucleotide and the second polynucleotide are siRNA molecules. In some embodiments, X and L are independently a bond or a non-polymeric linker group. In some instances, A is an antibody or binding fragment thereof. In some instances, the antibody or binding fragment thereof comprises a humanized antibody or binding fragment thereof, chimeric antibody or binding fragment thereof, monoclonal antibody or binding fragment thereof, monovalent Fab′, divalent Fab2, single-chain variable fragment (scFv), diabody, minibody, nanobody, single-domain antibody (sdAb), or camelid antibody or binding fragment thereof. In some cases, C is polyethylene glycol.

In some instances, the endosomolytic moiety comprises a polypeptide, a polymer, a lipid, or a small molecule. In some instances, the endosomolytic moiety is an endosomolytic polypeptide. In some cases, the endosomolytic moiety is an endosomolytic polymer. In other cases, the endosomolytic moiety is an endosomolytic lipid. In additional cases, the endosomolytic moiety is an endosomolytic small molecule.

In some instances, the endosomolytic moiety is INF7 or its derivatives thereof. In some cases, the endosomolytic moiety comprises a polypeptide having at least 50%, 55%, 60%, 65%, 70%, 75%, 80%, 85%, 90%, 95%, 96%, 97%, 98%, 99%, or 100% sequence identity to SEQ ID NOs: 1243. In some cases, the endosomolytic moiety comprises SEQ ID NO: 1243. In some cases, the endosomolytic moiety consists of SEQ ID NO: 1243.

In some instances, the endosomolytic moiety is melittin or its derivatives thereof. In some cases, the endosomolytic moiety comprises a polypeptide having at least 50%, 55%, 60%, 65%, 70%, 75%, 80%, 85%, 90%, 95%, 96%, 97%, 98%, 99%, or 100% sequence identity to SEQ ID NOs: 1248. In some cases, the endosomolytic moiety comprises SEQ ID NO: 1248. In some cases, the endosomolytic moiety consists of SEQ ID NO: 1248.

In some instances, the endosomolytic moiety is a sequence as illustrated in Table 10.

In additional cases, the endosomolytic moiety is an endosomolytic polymer, such as for example, a pH-responsive endosomolytic polymer, a membrane-disruptive polymer, a polycation polymer, a polyanion polymer, a pH-responsive membrane-disruptive polymer, or a combination thereof. In additional cases, the endosomolytic moiety comprises a p(alkylacrylic acid) polymer, a p(butyl acrylate-co-methacrylic acid) polymer, a p(styrene-alt-maleic anhydride) polymer, a pyridyldisulfide acrylate (PDSA) polymer, a polymer-PEG conjugate, a polymer-detergent conjugate, or a combination thereof.

In some instances, the endosomolytic moiety conjugate is according to Formula (IIc):

A-X—B—Y—C_{c}-L-D Formula IIc

wherein,

A is a binding moiety;

B is a polynucleotide;

C is a polymer;

X is a bond or a first linker;

Y is a bond or a second linker;

L is a bond or a third linker;

D is an endosomolytic moiety; and

c is an integer of 1; and

wherein the polynucleotide comprises at least one 5′-vinylphosphonate modified nucleotide.

In some embodiments, A and C are not attached to B at the same terminus.

In some embodiments, the at least one 2′ modified nucleotide comprises 2′-O-methyl, 2′-O-methoxyethyl (2′-O-MOE), 2′-O-aminopropyl, 2′-deoxy, T-deoxy-2′-fluoro, 2′-O-aminopropyl (2′-O-AP), 2′-O-dimethylaminoethyl (2′-O-DMAOE), 2′-O-dimethylaminopropyl (2′-O-DMAP), T-O-dimethylaminoethyloxyethyl (2′-O-DMAEOE), or 2′-O—N-methylacetamido (2′-O-NMA) modified nucleotide. In some instances, the at least one 2′ modified nucleotide comprises locked nucleic acid (LNA) or ethylene nucleic acid (ENA). In some cases, the at least one modified internucleotide linkage comprises a phosphorothioate linkage or a phosphorodithioate linkage. In some embodiments, the polynucleotide comprises a first polynucleotide and a second polynucleotide hybridized to the first polynucleotide to form a double-stranded polynucleic acid molecule. In some instances, the second polynucleotide comprises at least one modification. In some cases, the first polynucleotide and the second polynucleotide are RNA molecules. In some cases, the first polynucleotide and the second polynucleotide are siRNA molecules. In some embodiments, X, Y, and L are independently a bond or a non-polymeric linker group. In some instances, A is an antibody or binding fragment thereof. In some instances, the antibody or binding fragment thereof comprises a humanized antibody or binding fragment thereof, chimeric antibody or binding fragment thereof, monoclonal antibody or binding fragment thereof, monovalent Fab′, divalent Fab2, single-chain variable fragment (scFv), diabody, minibody, nanobody, single-domain antibody (sdAb), or camelid antibody or binding fragment thereof. In some cases, C is polyethylene glycol.

In some instances, the endosomolytic moiety comprises a polypeptide, a polymer, a lipid, or a small molecule. In some instances, the endosomolytic moiety is an endosomolytic polypeptide. In some cases, the endosomolytic moiety is an endosomolytic polymer. In other cases, the endosomolytic moiety is an endosomolytic lipid. In additional cases, the endosomolytic moiety is an endosomolytic small molecule.

In some instances, the endosomolytic moiety is INF7 or its derivatives thereof. In some cases, the endosomolytic moiety comprises a polypeptide having at least 50%, 55%, 60%, 65%, 70%, 75%, 80%, 85%, 90%, 95%, 96%, 97%, 98%, 99%, or 100% sequence identity to SEQ ID NOs: 1243. In some cases, the endosomolytic moiety comprises SEQ ID NO: 1243. In some cases, the endosomolytic moiety consists of SEQ ID NO: 1243.

In some instances, the endosomolytic moiety is melittin or its derivatives thereof. In some cases, the endosomolytic moiety comprises a polypeptide having at least 50%, 55%, 60%, 65%, 70%, 75%, 80%, 85%, 90%, 95%, 96%, 97%, 98%, 99%, or 100% sequence identity to SEQ ID NOs: 1248. In some cases, the endosomolytic moiety comprises SEQ ID NO: 1248. In some cases, the endosomolytic moiety consists of SEQ ID NO: 1248.

In some instances, the endosomolytic moiety is a sequence as illustrated in Table 10.

In additional cases, the endosomolytic moiety is an endosomolytic polymer, such as for example, a pH-responsive endosomolytic polymer, a membrane-disruptive polymer, a polycation polymer, a polyanion polymer, a pH-responsive membrane-disruptive polymer, or a combination thereof. In additional cases, the endosomolytic moiety comprises a p(alkylacrylic acid) polymer, a p(butyl acrylate-co-methacrylic acid) polymer, a p(styrene-alt-maleic anhydride) polymer, a pyridyldisulfide acrylate (PDSA) polymer, a polymer-PEG conjugate, a polymer-detergent conjugate, or a combination thereof.

In some instances, the endosomolytic moiety conjugate is according to Formula (IId):

A-L-D-X—B—Y—C_{c} Formula IId

wherein,

A is a binding moiety;

B is a polynucleotide;

C is a polymer;

X is a bond or a first linker;

Y is a bond or a second linker;

L is a bond or a third linker;

D is an endosomolytic moiety; and

c is an integer of 1; and

wherein the polynucleotide comprises at least one 5′-vinylphosphonate modified nucleotide.

In some embodiments, A and C are not attached to B at the same terminus.

In some embodiments, the at least one 2′ modified nucleotide comprises 2′-O-methyl, 2′-O-methoxyethyl (2′-O-MOE), 2′-O-aminopropyl, 2′-deoxy, T-deoxy-2′-fluoro, 2′-O-aminopropyl (2′-O-AP), 2′-O-dimethylaminoethyl (2′-O-DMAOE), 2′-O-dimethylaminopropyl (2′-O-DMAP), T-O-dimethylaminoethyloxyethyl (2′-O-DMAEOE), or 2′-O—N-methylacetamido (2′-O-NMA) modified nucleotide. In some instances, the at least one 2′ modified nucleotide comprises locked nucleic acid (LNA) or ethylene nucleic acid (ENA). In some cases, the at least one modified internucleotide linkage comprises a phosphorothioate linkage or a phosphorodithioate linkage. In some embodiments, the polynucleotide comprises a first polynucleotide and a second polynucleotide hybridized to the first polynucleotide to form a double-stranded polynucleic acid molecule. In some instances, the second polynucleotide comprises at least one modification. In some cases, the first polynucleotide and the second polynucleotide are RNA molecules. In some cases, the first polynucleotide and the second polynucleotide are siRNA molecules. In some embodiments, X, Y, and L are independently a bond or a non-polymeric linker group. In some instances, A is an antibody or binding fragment thereof. In some instances, the antibody or binding fragment thereof comprises a humanized antibody or binding fragment thereof, chimeric antibody or binding fragment thereof, monoclonal antibody or binding fragment thereof, monovalent Fab′, divalent Fab2, single-chain variable fragment (scFv), diabody, minibody, nanobody, single-domain antibody (sdAb), or camelid antibody or binding fragment thereof. In some cases, C is polyethylene glycol.

In some instances, the endosomolytic moiety comprises a polypeptide, a polymer, a lipid, or a small molecule. In some instances, the endosomolytic moiety is an endosomolytic polypeptide. In some cases, the endosomolytic moiety is an endosomolytic polymer. In other cases, the endosomolytic moiety is an endosomolytic lipid. In additional cases, the endosomolytic moiety is an endosomolytic small molecule.

In some instances, the endosomolytic moiety is INF7 or its derivatives thereof. In some cases, the endosomolytic moiety comprises a polypeptide having at least 50%, 55%, 60%, 65%, 70%, 75%, 80%, 85%, 90%, 95%, 96%, 97%, 98%, 99%, or 100% sequence identity to SEQ ID NOs: 1243. In some cases, the endosomolytic moiety comprises SEQ ID NO: 1243. In some cases, the endosomolytic moiety consists of SEQ ID NO: 1243.

In some instances, the endosomolytic moiety is melittin or its derivatives thereof. In some cases, the endosomolytic moiety comprises a polypeptide having at least 50%, 55%, 60%, 65%, 70%, 75%, 80%, 85%, 90%, 95%, 96%, 97%, 98%, 99%, or 100% sequence identity to SEQ ID NOs: 1248. In some cases, the endosomolytic moiety comprises SEQ ID NO: 1248. In some cases, the endosomolytic moiety consists of SEQ ID NO: 1248.

In some instances, the endosomolytic moiety is a sequence as illustrated in Table 10.

In additional cases, the endosomolytic moiety is an endosomolytic polymer, such as for example, a pH-responsive endosomolytic polymer, a membrane-disruptive polymer, a polycation polymer, a polyanion polymer, a pH-responsive membrane-disruptive polymer, or a combination thereof. In additional cases, the endosomolytic moiety comprises a p(alkylacrylic acid) polymer, a p(butyl acrylate-co-methacrylic acid) polymer, a p(styrene-alt-maleic anhydride) polymer, a pyridyldisulfide acrylate (PDSA) polymer, a polymer-PEG conjugate, a polymer-detergent conjugate, or a combination thereof.

### Linkers

In some embodiments, a linker described herein is a cleavable linker or a non-cleavable linker. In some instances, the linker is a cleavable linker. In some instances, the linker is an acid cleavable linker. In some instances, the linker is a non-cleavable linker. In some instances, the linker includes a C₁-C₆ alkyl group (e.g., a C₅, C₄, C₃, C₂, or C₁ alkyl group). In some instances, the linker includes homobifunctional cross linkers, heterobifunctional cross linkers, and the like. In some instances, the liker is a traceless linker (or a zero-length linker). In some instances, the linker is a non-polymeric linker. In some cases, the linker is a non-peptide linker or a linker that does not contain an amino acid residue.

In some instances, the linker comprises a homobifuctional linker. Exemplary homobifuctional linkers include, but are not limited to, Lomant's reagent dithiobis (succinimidylpropionate) DSP, 3′3′-dithiobis(sulfosuccinimidyl proprionate (DTSSP), disuccinimidyl suberate (DSS), bis(sulfosuccinimidyl)suberate (BS), disuccinimidyl tartrate (DST), disulfosuccinimidyl tartrate (sulfo DST), ethylene glycobis(succinimidylsuccinate) (EGS), disuccinimidyl glutarate (DSG), N,N′-disuccinimidyl carbonate (DSC), dimethyl adipimidate (DMA), dimethyl pimelimidate (DMP), dimethyl suberimidate (DMS), dimethyl-3,3′-dithiobispropionimidate (DTBP), 1,4-di-3′-(2′-pyridyldithio)propionamido)butane (DPDPB), bismaleimidohexane (BMH), aryl halide-containing compound (DFDNB), such as e.g. 1,5-difluoro-2,4-dinitrobenzene or 1,3-difluoro-4,6-dinitrobenzene, 4,4′-difluoro-3,3′-dinitrophenylsulfone (DFDNPS), bis-[β-(4-azidosalicylamido)ethyl]disulfide (BASED), formaldehyde, glutaraldehyde, 1,4-butanediol diglycidyl ether, adipic acid dihydrazide, carbohydrazide, o-toluidine, 3,3′-dimethylbenzidine, benzidine, α,α′-p-diaminodiphenyl, diiodo-p-xylene sulfonic acid, N,N′-ethylene-bis(iodoacetamide), or N,N′-hexamethylene-bis(iodoacetamide).

In some embodiments, the linker comprises a heterobifunctional linker. Exemplary heterobifunctional linker include, but are not limited to, amine-reactive and sulfhydryl cross-linkers such as N-succinimidyl 3-(2-pyridyldithio)propionate (sPDP), long-chain N-succinimidyl 3-(2-pyridyldithio)propionate (LC-sPDP), water-soluble-long-chain N-succinimidyl 3-(2-pyridyldithio) propionate (sulfo-LC-sPDP), succinimidyloxycarbonyl-α-methyl-α-(2-pyridyldithio)toluene (sMPT), sulfosuccinimidyl-6-[α-methyl-α-(2-pyridyldithio)toluamido]hexanoate (sulfo-LC-sMPT), succinimidyl-4-(N-maleimidomethyl)cyclohexane-1-carboxylate (sMCC), sulfosuccinimidyl-4-(N-maleimidomethyl)cyclohexane-1-carboxylate (sulfo-sMCC), m-maleimidobenzoyl-N-hydroxysuccinimide ester (MBs), m-maleimidobenzoyl-N-hydroxysulfosuccinimide ester (sulfo-MBs), N-succinimidyl(4-iodoacteyl)aminobenzoate (sIAB), sulfosuccinimidyl(4-iodoacteyl)aminobenzoate (sulfo-sIAB), succinimidyl-4-(p-maleimidophenyl)butyrate (sMPB), sulfosuccinimidyl-4-(p-maleimidophenyl)butyrate (sulfo-sMPB), N—(γ-maleimidobutyryloxy)succinimide ester (GMBs), N—(γ-maleimidobutyryloxy)sulfosuccinimide ester (sulfo-GMBs), succinimidyl 6-((iodoacetyl)amino)hexanoate (sIAX), succinimidyl 6-[6-(((iodoacetyl)amino)hexanoyl)amino]hexanoate (sIAXX), succinimidyl 4-(((iodoacetyl)amino)methyl)cyclohexane-1-carboxylate (sIAC), succinimidyl 6-((((4-iodoacetyl)amino)methyl)cyclohexane-1-carbonyl)amino) hexanoate (sIACX), p-nitrophenyl iodoacetate (NPIA), carbonyl-reactive and sulfhydryl-reactive cross-linkers such as 4-(4-N-maleimidophenyl)butyric acid hydrazide (MPBH), 4-(N-maleimidomethyl)cyclohexane-1-carboxyl-hydrazide-8 (M₂C₂H), 3-(2-pyridyldithio)propionyl hydrazide (PDPH), amine-reactive and photoreactive cross-linkers such as N-hydroxysuccinimidyl-4-azidosalicylic acid (NHs-AsA), N-hydroxysulfosuccinimidyl-4-azidosalicylic acid (sulfo-NHs-AsA), sulfosuccinimidyl-(4-azidosalicylamido)hexanoate (sulfo-NHs-LC-AsA), sulfosuccinimidyl-2-(ρ-azidosalicylamido)ethyl-1,3′-dithiopropionate (sAsD), N-hydroxysuccinimidyl-4-azidobenzoate (HsAB), N-hydroxysulfosuccinimidyl-4-azidobenzoate (sulfo-HsAB), N-succinimidyl-6-(4′-azido-2′-nitrophenylamino)hexanoate (sANPAH), sulfosuccinimidyl-6-(4′-azido-2′-nitrophenylamino)hexanoate (sulfo-sANPAH), N-5-azido-2-nitrobenzoyloxysuccinimide (ANB-NOs), sulfosuccinimidyl-2-(m-azido-o-nitrobenzamido)-ethyl-1,3′-dithiopropionate (sAND), N-succinimidyl-4(4-azidophenyl)1,3′-dithiopropionate (sADP), N-sulfosuccinimidyl(4-azidophenyl)-1,3′-dithiopropionate (sulfo-sADP), sulfosuccinimidyl 4-(p-azidophenyl)butyrate (sulfo-sAPB), sulfosuccinimidyl 2-(7-azido-4-methylcoumarin-3-acetamide)ethyl-1,3′-dithiopropionate (sAED), sulfosuccinimidyl 7-azido-4-methylcoumain-3-acetate (sulfo-sAMCA), p-nitrophenyl diazopyruvate (pNPDP), ρ-nitrophenyl-2-diazo-3,3,3-trifluoropropionate (PNP-DTP), sulfhydryl-reactive and photoreactive cross-linkers such as 1-(ρ-Azidosalicylamido)-4-(iodoacetamido)butane (AsIB), N-[4-(ρ-azidosalicylamido)butyl]-3′-(2′-pyridyldithio)propionamide (APDP), benzophenone-4-iodoacetamide, benzophenone-4-maleimide carbonyl-reactive and photoreactive cross-linkers such as ρ-azidobenzoyl hydrazide (ABH), carboxylate-reactive and photoreactive cross-linkers such as 4-(ρ-azidosalicylamido)butylamine (AsBA), and arginine-reactive and photoreactive cross-linkers such as ρ-azidophenyl glyoxal (APG).

In some instances, the linker comprises a reactive functional group. In some cases, the reactive functional group comprises a nucleophilic group that is reactive to an electrophilic group present on a binding moiety. Exemplary electrophilic groups include carbonyl groups—such as aldehyde, ketone, carboxylic acid, ester, amide, enone, acyl halide or acid anhydride. In some embodiments, the reactive functional group is aldehyde. Exemplary nucleophilic groups include hydrazide, oxime, amino, hydrazine, thiosemicarbazone, hydrazine carboxylate, and arylhydrazide.

In some embodiments, the linker comprises a maleimide goup. In some instances, the maleimide group is also referred to as a maleimide spacer. In some instances, the maleimide group further encompasses a caproic acid, forming maleimidocaproyl (me). In some cases, the linker comprises maleimidocaproyl (me). In some cases, the linker is maleimidocaproyl (me). In other instances, the maleimide group comprises a maleimidomethyl group, such as succinimidyl-4-(N-maleimidomethyl)cyclohexane-1-carboxylate (sMCC) or sulfosuccinimidyl-4-(N-maleimidomethyl)cyclohexane-1-carboxylate (sulfo-sMCC) described above.

In some embodiments, the maleimide group is a self-stabilizing maleimide. In some instances, the self-stabilizing maleimide utilizes diaminopropionic acid (DPR) to incorporate a basic amino group adjacent to the maleimide to provide intramolecular catalysis of tiosuccinimide ring hydrolysis, thereby eliminating maleimide from undergoing an elimination reaction through a retro-Michael reaction. In some instances, the self-stabilizing maleimide is a maleimide group described in Lyon, et al., “Self-hydrolyzing maleimides improve the stability and pharmacological properties of antibody-drug conjugates,” Nat. Biotechnol. 32(10):1059-1062 (2014). In some instances, the linker comprises a self-stabilizing maleimide. In some instances, the linker is a self-stabilizing maleimide.

In some embodiments, the linker comprises a peptide moiety. In some instances, the peptide moiety comprises at least 2, 3, 4, 5, 6, 7, 8, or more amino acid residues. In some instances, the peptide moiety is a cleavable peptide moiety (e.g., either enzymatically or chemically). In some instances, the peptide moiety is a non-cleavable peptide moiety. In some instances, the peptide moiety comprises Val-Cit (valine-citrulline), Gly-Gly-Phe-Gly (SEQ ID NO: 1270), Phe-Lys, Val-Lys, Gly-Phe-Lys, Phe-Phe-Lys, Ala-Lys, Val-Arg, Phe-Cit, Phe-Arg, Leu-Cit, Ile-Cit, Trp-Cit, Phe-Ala, Ala-Leu-Ala-Leu (SEQ ID NO: 1271), or Gly-Phe-Leu-Gly (SEQ ID NO: 1272). In some instances, the linker comprises a peptide moiety such as: Val-Cit (valine-citrulline), Gly-Gly-Phe-Gly (SEQ ID NO: 1270), Phe-Lys, Val-Lys, Gly-Phe-Lys, Phe-Phe-Lys, Ala-Lys, Val-Arg, Phe-Cit, Phe-Arg, Leu-Cit, Ile-Cit, Trp-Cit, Phe-Ala, Ala-Leu-Ala-Leu (SEQ ID NO: 1271), or Gly-Phe-Leu-Gly (SEQ ID NO: 1272). In some cases, the linker comprises Val-Cit. In some cases, the linker is Val-Cit.

In some embodiments, the linker comprises a benzoic acid group, or its derivatives thereof. In some instances, the benzoic acid group or its derivatives thereof comprise paraaminobenzoic acid (PABA). In some instances, the benzoic acid group or its derivatives thereof comprise gamma-aminobutyric acid (GABA).

In some embodiments, the linker comprises one or more of a maleimide group, a peptide moiety, and/or a benzoic acid group, in any combination. In some embodiments, the linker comprises a combination of a maleimide group, a peptide moiety, and/or a benzoic acid group. In some instances, the maleimide group is maleimidocaproyl (me). In some instances, the peptide group is val-cit. In some instances, the benzoic acid group is PABA. In some instances, the linker comprises a mc-val-cit group. In some cases, the linker comprises a val-cit-PABA group. In additional cases, the linker comprises a mc-val-cit-PABA group.

In some embodiments, the linker is a self-immolative linker or a self-elimination linker. In some cases, the linker is a self-immolative linker. In other cases, the linker is a self-elimination linker (e.g., a cyclization self-elimination linker). In some instances, the linker comprises a linker described in U.S. Pat. No. 9,089,614 or PCT Publication No. WO2015038426.

In some embodiments, the linker is a dendritic type linker. In some instances, the dendritic type linker comprises a branching, multifunctional linker moiety. In some instances, the dendritic type linker is used to increase the molar ratio of polynucleotide B to the binding moiety A. In some instances, the dendritic type linker comprises PAMAM dendrimers.

In some embodiments, the linker is a traceless linker or a linker in which after cleavage does not leave behind a linker moiety (e.g., an atom or a linker group) to a binding moiety A, a polynucleotide B, a polymer C, or an endosomolytic moiety D. Exemplary traceless linkers include, but are not limited to, germanium linkers, silicium linkers, sulfur linkers, selenium linkers, nitrogen linkers, phosphorus linkers, boron linkers, chromium linkers, or phenylhydrazide linker. In some cases, the linker is a traceless aryl-triazene linker as described in Hejesen, et al., “A traceless aryl-triazene linker for DNA-directed chemistry,” Org Biomol Chem 11(15): 2493-2497 (2013). In some instances, the linker is a traceless linker described in Blaney, et al., “Traceless solid-phase organic synthesis,” Chem. Rev. 102: 2607-2024 (2002). In some instances, a linker is a traceless linker as described in U.S. Pat. No. 6,821,783.

In some instances, the linker comprises a functional group that exerts steric hinderance at the site of bonding between the linker and a conjugating moiety (e.g., A, B, C, or D described herein). In some instances, the steric hinderance is a steric hindrance around a disulfide bond. Exemplary linkers that exhibit steric hinderance comprises a heterobifuctional linker, such as a heterobifuctional linker described above. In some cases, a linker that exhibits steric hinderance comprises SMCC and SPDB.

In some instances, the linker is an acid cleavable linker. In some instances, the acid cleavable linker comprises a hydrazone linkage, which is susceptible to hydrolytic cleavage. In some cases, the acid cleavable linker comprises a thiomaleamic acid linker. In some cases, the acid cleavable linker is a thiomaleamic acid linker as described in Castaneda, et al, “Acid-cleavable thiomaleamic acid linker for homogeneous antibody-drug conjugation,” Chem. Commun. 49: 8187-8189 (2013).

In some instances, the linker is a linker described in U.S. Pat. Nos. 6,884,869; 7,498,298; 8,288,352; 8,609,105; or 8,697,688; U.S. Patent Publication Nos. 2014/0127239; 2013/028919; 2014/286970; 2013/0309256; 2015/037360; or 2014/0294851; or PCT Publication Nos. WO2015057699; WO2014080251; WO2014197854; WO2014145090; or WO2014177042.

In some embodiments, X, Y, and L are independently a bond or a linker. In some instances, X, Y, and L are independently a bond. In some cases, X, Y, and L are independently a linker.

In some instances, X is a bond or a linker. In some instances, X is a bond. In some instances, X is a linker. In some instances, the linker is a C₁-C₆ alkyl group. In some cases, X is a C₁-C₆ alkyl group, such as for example, a C₅, C₄, C₃, C₂, or C₁ alkyl group. In some cases, the C₁-C₆ alkyl group is an unsubstituted C₁-C₆ alkyl group. As used in the context of a linker, and in particular in the context of X, alkyl means a saturated straight or branched hydrocarbon radical containing up to six carbon atoms. In some instances, X is a non-polymeric linker. In some instances, X includes a homobifuctional linker or a heterobifuctional linker described supra. In some cases, X includes a heterobifunctional linker. In some cases, X includes sMCC. In other instances, X includes a heterobifuctional linker optionally conjugated to a C₁-C₆ alkyl group. In other instances, X includes sMCC optionally conjugated to a C₁-C₆ alkyl group. In additional instances, X does not include a homobifuctional linker or a heterobifunctional linker described supra.

In some instances, Y is a bond or a linker. In some instances, Y is a bond. In other cases, Y is a linker. In some embodiments, Y is a C₁-C₆ alkyl group. In some instances, Y is a homobifuctional linker or a heterobifunctional linker described supra. In some instances, Y is a homobifuctional linker described supra. In some instances, Y is a heterobifunctional linker described supra. In some instances, Y comprises a maleimide group, such as maleimidocaproyl (me) or a self-stabilizing maleimide group described above. In some instances, Y comprises a peptide moiety, such as Val-Cit. In some instances, Y comprises a benzoic acid group, such as PABA. In additional instances, Y comprises a combination of a maleimide group, a peptide moiety, and/or a benzoic acid group. In additional instances, Y comprises a me group. In additional instances, Y comprises a mc-val-cit group. In additional instances, Y comprises a val-cit-PABA group. In additional instances, Y comprises a mc-val-cit-PABA group.

In some instances, L is a bond or a linker. In some cases, L is a bond. In other cases, L is a linker. In some embodiments, L is a C₁-C₆ alkyl group. In some instances, L is a homobifuctional linker or a heterobifunctional linker described supra. In some instances, L is a homobifuctional linker described supra. In some instances, L is a heterobifunctional linker described supra. In some instances, L comprises a maleimide group, such as maleimidocaproyl (me) or a self-stabilizing maleimide group described above. In some instances, L comprises a peptide moiety, such as Val-Cit. In some instances, L comprises a benzoic acid group, such as PABA. In additional instances, L comprises a combination of a maleimide group, a peptide moiety, and/or a benzoic acid group. In additional instances, L comprises a me group. In additional instances, L comprises a mc-val-cit group. In additional instances, L comprises a val-cit-PABA group. In additional instances, L comprises a mc-val-cit-PABA group.

### Methods of Use

In some embodiments, a composition or a pharmaceutical formulation described herein comprising a binding moiety conjugated to a polynucleic acid molecule and a polymer is used for the treatment of a disease or disorder. In some instances, the disease or disorder is a cancer. In some embodiments, a composition or a pharmaceutical formulation described herein is used as an immunotherapy for the treatment of a disease or disorder. In some instances, the immunotherapy is an immuno-oncology therapy.

### Cancer

In some embodiments, a composition or a pharmaceutical formulation described herein is used for the treatment of cancer. In some instances, the cancer is a solid tumor. In some instances, the cancer is a hematologic malignancy. In some instances, the cancer is a relapsed or refractory cancer, or a metastatic cancer. In some instances, the solid tumor is a relapsed or refractory solid tumor, or a metastatic solid tumor. In some cases, the hematologic malignancy is a relapsed or refractory hematologic malignancy, or a metastatic hematologic malignancy.

In some embodiments, the cancer is a solid tumor. Exemplary solid tumor includes, but is not limited to, anal cancer, appendix cancer, bile duct cancer (i.e., cholangiocarcinoma), bladder cancer, brain tumor, breast cancer, cervical cancer, colon cancer, cancer of Unknown Primary (CUP), esophageal cancer, eye cancer, fallopian tube cancer, gastroenterological cancer, kidney cancer, liver cancer, lung cancer, medulloblastoma, melanoma, oral cancer, ovarian cancer, pancreatic cancer, parathyroid disease, penile cancer, pituitary tumor, prostate cancer, rectal cancer, skin cancer, stomach cancer, testicular cancer, throat cancer, thyroid cancer, uterine cancer, vaginal cancer, or vulvar cancer.

In some instances, a composition or a pharmaceutical formulation described herein comprising a binding moiety conjugated to a polynucleic acid molecule and a polymer is used for the treatment of a solid tumor. In some instances, a composition or a pharmaceutical formulation described herein comprising a binding moiety conjugated to a polynucleic acid molecule and a polymer is used for the treatment of anal cancer, appendix cancer, bile duct cancer (i.e., cholangiocarcinoma), bladder cancer, brain tumor, breast cancer, cervical cancer, colon cancer, cancer of Unknown Primary (CUP), esophageal cancer, eye cancer, fallopian tube cancer, gastroenterological cancer, kidney cancer, liver cancer, lung cancer, medulloblastoma, melanoma, oral cancer, ovarian cancer, pancreatic cancer, parathyroid disease, penile cancer, pituitary tumor, prostate cancer, rectal cancer, skin cancer, stomach cancer, testicular cancer, throat cancer, thyroid cancer, uterine cancer, vaginal cancer, or vulvar cancer. In some instances, the solid tumor is a relapsed or refractory solid tumor, or a metastatic solid tumor.

In some instances, the cancer is a hematologic malignancy. In some instances, the hematologic malignancy is a leukemia, a lymphoma, a myeloma, a non-Hodgkin's lymphoma, or a Hodgkin's lymphoma. In some instances, the hematologic malignancy comprises chronic lymphocytic leukemia (CLL), small lymphocytic lymphoma (SLL), high risk CLL, a non-CLL/SLL lymphoma, prolymphocytic leukemia (PLL), follicular lymphoma (FL), diffuse large B-cell lymphoma (DLBCL), mantle cell lymphoma (MCL), Waldenstrom's macroglobulinemia, multiple myeloma, extranodal marginal zone B cell lymphoma, nodal marginal zone B cell lymphoma, Burkitt's lymphoma, non-Burkitt high grade B cell lymphoma, primary mediastinal B-cell lymphoma (PMBL), immunoblastic large cell lymphoma, precursor B-lymphoblastic lymphoma, B cell prolymphocytic leukemia, lymphoplasmacytic lymphoma, splenic marginal zone lymphoma, plasma cell myeloma, plasmacytoma, mediastinal (thymic) large B cell lymphoma, intravascular large B cell lymphoma, primary effusion lymphoma, or lymphomatoid granulomatosis.

In some instances, a composition or a pharmaceutical formulation described herein comprising a binding moiety conjugated to a polynucleic acid molecule and a polymer is used for the treatment of a hematologic malignancy. In some instances, a composition or a pharmaceutical formulation described herein comprising a binding moiety conjugated to a polynucleic acid molecule and a polymer is used for the treatment of a leukemia, a lymphoma, a myeloma, a non-Hodgkin's lymphoma, or a Hodgkin's lymphoma. In some instances, the hematologic malignancy comprises chronic lymphocytic leukemia (CLL), small lymphocytic lymphoma (SLL), high risk CLL, a non-CLL/SLL lymphoma, prolymphocytic leukemia (PLL), follicular lymphoma (FL), diffuse large B-cell lymphoma (DLBCL), mantle cell lymphoma (MCL), Waldenstrom's macroglobulinemia, multiple myeloma, extranodal marginal zone B cell lymphoma, nodal marginal zone B cell lymphoma, Burkitt's lymphoma, non-Burkitt high grade B cell lymphoma, primary mediastinal B-cell lymphoma (PMBL), immunoblastic large cell lymphoma, precursor B-lymphoblastic lymphoma, B cell prolymphocytic leukemia, lymphoplasmacytic lymphoma, splenic marginal zone lymphoma, plasma cell myeloma, plasmacytoma, mediastinal (thymic) large B cell lymphoma, intravascular large B cell lymphoma, primary effusion lymphoma, or lymphomatoid granulomatosis. In some cases, the hematologic malignancy is a relapsed or refractory hematologic malignancy, or a metastatic hematologic malignancy.

In some instances, the cancer is a KRAS-associated, EGFR-associated, AR-associated cancer, HPRT1-associated cancer, or β-catenin associated cancer. In some instances, a composition or a pharmaceutical formulation described herein comprising a binding moiety conjugated to a polynucleic acid molecule and a polymer is used for the treatment of a KRAS-associated, EGFR-associated, AR-associated cancer, HPRT1-associated cancer, or β-catenin associated cancer. In some instances, a composition or a pharmaceutical formulation described herein comprising a binding moiety conjugated to a polynucleic acid molecule and a polymer is used for the treatment of a KRAS-associated cancer. In some instances, a composition or a pharmaceutical formulation described herein comprising a binding moiety conjugated to a polynucleic acid molecule and a polymer is used for the treatment of an EGFR-associated cancer. In some instances, a composition or a pharmaceutical formulation described herein comprising a binding moiety conjugated to a polynucleic acid molecule and a polymer is used for the treatment of an AR-associated cancer. In some instances, a composition or a pharmaceutical formulation described herein comprising a binding moiety conjugated to a polynucleic acid molecule and a polymer is used for the treatment of an HPRT1-associated cancer. In some instances, a composition or a pharmaceutical formulation described herein comprising a binding moiety conjugated to a polynucleic acid molecule and a polymer is used for the treatment of a β-catenin associated cancer. In some instances, the cancer is a solid tumor. In some instances, the cancer is a hematologic malignancy. In some instances, the solid tumor is a relapsed or refractory solid tumor, or a metastatic solid tumor. In some cases, the hematologic malignancy is a relapsed or refractory hematologic malignancy, or a metastatic hematologic malignancy. In some instances, the cancer comprises bladder cancer, breast cancer, colorectal cancer, endometrial cancer, esophageal cancer, glioblastoma multiforme, head and neck cancer, kidney cancer, lung cancer, ovarian cancer, pancreatic cancer, prostate cancer, thyroid cancer, acute myeloid leukemia, CLL, DLBCL, or multiple myeloma. In some instances, the β-catenin associated cancer further comprises PIK3C-associated cancer and/or MYC-associated cancer.

### Immunotherapy

In some embodiments, a composition or a pharmaceutical formulation described herein is used as an immunotherapy for the treatment of a disease or disorder. In some instances, the immunotherapy is an immuno-oncology therapy. In some instances, immuno-oncology therapy is categorized into active, passive, or combinatory (active and passive) methods. In active immuno-oncology therapy method, for example, tumor-associated antigens (TAAs) are presented to the immune system to trigger an attack on cancer cells presenting these TAAs. In some instances, the active immune-oncology therapy method includes tumor-targeting and/or immune-targeting agents (e.g., checkpoint inhibitor agents such as monoclonal antibodies), and/or vaccines, such as in situ vaccination and/or cell-based or non-cell based (e.g., dendritic cell-based, tumor cell-based, antigen, anti-idiotype, DNA, or vector-based) vaccines. In some instances, the cell-based vaccines are vaccines which are generated using activated immune cells obtained from a patient's own immune system which are then activated by the patient's own cancer. In some instances, the active immune-oncology therapy is further subdivided into non-specific active immunotherapy and specific active immunotherapy. In some instances, non-specific active immunotherapy utilizes cytokines and/or other cell signaling components to induce a general immune system response. In some cases, specific active immunotherapy utilizes specific TAAs to elicite an immune response.

In some embodiments, a composition or a pharmaceutical formulation described herein is used as an active immuno-oncology therapy method for the treatment of a disease or disorder (e.g., cancer). In some embodiments, the composition or a pharmaceutical formulation described herein comprises a tumor-targeting agent. In some instances, the tumor-targeting agent is encompassed by a binding moiety A. In other instances, the tumor-targeting agent is an additional agent used in combination with a molecule of Formula (I). In some instances, the tumor-targeting agent is a tumor-directed polypeptide (e.g., a tumor-directed antibody). In some instances, the tumor-targeting agent is a tumor-directed antibody, which exerts its antitumor activity through mechanisms such as direct killing (e.g., signaling-induced apoptosis), complement-dependent cytotoxicity (CDC), and/or antibody-dependent cell-mediated cytotoxicity (ADCC). In additional instances, the tumor-targeting agent elicits an adaptive immune response, with the induction of antitumor T cells.

In some embodiments, the binding moiety A is a tumor-directed polypeptide (e.g., a tumor-directed antibody). In some instances, the binding moiety A is a tumor-directed antibody, which exerts its antitumor activity through mechanisms such as direct killing (e.g., signaling-induced apoptosis), complement-dependent cytotoxicity (CDC), and/or antibody-dependent cell-mediated cytotoxicity (ADCC). In additional instances, the binding moiety A elicits an adaptive immune response, with the induction of antitumor T cells.

In some embodiments, the composition or a pharmaceutical formulation described herein comprises an immune-targeting agent. In some instances, the immune-targeting agent is encompassed by a binding moiety A. In other instances, the immune-targeting agent is an additional agent used in combination with a molecule of Formula (I). In some instances, the immune-targeting agent comprises cytokines, checkpoint inhibitors, or a combination thereof.

In some embodiments, the immune-targeting agent is a checkpoint inhibitor. In some cases, an immune checkpoint molecule is a molecule presented on the cell surface of CD4 and/or CD8 T cells. Exemplary immune checkpoint molecules include, but are not limited to, Programmed Death-Ligand 1 (PD-L1, also known as B7-H1, CD274), Programmed Death 1 (PD-1), CTLA-4, B7H1, B7H4, OX-40, CD137, CD40, 2B4, IDO1, IDO2, VISTA, CD27, CD28, PD-L2 (B7-DC, CD273), LAG3, CD80, CD86, PDL2, B7H3, HVEM, BTLA, KIR, GAL9, TIM3, A2aR, MARCO (macrophage receptor with collageneous structure), PS (phosphatidylserine), ICOS (inducible T cell costimulator), HAVCR2, CD276, VTCN1, CD70, and CD160.

In some instances, an immune checkpoint inhibitor refers to any molecule that modulates or inhibits the activity of an immune checkpoint molecule. In some instances, immune checkpoint inhibitors include antibodies, antibody-derivatives (e.g., Fab fragments, scFvs, minobodies, diabodies), antisense oligonucleotides, siRNA, aptamers, or peptides. In some embodiments, an immune checkpoint inhibitor is an inhibitor of Programmed Death-Ligand 1 (PD-L1, also known as B7-H1, CD274), Programmed Death 1 (PD-1), CTLA-4, PD-L2 (B7-DC, CD273), LAG3, TIM3, 2B4, A2aR, B7H1, B7H3, B7H4, BTLA, CD2, CD27, CD28, CD30, CD40, CD70, CD80, CD86, CD137, CD160, CD226, CD276, DR3, GAL9, GITR, HAVCR2, HVEM, IDO1, ID02, ICOS (inducible T cell costimulator), KIR, LAIRI, LIGHT, MARCO (macrophage receptor with collageneous structure), PS (phosphatidylserine), OX-40, SLAM, TIGHT, VISTA, VTCN1, or any combinations thereof.

In some embodiments, exemplary checkpoint inhibitors include:

PD-L1 inhibitors such as Genentech's MPDL3280A (RG7446), Anti-mouse PD-L1 antibody Clone 10F.9G2 (Cat #BE0101) from BioXcell, anti-PD-L1 monoclonal antibody MDX-1105 (BMS-936559) and BMS-935559 from Bristol-Meyer's Squibb, MSB0010718C, mouse anti-PD-L1 Clone 29E.2A3, and AstraZeneca's MED14736;

PD-L2 inhibitors such as GlaxoSmithKline's AMP-224 (Amplimmune), and rHIgM12B7;

PD-1 inhibitors such as anti-mouse PD-1 antibody Clone J43 (Cat #BE0033-2) from BioXcell, anti-mouse PD-1 antibody Clone RMP1-14 (Cat #BE0146) from BioXcell, mouse anti-PD-1 antibody Clone EH12, Merck's MK-3475 anti-mouse PD-1 antibody (Keytruda, pembrolizumab, lambrolizumab), AnaptysBio's anti-PD-1 antibody known as ANBO11, antibody MDX-1 106 (ONO-4538), Bristol-Myers Squibb's human IgG4 monoclonal antibody nivolumab (Opdivo®, BMS-936558, MDX1106), AstraZeneca's AMP-514 and AMP-224, and Pidilizumab (CT-O11) from CureTech Ltd;

CTLA-4 inhibitors such as Bristol Meyers Squibb's anti-CTLA-4 antibody ipilimumab (also known as Yervoy®, MDX-010, BMS-734016 and MDX-101), anti-CTLA4 Antibody, clone 9H10 from Millipore, Pfizer's tremelimumab (CP-675,206, ticilimumab), and anti-CTLA4 antibody clone BNI3 from Abcam;

LAG3 inhibitors such as anti-Lag-3 antibody clone eBioC9B7W (C9B7W) from eBioscience, anti-Lag3 antibody LS-B2237 from LifeSpan Biosciences, IMP321 (ImmuFact) from Immutep, anti-Lag3 antibody BMS-986016, and the LAG-3 chimeric antibody A9H12;

B7-H3 inhibitors such as MGA271;

KIR inhibitors such as Lirilumab (IPH2101);

CD137 (41BB) inhibitors such as urelumab (BMS-663513, Bristol-Myers Squibb), PF-05082566 (anti-4-1BB, PF-2566, Pfizer), or XmAb-5592 (Xencor);

PS inhibitors such as Bavituximab;

and inhibitors such as an antibody or fragments (e.g., a monoclonal antibody, a human, humanized, or chimeric antibody) thereof, RNAi molecules, or small molecules to TIM3, CD52, CD30, CD20, CD33, CD27, OX40 (CD134), GITR, ICOS, BTLA (CD272), CD160, 2B4, LAIR1, TIGHT, LIGHT, DR3, CD226, CD2, or SLAM.

In some embodiments, a binding moiety A comprising an immune checkpoint inhibitor is used for the treatment of a disease or disorder (e.g., cancer). In some instances, the binding moiety A is a bispecific antibody or a binding fragment thereof that comprises an immune checkpoint inhibitor. In some cases, a binding moiety A comprising an inhibitor of Programmed Death-Ligand 1 (PD-L1, also known as B7-H1, CD274), Programmed Death 1 (PD-1), CTLA-4, PD-L2 (B7-DC, CD273), LAG3, TIM3, 2B4, A2aR, B7H1, B7H3, B7H4, BTLA, CD2, CD27, CD28, CD30, CD40, CD70, CD80, CD86, CD137, CD160, CD226, CD276, DR3, GAL9, GITR, HAVCR2, HVEM, IDO1, ID02, ICOS (inducible T cell costimulator), KIR, LAIR1, LIGHT, MARCO (macrophage receptor with collageneous structure), PS (phosphatidylserine), OX-40, SLAM, TIGHT, VISTA, VTCN1, or any combinations thereof, is used for the treatment of a disease or disorder (e.g., cancer).

In some embodiments, a molecule of Formula (I) in combination with an immune checkpoint inhibitor is used for the treatment of a disease or disorder (e.g., cancer). In some instances, the immune checkpoint inhibitor comprises an inhibitor of Programmed Death-Ligand 1 (PD-L1, also known as B7-H1, CD274), Programmed Death 1 (PD-1), CTLA-4, PD-L2 (B7-DC, CD273), LAG3, TIM3, 2B4, A2aR, B7H1, B7H3, B7H4, BTLA, CD2, CD27, CD28, CD30, CD40, CD70, CD80, CD86, CD137, CD160, CD226, CD276, DR3, GAL9, GITR, HAVCR2, HVEM, IDO1, ID02, ICOS (inducible T cell costimulator), KIR, LAIR1, LIGHT, MARCO (macrophage receptor with collageneous structure), PS (phosphatidylserine), OX-40, SLAM, TIGHT, VISTA, VTCN1, or any combinations thereof. In some cases, a molecule of Formula (I) is used in combination with ipilimumab, tremelimumab, nivolumab, pemrolizumab, pidilizumab, MPDL3280A, MED14736, MSB0010718C, MK-3475, or BMS-936559, for the treatment of a disease or disorder (e.g., cancer).

In some embodiments, the immune-targeting agent is a cytokine. In some cases, cytokine is further subgrouped into chemokine, interferon, interleukin, and tumor necrosis factor. In some embodiments, chemokine plays a role as a chemoattractant to guide the migration of cells, and is classified into four subfamilies: CXC, CC, CX3C, and XC. Exemplary chemokines include chemokines from the CC subfamily: CCL1, CCL2 (MCP-1), CCL3, CCL4, CCL5 (RANTES), CCL6, CCL7, CCL8, CCL9 (or CCL10), CCL11, CCL12, CCL13, CCL14, CCL15, CCL16, CCL17, CCL18, CCL19, CCL20, CCL21, CCL22, CCL23, CCL24, CCL25, CCL26, CCL27, and CCL28; the CXC subfamily: CXCL1, CXCL2, CXCL3, CXCL4, CXCL5, CXCL6, CXCL7, CXCL8, CXCL9, CXCL10, CXCLi1, CXCL12, CXCL13, CXCL14, CXCL15, CXCL16, and CXCL17; the XC subfamily: XCL1 and XCL2; and the CX3C subfamily CX3CL1.

Interferon (IFNs) comprises interferon type I (e.g. IFN-α, IFN-β, IFN—ε, IFN-κ, and IFN-ω), interferon type II (e.g. IFN-γ), and interferon type III. In some embodiments, IFN-α is further classified into about 13 subtypes which include IFNA, IFNA2, IFNA4, IFNA5, IFNA6, IFNA7, IFNA8, IFNA10, IFNA13, IFNA14, IFNA16, IFNA17, and IFNA21.

Interleukin is expressed by leukocyte or white blood cell and promote the development and differentiation of T and B lymphocytes and hematopoietic cells. Exemplary interleukins include IL-1, IL-2, IL-3, IL-4, IL-51, IL-6, IL-7, IL-8 (CXCL8), IL-9, IL10 IL-11, IL-12, IL-13, IL-14 IL-15, IL-16, IL-17, IL-18, TL-19, IL-20, IL-21, IL-22, IL-23, IL-24, IL-25, IL-26, IL-27, IL-28, IL-29, IL-30, IL-31, IL-32, IL-33, IL-35, and IL-36.

Tumor necrosis factors (TNFs) are a group of cytokines that modulate apoptosis. In some instances, there are about 19 members within the TNF family, including, not limited to, TNFα, lymphotoxin-alpha (LT-alpha), lymphotoxin-beta (LT-beta), T cell antigen gp39 (CD40L), CD27L, CD30L, FASL, 4-IBBL, OX40L, and TNF-related apoptosis inducing ligand (TRAIL).

In some embodiments, a molecule of Formula (I) in combination with a cytokine is used for the treatment of a disease or disorder (e.g., cancer). In some cases, a molecule of Formula (I) in combination with a chemokine is used for the treatment of a disease or disorder (e.g., cancer). In some cases, a molecule of Formula (I) in combination with an interferon is used for the treatment of a disease or disorder (e.g., cancer). In some cases, a molecule of Formula (I) in combination with an interleukin is used for the treatment of a disease or disorder (e.g., cancer). In some cases, a molecule of Formula (I) in combination with a tumor necrosis factor is used for the treatment of a disease or disorder (e.g., cancer). In some instances, a molecule of Formula (I) in combination with IL-1β, IL-2, IL-7, IL-8, IL-15, MCP-1 (CCL2), MIP-1α, RANTES, MCP-3, MIP5, CCL19, CCL21, CXCL2, CXCL9, CXCL10, or CXCL1 1 is used for the treatment of a disease or disorder (e.g., cancer).

In some embodiments, the composition or a pharmaceutical formulation described herein comprises a vaccine. In some instances, the vaccine is an in situ vaccination. In some instances, the vaccine is a cell-based vaccine. In some instances, the vaccine is a non-cell based vaccine. In some instances, a molecule of Formula (I) in combination with dendritic cell-based vaccine is used for the treatment of a disease or disorder (e.g., cancer). In some instances, a molecule of Formula (I) in combination with tumor cell-based vaccine is used for the treatment of a disease or disorder (e.g., cancer). In some instances, a molecule of Formula (I) in combination with antigen vaccine is used for the treatment of a disease or disorder (e.g., cancer). In some instances, a molecule of Formula (I) in combination with anti-idiotype vaccine is used for the treatment of a disease or disorder (e.g., cancer). In some instances, a molecule of Formula (I) in combination with DNA vaccine is used for the treatment of a disease or disorder (e.g., cancer). In some instances, a molecule of Formula (I) in combination with vector-based vaccine is used for the treatment of a disease or disorder (e.g., cancer).

In some embodiments, a composition or a pharmaceutical formulation described herein is used as a passive immuno-oncology therapy method for the treatment of a disease or disorder (e.g., cancer). The passive method, in some instances, utilizes adoptive immune system components such as T cells, natural killer (NK) T cells, and/or chimeric antigen receptor (CAR) T cells generated exogenously to attack cancer cells.

In some embodiments, a molecule of Formula (I) in combination with a T-cell based therapeutic agent is used for the treatment of a disease or disorder (e.g., cancer). In some cases, the T-cell based therapeutic agent is an activated T-cell agent that recognizes one or more of a CD cell surface marker described above. In some instances, the T-cell based therapeutic agent comprises an activated T-cell agent that recognizes one or more of CD2, CD3, CD4, CD5, CD8, CD27, CD28, CD80, CD134, CD137, CD152, CD154, CD160, CD200R, CD223, CD226, CD244, CD258, CD267, CD272, CD274, CD278, CD279, or CD357. In some instances, a molecule of Formula (I) in combination with an activated T-cell agent recognizing one or more of CD2, CD3, CD4, CD5, CD8, CD27, CD28, CD80, CD134, CD137, CD152, CD154, CD160, CD200R, CD223, CD226, CD244, CD258, CD267, CD272, CD274, CD278, CD279, or CD357 is used for the treatment of a disease or disorder (e.g., cancer).

In some embodiments, a molecule of Formula (I) in combination with natural killer (NK) T cell-based therapeutic agent is used for the treatment of a disease or disorder (e.g., cancer). In some instances, the NK-based therapeutic agent is an activated NK agent that recognizes one or more of a CD cell surface marker described above. In some cases, the NK-based therapeutic agent is an activated NK agent that recognizes one or more ofCD2, CD11a, CD11b, CD16, CD56, CD58, CD62L, CD85j, CD158a/b, CD158c, CD158e/f/k, CD158h/j, CD159a, CD162, CD226, CD314, CD335, CD337, CD244, or CD319. In some instances, a molecule of Formula (I) in combination with an activated NK agent recognizing one or more of CD2, CD11a, CD11b, CD16, CD56, CD58, CD62L, CD85j, CD158a/b, CD158c, CD158e/f/k, CD158h/j, CD159a, CD162, CD226, CD314, CD335, CD337, CD244, or CD319 is used for the treatment of a disease or disorder (e.g., cancer).

In some embodiments, a molecule of Formula (I) in combination with CAR-T cell-based therapeutic agent is used for the treatment of a disease or disorder (e.g., cancer).

In some embodiments, a molecule of Formula (I) in combination with an additional agent that destabilizes the endosomal membrane (or disrupts the endosomal-lysosomal membrane trafficking) is used for the treatment of a disease or disorder (e.g., cancer). In some instances, the additional agent comprises an antimitotic agent. Exemplary antimitotic agents include, but are not limited to, taxanes such as paclitaxel and docetaxel; vinca alkaloids such as vinblastine, vincristine, vindesine, and vinorelbine; cabazitaxel; colchicine; eribulin; estramustine; etoposide; ixabepilone; podophyllotoxin; teniposide; or griseofulvin. In some instances, the additional agent comprises paclitaxel, docetaxel, vinblastine, vincristine, vindesine, vinorelbine, cabazitaxel, colchicine, eribulin, estramustine, etoposide, ixabepilone, podophyllotoxin, teniposide, or griseofulvin. In some instances, the additional agent comprises taxol. In some instances, the additional agent comprises paclitaxel. In some instances, the additional agent comprises etoposide. In other instances, the additional agent comprises vitamin K3.

In some embodiments, a composition or a pharmaceutical formulation described herein is used as a combinatory method (including for both active and passive methods) in the treatment of a disease or disorder (e.g., cancer).

### Pharmaceutical Formulation

In some embodiments, the pharmaceutical formulations described herein are administered to a subject by multiple administration routes, including but not limited to, parenteral (e.g., intravenous, subcutaneous, intramuscular), oral, intranasal, buccal, rectal, or transdermal administration routes. In some instances, the pharmaceutical composition describe herein is formulated for parenteral (e.g., intravenous, subcutaneous, intramuscular) administration. In other instances, the pharmaceutical composition describe herein is formulated for oral administration. In still other instances, the pharmaceutical composition describe herein is formulated for intranasal administration.

In some embodiments, the pharmaceutical formulations include, but are not limited to, aqueous liquid dispersions, self-emulsifying dispersions, solid solutions, liposomal dispersions, aerosols, solid dosage forms, powders, immediate-release formulations, controlled-release formulations, fast melt formulations, tablets, capsules, pills, delayed release formulations, extended release formulations, pulsatile release formulations, multiparticulate formulations (e.g., nanoparticle formulations), and mixed immediate and controlled release formulations.

In some instances, the pharmaceutical formulation includes multiparticulate formulations. In some instances, the pharmaceutical formulation includes nanoparticle formulations. In some instances, nanoparticles comprise cMAP, cyclodextrin, or lipids. In some cases, nanoparticles comprise solid lipid nanoparticles, polymeric nanoparticles, self-emulsifying nanoparticles, liposomes, microemulsions, or micellar solutions. Additional exemplary nanoparticles include, but are not limited to, paramagnetic nanoparticles, superparamagnetic nanoparticles, metal nanoparticles, fullerene-like materials, inorganic nanotubes, dendrimers (such as with covalently attached metal chelates), nanofibers, nanohorns, nano-onions, nanorods, nanoropes and quantum dots. In some instances, a nanoparticle is a metal nanoparticle, e.g., a nanoparticle of scandium, titanium, vanadium, chromium, manganese, iron, cobalt, nickel, copper, zinc, yttrium, zirconium, niobium, molybdenum, ruthenium, rhodium, palladium, silver, cadmium, hafnium, tantalum, tungsten, rhenium, osmium, iridium, platinum, gold, gadolinium, aluminum, gallium, indium, tin, thallium, lead, bismuth, magnesium, calcium, strontium, barium, lithium, sodium, potassium, boron, silicon, phosphorus, germanium, arsenic, antimony, and combinations, alloys or oxides thereof.

In some instances, a nanoparticle includes a core or a core and a shell, as in a core-shell nanoparticle.

In some instances, a nanoparticle is further coated with molecules for attachment of functional elements (e.g., with one or more of a polynucleic acid molecule or binding moiety described herein). In some instances, a coating comprises chondroitin sulfate, dextran sulfate, carboxymethyl dextran, alginic acid, pectin, carragheenan, fucoidan, agaropectin, porphyran, karaya gum, gellan gum, xanthan gum, hyaluronic acids, glucosamine, galactosamine, chitin (or chitosan), polyglutamic acid, polyaspartic acid, lysozyme, cytochrome C, ribonuclease, trypsinogen, chymotrypsinogen, α-chymotrypsin, polylysine, polyarginine, histone, protamine, ovalbumin, dextrin, or cyclodextrin. In some instances, a nanoparticle comprises a graphene-coated nanoparticle.

In some cases, a nanoparticle has at least one dimension of less than about 500 nm, 400 nm, 300 nm, 200 nm, or 100 nm.

In some instances, the nanoparticle formulation comprises paramagnetic nanoparticles, superparamagnetic nanoparticles, metal nanoparticles, fullerene-like materials, inorganic nanotubes, dendrimers (such as with covalently attached metal chelates), nanofibers, nanohorns, nano-onions, nanorods, nanoropes or quantum dots. In some instances, a polynucleic acid molecule or a binding moiety described herein is conjugated either directly or indirectly to the nanoparticle. In some instances, at least 1, 5, 10, 15, 20, 30, 40, 50, 60, 70, 80, 90, 100, or more polynucleic acid molecules or binding moieties described herein are conjugated either directly or indirectly to a nanoparticle.

In some embodiments, the pharmaceutical formulations include a carrier or carrier materials selected on the basis of compatibility with the composition disclosed herein, and the release profile properties of the desired dosage form. Exemplary carrier materials include, e.g., binders, suspending agents, disintegration agents, filling agents, surfactants, solubilizers, stabilizers, lubricants, wetting agents, diluents, and the like. Pharmaceutically compatible carrier materials include, but are not limited to, acacia, gelatin, colloidal silicon dioxide, calcium glycerophosphate, calcium lactate, maltodextrin, glycerine, magnesium silicate, polyvinylpyrrollidone (PVP), cholesterol, cholesterol esters, sodium caseinate, soy lecithin, taurocholic acid, phosphotidylcholine, sodium chloride, tricalcium phosphate, dipotassium phosphate, cellulose and cellulose conjugates, sugars sodium stearoyl lactylate, carrageenan, monoglyceride, diglyceride, pregelatinized starch, and the like. See, e.g., Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pennsylvania 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins 1999).

In some instances, the pharmaceutical formulations further include pH-adjusting agents or buffering agents which include acids such as acetic, boric, citric, lactic, phosphoric and hydrochloric acids; bases such as sodium hydroxide, sodium phosphate, sodium borate, sodium citrate, sodium acetate, sodium lactate and tris-hydroxymethylaminomethane; and buffers such as citrate/dextrose, sodium bicarbonate and ammonium chloride. Such acids, bases and buffers are included in an amount required to maintain pH of the composition in an acceptable range.

In some instances, the pharmaceutical formulation includes one or more salts in an amount required to bring osmolality of the composition into an acceptable range. Such salts include those having sodium, potassium or ammonium cations and chloride, citrate, ascorbate, borate, phosphate, bicarbonate, sulfate, thiosulfate or bisulfite anions; suitable salts include sodium chloride, potassium chloride, sodium thiosulfate, sodium bisulfite and ammonium sulfate.

In some instances, the pharmaceutical formulations further include diluent which are used to stabilize compounds because they can provide a more stable environment. Salts dissolved in buffered solutions (which also can provide pH control or maintenance) are utilized as diluents in the art, including, but not limited to a phosphate buffered saline solution. In certain instances, diluents increase bulk of the composition to facilitate compression or create sufficient bulk for homogenous blend for capsule filling. Such compounds can include e.g., lactose, starch, mannitol, sorbitol, dextrose, microcrystalline cellulose such as Avicel®; dibasic calcium phosphate, dicalcium phosphate dihydrate; tricalcium phosphate, calcium phosphate; anhydrous lactose, spray-dried lactose; pregelatinized starch, compressible sugar, such as Di-Pac® (Amstar); mannitol, hydroxypropylmethylcellulose, hydroxypropylmethylcellulose acetate stearate, sucrose-based diluents, confectioner's sugar; monobasic calcium sulfate monohydrate, calcium sulfate dihydrate; calcium lactate trihydrate, dextrates; hydrolyzed cereal solids, amylose; powdered cellulose, calcium carbonate; glycine, kaolin; mannitol, sodium chloride; inositol, bentonite, and the like.

In some cases, the pharmaceutical formulations include disintegration agents or disintegrants to facilitate the breakup or disintegration of a substance. The term “disintegrate” include both the dissolution and dispersion of the dosage form when contacted with gastrointestinal fluid. Examples of disintegration agents include a starch, e.g., a natural starch such as corn starch or potato starch, a pregelatinized starch such as National 1551 or Amijel®, or sodium starch glycolate such as Promogel® or Explotab®, a cellulose such as a wood product, methylcrystalline cellulose, e.g., Avicel®, Avicel® PH101, Avicel® PH102, Avicel® PH105, Elcema® P100, Emcocel®, Vivacel®, Ming Tia®, and Solka-Floc®, methylcellulose, croscarmellose, or a cross-linked cellulose, such as cross-linked sodium carboxymethylcellulose (Ac-Di-Sol®), cross-linked carboxymethylcellulose, or cross-linked croscarmellose, a cross-linked starch such as sodium starch glycolate, a cross-linked polymer such as crospovidone, a cross-linked polyvinylpyrrolidone, alginate such as alginic acid or a salt of alginic acid such as sodium alginate, a clay such as Veegum® HV (magnesium aluminum silicate), a gum such as agar, guar, locust bean, Karaya, pectin, or tragacanth, sodium starch glycolate, bentonite, a natural sponge, a surfactant, a resin such as a cation-exchange resin, citrus pulp, sodium lauryl sulfate, sodium lauryl sulfate in combination starch, and the like.

In some instances, the pharmaceutical formulations include filling agents such as lactose, calcium carbonate, calcium phosphate, dibasic calcium phosphate, calcium sulfate, microcrystalline cellulose, cellulose powder, dextrose, dextrates, dextran, starches, pregelatinized starch, sucrose, xylitol, lactitol, mannitol, sorbitol, sodium chloride, polyethylene glycol, and the like.

Lubricants and glidants are also optionally included in the pharmaceutical formulations described herein for preventing, reducing or inhibiting adhesion or friction of materials. Exemplary lubricants include, e.g., stearic acid, calcium hydroxide, talc, sodium stearyl fumerate, a hydrocarbon such as mineral oil, or hydrogenated vegetable oil such as hydrogenated soybean oil (Sterotex®), higher fatty acids and their alkali-metal and alkaline earth metal salts, such as aluminum, calcium, magnesium, zinc, stearic acid, sodium stearates, glycerol, talc, waxes, Stearowet®, boric acid, sodium benzoate, sodium acetate, sodium chloride, leucine, a polyethylene glycol (e.g., PEG-4000) or a methoxypolyethylene glycol such as Carbowax™, sodium oleate, sodium benzoate, glyceryl behenate, polyethylene glycol, magnesium or sodium lauryl sulfate, colloidal silica such as Syloid™, Cab-O-Sil®, a starch such as corn starch, silicone oil, a surfactant, and the like.

Plasticizers include compounds used to soften the microencapsulation material or film coatings to make them less brittle. Suitable plasticizers include, e.g., polyethylene glycols such as PEG 300, PEG 400, PEG 600, PEG 1450, PEG 3350, and PEG 800, stearic acid, propylene glycol, oleic acid, triethyl cellulose and triacetin. Plasticizers can also function as dispersing agents or wetting agents.

Solubilizers include compounds such as triacetin, triethylcitrate, ethyl oleate, ethyl caprylate, sodium lauryl sulfate, sodium doccusate, vitamin E TPGS, dimethylacetamide, N-methylpyrrolidone, N-hydroxyethylpyrrolidone, polyvinylpyrrolidone, hydroxypropylmethyl cellulose, hydroxypropyl cyclodextrins, ethanol, n-butanol, isopropyl alcohol, cholesterol, bile salts, polyethylene glycol 200-600, glycofurol, transcutol, propylene glycol, dimethyl isosorbide, and the like.

Stabilizers include compounds such as any antioxidation agents, buffers, acids, preservatives and the like.

Suspending agents include compounds such as polyvinylpyrrolidone, e.g., polyvinylpyrrolidone K12, polyvinylpyrrolidone K17, polyvinylpyrrolidone K25, or polyvinylpyrrolidone K30, vinyl pyrrolidone/vinyl acetate copolymer (S630), polyethylene glycol, e.g., the polyethylene glycol can have a molecular weight of about 300 to about 6000, or about 3350 to about 4000, or about 7000 to about 5400, sodium carboxymethylcellulose, methylcellulose, hydroxypropylmethylcellulose, hydroxymethylcellulose acetate stearate, polysorbate-80, hydroxyethylcellulose, sodium alginate, gums, such as, e.g., gum tragacanth and gum acacia, guar gum, xanthans, including xanthan gum, sugars, cellulosics, such as, e.g., sodium carboxymethylcellulose, methylcellulose, sodium carboxymethylcellulose, hydroxypropylmethylcellulose, hydroxyethylcellulose, polysorbate-80, sodium alginate, polyethoxylated sorbitan monolaurate, polyethoxylated sorbitan monolaurate, povidone and the like.

Surfactants include compounds such as sodium lauryl sulfate, sodium docusate, Tween 60 or 80, triacetin, vitamin E TPGS, sorbitan monooleate, polyoxyethylene sorbitan monooleate, polysorbates, polaxomers, bile salts, glyceryl monostearate, copolymers of ethylene oxide and propylene oxide, e.g., Pluronic® (BASF), and the like. Additional surfactants include polyoxyethylene fatty acid glycerides and vegetable oils, e.g., polyoxyethylene (60) hydrogenated castor oil; and polyoxyethylene alkylethers and alkylphenyl ethers, e.g., octoxynol 10, octoxynol 40. Sometimes, surfactants is included to enhance physical stability or for other purposes.

Viscosity enhancing agents include, e.g., methyl cellulose, xanthan gum, carboxymethyl cellulose, hydroxypropyl cellulose, hydroxypropylmethyl cellulose, hydroxypropylmethyl cellulose acetate stearate, hydroxypropylmethyl cellulose phthalate, carbomer, polyvinyl alcohol, alginates, acacia, chitosans and combinations thereof.

Wetting agents include compounds such as oleic acid, glyceryl monostearate, sorbitan monooleate, sorbitan monolaurate, triethanolamine oleate, polyoxyethylene sorbitan monooleate, polyoxyethylene sorbitan monolaurate, sodium docusate, sodium oleate, sodium lauryl sulfate, sodium doccusate, triacetin, Tween 80, vitamin E TPGS, ammonium salts and the like.

### Therapeutic Regimens

In some embodiments, the pharmaceutical compositions described herein are administered for therapeutic applications. In some embodiments, the pharmaceutical composition is administered once per day, twice per day, three times per day or more. The pharmaceutical composition is administered daily, every day, every alternate day, five days a week, once a week, every other week, two weeks per month, three weeks per month, once a month, twice a month, three times per month, or more. The pharmaceutical composition is administered for at least 1 month, 2 months, 3 months, 4 months, 5 months, 6 months, 7 months, 8 months, 9 months, 10 months, 11 months, 12 months, 18 months, 2 years, 3 years, or more.

In some embodiments, one or more pharmaceutical compositions are administered simutaneously, sequentially, or at an interval period of time. In some embodiments, one or more pharmaceutical compositions are administered simutaneously. In some cases, one or more pharmaceutical compositions are administered sequentially. In additional cases, one or more pharmaceutical compositions are administered at an interval period of time (e.g., the first administration of a first pharmaceutical composition is on day one followed by an interval of at least 1, 2, 3, 4, 5, or more days prior to the administration of at least a second pharmaceutical composition).

In some embodiments, two or more different pharmaceutical compositions are coadministered. In some instances, the two or more different pharmaceutical compositions are coadministered simutaneously. In some cases, the two or more different pharmaceutical compositions are coadministered sequentially without a gap of time between administrations. In other cases, the two or more different pharmaceutical compositions are coadministered sequentially with a gap of about 0.5 hour, 1 hour, 2 hour, 3 hour, 12 hours, 1 day, 2 days, or more between administrations.

In the case wherein the patient's status does improve, upon the doctor's discretion the administration of the composition is given continuously; alternatively, the dose of the composition being administered is temporarily reduced or temporarily suspended for a certain length of time (i.e., a “drug holiday”). In some instances, the length of the drug holiday varies between 2 days and 1 year, including by way of example only, 2 days, 3 days, 4 days, 5 days, 6 days, 7 days, 10 days, 12 days, 15 days, 20 days, 28 days, 35 days, 50 days, 70 days, 100 days, 120 days, 150 days, 180 days, 200 days, 250 days, 280 days, 300 days, 320 days, 350 days, or 365 days. The dose reduction during a drug holiday is from 10%-100%, including, by way of example only, 10%, 15%, 20%, 25%, 30%, 35%, 40%, 45%, 50%, 55%, 60%, 65%, 70%, 75%, 80%, 85%, 90%, 95%, or 100%.

Once improvement of the patient's conditions has occurred, a maintenance dose is administered if necessary. Subsequently, the dosage or the frequency of administration, or both, are optionally reduced, as a function of the symptoms, to a level at which the improved disease, disorder or condition is retained.

In some embodiments, the amount of a given agent that correspond to such an amount varies depending upon factors such as the particular compound, the severity of the disease, the identity (e.g., weight) of the subject or host in need of treatment, but nevertheless is routinely determined in a manner known in the art according to the particular circumstances surrounding the case, including, e.g., the specific agent being administered, the route of administration, and the subject or host being treated. In some instances, the desired dose is conveniently presented in a single dose or as divided doses administered simultaneously (or over a short period of time) or at appropriate intervals, for example as two, three, four or more sub-doses per day.

The foregoing ranges are merely suggestive, as the number of variables in regard to an individual treatment regime is large, and considerable excursions from these recommended values are not uncommon. Such dosages are altered depending on a number of variables, not limited to the activity of the compound used, the disease or condition to be treated, the mode of administration, the requirements of the individual subject, the severity of the disease or condition being treated, and the judgment of the practitioner.

In some embodiments, toxicity and therapeutic efficacy of such therapeutic regimens are determined by standard pharmaceutical procedures in cell cultures or experimental animals, including, but not limited to, the determination of the LD50 (the dose lethal to 50% of the population) and the ED50 (the dose therapeutically effective in 50% of the population). The dose ratio between the toxic and therapeutic effects is the therapeutic index and it is expressed as the ratio between LD50 and ED50. Compounds exhibiting high therapeutic indices are preferred. The data obtained from cell culture assays and animal studies are used in formulating a range of dosage for use in human. The dosage of such compounds lies preferably within a range of circulating concentrations that include the ED50 with minimal toxicity. The dosage varies within this range depending upon the dosage form employed and the route of administration utilized.

### Kits/Article of Manufacture

Disclosed herein, in certain embodiments, are kits and articles of manufacture for use with one or more of the compositions and methods described herein. Such kits include a carrier, package, or container that is compartmentalized to receive one or more containers such as vials, tubes, and the like, each of the container(s) comprising one of the separate elements to be used in a method described herein. Suitable containers include, for example, bottles, vials, syringes, and test tubes. In one embodiment, the containers are formed from a variety of materials such as glass or plastic.

The articles of manufacture provided herein contain packaging materials. Examples of pharmaceutical packaging materials include, but are not limited to, blister packs, bottles, tubes, bags, containers, bottles, and any packaging material suitable for a selected formulation and intended mode of administration and treatment.

For example, the container(s) include a molecule of Formula (I): A-X—B—Y—C, optionally conjugated to an endosomolytic moiety D as disclosed herein. Such kits optionally include an identifying description or label or instructions relating to its use in the methods described herein.

A kit typically includes labels listing contents and/or instructions for use and package inserts with instructions for use. A set of instructions will also typically be included.

In one embodiment, a label is on or associated with the container. In one embodiment, a label is on a container when letters, numbers, or other characters forming the label are attached, molded or etched into the container itself; a label is associated with a container when it is present within a receptacle or carrier that also holds the container, e.g., as a package insert. In one embodiment, a label is used to indicate that the contents are to be used for a specific therapeutic application. The label also indicates directions for use of the contents, such as in the methods described herein.

In certain embodiments, the pharmaceutical compositions are presented in a pack or dispenser device which contains one or more unit dosage forms containing a compound provided herein. The pack, for example, contains metal or plastic foil, such as a blister pack. In one embodiment, the pack or dispenser device is accompanied by instructions for administration. In one embodiment, the pack or dispenser is also accompanied with a notice associated with the container in form prescribed by a governmental agency regulating the manufacture, use, or sale of pharmaceuticals, which notice is reflective of approval by the agency of the form of the drug for human or veterinary administration. Such notice, for example, is the labeling approved by the U.S. Food and Drug Administration for prescription drugs, or the approved product insert. In one embodiment, compositions containing a compound provided herein formulated in a compatible pharmaceutical carrier are also prepared, placed in an appropriate container, and labeled for treatment of an indicated condition.

### Certain Terminology

Unless defined otherwise, all technical and scientific terms used herein have the same meaning as is commonly understood by one of skill in the art to which the claimed subject matter belongs. It is to be understood that the foregoing general description and the following detailed description are exemplary and explanatory only and are not restrictive of any subject matter claimed. In this application, the use of the singular includes the plural unless specifically stated otherwise. It must be noted that, as used in the specification and the appended claims, the singular forms “a,” “an” and “the” include plural referents unless the context clearly dictates otherwise. In this application, the use of “or” means “and/or” unless stated otherwise. Furthermore, use of the term “including” as well as other forms, such as “include”, “includes,” and “included,” is not limiting.

As used herein, ranges and amounts can be expressed as “about” a particular value or range. About also includes the exact amount. Hence “about 5 μL” means “about 5 μL” and also “5 μL.” Generally, the term “about” includes an amount that is expected to be within experimental error.

The section headings used herein are for organizational purposes only and are not to be construed as limiting the subject matter described.

As used herein, the terms “individual(s)”, “subject(s)” and “patient(s)” mean any mammal. In some embodiments, the mammal is a human. In some embodiments, the mammal is a non-human. None of the terms require or are limited to situations characterized by the supervision (e.g. constant or intermittent) of a health care worker (e.g. a doctor, a registered nurse, a nurse practitioner, a physician's assistant, an orderly or a hospice worker).

### EXAMPLES

These examples are provided for illustrative purposes only and not to limit the scope of the claims provided herein.

### Chemical Synthesis Examples

### Example 1. Preparation of Compound 1-3, and 5-8

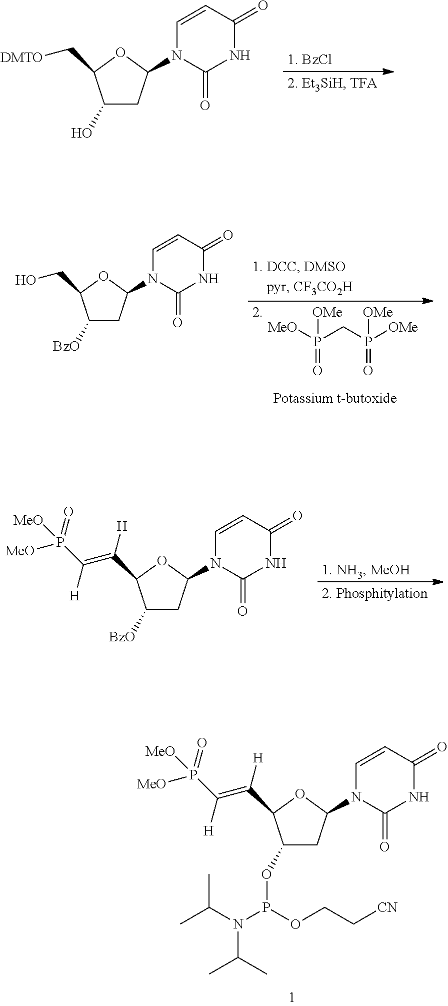

or as follows

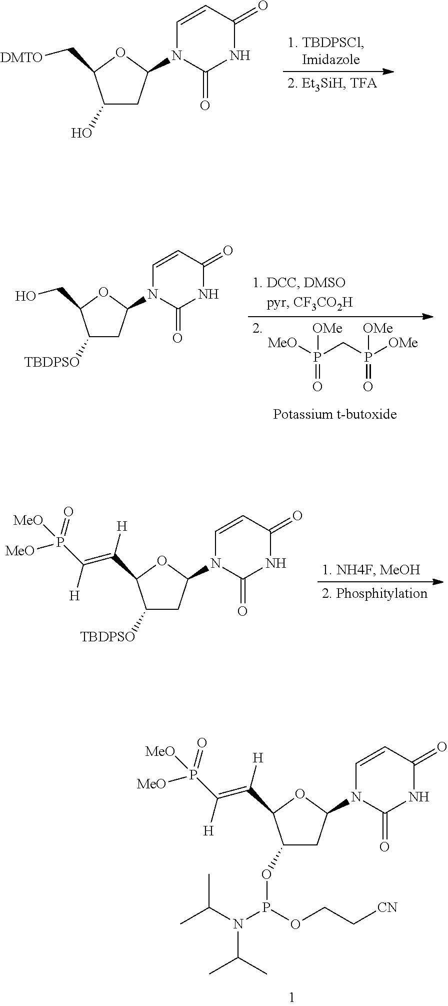

Compounds 2, 3, and 5-8 were prepared as per procedures illustrated in Example 1.

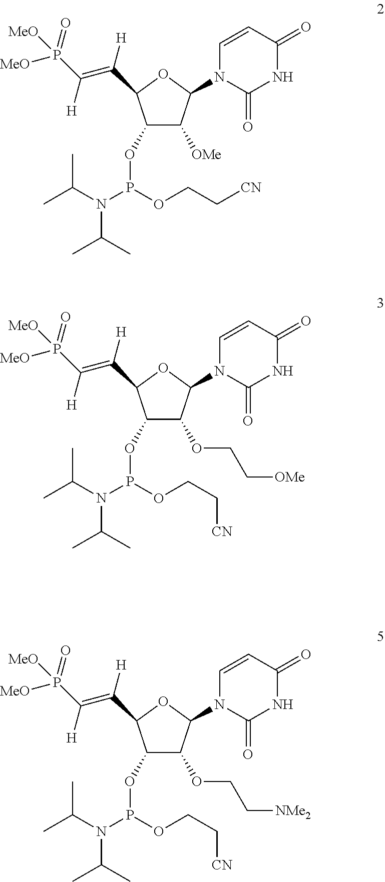 [SMILES: CO[C@@H]1[C@H](OP(OCCC#N)N(C(C)C)C(C)C)[C@@H](/C=C/P(=O)(OC)OC)O[C@H]1n1ccc(=O)[nH]c1=O ; COCCO[C@@H]1[C@H](OP(OCCC#N)N(C(C)C)C(C)C)[C@@H](/C=C/P(=O)(OC)OC)O[C@H]1n1ccc(=O)[nH]c1=O ; COP(=O)(/C=C/[C@H]1O[C@@H](n2ccc(=O)[nH]c2=O)[C@H](OCCN(C)C)[C@@H]1OP(OCCC#N)N(C(C)C)C(C)C)OC]

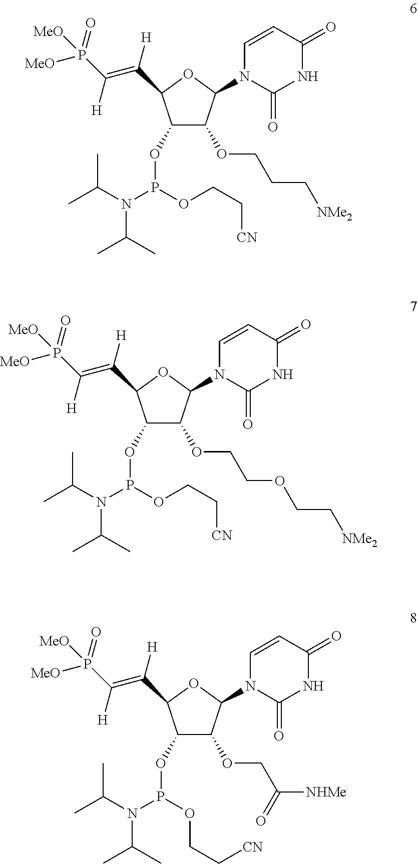 [SMILES: COP(=O)(/C=C/[C@H]1O[C@@H](n2ccc(=O)[nH]c2=O)[C@H](OCCCN(C)C)[C@@H]1OP(OCCC#N)N(C(C)C)C(C)C)OC ; COP(=O)(/C=C/[C@H]1O[C@@H](n2ccc(=O)[nH]c2=O)[C@H](OCCOCCN(C)C)[C@@H]1OP(OCCC#N)N(C(C)C)C(C)C)OC ; CNC(=O)CO[C@@H]1[C@H](OP(OCCC#N)N(C(C)C)C(C)C)[C@@H](/C=C/P(=O)(OC)OC)O[C@H]1n1ccc(=O)[nH]c1=O]

### Example 2. Preparation of Compound 4

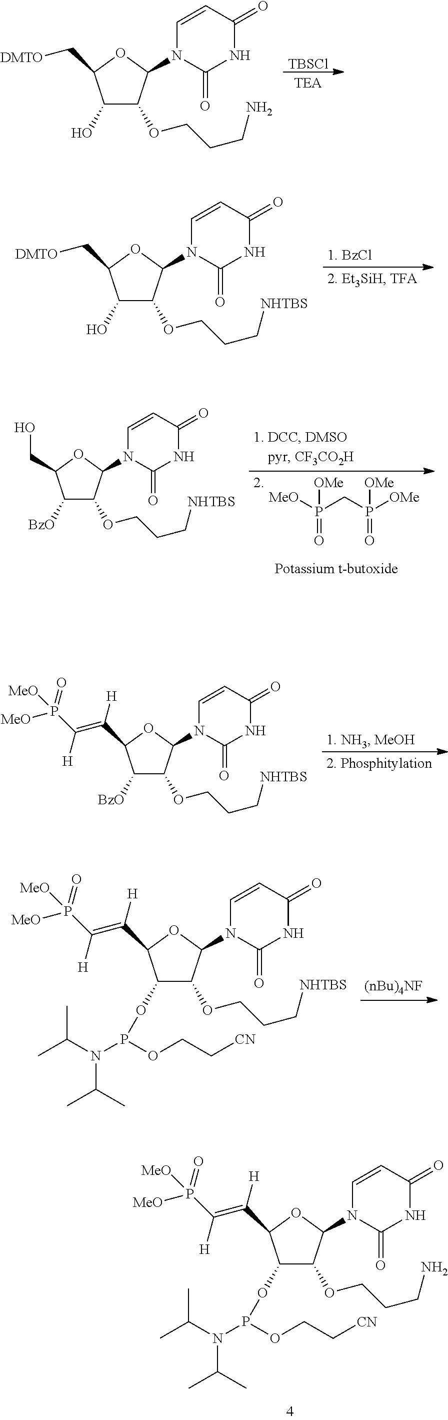

or as follows:

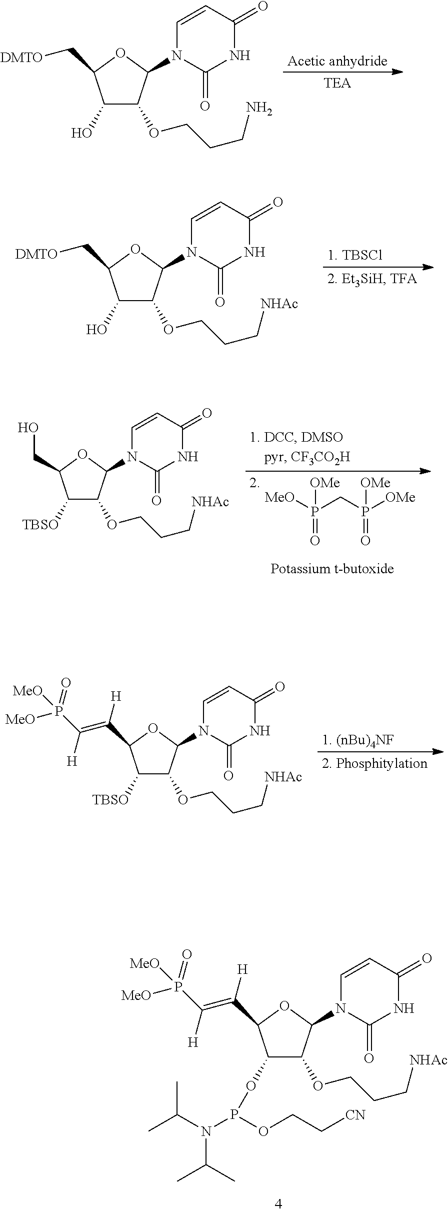

### Example 3. Preparation of Compound 9

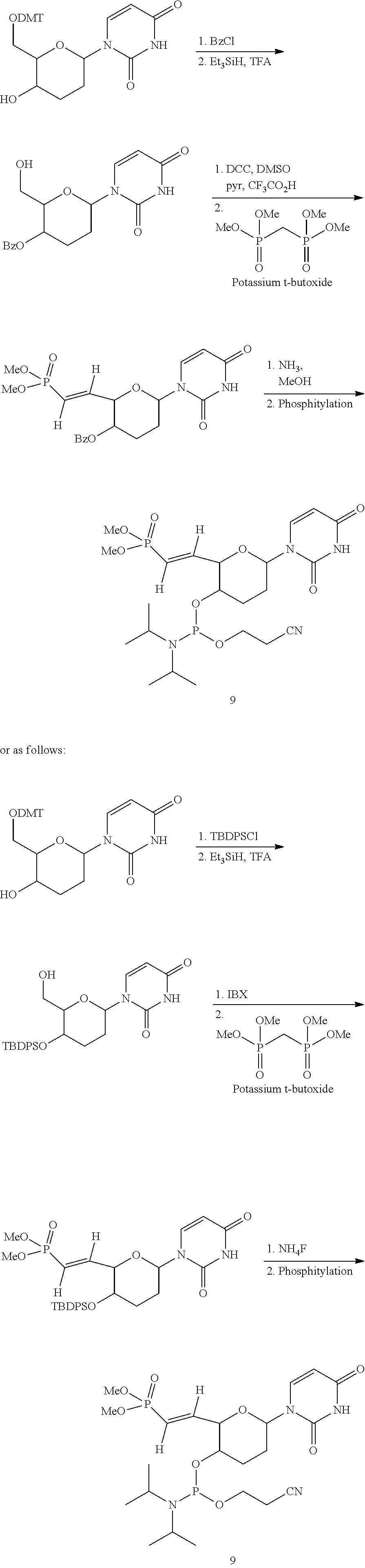

### Example 4. Preparation of Compound 10

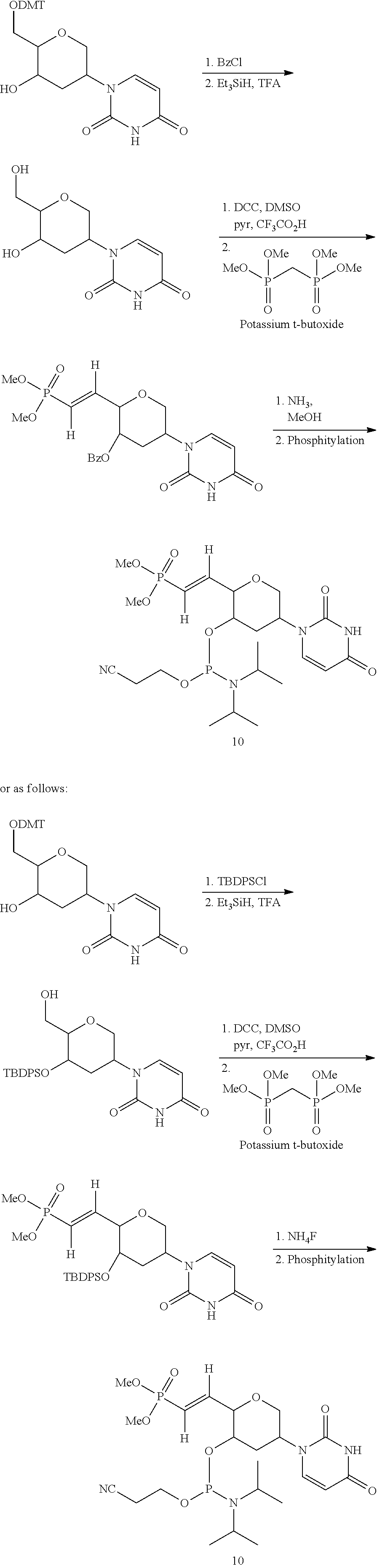

### Example 5. Preparation of Compound 11

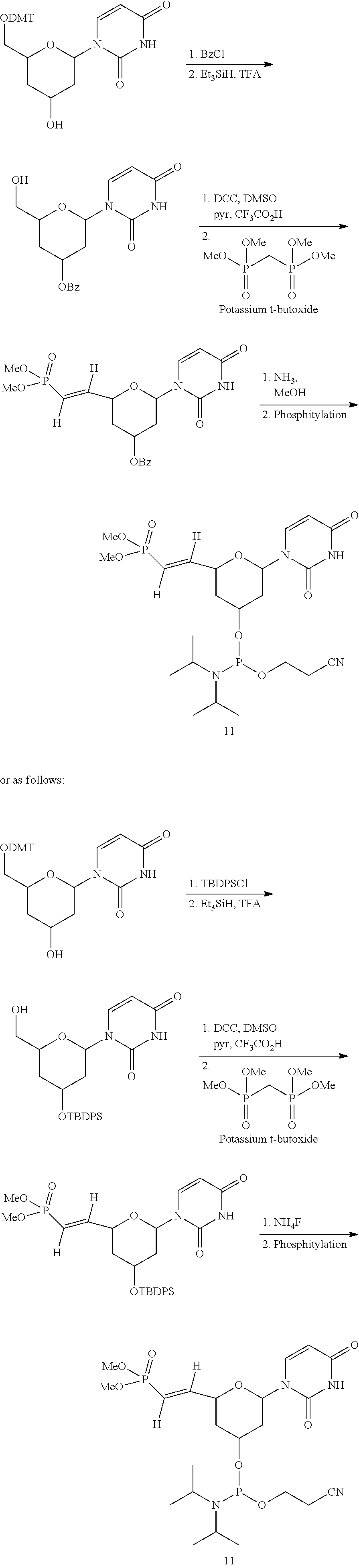

### Example 6. Preparation of Compound 12

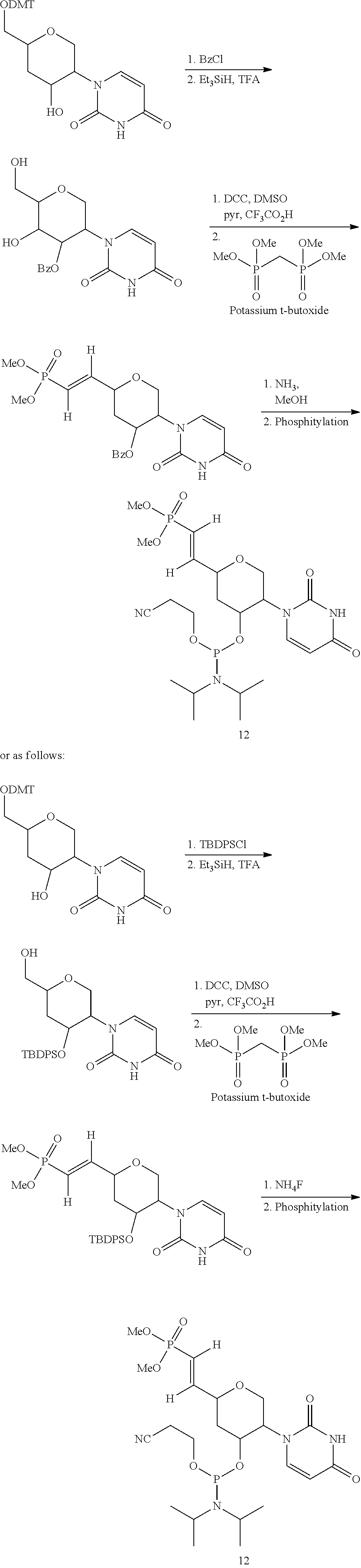

### Example 7. Preparation of Compound 14

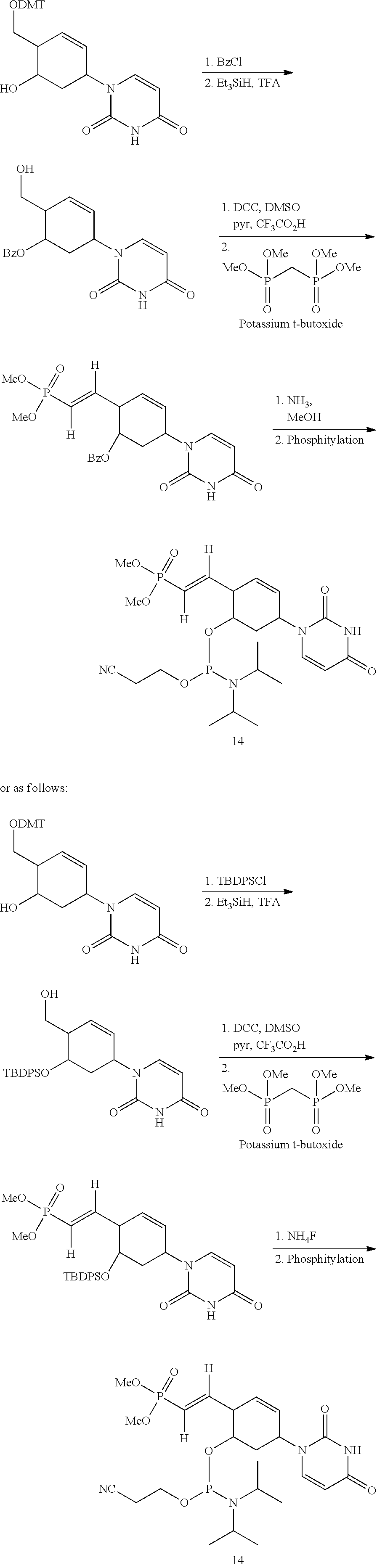

### Example 8. Preparation of Compound 15

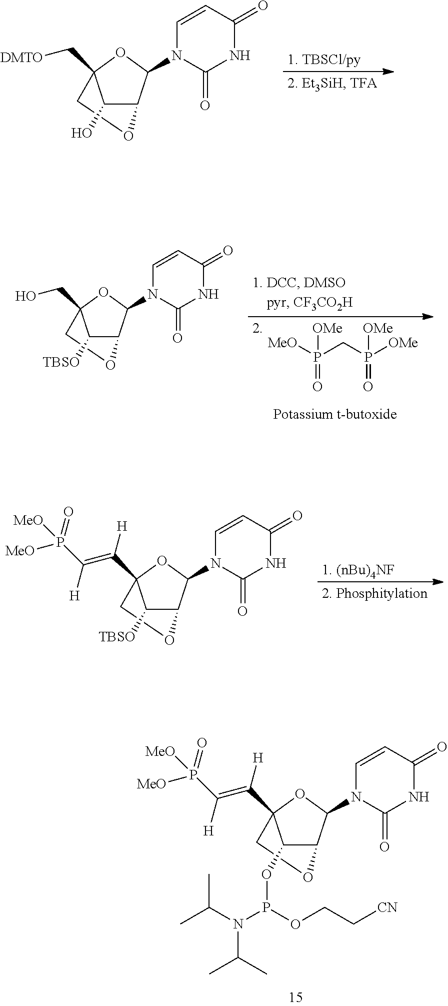

### Example 10. Preparation of Compound 17

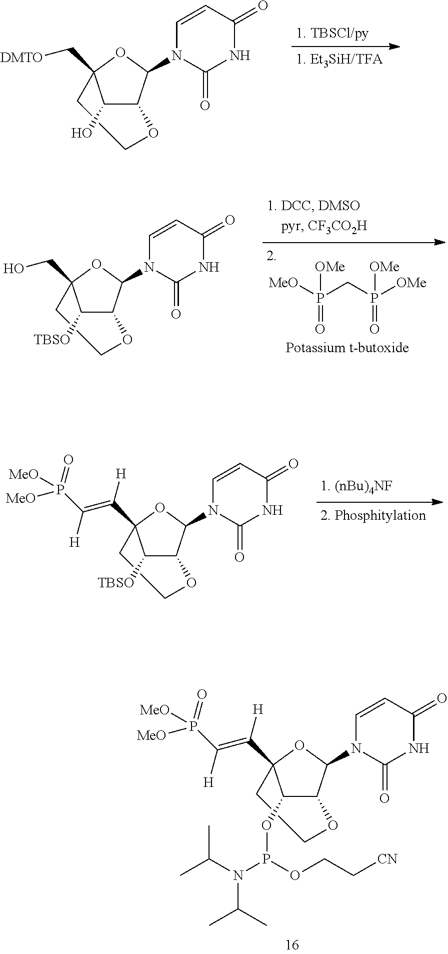

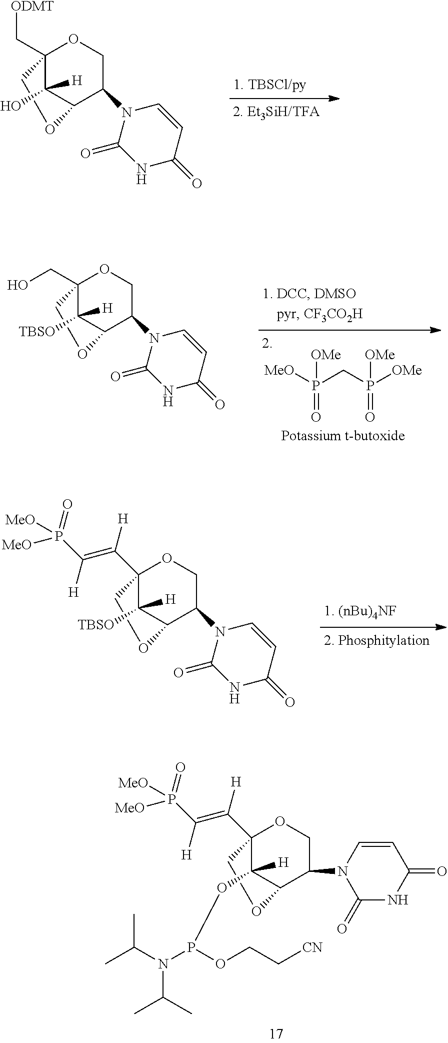

### Example 11. Preparation of Compound 18

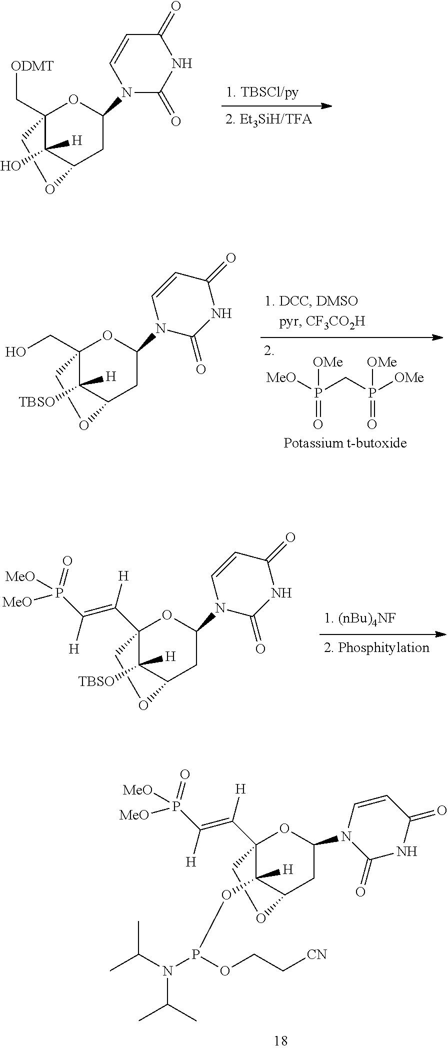

### Example 12. Preparation of Compound 19

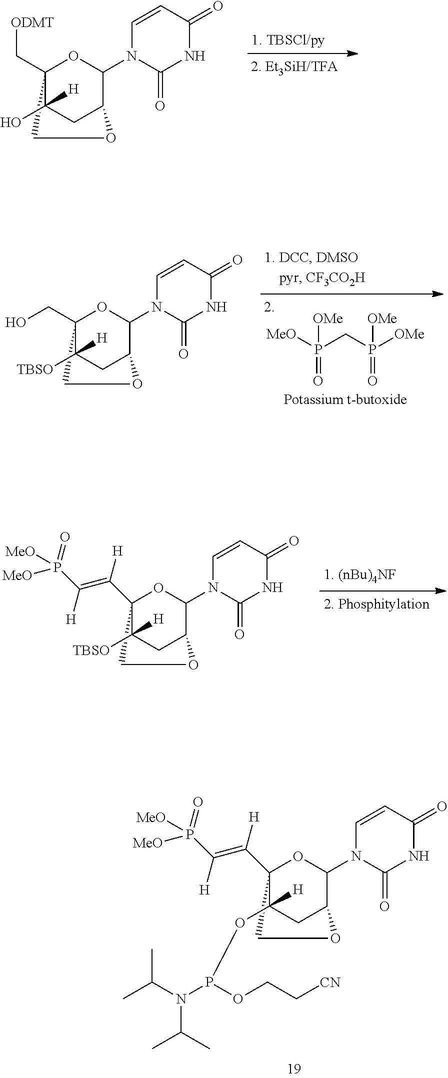

### Example 13. Preparation of Compound 20

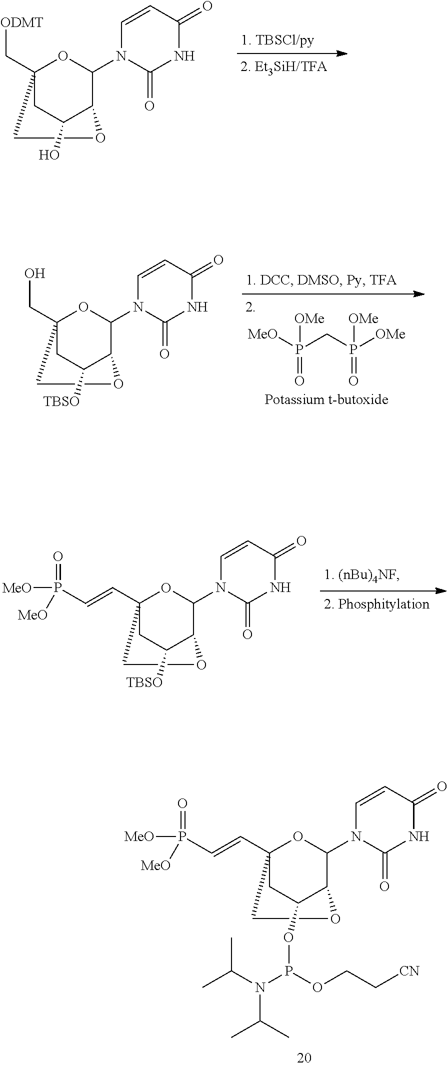

### Example 14. Preparation of Compound 21

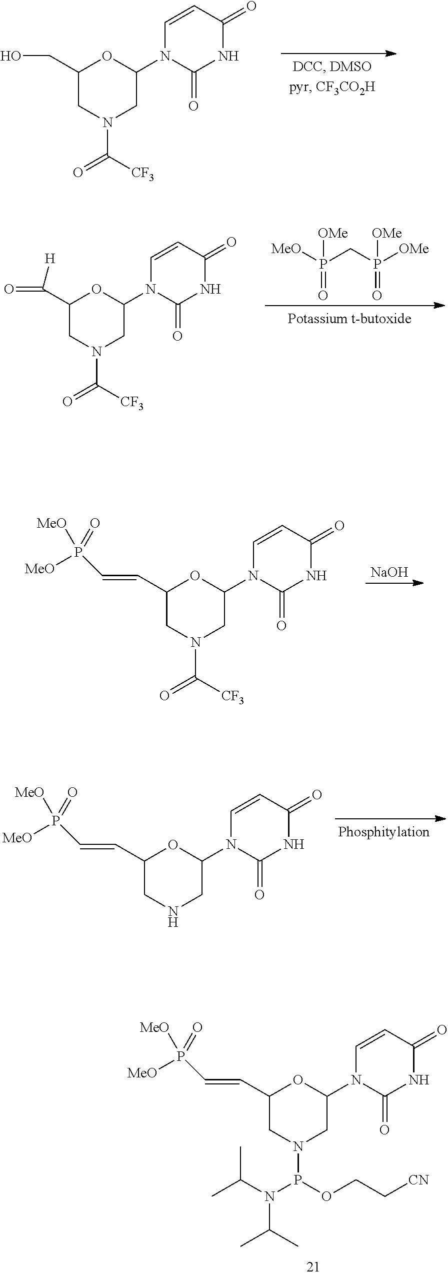

### Example 15. Preparation of Compound 22

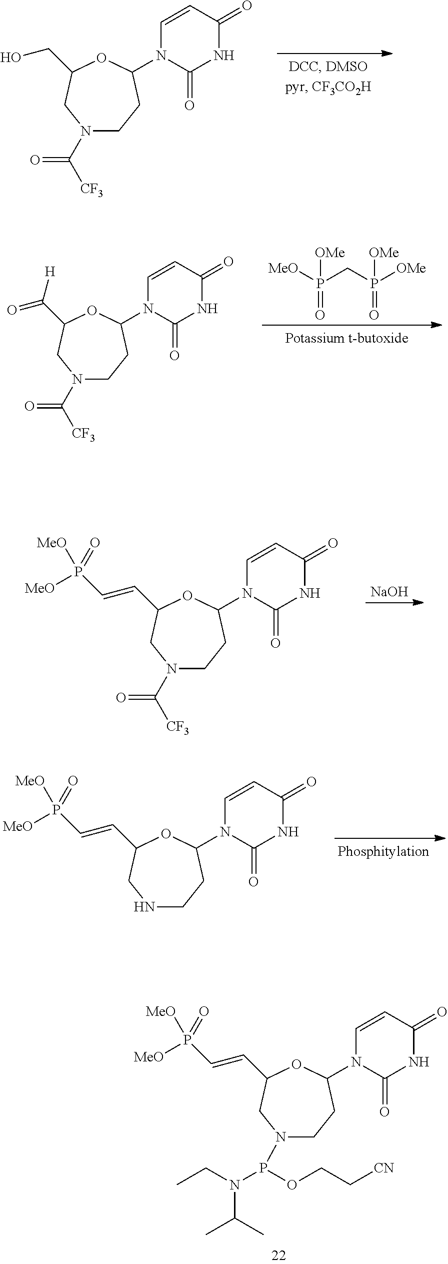

### Example 16. Preparation of Compound 23

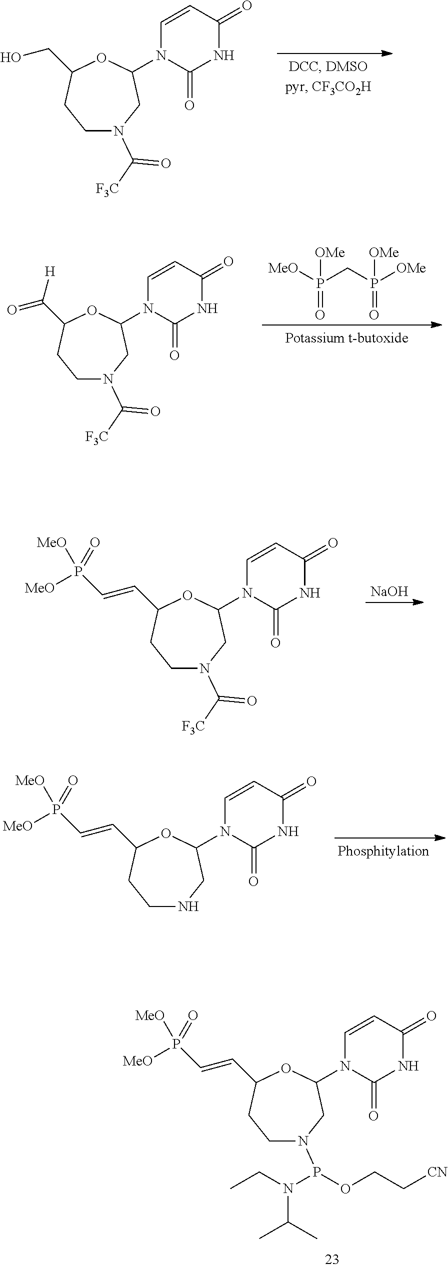

### Example 17. Preparation of Compound 24

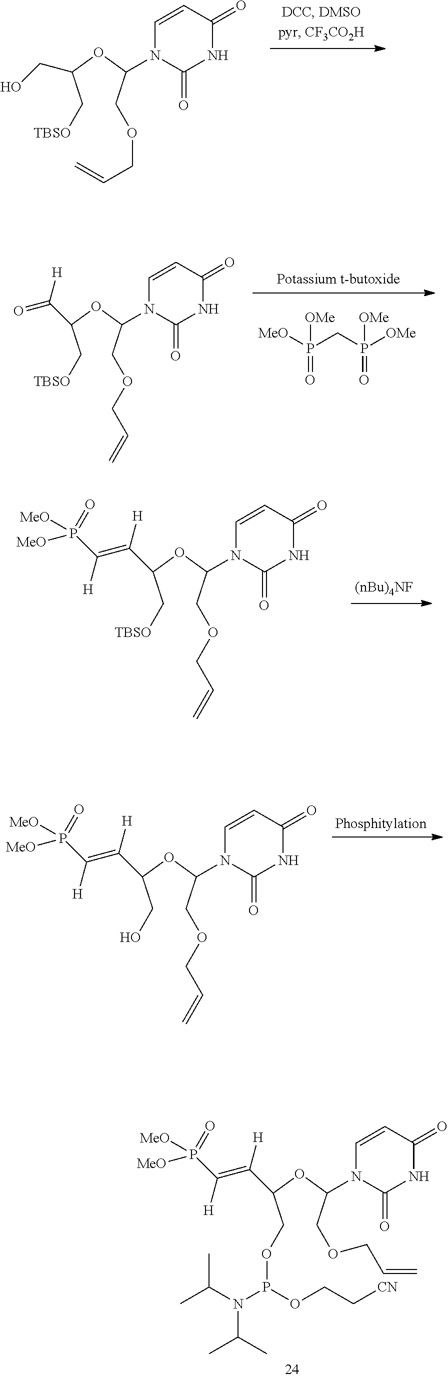 [SMILES: C=CCOCC(OC(CO)CO[Si](C)(C)C(C)(C)C)n1ccc(=O)[nH]c1=O ; C=CCOCC(OC(C=O)CO[Si](C)(C)C(C)(C)C)n1ccc(=O)[nH]c1=O ; COP(=O)(CP(=O)(OC)OC)OC ; C=CCOCC(OC(/C=C/P(=O)(OC)OC)CO[Si](C)(C)C(C)(C)C)n1ccc(=O)[nH]c1=O ; C=CCOCC(OC(/C=C/P(=O)(OC)OC)CO)n1ccc(=O)[nH]c1=O ; C=CCOCC(OC(/C=C/P(=O)(OC)OC)COP(OCCC#N)N(C(C)C)C(C)C)n1ccc(=O)[nH]c1=O]

### Example 18. Preparation of Compound 25

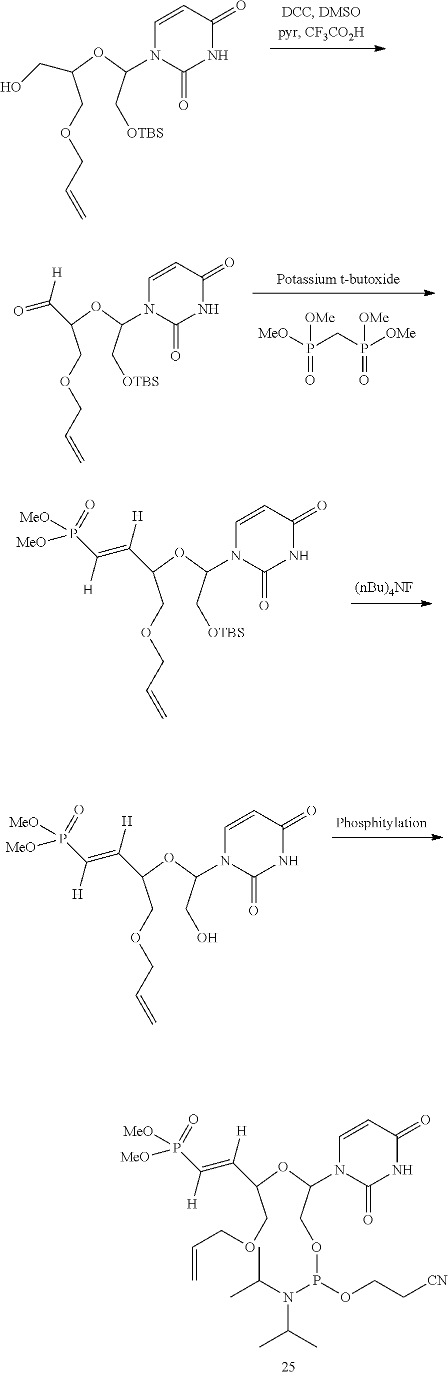

### Example 19. Preparation of Compound 26

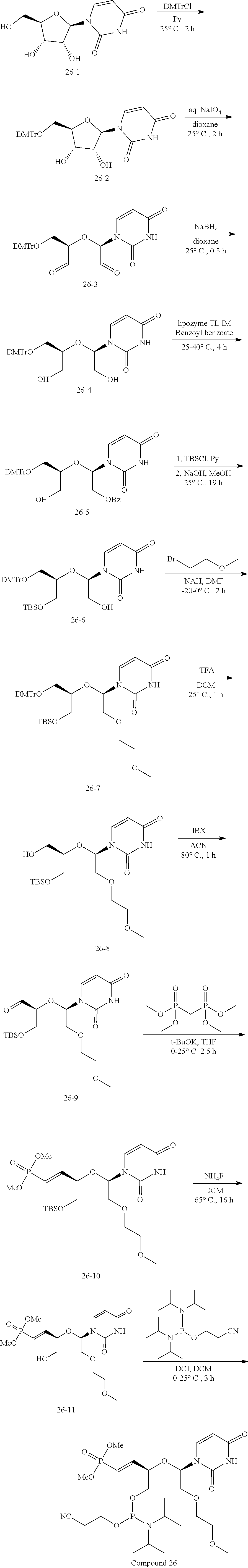

### Synthetic Procedures for the Preparation of Compound 26

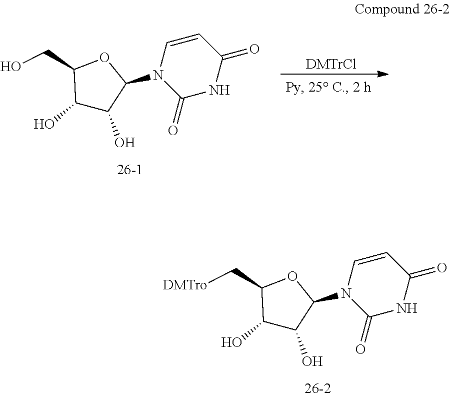

To a solution of compound 26-1 (500 g, 2.05 mol, 1.00 eq) in Py (3500 mL) was added DMTrCl (763 g, 2.25 mol, 1.10 eq). The mixture was stirred at 25° C. for 2 h. TLC (DCM/MeOH=10/1, compound 26-2: R_{f}=0.60) indicated compound 26-1 was consumed completely. The reaction mixture was diluted with DCM (5.00 L) and washed with NaHCO₃ (2.00 L×2). The combined organic layers were washed with Brine (2.00 L), dried over Na₂SO₄, filtered and concentrated under reduced pressure to give a residue. The residue was purified by column chromatography (SiO₂, Petroleum ether/Ethyl acetate/TEA=50/1/0.5% to 0/1/0.5%). Compound 26-2 (700 g, 56.3% yield, 90.0% purity) was obtained as a white solid.

¹H NMR: 400 MHz, DMSO-d₆ δ ppm 11.35 (s, 1H), 7.72 (d, J=8.0 Hz, 1H), 7.36-7.41 (m, 2H), 7.32 (t, J=7.6 Hz, 2H), 7.20-7.28 (m, 5H), 6.90 (d, J=8.6 Hz, 4H), 5.48 (d, J=4.8 Hz, 1H), 5.31 (d, J=8.0 Hz, 1H), 5.14 (d, J=5.6 Hz, 1H), 4.09 (q, J=5.4 Hz, 2H), 3.92-3.99 (m, 1H), 3.70-3.78 (m, 6H), 3.18-3.30 (m, 2H).

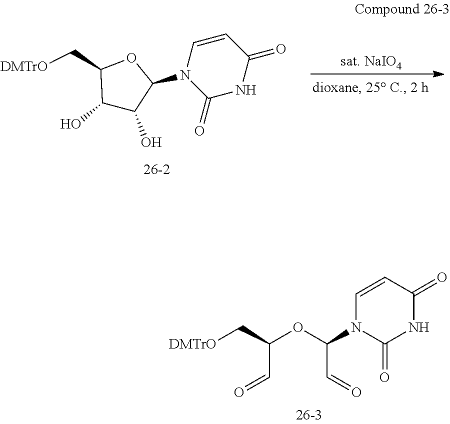

To a solution of compound 26-2 (250 g, 457 mmol, 1.00 eq) in dioxane (2500 mL) was added sat NaIO₄ (103 g, 484 mmol, 4.85 mL, 1.06 eq) solution. The mixture was stirred at 25° C. for 2 h. TLC (DCM/MeOH=10/1, compound 26-3: R_{f}=0.49) indicated compound 26-2 was consumed completely. The reaction mixture was filtered. The crude product compound 26-3 (250 g) was obtained as a colorless oil and used in the next step without further purification.

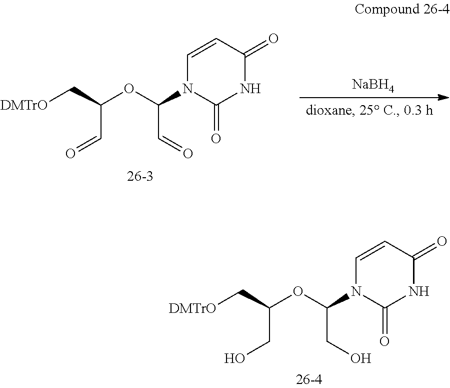

To a solution of compound 26-3 (250 g, 459 mmol, 1.00 eq) in dioxane (3000 mL) was added NaBH₄ (17.3 g, 459 mmol, 1.00 eq). The mixture was stirred at 25° C. for 0.3 h. TLC (DCM/MeOH=10/1, compound 26-4: R_{f}=0.41) indicated compound 26-3 was consumed completely. The reaction mixture was quenched with acetone, neutralized with 20% acetic acid, and concentrated to give a residue under reduced pressure. The residue was diluted with DCM (2.00 L), washed with H₂O (2.00 L), dried over Na₂SO₄, filtered and concentrated under reduced pressure to give a residue. The product was used into the next step without further purification. Compound 26-4 (250 g) was obtained as a colorless oil.

¹H NMR: 400 MHz, DMSO-d₆ δ ppm 11.35 (s, 1H), 7.65 (d, J=8.0 Hz, 1H), 7.28-7.33 (m, 4H), 7.17-7.21 (m, 5H), 6.86 (d, J=8.0 Hz, 4H), 5.83 (t, J=6.4 Hz, 1H), 5.53 (d, J=8.0 Hz, 1H), 5.13 (t, J=8.0 Hz, 1H), 4.75 (t, J=5.2 Hz, 1H), 3.76 (s, 6H), 3.68-3.73 (m, 1H), 3.60-3.64 (m, 2H), 3.42 (t, J=5.2 Hz, 2H), 2.96-3.02 (m, 2H)

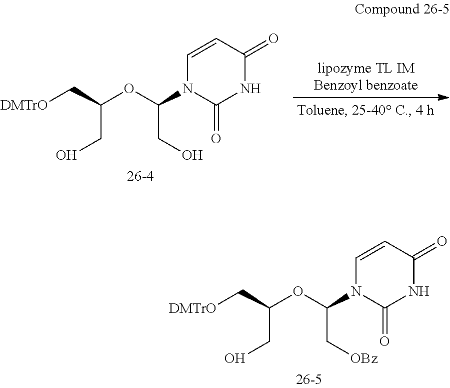

To a solution of compound 26-4 (50.0 g, 91.1 mmol, 1.00 eq) in toluene (2500 mL) was added benzoyl benzoate (30.9 g, 136 mmol, 25.7 mL, 1.50 eq) and lipozyme TL IM (30.0 g, 29.2 mmol) at 25° C. The mixture was stirred at 40° C. for 4 h. TLC (DCM/MeOH=20/1, compound 26-5: R_{f}=0.58) indicated compound 26-4 was consumed completely. The reaction mixture was filtered, quenched by addition methanol (250 mL), concentrated under reduced pressure to give a residue, which was purified by column chromatography (SiO₂, Petroleum ether/Ethyl acetate/TEA=10/1/0.5% to 1/2/0.5%). Compound 26-5 (37.5 g, 63.0% yield) was obtained as a white solid.

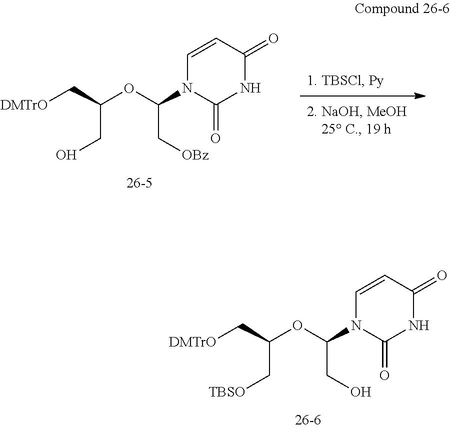

To a solution of compound 26-5 (340 g, 520 mmol, 1.00 eq) in Py (1700 mL) was added TBSCl (157 g, 1.04 mol, 127 mL, 2.00 eq) below 25° C. The mixture was stirred at 25° C. for 19 h. TLC (DCM/MeOH=20/1, compound 26-6: R_{f}=0.37) indicated compound 26-5 was consumed completely. Water (340 mL) was added to the reaction mixture. The resulting mixture containing the nucleoside was dissolved in MeOH (1.00 L), then added dropwise NaOH in MeOH (1.00 L, pH=10) at 0° C., then stirred for 2.5 h at 0° C. The mixture was concentrated under reduced pressure to give a residue. Sat. aq NH₄Cl (1.00 L) was added to the mixture, which was stirred for 10 min. Water (1.00 L) was added and the mixture extracted with DCM (1.00 L), dried over Na₂SO₄, filtered and concentrated under reduced pressure to give a residue, which was purified by column chromatography (SiO₂, Petroleum ether/Ethyl acetate/TEA=10/1/0.5% to 0/1/0.5%). Compound 26-6 (250 g, 339 mmol, 65.2% yield, 90.0% purity) was obtained as a white solid.

¹H NMR: 400 MHz, DMSO-d₆ δ ppm 11.35 (s, 1H), 7.65 (d, J=8.0 Hz, 1H), 7.24-7.33 (m, 4H), 7.14-7.22 (m, 5H), 6.82-6.88 (m, 4H), 5.83 (t, J=5.8 Hz, 1H), 5.47-5.57 (m, 1H), 5.11 (t, J=5.8 Hz, 1H), 3.72 (d, J=0.8 Hz, 6H), 3.57-3.67 (m, 4H), 3.46-3.55 (m, 1H), 2.94-3.00 (m, 2H), 0.72-0.78 (m, 9H), −0.04 (d, J=8.2 Hz, 5H)

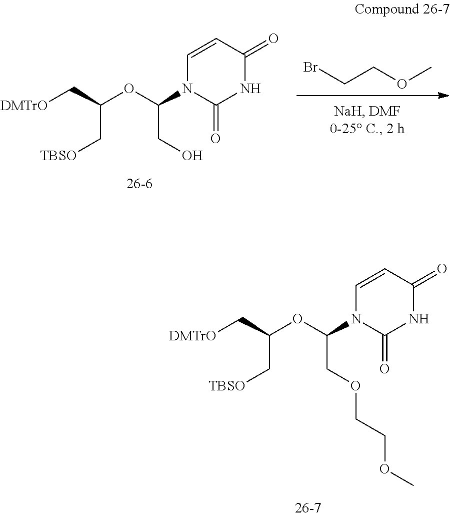

To a solution of compound 26-6 (50.0 g, 75.4 mmol, 1.00 eq) in DMF (500 mL) was added NaH (9.96 g, 248. mmol, 1.81 uL, 60% purity, 3.30 eq) at −20° C. and stirred at −20° C. for 0.5 h. Then the alkoxy bromide (15.7 g, 113. mmol, 10.6 mL, 1.50 eq) was added to the reaction. The mixture was stirred at 0° C. for 1.5 h. TLC (Petroleum ether/Ethyl acetate=1/2, compound 7: R_{f}=0.43) indicated compound 26-6 was consumed completely. The reaction mixture was quenched by addition NH₄Cl (1.00 L×2) at 25° C., and then diluted with ethyl acetate (500 mL) and washed with water (500 mL). The combined organic layers were washed with brine (500 mL), dried over Na₂SO₄, filtered and concentrated under reduced pressure to give a residue. The residue was purified by column chromatography (SiO₂, Petroleum ether/Ethyl acetate/TEA=10/1/0.5% to 0/1/0.5%). Compound 26-7 (34.0 g, 47.1 mmol, 62.5% yield) was obtained as a colorless oil.

¹H NMR: 400 MHz DMSO-d₆ δ ppm 11.40 (d, J=1.8 Hz, 1H), 7.68 (d, J=8.2 Hz, 1H), 7.24-7.33 (m, 5H), 7.14-7.22 (m, 5H), 6.81-6.88 (m, 4H), 5.96-6.02 (m, 1H), 5.55 (dd, J=8.0, 2.0 Hz, 1H), 3.73 (d, J=0.8 Hz, 6H), 3.70 (dd, J=6.0, 2.2 Hz, 2H), 3.62-3.66 (m, 2H), 3.53-3.57 (m, 2H), 3.50-3.53 (m, 1H), 3.38-3.42 (m, 2H), 3.20-3.22 (m, 3H), 2.94-3.00 (m, 2H), 0.72-0.82 (m, 9H), −0.04 (d, J=7.4 Hz, 5H)

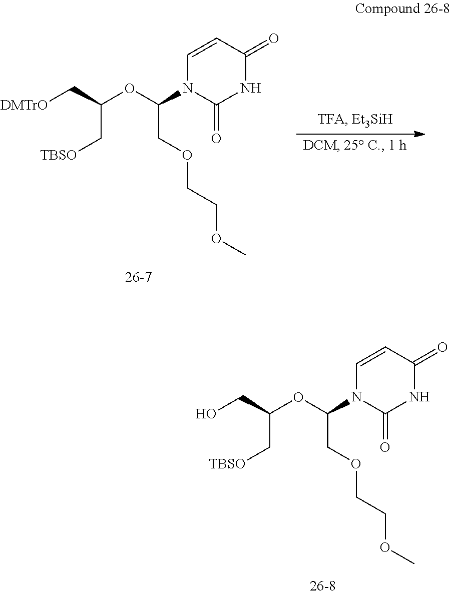

To a solution of compound 26-7 (46.8 g, 64.9 mmol, 1.00 eq) in DCM (468 mL) was added TFA (43.1 g, 378 mmol, 28.0 mL, 5.83 eq) and Et₃SiH (15.1 g, 129 mmol, 20.7 mL, 2.00 eq). The mixture was stirred at 25° C. for 1 hr. TLC (Petroleum ether/Ethyl acetate=0/1, compound 26-8: R_{f}=0.43) indicated compound 26-7 was consumed completely. The reaction mixture was diluted with DCM (500 mL) and washed with H20 (500 mL). The combined organic layers were washed with NaHCO₃ (500 mL×3) and brine (500 mL), dried over Na₂SO₄, filtered and concentrated under reduced pressure to give a residue. The residue was purified by column chromatography (SiO₂, Petroleum ether/Ethyl acetate=10/1 to 0/1). Compound 26-8 (17.0 g, 36.5 mmol, 56.3% yield, 90.0% purity) was obtained as a colorless oil.

¹H NMR: 400 MHz, DMSO-d₆ δ ppm 11.87 (s, 1H), 8.17 (d, J=8.2 Hz, 1H), 7.75-7.87 (m, 4H), 7.63-7.74 (m, 5H), 7.36 (dd, J=9.0, 2.6 Hz, 4H), 6.34 (t, J=5.8 Hz, 1H), 6.05 (d, J=8.0 Hz, 1H), 5.62 (t, J=5.8 Hz, 1H), 4.24 (s, 6H), 4.09-4.20 (m, 4H), 4.02 (d, J=4.6 Hz, 1H), 3.83 (s, 1H), 2.95-3.09 (m, 4H), 1.28 (s, 9H), 0.48 (d, J=8.2 Hz, 6H).

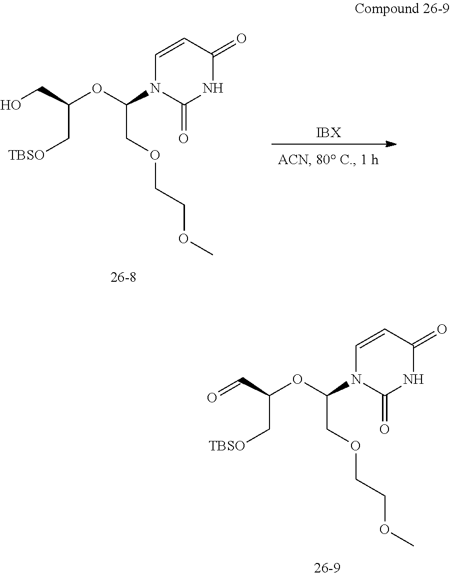

To a solution of compound 26-8 (17.0 g, 40.6 mmol, 1.00 eq) in ACN (170 mL) was added IBX (17.1 g, 60.9 mmol, 1.50 eq). The mixture was stirred at 80° C. for 1 hr. LC-MS showed compound 26-8 was consumed completely. The reaction mixture was filtered and concentrated under reduced pressure to give a residue. The crude product compound 26-9 (17.0 g, crude) was obtained as a colorless oil and used in the next step without further purification.

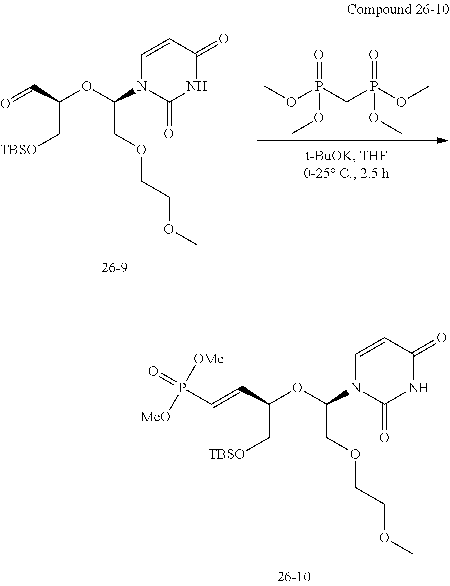

To a solution of compound tetramethyl methylenediphosphonate (15.1 g, 65.3 mmol, 1.60 eq) in THF (100 mL) was added dropwise t-BuOK (6.87 g, 61.2 mmol, 1.50 eq) at 0° C., and the mixture stirred at 25° C. for 0.5 hr. The mixture was then added dropwise to compound 26-9 (17.0 g, 40.8 mmol, 1.00 eq) in THE (70.0 mL) and stirred at 0° C. for 1 h, allowed to reach 25° C. and stirred at 25° C. for 1 h. TLC (Petroleum ether/Ethyl acetate=0/1, compound 10: R_{f}=0.07) indicated compound 26-9 was consumed completely. The reaction mixture was quenched by addition NH₄Cl (500 mL), and then diluted with ethyl acetate (200 mL) and extracted. The combined organic layers were washed with brine (200 mL), dried over Na₂SO₄, filtered and concentrated under reduced pressure to give a residue. The residue was purified by column chromatography (SiO₂, petroleum ether/ethyl acetate=10/1 to 0/1). Compound 26-10 (11.4 g, 16.3 mmol, 40.1% yield, 75.0% purity) was obtained as a white solid.

¹H NMR: 400 MHz, CDCl₃ δ ppm 9.19 (s, 1H), 7.43 (d, J=8.16 Hz, 1H), 6.51-6.67 (m, 1H), 6.13 (t, J=4.78 Hz, 1H), 5.82-5.95 (m, 1H), 5.72 (dd, J=8.04, 1.88 Hz, 1H), 4.20 (s, 1H), 3.63-3.78 (m, 12H), 3.45-3.54 (m, 2H), 3.31-3.37 (m, 3H), 0.82-0.94 (m, 9H), 0.00-0.10 (m, 6H).

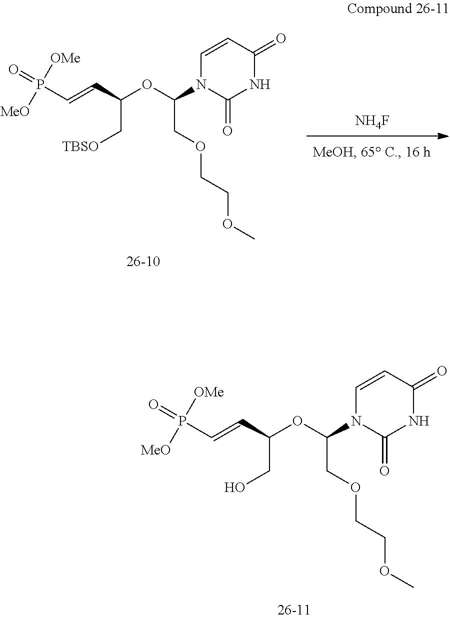 [SMILES: COCCOC[C@@H](O[C@H](/C=C/P(=O)(OC)OC)CO[Si](C)(C)C(C)(C)C)n1ccc(=O)[nH]c1=O ; "NH4F" ; COCCOC[C@@H](O[C@H](/C=C/P(=O)(OC)OC)CO)n1ccc(=O)[nH]c1=O]

To a solution of compound 26-10 (11.4 g, 21. mmol, 1.00 eq) in methanol (114 mL) was added NH₄F (6.46 g, 174 mmol, 8.00 eq). The mixture was stirred at 65° C. for 16 h. TLC (DCM/MeOH=20/1, compound 10: R_{f}=0.28) indicated compound 26-10 was consumed completely. The reaction mixture was concentrated under reduced pressure to give a residue. The residue was purified by column chromatography (SiO₂, Ethyl acetate/MeOH=100/1 to 10/1). Compound 26-11 (5.90 g, 13.0 mmol, 59.6% yield, 90.0% purity) was obtained as a colorless oil.

¹H NMR: 400 MHz DMSO-d₆, δ ppm 11.29 (s, 1H) 7.74 (d, J=8.0 Hz, 1H) 6.42-6.62 (m, 1H) 5.90-5.98 (m, 2H) 5.62 (dd, J=8.0, 1.2 Hz, 1H) 5.01 (t, J=5.6 Hz, 1H) 4.18 (d, J=1.6 Hz, 1H) 4.10 (q, J=5.2 Hz, 1H) 3.70-3.76 (m, 2H) 3.50-3.58 (m, 8H) 3.43-3.49 (m, 1H) 3.36-3.41 (m, 2H) 3.21 (s, 3H) 3.17 (d, J=5.2 Hz, 2H) 1.91 (s, 1H)

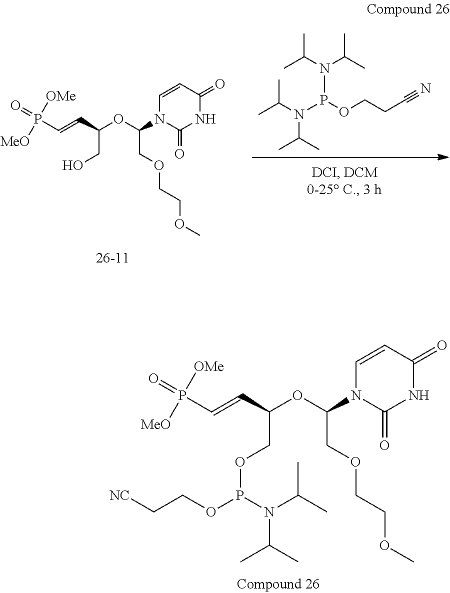 [SMILES: COCCOC[C@@H](O[C@H](/C=C/P(=O)(OC)OC)CO)n1ccc(=O)[nH]c1=O ; CC(C)N(C(C)C)P(OCCC#N)N(C(C)C)C(C)C ; COCCOC[C@@H](O[C@H](/C=C/P(=O)(OC)OC)COP(OCCC#N)N(C(C)C)C(C)C)n1ccc(=O)[nH]c1=O]

To a solution of compound 26-11 (4.90 g, 12.0 mmol, 1.00 eq) in DCM (49.0 mL) was added DCI (2.27 g, 19.2 mmol, 1.60 eq) and 2-Cyanoethyl N,N,N′,N-tetraisopropylphosphorodiamidite (6.51 g, 21.6 mmol, 6.86 mL, 1.80 eq) at 0° C. The mixture was stirred at 25° C. for 3 hrs. TLC (DCM/MeOH=10/1, Compound 26: R_{f}=0.58) indicated compound 26-11 was consumed completely. The reaction mixture was diluted with DCM (50.0 mL) and washed with aq. NaHCO₃ (50.0 mL×2). The combined organic layers were washed with brine (50.0 mL), dried over Na₂SO₄, filtered and concentrated under reduced pressure to give a residue. The residue was purified by column chromatography (SiO₂, Petroleum ether/Ethyl acetate/TEA=100/1/0.5% to 0/1/0.5%). Compound 26 (5.40 g, 8.52 mmol, 70.9% yield, 96.0% purity) was obtained as a colorless oil.

³¹P decoupled ¹H NMR: (400 MHz, CD₃CN): δ 7.42-7.45 (dd, J=8 Hz, 1H), δ 6.42-6.47 (dd, J=17.2 Hz, 1H), 5.97-5.84 (m, 2H), 5.54-5.52 (d, J=8 Hz, 1H), 4.27-4.23 (m, 1H), 3.75-3.50 (br, 12H), 3.36-3.30 (br, 2H), 3.18 (s, 3H), 2.60-2.57 (m, 2H), 1.10-1.09 (d, 12H)

³¹P NMR (400 MHz, CD₃CN) δ148.6, 148.4, 19.24, 19.22

MS (ESI) calculated for C₂₄H₄₂N₄O₁₀P₂ (M-H)⁻ m/z=607.2, found 607.2

### Example 20. Preparation of Compound 27

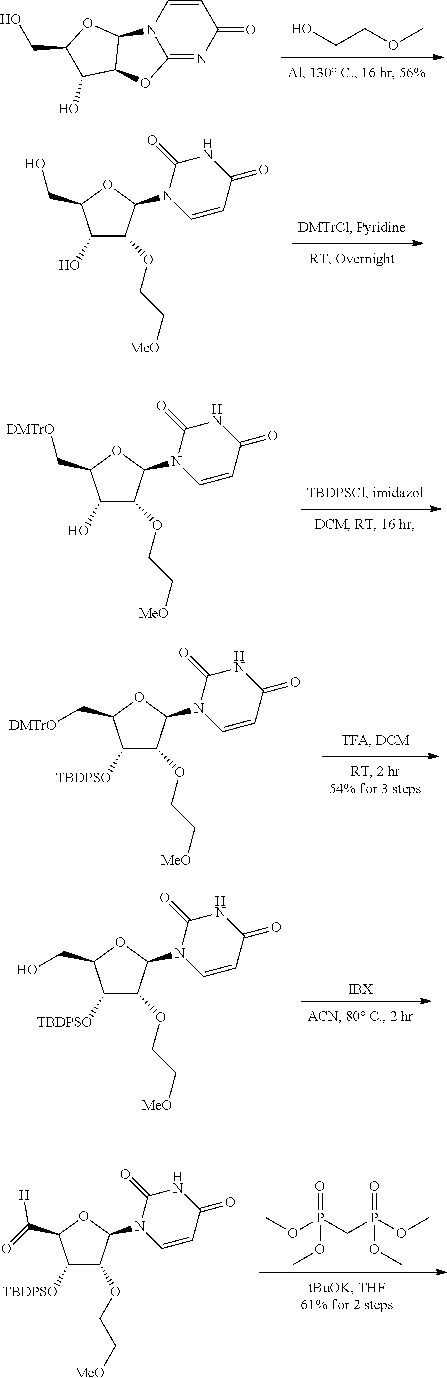

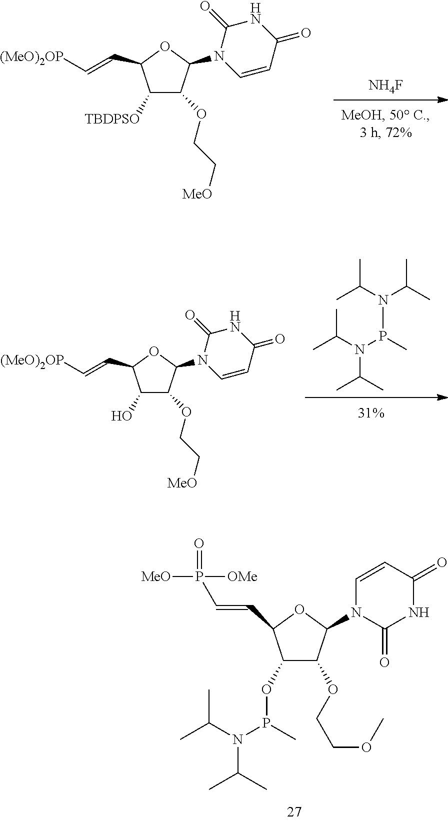

### Example 21. Preparation of Compound 28

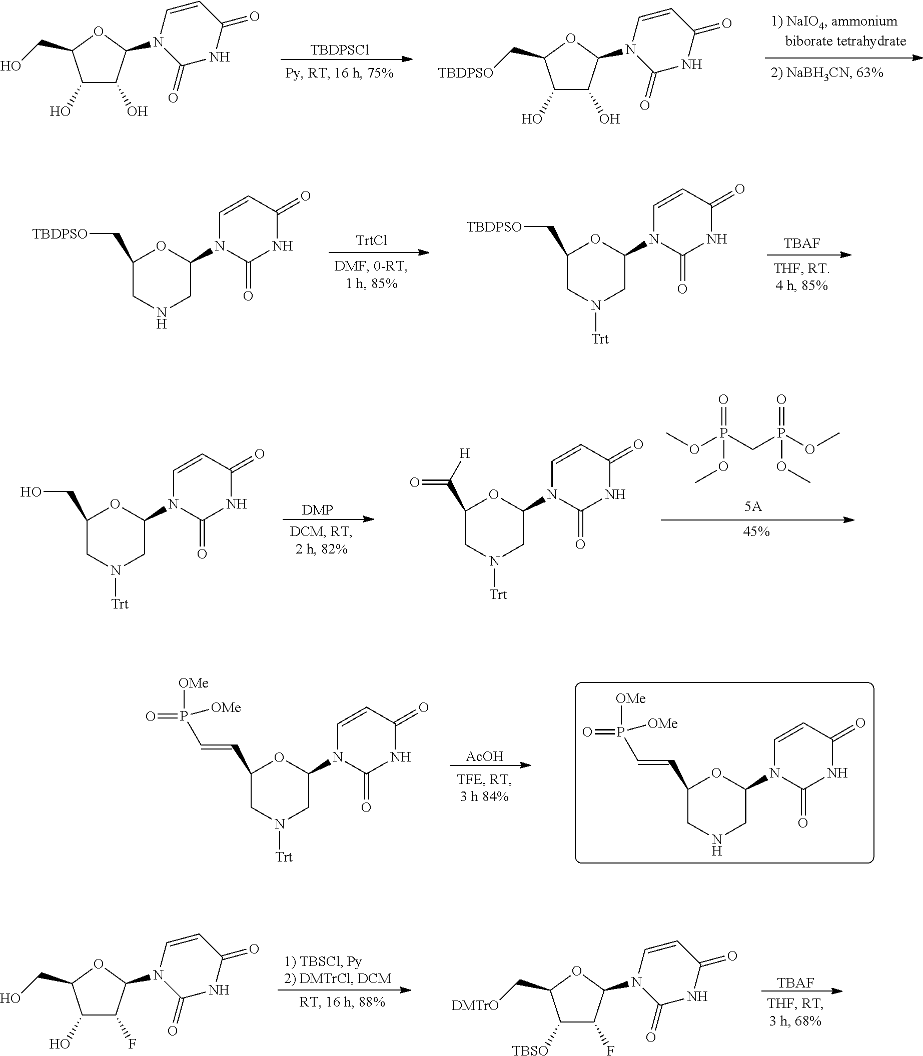

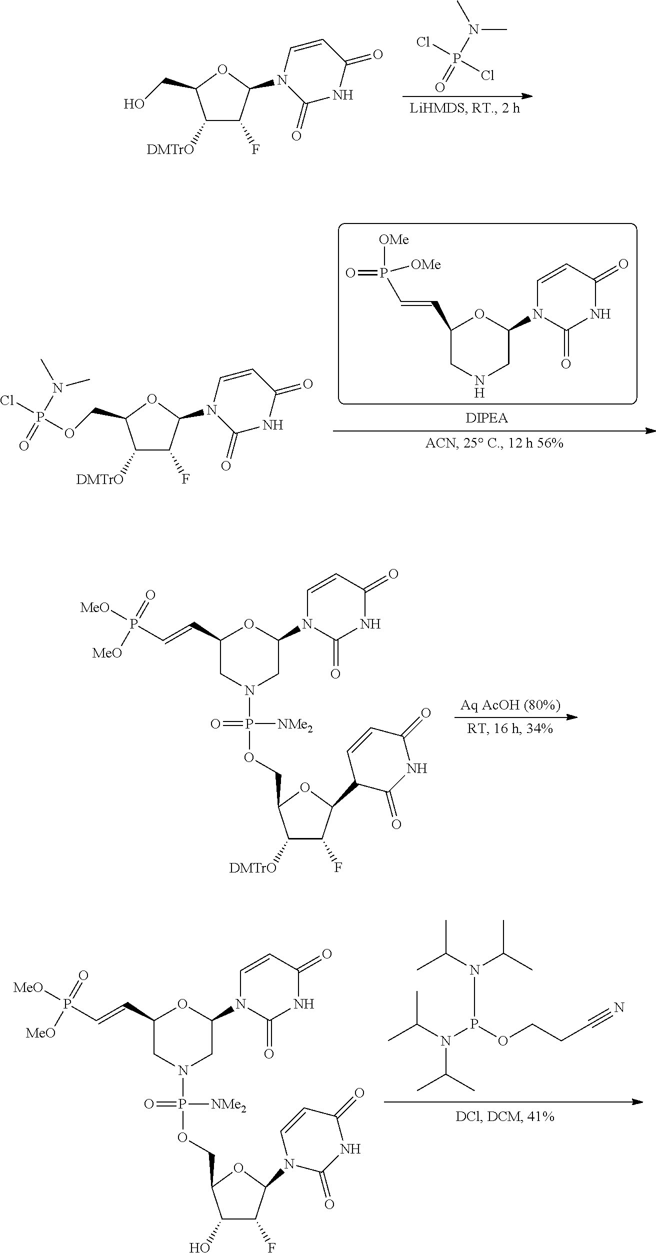

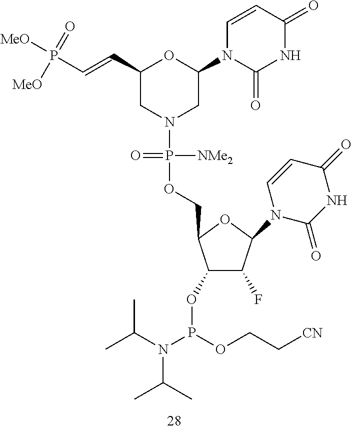 [SMILES: COP(=O)(/C=C/[C@@H]1CN(P(=O)(OC[C@H]2O[C@@H](n3ccc(=O)[nH]c3=O)[C@H](F)[C@@H]2OP(OCCC#N)N(C(C)C)C(C)C)N(C)C)C[C@H](n2ccc(=O)[nH]c2=O)O1)OC]

### Example 22. Preparation of Compound 29

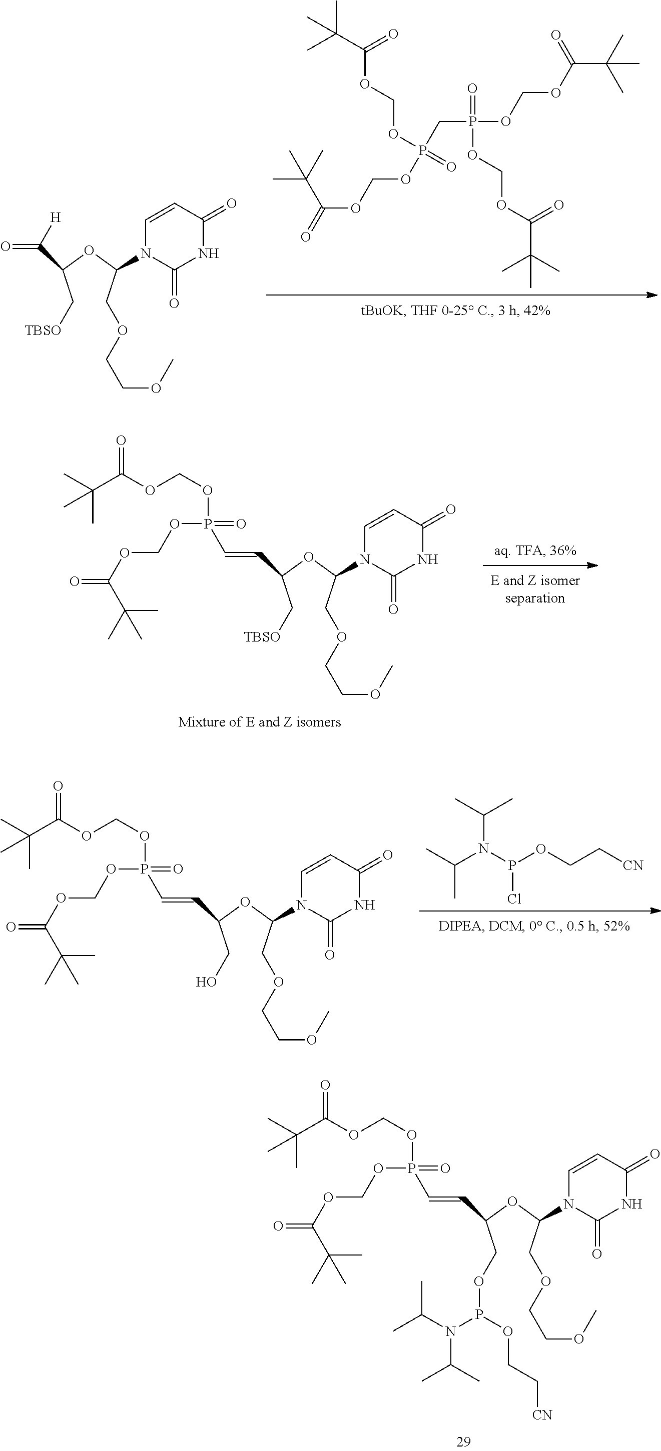

### Example 23. Preparation of Compound 30

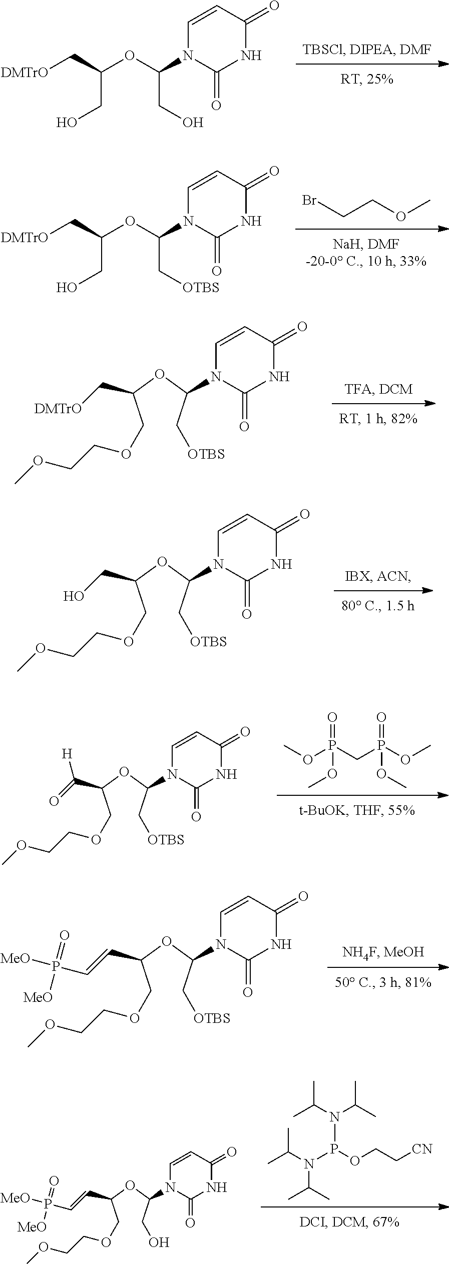

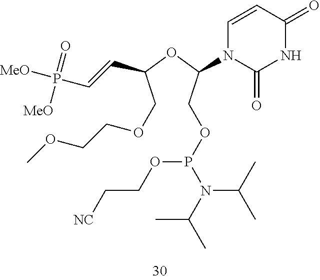 [SMILES: COCCOC[C@@H](/C=C/P(=O)(OC)OC)O[C@H](COP(OCCC#N)N(C(C)C)C(C)C)n1ccc(=O)[nH]c1=O]

### Example 24. Preparation of Compound 31

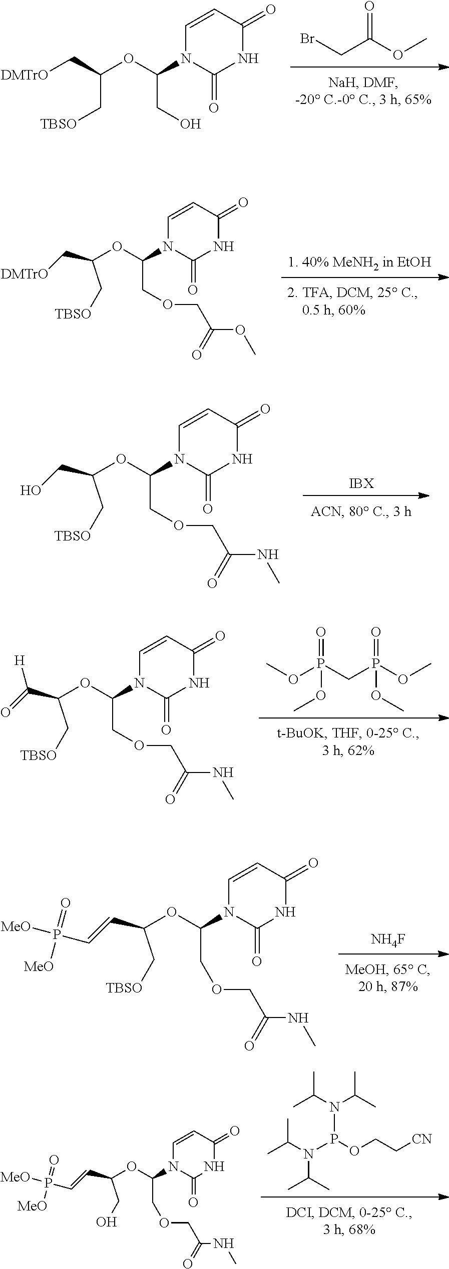

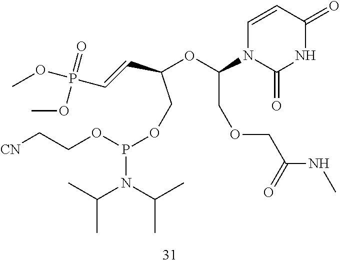

### Example 25. Preparation of Compound 32

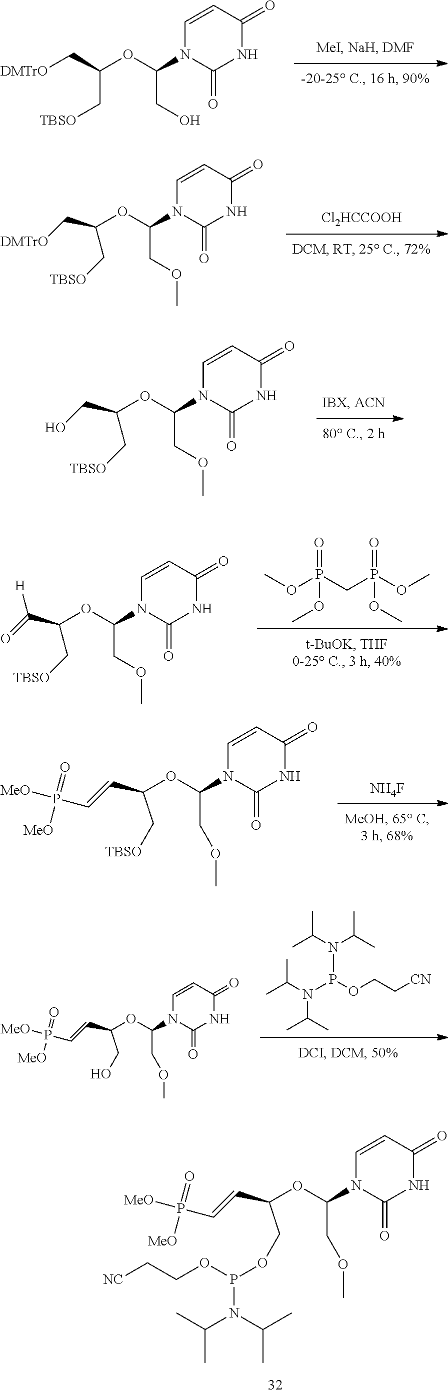

### Example 26. Synthetic Protocol for Preparation of Compound 26

### Example 27. Analytical Data

³¹P decoupled ¹HNMR, ³¹P NMR and MS (ESI) data for various of the compounds described herein are presented below:

| Chemical structure | Analytical (NMR and MS) data |
|---|---|
| 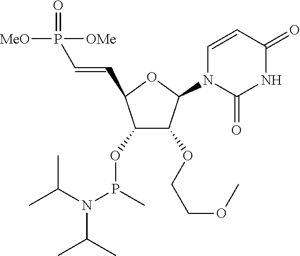 | ³¹P decoupled ¹H NMR (400 MHz, CD₃CN): δ 7.39- 7.35(dd, J = 7.6 Hz, 1H), δ6.77(m, 1H), 6.0(m, 1H), 5.82-5.78 (dd, J = 3.6, 14 Hz, 1H), 5.66-5.63(dd, J = 4, 8 Hz, 1H), 4.52- 4.46(m, 1H), 4.16-4.01(m, 2H), 3.73-3.47(br, 12H), 3.28(s, 3H), 1.26(d, J = 6.4 Hz, 3H), 1.18-1.16(d, J = 6.4 Hz, 6H), 1.09-1.08(d, J = 6.4 Hz, 6H) ³¹P NMR (400 MHz, CD₃CN) δ125.7, 125.5, 19.09, 18.95 MS (ESI) calculated for C₂₄H₄₂N₄O₁₀P₂ (M + H)+ m/z = 652.2, found 652.2 |
| 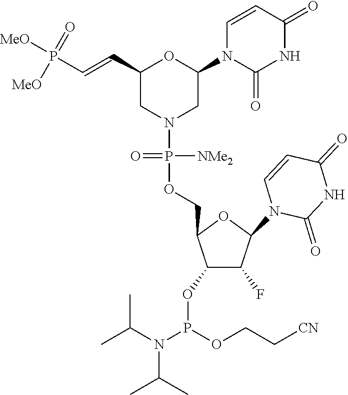 [SMILES: COP(=O)(/C=C/[C@@H]1CN(P(=O)(OC[C@H]2O[C@@H](n3ccc(=O)[nH]c3=O)[C@H](F)[C@@H]2OP(OCCC#N)N(C(C)C)C(C)C)N(C)C)C[C@H](n2ccc(=O)[nH]c2=O)O1)OC] | ³¹P decoupled ¹H NMR (400 MHz, CD₃CN): δ7.70- 7.61(m, 1H), δ7.55-7.48(m, 1H), 7.08-6.73(br, 1H), 6.01(br, 1H), 5.69(br, 4H), 5.3 (m, 1H), 4.78-4.49(m, 2H), 4.11(br, 3H), 3.90-3.48(br, 14H), 2.83-2.59(Br, 10H), 1.35- 1.20(m, 12H) ³¹P NMR (400 MHz, CD₃CN) δ150.75, 150.71, 150.05, 149.99, 20.54, 20.44, 15.39, 15.29 MS (ESI) calculated for C₃₂H₅₀FN₈O₁₃P₃ (M − H)⁻ m/z = 865.2, found 865.2 |
| 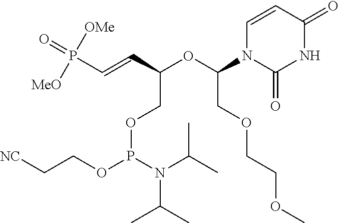 [SMILES: COCCOC[C@@H](O[C@H](/C=C/P(=O)(OC)OC)COP(OCCC#N)N(C(C)C)C(C)C)n1ccc(=O)[nH]c1=O] | ³¹P decoupled ¹H NMR (400 MHz, CD₃CN): δ 7.42- 7.45(dd, J = 8 Hz, 1H), δ 6.42-6.47(dd, J = 17.2 Hz, 1H), 5.97- 5.84(m, 2H), 5.54-5.52(d, J = 8 Hz, 1H), 4.27-4.23(m, 1H), 3.75-3.50(br, 12H), 3.36-3.30(br, 2H), 3.18(s, 3H), 2.60- 2.57(m, 2H), 1.10-1.09(d, 12H) ³¹P NMR (400 MHz, CD₃CN) δ148.6, 148.4, 19.24, 19.22 MS (ESI) calculated for C₂₄H₄₂N₄O₁₀P₂ (M − H)⁻ m/z = 607.2, found 607.2 |
| 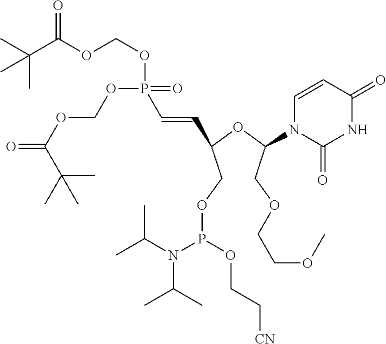 [SMILES: COCCOC[C@@H](O[C@H](/C=C/P(=O)(OCOC(=O)C(C)(C)C)OCOC(=O)C(C)(C)C)COP(OCCC#N)N(C(C)C)C(C)C)n1ccc(=O)[nH]c1=O] | ³¹P decoupled ¹H NMR (400 MHz, CD₃CN): δ 7.55 (dd, J = 8 Hz, 1H), δ 6.58-6.69 (m, J = 17.2 Hz, 1H), 6.11-6.02 (m, 2H), 5.66-5.47 (m, 5H), 4.36-4.38 (m, 1H), 3.88-3.60 (m, 9H), 3.46-3.44 (br, 2H), 3.29 (s, 3H), 2.69 (dt, J = 1.6 Hz, 6 Hz, 2H), 1.20 (m, 30H) ³¹P NMR (400 MHz, CD₃CN) δ148.69, 148.49, 16.79 MS (ESI) calculated for C₃₄H₅₈N₄O₁₄P₂ (M − H)⁻ m/z = 807.3, found 807.3 |
| 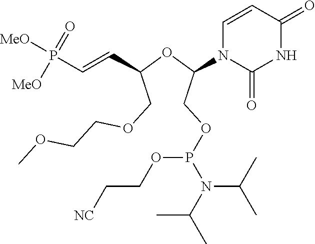 [SMILES: COCCOC[C@@H](/C=C/P(=O)(OC)OC)O[C@H](COP(OCCC#N)N(C(C)C)C(C)C)n1ccc(=O)[nH]c1=O] | ³¹P decoupled ¹H NMR (400 MHz, CD₃CN): δ 7.55- 7.52(dd, J = 8 Hz, 1H), δ 6.58-6.52(dd, J = 17.2 Hz, 1H), 6.08- 5.94(m, 2H), 5.67-5.64(d, J = 8 Hz, 1H), 4.39-4.35(m, 1H), 3.95-3.79(br, 4H), 3.75-3.58 (br, 12H), 3.57-3.49(br, 2H), 3.33(br, 3H), 2.68-2.65(t, 2H), 1.18-1.13(m, 12H) ³¹P NMR (400 MHz, CD₃CN) δ149.05, 148.82, 19.41, 19.38 MS (ESI) calculated for C₂₄H₄₂N₄O₁₀P₂ (M − H)⁻ m/z = 607.2, found 607.2 |
| 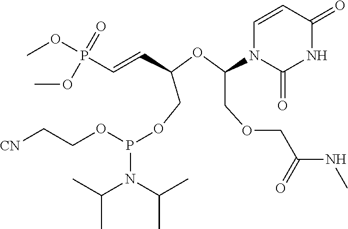 | ¹H NMR (400 MHz, CD₃CN): δ7.55 (d, J = 8 Hz, 1H), 6.83 (br, 1H) δ6.57 (ddd, J = 5.2, 17.2, 39.2 Hz, 1H), 6.12-5.95 (m, 2H), 5.68(d, J = 8 Hz, 1H), 4.4 (br, 1H), 3.96 (s, 2H), 3.88-3.52 (br, 14H), 2.35-2.68 (br, 5H), 1.20 (d, J = 6.8 Hz, 12H) ³¹P NMR (400 MHz, CD₃CN) δ148.6, 148.4, 19.28 MS (ESI) calculated for chemical formula: C₂₄H₄₁N₅O₁₀P₂ (M − H)⁻ m/z = 620.2, found 620.2 |
| 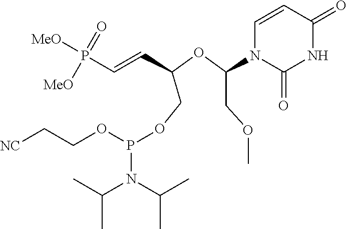 [SMILES: COC[C@@H](O[C@H](/C=C/P(=O)(OC)OC)COP(OCCC#N)N(C(C)C)C(C)C)n1ccc(=O)[nH]c1=O] | ¹H NMR (400 MHz, CD₃CN): δ9.02, (br, 1H), δ7.51 (d, J = 8 Hz, 1H), 6.53 (br, 1H) δ6.06 (br, 2H), 5.68(d, J = 8 Hz, 1H), 4.37 (m, 1H), 3.87-3.7 (m, 4H), 3.69-3.63 (m, 10H), 3.35 (s, 3H), 2.70 (m, 2H), 1.22 (m, 12H) ³¹P NMR (400 MHz, CD₃CN) δ148.7, 148.4, 19.19, 19.15 MS (ESI) calculated for chemical formula: C₂₂H₃₈N₄O₉P₂ (M − H)⁻ m/z = 563.2, found 563.2 |
| 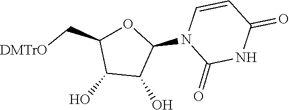 | ¹H NMR: 400 MHz, DMSO-d₆ δ ppm 11.35 (s, 1H), 7.72 (d, J = 8.0 Hz, 1H), 7.36- 7.41 (m, 2H), 7.32 (t, J = 7.6 Hz, 2H), 7.20-7.28 (m, 5H), 6.90 (d, J = 8.6 Hz, 4H), 5.48 (d, J = 4.8 Hz, 1H), 5.31 (d, J = 8.0 Hz, 1H), 5.14 (d, J = 5.6 Hz, 1H), 4.09 (q, J = 5.4 Hz, 2H), 3.92-3.99 (m, 1H), 3.70-3.78 (m, 6H), 3.18-3.30 (m, 2H). |
| 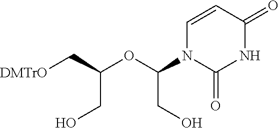 | ¹H NMR: 400 MHz, DMSO-d₆ δ ppm 11.35 (s, 1H), 7.65 (d, J = 8.0 Hz, 1H), 7.28- 7.33 (m, 4H), 7.17-7.21 (m, 5H), 6.86 (d, J = 8.0 Hz, 4H), 5.83 (t, J = 6.4 Hz, 1H), 5.53 (d, J = 8.0 Hz, 1H), 5.13 (t, J = 8.0 Hz, 1H), 4.75 (t, J = 5.2 Hz, 1H), 3.76 (s, 6H), 3.68-3.73 (m, 1H), 3.60-3.64 (m, 2H), 3.42 (t, J = 5.2 Hz, 2H), 2.96-3.02 (m, 2H) |
| 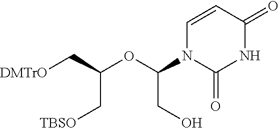 | ¹H NMR: 400 MHz, DMSO-d₆ δ ppm 11.35 (s, 1H), 7.65 (d, J = 8.0 Hz, 1H), 7.24- 7.33 (m, 4H), 7.14-7.22 (m, 5H), 6.82-6.88 (m, 4H), 5.83 (t, J = 5.8 Hz, 1H), 5.47-5.57 (m, 1H), 5.11 (t, J = 5.8 Hz, 1H), 3.72 (d, J = 0.8 Hz, 6H), 3.57-3.67 (m, 4H), 3.46-3.55 (m, 1H), 2.94-3.00 (m, 2H), 0.72-0.78 (m, 9H), −0.04 (d, J = 8.2 Hz, 5H) |
| 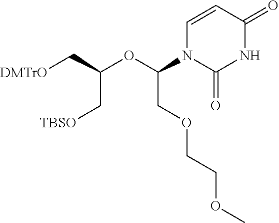 | ¹H NMR: 400 MHz, DMSO-d₆ δ ppm 11.40 (d, J = 1.8 Hz, 1H), 7.68 (d, J = 8.2 Hz, 1H), 7.24-7.33 (m, 5H), 7.14-7.22 (m, 5H), 6.81-6.88 (m, 4H), 5.96-6.02 (m, 1H), 5.55 (dd, J = 8.0, 2.0 Hz, 1H), 3.73 (d, J = 0.8 Hz, 6H), 3.70 (dd, J = 6.0., 2.2 Hz, 2H), 3.62-3.66 (m, 2H), 3.53-3.57 (m, 2H), 3.50- 3.53 (m, 1H), 3.38-3.42 (m, 2H), 3.20-3.22 (m, 3H), 2.94-3.00 (m, 2H), 0.72-0.82 (m, 9H), −0.04 (d, J = 7.4 Hz, 5H) |
| 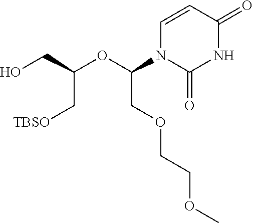 [SMILES: COCCOC[C@@H](O[C@H](CO)CO[Si](C)(C)C(C)(C)C)n1ccc(=O)[nH]c1=O] | ¹H NMR: 400 MHz, DMSO-d₆ δ ppm 11.87 (s, 1H), 8.17 (d, J = 8.2 Hz, 1H), 7.75- 7.87 (m, 4H), 7.63-7.74 (m, 5H), 7.36 (dd, J = 9.0, 2.6 Hz, 4H), 6.34 (t, J = 5.8 Hz, 1H), 6.05 (d, J = 8.0 Hz, 1H), 5.62 (t, J = 5.8 Hz, 1H), 4.24 (s, 6H), 4.09-4.20 (m, 4H), 4.02 (d, J = 4.6 Hz, 1H), 3.83 (s, 1H), 2.95- 3.09 (m, 4H), 1.28 (s, 9H), 0.48 (d, J = 8.2 Hz, 6H). |
| 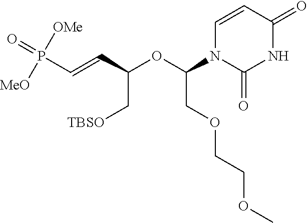 [SMILES: COCCOC[C@@H](O[C@H](/C=C/P(=O)(OC)OC)CO[Si](C)(C)C(C)(C)C)n1ccc(=O)[nH]c1=O] | ¹H NMR: 400 MHz, CDCl₃ δ ppm 9.19 (s, 1H), 7.43 (d, J = 8.16 Hz, 1H), 6.51- 6.67 (m, 1H), 6.13 (t, J = 4.78 Hz, 1H), 5.82-5.95 (m, 1H), 5.72 (dd, J = 8.04, 1.88 Hz, 1H), 4.20 (s, 1H), 3.63-3.78 (m, 12H), 3.45-3.54 (m, 2H), 3.31-3.37 (m, 3H), 0.82-0.94 (m, 9H), 0.00-0.10 (m, 6H). |
| 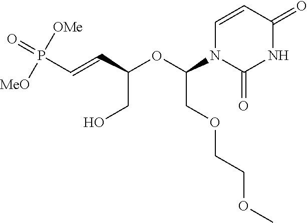 [SMILES: COCCOC[C@@H](O[C@H](/C=C/P(=O)(OC)OC)CO)n1ccc(=O)[nH]c1=O] | ¹H NMR: 400 MHz, DMSO-d₆ δ ppm 11.29 (s, 1H) 7.74 (d, J = 8.0 Hz, 1H) 6.42-6.62 (m, 1H) 5.90-5.98 (m, 2H) 5.62 (dd, J = 8.0, 1.2 Hz, 1H) 5.01 (t, J = 5.6 Hz, 1H) 4.18 (d, J = 1.6 Hz, 1H) 4.10 (q, J = 5.2 Hz, 1H) 3.70-3.76 (m, 2H) 3.50-3.58 (m, 8H) 3.43-3.49 (m, 1H) 3.36-3.41 (m, 2H) 3.21 (s, 3H) 3.17 (d, J = 5.2 Hz, 2H) 1.91 (s, 1H) |
| 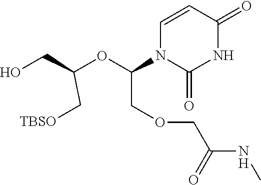 [SMILES: CNC(=O)COC[C@@H](O[C@H](CO)CO[Si](C)(C)C(C)(C)C)n1ccc(=O)[nH]c1=O] | ¹H NMR: 400 MHz, DMSO-d₆ δ 11.30 (d, J = 1.8 Hz, 1H), 7.67 (d, J = 8.0 Hz, 1H), 7.61 (br d, J = 4.3 Hz, 1H), 6.03 (t, J = 5.6 Hz, 1H), 5.61 (dd, J = 2.0, 8.0 Hz, 1H), 4.66 (br s, 1H), 3.90 (s, 2H), 3.73-3.57 (m, 4H), 3.49-3.43 (m, 1H), 3.34-3.25 (m, 2H), 2.60 (d, J = 4.8 Hz, 3H), 0.87 (s, 9H), 0.05 (s, 6H) |
| 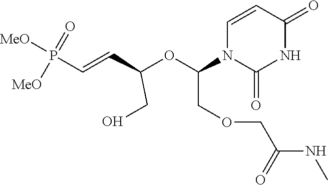 [SMILES: CNC(=O)COC[C@@H](O[C@H](/C=C/P(=O)(OC)OC)CO)n1ccc(=O)[nH]c1=O] | ¹HNMR: 400 MHz CD₃CN δ 7.52 (d, J = 8.2 Hz, 1H), 6.98 (br s, 1H), 6.61-6.47 (m, 1H), 6.06-5.86 (m, 2H), 5.65 (d, J = 8.2 Hz, 1H), 4.28-4.20 (m, 1H), 3.96 (s, 2H), 3.80-3.51 (m, 10H), 2.73-2.67 (m, 4H) ³¹PNMR: 162 MHz CD₃CN, δ 19.71 (s, 1P) |
| 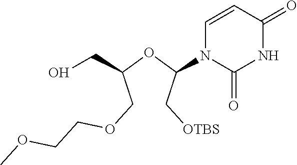 [SMILES: COCCOC[C@@H](CO)O[C@H](CO[Si](C)(C)C(C)(C)C)n1ccc(=O)[nH]c1=O] | ¹H NMR: 400 MHz, DMSO d₆ 11.23 (s, 1H), 7.63 (d, J = 8.02 Hz, 1H), 5.85 (dd, J = 6.56, 4.98 Hz, 1H), 5.60 (dd, J = 7.92, 1.66 Hz, 1H), 4.65 (t, J = 5.58 Hz, 1H), 3.63-3.84 (m, 2H), 3.40-3.60 (m, 7H), 3.26 (s, 3H), 0.81 (s, 9H), 0.01 (d, J = 6.66 Hz, 5H). |
| 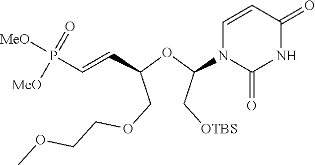 [SMILES: COCCOC[C@@H](/C=C/P(=O)(OC)OC)O[C@H](CO[Si](C)(C)C(C)(C)C)n1ccc(=O)[nH]c1=O] | ¹H NMR: 400 MHz, DMSO d₆ 7.72 (d, J = 8.22 Hz, 1H), 6.38-6.61 (m, 1H), 5.76- 6.09 (m, 2H), 5.61 (dd, J = 8.02, 2.16 Hz, 1H), 4.29- 4.43 (m, 1H), 3.66-3.92 (m, 2H), 3.41-3.63 (m, 12H), 3.17-3.26 (m, 3H), 0.73-0.87 (m, 10H), 0.01 (d, J = 7.04 Hz, 7H). |
| 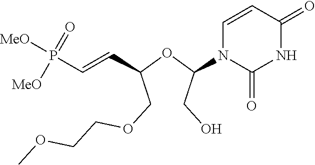 [SMILES: COCCOC[C@@H](/C=C/P(=O)(OC)OC)O[C@H](CO)n1ccc(=O)[nH]c1=O] | ¹H NMR: 400 MHz, DMSO d₆ 11.26 (d, J = 1.52 Hz, 1H), 7.71 (d, J = 8.04 Hz, 1H), 6.43-6.58 (m, 1H), 5.88-6.06 (m, 1H), 5.75-5.85 (m, 1H), 5.61 (dd, J = 7.96, 2.08 Hz, 1H), 5.14 (t, J = 6.02 Hz, 1H), 4.34 (br d, J = 1.52 Hz, 1H), 3.42-3.70 (m, 14H), 3.26 (s, 3H) |
| 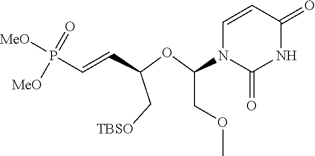 [SMILES: COC[C@@H](O[C@H](/C=C/P(=O)(OC)OC)CO[Si](C)(C)C(C)(C)C)n1ccc(=O)[nH]c1=O] | ¹H NMR: 400 MHz, DMSO-d₆ 11.31 (s, 1H), 7.72-7.77 (m, 1H), 6.45-6.56 (m, 1H), 5.91-6.03 (m, 2H), 5.62 (dd, J = 8.40, 2.40 Hz, 1H), 4.24 (br s, 1H), 3.72-3.76 (m, 1H), 3.61-3.69 (m, 3H), 3.52-3.59 (m, 6H), 3.26 (s, 3H), 0.87 (s, 9H), 0.06 (d, J = 1.60 Hz, 6H). ³¹P NMR: 162 MHz, DMSO-d₆ 19.93 (s, 1P) |
| 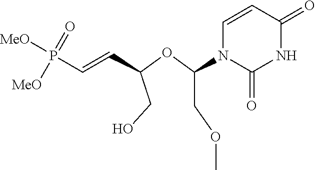 [SMILES: COC[C@@H](O[C@H](/C=C/P(=O)(OC)OC)CO)n1ccc(=O)[nH]c1=O] | ¹H NMR: 400 MHz, DMSO-d₆ 11.31 (s, 1H), 7.74 (d, J = 8.00 Hz, 1H), 6.47-6.58 (m, 1H), 5.87-5.99 (m, 2H), 5.63 (d, J = 8.4 Hz, 1H), 5.04 (s, 1H), 4.10-4.19 (m, 2H), 3.66 (d, J = 6.00 Hz, 2H), 3.56 (dd, J = 11.2, 4.40 Hz, 6H), 3.43-3.48 (m, 1H), 3.28 (s, 3H), 3.17-3.18 (m, 2H). ³¹P NMR: 162 MHz, DMSO-d₆ 20.04 (s, 1P). |

### MOLECULAR BIOLOGY EXAMPLES

### Example 1. Sequences

Tables 1, 3, 5, 6, and 7 illustrate target sequences described herein. Tables 2, 4, 8, and 9 illustrate polynucleic acid molecule sequences described herein.

TABLE 1
KRAS Target Sequences

| | sequence | | |
|---|---|---|---|
| | position in | | SEQ ID |
| Id # | NM_033360.2 | target site in NM_033360.2 | NO: |
| 182 | 182-200 | AAAUGACUGAAUAUAAACUUGUG | 1 |
| 183 | 183-201 | AAUGACUGAAUAUAAACUUGUGG | 2 |
| 197 | 197-215 | AACUUGUGGUAGUUGGAGCUGGU | 3 |
| 224 | 224-242 | UAGGCAAGAGUGCCUUGACGAUA | 4 |
| 226 | 226-244 | GGCAAGAGUGCCUUGACGAUACA | 5 |
| 227 | 227-245 | GCAAGAGUGCCUUGACGAUACAG | 6 |
| 228 | 228-246 | CAAGAGUGCCUUGACGAUACAGC | 7 |
| 232 | 232-250 | AGUGCCUUGACGAUACAGCUAAU | 8 |
| 233 | 233-251 | GUGCCUUGACGAUACAGCUAAUU | 9 |
| 236 | 236-254 | CCUUGACGAUACAGCUAAUUCAG | 10 |
| 237 | 237-255 | CUUGACGAUACAGCUAAUUCAGA | 11 |
| 245 | 245-263 | UACAGCUAAUUCAGAAUCAUUUU | 12 |
| 266 | 266-284 | UUGUGGACGAAUAUGAUCCAACA | 13 |
| 269 | 269-287 | UGGACGAAUAUGAUCCAACAAUA | 14 |
| 270 | 270-288 | GGACGAAUAUGAUCCAACAAUAG | 15 |

TABLE 2
KRAS siRNA sequences

| | sequence | | | | |
|---|---|---|---|---|---|
| | position in | sense strand | SEQ ID | antisense strand | SEQ ID |
| Id # | NM_033360.2 | sequence (5′-3′) | NO: | sequence (5′-3′) | NO: |
| 182 | 182-200 | AUGACUGAAUAUAAACU | 16 | CAAGUUUAUAUUCAG | 17 |
| | | UGTT | | UCAUTT | |
| 183 | 183-201 | UGACUGAAUAUAAACUU | 18 | ACAAGUUUAUAUUCA | 19 |
| | | GUTT | | GUCATT | |
| 197 | 197-215 | CUUGUGGUAGUUGGAGC | 20 | CAGCUCCAACUACCA | 21 |
| | | UGTT | | CAAGTT | |
| 224 | 224-242 | GGCAAGAGUGCCUUGAC | 22 | UCGUCAAGGCACUCU | 23 |
| | | GATT | | UGCCTT | |
| 226 | 226-244 | CAAGAGUGCCUUGACGA | 24 | UAUCGUCAAGGCACU | 25 |
| | | UATT | | CUUGTT | |
| 227 | 227-245 | AAGAGUGCCUUGACGAU | 26 | GUAUCGUCAAGGCAC | 27 |
| | | ACTT | | UCUUTT | |
| 228 | 228-246 | AGAGUGCCUUGACGAUA | 28 | UGUAUCGUCAAGGCA | 29 |
| | | CATT | | CUCUTT | |
| 232 | 232-250 | UGCCUUGACGAUACAGC | 30 | UAGCUGUAUCGUCAA | 31 |
| | | UATT | | GGCATT | |
| 233 | 233-251 | GCCUUGACGAUACAGCU | 32 | UUAGCUGUAUCGUCA | 33 |
| | | AATT | | AGGCTT | |
| 236 | 236-254 | UUGACGAUACAGCUAAU | 34 | GAAUUAGCUGUAUCG | 35 |
| | | UCTT | | UCAATT | |
| 237 | 237-255 | UGACGAUACAGCUAAUU | 36 | UGAAUUAGCUGUAUC | 37 |
| | | CATT | | GUCATT | |
| 245 | 245-263 | CAGCUAAUUCAGAAUCA | 38 | AAUGAUUCUGAAUUA | 39 |
| | | UUTT | | GCUGTT | |
| 266 | 266-284 | GUGGACGAAUAUGAUCC | 40 | UUGGAUCAUAUUCGU | 41 |
| | | AATT | | CCACTT | |
| 269 | 269-287 | GACGAAUAUGAUCCAAC | 42 | UUGUUGGAUCAUAUU | 43 |
| | | AATT | | CGUCTT | |
| 270 | 270-288 | ACGAAUAUGAUCCAACA | 44 | AUUGUUGGAUCAUAU | 45 |
| | | AUTT | | UCGUTT | |

TABLE 3
EGFR Target Sequences

| hs | 19mer | | SEQ |
|---|---|---|---|
| Id | pos. in | sequence of total 23mer | ID |
| # | NM_005228.3 | target site in NM_005228.3 | NO: |
| 68 | 68-86 | GGCGGCCGGAGUCCCGAGCUAGC | 46 |
| 71 | 71-89 | GGCCGGAGUCCCGAGCUAGCCCC | 47 |
| 72 | 72-90 | GCCGGAGUCCCGAGCUAGCCCCG | 48 |
| 73 | 73-91 | CCGGAGUCCCGAGCUAGCCCCGG | 49 |
| 74 | 74-92 | CGGAGUCCCGAGCUAGCCCCGGC | 50 |
| 75 | 75-93 | GGAGUCCCGAGCUAGCCCCGGCG | 51 |
| 76 | 76-94 | GAGUCCCGAGCUAGCCCCGGCGG | 52 |
| 78 | 78-96 | GUCCCGAGCUAGCCCCGGCGGCC | 53 |
| 114 | 114-132 | CCGGACGACAGGCCACCUCGUCG | 54 |
| 115 | 115-133 | CGGACGACAGGCCACCUCGUCGG | 55 |
| 116 | 116-134 | GGACGACAGGCCACCUCGUCGGC | 56 |
| 117 | 117-135 | GACGACAGGCCACCUCGUCGGCG | 57 |
| 118 | 118-136 | ACGACAGGCCACCUCGUCGGCGU | 58 |
| 120 | 120-138 | GACAGGCCACCUCGUCGGCGUCC | 59 |
| 121 | 121-139 | ACAGGCCACCUCGUCGGCGUCCG | 60 |
| 122 | 122-140 | CAGGCCACCUCGUCGGCGUCCGC | 61 |
| 123 | 123-141 | AGGCCACCUCGUCGGCGUCCGCC | 62 |
| 124 | 124-142 | GGCCACCUCGUCGGCGUCCGCCC | 63 |
| 125 | 125-143 | GCCACCUCGUCGGCGUCCGCCCG | 64 |
| 126 | 126-144 | CCACCUCGUCGGCGUCCGCCCGA | 65 |
| 127 | 127-145 | CACCUCGUCGGCGUCCGCCCGAG | 66 |
| 128 | 128-146 | ACCUCGUCGGCGUCCGCCCGAGU | 67 |
| 129 | 129-147 | CCUCGUCGGCGUCCGCCCGAGUC | 68 |
| 130 | 130-148 | CUCGUCGGCGUCCGCCCGAGUCC | 69 |
| 131 | 131-149 | UCGUCGGCGUCCGCCCGAGUCCC | 70 |
| 132 | 132-150 | CGUCGGCGUCCGCCCGAGUCCCC | 71 |
| 135 | 135-153 | CGGCGUCCGCCCGAGUCCCCGCC | 72 |
| 136 | 136-154 | GGCGUCCGCCCGAGUCCCCGCCU | 73 |
| 141 | 141-159 | CCGCCCGAGUCCCCGCCUCGCCG | 74 |
| 164 | 164-182 | CCAACGCCACAACCACCGCGCAC | 75 |
| 165 | 165-183 | CAACGCCACAACCACCGCGCACG | 76 |
| 166 | 166-184 | AACGCCACAACCACCGCGCACGG | 77 |
| 168 | 168-186 | CGCCACAACCACCGCGCACGGCC | 78 |
| 169 | 169-187 | GCCACAACCACCGCGCACGGCCC | 79 |
| 170 | 170-188 | CCACAACCACCGCGCACGGCCCC | 80 |
| 247 | 247-265 | CGAUGCGACCCUCCGGGACGGCC | 81 |
| 248 | 248-266 | GAUGCGACCCUCCGGGACGGCCG | 82 |
| 249 | 249-267 | AUGCGACCCUCCGGGACGGCCGG | 83 |
| 251 | 251-269 | GCGACCCUCCGGGACGGCCGGGG | 84 |
| 252 | 252-270 | CGACCCUCCGGGACGGCCGGGGC | 85 |
| 254 | 254-272 | ACCCUCCGGGACGGCCGGGGCAG | 86 |
| 329 | 329-347 | AAAGAAAGUUUGCCAAGGCACGA | 87 |
| 330 | 330-348 | AAGAAAGUUUGCCAAGGCACGAG | 88 |
| 332 | 332-350 | GAAAGUUUGCCAAGGCACGAGUA | 89 |
| 333 | 333-351 | AAAGUUUGCCAAGGCACGAGUAA | 90 |
| 334 | 334-352 | AAGUUUGCCAAGGCACGAGUAAC | 91 |
| 335 | 335-353 | AGUUUGCCAAGGCACGAGUAACA | 92 |
| 336 | 336-354 | GUUUGCCAAGGCACGAGUAACAA | 93 |
| 337 | 337-355 | UUUGCCAAGGCACGAGUAACAAG | 94 |
| 338 | 338-356 | UUGCCAAGGCACGAGUAACAAGC | 95 |
| 361 | 361-379 | UCACGCAGUUGGGCACUUUUGAA | 96 |
| 362 | 362-380 | CACGCAGUUGGGCACUUUUGAAG | 97 |
| 363 | 363-381 | ACGCAGUUGGGCACUUUUGAAGA | 98 |
| 364 | 364-382 | CGCAGUUGGGCACUUUUGAAGAU | 99 |
| 365 | 365-383 | GCAGUUGGGCACUUUUGAAGAUC | 100 |
| 366 | 366-384 | CAGUUGGGCACUUUUGAAGAUCA | 101 |
| 367 | 367-385 | AGUUGGGCACUUUUGAAGAUCAU | 102 |
| 368 | 368-386 | GUUGGGCACUUUUGAAGAUCAUU | 103 |
| 369 | 369-387 | UUGGGCACUUUUGAAGAUCAUUU | 104 |
| 377 | 377-395 | UUUUGAAGAUCAUUUUCUCAGCC | 105 |
| 379 | 379-397 | UUGAAGAUCAUUUUCUCAGCCUC | 106 |
| 380 | 380-398 | UGAAGAUCAUUUUCUCAGCCUCC | 107 |
| 385 | 385-403 | AUCAUUUUCUCAGCCUCCAGAGG | 108 |
| 394 | 394-412 | UCAGCCUCCAGAGGAUGUUCAAU | 109 |
| 396 | 396-414 | AGCCUCCAGAGGAUGUUCAAUAA | 110 |
| 397 | 397-415 | GCCUCCAGAGGAUGUUCAAUAAC | 111 |
| 401 | 401-419 | CCAGAGGAUGUUCAAUAACUGUG | 112 |
| 403 | 403-421 | AGAGGAUGUUCAAUAACUGUGAG | 113 |
| 407 | 407-425 | GAUGUUCAAUAACUGUGAGGUGG | 114 |
| 409 | 409-427 | UGUUCAAUAACUGUGAGGUGGUC | 115 |
| 410 | 410-428 | GUUCAAUAACUGUGAGGUGGUCC | 116 |
| 411 | 411-429 | UUCAAUAACUGUGAGGUGGUCCU | 117 |
| 412 | 412-430 | UCAAUAACUGUGAGGUGGUCCUU | 118 |
| 413 | 413-431 | CAAUAACUGUGAGGUGGUCCUUG | 119 |
| 414 | 414-432 | AAUAACUGUGAGGUGGUCCUUGG | 120 |
| 416 | 416-434 | UAACUGUGAGGUGGUCCUUGGGA | 121 |
| 418 | 418-436 | ACUGUGAGGUGGUCCUUGGGAAU | 122 |
| 419 | 419-437 | CUGUGAGGUGGUCCUUGGGAAUU | 123 |
| 425 | 425-443 | GGUGGUCCUUGGGAAUUUGGAAA | 124 |
| 431 | 431-449 | CCUUGGGAAUUUGGAAAUUACCU | 125 |
| 432 | 432-450 | CUUGGGAAUUUGGAAAUUACCUA | 126 |
| 433 | 433-451 | UUGGGAAUUUGGAAAUUACCUAU | 127 |
| 434 | 434-452 | UGGGAAUUUGGAAAUUACCUAUG | 128 |
| 458 | 458-476 | GCAGAGGAAUUAUGAUCUUUCCU | 129 |
| 459 | 459-477 | CAGAGGAAUUAUGAUCUUUCCUU | 130 |
| 463 | 463-481 | GGAAUUAUGAUCUUUCCUUCUUA | 131 |
| 464 | 464-482 | GAAUUAUGAUCUUUCCUUCUUAA | 132 |
| 466 | 466-484 | AUUAUGAUCUUUCCUUCUUAAAG | 133 |
| 468 | 468-486 | UAUGAUCUUUCCUUCUUAAAGAC | 134 |
| 471 | 471-489 | GAUCUUUCCUUCUUAAAGACCAU | 135 |
| 476 | 476-494 | UUCCUUCUUAAAGACCAUCCAGG | 136 |
| 477 | 477-495 | UCCUUCUUAAAGACCAUCCAGGA | 137 |
| 479 | 479-497 | CUUCUUAAAGACCAUCCAGGAGG | 138 |
| 481 | 481-499 | UCUUAAAGACCAUCCAGGAGGUG | 139 |
| 482 | 482-500 | CUUAAAGACCAUCCAGGAGGUGG | 140 |
| 492 | 492-510 | AUCCAGGAGGUGGCUGGUUAUGU | 141 |
| 493 | 493-511 | UCCAGGAGGUGGCUGGUUAUGUC | 142 |
| 494 | 494-512 | CCAGGAGGUGGCUGGUUAUGUCC | 143 |
| 495 | 495-513 | CAGGAGGUGGCUGGUUAUGUCCU | 144 |
| 496 | 496-514 | AGGAGGUGGCUGGUUAUGUCCUC | 145 |
| 497 | 497-515 | GGAGGUGGCUGGUUAUGUCCUCA | 146 |
| 499 | 499-517 | AGGUGGCUGGUUAUGUCCUCAUU | 147 |
| 520 | 520-538 | UUGCCCUCAACACAGUGGAGCGA | 148 |
| 542 | 542-560 | AAUUCCUUUGGAAAACCUGCAGA | 149 |
| 543 | 543-561 | AUUCCUUUGGAAAACCUGCAGAU | 150 |
| 550 | 550-568 | UGGAAAACCUGCAGAUCAUCAGA | 151 |
| 551 | 551-569 | GGAAAACCUGCAGAUCAUCAGAG | 152 |
| 553 | 553-571 | AAAACCUGCAGAUCAUCAGAGGA | 153 |
| 556 | 556-574 | ACCUGCAGAUCAUCAGAGGAAAU | 154 |
| 586 | 586-604 | ACGAAAAUUCCUAUGCCUUAGCA | 155 |
| 587 | 587-605 | CGAAAAUUCCUAUGCCUUAGCAG | 156 |
| 589 | 589-607 | AAAAUUCCUAUGCCUUAGCAGUC | 157 |
| 592 | 592-610 | AUUCCUAUGCCUUAGCAGUCUUA | 158 |
| 593 | 593-611 | UUCCUAUGCCUUAGCAGUCUUAU | 159 |
| 594 | 594-612 | UCCUAUGCCUUAGCAGUCUUAUC | 160 |
| 596 | 596-614 | CUAUGCCUUAGCAGUCUUAUCUA | 161 |
| 597 | 597-615 | UAUGCCUUAGCAGUCUUAUCUAA | 162 |
| 598 | 598-616 | AUGCCUUAGCAGUCUUAUCUAAC | 163 |
| 599 | 599-617 | UGCCUUAGCAGUCUUAUCUAACU | 164 |
| 600 | 600-618 | GCCUUAGCAGUCUUAUCUAACUA | 165 |
| 601 | 601-619 | CCUUAGCAGUCUUAUCUAACUAU | 166 |
| 602 | 602-620 | CUUAGCAGUCUUAUCUAACUAUG | 167 |
| 603 | 603-621 | UUAGCAGUCUUAUCUAACUAUGA | 168 |
| 604 | 604-622 | UAGCAGUCUUAUCUAACUAUGAU | 169 |
| 605 | 605-623 | AGCAGUCUUAUCUAACUAUGAUG | 170 |
| 608 | 608-626 | AGUCUUAUCUAACUAUGAUGCAA | 171 |
| 609 | 609-627 | GUCUUAUCUAACUAUGAUGCAAA | 172 |
| 610 | 610-628 | UCUUAUCUAACUAUGAUGCAAAU | 173 |
| 611 | 611-629 | CUUAUCUAACUAUGAUGCAAAUA | 174 |
| 612 | 612-630 | UUAUCUAACUAUGAUGCAAAUAA | 175 |
| 613 | 613-631 | UAUCUAACUAUGAUGCAAAUAAA | 176 |
| 614 | 614-632 | AUCUAACUAUGAUGCAAAUAAAA | 177 |
| 616 | 616-634 | CUAACUAUGAUGCAAAUAAAACC | 178 |
| 622 | 622-640 | AUGAUGCAAAUAAAACCGGACUG | 179 |
| 623 | 623-641 | UGAUGCAAAUAAAACCGGACUGA | 180 |
| 624 | 624-642 | GAUGCAAAUAAAACCGGACUGAA | 181 |
| 626 | 626-644 | UGCAAAUAAAACCGGACUGAAGG | 182 |
| 627 | 627-645 | GCAAAUAAAACCGGACUGAAGGA | 183 |
| 628 | 628-646 | CAAAUAAAACCGGACUGAAGGAG | 184 |
| 630 | 630-648 | AAUAAAACCGGACUGAAGGAGCU | 185 |
| 631 | 631-649 | AUAAAACCGGACUGAAGGAGCUG | 186 |
| 632 | 632-650 | UAAAACCGGACUGAAGGAGCUGC | 187 |
| 633 | 633-651 | AAAACCGGACUGAAGGAGCUGCC | 188 |
| 644 | 644-662 | GAAGGAGCUGCCCAUGAGAAAUU | 189 |
| 665 | 665-683 | UUUACAGGAAAUCCUGCAUGGCG | 190 |
| 668 | 668-686 | ACAGGAAAUCCUGCAUGGCGCCG | 191 |
| 669 | 669-687 | CAGGAAAUCCUGCAUGGCGCCGU | 192 |
| 670 | 670-688 | AGGAAAUCCUGCAUGGCGCCGUG | 193 |
| 671 | 671-689 | GGAAAUCCUGCAUGGCGCCGUGC | 194 |
| 672 | 672-690 | GAAAUCCUGCAUGGCGCCGUGCG | 195 |
| 674 | 674-692 | AAUCCUGCAUGGCGCCGUGCGGU | 196 |
| 676 | 676-694 | UCCUGCAUGGCGCCGUGCGGUUC | 197 |
| 677 | 677-695 | CCUGCAUGGCGCCGUGCGGUUCA | 198 |
| 678 | 678-696 | CUGCAUGGCGCCGUGCGGUUCAG | 199 |
| 680 | 680-698 | GCAUGGCGCCGUGCGGUUCAGCA | 200 |
| 681 | 681-699 | CAUGGCGCCGUGCGGUUCAGCAA | 201 |
| 682 | 682-700 | AUGGCGCCGUGCGGUUCAGCAAC | 202 |
| 683 | 683-701 | UGGCGCCGUGCGGUUCAGCAACA | 203 |
| 684 | 684-702 | GGCGCCGUGCGGUUCAGCAACAA | 204 |
| 685 | 685-703 | GCGCCGUGCGGUUCAGCAACAAC | 205 |
| 686 | 686-704 | CGCCGUGCGGUUCAGCAACAACC | 206 |
| 688 | 688-706 | CCGUGCGGUUCAGCAACAACCCU | 207 |
| 690 | 690-708 | GUGCGGUUCAGCAACAACCCUGC | 208 |
| 692 | 692-710 | GCGGUUCAGCAACAACCCUGCCC | 209 |
| 698 | 698-716 | CAGCAACAACCCUGCCCUGUGCA | 210 |
| 700 | 700-718 | GCAACAACCCUGCCCUGUGCAAC | 211 |
| 719 | 719-737 | CAACGUGGAGAGCAUCCAGUGGC | 212 |
| 720 | 720-738 | AACGUGGAGAGCAUCCAGUGGCG | 213 |
| 721 | 721-739 | ACGUGGAGAGCAUCCAGUGGCGG | 214 |
| 724 | 724-742 | UGGAGAGCAUCCAGUGGCGGGAC | 215 |
| 725 | 725-743 | GGAGAGCAUCCAGUGGCGGGACA | 216 |
| 726 | 726-744 | GAGAGCAUCCAGUGGCGGGACAU | 217 |
| 733 | 733-751 | UCCAGUGGCGGGACAUAGUCAGC | 218 |
| 734 | 734-752 | CCAGUGGCGGGACAUAGUCAGCA | 219 |
| 736 | 736-754 | AGUGGCGGGACAUAGUCAGCAGU | 220 |
| 737 | 737-755 | GUGGCGGGACAUAGUCAGCAGUG | 221 |
| 763 | 763-781 | UUCUCAGCAACAUGUCGAUGGAC | 222 |
| 765 | 765-783 | CUCAGCAACAUGUCGAUGGACUU | 223 |
| 766 | 766-784 | UCAGCAACAUGUCGAUGGACUUC | 224 |
| 767 | 767-785 | CAGCAACAUGUCGAUGGACUUCC | 225 |
| 769 | 769-787 | GCAACAUGUCGAUGGACUUCCAG | 226 |
| 770 | 770-788 | CAACAUGUCGAUGGACUUCCAGA | 227 |
| 771 | 771-789 | AACAUGUCGAUGGACUUCCAGAA | 228 |
| 772 | 772-790 | ACAUGUCGAUGGACUUCCAGAAC | 229 |
| 775 | 775-793 | UGUCGAUGGACUUCCAGAACCAC | 230 |
| 789 | 789-807 | CAGAACCACCUGGGCAGCUGCCA | 231 |
| 798 | 798-816 | CUGGGCAGCUGCCAAAAGUGUGA | 232 |
| 800 | 800-818 | GGGCAGCUGCCAAAAGUGUGAUC | 233 |
| 805 | 805-823 | GCUGCCAAAAGUGUGAUCCAAGC | 234 |
| 806 | 806-824 | CUGCCAAAAGUGUGAUCCAAGCU | 235 |
| 807 | 807-825 | UGCCAAAAGUGUGAUCCAAGCUG | 236 |
| 810 | 810-828 | CAAAAGUGUGAUCCAAGCUGUCC | 237 |
| 814 | 814-832 | AGUGUGAUCCAAGCUGUCCCAAU | 238 |
| 815 | 815-833 | GUGUGAUCCAAGCUGUCCCAAUG | 239 |
| 817 | 817-835 | GUGAUCCAAGCUGUCCCAAUGGG | 240 |
| 818 | 818-836 | UGAUCCAAGCUGUCCCAAUGGGA | 241 |
| 819 | 819-837 | GAUCCAAGCUGUCCCAAUGGGAG | 242 |
| 820 | 820-838 | AUCCAAGCUGUCCCAAUGGGAGC | 243 |
| 821 | 821-839 | UCCAAGCUGUCCCAAUGGGAGCU | 244 |
| 823 | 823-841 | CAAGCUGUCCCAAUGGGAGCUGC | 245 |
| 826 | 826-844 | GCUGUCCCAAUGGGAGCUGCUGG | 246 |
| 847 | 847-865 | GGGGUGCAGGAGAGGAGAACUGC | 247 |
| 871 | 871-889 | AGAAACUGACCAAAAUCAUCUGU | 248 |
| 872 | 872-890 | GAAACUGACCAAAAUCAUCUGUG | 249 |
| 873 | 873-891 | AAACUGACCAAAAUCAUCUGUGC | 250 |
| 877 | 877-895 | UGACCAAAAUCAUCUGUGCCCAG | 251 |
| 878 | 878-896 | GACCAAAAUCAUCUGUGCCCAGC | 252 |
| 881 | 881-899 | CAAAAUCAUCUGUGCCCAGCAGU | 253 |
| 890 | 890-908 | CUGUGCCCAGCAGUGCUCCGGGC | 254 |
| 892 | 892-910 | GUGCCCAGCAGUGCUCCGGGCGC | 255 |
| 929 | 929-947 | CCCCAGUGACUGCUGCCACAACC | 256 |
| 930 | 930-948 | CCCAGUGACUGCUGCCACAACCA | 257 |
| 979 | 979-997 | GGGAGAGCGACUGCCUGGUCUGC | 258 |
| 980 | 980-998 | GGAGAGCGACUGCCUGGUCUGCC | 259 |
| 981 | 981-999 | GAGAGCGACUGCCUGGUCUGCCG | 260 |
| 982 | 982-1000 | AGAGCGACUGCCUGGUCUGCCGC | 261 |
| 983 | 983-1001 | GAGCGACUGCCUGGUCUGCCGCA | 262 |
| 984 | 984-1002 | AGCGACUGCCUGGUCUGCCGCAA | 263 |
| 989 | 989-1007 | CUGCCUGGUCUGCCGCAAAUUCC | 264 |
| 990 | 990-1008 | UGCCUGGUCUGCCGCAAAUUCCG | 265 |
| 991 | 991-1009 | GCCUGGUCUGCCGCAAAUUCCGA | 266 |
| 992 | 992-1010 | CCUGGUCUGCCGCAAAUUCCGAG | 267 |
| 994 | 994-1012 | UGGUCUGCCGCAAAUUCCGAGAC | 268 |
| 995 | 995-1013 | GGUCUGCCGCAAAUUCCGAGACG | 269 |
| 996 | 996-1014 | GUCUGCCGCAAAUUCCGAGACGA | 270 |
| 997 | 997-1015 | UCUGCCGCAAAUUCCGAGACGAA | 271 |
| 999 | 999-1017 | UGCCGCAAAUUCCGAGACGAAGC | 272 |
| 1004 | 1004-1022 | CAAAUUCCGAGACGAAGCCACGU | 273 |
| 1005 | 1005-1023 | AAAUUCCGAGACGAAGCCACGUG | 274 |
| 1006 | 1006-1024 | AAUUCCGAGACGAAGCCACGUGC | 275 |
| 1007 | 1007-1025 | AUUCCGAGACGAAGCCACGUGCA | 276 |
| 1008 | 1008-1026 | UUCCGAGACGAAGCCACGUGCAA | 277 |
| 1010 | 1010-1028 | CCGAGACGAAGCCACGUGCAAGG | 278 |
| 1013 | 1013-1031 | AGACGAAGCCACGUGCAAGGACA | 279 |
| 1014 | 1014-1032 | GACGAAGCCACGUGCAAGGACAC | 280 |
| 1015 | 1015-1033 | ACGAAGCCACGUGCAAGGACACC | 281 |
| 1016 | 1016-1034 | CGAAGCCACGUGCAAGGACACCU | 282 |
| 1040 | 1040-1058 | CCCCCCACUCAUGCUCUACAACC | 283 |
| 1042 | 1042-1060 | CCCCACUCAUGCUCUACAACCCC | 284 |
| 1044 | 1044-1062 | CCACUCAUGCUCUACAACCCCAC | 285 |
| 1047 | 1047-1065 | CUCAUGCUCUACAACCCCACCAC | 286 |
| 1071 | 1071-1089 | UACCAGAUGGAUGUGAACCCCGA | 287 |
| 1073 | 1073-1091 | CCAGAUGGAUGUGAACCCCGAGG | 288 |
| 1074 | 1074-1092 | CAGAUGGAUGUGAACCCCGAGGG | 289 |
| 1075 | 1075-1093 | AGAUGGAUGUGAACCCCGAGGGC | 290 |
| 1077 | 1077-1095 | AUGGAUGUGAACCCCGAGGGCAA | 291 |
| 1078 | 1078-1096 | UGGAUGUGAACCCCGAGGGCAAA | 292 |
| 1080 | 1080-1098 | GAUGUGAACCCCGAGGGCAAAUA | 293 |
| 1084 | 1084-1102 | UGAACCCCGAGGGCAAAUACAGC | 294 |
| 1085 | 1085-1103 | GAACCCCGAGGGCAAAUACAGCU | 295 |
| 1087 | 1087-1105 | ACCCCGAGGGCAAAUACAGCUUU | 296 |
| 1088 | 1088-1106 | CCCCGAGGGCAAAUACAGCUUUG | 297 |
| 1089 | 1089-1107 | CCCGAGGGCAAAUACAGCUUUGG | 298 |
| 1096 | 1096-1114 | GCAAAUACAGCUUUGGUGCCACC | 299 |
| 1097 | 1097-1115 | CAAAUACAGCUUUGGUGCCACCU | 300 |
| 1098 | 1098-1116 | AAAUACAGCUUUGGUGCCACCUG | 301 |
| 1104 | 1104-1122 | AGCUUUGGUGCCACCUGCGUGAA | 302 |
| 1106 | 1106-1124 | CUUUGGUGCCACCUGCGUGAAGA | 303 |
| 1112 | 1112-1130 | UGCCACCUGCGUGAAGAAGUGUC | 304 |
| 1116 | 1116-1134 | ACCUGCGUGAAGAAGUGUCCCCG | 305 |
| 1117 | 1117-1135 | CCUGCGUGAAGAAGUGUCCCCGU | 306 |
| 1118 | 1118-1136 | CUGCGUGAAGAAGUGUCCCCGUA | 307 |
| 1119 | 1119-1137 | UGCGUGAAGAAGUGUCCCCGUAA | 308 |
| 1120 | 1120-1138 | GCGUGAAGAAGUGUCCCCGUAAU | 309 |
| 1121 | 1121-1139 | CGUGAAGAAGUGUCCCCGUAAUU | 310 |
| 1122 | 1122-1140 | GUGAAGAAGUGUCCCCGUAAUUA | 311 |
| 1123 | 1123-1141 | UGAAGAAGUGUCCCCGUAAUUAU | 312 |
| 1124 | 1124-1142 | GAAGAAGUGUCCCCGUAAUUAUG | 313 |
| 1125 | 1125-1143 | AAGAAGUGUCCCCGUAAUUAUGU | 314 |
| 1126 | 1126-1144 | AGAAGUGUCCCCGUAAUUAUGUG | 315 |
| 1127 | 1127-1145 | GAAGUGUCCCCGUAAUUAUGUGG | 316 |
| 1128 | 1128-1146 | AAGUGUCCCCGUAAUUAUGUGGU | 317 |
| 1129 | 1129-1147 | AGUGUCCCCGUAAUUAUGUGGUG | 318 |
| 1130 | 1130-1148 | GUGUCCCCGUAAUUAUGUGGUGA | 319 |
| 1132 | 1132-1150 | GUCCCCGUAAUUAUGUGGUGACA | 320 |
| 1134 | 1134-1152 | CCCCGUAAUUAUGUGGUGACAGA | 321 |
| 1136 | 1136-1154 | CCGUAAUUAUGUGGUGACAGAUC | 322 |
| 1137 | 1137-1155 | CGUAAUUAUGUGGUGACAGAUCA | 323 |
| 1138 | 1138-1156 | GUAAUUAUGUGGUGACAGAUCAC | 324 |
| 1139 | 1139-1157 | UAAUUAUGUGGUGACAGAUCACG | 325 |
| 1140 | 1140-1158 | AAUUAUGUGGUGACAGAUCACGG | 326 |
| 1142 | 1142-1160 | UUAUGUGGUGACAGAUCACGGCU | 327 |
| 1145 | 1145-1163 | UGUGGUGACAGAUCACGGCUCGU | 328 |
| 1147 | 1147-1165 | UGGUGACAGAUCACGGCUCGUGC | 329 |
| 1148 | 1148-1166 | GGUGACAGAUCACGGCUCGUGCG | 330 |
| 1149 | 1149-1167 | GUGACAGAUCACGGCUCGUGCGU | 331 |
| 1150 | 1150-1168 | UGACAGAUCACGGCUCGUGCGUC | 332 |
| 1151 | 1151-1169 | GACAGAUCACGGCUCGUGCGUCC | 333 |
| 1152 | 1152-1170 | ACAGAUCACGGCUCGUGCGUCCG | 334 |
| 1153 | 1153-1171 | CAGAUCACGGCUCGUGCGUCCGA | 335 |
| 1154 | 1154-1172 | AGAUCACGGCUCGUGCGUCCGAG | 336 |
| 1155 | 1155-1173 | GAUCACGGCUCGUGCGUCCGAGC | 337 |
| 1156 | 1156-1174 | AUCACGGCUCGUGCGUCCGAGCC | 338 |
| 1157 | 1157-1175 | UCACGGCUCGUGCGUCCGAGCCU | 339 |
| 1160 | 1160-1178 | CGGCUCGUGCGUCCGAGCCUGUG | 340 |
| 1200 | 1200-1218 | AUGGAGGAAGACGGCGUCCGCAA | 341 |
| 1201 | 1201-1219 | UGGAGGAAGACGGCGUCCGCAAG | 342 |
| 1203 | 1203-1221 | GAGGAAGACGGCGUCCGCAAGUG | 343 |
| 1204 | 1204-1222 | AGGAAGACGGCGUCCGCAAGUGU | 344 |
| 1205 | 1205-1223 | GGAAGACGGCGUCCGCAAGUGUA | 345 |
| 1207 | 1207-1225 | AAGACGGCGUCCGCAAGUGUAAG | 346 |
| 1208 | 1208-1226 | AGACGGCGUCCGCAAGUGUAAGA | 347 |
| 1211 | 1211-1229 | CGGCGUCCGCAAGUGUAAGAAGU | 348 |
| 1212 | 1212-1230 | GGCGUCCGCAAGUGUAAGAAGUG | 349 |
| 1213 | 1213-1231 | GCGUCCGCAAGUGUAAGAAGUGC | 350 |
| 1214 | 1214-1232 | CGUCCGCAAGUGUAAGAAGUGCG | 351 |
| 1215 | 1215-1233 | GUCCGCAAGUGUAAGAAGUGCGA | 352 |
| 1216 | 1216-1234 | UCCGCAAGUGUAAGAAGUGCGAA | 353 |
| 1217 | 1217-1235 | CCGCAAGUGUAAGAAGUGCGAAG | 354 |
| 1219 | 1219-1237 | GCAAGUGUAAGAAGUGCGAAGGG | 355 |
| 1220 | 1220-1238 | CAAGUGUAAGAAGUGCGAAGGGC | 356 |
| 1221 | 1221-1239 | AAGUGUAAGAAGUGCGAAGGGCC | 357 |
| 1222 | 1222-1240 | AGUGUAAGAAGUGCGAAGGGCCU | 358 |
| 1223 | 1223-1241 | GUGUAAGAAGUGCGAAGGGCCUU | 359 |
| 1224 | 1224-1242 | UGUAAGAAGUGCGAAGGGCCUUG | 360 |
| 1225 | 1225-1243 | GUAAGAAGUGCGAAGGGCCUUGC | 361 |
| 1226 | 1226-1244 | UAAGAAGUGCGAAGGGCCUUGCC | 362 |
| 1229 | 1229-1247 | GAAGUGCGAAGGGCCUUGCCGCA | 363 |
| 1230 | 1230-1248 | AAGUGCGAAGGGCCUUGCCGCAA | 364 |
| 1231 | 1231-1249 | AGUGCGAAGGGCCUUGCCGCAAA | 365 |
| 1232 | 1232-1250 | GUGCGAAGGGCCUUGCCGCAAAG | 366 |
| 1233 | 1233-1251 | UGCGAAGGGCCUUGCCGCAAAGU | 367 |
| 1235 | 1235-1253 | CGAAGGGCCUUGCCGCAAAGUGU | 368 |
| 1236 | 1236-1254 | GAAGGGCCUUGCCGCAAAGUGUG | 369 |
| 1237 | 1237-1255 | AAGGGCCUUGCCGCAAAGUGUGU | 370 |
| 1238 | 1238-1256 | AGGGCCUUGCCGCAAAGUGUGUA | 371 |
| 1239 | 1239-1257 | GGGCCUUGCCGCAAAGUGUGUAA | 372 |
| 1241 | 1241-1259 | GCCUUGCCGCAAAGUGUGUAACG | 373 |
| 1261 | 1261-1279 | ACGGAAUAGGUAUUGGUGAAUUU | 374 |
| 1262 | 1262-1280 | CGGAAUAGGUAUUGGUGAAUUUA | 375 |
| 1263 | 1263-1281 | GGAAUAGGUAUUGGUGAAUUUAA | 376 |
| 1264 | 1264-1282 | GAAUAGGUAUUGGUGAAUUUAAA | 377 |
| 1266 | 1266-1284 | AUAGGUAUUGGUGAAUUUAAAGA | 378 |
| 1267 | 1267-1285 | UAGGUAUUGGUGAAUUUAAAGAC | 379 |
| 1289 | 1289-1307 | CUCACUCUCCAUAAAUGCUACGA | 380 |
| 1313 | 1313-1331 | UAUUAAACACUUCAAAAACUGCA | 381 |
| 1320 | 1320-1338 | CACUUCAAAAACUGCACCUCCAU | 382 |
| 1321 | 1321-1339 | ACUUCAAAAACUGCACCUCCAUC | 383 |
| 1322 | 1322-1340 | CUUCAAAAACUGCACCUCCAUCA | 384 |
| 1323 | 1323-1341 | UUCAAAAACUGCACCUCCAUCAG | 385 |
| 1324 | 1324-1342 | UCAAAAACUGCACCUCCAUCAGU | 386 |
| 1328 | 1328-1346 | AAACUGCACCUCCAUCAGUGGCG | 387 |
| 1332 | 1332-1350 | UGCACCUCCAUCAGUGGCGAUCU | 388 |
| 1333 | 1333-1351 | GCACCUCCAUCAGUGGCGAUCUC | 389 |
| 1335 | 1335-1353 | ACCUCCAUCAGUGGCGAUCUCCA | 390 |
| 1338 | 1338-1356 | UCCAUCAGUGGCGAUCUCCACAU | 391 |
| 1344 | 1344-1362 | AGUGGCGAUCUCCACAUCCUGCC | 392 |
| 1345 | 1345-1363 | GUGGCGAUCUCCACAUCCUGCCG | 393 |
| 1346 | 1346-1364 | UGGCGAUCUCCACAUCCUGCCGG | 394 |
| 1347 | 1347-1365 | GGCGAUCUCCACAUCCUGCCGGU | 395 |
| 1348 | 1348-1366 | GCGAUCUCCACAUCCUGCCGGUG | 396 |
| 1353 | 1353-1371 | CUCCACAUCCUGCCGGUGGCAUU | 397 |
| 1354 | 1354-1372 | UCCACAUCCUGCCGGUGGCAUUU | 398 |
| 1355 | 1355-1373 | CCACAUCCUGCCGGUGGCAUUUA | 399 |
| 1357 | 1357-1375 | ACAUCCUGCCGGUGGCAUUUAGG | 400 |
| 1360 | 1360-1378 | UCCUGCCGGUGGCAUUUAGGGGU | 401 |
| 1361 | 1361-1379 | CCUGCCGGUGGCAUUUAGGGGUG | 402 |
| 1362 | 1362-1380 | CUGCCGGUGGCAUUUAGGGGUGA | 403 |
| 1363 | 1363-1381 | UGCCGGUGGCAUUUAGGGGUGAC | 404 |
| 1366 | 1366-1384 | CGGUGGCAUUUAGGGGUGACUCC | 405 |
| 1369 | 1369-1387 | UGGCAUUUAGGGGUGACUCCUUC | 406 |
| 1370 | 1370-1388 | GGCAUUUAGGGGUGACUCCUUCA | 407 |
| 1371 | 1371-1389 | GCAUUUAGGGGUGACUCCUUCAC | 408 |
| 1372 | 1372-1390 | CAUUUAGGGGUGACUCCUUCACA | 409 |
| 1373 | 1373-1391 | AUUUAGGGGUGACUCCUUCACAC | 410 |
| 1374 | 1374-1392 | UUUAGGGGUGACUCCUUCACACA | 411 |
| 1404 | 1404-1422 | CCUCUGGAUCCACAGGAACUGGA | 412 |
| 1408 | 1408-1426 | UGGAUCCACAGGAACUGGAUAUU | 413 |
| 1409 | 1409-1427 | GGAUCCACAGGAACUGGAUAUUC | 414 |
| 1411 | 1411-1429 | AUCCACAGGAACUGGAUAUUCUG | 415 |
| 1412 | 1412-1430 | UCCACAGGAACUGGAUAUUCUGA | 416 |
| 1419 | 1419-1437 | GAACUGGAUAUUCUGAAAACCGU | 417 |
| 1426 | 1426-1444 | AUAUUCUGAAAACCGUAAAGGAA | 418 |
| 1427 | 1427-1445 | UAUUCUGAAAACCGUAAAGGAAA | 419 |
| 1430 | 1430-1448 | UCUGAAAACCGUAAAGGAAAUCA | 420 |
| 1431 | 1431-1449 | CUGAAAACCGUAAAGGAAAUCAC | 421 |

TABLE 4
EGFR siRNA Sequences

| | Sequence | | | | |
|---|---|---|---|---|---|
| hs Id | position in | sense strand sequence | SEQ ID | antisense strand sequence | SEQ ID |
| # | NM_005228.3 | (5′-3′) | NO: | (5′-3′) | NO: |
| 68 | 68-86 | CGGCCGGAGUCCCGAGCU | 422 | UAGCUCGGGACUCCGGCC | 423 |
| | | ATT | | GTT | |
| 71 | 71-89 | CCGGAGUCCCGAGCUAGC | 424 | GGCUAGCUCGGGACUCCG | 425 |
| | | CTT | | GTT | |
| 72 | 72-90 | CGGAGUCCCGAGCUAGCC | 426 | GGGCUAGCUCGGGACUCC | 427 |
| | | CTT | | GTT | |
| 73 | 73-91 | GGAGUCCCGAGCUAGCCC | 428 | GGGGCUAGCUCGGGACUC | 429 |
| | | CTT | | CTT | |
| 74 | 74-92 | GAGUCCCGAGCUAGCCCC | 430 | CGGGGCUAGCUCGGGACU | 431 |
| | | GTT | | CTT | |
| 75 | 75-93 | AGUCCCGAGCUAGCCCCG | 432 | CCGGGGCUAGCUCGGGAC | 433 |
| | | GTT | | UTT | |
| 76 | 76-94 | GUCCCGAGCUAGCCCCGG | 434 | GCCGGGGCUAGCUCGGGA | 435 |
| | | CTT | | CTT | |
| 78 | 78-96 | CCCGAGCUAGCCCCGGCG | 436 | CCGCCGGGGCUAGCUCGG | 437 |
| | | GTT | | GTT | |
| 114 | 114-132 | GGACGACAGGCCACCUCG | 438 | ACGAGGUGGCCUGUCGUC | 439 |
| | | UTT | | CTT | |
| 115 | 115-133 | GACGACAGGCCACCUCGU | 440 | GACGAGGUGGCCUGUCG | 441 |
| | | CTT | | UCTT | |
| 116 | 116-134 | ACGACAGGCCACCUCGUC | 442 | CGACGAGGUGGCCUGUCG | 443 |
| | | GTT | | UTT | |
| 117 | 117-135 | CGACAGGCCACCUCGUCG | 444 | CCGACGAGGUGGCCUGUC | 445 |
| | | GTT | | GTT | |
| 118 | 118-136 | GACAGGCCACCUCGUCGG | 446 | GCCGACGAGGUGGCCUGU | 447 |
| | | CTT | | CTT | |
| 120 | 120-138 | CAGGCCACCUCGUCGGCG | 448 | ACGCCGACGAGGUGGCCU | 449 |
| | | UTT | | GTT | |
| 121 | 121-139 | AGGCCACCUCGUCGGCGU | 450 | GACGCCGACGAGGUGGCC | 451 |
| | | CTT | | UTT | |
| 122 | 122-140 | GGCCACCUCGUCGGCGUC | 452 | GGACGCCGACGAGGUGGC | 453 |
| | | CTT | | CTT | |
| 123 | 123-141 | GCCACCUCGUCGGCGUCC | 454 | CGGACGCCGACGAGGUGG | 455 |
| | | GTT | | CTT | |
| 124 | 124-142 | CCACCUCGUCGGCGUCCG | 456 | GCGGACGCCGACGAGGUG | 457 |
| | | CTT | | GTT | |
| 125 | 125-143 | CACCUCGUCGGCGUCCGC | 458 | GGCGGACGCCGACGAGGU | 459 |
| | | CTT | | GTT | |
| 126 | 126-144 | ACCUCGUCGGCGUCCGCC | 460 | GGGCGGACGCCGACGAGG | 461 |
| | | CTT | | UTT | |
| 127 | 127-145 | CCUCGUCGGCGUCCGCCC | 462 | CGGGCGGACGCCGACGAG | 463 |
| | | GTT | | GTT | |
| 128 | 128-146 | CUCGUCGGCGUCCGCCCG | 464 | UCGGGCGGACGCCGACGA | 465 |
| | | ATT | | GTT | |
| 129 | 129-147 | UCGUCGGCGUCCGCCCGA | 466 | CUCGGGCGGACGCCGACG | 467 |
| | | GTT | | ATT | |
| 130 | 130-148 | CGUCGGCGUCCGCCCGAG | 468 | ACUCGGGCGGACGCCGAC | 469 |
| | | UTT | | GTT | |
| 131 | 131-149 | GUCGGCGUCCGCCCGAGU | 470 | GACUCGGGCGGACGCCGA | 471 |
| | | CTT | | CTT | |
| 132 | 132-150 | UCGGCGUCCGCCCGAGUC | 472 | GGACUCGGGCGGACGCCG | 473 |
| | | CTT | | ATT | |
| 135 | 135-153 | GCGUCCGCCCGAGUCCCC | 474 | CGGGGACUCGGGCGGACG | 475 |
| | | GTT | | CTT | |
| 136 | 136-154 | CGUCCGCCCGAGUCCCCG | 476 | GCGGGGACUCGGGCGGAC | 477 |
| | | CTT | | GTT | |
| 141 | 141-159 | GCCCGAGUCCCCGCCUCG | 478 | GCGAGGCGGGGACUCGG | 479 |
| | | CTT | | GCTT | |
| 164 | 164-182 | AACGCCACAACCACCGCG | 480 | GCGCGGUGGUUGUGGCG | 481 |
| | | CTT | | UUTT | |
| 165 | 165-183 | ACGCCACAACCACCGCGC | 482 | UGCGCGGUGGUUGUGGC | 483 |
| | | ATT | | GUTT | |
| 166 | 166-184 | CGCCACAACCACCGCGCA | 484 | GUGCGCGGUGGUUGUGG | 485 |
| | | CTT | | CGTT | |
| 168 | 168-186 | CCACAACCACCGCGCACG | 486 | CCGUGCGCGGUGGUUGU | 487 |
| | | GTT | | GGTT | |
| 169 | 169-187 | CACAACCACCGCGCACGG | 488 | GCCGUGCGCGGUGGUUG | 489 |
| | | CTT | | UGTT | |
| 170 | 170-188 | ACAACCACCGCGCACGGC | 490 | GGCCGUGCGCGGUGGUU | 491 |
| | | CTT | | GUTT | |
| 247 | 247-265 | AUGCGACCCUCCGGGACG | 492 | CCGUCCCGGAGGGUCGCA | 493 |
| | | GTT | | UTT | |
| 248 | 248-266 | UGCGACCCUCCGGGACGG | 494 | GCCGUCCCGGAGGGUCGC | 495 |
| | | CTT | | ATT | |
| 249 | 249-267 | GCGACCCUCCGGGACGGC | 496 | GGCCGUCCCGGAGGGUCG | 497 |
| | | CTT | | CTT | |
| 251 | 251-269 | GACCCUCCGGGACGGCCG | 498 | CCGGCCGUCCCGGAGGGU | 499 |
| | | GTT | | CTT | |
| 252 | 252-270 | ACCCUCCGGGACGGCCGG | 500 | CCCGGCCGUCCCGGAGGG | 501 |
| | | GTT | | UTT | |
| 254 | 254-272 | CCUCCGGGACGGCCGGGG | 502 | GCCCCGGCCGUCCCGGAG | 503 |
| | | CTT | | GTT | |
| 329 | 329-347 | AGAAAGUUUGCCAAGGC | 504 | GUGCCUUGGCAAACUUUC | 505 |
| | | ACTT | | UTT | |
| 330 | 330-348 | GAAAGUUUGCCAAGGCAC | 506 | CGUGCCUUGGCAAACUUU | 507 |
| | | GTT | | CTT | |
| 332 | 332-350 | AAGUUUGCCAAGGCACGA | 508 | CUCGUGCCUUGGCAAACU | 509 |
| | | GTT | | UTT | |
| 333 | 333-351 | AGUUUGCCAAGGCACGAG | 510 | ACUCGUGCCUUGGCAAAC | 511 |
| | | UTT | | UTT | |
| 334 | 334-352 | GUUUGCCAAGGCACGAGU | 512 | UACUCGUGCCUUGGCAAA | 513 |
| | | ATT | | CTT | |
| 335 | 335-353 | UUUGCCAAGGCACGAGUA | 514 | UUACUCGUGCCUUGGCAA | 515 |
| | | ATT | | ATT | |
| 336 | 336-354 | UUGCCAAGGCACGAGUAA | 516 | GUUACUCGUGCCUUGGCA | 517 |
| | | CTT | | ATT | |
| 337 | 337-355 | UGCCAAGGCACGAGUAAC | 518 | UGUUACUCGUGCCUUGGC | 519 |
| | | ATT | | ATT | |
| 338 | 338-356 | GCCAAGGCACGAGUAACA | 520 | UUGUUACUCGUGCCUUG | 521 |
| | | ATT | | GCTT | |
| 361 | 361-379 | ACGCAGUUGGGCACUUUU | 522 | CAAAAGUGCCCAACUGCG | 523 |
| | | GTT | | UTT | |
| 362 | 362-380 | CGCAGUUGGGCACUUUUG | 524 | UCAAAAGUGCCCAACUGC | 525 |
| | | ATT | | GTT | |
| 363 | 363-381 | GCAGUUGGGCACUUUUG | 526 | UUCAAAAGUGCCCAACUG | 527 |
| | | AATT | | CTT | |
| 364 | 364-382 | CAGUUGGGCACUUUUGA | 528 | CUUCAAAAGUGCCCAACU | 529 |
| | | AGTT | | GTT | |
| 365 | 365-383 | AGUUGGGCACUUUUGAA | 530 | UCUUCAAAAGUGCCCAAC | 531 |
| | | GATT | | UTT | |
| 366 | 366-384 | GUUGGGCACUUUUGAAG | 532 | AUCUUCAAAAGUGCCCAA | 533 |
| | | AUTT | | CTT | |
| 367 | 367-385 | UUGGGCACUUUUGAAGA | 534 | GAUCUUCAAAAGUGCCCA | 535 |
| | | UCTT | | ATT | |
| 368 | 368-386 | UGGGCACUUUUGAAGAU | 536 | UGAUCUUCAAAAGUGCCC | 537 |
| | | CATT | | ATT | |
| 369 | 369-387 | GGGCACUUUUGAAGAUC | 538 | AUGAUCUUCAAAAGUGC | 539 |
| | | AUTT | | CCTT | |
| 377 | 377-395 | UUGAAGAUCAUUUUCUC | 540 | CUGAGAAAAUGAUCUUC | 541 |
| | | AGTT | | AATT | |
| 379 | 379-397 | GAAGAUCAUUUUCUCAGC | 542 | GGCUGAGAAAAUGAUCU | 543 |
| | | CTT | | UCTT | |
| 380 | 380-398 | AAGAUCAUUUUCUCAGCC | 544 | AGGCUGAGAAAAUGAUC | 545 |
| | | UTT | | UUTT | |
| 385 | 385-403 | CAUUUUCUCAGCCUCCAG | 546 | UCUGGAGGCUGAGAAAA | 547 |
| | | ATT | | UGTT | |
| 394 | 394-412 | AGCCUCCAGAGGAUGUUC | 548 | UGAACAUCCUCUGGAGGC | 549 |
| | | ATT | | UTT | |
| 396 | 396-414 | CCUCCAGAGGAUGUUCAA | 550 | AUUGAACAUCCUCUGGA | 551 |
| | | UTT | | GGTT | |
| 397 | 397-415 | CUCCAGAGGAUGUUCAAU | 552 | UAUUGAACAUCCUCUGG | 553 |
| | | ATT | | AGTT | |
| 401 | 401-419 | AGAGGAUGUUCAAUAAC | 554 | CAGUUAUUGAACAUCCUC | 555 |
| | | UGTT | | UTT | |
| 403 | 403-421 | AGGAUGUUCAAUAACUG | 556 | CACAGUUAUUGAACAUCC | 557 |
| | | UGTT | | UTT | |
| 407 | 407-425 | UGUUCAAUAACUGUGAG | 558 | ACCUCACAGUUAUUGAAC | 559 |
| | | GUTT | | ATT | |
| 409 | 409-427 | UUCAAUAACUGUGAGGU | 560 | CCACCUCACAGUUAUUGA | 561 |
| | | GGTT | | ATT | |
| 410 | 410-428 | UCAAUAACUGUGAGGUG | 562 | ACCACCUCACAGUUAUUG | 563 |
| | | GUTT | | ATT | |
| 411 | 411-429 | CAAUAACUGUGAGGUGG | 564 | GACCACCUCACAGUUAUU | 565 |
| | | UCTT | | GTT | |
| 412 | 412-430 | AAUAACUGUGAGGUGGU | 566 | GGACCACCUCACAGUUAU | 567 |
| | | CCTT | | UTT | |
| 413 | 413-431 | AUAACUGUGAGGUGGUC | 568 | AGGACCACCUCACAGUUA | 569 |
| | | CUTT | | UTT | |
| 414 | 414-432 | UAACUGUGAGGUGGUCC | 570 | AAGGACCACCUCACAGUU | 571 |
| | | UUTT | | ATT | |
| 416 | 416-434 | ACUGUGAGGUGGUCCUU | 572 | CCAAGGACCACCUCACAG | 573 |
| | | GGTT | | UTT | |
| 418 | 418-436 | UGUGAGGUGGUCCUUGG | 574 | UCCCAAGGACCACCUCAC | 575 |
| | | GATT | | ATT | |
| 419 | 419-437 | GUGAGGUGGUCCUUGGG | 576 | UUCCCAAGGACCACCUCA | 577 |
| | | AATT | | CTT | |
| 425 | 425-443 | UGGUCCUUGGGAAUUUG | 578 | UCCAAAUUCCCAAGGACC | 579 |
| | | GATT | | ATT | |
| 431 | 431-449 | UUGGGAAUUUGGAAAUU | 580 | GUAAUUUCCAAAUUCCCA | 581 |
| | | ACTT | | ATT | |
| 432 | 432-450 | UGGGAAUUUGGAAAUUA | 582 | GGUAAUUUCCAAAUUCCC | 583 |
| | | CCTT | | ATT | |
| 433 | 433-451 | GGGAAUUUGGAAAUUAC | 584 | AGGUAAUUUCCAAAUUC | 585 |
| | | CUTT | | CCTT | |
| 434 | 434-452 | GGAAUUUGGAAAUUACC | 586 | UAGGUAAUUUCCAAAUU | 587 |
| | | UATT | | CCTT | |
| 458 | 458-476 | AGAGGAAUUAUGAUCUU | 588 | GAAAGAUCAUAAUUCCU | 589 |
| | | UCTT | | CUTT | |
| 459 | 459-477 | GAGGAAUUAUGAUCUUU | 590 | GGAAAGAUCAUAAUUCC | 591 |
| | | CCTT | | UCTT | |
| 463 | 463-481 | AAUUAUGAUCUUUCCUUC | 592 | AGAAGGAAAGAUCAUAA | 593 |
| | | UTT | | UUTT | |
| 464 | 464-482 | AUUAUGAUCUUUCCUUCU | 594 | AAGAAGGAAAGAUCAUA | 595 |
| | | UTT | | AUTT | |
| 466 | 466-484 | UAUGAUCUUUCCUUCUUA | 596 | UUAAGAAGGAAAGAUCA | 597 |
| | | ATT | | UATT | |
| 468 | 468-486 | UGAUCUUUCCUUCUUAAA | 598 | CUUUAAGAAGGAAAGAU | 599 |
| | | GTT | | CATT | |
| 471 | 471-489 | UCUUUCCUUCUUAAAGAC | 600 | GGUCUUUAAGAAGGAAA | 601 |
| | | CTT | | GATT | |
| 476 | 476-494 | CCUUCUUAAAGACCAUCC | 602 | UGGAUGGUCUUUAAGAA | 603 |
| | | ATT | | GGTT | |
| 477 | 477-495 | CUUCUUAAAGACCAUCCA | 604 | CUGGAUGGUCUUUAAGA | 605 |
| | | GTT | | AGTT | |
| 479 | 479-497 | UCUUAAAGACCAUCCAGG | 606 | UCCUGGAUGGUCUUUAA | 607 |
| | | ATT | | GATT | |
| 481 | 481-499 | UUAAAGACCAUCCAGGAG | 608 | CCUCCUGGAUGGUCUUUA | 609 |
| | | GTT | | ATT | |
| 482 | 482-500 | UAAAGACCAUCCAGGAGG | 610 | ACCUCCUGGAUGGUCUUU | 611 |
| | | UTT | | ATT | |
| 492 | 492-510 | CCAGGAGGUGGCUGGUU | 612 | AUAACCAGCCACCUCCUG | 613 |
| | | AUTT | | GTT | |
| 493 | 493-511 | CAGGAGGUGGCUGGUUA | 614 | CAUAACCAGCCACCUCCU | 615 |
| | | UGTT | | GTT | |
| 494 | 494-512 | AGGAGGUGGCUGGUUAU | 616 | ACAUAACCAGCCACCUCC | 617 |
| | | GUTT | | UTT | |
| 495 | 495-513 | GGAGGUGGCUGGUUAUG | 618 | GACAUAACCAGCCACCUC | 619 |
| | | UCTT | | CTT | |
| 496 | 496-514 | GAGGUGGCUGGUUAUGU | 620 | GGACAUAACCAGCCACCU | 621 |
| | | CCTT | | CTT | |
| 497 | 497-515 | AGGUGGCUGGUUAUGUC | 622 | AGGACAUAACCAGCCACC | 623 |
| | | CUTT | | UTT | |
| 499 | 499-517 | GUGGCUGGUUAUGUCCUC | 624 | UGAGGACAUAACCAGCCA | 625 |
| | | ATT | | CTT | |
| 520 | 520-538 | GCCCUCAACACAGUGGAG | 626 | GCUCCACUGUGUUGAGG | 627 |
| | | CTT | | GCTT | |
| 542 | 542-560 | UUCCUUUGGAAAACCUGC | 628 | UGCAGGUUUUCCAAAGG | 629 |
| | | ATT | | AATT | |
| 543 | 543-561 | UCCUUUGGAAAACCUGCA | 630 | CUGCAGGUUUUCCAAAG | 631 |
| | | GTT | | GATT | |
| 550 | 550-568 | GAAAACCUGCAGAUCAUC | 632 | UGAUGAUCUGCAGGUUU | 633 |
| | | ATT | | UCTT | |
| 551 | 551-569 | AAAACCUGCAGAUCAUCA | 634 | CUGAUGAUCUGCAGGUU | 635 |
| | | GTT | | UUTT | |
| 553 | 553-571 | AACCUGCAGAUCAUCAGA | 636 | CUCUGAUGAUCUGCAGG | 637 |
| | | GTT | | UUTT | |
| 556 | 556-574 | CUGCAGAUCAUCAGAGGA | 638 | UUCCUCUGAUGAUCUGCA | 639 |
| | | ATT | | GTT | |
| 586 | 586-604 | GAAAAUUCCUAUGCCUUA | 640 | CUAAGGCAUAGGAAUUU | 641 |
| | | GTT | | UCTT | |
| 587 | 587-605 | AAAAUUCCUAUGCCUUAG | 642 | GCUAAGGCAUAGGAAUU | 643 |
| | | CTT | | UUTT | |
| 589 | 589-607 | AAUUCCUAUGCCUUAGCA | 644 | CUGCUAAGGCAUAGGAA | 645 |
| | | GTT | | UUTT | |
| 592 | 592-610 | UCCUAUGCCUUAGCAGUC | 646 | AGACUGCUAAGGCAUAG | 647 |
| | | UTT | | GATT | |
| 593 | 593-611 | CCUAUGCCUUAGCAGUCU | 648 | AAGACUGCUAAGGCAUA | 649 |
| | | UTT | | GGTT | |
| 594 | 594-612 | CUAUGCCUUAGCAGUCUU | 650 | UAAGACUGCUAAGGCAU | 651 |
| | | ATT | | AGTT | |
| 596 | 596-614 | AUGCCUUAGCAGUCUUAU | 652 | GAUAAGACUGCUAAGGC | 653 |
| | | CTT | | AUTT | |
| 597 | 597-615 | UGCCUUAGCAGUCUUAUC | 654 | AGAUAAGACUGCUAAGG | 655 |
| | | UTT | | CATT | |
| 598 | 598-616 | GCCUUAGCAGUCUUAUCU | 656 | UAGAUAAGACUGCUAAG | 657 |
| | | ATT | | GCTT | |
| 599 | 599-617 | CCUUAGCAGUCUUAUCUA | 658 | UUAGAUAAGACUGCUAA | 659 |
| | | ATT | | GGTT | |
| 600 | 600-618 | CUUAGCAGUCUUAUCUAA | 660 | GUUAGAUAAGACUGCUA | 661 |
| | | CTT | | AGTT | |
| 601 | 601-619 | UUAGCAGUCUUAUCUAAC | 662 | AGUUAGAUAAGACUGCU | 663 |
| | | UTT | | AATT | |
| 602 | 602-620 | UAGCAGUCUUAUCUAACU | 664 | UAGUUAGAUAAGACUGC | 665 |
| | | ATT | | UATT | |
| 603 | 603-621 | AGCAGUCUUAUCUAACUA | 666 | AUAGUUAGAUAAGACUG | 667 |
| | | UTT | | CUTT | |
| 604 | 604-622 | GCAGUCUUAUCUAACUAU | 668 | CAUAGUUAGAUAAGACU | 669 |
| | | GTT | | GCTT | |
| 605 | 605-623 | CAGUCUUAUCUAACUAUG | 670 | UCAUAGUUAGAUAAGAC | 671 |
| | | ATT | | UGTT | |
| 608 | 608-626 | UCUUAUCUAACUAUGAU | 672 | GCAUCAUAGUUAGAUAA | 673 |
| | | GCTT | | GATT | |
| 609 | 609-627 | CUUAUCUAACUAUGAUGC | 674 | UGCAUCAUAGUUAGAUA | 675 |
| | | ATT | | AGTT | |
| 610 | 610-628 | UUAUCUAACUAUGAUGC | 676 | UUGCAUCAUAGUUAGAU | 677 |
| | | AATT | | AATT | |
| 611 | 611-629 | UAUCUAACUAUGAUGCA | 678 | UUUGCAUCAUAGUUAGA | 679 |
| | | AATT | | UATT | |
| 612 | 612-630 | AUCUAACUAUGAUGCAA | 680 | AUUUGCAUCAUAGUUAG | 681 |
| | | AUTT | | AUTT | |
| 613 | 613-631 | UCUAACUAUGAUGCAAA | 682 | UAUUUGCAUCAUAGUUA | 683 |
| | | UATT | | GATT | |
| 614 | 614-632 | CUAACUAUGAUGCAAAU | 684 | UUAUUUGCAUCAUAGUU | 685 |
| | | AATT | | AGTT | |
| 616 | 616-634 | AACUAUGAUGCAAAUAA | 686 | UUUUAUUUGCAUCAUAG | 687 |
| | | AATT | | UUTT | |
| 622 | 622-640 | GAUGCAAAUAAAACCGG | 688 | GUCCGGUUUUAUUUGCA | 689 |
| | | ACTT | | UCTT | |
| 623 | 623-641 | AUGCAAAUAAAACCGGAC | 690 | AGUCCGGUUUUAUUUGC | 691 |
| | | UTT | | AUTT | |
| 624 | 624-642 | UGCAAAUAAAACCGGACU | 692 | CAGUCCGGUUUUAUUUG | 693 |
| | | GTT | | CATT | |
| 626 | 626-644 | CAAAUAAAACCGGACUGA | 694 | UUCAGUCCGGUUUUAUU | 695 |
| | | ATT | | UGTT | |
| 627 | 627-645 | AAAUAAAACCGGACUGA | 696 | CUUCAGUCCGGUUUUAU | 697 |
| | | AGTT | | UUTT | |
| 628 | 628-646 | AAUAAAACCGGACUGAA | 698 | CCUUCAGUCCGGUUUUAU | 699 |
| | | GGTT | | UTT | |
| 630 | 630-648 | UAAAACCGGACUGAAGG | 700 | CUCCUUCAGUCCGGUUUU | 701 |
| | | AGTT | | ATT | |
| 631 | 631-649 | AAAACCGGACUGAAGGA | 702 | GCUCCUUCAGUCCGGUUU | 703 |
| | | GCTT | | UTT | |
| 632 | 632-650 | AAACCGGACUGAAGGAGC | 704 | AGCUCCUUCAGUCCGGUU | 705 |
| | | UTT | | UTT | |
| 633 | 633-651 | AACCGGACUGAAGGAGCU | 706 | CAGCUCCUUCAGUCCGGU | 707 |
| | | GTT | | UTT | |
| 644 | 644-662 | AGGAGCUGCCCAUGAGAA | 708 | UUUCUCAUGGGCAGCUCC | 709 |
| | | ATT | | UTT | |
| 665 | 665-683 | UACAGGAAAUCCUGCAUG | 710 | CCAUGCAGGAUUUCCUGU | 711 |
| | | GTT | | ATT | |
| 668 | 668-686 | AGGAAAUCCUGCAUGGCG | 712 | GCGCCAUGCAGGAUUUCC | 713 |
| | | CTT | | UTT | |
| 669 | 669-687 | GGAAAUCCUGCAUGGCGC | 714 | GGCGCCAUGCAGGAUUUC | 715 |
| | | CTT | | CTT | |
| 670 | 670-688 | GAAAUCCUGCAUGGCGCC | 716 | CGGCGCCAUGCAGGAUUU | 717 |
| | | GTT | | CTT | |
| 671 | 671-689 | AAAUCCUGCAUGGCGCCG | 718 | ACGGCGCCAUGCAGGAUU | 719 |
| | | UTT | | UTT | |
| 672 | 672 690 | AAUCCUGCAUGGCGCCGU | 720 | CACGGCGCCAUGCAGGAU | 721 |
| | | GTT | | UTT | |
| 674 | 674-692 | UCCUGCAUGGCGCCGUGC | 722 | CGCACGGCGCCAUGCAGG | 723 |
| | | GTT | | ATT | |
| 676 | 676-694 | CUGCAUGGCGCCGUGCGG | 724 | ACCGCACGGCGCCAUGCA | 725 |
| | | UTT | | GTT | |
| 677 | 677-695 | UGCAUGGCGCCGUGCGGU | 726 | AACCGCACGGCGCCAUGC | 727 |
| | | UTT | | ATT | |
| 678 | 678-696 | GCAUGGCGCCGUGCGGUU | 728 | GAACCGCACGGCGCCAUG | 729 |
| | | CTT | | CTT | |
| 680 | 680-698 | AUGGCGCCGUGCGGUUCA | 730 | CUGAACCGCACGGCGCCA | 731 |
| | | GTT | | UTT | |
| 681 | 681-699 | UGGCGCCGUGCGGUUCAG | 732 | GCUGAACCGCACGGCGCC | 733 |
| | | CTT | | ATT | |
| 682 | 682-700 | GGCGCCGUGCGGUUCAGC | 734 | UGCUGAACCGCACGGCGC | 735 |
| | | ATT | | CTT | |
| 683 | 683-701 | GCGCCGUGCGGUUCAGCA | 736 | UUGCUGAACCGCACGGCG | 737 |
| | | ATT | | CTT | |
| 684 | 684-702 | CGCCGUGCGGUUCAGCAA | 738 | GUUGCUGAACCGCACGGC | 739 |
| | | CTT | | GTT | |
| 685 | 685-703 | GCCGUGCGGUUCAGCAAC | 740 | UGUUGCUGAACCGCACGG | 741 |
| | | ATT | | CTT | |
| 686 | 686-704 | CCGUGCGGUUCAGCAACA | 742 | UUGUUGCUGAACCGCACG | 743 |
| | | ATT | | GTT | |
| 688 | 688-706 | GUGCGGUUCAGCAACAAC | 744 | GGUUGUUGCUGAACCGC | 745 |
| | | CTT | | ACTT | |
| 690 | 690-708 | GCGGUUCAGCAACAACCC | 746 | AGGGUUGUUGCUGAACC | 747 |
| | | UTT | | GCTT | |
| 692 | 692-710 | GGUUCAGCAACAACCCUG | 748 | GCAGGGUUGUUGCUGAA | 749 |
| | | CTT | | CCTT | |
| 698 | 698-716 | GCAACAACCCUGCCCUGU | 750 | CACAGGGCAGGGUUGUU | 751 |
| | | GTT | | GCTT | |
| 700 | 700-718 | AACAACCCUGCCCUGUGC | 752 | UGCACAGGGCAGGGUUG | 753 |
| | | ATT | | UUTT | |
| 719 | 719-737 | ACGUGGAGAGCAUCCAGU | 754 | CACUGGAUGCUCUCCACG | 755 |
| | | GTT | | UTT | |
| 720 | 720-738 | CGUGGAGAGCAUCCAGUG | 756 | CCACUGGAUGCUCUCCAC | 757 |
| | | GTT | | GTT | |
| 721 | 721-739 | GUGGAGAGCAUCCAGUG | 758 | GCCACUGGAUGCUCUCCA | 759 |
| | | CTT | CTT | | |
| 724 | 724-742 | GAGAGCAUCCAGUGGCGG | 760 | CCCGCCACUGGAUGCUCU | 761 |
| | | GTT | | CTT | |
| 725 | 725-743 | AGAGCAUCCAGUGGCGGG | 762 | UCCCGCCACUGGAUGCUC | 763 |
| | | ATT | | UTT | |
| 726 | 726-744 | GAGCAUCCAGUGGCGGGA | 764 | GUCCCGCCACUGGAUGCU | 765 |
| | | CTT | | CTT | |
| 733 | 733-751 | CAGUGGCGGGACAUAGUC | 766 | UGACUAUGUCCCGCCACU | 767 |
| | | ATT | | GTT | |
| 734 | 734-752 | AGUGGCGGGACAUAGUC | 768 | CUGACUAUGUCCCGCCAC | 769 |
| | | AGTT | | UTT | |
| 736 | 736-754 | UGGCGGGACAUAGUCAGC | 770 | UGCUGACUAUGUCCCGCC | 771 |
| | | ATT | | ATT | |
| 737 | 737-755 | GGCGGGACAUAGUCAGCA | 772 | CUGCUGACUAUGUCCCGC | 773 |
| | | GTT | | CTT | |
| 763 | 763-781 | CUCAGCAACAUGUCGAUG | 774 | CCAUCGACAUGUUGCUGA | 775 |
| | | GTT | | GTT | |
| 765 | 765-783 | CAGCAACAUGUCGAUGGA | 776 | GUCCAUCGACAUGUUGCU | 777 |
| | | CTT | | GTT | |
| 766 | 766-784 | AGCAACAUGUCGAUGGAC | 778 | AGUCCAUCGACAUGUUGC | 779 |
| | | UTT | | UTT | |
| 767 | 767-785 | GCAACAUGUCGAUGGACU | 780 | AAGUCCAUCGACAUGUU | 781 |
| | | UTT | | GCTT | |
| 769 | 769-787 | AACAUGUCGAUGGACUUC | 782 | GGAAGUCCAUCGACAUG | 783 |
| | | CTT | | UUTT | |
| 770 | 770-788 | ACAUGUCGAUGGACUUCC | 784 | UGGAAGUCCAUCGACAU | 785 |
| | | ATT | | GUTT | |
| 771 | 771-789 | CAUGUCGAUGGACUUCCA | 786 | CUGGAAGUCCAUCGACAU | 787 |
| | | GTT | | GTT | |
| 772 | 772-790 | AUGUCGAUGGACUUCCAG | 788 | UCUGGAAGUCCAUCGACA | 789 |
| | | ATT | | UTT | |
| 775 | 775-793 | UCGAUGGACUUCCAGAAC | 790 | GGUUCUGGAAGUCCAUC | 791 |
| | | CTT | | GATT | |
| 789 | 789-807 | GAACCACCUGGGCAGCUG | 792 | GCAGCUGCCCAGGUGGUU | 793 |
| | | CTT | | CTT | |
| 798 | 798-816 | GGGCAGCUGCCAAAAGUG | 794 | ACACUUUUGGCAGCUGCC | 795 |
| | | UTT | | CTT | |
| 800 | 800-818 | GCAGCUGCCAAAAGUGUG | 796 | UCACACUUUUGGCAGCUG | 797 |
| | | ATT | | CTT | |
| 805 | 805-823 | UGCCAAAAGUGUGAUCCA | 798 | UUGGAUCACACUUUUGG | 799 |
| | | ATT | | CATT | |
| 806 | 806-824 | GCCAAAAGUGUGAUCCAA | 800 | CUUGGAUCACACUUUUG | 801 |
| | | GTT | | GCTT | |
| 807 | 807-825 | CCAAAAGUGUGAUCCAAG | 802 | GCUUGGAUCACACUUUU | 803 |
| | | CTT | | GGTT | |
| 810 | 810-828 | AAAGUGUGAUCCAAGCU | 804 | ACAGCUUGGAUCACACUU | 805 |
| | | GUTT | | UTT | |
| 814 | 814-832 | UGUGAUCCAAGCUGUCCC | 806 | UGGGACAGCUUGGAUCA | 807 |
| | | ATT | | CATT | |
| 815 | 815-833 | GUGAUCCAAGCUGUCCCA | 808 | UUGGGACAGCUUGGAUC | 809 |
| | | ATT | | ACTT | |
| 817 | 817-835 | GAUCCAAGCUGUCCCAAU | 810 | CAUUGGGACAGCUUGGA | 811 |
| | | GTT | | UCTT | |
| 818 | 818-836 | AUCCAAGCUGUCCCAAUG | 812 | CCAUUGGGACAGCUUGG | 813 |
| | | GTT | | AUTT | |
| 819 | 819-837 | UCCAAGCUGUCCCAAUGG | 814 | CCCAUUGGGACAGCUUGG | 815 |
| | | GTT | | ATT | |
| 820 | 820-838 | CCAAGCUGUCCCAAUGGG | 816 | UCCCAUUGGGACAGCUUG | 817 |
| | | ATT | | GTT | |
| 821 | 821-839 | CAAGCUGUCCCAAUGGGA | 818 | CUCCCAUUGGGACAGCUU | 819 |
| | | GTT | | GTT | |
| 823 | 823-841 | AGCUGUCCCAAUGGGAGC | 820 | AGCUCCCAUUGGGACAGC | 821 |
| | | UTT | | UTT | |
| 826 | 826-844 | UGUCCCAAUGGGAGCUGC | 822 | AGCAGCUCCCAUUGGGAC | 823 |
| | | UTT | | ATT | |
| 847 | 847-865 | GGUGCAGGAGAGGAGAA | 824 | AGUUCUCCUCUCCUGCAC | 825 |
| | | CUTT | | CTT | |
| 871 | 871-889 | AAACUGACCAAAAUCAUC | 826 | AGAUGAUUUUGGUCAGU | 827 |
| | | UTT | | UUTT | |
| 872 | 872-890 | AACUGACCAAAAUCAUCU | 828 | CAGAUGAUUUUGGUCAG | 829 |
| | | GTT | | UUTT | |
| 873 | 873-891 | ACUGACCAAAAUCAUCUG | 830 | ACAGAUGAUUUUGGUCA | 831 |
| | | UTT | | GUTT | |
| 877 | 877-895 | ACCAAAAUCAUCUGUGCC | 832 | GGGCACAGAUGAUUUUG | 833 |
| | | CTT | | GUTT | |
| 878 | 878-896 | CCAAAAUCAUCUGUGCCC | 834 | UGGGCACAGAUGAUUUU | 835 |
| | | ATT | | GGTT | |
| 881 | 881-899 | AAAUCAUCUGUGCCCAGC | 836 | UGCUGGGCACAGAUGAU | 837 |
| | | ATT | | UUTT | |
| 890 | 890-908 | GUGCCCAGCAGUGCUCCG | 838 | CCGGAGCACUGCUGGGCA | 839 |
| | | GTT | | CTT | |
| 892 | 892-910 | GCCCAGCAGUGCUCCGGG | 840 | GCCCGGAGCACUGCUGGG | 841 |
| | | CTT | | CTT | |
| 929 | 929-947 | CCAGUGACUGCUGCCACA | 842 | UUGUGGCAGCAGUCACU | 843 |
| | | ATT | | GGTT | |
| 930 | 930-948 | CAGUGACUGCUGCCACAA | 844 | GUUGUGGCAGCAGUCAC | 845 |
| | | CTT | | UGTT | |
| 979 | 979-997 | GAGAGCGACUGCCUGGUC | 846 | AGACCAGGCAGUCGCUCU | 847 |
| | | UTT | | CTT | |
| 980 | 980-998 | AGAGCGACUGCCUGGUCU | 848 | CAGACCAGGCAGUCGCUC | 849 |
| | | GTT | | UTT | |
| 981 | 981-999 | GAGCGACUGCCUGGUCUG | 850 | GCAGACCAGGCAGUCGCU | 851 |
| | | CTT | | CTT | |
| 982 | 982-1000 | AGCGACUGCCUGGUCUGC | 852 | GGCAGACCAGGCAGUCGC | 853 |
| | | CTT | | UTT | |
| 983 | 983-1001 | GCGACUGCCUGGUCUGCC | 854 | CGGCAGACCAGGCAGUCG | 855 |
| | | GTT | | CTT | |
| 984 | 984-1002 | CGACUGCCUGGUCUGCCG | 856 | GCGGCAGACCAGGCAGUC | 857 |
| | | CTT | | GTT | |
| 989 | 989-1007 | GCCUGGUCUGCCGCAAAU | 858 | AAUUUGCGGCAGACCAG | 859 |
| | | UTT | | GCTT | |
| 990 | 990-1008 | CCUGGUCUGCCGCAAAUU | 860 | GAAUUUGCGGCAGACCA | 861 |
| | | CTT | | GGTT | |
| 991 | 991-1009 | CUGGUCUGCCGCAAAUUC | 862 | GGAAUUUGCGGCAGACC | 863 |
| | | CTT | | AGTT | |
| 992 | 992-1010 | UGGUCUGCCGCAAAUUCC | 864 | CGGAAUUUGCGGCAGACC | 865 |
| | | GTT | | ATT | |
| 994 | 994-1012 | GUCUGCCGCAAAUUCCGA | 866 | CUCGGAAUUUGCGGCAG | 867 |
| | | GTT | | ACTT | |
| 995 | 995-1013 | UCUGCCGCAAAUUCCGAG | 868 | UCUCGGAAUUUGCGGCA | 869 |
| | | ATT | | GATT | |
| 996 | 996-1014 | CUGCCGCAAAUUCCGAGA | 870 | GUCUCGGAAUUUGCGGC | 871 |
| | | CTT | | AGTT | |
| 997 | 997-1015 | UGCCGCAAAUUCCGAGAC | 872 | CGUCUCGGAAUUUGCGGC | 873 |
| | | GTT | | ATT | |
| 999 | 999-1017 | CCGCAAAUUCCGAGACGA | 874 | UUCGUCUCGGAAUUUGC | 875 |
| | | ATT | | GGTT | |
| 1004 | 1004-1022 | AAUUCCGAGACGAAGCCA | 876 | GUGGCUUCGUCUCGGAA | 877 |
| | | CTT | | UUTT | |
| 1005 | 1005-1023 | AUUCCGAGACGAAGCCAC | 878 | CGUGGCUUCGUCUCGGAA | 879 |
| | | GTT | | UTT | |
| 1006 | 1006-1024 | UUCCGAGACGAAGCCACG | 880 | ACGUGGCUUCGUCUCGGA | 881 |
| | | UTT | | ATT | |
| 1007 | 1007-1025 | UCCGAGACGAAGCCACGU | 882 | CACGUGGCUUCGUCUCGG | 883 |
| | | GTT | | ATT | |
| 1008 | 1008-1026 | CCGAGACGAAGCCACGUG | 884 | GCACGUGGCUUCGUCUCG | 885 |
| | | CTT | | GTT | |
| 1010 | 1010-1028 | GAGACGAAGCCACGUGCA | 886 | UUGCACGUGGCUUCGUCU | 887 |
| | | ATT | | CTT | |
| 1013 | 1013-1031 | ACGAAGCCACGUGCAAGG | 888 | UCCUUGCACGUGGCUUCG | 889 |
| | | ATT | | UTT | |
| 1014 | 1014-1032 | CGAAGCCACGUGCAAGGA | 890 | GUCCUUGCACGUGGCUUC | 891 |
| | | CTT | | GTT | |
| 1015 | 1015-1033 | GAAGCCACGUGCAAGGAC | 892 | UGUCCUUGCACGUGGCUU | 893 |
| | | ATT | | CTT | |
| 1016 | 1016-1034 | AAGCCACGUGCAAGGACA | 894 | GUGUCCUUGCACGUGGCU | 895 |
| | | CTT | | UTT | |
| 1040 | 1040-1058 | CCCCACUCAUGCUCUACA | 896 | UUGUAGAGCAUGAGUGG | 897 |
| | | ATT | | GGTT | |
| 1042 | 1042-1060 | CCACUCAUGCUCUACAAC | 898 | GGUUGUAGAGCAUGAGU | 899 |
| | | CTT | | GGTT | |
| 1044 | 1044-1062 | ACUCAUGCUCUACAACCC | 900 | GGGGUUGUAGAGCAUGA | 901 |
| | | CTT | | GUTT | |
| 1047 | 1047-1065 | CAUGCUCUACAACCCCAC | 902 | GGUGGGGUUGUAGAGCA | 903 |
| | | CTT | | UGTT | |
| 1071 | 1071-1089 | CCAGAUGGAUGUGAACCC | 904 | GGGGUUCACAUCCAUCUG | 905 |
| | | CTT | | GTT | |
| 1073 | 1073-1091 | AGAUGGAUGUGAACCCCG | 906 | UCGGGGUUCACAUCCAUC | 907 |
| | | ATT | | UTT | |
| 1074 | 1074-1092 | GAUGGAUGUGAACCCCGA | 908 | CUCGGGGUUCACAUCCAU | 909 |
| | | GTT | | CTT | |
| 1075 | 1075-1093 | AUGGAUGUGAACCCCGAG | 910 | CCUCGGGGUUCACAUCCA | 911 |
| | | GTT | | UTT | |
| 1077 | 1077-1095 | GGAUGUGAACCCCGAGGG | 912 | GCCCUCGGGGUUCACAUC | 913 |
| | | CTT | | CTT | |
| 1078 | 1078-1096 | GAUGUGAACCCCGAGGGC | 914 | UGCCCUCGGGGUUCACAU | 915 |
| | | ATT | | CTT | |
| 1080 | 1080-1098 | UGUGAACCCCGAGGGCAA | 916 | UUUGCCCUCGGGGUUCAC | 917 |
| | | ATT | | ATT | |
| 1084 | 1084-1102 | AACCCCGAGGGCAAAUAC | 918 | UGUAUUUGCCCUCGGGG | 919 |
| | | ATT | | UUTT | |
| 1085 | 1085-1103 | ACCCCGAGGGCAAAUACA | 920 | CUGUAUUUGCCCUCGGGG | 921 |
| | | GTT | | UTT | |
| 1087 | 1087-1105 | CCCGAGGGCAAAUACAGC | 922 | AGCUGUAUUUGCCCUCGG | 923 |
| | | UTT | | GTT | |
| 1088 | 1088-1106 | CCGAGGGCAAAUACAGCU | 924 | AAGCUGUAUUUGCCCUCG | 925 |
| | | UTT | | GTT | |
| 1089 | 1089-1107 | CGAGGGCAAAUACAGCUU | 926 | AAAGCUGUAUUUGCCCUC | 927 |
| | | UTT | | GTT | |
| 1096 | 1096-1114 | AAAUACAGCUUUGGUGCC | 928 | UGGCACCAAAGCUGUAU | 929 |
| | | ATT | | UUTT | |
| 1097 | 1097-1115 | AAUACAGCUUUGGUGCCA | 930 | GUGGCACCAAAGCUGUA | 931 |
| | | CTT | | UUTT | |
| 1098 | 1098-1116 | AUACAGCUUUGGUGCCAC | 932 | GGUGGCACCAAAGCUGU | 933 |
| | | CTT | | AUTT | |
| 1104 | 1104-1122 | CUUUGGUGCCACCUGCGU | 934 | CACGCAGGUGGCACCAAA | 935 |
| | | GTT | | GTT | |
| 1106 | 1106-1124 | UUGGUGCCACCUGCGUGA | 936 | UUCACGCAGGUGGCACCA | 937 |
| | | ATT | | ATT | |
| 1112 | 1112-1130 | CCACCUGCGUGAAGAAGU | 938 | CACUUCUUCACGCAGGUG | 939 |
| | | GTT | | GTT | |
| 1116 | 1116-1134 | CUGCGUGAAGAAGUGUCC | 940 | GGGACACUUCUUCACGCA | 941 |
| | | CTT | | GTT | |
| 1117 | 1117-1135 | UGCGUGAAGAAGUGUCCC | 942 | GGGGACACUUCUUCACGC | 943 |
| | | CTT | | ATT | |
| 1118 | 1118-1136 | GCGUGAAGAAGUGUCCCC | 944 | CGGGGACACUUCUUCACG | 945 |
| | | GTT | | CTT | |
| 1119 | 1119-1137 | CGUGAAGAAGUGUCCCCG | 946 | ACGGGGACACUUCUUCAC | 947 |
| | | UTT | | GTT | |
| 1120 | 1120-1138 | GUGAAGAAGUGUCCCCGU | 948 | UACGGGGACACUUCUUCA | 949 |
| | | ATT | | CTT | |
| 1121 | 1121-1139 | UGAAGAAGUGUCCCCGUA | 950 | UUACGGGGACACUUCUUC | 951 |
| | | ATT | | ATT | |
| 1122 | 1122-1140 | GAAGAAGUGUCCCCGUAA | 952 | AUUACGGGGACACUUCU | 953 |
| | | UTT | | UCTT | |
| 1123 | 1123-1141 | AAGAAGUGUCCCCGUAAU | 954 | AAUUACGGGGACACUUC | 955 |
| | | UTT | | UUTT | |
| 1124 | 1124-1142 | AGAAGUGUCCCCGUAAUU | 956 | UAAUUACGGGGACACUU | 957 |
| | | ATT | | CUTT | |
| 1125 | 1125-1143 | GAAGUGUCCCCGUAAUUA | 958 | AUAAUUACGGGGACACU | 959 |
| | | UTT | | UCTT | |
| 1126 | 1126-1144 | AAGUGUCCCCGUAAUUAU | 960 | CAUAAUUACGGGGACAC | 961 |
| | | GTT | | UUTT | |
| 1127 | 1127-1145 | AGUGUCCCCGUAAUUAUG | 962 | ACAUAAUUACGGGGACA | 963 |
| | | UTT | | CUTT | |
| 1128 | 1128-1146 | GUGUCCCCGUAAUUAUGU | 964 | CACAUAAUUACGGGGAC | 965 |
| | | GTT | | ACTT | |
| 1129 | 1129-1147 | UGUCCCCGUAAUUAUGUG | 966 | CCACAUAAUUACGGGGAC | 967 |
| | | GTT | | ATT | |
| 1130 | 1130-1148 | GUCCCCGUAAUUAUGUGG | 968 | ACCACAUAAUUACGGGG | 969 |
| | | UTT | | ACTT | |
| 1132 | 1132-1150 | CCCCGUAAUUAUGUGGUG | 970 | UCACCACAUAAUUACGGG | 971 |
| | | ATT | | GTT | |
| 1134 | 1134-1152 | CCGUAAUUAUGUGGUGA | 972 | UGUCACCACAUAAUUACG | 973 |
| | | CATT | | GTT | |
| 1136 | 1136-1154 | GUAAUUAUGUGGUGACA | 974 | UCUGUCACCACAUAAUUA | 975 |
| | | GATT | | CTT | |
| 1137 | 1137-1155 | UAAUUAUGUGGUGACAG | 976 | AUCUGUCACCACAUAAUU | 977 |
| | | AUTT | | ATT | |
| 1138 | 1138-1156 | AAUUAUGUGGUGACAGA | 978 | GAUCUGUCACCACAUAAU | 979 |
| | | UCTT | | UTT | |
| 1139 | 1139-1157 | AUUAUGUGGUGACAGAU | 980 | UGAUCUGUCACCACAUAA | 981 |
| | | CATT | | UTT | |
| 1140 | 1140-1158 | UUAUGUGGUGACAGAUC | 982 | GUGAUCUGUCACCACAUA | 983 |
| | | ACTT | | ATT | |
| 1142 | 1142-1160 | AUGUGGUGACAGAUCAC | 984 | CCGUGAUCUGUCACCACA | 985 |
| | | GGTT | | UTT | |
| 1145 | 1145-1163 | UGGUGACAGAUCACGGCU | 986 | GAGCCGUGAUCUGUCACC | 987 |
| | | CTT | | ATT | |
| 1147 | 1147-1165 | GUGACAGAUCACGGCUCG | 988 | ACGAGCCGUGAUCUGUCA | 989 |
| | | UTT | | CTT | |
| 1148 | 1148-1166 | UGACAGAUCACGGCUCGU | 990 | CACGAGCCGUGAUCUGUC | 991 |
| | | GTT | | ATT | |
| 1149 | 1149-1167 | GACAGAUCACGGCUCGUG | 992 | GCACGAGCCGUGAUCUGU | 993 |
| | | CTT | | CTT | |
| 1150 | 1150-1168 | ACAGAUCACGGCUCGUGC | 994 | CGCACGAGCCGUGAUCUG | 995 |
| | | GTT | | UTT | |
| 1151 | 1151-1169 | CAGAUCACGGCUCGUGCG | 996 | ACGCACGAGCCGUGAUCU | 997 |
| | | UTT | | GTT | |
| 1152 | 1152-1170 | AGAUCACGGCUCGUGCGU | 998 | GACGCACGAGCCGUGAUC | 999 |
| | | CTT | | UTT | |
| 1153 | 1153-1171 | GAUCACGGCUCGUGCGUC | 1000 | GGACGCACGAGCCGUGAU | 1001 |
| | | CTT | | CTT | |
| 1154 | 1154-1172 | AUCACGGCUCGUGCGUCC | 1002 | CGGACGCACGAGCCGUGA | 1003 |
| | | GTT | | UTT | |
| 1155 | 1155-1173 | UCACGGCUCGUGCGUCCG | 1004 | UCGGACGCACGAGCCGUG | 1005 |
| | | ATT | | ATT | |
| 1156 | 1156-1174 | CACGGCUCGUGCGUCCGA | 1006 | CUCGGACGCACGAGCCGU | 1007 |
| | | GTT | | GTT | |
| 1157 | 1157-1175 | ACGGCUCGUGCGUCCGAG | 1008 | GCUCGGACGCACGAGCCG | 1009 |
| | | CTT | | UTT | |
| 1160 | 1160-1178 | GCUCGUGCGUCCGAGCCU | 1010 | CAGGCUCGGACGCACGAG | 1011 |
| | | GTT | | CTT | |
| 1200 | 1200-1218 | GGAGGAAGACGGCGUCCG | 1012 | GCGGACGCCGUCUUCCUC | 1013 |
| | | CTT | | CTT | |
| 1201 | 1201-1219 | GAGGAAGACGGCGUCCGC | 1014 | UGCGGACGCCGUCUUCCU | 1015 |
| | | ATT | | CTT | |
| 1203 | 1203-1221 | GGAAGACGGCGUCCGCAA | 1016 | CUUGCGGACGCCGUCUUC | 1017 |
| | | GTT | | CTT | |
| 1204 | 1204-1222 | GAAGACGGCGUCCGCAAG | 1018 | ACUUGCGGACGCCGUCUU | 1019 |
| | | UTT | | CTT | |
| 1205 | 1205-1223 | AAGACGGCGUCCGCAAGU | 1020 | CACUUGCGGACGCCGUCU | 1021 |
| | | GTT | | UTT | |
| 1207 | 1207-1225 | GACGGCGUCCGCAAGUGU | 1022 | UACACUUGCGGACGCCGU | 1023 |
| | | ATT | | CTT | |
| 1208 | 1208-1226 | ACGGCGUCCGCAAGUGUA | 1024 | UUACACUUGCGGACGCCG | 1025 |
| | | ATT | | UTT | |
| 1211 | 1211-1229 | GCGUCCGCAAGUGUAAGA | 1026 | UUCUUACACUUGCGGACG | 1027 |
| | | ATT | | CTT | |
| 1212 | 1212-1230 | CGUCCGCAAGUGUAAGAA | 1028 | CUUCUUACACUUGCGGAC | 1029 |
| | | GTT | | GTT | |
| 1213 | 1213-1231 | GUCCGCAAGUGUAAGAA | 1030 | ACUUCUUACACUUGCGGA | 1031 |
| | | GUTT | | CTT | |
| 1214 | 1214-1232 | UCCGCAAGUGUAAGAAG | 1032 | CACUUCUUACACUUGCGG | 1033 |
| | | UGTT | | ATT | |
| 1215 | 1215-1233 | CCGCAAGUGUAAGAAGU | 1034 | GCACUUCUUACACUUGCG | 1035 |
| | | GCTT | | GTT | |
| 1216 | 1216-1234 | CGCAAGUGUAAGAAGUG | 1036 | CGCACUUCUUACACUUGC | 1037 |
| | | CGTT | | GTT | |
| 1217 | 1217-1235 | GCAAGUGUAAGAAGUGC | 1038 | UCGCACUUCUUACACUUG | 1039 |
| | | GATT | | CTT | |
| 1219 | 1219-1237 | AAGUGUAAGAAGUGCGA | 1040 | CUUCGCACUUCUUACACU | 1041 |
| | | AGTT | | UTT | |
| 1220 | 1220-1238 | AGUGUAAGAAGUGCGAA | 1042 | CCUUCGCACUUCUUACAC | 1043 |
| | | GGTT | | UTT | |
| 1221 | 1221-1239 | GUGUAAGAAGUGCGAAG | 1044 | CCCUUCGCACUUCUUACA | 1045 |
| | | GGTT | | CTT | |
| 1222 | 1222-1240 | UGUAAGAAGUGCGAAGG | 1046 | GCCCUUCGCACUUCUUAC | 1047 |
| | | GCTT | | ATT | |
| 1223 | 1223-1241 | GUAAGAAGUGCGAAGGG | 1048 | GGCCCUUCGCACUUCUUA | 1049 |
| | | CCTT | | CTT | |
| 1224 | 1224-1242 | UAAGAAGUGCGAAGGGC | 1050 | AGGCCCUUCGCACUUCUU | 1051 |
| | | CUTT | | ATT | |
| 1225 | 1225-1243 | AAGAAGUGCGAAGGGCC | 1052 | AAGGCCCUUCGCACUUCU | 1053 |
| | | UUTT | | UTT | |
| 1226 | 1226-1244 | AGAAGUGCGAAGGGCCU | 1054 | CAAGGCCCUUCGCACUUC | 1055 |
| | | UGTT | | UTT | |
| 1229 | 1229-1247 | AGUGCGAAGGGCCUUGCC | 1056 | CGGCAAGGCCCUUCGCAC | 1057 |
| | | GTT | | UTT | |
| 1230 | 1230-1248 | GUGCGAAGGGCCUUGCCG | 1058 | GCGGCAAGGCCCUUCGCA | 1059 |
| | | CTT | | CTT | |
| 1231 | 1231-1249 | UGCGAAGGGCCUUGCCGC | 1060 | UGCGGCAAGGCCCUUCGC | 1061 |
| | | ATT | | ATT | |
| 1232 | 1232-1250 | GCGAAGGGCCUUGCCGCA | 1062 | UUGCGGCAAGGCCCUUCG | 1063 |
| | | ATT | | CTT | |
| 1233 | 1233-1251 | CGAAGGGCCUUGCCGCAA | 1064 | UUUGCGGCAAGGCCCUUC | 1065 |
| | | ATT | | GTT | |
| 1235 | 1235-1253 | AAGGGCCUUGCCGCAAAG | 1066 | ACUUUGCGGCAAGGCCCU | 1067 |
| | | UTT | | UTT | |
| 1236 | 1236-1254 | AGGGCCUUGCCGCAAAGU | 1068 | CACUUUGCGGCAAGGCCC | 1069 |
| | | GTT | | UTT | |
| 1237 | 1237-1255 | GGGCCUUGCCGCAAAGUG | 1070 | ACACUUUGCGGCAAGGCC | 1071 |
| | | UTT | | CTT | |
| 1238 | 1238-1256 | GGCCUUGCCGCAAAGUGU | 1072 | CACACUUUGCGGCAAGGC | 1073 |
| | | GTT | | CTT | |
| 1239 | 1239-1257 | GCCUUGCCGCAAAGUGUG | 1074 | ACACACUUUGCGGCAAGG | 1075 |
| | | UTT | | CTT | |
| 1241 | 1241-1259 | CUUGCCGCAAAGUGUGUA | 1076 | UUACACACUUUGCGGCAA | 1077 |
| | | ATT | | GTT | |
| 1261 | 1261-1279 | GGAAUAGGUAUUGGUGA | 1078 | AUUCACCAAUACCUAUUC | 1079 |
| | | AUTT | | CTT | |
| 1262 | 1262-1280 | GAAUAGGUAUUGGUGAA | 1080 | AAUUCACCAAUACCUAUU | 1081 |
| | | UUTT | | CTT | |
| 1263 | 1263-1281 | AAUAGGUAUUGGUGAAU | 1082 | AAAUUCACCAAUACCUAU | 1083 |
| | | UUTT | | UTT | |
| 1264 | 1264-1282 | AUAGGUAUUGGUGAAUU | 1084 | UAAAUUCACCAAUACCUA | 1085 |
| | | UATT | | UTT | |
| 1266 | 1266-1284 | AGGUAUUGGUGAAUUUA | 1086 | UUUAAAUUCACCAAUACC | 1087 |
| | | AATT | | UTT | |
| 1267 | 1267-1285 | GGUAUUGGUGAAUUUAA | 1088 | CUUUAAAUUCACCAAUAC | 1089 |
| | | AGTT | | CTT | |
| 1289 | 1289-1307 | CACUCUCCAUAAAUGCUA | 1090 | GUAGCAUUUAUGGAGAG | 1091 |
| | | CTT | | UGTT | |
| 1313 | 1313-1331 | UUAAACACUUCAAAAACU | 1092 | CAGUUUUUGAAGUGUUU | 1093 |
| | | GTT | | AATT | |
| 1320 | 1320-1338 | CUUCAAAAACUGCACCUC | 1094 | GGAGGUGCAGUUUUUGA | 1095 |
| | | CTT | | AGTT | |
| 1321 | 1321-1339 | UUCAAAAACUGCACCUCC | 1096 | UGGAGGUGCAGUUUUUG | 1097 |
| | | ATT | | AATT | |
| 1322 | 1322-1340 | UCAAAAACUGCACCUCCA | 1098 | AUGGAGGUGCAGUUUUU | 1099 |
| | | UTT | | GATT | |
| 1323 | 1323-1341 | CAAAAACUGCACCUCCAU | 1100 | GAUGGAGGUGCAGUUUU | 1101 |
| | | CTT | | UGTT | |
| 1324 | 1324-1342 | AAAAACUGCACCUCCAUC | 1102 | UGAUGGAGGUGCAGUUU | 1103 |
| | | ATT | | UUTT | |
| 1328 | 1328-1346 | ACUGCACCUCCAUCAGUG | 1104 | CCACUGAUGGAGGUGCA | 1105 |
| | | GTT | | GUTT | |
| 1332 | 1332-1350 | CACCUCCAUCAGUGGCGA | 1106 | AUCGCCACUGAUGGAGG | 1107 |
| | | UTT | | UGTT | |
| 1333 | 1333-1351 | ACCUCCAUCAGUGGCGAU | 1108 | GAUCGCCACUGAUGGAG | 1109 |
| | | CTT | | GUTT | |
| 1335 | 1335-1353 | CUCCAUCAGUGGCGAUCU | 1110 | GAGAUCGCCACUGAUGG | 1111 |
| | | CTT | | AGTT | |
| 1338 | 1338-1356 | CAUCAGUGGCGAUCUCCA | 1112 | GUGGAGAUCGCCACUGA | 1113 |
| | | CTT | | UGTT | |
| 1344 | 1344-1362 | UGGCGAUCUCCACAUCCU | 1114 | CAGGAUGUGGAGAUCGC | 1115 |
| | | GTT | | CATT | |
| 1345 | 1345-1363 | GGCGAUCUCCACAUCCUG | 1116 | GCAGGAUGUGGAGAUCG | 1117 |
| | | CTT | | CCTT | |
| 1346 | 1346-1364 | GCGAUCUCCACAUCCUGC | 1118 | GGCAGGAUGUGGAGAUC | 1119 |
| | | CTT | | GCTT | |
| 1347 | 1347-1365 | CGAUCUCCACAUCCUGCC | 1120 | CGGCAGGAUGUGGAGAU | 1121 |
| | | GTT | | CGTT | |
| 1348 | 1348-1366 | GAUCUCCACAUCCUGCCG | 1122 | CCGGCAGGAUGUGGAGA | 1123 |
| | | GTT | | UCTT | |
| 1353 | 1353-1371 | CCACAUCCUGCCGGUGGC | 1124 | UGCCACCGGCAGGAUGUG | 1125 |
| | | ATT | | GTT | |
| 1354 | 1354-1372 | CACAUCCUGCCGGUGGCA | 1126 | AUGCCACCGGCAGGAUGU | 1127 |
| | | UTT | | GTT | |
| 1355 | 1355-1373 | ACAUCCUGCCGGUGGCAU | 1128 | AAUGCCACCGGCAGGAUG | 1129 |
| | | UTT | | UTT | |
| 1357 | 1357-1375 | AUCCUGCCGGUGGCAUUU | 1130 | UAAAUGCCACCGGCAGGA | 1131 |
| | | ATT | | UTT | |
| 1360 | 1360-1378 | CUGCCGGUGGCAUUUAGG | 1132 | CCCUAAAUGCCACCGGCA | 1133 |
| | | GTT | | GTT | |
| 1361 | 1361-1379 | UGCCGGUGGCAUUUAGG | 1134 | CCCCUAAAUGCCACCGGC | 113 |
| | | GGTT | | ATT | 5 |
| 1362 | 1362-1380 | GCCGGUGGCAUUUAGGG | 1136 | ACCCCUAAAUGCCACCGG | 1137 |
| | | GUTT | | CTT | |
| 1363 | 1363-1381 | CCGGUGGCAUUUAGGGG | 1138 | CACCCCUAAAUGCCACCG | 1139 |
| | | UGTT | | GTT | |
| 1366 | 1366-1384 | GUGGCAUUUAGGGGUGA | 1140 | AGUCACCCCUAAAUGCCA | 1141 |
| | | CUTT | | CTT | |
| 1369 | 1369-1387 | GCAUUUAGGGGUGACUCC | 1142 | AGGAGUCACCCCUAAAUG | 1143 |
| | | UTT | | CTT | |
| 1370 | 1370-1388 | CAUUUAGGGGUGACUCCU | 1144 | AAGGAGUCACCCCUAAAU | 1145 |
| | | UTT | | GTT | |
| 1371 | 1371-1389 | AUUUAGGGGUGACUCCU | 1146 | GAAGGAGUCACCCCUAAA | 1147 |
| | | UCTT | | UTT | |
| 1372 | 1372-1390 | UUUAGGGGUGACUCCUUC | 1148 | UGAAGGAGUCACCCCUAA | 1149 |
| | | ATT | | ATT | |
| 1373 | 1373-1391 | UUAGGGGUGACUCCUUCA | 1150 | GUGAAGGAGUCACCCCUA | 1151 |
| | | CTT | | ATT | |
| 1374 | 1374-1392 | UAGGGGUGACUCCUUCAC | 1152 | UGUGAAGGAGUCACCCCU | 1153 |
| | | ATT | | ATT | |
| 1404 | 1404-1422 | UCUGGAUCCACAGGAACU | 1154 | CAGUUCCUGUGGAUCCAG | 1155 |
| | | GTT | | ATT | |
| 1408 | 1408-1426 | GAUCCACAGGAACUGGAU | 1156 | UAUCCAGUUCCUGUGGA | 1157 |
| | | ATT | | UCTT | |
| 1409 | 1409-1427 | AUCCACAGGAACUGGAUA | 1158 | AUAUCCAGUUCCUGUGG | 1159 |
| | | UTT | | AUTT | |
| 1411 | 1411-1429 | CCACAGGAACUGGAUAUU | 1160 | GAAUAUCCAGUUCCUGU | 1161 |
| | | CTT | | GGTT | |
| 1412 | 1412-1430 | CACAGGAACUGGAUAUUC | 1162 | AGAAUAUCCAGUUCCUG | 1163 |
| | | UTT | | UGTT | |
| 1419 | 1419-1437 | ACUGGAUAUUCUGAAAA | 1164 | GGUUUUCAGAAUAUCCA | 1165 |
| | | CCTT | | GUTT | |
| 1426 | 1426-1444 | AUUCUGAAAACCGUAAA | 1166 | CCUUUACGGUUUUCAGA | 1167 |
| | | GGTT | | AUTT | |
| 1427 | 1427-1445 | UUCUGAAAACCGUAAAG | 1168 | UCCUUUACGGUUUUCAG | 1169 |
| | | GATT | | AATT | |
| 1430 | 1430-1448 | UGAAAACCGUAAAGGAA | 1170 | AUUUCCUUUACGGUUUU | 1171 |
| | | AUTT | | CATT | |
| 1431 | 1431-1449 | GAAAACCGUAAAGGAAA | 1172 | GAUUUCCUUUACGGUUU | 1173 |
| | | UCTT | | UCTT | |

TABLE 5
AR Target Sequences

| | | | SEQ | | | |
|---|---|---|---|---|---|---|
| | | | ID | | | |
| ID | Code | Target Sequence | NO: | NM_000044.3 | Exon | Species |
| XD- | 17 | CAAAGGUUCUCUGCUAGACGAC | 1174 | 1987-2005 | 1 | h |
| 01817K1 | | A | | | | |
| XD- | 27 | UCUGGGUGUCACUAUGGAGCUC | 1175 | 2819-2837 | 2 | h |
| 01827K1 | | U | | | | |
| XD- | 28 | CUGGGUGUCACUAUGGAGCUCU | 1176 | 2820-2838 | 2 | h |
| 01828K1 | | C | | | | |
| XD- | 29 | GGGUGUCACUAUGGAGCUCUCA | 1177 | 2822-2840 | 2 | h |
| 01829K1 | | C | | | | |
| XD- | 21 | UACUACAACUUUCCACUGGCUCU | 1178 | 2207-2225 | 1 | h |
| 01821K1 | | | | | | |
| XD- | 25 | AAGCUUCUGGGUGUCACUAUGG | 1179 | 2814-2832 | 2 | h, m |
| 01825K1 | | A | | | | |
| XD- | 26 | CUUCUGGGUGUCACUAUGGAGCU | 1180 | 2817-2835 | 2 | h |
| 01826K1 | | | | | | |

TABLE 6
β-catenin Target Sequences

| | Generic | | | | | |
|---|---|---|---|---|---|---|
| R# | name | Gene | Target sequences | | | |
| R-1146 | 1797mfm | CTNNB1 | CUGUUGGAUUGAU | SEQ ID | UUUCGAAUCAAUCCA | SEQ ID |
| | | | UCGAAAUU | NO. | ACAGUU | NO: |
| | | | | 1181 | | 1182 |
| R-1147 | 1870mfm | CTNNB1 | ACGACUAGUUCAG | SEQ ID | AAGCAACUGAACUAG | SEQ ID |
| | | | UUGCUUUU | NO: | UCGUUU | NO. |
| | | | | 1183 | | 1184 |

TABLE 7
PIK3CA* and PIK3CB* Target Sequences

| Gene | Gene | | | SEQ ID |
|---|---|---|---|---|
| Symbol | ID | Name | Target Sequences (97-mer) | NO: |
| PIK3CA | 5290 | PIK3CA_1746 | TGCTGTTGACAGTGAGCGCCAGCTCAAAGCAATT | 1185 |
| | | | TCTACATAGTGAAGCCACAGATGTATGTAGAAAT | |
| | | | TGCTTTGAGCTGTTGCCTACTGCCTCGGA | |
| PIK3CA | 5290 | PIK3CA_2328 | TGCTGTTGACAGTGAGCGAAAGGATGAAACACAA | 1186 |
| | | | AAGGTATAGTGAAGCCACAGATGTATACCTTTTGT | |
| | | | GTTTCATCCTTCTGCCTACTGCCTCGGA | |
| PIK3CA | 5290 | PIK3CA_2522 | TGCTGTTGACAGTGAGCGCCATGTCAGAGTTACTG | 1187 |
| | | | TTTCATAGTGAAGCCACAGATGTATGAAACAGTA | |
| | | | ACTCTGACATGATGCCTACTGCCTCGGA | |
| PIK3CA | 5290 | PIK3CA_3555 | TGCTGTTGACAGTGAGCGCAACTAGTTCATTTCAA | 1188 |
| | | | AATTATAGTGAAGCCACAGATGTATAATTTTGAA | |
| | | | ATGAACTAGTTTTGCCTACTGCCTCGGA | |
| PIK3CA | 5290 | PIK3CA_3484 | TGCTGTTGACAGTGAGCGCACAGCAAGAACAGAA | 1189 |
| | | | ATAAAATAGTGAAGCCACAGATGTATTTTATTTCT | |
| | | | GTTCTTGCTGTATGCCTACTGCCTCGGA | |
| PIK3CB | 5291 | PIK3CB_862 | TGCTGTTGACAGTGAGCGACAAGATCAAGAAAAT | 1190 |
| | | | GTATGATAGTGAAGCCACAGATGTATCATACATTT | |
| | | | TCTTGATCTTGCTGCCTACTGCCTCGGA | |
| PIK3CB | 5291 | PIK3CB_183 | TGCTGTTGACAGTGAGCGCAGCAAGTTCACAATT | 1191 |
| | | | ACCCAATAGTGAAGCCACAGATGTATTGGGTAAT | |
| | | | TGTGAACTTGCTTTGCCTACTGCCTCGGA | |
| PIK3CB | 5291 | PIK3CB_1520 | TGCTGTTGACAGTGAGCGCCCCTTCGATAAGATTA | 1192 |
| | | | TTGAATAGTGAAGCCACAGATGTATTCAATAATCT | |
| | | | TATCGAAGGGATGCCTACTGCCTCGGA | |
| PIK3CB | 5291 | PIK3CB_272 | TGCTGTTGACAGTGAGCGAGAGCTTGAAGATGAA | 1193 |
| | | | ACACGATAGTGAAGCCACAGATGTATCGTGTTTC | |
| | | | ATCTTCAAGCTCCTGCCTACTGCCTCGGA | |
| PIK3CB | 5291 | PIK3CB_948 | TGCTGTTGACAGTGAGCGACACCAAAGAAAACAC | 1194 |
| | | | GAATTATAGTGAAGCCACAGATGTATAATTCGTG | |
| | | | TTTTCTTTGGTGGTGCCTACTGCCTCGGA | |

*Species is Homo sapiens.

TABLE 8
PIK3CA and PIK3CB siRNA Sequences

| | | | | SEQ | | SEQ |
|---|---|---|---|---|---|---|
| Gene | Gene | | | ID | | ID |
| Symbol | ID | Name | siRNA Guide | NO: | siRNA passenger | NO: |
| PIK3CA | 5290 | PIK3CA_1746 | UGUAGAAAUUGCUU | 1195 | AGCUCAAAGCAAUU | 1196 |
| | | | UGAGCUGU | | UCUACAUA | |
| PIK3CA | 5290 | PIK3CA_2328 | UACCUUUUGUGUUU | 1197 | AGGAUGAAACACAA | 1198 |
| | | | CAUCCUUC | | AAGGUAUA | |
| PIK3CA | 5290 | PIK3CA_2522 | UGAAACAGUAACUC | 1199 | AUGUCAGAGUUACU | 1200 |
| | | | UGACAUGA | | GUUUCAUA | |
| PIK3CA | 5290 | PIK3CA_3555 | UAAUUUUGAAAUGA | 1201 | ACUAGUUCAUUUCA | 1202 |
| | | | ACUAGUUU | | AAAUUAUA | |
| PIK3CA | 5290 | PIK3CA_3484 | UUUUAUUUCUGUUC | 1203 | CAGCAAGAACAGAA | 1204 |
| | | | UUGCUGUA | | AUAAAAUA | |
| PIK3CB | 5291 | PIK3CB_862 | UCAUACAUUUUCUU | 1205 | AAGAUCAAGAAAAU | 1206 |
| | | | GAUCUUGC | | GUAUGAUA | |
| PIK3CB | 5291 | PIK3CB_183 | UUGGGUAAUUGUGA | 1207 | GCAAGUUCACAAUU | 1208 |
| | | | ACUUGCUU | | ACCCAAUA | |
| PIK3CB | 5291 | PIK3CB_1520 | UUCAAUAAUCUUAU | 1209 | CCUUCGAUAAGAUU | 1210 |
| | | | CGAAGGGA | | AUUGAAUA | |
| PIK3CB | 5291 | PIK3CB_272 | UCGUGUUUCAUCUU | 1211 | AGCUUGAAGAUGAA | 1212 |
| | | | CAAGCUCC | | ACACGAUA | |
| PIK3CB | 5291 | PIK3CB_948 | UAAUUCGUGUUUUC | 1213 | ACCAAAGAAAACAC | 1214 |
| | | | UUUGGUGG | | GAAUUAUA | |

TABLE 9
Additional polynucleic acid molecule sequences

| | Base | | SEQ | | SEQ |
|---|---|---|---|---|---|
| | start | | ID | | ID |
| | position | Guide strand | NO: | Passenger strand | NO: |
| EGFR | 333 | ACUCGUGCCUUGGCAA | 1215 | AGUUUGCCAAGGCACGA | 1216 |
| R1246 | | ACUUU | | GUUU | |
| EGFR | 333 | ACUCGUGCCUUGGCAA | 1217 | AGUUUGCCAAGGCACGA | 1218 |
| R1195 | | ACUUU | | GUUU | |
| EGFR | 333 | ACUCGUGCCUUGGCAA | 1219 | AGUUUGCCAAGGCACGA | 1220 |
| R1449 | | ACUUU | | GUUU | |
| KRAS | 237 | UGAAUUAGCUGUAUC | 1221 | TGACGAUACAGCUAAUU | 1222 |
| R1450 | | GUCAUU | | CAUU | |
| KRAS | 237 | UGAAUUAGCUGUAUC | 1223 | UGACGAUACAGCUAAUU | 1224 |
| R1443 | | GUCAUU | | CAUU | |
| KRAS | 237 | UGAAUUAGCUGUAUC | 1225 | UGACGAUACAGCUAAUU | 1226 |
| R1194 | | GUCAUU | | CAUU | |
| CTNNB1 | 1248 | UAAGUAUAGGUCCUCA | 1227 | UAAUGAGGACCUAUACU | 1228 |
| R1442 | | UUAUU | | UAUU | |
| CTNNB1 | 1797 | TUUCGAAUCAAUCCAA | 1229 | CUGUUGGAUUGAUUCGA | 1230 |
| R1404 | | CAGUU | | AAUU | |
| CTNNB1 | 1797 | UUUCGAAUCAAUCCAA | 1231 | CUGUUGGAUUGAUUCGA | 1232 |
| R1441 | | CAGUU | | AAUU | |
| CTNNB1 | 1797 | UUUCGAAUCAAUCCAA | 1233 | CUGUUGGAUUGAUUCGA | 1234 |
| R1523 | | CAGUU | | AAUU | |
| HPRT | 425 | AUAAAAUCUACAGUCA | 1235 | CUAUGACUGUAGAUUUU | 1236 |
| R1492 | | UAGUU | | AUUU | |
| HPRT | 425 | UUAAAAUCUACAGUCA | 1237 | CUAUGACUGUAGAUUUU | 1238 |
| R1526 | | UAGUU | | AAUU | |
| HPRT | 425 | UUAAAAUCUACAGUCA | 1239 | CUAUGACUGUAGAUUUU | 1240 |
| R1527 | | UAGUU | | AAUU | |
| AR | 2822 | GAGAGCUCCAUAGUGA | 1241 | GUGUCACUAUGGAGCUC | 1242 |
| R1245 | | CACUU | | UCUU | |

### Example 2. General Experimental Protocol

Stem-Loop qPCR Assay for Quantification of siRNA

Plasma samples are directly diluted in TE buffer. 50 mg tissue pieces are homogenized in 1 mL of Trizol using a FastPrep-24 tissue homogenizer (MP Biomedicals) and then diluted in TE buffer. Standard curves are generated by spiking siRNA into plasma or homogenized tissue from untreated animals and then serially diluting with TE buffer. The antisense strand of the siRNA is reverse transcribed using a TaqMan MicroRNA reverse transcription kit (Applied Biosystems) with 25 nM of a sequence-specific stem-loop RT primer. The cDNA from the RT step is utilized for real-time PCR using TaqMan Fast Advanced Master Mix (Applied Biosystems) with 1.5 μM of forward primer, 0.75 μM of reverse primer, and 0.2 μM of probe. Quantitative PCR reactions are performed using standard cycling conditions in a ViiA 7 Real-Time PCR System (Life Technologies). The Ct values are transformed into plasma or tissue concentrations using the linear equations derived from the standard curves.

Comparative qPCR Assay for Determination of mRNA Knockdown

Tissue samples are homogenized in Trizol as described above. Total RNA is isolated using RNeasy RNA isolation 96-well plates (Qiagen), then 500 ng RNA is reverse transcribed with a High Capacity RNA to cDNA kit (ThermoFisher). KRAS, EGFR, CTNNB1, and PPIB mRNAs are quantified by TaqMan qPCR analysis performed with a ViiA 7 Real-Time PCR System. The TaqMan primers and probes for EGFR and CTNNB1 are purchased from Applied Biosystems as pre-validated gene expression assays. PPIB (housekeeping gene) is used as an internal RNA loading control, with all TaqMan primers and probes for PPIB purchased from Applied Biosystems as pre-validated gene expression assays. Results are calculated by the comparative Ct method, where the difference between the target gene (KRAS, CTNNB1, or EGFR) Ct value and the PPIB Ct value (ΔCt) is calculated and then further normalized relative to the PBS control group by taking a second difference (ΔΔCt).

### Animals

All animal studies are conducted following protocols in accordance with the Institutional Animal Care and Use Committee (IACUC) at Explora BioLabs, which adhere to the regulations outlined in the USDA Animal Welfare Act as well as the “Guide for the Care and Use of Laboratory Animals” (National Research Council publication, 8th Ed., revised in 2011). All mice are obtained from either Charles River Laboratories or Harlan Laboratories.

### H358, HCC827, and Hep-3B2 1-7 Subcutaneous Flank Tumor Model

For the H358 subcutaneous flank tumor model, tumor cells are inoculated and tumors are established according to the following methods. Female NCr nu/nu mice are identified by ear-tag the day before cell injection. Mice are weighed prior to inoculation. H358 cells are cultured with 10% FBS/RPMI medium and harvested with 0.05% Trypsin and Cell Stripper (MediaTech). 5 million H358 cells in 0.05 ml Hank's Balanced Salt Solution (HBSS) with Matrigel (1:1) are injected subcutaneously (SC) into the upper right flank of each mouse. Tumor growth is monitored by tumor volume measurement using a digital caliper starting on day 7 after inoculation, and followed 2 times per week until average tumor volume reaches >100 & ≤300 mm³. Once tumors are to the desired volume (average from 100 to 300 mm³), animals are randomized and mice with very large or small tumors are culled. Mice are divided into the required groups and randomized by tumor volume. Mice are then treated as described in the individual experiments.

For the Hep3B orthotropic liver tumor model, tumor cells are inoculated and tumors are established according to the following methods. Female NCr nu/nu mice are identified by ear-tag the day before, mice will be anesthetized with isoflurane. The mice are then placed in a supine position on a water circulating heating pad to maintain body temperature. A small transverse incision below the sternum will be made to expose the liver. Cancer cells are slowly injected into the upper left lobe of the liver using a 28-gauge needle. The cells are injected at a 30-degree angle into the liver, so that a transparent bleb of cells can be seen through the liver capsule. Hep 3B2.1 7 cells are prepared by suspending in cold PBS (0.1-5×10⁶ cells) and mixing with diluted matrigel (30× in PBS). 30-50 ul of the cell/matrigel is inoculated. After injection, a small piece of sterile gauze is placed on the injection site, and light pressure was applied for 1 min to prevent bleeding. The abdomen is then closed with a 6-0 silk suture. After tumor cell implantation, animals are kept in a warm cage, observed for 1-2 h, and subsequently returned to the animal room after full recovery from the anesthesia. 7-10 days after tumor implantation animals are randomized, divided into the required groups and then treated as described in the individual experiments.

### LNCap Subcutaneous Flank Tumor Model

LNCaP cells (ATCC® CRLA740™) are grown in RPMI+10% FBS supplemented with non-essential amino acids and sodium pyruvate to a confluency of about 80%. Cells are mixed 1:1 with matrigel and 5-7*106 cells injected subcutaneously into male SCID mice (6-8 weeks). Tumors are usually developed within 3-5 weeks to a size of 100-350 mm³. Animals bearing tumors within this range are randomized and treated with ASCs by injections into the tail vein. For PD studies animals are sacrificed 96 hours after injection and organ fragments harvested, weighted, and frozen in liquid nitrogen. For RNA isolation, organ samples are homogenized in Trizol and RNA prepared using a Qiagen RNeasy 96 Plus kit following the instructions by the manufacturer. RNA concentrations are determined spectroscopically. RNAs are converted into cDNAs by reverse transcription and expression of specific targets quantified by qPCR using the ΔΔCT method and validated Taqman assays (Thermofisher). Samples are standardize to the expression levels of PPIB.

### Peptide Synthesis

Peptides are synthesized on solid phase using standard Fmoc chemistry. Both peptides have cysteine at the N-terminus and the cleaved peptides are purified by HPLC and confirmed by mass spectroscopy. INF7 peptide is as illustrated SEQ ID NO: 2055. Melittin peptide is as illustrated SEQ ID NO: 2060.

### Anti-EGFR Antibody

Anti-EGFR antibody is a fully human IgG1κ monoclonal antibody directed against the human epidermal growth factor receptor (EGFR). It is produced in the Chinese Hamster Ovary cell line DJT33, which has been derived from the CHO cell line CHO-KlSV by transfection with a GS vector carrying the antibody genes derived from a human anti-EGFR antibody producing hybridoma cell line (2F8). Standard mammalian cell culture and purification technologies are employed in the manufacturing of anti-EGFR antibody.

The theoretical molecular weight (MW) of anti-EGFR antibody without glycans is 146.6 kDa. The experimental MW of the major glycosylated isoform of the antibody is 149 kDa as determined by mass spectrometry. Using SDS-PAGE under reducing conditions the MW of the light chain was found to be approximately 25 kDa and the MW of the heavy chain to be approximately 50 kDa. The heavy chains are connected to each other by two inter-chain disulfide bonds, and one light chain is attached to each heavy chain by a single inter-chain disulfide bond. The light chain has two intra-chain disulfide bonds and the heavy chain has four intra-chain disulfide bonds. The antibody is N-linked glycosylated at Asn305 of the heavy chain with glycans composed of N-acetyl-glucosamine, mannose, fucose and galactose. The predominant glycans present are fucosylated bi-antennary structures containing zero or one terminal galactose residue.

The charged isoform pattern of the IgG1κ antibody has been investigated using imaged capillary IEF, agarose IEF and analytical cation exchange HPLC. Multiple charged isoforms are found, with the main isoform having an isoelectric point of approximately 8.7.

The major mechanism of action of anti-EGFR antibody is a concentration dependent inhibition of EGF-induced EGFR phosphorylation in A431 cancer cells. Additionally, induction of antibody-dependent cell-mediated cytotoxicity (ADCC) at low antibody concentrations has been observed in pre-clinical cellular in vitro studies.

### Example 3: Synthesis, Purification and Analysis of Antibody-PEG-EGFR and Antibody-EGFR Conjugates

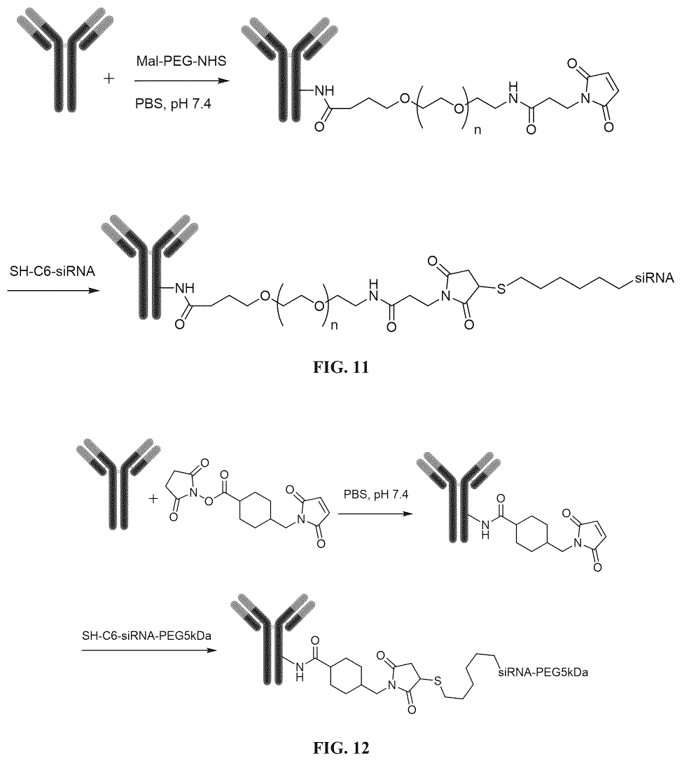

Conjugation scheme-1 for generating Antibody-EGFR conjugates is depicted in FIG. 11

Step 1: Antibody Conjugation with Maleimide-PEG-NHS Followed by SH-EGFR

Anti-EGFR antibody (EGFR-Ab) is exchanged with 1× Phosphate buffer (pH 7.4) and made up to 5 mg/ml concentration. To this solution, 2 equivalents of SMCC linker or maleimide-PEGxkDa-NHS (x=1, 5, 10, 20) is added and rotated for 4 hours at room temperature. Unreacted maleimide-PEG is removed by spin filtration using 50 kDa MWCO Amicon spin filters and PBS pH 7.4. The antibody-PEG-Mal conjugate is collected and transferred into a reaction vessel. SH-C6-EGFR (2 equivalents) is added at RT to the antibody-PEG-maleimide in PBS and rotated overnight. The reaction mixture is analyzed by analytical SAX column chromatography and conjugate along with unreacted antibody and siRNA is seen.

### Step 2: Purification

The crude reaction mixture is purified by AKTA explorer FPLC using anion exchange chromatography method-1. Fractions containing the antibody-PEG-EGFR conjugate ae pooled, concentrated and buffer exchanged with PBS, pH 7.4. Antibody siRNA conjugates with SMCC linker, PEG1 kDa, PEG5 kDa and PEG10 kDa are separated based on the siRNA loading.

### Step-3: Analysis of the Purified Conjugate

The isolated conjugate ias characterized by either mass spec or SDS-PAGE. The purity of the conjugate is assessed by analytical HPLC using either anion exchange chromatography method-2 or anion exchange chromatography method-3.

### Anion Exchange Chromatography Method-1

1. Column: Tosoh Bioscience, TSKGel SuperQ-5PW, 21.5 mm ID×15 cm, 13 μm
2. Solvent A: 20 mM TRIS buffer, pH 8.0; Solvent B: 20 mM TRIS, 1.5 M NaCl, pH 8.0; Flow Rate: 6.0 ml/min
3. Gradient:

| a. | % A | % B | Column Volume |
|---|---|---|---|
| b. | 100 | 0 | 1.00 |
| c. | 60 | 40 | 18.00 |
| d. | 40 | 60 | 2.00 |
| e. | 40 | 60 | 5.00 |
| f. | 0 | 100 | 2.00 |
| g. | 100 | 0 | 2.00 |

### Anion Exchange Chromatography Method-2

1. Column: Thermo Scientific, ProPac™ SAX-10, Bio LC™, 4×250 mm
2. Solvent A: 80% 10 mM TRIS pH 8, 20% ethanol; Solvent B: 80% 10 mM TRIS pH 8, 20% ethanol, 1.5 M NaCl; Flow Rate: 1.0 ml/min
3. Gradient:

| a. | Time % | % A | % B |
|---|---|---|---|
| b. | 0.0 | 90 | 10 |
| c. | 3.00 | 90 | 10 |
| d. | 11.00 | 40 | 60 |
| e. | 13.00 | 40 | 60 |
| f. | 15.00 | 90 | 10 |
| g. | 20.00 | 90 | 10 |

### Anion Exchange Chromatography Method-3

1. Column: Thermo Scientific, ProPac™ SAX-10, Bio LC™, 4×250 mm
2. Solvent A: 80% 10 mM TRIS pH 8, 20% ethanol; Solvent B: 80% 10 mM TRIS pH 8, 20% ethanol, 1.5 M NaCl
3. Flow Rate: 0.75 ml/min
4. Gradient:

| a. | Time | % A | % B |
|---|---|---|---|
| b. | 0.0 | 90 | 10 |
| c. | 3.00 | 90 | 10 |
| d. | 11.00 | 40 | 60 |
| e. | 23.00 | 40 | 60 |
| f. | 25.00 | 90 | 10 |
| g. | 30.00 | 90 | 10 |

### Example 4: Synthesis, Purification and Analysis of Antibody-siRNA-PEG Conjugates

Conjugation scheme-2 for generating antibody-siRNA-PEG conjugates is depicted in FIG. 12.

Step 1: Antibody Conjugation with SMCC Linker Followed by SH-KRAS-PEG5 kDa

Anti-EGFR antibody is exchanged with 1× Phosphate buffer (pH 7.4) and made up to 5 mg/ml concentration. To this solution, 2 equivalents of SMCC linker (succinimidyl 4-(N-maleimidomethyl)cyclohexane-1-carboxylate) is added and rotated for 4 hours at room temperature. Unreacted SMCC linker is removed by spin filtration using 50 kDa MWCO Amicon spin filters and PBS buffer pH 7.4. The retentate is collected and 2 equivalents of SH-C6-KRAS-PEG5 kDa is added at RT and rotated overnight. The reaction mixture is analyzed by analytical SAX column chromatography.

### Step 2: Purification

The crude reaction mixture is purified by AKTA explorer FPLC using anion exchange chromatography method-1. Fractions containing the antibody-KRAS-PEG conjugate are pooled, concentrated and buffer exchanged with PBS, pH 7.4.

### Step-3: Analysis of the Purified Conjugate

The isolated conjugate is characterized by either mass spec or SDS-PAGE. The purity of the conjugate is assessed by analytical HPLC using anion exchange chromatography method-3 (described in example 1).

### Example 5: Synthesis, Purification and Analysis of Antibody-S—S-siRNA-PEG Conjugates

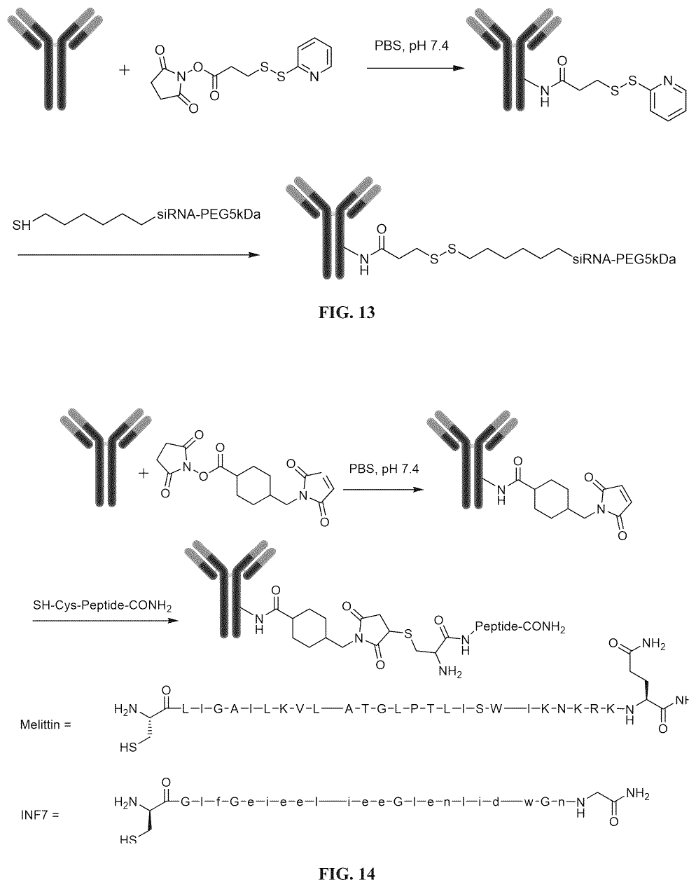

Conjugation scheme-3 for generating antibody-S—S-siRNA-PEG conjugates is depicted in FIG. 13.

Step 1: Antibody Conjugation with SPDP Linker Followed by SH-siRNA-PEG5 kDa

Anti-EGFR antibody is exchanged with 1× Phosphate buffer (pH 7.4) and made up to 5 mg/ml concentration. To this solution, 2 equivalents of SPDP linker (succinimidyl 3-(2-pyridyldithio)propionate) is added and rotated for 4 hours at room temperature. Unreacted SPDP linker is removed by spin filtration using 50 kDa MWCO Amicon spin filters and pH 7.4 PBS buffer. The retentate is collected and 2 equivalents of SH-C6-siRNA-PEG5 kDa is added at room temperature and rotated overnight. The reaction mixture is analyzed by analytical SAX column chromatography and conjugate along with unreacted antibody determined.

### Step 2: Purification

The crude reaction mixture is purified by AKTA explorer FPLC using anion exchange chromatography method-1. Fractions containing the antibody-PEG-siRNA conjugate are pooled, concentrated and buffer exchanged with PBS, pH 7.4.

### Step-3: Analysis of the Purified Conjugate

The isolated conjugate is characterized by either mass spec or SDS-PAGE. The purity of the conjugate is assessed by analytical HPLC using anion exchange chromatography method-2.

### Example 6: Synthesis, Purification and Analysis of Antibody-SMCC-Endosomal Escape Peptide Conjugates (SEQ ID NOS 1248 and 1277 Disclosed Below, Respectively, in Order of Appearance)

Conjugation scheme-4 for generating antibody-SMCC-Endosomal escape peptide conjugates is depicted in FIG. 14.

Step 1: Antibody Conjugation with SMCC Linker or Maleimide-PEG-NHS Followed by SH-Cys-Peptide-CONH₂

Anti-EGFR antibody is exchanged with 1× Phosphate buffer (pH 7.4) and made up to 10 mg/ml concentration. To this solution, 3 equivalents of SMCC linker (succinimidyl 4-(N-maleimidomethyl)cyclohexane-1-carboxylate) or maleimide-PEG1 kDa-NHS is added and rotated for 1.5 hours at room temperature. Unreacted SMCC linker or PEG linker is removed by spin filtration using 50 kDa MWCO Amicon spin filters and PBS buffer pH 7.4 (25 mM MES pH=6.1 for Melittin conjugates). The retentate is collected and 3 equivalents of SH-Cys-Peptide-CONH₂ is added at RT and rotated overnight. The reaction mixture is then purified by either HIC chromatography or cation exchange chromatography to isolate the anti-EGFR antibody-Peptide or anti-EGFR antibody-PEGik-Peptide.

### Step 2: Purification

The crude reaction mixture is purified by AKTA explorer FPLC using either hydrophobic interaction chromatography (HIC) method-1 or cation exchange chromatography method-1. Fractions containing the antibody-peptide conjugates are pooled, concentrated and buffer exchanged with PBS, pH 7.4 (10 mM Acetate pH=6.0 for Melittin conjugates).

### Step-3: Analysis of the Purified Conjugate

The isolated conjugate is characterized by either mass spec or SDS-PAGE. Purity and peptide loading is assessed by analytical HPLC using either HIC method-2 or cation exchange chromatography method-2.

### Cation Exchange Chromatography Method-1

1. Column: GE Healthcare HiPrep SP HP 16/10
2. Solvent A: 50 mM MES pH=6.0; Solvent B: 50 mM MES+0.5M NaCl pH=6.0; Flow Rate: 2.0 ml/min
3. Gradient:

| a. | % A | % B | Column Volume |
|---|---|---|---|
| b. | 100 | 0 | 0.1 |
| c. | 100 | 0 | Flush loop 12 ml |
| d. | 100 | 0 | 2.5 |
| e. | 0 | 100 | 15 |
| f. | 0 | 100 | 5 |
| g. | 100 | 0 | 0.5 |
| h. | 100 | 0 | 5 |

### Cation Exchange Chromatography Method-2

1. Column: Thermo Scientific, MAbPac™ SCX-10, Bio LC™, 4×250 mm (product #074625)
2. Solvent A: 20 mM MES pH=5.5; Solvent B: 20 mM MES+0.3 M NaCl pH=5.5; Flow Rate: 0.5 ml/min
3. Gradient:

| a. | Time | % A | % B |
|---|---|---|---|
| b. | 0.0 | 100 | 0 |
| c. | 5 | 100 | 0 |
| d. | 50 | 0 | 100 |
| e. | 80 | 0 | 100 |
| f. | 85 | 100 | 0 |
| g. | 90 | 100 | 0 |

### Hydrophobic Interaction Chromatography Method-1 (HIC Method-1)

1. Column: GE Healthcare Butyl Sepharose High Performance (17-5432-02) 200 ml
2. Solvent A: 50 mM Sodium Phosphate+0.8M ammonium sulfate (pH=7.0); Solvent B: 80% 50 mM Sodium Phosphate (pH=7.0), 20% IPA; Flow Rate: 3.0 ml/min
3. Gradient:

| a. | % A | % B | Column Volume |
|---|---|---|---|
| b. | 100 | 0 | 0.1 |
| c. | 0 | 100 | 3 |
| d. | 0 | 100 | 1.35 |
| e. | 100 | 0 | 0.1 |
| f. | 100 | 0 | 0.5 |

### Hydrophobic Interaction Chromatography Method-2 (HIC Method-2)

1. Column: Tosoh Bioscience TSKgel Butyl-NPR 4.6 mm ID×10 cm 2.5 μm
2. Solvent A: 100 mM Sodium phosphate+1.8 M ammonium sulfate (pH=7.0); Solvent B: 80% 100 mM sodium phosphate (pH=7.0), 20% IPA; Flow Rate: 0.5 ml/min
3. Gradient:

| a. | Time | % A | % B |
|---|---|---|---|
| b. | 0 | 100 | 0 |
| c. | 3 | 50 | 50 |
| d. | 21 | 0 | 100 |
| e. | 23 | 0 | 100 |
| f. | 25 | 100 | 0 |

### Example 7: Synthesis, Purification and Analysis of EEP-Antibody-siRNA-PEG Conjugates

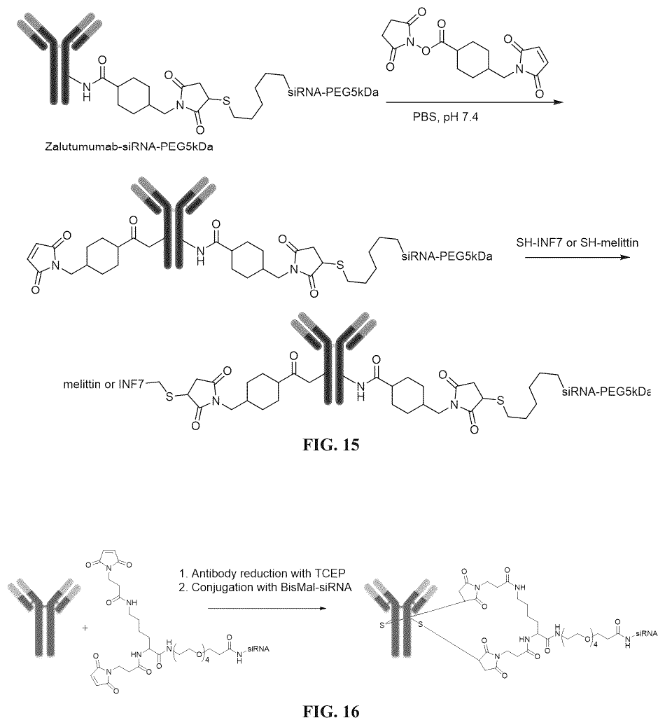

Conjugation scheme-5 for generating EEP-antibody-siRNA-PEG conjugates is depicted in FIG. 15.

### Step 1: Conjugation of PEG24 Linker Followed by SH-Cys-Peptide-CONH₂ to EGFR-Ab-siRNA-PEG

EGFR-Ab-siRNA-PEG5 kDa ugate with a siRNA loading of 1 is conjugated with 4 equivalents of PEG1k linker (succinimidyl 4-(N-maleimidomethyl)cyclohexane-1-carboxylate) in PBS, pH 7.4 buffer and rotated for 1.5 hours at room temperature. Unreacted PEG1k linker is removed by spin filtration using 50 kDa MWCO Amicon spin filters and PBS buffer pH 7.4. The retentate is collected and 4 equivalents of SH-Cys-Peptide-CONH₂ is added at RT and rotated overnight.

### Step 2: Purification

The reaction mixture is then purified by repeated spin filtration using PBS buffer pH7.4 and 50 kDa Amicon spin filters until the unreacted peptide is removed as monitored by HPLC. The product contains a mixture of conjugates with 0, 1, 2, 3 or more peptides conjugated to the antibody backbone.

### Step-3: Analysis of the Purified Conjugate

The isolated conjugate is characterized by either mass spec or SDS-PAGE. The purity and the peptide loading of the conjugate is assessed by analytical HPLC using either HIC method-2 or cation exchange chromatography method-2.

### Example 8. Tumor PK/PD Study

Female NCr nu/nu mice bearing subcutaneous flank H358 tumors will be dosed with EGFR antibody-siRNA-EEP conjugates at 0.5 mg/kg (based on siRNA). Multiple EEPs (endosomolytic moieties) will be used to determine the peptide sequence that demonstrates optimal endosomal escape, resulting in the best knockdown of the target gene relative to the control.

### Example 9. Formulation of an ABC Conjugate with Nanoparticles

An exemplary ABC conjugate is packaged into self-assembled nanoparticles using cyclodextrin polymers (10 kDa) and an excess of non-conjugated siRNAs (ED 40-60 nm, PDI 0.1-0.2). In these particles, the exemplary ABC conjugate maintains its ability to interact with the antibody target. The stability and target binding competency of the particles in circulation in vivo is regulated through modifications of the packaging siRNAs.

### Nanoparticle Formation

Nanoparticles are prepared at a final siRNA concentration of 1.6 mg/mL. siRNA containing CY5-siRNA at a ratio of 1:20 is first diluted to 2x final concentration in water. Cyclodextrin polymer (CDP) is diluted to 2x final concentration necessary to achieve a nitrogen to phosphorus ratio (N:P) of 3:1 in 10 mM phosphate buffer at neutral pH. CDP is added quickly to siRNA and is further mixed by pipetting. Particles are incubated for at least 15 minutes before dosing or analysis.

### In Vitro EGFR Binding

Nanoparticles containing various amount of the exemplary ABC conjugate are diluted into Fetal calf serum to a final concentration of 10 nM and are incubated for 1 h at RT with Protein G Dynabeads (Thermofisher) loaded with 150 nM of a purified EGFR-Fc protein (Sino Biological). Beads are washed twice with PBS containing 0.01% Tween 20 and 0.05% BSA before bead-bound nanoparticles are disrupted with water containing 0.01% Tween 20 and 100 μg/ml heparin. The amount of CY5-siRNA contained in the input, unbound fraction, washes and bead eluate is quantified by fluorescence using a TECAN Infinite M200 Pro (Excitation 635 nm; Emission 675 nm).

### Example 10. siRNA Synthesis

All siRNA single strands were fully assembled on solid phase using standard phospharamidite chemistry and purified using HPLC. Base, sugar and phosphate modifications that are well described in the field of RNAi were used to optimize the potency of the duplex and reduce immunogenicity.

The vinylphosphonate modified nucleotides (compounds 3, 15, 26, 27 and 28) used for examples 11-15 are shown in the table below

| Structure | Cmpd # |
|---|---|
| 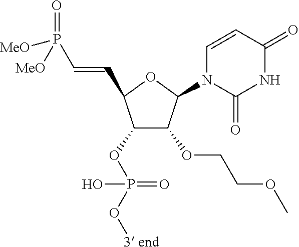 | 3 |
| 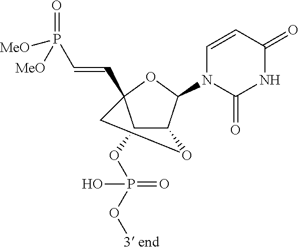 | 15 |
| 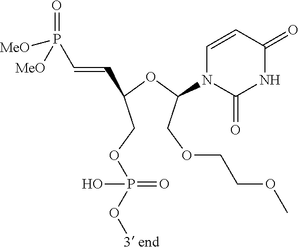 | 26 |
| 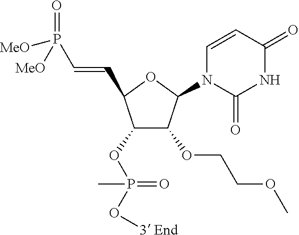 | 27 |
| 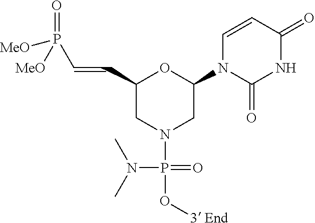 | 28 |
| 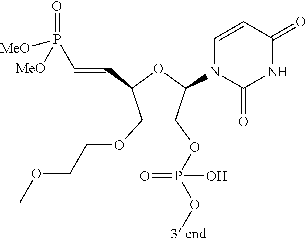 | 30 |
| 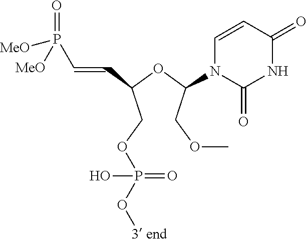 | 32 |

Compound 3 was used as a standard for comparison purposes.

Compounds 15, 26, 27, 28, 30 and 32 were incorporated onto individual guide strands.

Compound 28 was incorporated onto the 5′ end of the passenger during solid phase synthesis as a dinucleotide.

All the siRNA passenger strands contained a C6-NH₂ conjugation handle on the 5′ end.

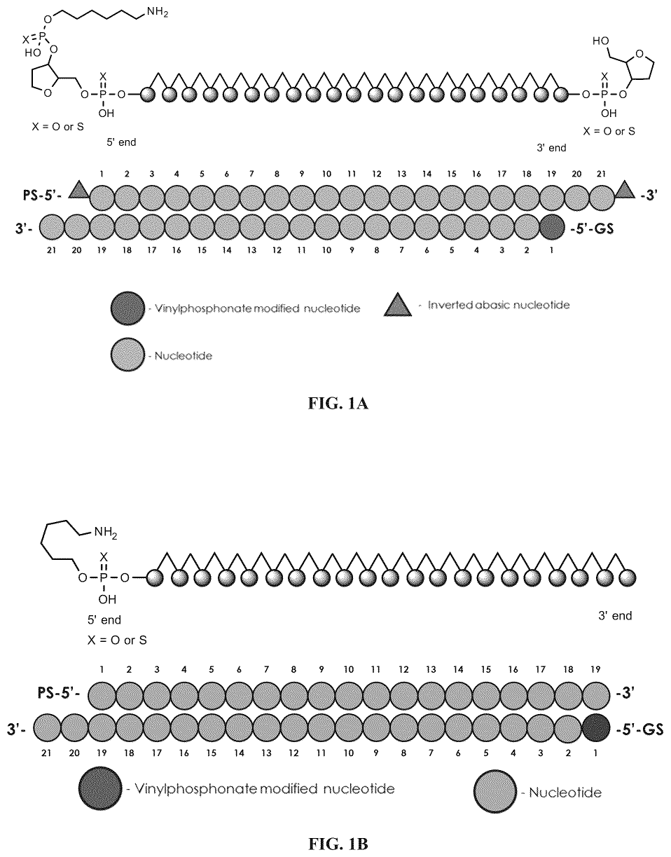

For the 21mer duplex with 19 bases of complementarity and 3′ dinucleotide overhangs, the conjugation handle was connected to siRNA passenger strand via an inverted abasic phosphodiester, (see FIG. 1A).

For the blunt ended duplex with 19 bases of complementarity and one 3′ dinucleotide overhang the conjugation handle was connected to siRNA passenger strand via a phosphodiester on the terminal base, (see FIG. 1B).

Purified single strands were duplexed to obtain double stranded siRNA.

### Example 11. In Vitro Activity of Vinylphosphonate Modified Nucleotide Structures in HCT116 Cells

siRNA design and synthesis: A 21mer HPRT guide strand was designed against mouse HPRT. The sequence (5′ to 3′) of the guide/antisense strand was UUAAAAUCUACAGUCAUAGUU (SEQ ID NO: 1275).

Three versions were prepared incorporating vinylphosphonate modified nucleotide structures (compounds 3, 15 and 27). The guide and fully complementary 0RNA passenger strands were assembled on solid phase using standard phospharamidite chemistry, and purified over HPLC. Base, sugar and phosphate modifications that are well described in the field of RNAi were used to optimize the potency of the duplex and reduce immunogenicity. Purified single strands were duplexed to obtain double stranded siRNA as described in FIG. 1B.

In vitro study: The different siRNAs were tranfected into human colorectal carcinoma HCT116 cells at 100 nM, 10 nM, 1 nM, 0.1 nM, 0.01 nM, 0.001 nM, and 0.0001 nM final concentration. The siRNAs were formulated with commercially available transfection reagent, Lipofectamine RNAiMAX (Life Technologies), according to the manufacturer's “forward transfection” instructions. Cells were plated 24 h prior to transfection in triplicate on 24-well tissue culture plates, with 50000 cells per well. At 48 h post-transfection cells were washed with PBS and harvested with TRIzol® reagent (Life Technologies). RNA was isolated using the Direct-zol-96 RNA Kit (Zymo Research) according to the manufacturer's instructions. 10 μl of RNA was reverse transcribed to cDNA using the High Capacity cDNA Reverse Transcription Kit (Applied Biosystems) according to the manufacturer's instructions. cDNA samples were evaluated by qPCR with HPRT-specific and PPIB-specific TaqMan gene expression probes (Thermo Fisher) using TaqMan® Fast Advanced Master Mix (Applied Biosystems). HPRT values were normalized within each sample to PPIB gene expression. The quantification of HPRT downregulation was performed using the standard 2^{-ΔΔct} method. All experiments were performed in triplicate.

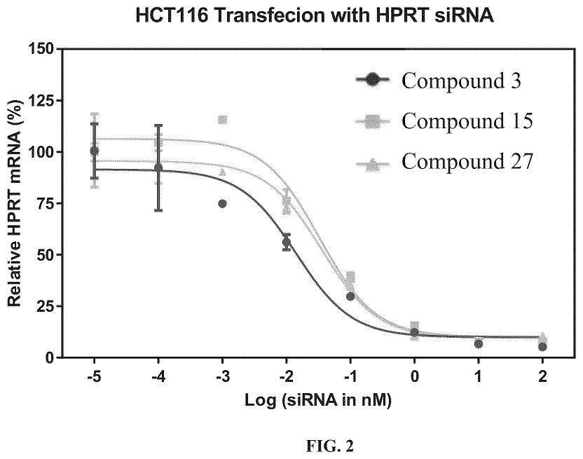

FIG. 2 shows the dose response curves demonstrating that novel vinylphosphonate modified nucleotide structures on the guide strands of an HPRT siRNA, after in vitro transfection of the duplex, can be loaded into RISC and mediate sequence specific down regulation of the target HPRT gene. Activity of the analogues (compounds 15 and 27) was comparable to the standard vinylphosphonate modified nucleotide (compound 3).

### Example 12. In Vitro Activity of Vinylphosphonate Modified Nucleotide Structures in Rhabdomyosarcoma Cells

siRNA design and synthesis: A 21mer myostatin (MSTN) guide strand was designed against human MSTN. The sequence (5′ to 3′) of the guide/antisense strand was UUAUUAUUUGUUCUUUGCCUU (SEQ ID NO: 1273). Five versions were prepared incorporating different structures (compounds 3, 15, 26, 27 and 28). The guide and fully complementary RNA passenger strands were assembled on solid phase using standard phospharamidite chemistry, and purified over HPLC. Base, sugar and phosphate modifications that are well described in the field of RNAi were used to optimize the potency of the duplex and reduce immunogenicity. Purified single strands were duplexed to obtain double stranded siRNA as described in FIG. 1A.

In vitro study: The activity of the siRNAs was evaluated in transfected human rhabdomyosarcoma cells (SJCRH30, ATCC CRL-2061). Cells were grown in RPMI-1640 supplemented with 10% heat inactivated FBS (Gibco) and 10 mM HEPES and 1 mM sodium pyruvate. For siRNA transfections, cells were plated at a density of 20.000 cells/well on 24 well plates plates and transfected with various concentrations of the siRNAs (0.0001-100 nM final concentration) using Lipofectamine RNAiMAX (Life Technologies), according to the manufacturer's “forward transfection” instructions. At 72 h post-transfection cells were washed with PBS and harvested with 300 l/well TRIzol® reagent (Life Technologies). RNA was isolated using the Direct-zol-96 RNA Kit (Zymo Research) according to the manufacturer's instructions. 10 μl of RNA was reverse transcribed to cDNA using the High Capacity cDNA Reverse Transcription Kit (Applied Biosystems) according to the manufacturer's instructions. cDNA samples were evaluated by qPCR with MSTN-specific and PPIB-specific TaqMan gene expression probes (Thermo Fisher) using TaqMan® Fast Advanced Master Mix (Applied Biosystems) and the ΔΔCt method. Individual experiments were performed in duplicate or triplicate.

The table below shows the the half maximal inhibitory concentrations of the analogs (compounds 15, 26, 27 and 28) and maximium knockdown achieved relative to the standard vinylphosphonate modified nucleotide (compound 3).

| Compound on 5′ end of GS | 3 | 28 | 26 | 15 | 27 |
|---|---|---|---|---|---|
| IC50 (pM) | 74.0 | 55.7 | 86.5 | 80.3 | 16.2 |
| Max KD | 87.77 | 86.51 | 84.61 | 85.47 | 87.24 |
| (% untreated control) | | | | | |

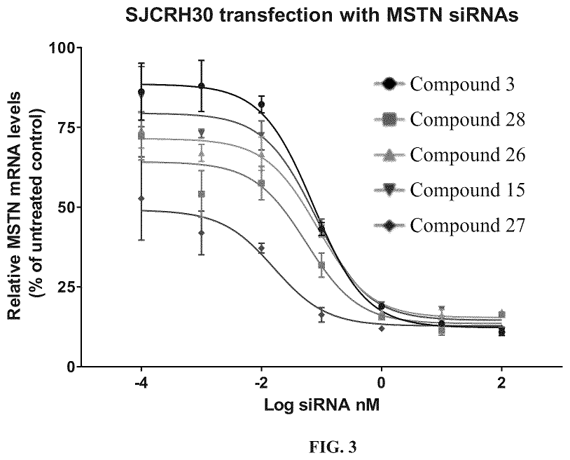

FIG. 3 shows the dose response curves, demonstrating that novel vinylphosphonate modified nucleotide structures on the guide strands of an MSTN siRNA, after in vitro transfection of the duplex, can be loaded into RISC and mediate sequence specific down regulation of the target MSTN gene. Activity of the analogs (compounds 15, 26, 27 and 28) was comparable to the standard vinylphosphonate modified nucleotide (compound 3).

### Example 13. 2017—PK-407-WT: In Vivo Transferrin mAb Conjugate Delivery of siRNA

For groups 1-4, the 21mer HPRT guide strand was designed against mouse HPRT. The sequence (5′ to 3′) of the guide/antisense strand was UUAAAAUCUACAGUCAUAGUU (SEQ ID NO: 1275). The guide and fully complementary RNA passenger strands were assembled on solid phase using standard phospharamidite chemistry and purified over HPLC. Base, sugar and phosphate modifications that are well described in the field of RNAi were used to optimize the potency of the duplex and reduce immunogenicity. Purified single strands were duplexed to obtain double stranded siRNA described above. The guide strand utilized the compound 3 vinylphosphonate modified nucleotide structure. The passenger strand had a conjugation handle on the 5′ end via an inverted abasic phosphodiester linkage, see FIG. 1A.

For groups 5-8, the 21mer HPRT guide strand was designed against mouse HPRT. The sequence (5′ to 3′) of the guide/antisense strand was UUAAAAUCUACAGUCAUAGUU (SEQ ID NO: 1275). The guide and fully complementary RNA passenger strands were assembled on solid phase using standard phospharamidite chemistry and purified over HPLC. Base, sugar and phosphate modifications that are well described in the field of RNAi were used to optimize the potency of the duplex and reduce immunogenicity. Purified single strands were duplexed to obtain double stranded siRNA described in figure C. The guide strand utilized the compound 15 vinylphosphonate modified nucleotide structure. The passenger strand had a conjugation handle on the 5′ end via the phosphodiester linkage on the terminal base, see FIG. 1B.

For groups 9-12, the 21mer HPRT guide strand was designed against mouse HPRT. The sequence (5′ to 3′) of the guide/antisense strand was UUAAAAUCUACAGUCAUAGUU (SEQ ID NO: 1275). The guide and fully complementary RNA passenger strands were assembled on solid phase using standard phospharamidite chemistry and purified over HPLC. Base, sugar and phosphate modifications that are well described in the field of RNAi were used to optimize the potency of the duplex and reduce immunogenicity. Purified single strands were duplexed to obtain double stranded siRNA. The guide strand utilized the compound 27 vinylphosphonate modified nucleotide structure. The passenger strand had a conjugation handle on the 5′ end via the phosphodiester linkage on the terminal base, see FIG. 1B

For groups 13-16, the 21mer MSTN guide strand was designed against human MSTN. The sequence (5′ to 3′) of the guide/antisense strand was UUAUUAUUUGUUCUUUGCCUU (SEQ ID NO: 1273). The guide and fully complementary RNA passenger strands were assembled on solid phase using standard phospharamidite chemistry and purified over HPLC. Base, sugar and phosphate modifications that are well described in the field of RNAi were used to optimize the potency of the duplex and reduce immunogenicity. Purified single strands were duplexed to obtain double stranded siRNA. The guide strand utilized the compound 28 vinylphosphonate modified nucleotide structure. The passenger strand had a conjugation handle on the 5′ end via an inverted abasic phosphodiester linkage, see FIG. 1A.

For groups 17-20, the 21mer MSTN guide strand was designed against human MSTN. The sequence (5′ to 3′) of the guide/antisense strand was UUAUUAUUUGUUCUUUGCCUU (SEQ ID NO: 1273). The guide and fully complementary RNA passenger strands were assembled on solid phase using standard phospharamidite chemistry and purified over HPLC. Base, sugar and phosphate modifications that are well described in the field of RNAi were used to optimize the potency of the duplex and reduce immunogenicity. Purified single strands were duplexed to obtain double stranded siRNA. The guide strand utilized the compound 3 vinylphosphonate modified nucleotide structure. The passenger strand had a conjugation handle on the 5′ end via an inverted abasic phosphodiester linkage, see FIG. 1A

Antibody siRNA Conjugate Synthesis Using Bis-Maleimide (BisMal) Linker
Step 1: Antibody Reduction with TCEP

Antibody was buffer exchanged with 25 mM borate buffer (pH 8) with 1 mM DTPA and made up to 10 mg/ml concentration. To this solution, 4 equivalents of TCEP in the same borate buffer were added and incubated for 2 hours at 37° C. The resultant reaction mixture was combined with a solution of BisMal-siRNA (1.25 equivalents) in pH 6.0 10 mM acetate buffer at RT and kept at 4° C. overnight. Analysis of the reaction mixture by analytical SAX column chromatography showed antibody siRNA conjugate along with unreacted antibody and siRNA. The reaction mixture was treated with 10 EQ of N-ethylmaleimide (in DMSO at 10 mg/mL) to cap any remaining free cysteine residues.

### Step 2: Purification

The crude reaction mixture was purified by AKTA Pure FPLC using anion exchange chromatography (SAX) method-1. Fractions containing DAR1 and DAR2 antibody-siRNA conjugates were isolated, concentrated and buffer exchanged with pH 7.4 PBS.

### Anion Exchange Chromatography (SAX) Method-1

1. Column: Tosoh Bioscience, TSKGel SuperQ-5PW, 21.5 mm ID×15 cm, 13 μm
2. Solvent A: 20 mM TRIS buffer, pH 8.0; Solvent B: 20 mM TRIS, 1.5 M NaCl, pH 8.0; Flow Rate: 6.0 ml/min
3. Gradient:

| a. | % A | % B | Column Volume |
|---|---|---|---|
| b. | 100 | 0 | 1 |
| c. | 81 | 19 | 0.5 |
| d. | 50 | 50 | 13 |
| e. | 40 | 60 | 0.5 |
| f | 0 | 100 | 0.5 |
| g. | 100 | 0 | 2 |

### Anion Exchange Chromatography (SAX) Method-2

1. Column: Thermo Scientific, ProPac™ SAX-10, Bio LC™, 4×250 mm
2. Solvent A: 80% 10 mM TRIS pH 8, 20% ethanol; Solvent B: 80% 10 mM TRIS pH 8, 20% ethanol, 1.5 M NaCl; Flow Rate: 0.75 ml/min
3. Gradient:

| a. | Time | % A | % B |
|---|---|---|---|
| b. | 0.0 | 90 | 10 |
| c. | 3.00 | 90 | 10 |
| d. | 11.00 | 40 | 60 |
| e. | 14.00 | 40 | 60 |
| f. | 15.00 | 20 | 80 |
| g. | 16.00 | 90 | 10 |
| h. | 20.00 | 90 | 10 |

### Step-3: Analysis of the Purified Conjugate

The purity of the conjugate was assessed by analytical HPLC using anion exchange chromatography method-2.

| | SAX | |
|---|---|---|
| | retention | % purity |
| | time | (by peak |
| Conjugate | (min) | area) |
| TfR-mAb-compound 3-HPRT DAR 1 | 9.41 | 99 |
| TfR-mAb-compound 15-HPRT DAR 1 | 8.83 | 99 |
| TfR-mAb-compound 27-HPRT DAR 1 | 8.54 | 99 |
| TfR-mAb-compound 28-MSTN DAR 1 | 8.96 | 99 |
| TfR-mAb-compound 3-MSTN DAR 1 | 9.39 | 99 |

### In Vivo Study

The conjugates were assessed for their ability to mediate mRNA downregulation of myostatin (MSTN) and HPRT in skeletal muscle, in an in vivo experiment (wild type CD-1 mice). Mice were dosed via intravenous (iv) injection with PBS vehicle control and the indicated ASCs and doses, see table below. After 168 hours, gastrocnemius (gastroc) muscle tissues were harvested and snap-frozen in liquid nitrogen. mRNA knockdown in target tissue was determined using a comparative PCR assay. Total RNA was extracted from the tissue, reverse transcribed and mRNA levels were quantified using TaqMan qPCR, using the appropriately designed primers and probes. PPIB (housekeeping gene) was used as an internal RNA loading control, results were calculated by the comparative Ct method, where the difference between the target gene Ct value and the PPIB Ct value (ΔCt) is calculated and then further normalized relative to the PBS control group by taking a second difference (ΔΔCt).

| | | | siRNA | Dose | Harvest |
|---|---|---|---|---|---|
| | | | Dose | Volume | Time |
| Group | Test Article | N | (mg/kg) | (mL/kg) | (h) |
| 1 | TfR1-mAb-compound 3- | 4 | 3 | 5.0 | 168 |
| | HPRT DAR1 | | | | |
| 2 | TfR1-mAb-compound 3- | 4 | 1 | 5.0 | 168 |
| | HPRT DAR1 | | | | |
| 3 | TfR1-mAb-compound 3- | 4 | 0.3 | 5.0 | 168 |
| | HPRT DAR1 | | | | |
| 4 | TfR1-mAb-compound 3- | 4 | 0.1 | 5.0 | 168 |
| | HPRT DAR1 | | | | |
| 5 | TfR1-mAb-compound 15- | 4 | 3.0 | 5.0 | 168 |
| | HPRT DAR1 | | | | |
| 6 | TfR1-mAb-compound 15- | 4 | 1 | 5.0 | 168 |
| | HPRT DAR1 | | | | |
| 7 | TfR1-mAb-compound 15- | 4 | 0.3 | 5.0 | 168 |
| | HPRT DAR1 | | | | |
| 8 | TfR1-mAb-compound 15- | 4 | 0.1 | 5.0 | 168 |
| | HPRT DAR1 | | | | |
| 9 | TfR1-mAb-compound 27- | 4 | 0.5 | 5.0 | 168 |
| | HPRT DAR1 | | | | |
| 10 | TfR1-mAb-compound 27- | 4 | 0.5 | 5.0 | 168 |
| | HPRT DAR1 | | | | |
| 11 | TfR1-mAb-compound 27- | 4 | 0.5 | 5.0 | 168 |
| | HPRT DAR1 | | | | |
| 12 | TfR1-mAb-compound 27- | 4 | 0.5 | 5.0 | 168 |
| | HPRT DAR1 | | | | |
| 13 | TfR1-mAb-compound 28- | 4 | 0.5 | 5.0 | 168 |
| | MSTN DAR1 | | | | |
| 14 | TfR1-mAb-compound 28- | 4 | 0.5 | 5.0 | 168 |
| | MSTN DAR1 | | | | |
| 15 | TfR1-mAb-compound 28- | 4 | 0.5 | 5.0 | 168 |
| | MSTN DAR1 | | | | |
| 16 | TfR1-mAb-compound 28- | 4 | 0.5 | 5.0 | 168 |
| | MSTN DAR1 | | | | |
| 17 | TfR1-mAb-compound 3- | 4 | 0.5 | 5.0 | 168 |
| | MSTN DAR1 | | | | |
| 18 | TfR1-mAb-compound 3- | 4 | 0.5 | 5.0 | 168 |
| | MSTN DAR1 | | | | |
| 19 | TfR1-mAb-compound 3- | 4 | 0.5 | 5.0 | 168 |
| | MSTN DAR1 | | | | |
| 20 | TfR1-mAb-compound 3- | 4 | 0.5 | 5.0 | 168 |
| | MSTN DAR1 | | | | |
| 21 | PBS control | 5 | | 5.0 | 168 |

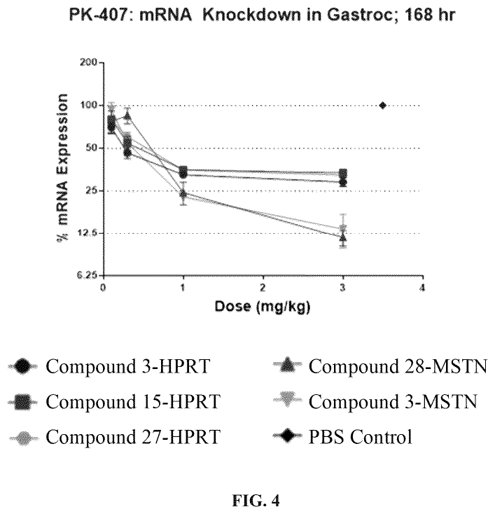

FIG. 4 shows that the vinylphosphonate modified nucleotide structures on the siRNA guide strands demonstrated dose dependent downregulation of their target gene in gastric muscle when conjugated to an anti-TfR mAb targeting the transferrin receptor. Activity of the analogues was comparable to the standard vinylphosphonate modified nucleotide structure.

### Example 14. 2017—PK-421-WT: In Vivo Transferrin mAb Conjugate Delivery of siRNA

For groups 1-12, the 21mer MSTN guide strand was designed against human/mouse MSTN. The sequence (5′ to 3′) of the guide/antisense strand was UUAUUAUUUGUUCUUUGCCUU (SEQ ID NO: 1273). The guide and fully complementary RNA passenger strands were assembled on solid phase using standard phospharamidite chemistry and purified over HPLC. Base, sugar and phosphate modifications that are well described in the field of RNAi were used to optimize the potency of the duplex and reduce immunogenicity. Purified single strands were duplexed to obtain double stranded siRNA. Three guide strands were produced with three different vinylphosphonate modified nucleotide structures at the 5′end (compounds 3, 15 and 26). The passenger strand had a conjugation handle on the 5′ end via a phosphodiester-inverted abasic linkage.

The anti-TfR1 mAb-MSTN DAR1 conjugates were synthesized as described in FIG. 16.

Step 1: Antibody Reduction with TCEP

Antibody was buffer exchanged with 25 mM borate buffer (pH 8) with 1 mM DTPA and made up to 10 mg/ml concentration. To this solution, 4 equivalents of TCEP in the same borate buffer were added and incubated for 2 hours at 37° C. The resultant reaction mixture was combined with a solution of BisMal-siRNA (1.25 equivalents) in 10 mM acetate buffer (pH 6) at RT and kept at 4° C. overnight. Analysis of the reaction mixture by analytical SAX column chromatography showed antibody siRNA conjugate along with unreacted antibody and siRNA. The reaction mixture was treated with 10 EQ of N-ethylmaleimide (in DMSO at 10 mg/mL) to cap any remaining free cysteine residues.

### Step 2: Purification

The crude reaction mixture was purified by AKTA Pure FPLC using anion exchange chromatography (SAX) method-1. Fractions containing DAR1 and DAR2 antibody-siRNA conjugates were isolated, concentrated and buffer exchanged with pH 7.4 PBS.

### Anion Exchange Chromatography (SAX) Method-1.

1. Column: Tosoh Bioscience, TSKGel SuperQ-5PW, 21.5 mm ID×15 cm, 13 μm
2. Solvent A: 20 mM TRIS buffer, pH 8.0; Solvent B: 20 mM TRIS, 1.5 M NaCl, pH 8.0; Flow Rate: 6.0 ml/min
3. Gradient:

| a. | % A | % B | Column Volume |
|---|---|---|---|
| b. | 100 | 0 | 1 |
| c. | 81 | 19 | 0.5 |
| d. | 50 | 50 | 13 |
| e. | 40 | 60 | 0.5 |
| f | 0 | 100 | 0.5 |
| g. | 100 | 0 | 2 |

### Anion Exchange Chromatography (SAX) Method-2

1. Column: Thermo Scientific, ProPac™ SAX-10, Bio LC™, 4×250 mm
2. Solvent A: 80% 10 mM TRIS pH 8, 20% ethanol; Solvent B: 80% 10 mM TRIS pH 8, 20% ethanol, 1.5 M NaCl; Flow Rate: 0.75 ml/min
3. Gradient:

| a. | Time | % A | % B |
|---|---|---|---|
| b. | 0.0 | 90 | 10 |
| c. | 3.00 | 90 | 10 |
| d. | 11.00 | 40 | 60 |
| e. | 14.00 | 40 | 60 |
| f. | 15.00 | 20 | 80 |
| g. | 16.00 | 90 | 10 |
| h. | 20.00 | 90 | 10 |

### Step-3: Analysis of the Purified Conjugate

The purity of the conjugate was assessed by analytical HPLC using anion exchange chromatography method-2.

| | SAX | |
|---|---|---|
| | retention | % purity |
| | time | (by peak |
| Conjugate | (min) | area) |
| TfR-compound 3-MSTN.mff3s DAR 1 | 8.99 | 99 |
| TfR-compound 15-MSTN DAR 1 | 9.12 | 99 |
| TfR-compound 26-MSTN DAR 1 | 9.09 | 99 |

### In Vivo Study

The conjugates were assessed for their ability to mediate mRNA downregulation of myostatin (MSTN) in skeletal muscle, in an in vivo experiment (wild type CD-1 mice). Mice were dosed via intravenous (iv) injection with PBS vehicle control and the indicated ASCs and doses, as noted in the table below. After 168 hours, gastrocnemius (gastroc), quadriceps (quad) and heart muscle tissues were harvested and snap-frozen in liquid nitrogen. mRNA knockdown in target tissue was determined using a comparative qPCR assay as described in the methods section. Total RNA was extracted from the tissue, reverse transcribed and mRNA levels were quantified using TaqMan qPCR, using the appropriately designed primers and probes. PPIB (housekeeping gene) was used as an internal RNA loading control, results were calculated by the comparative Ct method, where the difference between the target gene Ct value and the PPIB Ct value (ΔCt) is calculated and then further normalized relative to the PBS control group by taking a second difference (ΔΔCt).

| | | | siRNA | Dose | Harvest |
|---|---|---|---|---|---|
| | | | Dose | Volume | Time |
| Group | Test Article | N | (mg/kg) | (mL/kg) | (h) |
| 1 | TfR1-mAb-compound 26- | 4 | 3 | 5.0 | 168 |
| | MSTN DAR1 | | | | |
| 2 | TfR1-mAb-compound 26- | 4 | 1 | 5.0 | 168 |
| | MSTN DAR1 | | | | |
| 3 | TfR1-mAb-compound 26- | 4 | 0.3 | 5.0 | 168 |
| | MSTN DAR1 | | | | |
| 4 | TfR1-mAb-compound 26- | 4 | 0.1 | 5.0 | 168 |
| | MSTN DAR1 | | | | |
| 5 | TfR1-mAb-compound 15- | 4 | 3.0 | 5.0 | 168 |
| | MSTN DAR1 | | | | |
| 6 | TfR1-mAb-compound 15- | 4 | 1 | 5.0 | 168 |
| | MSTN DAR1 | | | | |
| 7 | TfR1-mAb-compound 15- | 4 | 0.3 | 5.0 | 168 |
| | MSTN DAR1 | | | | |
| 8 | TfR1-mAb-compound 15- | 4 | 0.1 | 5.0 | 168 |
| | MSTN DAR1 | | | | |
| 9 | TfR1-mAb-compound 3- | 4 | 0.5 | 5.0 | 168 |
| | MSTN DAR1 | | | | |
| 10 | TfR1-mAb-compound 3- | 4 | 0.5 | 5.0 | 168 |
| | MSTN DAR1 | | | | |
| 11 | TfR1-mAb-compound 3- | 4 | 0.5 | 5.0 | 168 |
| | MSTN DAR1 | | | | |
| 12 | TfR1-mAb-compound 3- | 4 | 0.5 | 5.0 | 168 |
| | MSTN DAR1 | | | | |
| 13 | PBS control | 5 | | 5.0 | 168 |

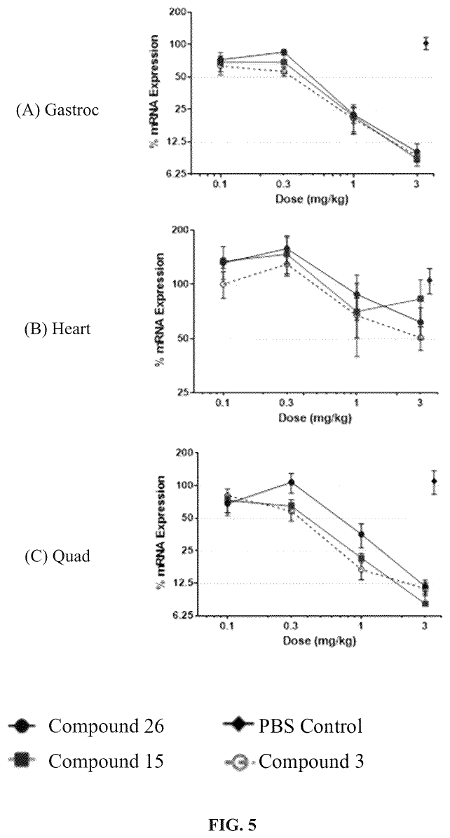

FIG. 5 demonstrates MSTN mRNA downregulation in gastroc, quad and heart muscle with antibody siRNA conjugates containing different vinylphosphonate modified nucleotide structure on the 5′ end of the guide strand. Activity of the analogues was comparable to the standard vinylphosphonate modified nucleotide structure.

### Example 15. 2017—PK-422-WT: In Vivo Transferrin mAb Conjugate Delivery of siRNA

For groups 1-4 and 9-12, the 21mer HPRT guide strand was designed against mouse HPRT. The sequence (5′ to 3′) of the guide/antisense strand was UUAAAAUCUACAGUCAUAGUU (SEQ ID NO: 1275). The guide and fully complementary RNA passenger strands were assembled on solid phase using standard phospharamidite chemistry and purified over HPLC. Base, sugar and phosphate modifications that are well described in the field of RNAi were used to optimize the potency of the duplex and reduce immunogenicity. Purified single strands were duplexed to obtain double stranded siRNA. The guide strand was produced with compound 3 vinylphosphonate modified nucleotide structures at the 5′end. The passenger strand had a conjugation handle on the 5′ end via aphosphorothioate-inverted abasic-phosphodiester linker, see FIG. 1A.

For groups 5-8 and 13-16 a 21mer HPRT guide strand was designed against mouse HPRT. The sequence (5′ to 3′) of the guide/antisense strand was UUAAAAUCUACAGUCAUAGUU (SEQ ID NO: 1275). The guide and fully complementary RNA passenger strands were assembled on solid phase using standard phospharamidite chemistry and purified over HPLC. Base, sugar and phosphate modifications that are well described in the field of RNAi were used to optimize the potency of the duplex and reduce immunogenicity. Purified single strands were duplexed to obtain double stranded siRNA. The guide strand was produced with a compound 26 vinylphosphonate modified nucleotide structures at the 5′end. The passenger strand had a conjugation handle on the 5′ end via the terminal phosphorothioate, see FIG. 1B.

The anti-TfR1 mAb-HPRT DAR1 conjugates were synthesized as described in FIG. 16.

Step 1: Antibody Reduction with TCEP

Antibody was buffer exchanged with 25 mM borate buffer (pH 8) with 1 mM DTPA and made up to 10 mg/ml concentration. To this solution, 4 equivalents of TCEP in the same borate buffer were added and incubated for 2 hours at 37° C. The resultant reaction mixture was combined with a solution of BisMal-siRNA (1.25 equivalents) in 10 mM acetate buffer (pH 6) at RT and kept at 4° C. overnight. Analysis of the reaction mixture by analytical SAX column chromatography showed antibody siRNA conjugate along with unreacted antibody and siRNA. The reaction mixture was treated with 10 EQ of N-ethylmaleimide (in DMSO at 10 mg/mL) to cap any remaining free cysteine residues.

### Step 2: Purification

The crude reaction mixture was purified by AKTA Pure FPLC using anion exchange chromatography (SAX) method-1. Fractions containing DAR1 and DAR2 antibody-siRNA conjugates were isolated, concentrated and buffer exchanged with pH 7.4 PBS.

### Anion Exchange Chromatography (SAX) Method-1.

1. Column: Tosoh Bioscience, TSKGel SuperQ-5PW, 21.5 mm ID×15 cm, 13 μm
2. Solvent A: 20 mM TRIS buffer, pH 8.0; Solvent B: 20 mM TRIS, 1.5 M NaCl, pH 8.0; Flow Rate: 6.0 ml/min
3. Gradient:

| a. | % A | % B | Column Volume |
|---|---|---|---|
| b. | 100 | 0 | 1 |
| c. | 81 | 19 | 0.5 |
| d. | 50 | 50 | 13 |
| e. | 40 | 60 | 0.5 |
| f | 0 | 100 | 0.5 |
| g. | 100 | 0 | 2 |

### Anion Exchange Chromatography (SAX) Method-2

1. Column: Thermo Scientific, ProPac™ SAX-10, Bio LC™, 4×250 mm
2. Solvent A: 80% 10 mM TRIS pH 8, 20% ethanol; Solvent B: 80% 10 mM TRIS pH 8, 20% ethanol, 1.5 M NaCl; Flow Rate: 0.75 ml/min
3. Gradient:

| a. | Time | % A | % B |
|---|---|---|---|
| b. | 0.0 | 90 | 10 |
| c. | 3.00 | 90 | 10 |
| d. | 11.00 | 40 | 60 |
| e. | 14.00 | 40 | 60 |
| f | 15.00 | 20 | 80 |
| g. | 16.00 | 90 | 10 |
| h. | 20.00 | 90 | 10 |

### Step-3: Analysis of the Purified Conjugate

The purity of the conjugate was assessed by analytical HPLC using anion exchange chromatography method-2.

| | SAX | |
|---|---|---|
| | retention | % purity |
| | time | (by peak |
| Conjugate | (min) | area) |
| TfR-compound 3-HPRT DAR 1 | 9.14 | 99 |
| TfR-compound 26-HPRT DAR 1 | 8.54 | 99 |
| ASGR-compound 3-HPRT DAR 1 | 9.3 | 99 |
| ASGR-compound 26-MSTN DAR 1 | 8.62 | 99 |

### In Vivo Study

The conjugates were assessed for their ability to mediate mRNA downregulation of HPRT in muscle and liver, in an in vivo experiment (wild type CD-1 mice). Mice were dosed via intravenous (iv) injection with PBS vehicle control and the indicated ASCs and doses, as noted in the table below. After 168 hours, gastrocnemius (gastroc), and liver tissues were harvested and snap-frozen in liquid nitrogen. mRNA knockdown in target tissue was determined using a comparative qPCR assay as described in the methods section. Total RNA was extracted from the tissue, reverse transcribed and mRNA levels were quantified using TaqMan qPCR, using the appropriately designed primers and probes. PPIB (housekeeping gene) was used as an internal RNA loading control, results were calculated by the comparative Ct method, where the difference between the target gene Ct value and the PPIB Ct value (ΔCt) is calculated and then further normalized relative to the PBS control group by taking a second difference (ΔΔCt).

| | | | siRNA | Dose | Harvest |
|---|---|---|---|---|---|
| | | | Dose | Volume | Time |
| Group | Test Article | N | (mg/kg) | (mL/kg) | (h) |
| 1 | TfR1-mAb-compound 3- | 4 | 1 | 5.0 | 168 |
| | HPRT DAR1 | | | | |
| 2 | TfR1-mAb-compound 3- | 4 | 0.3 | 5.0 | 168 |
| | HPRT DAR1 | | | | |
| 3 | TfR1-mAb-compound 3- | 4 | 0.1 | 5.0 | 168 |
| | HPRT DAR1 | | | | |
| 4 | TfR1-mAb-compound 3- | 4 | 0.03 | 5.0 | 168 |
| | HPRT DAR1 | | | | |
| 5 | TfR1-mAb-compound 26- | 4 | 1 | 5.0 | 168 |
| | HPRT DAR1 | | | | |
| 6 | TfR1-mAb-compound 26- | 4 | 0.3 | 5.0 | 168 |
| | HPRT DAR1 | | | | |
| 7 | TfR1-mAb-compound 26- | 4 | 0.1 | 5.0 | 168 |
| | HPRT DAR1 | | | | |
| 8 | TfR1-mAb-compound 26- | 4 | 0.03 | 5.0 | 168 |
| | HPRT DAR1 | | | | |
| 9 | ASGR-mAb-compound 3- | 4 | 1 | 5.0 | 168 |
| | HPRT DAR1 | | | | |
| 10 | ASGR-mAb-compound 3- | 4 | 0.3 | 5.0 | 168 |
| | HPRT DAR1 | | | | |
| 11 | ASGR-mAb-compound 3- | 4 | 0.1 | 5.0 | 168 |
| | HPRT DAR1 | | | | |
| 12 | ASGR-mAb-compound 3- | 4 | 0.03 | 5.0 | 168 |
| | HPRT DAR1 | | | | |
| 13 | ASGR-mAb-compound 26- | 4 | 1 | 5.0 | 168 |
| | HPRT DAR1 | | | | |
| 14 | ASGR-mAb-compound 26- | 4 | 0.3 | 5.0 | 168 |
| | HPRT DAR1 | | | | |
| 15 | ASGR-mAb-compound 26- | 4 | 0.1 | 5.0 | 168 |
| | HPRT DAR1 | | | | |
| 16 | ASGR-mAb-compound 26- | 4 | 0.03 | 5.0 | 168 |
| | HPRT DAR1 | | | | |
| 17 | PBS control | 5 | | 5.0 | 168 |

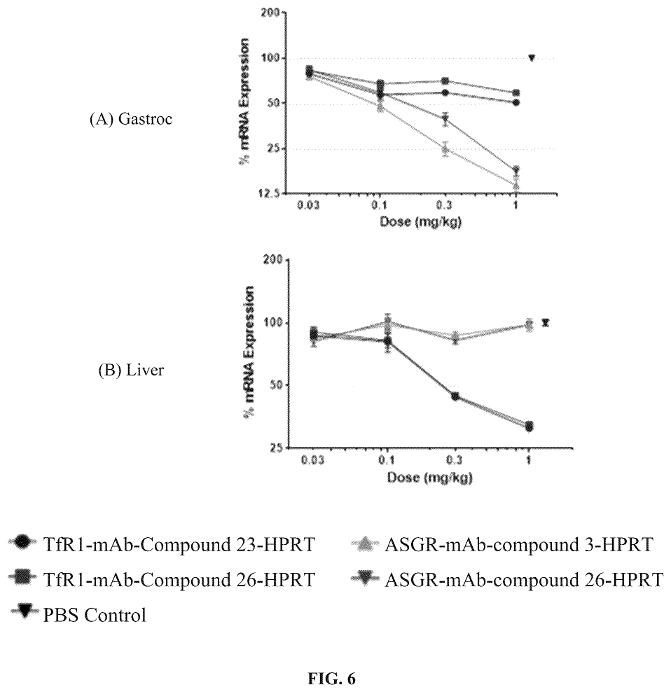

FIG. 6 demonstrates the modified vinylphosphonate modified nucleotide structures on the siRNA guide strands were able to mediate dose dependent downregulation of the HPRT target gene in the mouse liver and gastroc muscle after IV administration to a mouse. When conjugated to an anti-TfR mAb targeting the transferrin receptor on muscle, the siRNA was able to mediate HPRT downregulation in muscle. When conjugated to an anti-ASGR mAb targeting the ASGR receptor on liver hepatocytes, the siRNA was able to mediate HPRT downregulation in liver.

### Example 16. In Vitro Activity of Vinylphosphonate Modified Nucleotide Structures In HCT116 Cells

siRNA design and synthesis: A 21mer SSB guide strand was designed against mouse SSB. The sequence (5′ to 3′) of the guide/antisense strand was UUACAUUAAAGUCUGUUGUUU (SEQ ID NO: 1274). Three versions were made incorporating vinylphosphonate modified nucleotide structures (compounds 3, 30 and 32). The guide and fully complementary RNA passenger strands were assembled on solid phase using standard phospharamidite chemistry, and purified over HPLC. The vinylphosphonate modified nucleotide structures were incorporated using the amidites described in figure A3. The Base, sugar and phosphate modifications that are well described in the field of RNAi were used to optimize the potency of the duplex and reduce immunogenicity. Purified single strands were duplexed to get the double stranded siRNA described above.

In vitro study: The different siRNAs were transfected into human colorectal carcinoma HCT116 cells at 100 nM, 10 nM, 1 nM, 0.1 nM, 0.01 nM, 0.001 nM, and 0.0001 nM final concentration. The siRNAs were formulated with commercially available transfection reagent, Lipofectamine RNAiMAX (Life Technologies), according to the manufacturer's “forward transfection” instructions. Cells were plated 24 h prior to transfection in triplicate on 24-well tissue culture plates, with 50000 cells per well. At 48 h post-transfection cells were washed with PBS and harvested with TRIzol® reagent (Life Technologies). RNA was isolated using the Direct-zol-96 RNA Kit (Zymo Research) according to the manufacturer's instructions. 10 μl of RNA was reverse transcribed to cDNA using the High Capacity cDNA Reverse Transcription Kit (Applied Biosystems) according to the manufacturer's instructions. cDNA samples were evaluated by qPCR with HPRT-specific and PPIB-specific TaqMan gene expression probes (Thermo Fisher) using TaqMan® Fast Advanced Master Mix (Applied Biosystems). HPRT values were normalized within each sample to PPIB gene expression. The quantification of HPRT downregulation was performed using the standard 2^{-ΔΔCt} method. All experiments were performed in triplicate.

EC₅₀ values were as follows:

| Sample | EC50 (pM) |
|---|---|
| Compound 3-SSB | 4.00 |
| Compound 30-SSB | 0.44 |
| Compound 32-SSB | 1.46 |

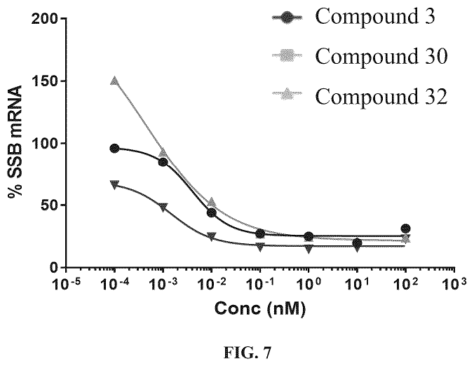

FIG. 7 shows the dose response curves demonstrating that novel vinylphosphonate modified nucleotide structures on the 5′ end of the guide strands of an SSB siRNA, after in vitro transfection of the duplex, can be loaded into RISC and mediate sequence specific down regulation of the target SSB gene. Activity of the analogues (compounds 30 and 32) was comparable to the standard vinylphosphonate modified nucleotide structure (compound 3).

### Example 17. In Vitro Activity Vinylphosphonate Modified Nucleotide Structures in Rhabdomyosarcoma Cells

siRNA design and synthesis: A 21mer myostatin (MSTN) guide strand was designed against mouse and human MSTN. The sequence (5′ to 3′) of the guide/antisense strand was UUAUUAUUUGUUCUUUGCCUU (SEQ ID NO: 1273). Four versions were prepared incorporating different structures (compounds 3, 26, 30 and 32). The guide and fully complementary RNA passenger strands were assembled on solid phase using standard phospharamidite chemistry, and purified using HPLC. The vinylphosphonate modified nucleotide structures were incorporated using the amidites as described above. Base, sugar and phosphate modifications that are well described in the field of RNAi were used to optimize the potency of the duplex and reduce immunogenicity. Purified single strands were duplexed to get the double stranded siRNA as described above.

In addition, a 21mer SSB guide strand was designed against mouse SSB. The sequence (5′ to 3′) of the guide/antisense strand was UUACAUUAAAGUCUGUUGUUU (SEQ ID NO: 1274). Three versions were made with different vinylphosphonate modified nucleotide structures (compounds 3, 30 and 32). The guide and fully complementary RNA passenger strands were assembled on solid phase using standard phospharamidite chemistry, and purified over HPLC. The vinylphosphonate modified nucleotide structures were incorporated using the amidites as described herein. The Base, sugar and phosphate modifications that are well described in the field of RNAi were used to optimize the potency of the duplex and reduce immunogenicity. Purified single strands were duplexed to get the double stranded siRNA as described above.

In vitro study: The activity of the siRNAs was evaluated in transfected human rhabdomyosarcoma cells (SJCRH30, ATCC CRL-2061). Cells were grown in RPMI-1640 supplemented with 10% heat inactivated FBS (Gibco) and 10 mM HEPES and 1 mM sodium pyruvate. For siRNA transfections, cells were plated at a density of 20.000 cells/well on 24 well plates plates and transfected with various concentrations of the siRNAs (0.0001-100 nM final concentration) using Lipofectamine RNAiMAX (Life Technologies), according to the manufacturer's “forward transfection” instructions. At 72 h post-transfection cells were washed with PBS and harvested with 300 ul/well TRIzol® reagent (Life Technologies). RNA was isolated using the Direct-zol-96 RNA Kit (Zymo Research) according to the manufacturer's instructions. 10 μl of RNA was reverse transcribed to cDNA using the High Capacity cDNA Reverse Transcription Kit (Applied Biosystems) according to the manufacturer's instructions. cDNA samples were evaluated by qPCR with MSTN-specific and PPIB-specific TaqMan gene expression probes (Thermo Fisher) using TaqMan® Fast Advanced Master Mix (Applied Biosystems) and the ΔΔCt method. Individual experiments were performed in duplicate or triplicate.

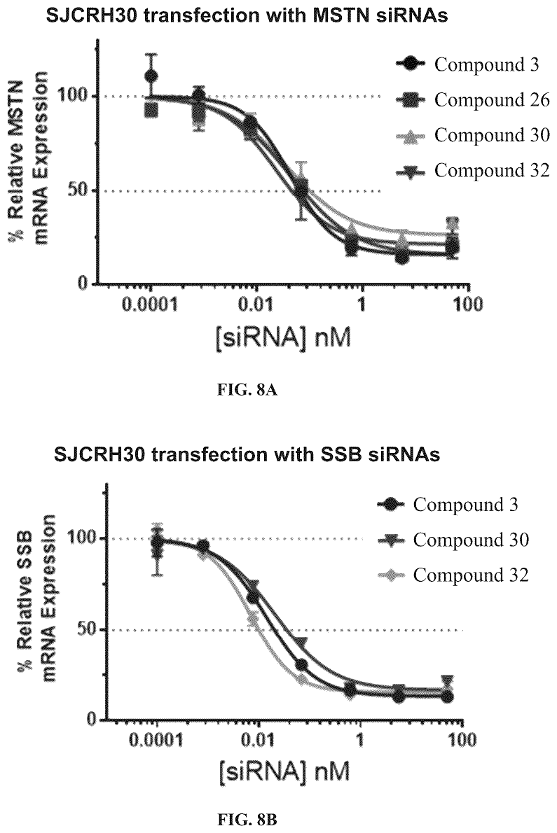

FIG. 8A shows the dose response curves, demonstrating that the novel vinylphosphonate modified nucleotide structures on the guide strands of an MSTN siRNA, after in vitro transfection of the duplex, can be loaded into RISC and mediate sequence specific down regulation of the target MSTN gene. Activity of the analogues (compounds 26, 30 and 32) was comparable to the standard vinylphosphonate modified nucleotide (compound 3).

FIG. 8B shows the dose response curves, demonstrating that the novel vinylphosphonate modified nucleotide structures on the guide strands of an SSB siRNA, after in vitro transfection of the duplex, can be loaded into RISC and mediate sequence specific down regulation of the target SSB gene. Activity of the analogues (30 and 32) was comparable to a standard vinylphosphonate modified nucleotide (compound 3).

### Example 18. In Vitro Activity of Vinyl Phosphonate Modified Nucleotide Structures in Apparently Healthy Human-Derived Immortalized Skeletal Muscle Myoblast Cells (MB)

siRNA design and synthesis: A 21mer myostatin (MSTN) guide strand was designed against mouse and human MSTN. The sequence (5′ to 3′) of the guide/antisense strand was UUAUUAUUUGUUCUUUGCCUU (SEQ ID NO: 1273). Four versions were prepared incorporating different vinylphosphonate modified nucleotide structures (compounds 3, 26, 30 and 32). The guide and fully complementary RNA passenger strands were assembled on solid phase using standard phospharamidite chemistry, and purified using HPLC. The vinylphosphonate modified nucleotide structures were incorporated using the amidites described herein. Base, sugar and phosphate modifications that are well described in the field of RNAi were used to optimize the potency of the duplex and reduce immunogenicity. Purified single strands were duplexed to get the double stranded siRNA as described above.

In addition, a 21mer SSB guide strand was designed against mouse SSB. The sequence (5′ to 3′) of the guide/antisense strand was UUACAUUAAAGUCUGUUGUUU (SEQ ID NO: 1274). Three versions were prepared incorporating different vinylphosphonate modified nucleotide structures (compounds 3, 30 and 32). The guide and fully complementary RNA passenger strands were assembled on solid phase using standard phospharamidite chemistry, and purified over HPLC. The vinylphosphonate modified nucleotide structures were incorporated using the amidites described herein. The Base, sugar and phosphate modifications that are well described in the field of RNAi were used to optimize the potency of the duplex and reduce immunogenicity. Purified single strands were duplexed to get the double stranded siRNA as described above.

In vitro study: The activity of the siRNAs was evaluated in apparently healthy human-derived immortalized skeletal muscle myoblasts (MB) (obtained from Denis Furling, Institut de Myologie, France). MB cells were grown in a complete skeletal muscle cell growth medium (PromoCell). Cells were plated 24 h prior to transfection in triplicate on 96-well tissue culture plates, with 4000 (MB) cells per well. siRNAs were transfected into cells 50 nM, 5.5556 nM, 0.6173 nM, 0.0686 nM, 0.0076 nM, 0.0008 nM, and 0.0001 nM final concentration. The siRNAs were formulated with commercially available transfection reagent Lipofectamine RNAiMAX (Life Technologies), according to the manufacturer's “forward transfection” protocol instructions. At 48 h post-transfection cells were washed with PBS and harvested with TRIzol® reagent (Life Technologies). RNA was isolated using the Direct-zol-96 RNA Kit (Zymo Research) according to the manufacturer's instructions. 10 μl of RNA was reverse transcribed to cDNA using the High Capacity cDNA Reverse Transcription Kit (Applied Biosystems) according to the manufacturer's instructions. cDNA samples were evaluated by qPCR with SSB-specific, MSTN-specific and PPIB-specific TaqMan gene expression probes (Thermo Fisher) using TaqMan® Fast Advanced Master Mix (Applied Biosystems). SSB and MSTN expression values were normalized within each sample to PPIB gene expression. The quantification of SSB and MSTN downregulation was performed using the standard 2-ΔΔCt method. All experiments were performed in triplicate.

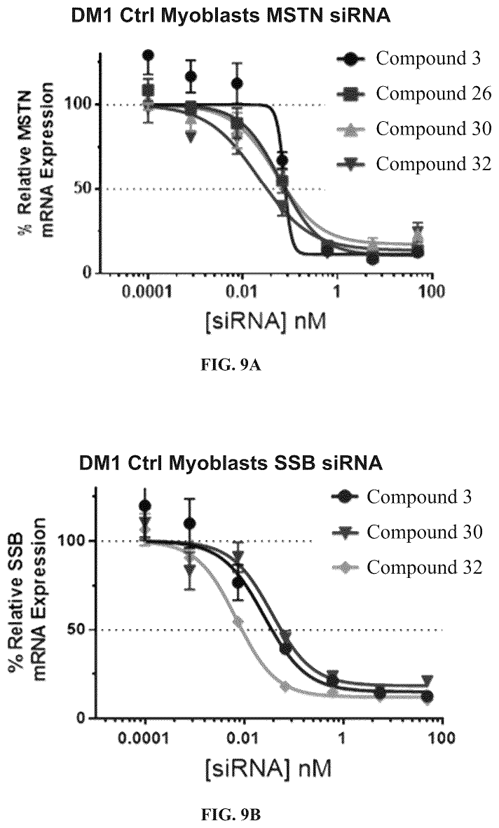

FIG. 9A shows the dose response curves, demonstrating that the novel vinylphosphonate modified nucleotide structures on the guide strands of an MSTN siRNA, after in vitro transfection of the duplex, can be loaded into RISC and mediate sequence specific down regulation of the target MSTN gene. Activity of the analogues (compounds 26, 30 and 32) was comparable to the standard vinylphosphonate modified nucleotide (compound 3).

FIG. 9B shows the dose response curves, demonstrating that the novel vinylphosphonate modified nucleotide structures on the guide strands of an SSB siRNA, after in vitro transfection of the duplex, can be loaded into RISC and mediate sequence specific down regulation of the target SSB gene. Activity of the analogues (30 and 32) was comparable to a standard vinylphosphonate modified nucleotide (compound 3).

While preferred embodiments of the present disclosure have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments of the disclosure described herein may be employed in practicing the disclosure. It is intended that the following claims define the scope of the disclosure and that methods and structures within the scope of these claims and their equivalents be covered thereby.

What is claimed is:

1. A polynucleotide conjugate molecule comprising a binding moiety and a polynucleotide, wherein the polynucleotide comprises (i) at least one 5′-vinylphosphonate modified non-natural nucleotide and (ii) at least one modified internucleotide linkage or at least one inverted abasic moiety; and
wherein the at least one 5′-vinylphosphonate modified non-natural nucleotide is: 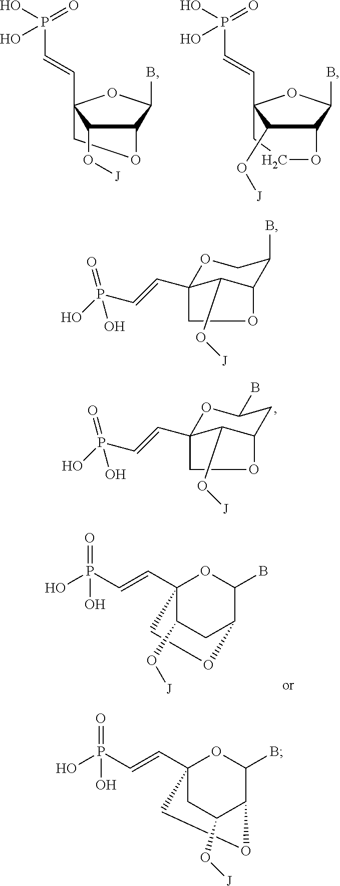
wherein B is a heterocyclic base moiety; and
J is an internucleotide linking group linking to the adjacent nucleotide of the polynucleotide.

2. The molecule of claim 1, wherein the at least one 5′-vinylphosphonate modified non-natural nucleotide is located at the 5′-terminus of the polynucleotide.

3. The molecule of claim 1, wherein the at least one 5′-vinylphosphonate modified non-natural nucleotide is located at an internucleotide linkage of the polynucleotide.

4. The molecule of claim 1, wherein the at least one 5′-vinylphosphonate modified non-natural nucleotide is further modified at the 2′-position.

5. The molecule of claim 5, wherein the 2′-modification is selected from 2′-O-methyl, 2′-O-methoxyethyl (2′-O-MOE), 2′-deoxy, 2′-deoxy-2′-fluoro, 2′-O-aminopropyl (2′-O-AP), 2′-O-dimethylaminoethyl (2′-O-DMAOE), 2′-O-dimethylaminopropyl (2′-O-DMAP), 2′-O-dimethylaminoethyloxyethyl (2′-O-DMAEOE), 2′-O—N-methylacetamido (2′-O-NMA), 2′-O-ethyoxyethyl (2′-O-EOE), 2′-O-(2-N-Methylcarbamoylethyl), PEG1, or PEG2 modified nucleotide.

6. The molecule of claim 1, wherein the at least one modified internucleotide linkage comprises a phosphorothioate linkage, a phosphorodithioate linkage, a phosphorodiamidate linkage, a methylphosphonate linkage, or an amide linkage.

7. The molecule of claim 1, wherein the at least one inverted abasic moiety is at at least one terminus.

8. The molecule of claim 1, wherein the polynucleotide comprises a single-stranded polynucleic acid molecule.

9. The molecule of claim 1, wherein the polynucleotide comprises a first polynucleotide and a second polynucleotide hybridized to the first polynucleotide to form a double-stranded polynucleic acid molecule.

10. The molecule of claim 9, wherein the second polynucleotide comprises at least one modification.

11. The molecule of claim 9, wherein the first polynucleotide and the second polynucleotide are RNA molecules.

12. The molecule of claim 1, wherein the binding moiety and the polynucleotide is coupled via a bond; a C₁-C₆ alkyl group; or a homobifunctional linker or a heterobifunctional linker, optionally conjugated to a C₁-C₆ alkyl group.

13. The molecule of claim 1, further comprising a polymer.

14. The molecule of claim 1, wherein the binding moiety comprises an antibody or antigen binding fragment thereof.

15. The molecule of claim 14, wherein the antibody or antigen binding fragment thereof comprises a humanized antibody or binding fragment thereof, a chimeric antibody or binding fragment thereof, a monoclonal antibody or binding fragment thereof, a monovalent Fab′, a divalent Fab2, a single-chain variable fragment (scFv), a diabody, a minibody, a nanobody, a single-domain antibody (sdAb), or a camelid antibody or binding fragment thereof.

16. The molecule of claim 1, wherein the binding moiety comprises a peptide or a small molecule.

17. The molecule of claim 16, wherein the polymer is polyethylene glycol.

18. The molecule of claim 16, wherein the polymer has a molecular weight of about 1000 Da, 2000 Da, or 5000 Da.

19. The molecule of claim 1, further comprising an endosomolytic moiety.

20. A compound suitable for the synthesis of an oligonucleotide, wherein the compound is selected from the group: 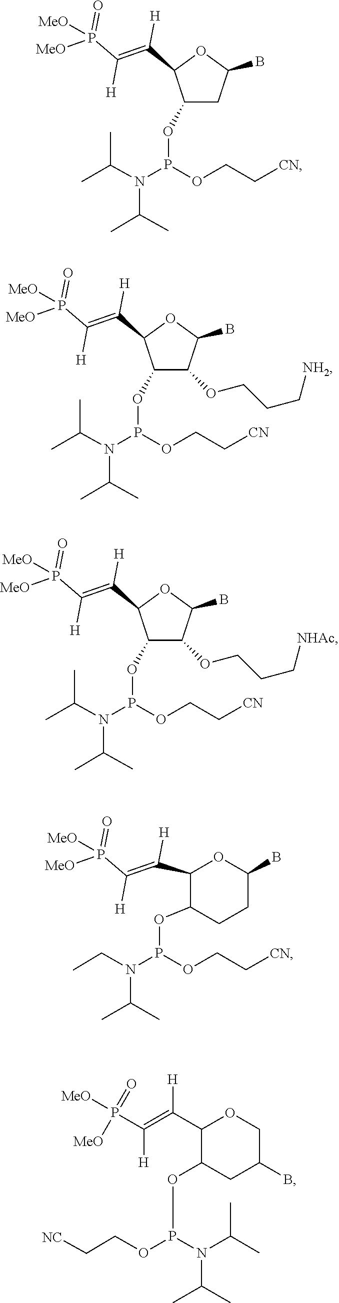 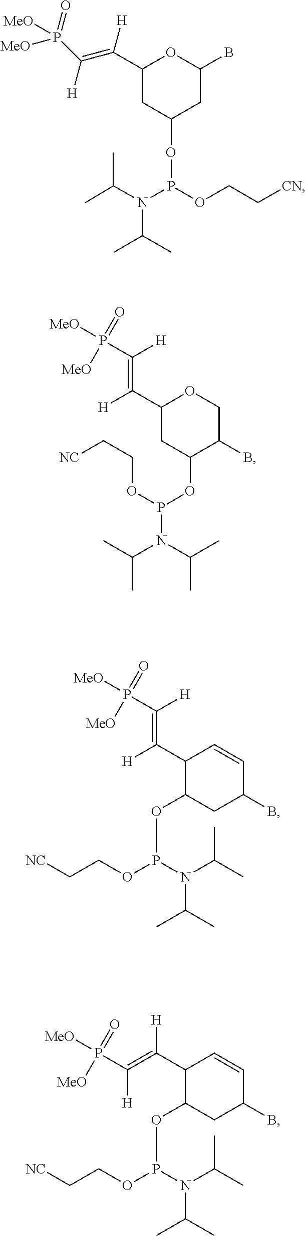 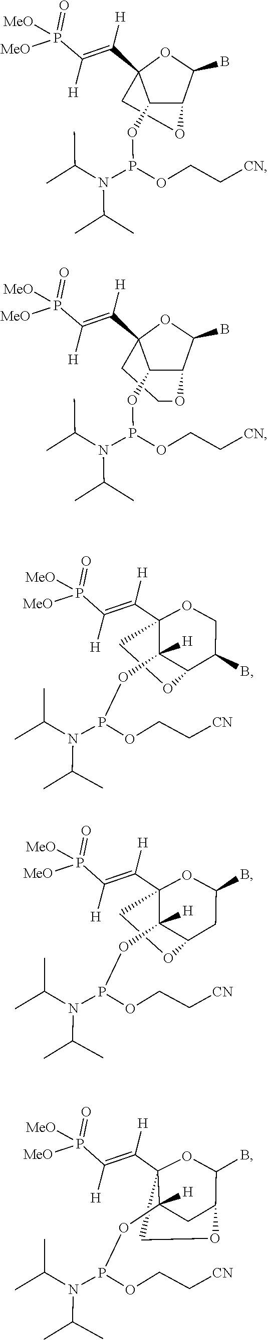 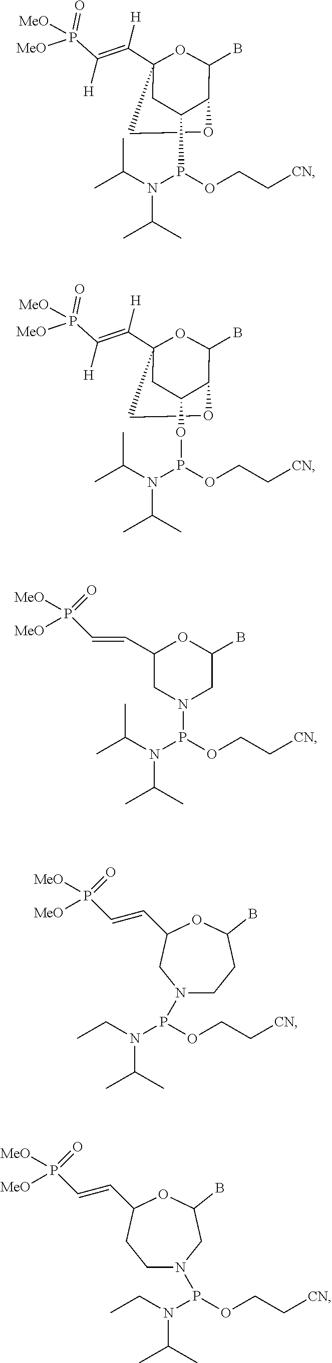 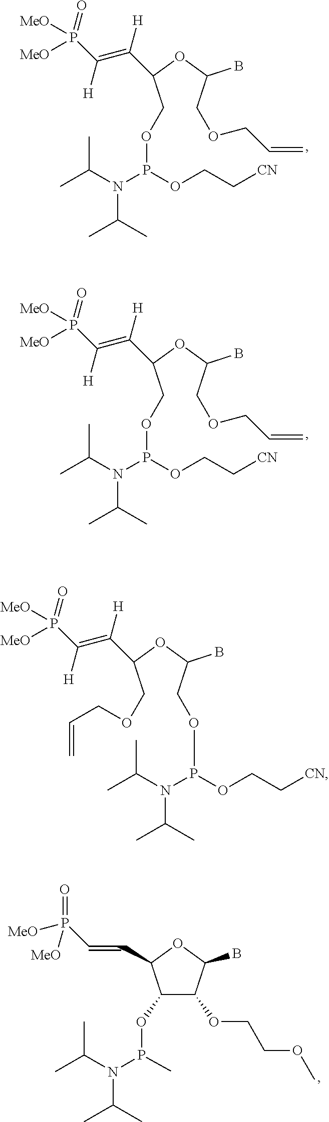 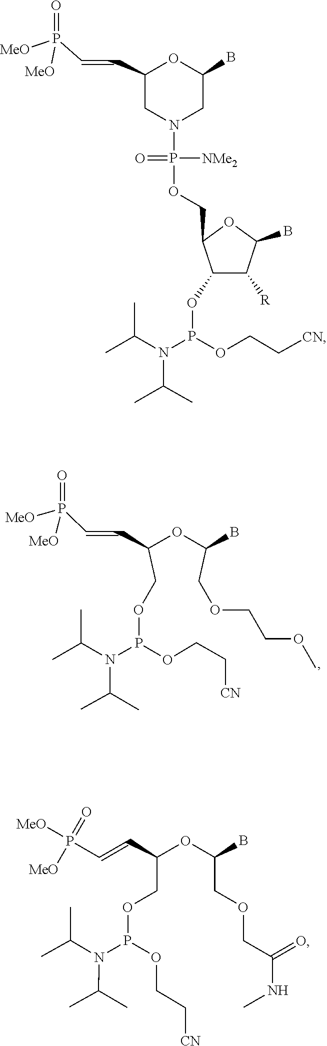 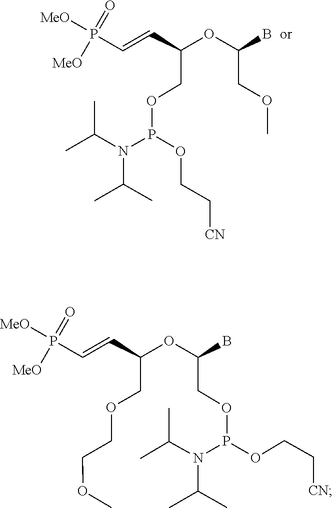
wherein B is a heterocyclic base moiety.


SEQUENCE LISTING (1277 sequences)

SEQ ID NO: 1 - RNA, 23 nt, Homo sapiens
  source 1..23: mol_type = genomic RNA
aaatgactgaatataaacttgtg

SEQ ID NO: 2 - RNA, 23 nt, Homo sapiens
  source 1..23: mol_type = genomic RNA
aatgactgaatataaacttgtgg

SEQ ID NO: 3 - RNA, 23 nt, Homo sapiens
  source 1..23: mol_type = genomic RNA
aacttgtggtagttggagctggt

SEQ ID NO: 4 - RNA, 23 nt, Homo sapiens
  source 1..23: mol_type = genomic RNA
taggcaagagtgccttgacgata

SEQ ID NO: 5 - RNA, 23 nt, Homo sapiens
  source 1..23: mol_type = genomic RNA
ggcaagagtgccttgacgataca

SEQ ID NO: 6 - RNA, 23 nt, Homo sapiens
  source 1..23: mol_type = genomic RNA
gcaagagtgccttgacgatacag

SEQ ID NO: 7 - RNA, 23 nt, Homo sapiens
  source 1..23: mol_type = genomic RNA
caagagtgccttgacgatacagc

SEQ ID NO: 8 - RNA, 23 nt, Homo sapiens
  source 1..23: mol_type = genomic RNA
agtgccttgacgatacagctaat

SEQ ID NO: 9 - RNA, 23 nt, Homo sapiens
  source 1..23: mol_type = genomic RNA
gtgccttgacgatacagctaatt

SEQ ID NO: 10 - RNA, 23 nt, Homo sapiens
  source 1..23: mol_type = genomic RNA
ccttgacgatacagctaattcag

SEQ ID NO: 11 - RNA, 23 nt, Homo sapiens
  source 1..23: mol_type = genomic RNA
cttgacgatacagctaattcaga

SEQ ID NO: 12 - RNA, 23 nt, Homo sapiens
  source 1..23: mol_type = genomic RNA
tacagctaattcagaatcatttt

SEQ ID NO: 13 - RNA, 23 nt, Homo sapiens
  source 1..23: mol_type = genomic RNA
ttgtggacgaatatgatccaaca

SEQ ID NO: 14 - RNA, 23 nt, Homo sapiens
  source 1..23: mol_type = genomic RNA
tggacgaatatgatccaacaata

SEQ ID NO: 15 - RNA, 23 nt, Homo sapiens
  source 1..23: mol_type = genomic RNA
ggacgaatatgatccaacaatag

SEQ ID NO: 16 - DNA, 21 bp, synthetic construct
  source 1..21: mol_type = other DNA
  misc_feature 1..19: note = RNA
  misc_feature 20..21: note = DNA
atgactgaatataaacttgtt

SEQ ID NO: 17 - DNA, 21 bp, synthetic construct
  source 1..21: mol_type = other DNA
  misc_feature 1..19: note = RNA
  misc_feature 20..21: note = DNA
caagtttatattcagtcattt

SEQ ID NO: 18 - DNA, 21 bp, synthetic construct
  source 1..21: mol_type = other DNA
  misc_feature 1..19: note = RNA
  misc_feature 20..21: note = DNA
tgactgaatataaacttgttt

SEQ ID NO: 19 - DNA, 21 bp, synthetic construct
  source 1..21: mol_type = other DNA
  misc_feature 1..19: note = RNA
  misc_feature 20..21: note = DNA
acaagtttatattcagtcatt

SEQ ID NO: 20 - DNA, 21 bp, synthetic construct
  source 1..21: mol_type = other DNA
  misc_feature 1..19: note = RNA
  misc_feature 20..21: note = DNA
cttgtggtagttggagctgtt

SEQ ID NO: 21 - DNA, 21 bp, synthetic construct
  source 1..21: mol_type = other DNA
  misc_feature 1..19: note = RNA
  misc_feature 20..21: note = DNA
cagctccaactaccacaagtt

SEQ ID NO: 22 - DNA, 21 bp, synthetic construct
  source 1..21: mol_type = other DNA
  misc_feature 1..19: note = RNA
  misc_feature 20..21: note = DNA
ggcaagagtgccttgacgatt

SEQ ID NO: 23 - DNA, 21 bp, synthetic construct
  source 1..21: mol_type = other DNA
  misc_feature 1..19: note = RNA
  misc_feature 20..21: note = DNA
tcgtcaaggcactcttgcctt

SEQ ID NO: 24 - DNA, 21 bp, synthetic construct
  source 1..21: mol_type = other DNA
  misc_feature 1..19: note = RNA
  misc_feature 20..21: note = DNA
caagagtgccttgacgatatt

SEQ ID NO: 25 - DNA, 21 bp, synthetic construct
  source 1..21: mol_type = other DNA
  misc_feature 1..19: note = RNA
  misc_feature 20..21: note = DNA
tatcgtcaaggcactcttgtt

SEQ ID NO: 26 - DNA, 21 bp, synthetic construct
  source 1..21: mol_type = other DNA
  misc_feature 1..19: note = RNA
  misc_feature 20..21: note = DNA
aagagtgccttgacgatactt

SEQ ID NO: 27 - DNA, 21 bp, synthetic construct
  source 1..21: mol_type = other DNA
  misc_feature 1..19: note = RNA
  misc_feature 20..21: note = DNA
gtatcgtcaaggcactctttt

SEQ ID NO: 28 - DNA, 21 bp, synthetic construct
  source 1..21: mol_type = other DNA
  misc_feature 1..19: note = RNA
  misc_feature 20..21: note = DNA
agagtgccttgacgatacatt

SEQ ID NO: 29 - DNA, 21 bp, synthetic construct
  source 1..21: mol_type = other DNA
  misc_feature 1..19: note = RNA
  misc_feature 20..21: note = DNA
tgtatcgtcaaggcactcttt

SEQ ID NO: 30 - DNA, 21 bp, synthetic construct
  source 1..21: mol_type = other DNA
  misc_feature 1..19: note = RNA
  misc_feature 20..21: note = DNA
tgccttgacgatacagctatt

SEQ ID NO: 31 - DNA, 21 bp, synthetic construct
  source 1..21: mol_type = other DNA
  misc_feature 1..19: note = RNA
  misc_feature 20..21: note = DNA
tagctgtatcgtcaaggcatt

SEQ ID NO: 32 - DNA, 21 bp, synthetic construct
  source 1..21: mol_type = other DNA
  misc_feature 1..19: note = RNA
  misc_feature 20..21: note = DNA
gccttgacgatacagctaatt

SEQ ID NO: 33 - DNA, 21 bp, synthetic construct
  source 1..21: mol_type = other DNA
  misc_feature 1..19: note = RNA
  misc_feature 20..21: note = DNA
ttagctgtatcgtcaaggctt

SEQ ID NO: 34 - DNA, 21 bp, synthetic construct
  source 1..21: mol_type = other DNA
  misc_feature 1..19: note = RNA
  misc_feature 20..21: note = DNA
ttgacgatacagctaattctt

SEQ ID NO: 35 - DNA, 21 bp, synthetic construct
  source 1..21: mol_type = other DNA
  misc_feature 1..19: note = RNA
  misc_feature 20..21: note = DNA
gaattagctgtatcgtcaatt

SEQ ID NO: 36 - DNA, 21 bp, synthetic construct
  source 1..21: mol_type = other DNA
  misc_feature 1..19: note = RNA
  misc_feature 20..21: note = DNA
tgacgatacagctaattcatt

SEQ ID NO: 37 - DNA, 21 bp, synthetic construct
  source 1..21: mol_type = other DNA
  misc_feature 1..19: note = RNA
  misc_feature 20..21: note = DNA
tgaattagctgtatcgtcatt

SEQ ID NO: 38 - DNA, 21 bp, synthetic construct
  source 1..21: mol_type = other DNA
  misc_feature 1..19: note = RNA
  misc_feature 20..21: note = DNA
cagctaattcagaatcatttt

SEQ ID NO: 39 - DNA, 21 bp, synthetic construct
  source 1..21: mol_type = other DNA
  misc_feature 1..19: note = RNA
  misc_feature 20..21: note = DNA
aatgattctgaattagctgtt

SEQ ID NO: 40 - DNA, 21 bp, synthetic construct
  source 1..21: mol_type = other DNA
  misc_feature 1..19: note = RNA
  misc_feature 20..21: note = DNA
gtggacgaatatgatccaatt

SEQ ID NO: 41 - DNA, 21 bp, synthetic construct
  source 1..21: mol_type = other DNA
  misc_feature 1..19: note = RNA
  misc_feature 20..21: note = DNA
ttggatcatattcgtccactt

SEQ ID NO: 42 - DNA, 21 bp, synthetic construct
  source 1..21: mol_type = other DNA
  misc_feature 1..19: note = RNA
  misc_feature 20..21: note = DNA
gacgaatatgatccaacaatt

SEQ ID NO: 43 - DNA, 21 bp, synthetic construct
  source 1..21: mol_type = other DNA
  misc_feature 1..19: note = RNA
  misc_feature 20..21: note = DNA
ttgttggatcatattcgtctt

SEQ ID NO: 44 - DNA, 21 bp, synthetic construct
  source 1..21: mol_type = other DNA
  misc_feature 1..19: note = RNA
  misc_feature 20..21: note = DNA
acgaatatgatccaacaattt

SEQ ID NO: 45 - DNA, 21 bp, synthetic construct
  source 1..21: mol_type = other DNA
  misc_feature 1..19: note = RNA
  misc_feature 20..21: note = DNA
attgttggatcatattcgttt

SEQ ID NO: 46 - RNA, 23 nt, Homo sapiens
  source 1..23: mol_type = genomic RNA
ggcggccggagtcccgagctagc

SEQ ID NO: 47 - RNA, 23 nt, Homo sapiens
  source 1..23: mol_type = genomic RNA
ggccggagtcccgagctagcccc

SEQ ID NO: 48 - RNA, 23 nt, Homo sapiens
  source 1..23: mol_type = genomic RNA
gccggagtcccgagctagccccg

SEQ ID NO: 49 - RNA, 23 nt, Homo sapiens
  source 1..23: mol_type = genomic RNA
ccggagtcccgagctagccccgg

SEQ ID NO: 50 - RNA, 23 nt, Homo sapiens
  source 1..23: mol_type = genomic RNA
cggagtcccgagctagccccggc

SEQ ID NO: 51 - RNA, 23 nt, Homo sapiens
  source 1..23: mol_type = genomic RNA
ggagtcccgagctagccccggcg

SEQ ID NO: 52 - RNA, 23 nt, Homo sapiens
  source 1..23: mol_type = genomic RNA
gagtcccgagctagccccggcgg

SEQ ID NO: 53 - RNA, 23 nt, Homo sapiens
  source 1..23: mol_type = genomic RNA
gtcccgagctagccccggcggcc

SEQ ID NO: 54 - RNA, 23 nt, Homo sapiens
  source 1..23: mol_type = genomic RNA
ccggacgacaggccacctcgtcg

SEQ ID NO: 55 - RNA, 23 nt, Homo sapiens
  source 1..23: mol_type = genomic RNA
cggacgacaggccacctcgtcgg

SEQ ID NO: 56 - RNA, 23 nt, Homo sapiens
  source 1..23: mol_type = genomic RNA
ggacgacaggccacctcgtcggc

SEQ ID NO: 57 - RNA, 23 nt, Homo sapiens
  source 1..23: mol_type = genomic RNA
gacgacaggccacctcgtcggcg

SEQ ID NO: 58 - RNA, 23 nt, Homo sapiens
  source 1..23: mol_type = genomic RNA
acgacaggccacctcgtcggcgt

SEQ ID NO: 59 - RNA, 23 nt, Homo sapiens
  source 1..23: mol_type = genomic RNA
gacaggccacctcgtcggcgtcc

SEQ ID NO: 60 - RNA, 23 nt, Homo sapiens
  source 1..23: mol_type = genomic RNA
acaggccacctcgtcggcgtccg

SEQ ID NO: 61 - RNA, 23 nt, Homo sapiens
  source 1..23: mol_type = genomic RNA
caggccacctcgtcggcgtccgc

SEQ ID NO: 62 - RNA, 23 nt, Homo sapiens
  source 1..23: mol_type = genomic RNA
aggccacctcgtcggcgtccgcc

SEQ ID NO: 63 - RNA, 23 nt, Homo sapiens
  source 1..23: mol_type = genomic RNA
ggccacctcgtcggcgtccgccc

SEQ ID NO: 64 - RNA, 23 nt, Homo sapiens
  source 1..23: mol_type = genomic RNA
gccacctcgtcggcgtccgcccg

SEQ ID NO: 65 - RNA, 23 nt, Homo sapiens
  source 1..23: mol_type = genomic RNA
ccacctcgtcggcgtccgcccga

SEQ ID NO: 66 - RNA, 23 nt, Homo sapiens
  source 1..23: mol_type = genomic RNA
cacctcgtcggcgtccgcccgag

SEQ ID NO: 67 - RNA, 23 nt, Homo sapiens
  source 1..23: mol_type = genomic RNA
acctcgtcggcgtccgcccgagt

SEQ ID NO: 68 - RNA, 23 nt, Homo sapiens
  source 1..23: mol_type = genomic RNA
cctcgtcggcgtccgcccgagtc

SEQ ID NO: 69 - RNA, 23 nt, Homo sapiens
  source 1..23: mol_type = genomic RNA
ctcgtcggcgtccgcccgagtcc

SEQ ID NO: 70 - RNA, 23 nt, Homo sapiens
  source 1..23: mol_type = genomic RNA
tcgtcggcgtccgcccgagtccc

SEQ ID NO: 71 - RNA, 23 nt, Homo sapiens
  source 1..23: mol_type = genomic RNA
cgtcggcgtccgcccgagtcccc

SEQ ID NO: 72 - RNA, 23 nt, Homo sapiens
  source 1..23: mol_type = genomic RNA
cggcgtccgcccgagtccccgcc

SEQ ID NO: 73 - RNA, 23 nt, Homo sapiens
  source 1..23: mol_type = genomic RNA
ggcgtccgcccgagtccccgcct

SEQ ID NO: 74 - RNA, 23 nt, Homo sapiens
  source 1..23: mol_type = genomic RNA
ccgcccgagtccccgcctcgccg

SEQ ID NO: 75 - RNA, 23 nt, Homo sapiens
  source 1..23: mol_type = genomic RNA
ccaacgccacaaccaccgcgcac

SEQ ID NO: 76 - RNA, 23 nt, Homo sapiens
  source 1..23: mol_type = genomic RNA
caacgccacaaccaccgcgcacg

SEQ ID NO: 77 - RNA, 23 nt, Homo sapiens
  source 1..23: mol_type = genomic RNA
aacgccacaaccaccgcgcacgg

SEQ ID NO: 78 - RNA, 23 nt, Homo sapiens
  source 1..23: mol_type = genomic RNA
cgccacaaccaccgcgcacggcc

SEQ ID NO: 79 - RNA, 23 nt, Homo sapiens
  source 1..23: mol_type = genomic RNA
gccacaaccaccgcgcacggccc

SEQ ID NO: 80 - RNA, 23 nt, Homo sapiens
  source 1..23: mol_type = genomic RNA
ccacaaccaccgcgcacggcccc

SEQ ID NO: 81 - RNA, 23 nt, Homo sapiens
  source 1..23: mol_type = genomic RNA
cgatgcgaccctccgggacggcc

SEQ ID NO: 82 - RNA, 23 nt, Homo sapiens
  source 1..23: mol_type = genomic RNA
gatgcgaccctccgggacggccg

SEQ ID NO: 83 - RNA, 23 nt, Homo sapiens
  source 1..23: mol_type = genomic RNA
atgcgaccctccgggacggccgg

SEQ ID NO: 84 - RNA, 23 nt, Homo sapiens
  source 1..23: mol_type = genomic RNA
gcgaccctccgggacggccgggg

SEQ ID NO: 85 - RNA, 23 nt, Homo sapiens
  source 1..23: mol_type = genomic RNA
cgaccctccgggacggccggggc

SEQ ID NO: 86 - RNA, 23 nt, Homo sapiens
  source 1..23: mol_type = genomic RNA
accctccgggacggccggggcag

SEQ ID NO: 87 - RNA, 23 nt, Homo sapiens
  source 1..23: mol_type = genomic RNA
aaagaaagtttgccaaggcacga

SEQ ID NO: 88 - RNA, 23 nt, Homo sapiens
  source 1..23: mol_type = genomic RNA
aagaaagtttgccaaggcacgag

SEQ ID NO: 89 - RNA, 23 nt, Homo sapiens
  source 1..23: mol_type = genomic RNA
gaaagtttgccaaggcacgagta

SEQ ID NO: 90 - RNA, 23 nt, Homo sapiens
  source 1..23: mol_type = genomic RNA
aaagtttgccaaggcacgagtaa

SEQ ID NO: 91 - RNA, 23 nt, Homo sapiens
  source 1..23: mol_type = genomic RNA
aagtttgccaaggcacgagtaac

SEQ ID NO: 92 - RNA, 23 nt, Homo sapiens
  source 1..23: mol_type = genomic RNA
agtttgccaaggcacgagtaaca

SEQ ID NO: 93 - RNA, 23 nt, Homo sapiens
  source 1..23: mol_type = genomic RNA
gtttgccaaggcacgagtaacaa

SEQ ID NO: 94 - RNA, 23 nt, Homo sapiens
  source 1..23: mol_type = genomic RNA
tttgccaaggcacgagtaacaag

SEQ ID NO: 95 - RNA, 23 nt, Homo sapiens
  source 1..23: mol_type = genomic RNA
ttgccaaggcacgagtaacaagc

SEQ ID NO: 96 - RNA, 23 nt, Homo sapiens
  source 1..23: mol_type = genomic RNA
tcacgcagttgggcacttttgaa

SEQ ID NO: 97 - RNA, 23 nt, Homo sapiens
  source 1..23: mol_type = genomic RNA
cacgcagttgggcacttttgaag

SEQ ID NO: 98 - RNA, 23 nt, Homo sapiens
  source 1..23: mol_type = genomic RNA
acgcagttgggcacttttgaaga

SEQ ID NO: 99 - RNA, 23 nt, Homo sapiens
  source 1..23: mol_type = genomic RNA
cgcagttgggcacttttgaagat

SEQ ID NO: 100 - RNA, 23 nt, Homo sapiens
  source 1..23: mol_type = genomic RNA
gcagttgggcacttttgaagatc

SEQ ID NO: 101 - RNA, 23 nt, Homo sapiens
  source 1..23: mol_type = genomic RNA
cagttgggcacttttgaagatca

SEQ ID NO: 102 - RNA, 23 nt, Homo sapiens
  source 1..23: mol_type = genomic RNA
agttgggcacttttgaagatcat

SEQ ID NO: 103 - RNA, 23 nt, Homo sapiens
  source 1..23: mol_type = genomic RNA
gttgggcacttttgaagatcatt

SEQ ID NO: 104 - RNA, 23 nt, Homo sapiens
  source 1..23: mol_type = genomic RNA
ttgggcacttttgaagatcattt

SEQ ID NO: 105 - RNA, 23 nt, Homo sapiens
  source 1..23: mol_type = genomic RNA
ttttgaagatcattttctcagcc

SEQ ID NO: 106 - RNA, 23 nt, Homo sapiens
  source 1..23: mol_type = genomic RNA
ttgaagatcattttctcagcctc

SEQ ID NO: 107 - RNA, 23 nt, Homo sapiens
  source 1..23: mol_type = genomic RNA
tgaagatcattttctcagcctcc

SEQ ID NO: 108 - RNA, 23 nt, Homo sapiens
  source 1..23: mol_type = genomic RNA
atcattttctcagcctccagagg

SEQ ID NO: 109 - RNA, 23 nt, Homo sapiens
  source 1..23: mol_type = genomic RNA
tcagcctccagaggatgttcaat

SEQ ID NO: 110 - RNA, 23 nt, Homo sapiens
  source 1..23: mol_type = genomic RNA
agcctccagaggatgttcaataa

SEQ ID NO: 111 - RNA, 23 nt, Homo sapiens
  source 1..23: mol_type = genomic RNA
gcctccagaggatgttcaataac

SEQ ID NO: 112 - RNA, 23 nt, Homo sapiens
  source 1..23: mol_type = genomic RNA
ccagaggatgttcaataactgtg

SEQ ID NO: 113 - RNA, 23 nt, Homo sapiens
  source 1..23: mol_type = genomic RNA
agaggatgttcaataactgtgag

SEQ ID NO: 114 - RNA, 23 nt, Homo sapiens
  source 1..23: mol_type = genomic RNA
gatgttcaataactgtgaggtgg

SEQ ID NO: 115 - RNA, 23 nt, Homo sapiens
  source 1..23: mol_type = genomic RNA
tgttcaataactgtgaggtggtc

SEQ ID NO: 116 - RNA, 23 nt, Homo sapiens
  source 1..23: mol_type = genomic RNA
gttcaataactgtgaggtggtcc

SEQ ID NO: 117 - RNA, 23 nt, Homo sapiens
  source 1..23: mol_type = genomic RNA
ttcaataactgtgaggtggtcct

SEQ ID NO: 118 - RNA, 23 nt, Homo sapiens
  source 1..23: mol_type = genomic RNA
tcaataactgtgaggtggtcctt

SEQ ID NO: 119 - RNA, 23 nt, Homo sapiens
  source 1..23: mol_type = genomic RNA
caataactgtgaggtggtccttg

SEQ ID NO: 120 - RNA, 23 nt, Homo sapiens
  source 1..23: mol_type = genomic RNA
aataactgtgaggtggtccttgg

SEQ ID NO: 121 - RNA, 23 nt, Homo sapiens
  source 1..23: mol_type = genomic RNA
taactgtgaggtggtccttggga

SEQ ID NO: 122 - RNA, 23 nt, Homo sapiens
  source 1..23: mol_type = genomic RNA
actgtgaggtggtccttgggaat

SEQ ID NO: 123 - RNA, 23 nt, Homo sapiens
  source 1..23: mol_type = genomic RNA
ctgtgaggtggtccttgggaatt

SEQ ID NO: 124 - RNA, 23 nt, Homo sapiens
  source 1..23: mol_type = genomic RNA
ggtggtccttgggaatttggaaa

SEQ ID NO: 125 - RNA, 23 nt, Homo sapiens
  source 1..23: mol_type = genomic RNA
ccttgggaatttggaaattacct

SEQ ID NO: 126 - RNA, 23 nt, Homo sapiens
  source 1..23: mol_type = genomic RNA
cttgggaatttggaaattaccta

SEQ ID NO: 127 - RNA, 23 nt, Homo sapiens
  source 1..23: mol_type = genomic RNA
ttgggaatttggaaattacctat

SEQ ID NO: 128 - RNA, 23 nt, Homo sapiens
  source 1..23: mol_type = genomic RNA
tgggaatttggaaattacctatg

SEQ ID NO: 129 - RNA, 23 nt, Homo sapiens
  source 1..23: mol_type = genomic RNA
gcagaggaattatgatctttcct

SEQ ID NO: 130 - RNA, 23 nt, Homo sapiens
  source 1..23: mol_type = genomic RNA
cagaggaattatgatctttcctt

SEQ ID NO: 131 - RNA, 23 nt, Homo sapiens
  source 1..23: mol_type = genomic RNA
ggaattatgatctttccttctta

SEQ ID NO: 132 - RNA, 23 nt, Homo sapiens
  source 1..23: mol_type = genomic RNA
gaattatgatctttccttcttaa

SEQ ID NO: 133 - RNA, 23 nt, Homo sapiens
  source 1..23: mol_type = genomic RNA
attatgatctttccttcttaaag

SEQ ID NO: 134 - RNA, 23 nt, Homo sapiens
  source 1..23: mol_type = genomic RNA
tatgatctttccttcttaaagac

SEQ ID NO: 135 - RNA, 23 nt, Homo sapiens
  source 1..23: mol_type = genomic RNA
gatctttccttcttaaagaccat

SEQ ID NO: 136 - RNA, 23 nt, Homo sapiens
  source 1..23: mol_type = genomic RNA
ttccttcttaaagaccatccagg

SEQ ID NO: 137 - RNA, 23 nt, Homo sapiens
  source 1..23: mol_type = genomic RNA
tccttcttaaagaccatccagga

SEQ ID NO: 138 - RNA, 23 nt, Homo sapiens
  source 1..23: mol_type = genomic RNA
cttcttaaagaccatccaggagg

SEQ ID NO: 139 - RNA, 23 nt, Homo sapiens
  source 1..23: mol_type = genomic RNA
tcttaaagaccatccaggaggtg

SEQ ID NO: 140 - RNA, 23 nt, Homo sapiens
  source 1..23: mol_type = genomic RNA
cttaaagaccatccaggaggtgg

SEQ ID NO: 141 - RNA, 23 nt, Homo sapiens
  source 1..23: mol_type = genomic RNA
atccaggaggtggctggttatgt

SEQ ID NO: 142 - RNA, 23 nt, Homo sapiens
  source 1..23: mol_type = genomic RNA
tccaggaggtggctggttatgtc

SEQ ID NO: 143 - RNA, 23 nt, Homo sapiens
  source 1..23: mol_type = genomic RNA
ccaggaggtggctggttatgtcc

SEQ ID NO: 144 - RNA, 23 nt, Homo sapiens
  source 1..23: mol_type = genomic RNA
caggaggtggctggttatgtcct

SEQ ID NO: 145 - RNA, 23 nt, Homo sapiens
  source 1..23: mol_type = genomic RNA
aggaggtggctggttatgtcctc

SEQ ID NO: 146 - RNA, 23 nt, Homo sapiens
  source 1..23: mol_type = genomic RNA
ggaggtggctggttatgtcctca

SEQ ID NO: 147 - RNA, 23 nt, Homo sapiens
  source 1..23: mol_type = genomic RNA
aggtggctggttatgtcctcatt

SEQ ID NO: 148 - RNA, 23 nt, Homo sapiens
  source 1..23: mol_type = genomic RNA
ttgccctcaacacagtggagcga

SEQ ID NO: 149 - RNA, 23 nt, Homo sapiens
  source 1..23: mol_type = genomic RNA
aattcctttggaaaacctgcaga

SEQ ID NO: 150 - RNA, 23 nt, Homo sapiens
  source 1..23: mol_type = genomic RNA
attcctttggaaaacctgcagat

SEQ ID NO: 151 - RNA, 23 nt, Homo sapiens
  source 1..23: mol_type = genomic RNA
tggaaaacctgcagatcatcaga

SEQ ID NO: 152 - RNA, 23 nt, Homo sapiens
  source 1..23: mol_type = genomic RNA
ggaaaacctgcagatcatcagag

SEQ ID NO: 153 - RNA, 23 nt, Homo sapiens
  source 1..23: mol_type = genomic RNA
aaaacctgcagatcatcagagga

SEQ ID NO: 154 - RNA, 23 nt, Homo sapiens
  source 1..23: mol_type = genomic RNA
acctgcagatcatcagaggaaat

SEQ ID NO: 155 - RNA, 23 nt, Homo sapiens
  source 1..23: mol_type = genomic RNA
acgaaaattcctatgccttagca

SEQ ID NO: 156 - RNA, 23 nt, Homo sapiens
  source 1..23: mol_type = genomic RNA
cgaaaattcctatgccttagcag

SEQ ID NO: 157 - RNA, 23 nt, Homo sapiens
  source 1..23: mol_type = genomic RNA
aaaattcctatgccttagcagtc

SEQ ID NO: 158 - RNA, 23 nt, Homo sapiens
  source 1..23: mol_type = genomic RNA
attcctatgccttagcagtctta

SEQ ID NO: 159 - RNA, 23 nt, Homo sapiens
  source 1..23: mol_type = genomic RNA
ttcctatgccttagcagtcttat

SEQ ID NO: 160 - RNA, 23 nt, Homo sapiens
  source 1..23: mol_type = genomic RNA
tcctatgccttagcagtcttatc

SEQ ID NO: 161 - RNA, 23 nt, Homo sapiens
  source 1..23: mol_type = genomic RNA
ctatgccttagcagtcttatcta

SEQ ID NO: 162 - RNA, 23 nt, Homo sapiens
  source 1..23: mol_type = genomic RNA
tatgccttagcagtcttatctaa

SEQ ID NO: 163 - RNA, 23 nt, Homo sapiens
  source 1..23: mol_type = genomic RNA
atgccttagcagtcttatctaac

SEQ ID NO: 164 - RNA, 23 nt, Homo sapiens
  source 1..23: mol_type = genomic RNA
tgccttagcagtcttatctaact

SEQ ID NO: 165 - RNA, 23 nt, Homo sapiens
  source 1..23: mol_type = genomic RNA
gccttagcagtcttatctaacta

SEQ ID NO: 166 - RNA, 23 nt, Homo sapiens
  source 1..23: mol_type = genomic RNA
ccttagcagtcttatctaactat

SEQ ID NO: 167 - RNA, 23 nt, Homo sapiens
  source 1..23: mol_type = genomic RNA
cttagcagtcttatctaactatg

SEQ ID NO: 168 - RNA, 23 nt, Homo sapiens
  source 1..23: mol_type = genomic RNA
ttagcagtcttatctaactatga

SEQ ID NO: 169 - RNA, 23 nt, Homo sapiens
  source 1..23: mol_type = genomic RNA
tagcagtcttatctaactatgat

SEQ ID NO: 170 - RNA, 23 nt, Homo sapiens
  source 1..23: mol_type = genomic RNA
agcagtcttatctaactatgatg

SEQ ID NO: 171 - RNA, 23 nt, Homo sapiens
  source 1..23: mol_type = genomic RNA
agtcttatctaactatgatgcaa

SEQ ID NO: 172 - RNA, 23 nt, Homo sapiens
  source 1..23: mol_type = genomic RNA
gtcttatctaactatgatgcaaa

SEQ ID NO: 173 - RNA, 23 nt, Homo sapiens
  source 1..23: mol_type = genomic RNA
tcttatctaactatgatgcaaat

SEQ ID NO: 174 - RNA, 23 nt, Homo sapiens
  source 1..23: mol_type = genomic RNA
cttatctaactatgatgcaaata

SEQ ID NO: 175 - RNA, 23 nt, Homo sapiens
  source 1..23: mol_type = genomic RNA
ttatctaactatgatgcaaataa

SEQ ID NO: 176 - RNA, 23 nt, Homo sapiens
  source 1..23: mol_type = genomic RNA
tatctaactatgatgcaaataaa

SEQ ID NO: 177 - RNA, 23 nt, Homo sapiens
  source 1..23: mol_type = genomic RNA
atctaactatgatgcaaataaaa

SEQ ID NO: 178 - RNA, 23 nt, Homo sapiens
  source 1..23: mol_type = genomic RNA
ctaactatgatgcaaataaaacc

SEQ ID NO: 179 - RNA, 23 nt, Homo sapiens
  source 1..23: mol_type = genomic RNA
atgatgcaaataaaaccggactg

SEQ ID NO: 180 - RNA, 23 nt, Homo sapiens
  source 1..23: mol_type = genomic RNA
tgatgcaaataaaaccggactga

SEQ ID NO: 181 - RNA, 23 nt, Homo sapiens
  source 1..23: mol_type = genomic RNA
gatgcaaataaaaccggactgaa

SEQ ID NO: 182 - RNA, 23 nt, Homo sapiens
  source 1..23: mol_type = genomic RNA
tgcaaataaaaccggactgaagg

SEQ ID NO: 183 - RNA, 23 nt, Homo sapiens
  source 1..23: mol_type = genomic RNA
gcaaataaaaccggactgaagga

SEQ ID NO: 184 - RNA, 23 nt, Homo sapiens
  source 1..23: mol_type = genomic RNA
caaataaaaccggactgaaggag

SEQ ID NO: 185 - RNA, 23 nt, Homo sapiens
  source 1..23: mol_type = genomic RNA
aataaaaccggactgaaggagct

SEQ ID NO: 186 - RNA, 23 nt, Homo sapiens
  source 1..23: mol_type = genomic RNA
ataaaaccggactgaaggagctg

SEQ ID NO: 187 - RNA, 23 nt, Homo sapiens
  source 1..23: mol_type = genomic RNA
taaaaccggactgaaggagctgc

SEQ ID NO: 188 - RNA, 23 nt, Homo sapiens
  source 1..23: mol_type = genomic RNA
aaaaccggactgaaggagctgcc

SEQ ID NO: 189 - RNA, 23 nt, Homo sapiens
  source 1..23: mol_type = genomic RNA
gaaggagctgcccatgagaaatt

SEQ ID NO: 190 - RNA, 23 nt, Homo sapiens
  source 1..23: mol_type = genomic RNA
tttacaggaaatcctgcatggcg

SEQ ID NO: 191 - RNA, 23 nt, Homo sapiens
  source 1..23: mol_type = genomic RNA
acaggaaatcctgcatggcgccg

SEQ ID NO: 192 - RNA, 23 nt, Homo sapiens
  source 1..23: mol_type = genomic RNA
caggaaatcctgcatggcgccgt

SEQ ID NO: 193 - RNA, 23 nt, Homo sapiens
  source 1..23: mol_type = genomic RNA
aggaaatcctgcatggcgccgtg

SEQ ID NO: 194 - RNA, 23 nt, Homo sapiens
  source 1..23: mol_type = genomic RNA
ggaaatcctgcatggcgccgtgc

SEQ ID NO: 195 - RNA, 23 nt, Homo sapiens
  source 1..23: mol_type = genomic RNA
gaaatcctgcatggcgccgtgcg

SEQ ID NO: 196 - RNA, 23 nt, Homo sapiens
  source 1..23: mol_type = genomic RNA
aatcctgcatggcgccgtgcggt

SEQ ID NO: 197 - RNA, 23 nt, Homo sapiens
  source 1..23: mol_type = genomic RNA
tcctgcatggcgccgtgcggttc

SEQ ID NO: 198 - RNA, 23 nt, Homo sapiens
  source 1..23: mol_type = genomic RNA
cctgcatggcgccgtgcggttca

SEQ ID NO: 199 - RNA, 23 nt, Homo sapiens
  source 1..23: mol_type = genomic RNA
ctgcatggcgccgtgcggttcag

SEQ ID NO: 200 - RNA, 23 nt, Homo sapiens
  source 1..23: mol_type = genomic RNA
gcatggcgccgtgcggttcagca

SEQ ID NO: 201 - RNA, 23 nt, Homo sapiens
  source 1..23: mol_type = genomic RNA
catggcgccgtgcggttcagcaa

SEQ ID NO: 202 - RNA, 23 nt, Homo sapiens
  source 1..23: mol_type = genomic RNA
atggcgccgtgcggttcagcaac

SEQ ID NO: 203 - RNA, 23 nt, Homo sapiens
  source 1..23: mol_type = genomic RNA
tggcgccgtgcggttcagcaaca

SEQ ID NO: 204 - RNA, 23 nt, Homo sapiens
  source 1..23: mol_type = genomic RNA
ggcgccgtgcggttcagcaacaa

SEQ ID NO: 205 - RNA, 23 nt, Homo sapiens
  source 1..23: mol_type = genomic RNA
gcgccgtgcggttcagcaacaac

SEQ ID NO: 206 - RNA, 23 nt, Homo sapiens
  source 1..23: mol_type = genomic RNA
cgccgtgcggttcagcaacaacc

SEQ ID NO: 207 - RNA, 23 nt, Homo sapiens
  source 1..23: mol_type = genomic RNA
ccgtgcggttcagcaacaaccct

SEQ ID NO: 208 - RNA, 23 nt, Homo sapiens
  source 1..23: mol_type = genomic RNA
gtgcggttcagcaacaaccctgc

SEQ ID NO: 209 - RNA, 23 nt, Homo sapiens
  source 1..23: mol_type = genomic RNA
gcggttcagcaacaaccctgccc

SEQ ID NO: 210 - RNA, 23 nt, Homo sapiens
  source 1..23: mol_type = genomic RNA
cagcaacaaccctgccctgtgca

SEQ ID NO: 211 - RNA, 23 nt, Homo sapiens
  source 1..23: mol_type = genomic RNA
gcaacaaccctgccctgtgcaac

SEQ ID NO: 212 - RNA, 23 nt, Homo sapiens
  source 1..23: mol_type = genomic RNA
caacgtggagagcatccagtggc

SEQ ID NO: 213 - RNA, 23 nt, Homo sapiens
  source 1..23: mol_type = genomic RNA
aacgtggagagcatccagtggcg

SEQ ID NO: 214 - RNA, 23 nt, Homo sapiens
  source 1..23: mol_type = genomic RNA
acgtggagagcatccagtggcgg

SEQ ID NO: 215 - RNA, 23 nt, Homo sapiens
  source 1..23: mol_type = genomic RNA
tggagagcatccagtggcgggac

SEQ ID NO: 216 - RNA, 23 nt, Homo sapiens
  source 1..23: mol_type = genomic RNA
ggagagcatccagtggcgggaca

SEQ ID NO: 217 - RNA, 23 nt, Homo sapiens
  source 1..23: mol_type = genomic RNA
gagagcatccagtggcgggacat

SEQ ID NO: 218 - RNA, 23 nt, Homo sapiens
  source 1..23: mol_type = genomic RNA
tccagtggcgggacatagtcagc

SEQ ID NO: 219 - RNA, 23 nt, Homo sapiens
  source 1..23: mol_type = genomic RNA
ccagtggcgggacatagtcagca

SEQ ID NO: 220 - RNA, 23 nt, Homo sapiens
  source 1..23: mol_type = genomic RNA
agtggcgggacatagtcagcagt

SEQ ID NO: 221 - RNA, 23 nt, Homo sapiens
  source 1..23: mol_type = genomic RNA
gtggcgggacatagtcagcagtg

SEQ ID NO: 222 - RNA, 23 nt, Homo sapiens
  source 1..23: mol_type = genomic RNA
ttctcagcaacatgtcgatggac

SEQ ID NO: 223 - RNA, 23 nt, Homo sapiens
  source 1..23: mol_type = genomic RNA
ctcagcaacatgtcgatggactt

SEQ ID NO: 224 - RNA, 23 nt, Homo sapiens
  source 1..23: mol_type = genomic RNA
tcagcaacatgtcgatggacttc

SEQ ID NO: 225 - RNA, 23 nt, Homo sapiens
  source 1..23: mol_type = genomic RNA
cagcaacatgtcgatggacttcc

SEQ ID NO: 226 - RNA, 23 nt, Homo sapiens
  source 1..23: mol_type = genomic RNA
gcaacatgtcgatggacttccag

SEQ ID NO: 227 - RNA, 23 nt, Homo sapiens
  source 1..23: mol_type = genomic RNA
caacatgtcgatggacttccaga

SEQ ID NO: 228 - RNA, 23 nt, Homo sapiens
  source 1..23: mol_type = genomic RNA
aacatgtcgatggacttccagaa

SEQ ID NO: 229 - RNA, 23 nt, Homo sapiens
  source 1..23: mol_type = genomic RNA
acatgtcgatggacttccagaac

SEQ ID NO: 230 - RNA, 23 nt, Homo sapiens
  source 1..23: mol_type = genomic RNA
tgtcgatggacttccagaaccac

SEQ ID NO: 231 - RNA, 23 nt, Homo sapiens
  source 1..23: mol_type = genomic RNA
cagaaccacctgggcagctgcca

SEQ ID NO: 232 - RNA, 23 nt, Homo sapiens
  source 1..23: mol_type = genomic RNA
ctgggcagctgccaaaagtgtga

SEQ ID NO: 233 - RNA, 23 nt, Homo sapiens
  source 1..23: mol_type = genomic RNA
gggcagctgccaaaagtgtgatc

SEQ ID NO: 234 - RNA, 23 nt, Homo sapiens
  source 1..23: mol_type = genomic RNA
gctgccaaaagtgtgatccaagc

SEQ ID NO: 235 - RNA, 23 nt, Homo sapiens
  source 1..23: mol_type = genomic RNA
ctgccaaaagtgtgatccaagct

SEQ ID NO: 236 - RNA, 23 nt, Homo sapiens
  source 1..23: mol_type = genomic RNA
tgccaaaagtgtgatccaagctg

SEQ ID NO: 237 - RNA, 23 nt, Homo sapiens
  source 1..23: mol_type = genomic RNA
caaaagtgtgatccaagctgtcc

SEQ ID NO: 238 - RNA, 23 nt, Homo sapiens
  source 1..23: mol_type = genomic RNA
agtgtgatccaagctgtcccaat

SEQ ID NO: 239 - RNA, 23 nt, Homo sapiens
  source 1..23: mol_type = genomic RNA
gtgtgatccaagctgtcccaatg

SEQ ID NO: 240 - RNA, 23 nt, Homo sapiens
  source 1..23: mol_type = genomic RNA
gtgatccaagctgtcccaatggg

SEQ ID NO: 241 - RNA, 23 nt, Homo sapiens
  source 1..23: mol_type = genomic RNA
tgatccaagctgtcccaatggga

SEQ ID NO: 242 - RNA, 23 nt, Homo sapiens
  source 1..23: mol_type = genomic RNA
gatccaagctgtcccaatgggag

SEQ ID NO: 243 - RNA, 23 nt, Homo sapiens
  source 1..23: mol_type = genomic RNA
atccaagctgtcccaatgggagc

SEQ ID NO: 244 - RNA, 23 nt, Homo sapiens
  source 1..23: mol_type = genomic RNA
tccaagctgtcccaatgggagct

SEQ ID NO: 245 - RNA, 23 nt, Homo sapiens
  source 1..23: mol_type = genomic RNA
caagctgtcccaatgggagctgc

SEQ ID NO: 246 - RNA, 23 nt, Homo sapiens
  source 1..23: mol_type = genomic RNA
gctgtcccaatgggagctgctgg

SEQ ID NO: 247 - RNA, 23 nt, Homo sapiens
  source 1..23: mol_type = genomic RNA
ggggtgcaggagaggagaactgc

SEQ ID NO: 248 - RNA, 23 nt, Homo sapiens
  source 1..23: mol_type = genomic RNA
agaaactgaccaaaatcatctgt

SEQ ID NO: 249 - RNA, 23 nt, Homo sapiens
  source 1..23: mol_type = genomic RNA
gaaactgaccaaaatcatctgtg

SEQ ID NO: 250 - RNA, 23 nt, Homo sapiens
  source 1..23: mol_type = genomic RNA
aaactgaccaaaatcatctgtgc

SEQ ID NO: 251 - RNA, 23 nt, Homo sapiens
  source 1..23: mol_type = genomic RNA
tgaccaaaatcatctgtgcccag

SEQ ID NO: 252 - RNA, 23 nt, Homo sapiens
  source 1..23: mol_type = genomic RNA
gaccaaaatcatctgtgcccagc

SEQ ID NO: 253 - RNA, 23 nt, Homo sapiens
  source 1..23: mol_type = genomic RNA
caaaatcatctgtgcccagcagt

SEQ ID NO: 254 - RNA, 23 nt, Homo sapiens
  source 1..23: mol_type = genomic RNA
ctgtgcccagcagtgctccgggc

SEQ ID NO: 255 - RNA, 23 nt, Homo sapiens
  source 1..23: mol_type = genomic RNA
gtgcccagcagtgctccgggcgc

SEQ ID NO: 256 - RNA, 23 nt, Homo sapiens
  source 1..23: mol_type = genomic RNA
ccccagtgactgctgccacaacc

SEQ ID NO: 257 - RNA, 23 nt, Homo sapiens
  source 1..23: mol_type = genomic RNA
cccagtgactgctgccacaacca

SEQ ID NO: 258 - RNA, 23 nt, Homo sapiens
  source 1..23: mol_type = genomic RNA
gggagagcgactgcctggtctgc

SEQ ID NO: 259 - RNA, 23 nt, Homo sapiens
  source 1..23: mol_type = genomic RNA
ggagagcgactgcctggtctgcc

SEQ ID NO: 260 - RNA, 23 nt, Homo sapiens
  source 1..23: mol_type = genomic RNA
gagagcgactgcctggtctgccg

SEQ ID NO: 261 - RNA, 23 nt, Homo sapiens
  source 1..23: mol_type = genomic RNA
agagcgactgcctggtctgccgc

SEQ ID NO: 262 - RNA, 23 nt, Homo sapiens
  source 1..23: mol_type = genomic RNA
gagcgactgcctggtctgccgca

SEQ ID NO: 263 - RNA, 23 nt, Homo sapiens
  source 1..23: mol_type = genomic RNA
agcgactgcctggtctgccgcaa

SEQ ID NO: 264 - RNA, 23 nt, Homo sapiens
  source 1..23: mol_type = genomic RNA
ctgcctggtctgccgcaaattcc

SEQ ID NO: 265 - RNA, 23 nt, Homo sapiens
  source 1..23: mol_type = genomic RNA
tgcctggtctgccgcaaattccg

SEQ ID NO: 266 - RNA, 23 nt, Homo sapiens
  source 1..23: mol_type = genomic RNA
gcctggtctgccgcaaattccga

SEQ ID NO: 267 - RNA, 23 nt, Homo sapiens
  source 1..23: mol_type = genomic RNA
cctggtctgccgcaaattccgag

SEQ ID NO: 268 - RNA, 23 nt, Homo sapiens
  source 1..23: mol_type = genomic RNA
tggtctgccgcaaattccgagac

SEQ ID NO: 269 - RNA, 23 nt, Homo sapiens
  source 1..23: mol_type = genomic RNA
ggtctgccgcaaattccgagacg

SEQ ID NO: 270 - RNA, 23 nt, Homo sapiens
  source 1..23: mol_type = genomic RNA
gtctgccgcaaattccgagacga

SEQ ID NO: 271 - RNA, 23 nt, Homo sapiens
  source 1..23: mol_type = genomic RNA
tctgccgcaaattccgagacgaa

SEQ ID NO: 272 - RNA, 23 nt, Homo sapiens
  source 1..23: mol_type = genomic RNA
tgccgcaaattccgagacgaagc

SEQ ID NO: 273 - RNA, 23 nt, Homo sapiens
  source 1..23: mol_type = genomic RNA
caaattccgagacgaagccacgt

SEQ ID NO: 274 - RNA, 23 nt, Homo sapiens
  source 1..23: mol_type = genomic RNA
aaattccgagacgaagccacgtg

SEQ ID NO: 275 - RNA, 23 nt, Homo sapiens
  source 1..23: mol_type = genomic RNA
aattccgagacgaagccacgtgc

SEQ ID NO: 276 - RNA, 23 nt, Homo sapiens
  source 1..23: mol_type = genomic RNA
attccgagacgaagccacgtgca

SEQ ID NO: 277 - RNA, 23 nt, Homo sapiens
  source 1..23: mol_type = genomic RNA
ttccgagacgaagccacgtgcaa

SEQ ID NO: 278 - RNA, 23 nt, Homo sapiens
  source 1..23: mol_type = genomic RNA
ccgagacgaagccacgtgcaagg

SEQ ID NO: 279 - RNA, 23 nt, Homo sapiens
  source 1..23: mol_type = genomic RNA
agacgaagccacgtgcaaggaca

SEQ ID NO: 280 - RNA, 23 nt, Homo sapiens
  source 1..23: mol_type = genomic RNA
gacgaagccacgtgcaaggacac

SEQ ID NO: 281 - RNA, 23 nt, Homo sapiens
  source 1..23: mol_type = genomic RNA
acgaagccacgtgcaaggacacc

SEQ ID NO: 282 - RNA, 23 nt, Homo sapiens
  source 1..23: mol_type = genomic RNA
cgaagccacgtgcaaggacacct

SEQ ID NO: 283 - RNA, 23 nt, Homo sapiens
  source 1..23: mol_type = genomic RNA
ccccccactcatgctctacaacc

SEQ ID NO: 284 - RNA, 23 nt, Homo sapiens
  source 1..23: mol_type = genomic RNA
ccccactcatgctctacaacccc

SEQ ID NO: 285 - RNA, 23 nt, Homo sapiens
  source 1..23: mol_type = genomic RNA
ccactcatgctctacaaccccac

SEQ ID NO: 286 - RNA, 23 nt, Homo sapiens
  source 1..23: mol_type = genomic RNA
ctcatgctctacaaccccaccac

SEQ ID NO: 287 - RNA, 23 nt, Homo sapiens
  source 1..23: mol_type = genomic RNA
taccagatggatgtgaaccccga

SEQ ID NO: 288 - RNA, 23 nt, Homo sapiens
  source 1..23: mol_type = genomic RNA
ccagatggatgtgaaccccgagg

SEQ ID NO: 289 - RNA, 23 nt, Homo sapiens
  source 1..23: mol_type = genomic RNA
cagatggatgtgaaccccgaggg

SEQ ID NO: 290 - RNA, 23 nt, Homo sapiens
  source 1..23: mol_type = genomic RNA
agatggatgtgaaccccgagggc

SEQ ID NO: 291 - RNA, 23 nt, Homo sapiens
  source 1..23: mol_type = genomic RNA
atggatgtgaaccccgagggcaa

SEQ ID NO: 292 - RNA, 23 nt, Homo sapiens
  source 1..23: mol_type = genomic RNA
tggatgtgaaccccgagggcaaa

SEQ ID NO: 293 - RNA, 23 nt, Homo sapiens
  source 1..23: mol_type = genomic RNA
gatgtgaaccccgagggcaaata

SEQ ID NO: 294 - RNA, 23 nt, Homo sapiens
  source 1..23: mol_type = genomic RNA
tgaaccccgagggcaaatacagc

SEQ ID NO: 295 - RNA, 23 nt, Homo sapiens
  source 1..23: mol_type = genomic RNA
gaaccccgagggcaaatacagct

SEQ ID NO: 296 - RNA, 23 nt, Homo sapiens
  source 1..23: mol_type = genomic RNA
accccgagggcaaatacagcttt

SEQ ID NO: 297 - RNA, 23 nt, Homo sapiens
  source 1..23: mol_type = genomic RNA
ccccgagggcaaatacagctttg

SEQ ID NO: 298 - RNA, 23 nt, Homo sapiens
  source 1..23: mol_type = genomic RNA
cccgagggcaaatacagctttgg

SEQ ID NO: 299 - RNA, 23 nt, Homo sapiens
  source 1..23: mol_type = genomic RNA
gcaaatacagctttggtgccacc

SEQ ID NO: 300 - RNA, 23 nt, Homo sapiens
  source 1..23: mol_type = genomic RNA
caaatacagctttggtgccacct

SEQ ID NO: 301 - RNA, 23 nt, Homo sapiens
  source 1..23: mol_type = genomic RNA
aaatacagctttggtgccacctg

SEQ ID NO: 302 - RNA, 23 nt, Homo sapiens
  source 1..23: mol_type = genomic RNA
agctttggtgccacctgcgtgaa

SEQ ID NO: 303 - RNA, 23 nt, Homo sapiens
  source 1..23: mol_type = genomic RNA
ctttggtgccacctgcgtgaaga

SEQ ID NO: 304 - RNA, 23 nt, Homo sapiens
  source 1..23: mol_type = genomic RNA
tgccacctgcgtgaagaagtgtc

SEQ ID NO: 305 - RNA, 23 nt, Homo sapiens
  source 1..23: mol_type = genomic RNA
acctgcgtgaagaagtgtccccg

SEQ ID NO: 306 - RNA, 23 nt, Homo sapiens
  source 1..23: mol_type = genomic RNA
cctgcgtgaagaagtgtccccgt

SEQ ID NO: 307 - RNA, 23 nt, Homo sapiens
  source 1..23: mol_type = genomic RNA
ctgcgtgaagaagtgtccccgta

SEQ ID NO: 308 - RNA, 23 nt, Homo sapiens
  source 1..23: mol_type = genomic RNA
tgcgtgaagaagtgtccccgtaa

SEQ ID NO: 309 - RNA, 23 nt, Homo sapiens
  source 1..23: mol_type = genomic RNA
gcgtgaagaagtgtccccgtaat

SEQ ID NO: 310 - RNA, 23 nt, Homo sapiens
  source 1..23: mol_type = genomic RNA
cgtgaagaagtgtccccgtaatt

SEQ ID NO: 311 - RNA, 23 nt, Homo sapiens
  source 1..23: mol_type = genomic RNA
gtgaagaagtgtccccgtaatta

SEQ ID NO: 312 - RNA, 23 nt, Homo sapiens
  source 1..23: mol_type = genomic RNA
tgaagaagtgtccccgtaattat

SEQ ID NO: 313 - RNA, 23 nt, Homo sapiens
  source 1..23: mol_type = genomic RNA
gaagaagtgtccccgtaattatg

SEQ ID NO: 314 - RNA, 23 nt, Homo sapiens
  source 1..23: mol_type = genomic RNA
aagaagtgtccccgtaattatgt

SEQ ID NO: 315 - RNA, 23 nt, Homo sapiens
  source 1..23: mol_type = genomic RNA
agaagtgtccccgtaattatgtg

SEQ ID NO: 316 - RNA, 23 nt, Homo sapiens
  source 1..23: mol_type = genomic RNA
gaagtgtccccgtaattatgtgg

SEQ ID NO: 317 - RNA, 23 nt, Homo sapiens
  source 1..23: mol_type = genomic RNA
aagtgtccccgtaattatgtggt

SEQ ID NO: 318 - RNA, 23 nt, Homo sapiens
  source 1..23: mol_type = genomic RNA
agtgtccccgtaattatgtggtg

SEQ ID NO: 319 - RNA, 23 nt, Homo sapiens
  source 1..23: mol_type = genomic RNA
gtgtccccgtaattatgtggtga

SEQ ID NO: 320 - RNA, 23 nt, Homo sapiens
  source 1..23: mol_type = genomic RNA
gtccccgtaattatgtggtgaca

SEQ ID NO: 321 - RNA, 23 nt, Homo sapiens
  source 1..23: mol_type = genomic RNA
ccccgtaattatgtggtgacaga

SEQ ID NO: 322 - RNA, 23 nt, Homo sapiens
  source 1..23: mol_type = genomic RNA
ccgtaattatgtggtgacagatc

SEQ ID NO: 323 - RNA, 23 nt, Homo sapiens
  source 1..23: mol_type = genomic RNA
cgtaattatgtggtgacagatca

SEQ ID NO: 324 - RNA, 23 nt, Homo sapiens
  source 1..23: mol_type = genomic RNA
gtaattatgtggtgacagatcac

SEQ ID NO: 325 - RNA, 23 nt, Homo sapiens
  source 1..23: mol_type = genomic RNA
taattatgtggtgacagatcacg

SEQ ID NO: 326 - RNA, 23 nt, Homo sapiens
  source 1..23: mol_type = genomic RNA
aattatgtggtgacagatcacgg

SEQ ID NO: 327 - RNA, 23 nt, Homo sapiens
  source 1..23: mol_type = genomic RNA
ttatgtggtgacagatcacggct

SEQ ID NO: 328 - RNA, 23 nt, Homo sapiens
  source 1..23: mol_type = genomic RNA
tgtggtgacagatcacggctcgt

SEQ ID NO: 329 - RNA, 23 nt, Homo sapiens
  source 1..23: mol_type = genomic RNA
tggtgacagatcacggctcgtgc

SEQ ID NO: 330 - RNA, 23 nt, Homo sapiens
  source 1..23: mol_type = genomic RNA
ggtgacagatcacggctcgtgcg

SEQ ID NO: 331 - RNA, 23 nt, Homo sapiens
  source 1..23: mol_type = genomic RNA
gtgacagatcacggctcgtgcgt

SEQ ID NO: 332 - RNA, 23 nt, Homo sapiens
  source 1..23: mol_type = genomic RNA
tgacagatcacggctcgtgcgtc

SEQ ID NO: 333 - RNA, 23 nt, Homo sapiens
  source 1..23: mol_type = genomic RNA
gacagatcacggctcgtgcgtcc

SEQ ID NO: 334 - RNA, 23 nt, Homo sapiens
  source 1..23: mol_type = genomic RNA
acagatcacggctcgtgcgtccg

SEQ ID NO: 335 - RNA, 23 nt, Homo sapiens
  source 1..23: mol_type = genomic RNA
cagatcacggctcgtgcgtccga

SEQ ID NO: 336 - RNA, 23 nt, Homo sapiens
  source 1..23: mol_type = genomic RNA
agatcacggctcgtgcgtccgag

SEQ ID NO: 337 - RNA, 23 nt, Homo sapiens
  source 1..23: mol_type = genomic RNA
gatcacggctcgtgcgtccgagc

SEQ ID NO: 338 - RNA, 23 nt, Homo sapiens
  source 1..23: mol_type = genomic RNA
atcacggctcgtgcgtccgagcc

SEQ ID NO: 339 - RNA, 23 nt, Homo sapiens
  source 1..23: mol_type = genomic RNA
tcacggctcgtgcgtccgagcct

SEQ ID NO: 340 - RNA, 23 nt, Homo sapiens
  source 1..23: mol_type = genomic RNA
cggctcgtgcgtccgagcctgtg

SEQ ID NO: 341 - RNA, 23 nt, Homo sapiens
  source 1..23: mol_type = genomic RNA
atggaggaagacggcgtccgcaa

SEQ ID NO: 342 - RNA, 23 nt, Homo sapiens
  source 1..23: mol_type = genomic RNA
tggaggaagacggcgtccgcaag

SEQ ID NO: 343 - RNA, 23 nt, Homo sapiens
  source 1..23: mol_type = genomic RNA
gaggaagacggcgtccgcaagtg

SEQ ID NO: 344 - RNA, 23 nt, Homo sapiens
  source 1..23: mol_type = genomic RNA
aggaagacggcgtccgcaagtgt

SEQ ID NO: 345 - RNA, 23 nt, Homo sapiens
  source 1..23: mol_type = genomic RNA
ggaagacggcgtccgcaagtgta

SEQ ID NO: 346 - RNA, 23 nt, Homo sapiens
  source 1..23: mol_type = genomic RNA
aagacggcgtccgcaagtgtaag

SEQ ID NO: 347 - RNA, 23 nt, Homo sapiens
  source 1..23: mol_type = genomic RNA
agacggcgtccgcaagtgtaaga

SEQ ID NO: 348 - RNA, 23 nt, Homo sapiens
  source 1..23: mol_type = genomic RNA
cggcgtccgcaagtgtaagaagt

SEQ ID NO: 349 - RNA, 23 nt, Homo sapiens
  source 1..23: mol_type = genomic RNA
ggcgtccgcaagtgtaagaagtg

SEQ ID NO: 350 - RNA, 23 nt, Homo sapiens
  source 1..23: mol_type = genomic RNA
gcgtccgcaagtgtaagaagtgc

SEQ ID NO: 351 - RNA, 23 nt, Homo sapiens
  source 1..23: mol_type = genomic RNA
cgtccgcaagtgtaagaagtgcg

SEQ ID NO: 352 - RNA, 23 nt, Homo sapiens
  source 1..23: mol_type = genomic RNA
gtccgcaagtgtaagaagtgcga

SEQ ID NO: 353 - RNA, 23 nt, Homo sapiens
  source 1..23: mol_type = genomic RNA
tccgcaagtgtaagaagtgcgaa

SEQ ID NO: 354 - RNA, 23 nt, Homo sapiens
  source 1..23: mol_type = genomic RNA
ccgcaagtgtaagaagtgcgaag

SEQ ID NO: 355 - RNA, 23 nt, Homo sapiens
  source 1..23: mol_type = genomic RNA
gcaagtgtaagaagtgcgaaggg

SEQ ID NO: 356 - RNA, 23 nt, Homo sapiens
  source 1..23: mol_type = genomic RNA
caagtgtaagaagtgcgaagggc

SEQ ID NO: 357 - RNA, 23 nt, Homo sapiens
  source 1..23: mol_type = genomic RNA
aagtgtaagaagtgcgaagggcc

SEQ ID NO: 358 - RNA, 23 nt, Homo sapiens
  source 1..23: mol_type = genomic RNA
agtgtaagaagtgcgaagggcct

SEQ ID NO: 359 - RNA, 23 nt, Homo sapiens
  source 1..23: mol_type = genomic RNA
gtgtaagaagtgcgaagggcctt

SEQ ID NO: 360 - RNA, 23 nt, Homo sapiens
  source 1..23: mol_type = genomic RNA
tgtaagaagtgcgaagggccttg

SEQ ID NO: 361 - RNA, 23 nt, Homo sapiens
  source 1..23: mol_type = genomic RNA
gtaagaagtgcgaagggccttgc

SEQ ID NO: 362 - RNA, 23 nt, Homo sapiens
  source 1..23: mol_type = genomic RNA
taagaagtgcgaagggccttgcc

SEQ ID NO: 363 - RNA, 23 nt, Homo sapiens
  source 1..23: mol_type = genomic RNA
gaagtgcgaagggccttgccgca

SEQ ID NO: 364 - RNA, 23 nt, Homo sapiens
  source 1..23: mol_type = genomic RNA
aagtgcgaagggccttgccgcaa

SEQ ID NO: 365 - RNA, 23 nt, Homo sapiens
  source 1..23: mol_type = genomic RNA
agtgcgaagggccttgccgcaaa

SEQ ID NO: 366 - RNA, 23 nt, Homo sapiens
  source 1..23: mol_type = genomic RNA
gtgcgaagggccttgccgcaaag

SEQ ID NO: 367 - RNA, 23 nt, Homo sapiens
  source 1..23: mol_type = genomic RNA
tgcgaagggccttgccgcaaagt

SEQ ID NO: 368 - RNA, 23 nt, Homo sapiens
  source 1..23: mol_type = genomic RNA
cgaagggccttgccgcaaagtgt

SEQ ID NO: 369 - RNA, 23 nt, Homo sapiens
  source 1..23: mol_type = genomic RNA
gaagggccttgccgcaaagtgtg

SEQ ID NO: 370 - RNA, 23 nt, Homo sapiens
  source 1..23: mol_type = genomic RNA
aagggccttgccgcaaagtgtgt

SEQ ID NO: 371 - RNA, 23 nt, Homo sapiens
  source 1..23: mol_type = genomic RNA
agggccttgccgcaaagtgtgta

SEQ ID NO: 372 - RNA, 23 nt, Homo sapiens
  source 1..23: mol_type = genomic RNA
gggccttgccgcaaagtgtgtaa

SEQ ID NO: 373 - RNA, 23 nt, Homo sapiens
  source 1..23: mol_type = genomic RNA
gccttgccgcaaagtgtgtaacg

SEQ ID NO: 374 - RNA, 23 nt, Homo sapiens
  source 1..23: mol_type = genomic RNA
acggaataggtattggtgaattt

SEQ ID NO: 375 - RNA, 23 nt, Homo sapiens
  source 1..23: mol_type = genomic RNA
cggaataggtattggtgaattta

SEQ ID NO: 376 - RNA, 23 nt, Homo sapiens
  source 1..23: mol_type = genomic RNA
ggaataggtattggtgaatttaa

SEQ ID NO: 377 - RNA, 23 nt, Homo sapiens
  source 1..23: mol_type = genomic RNA
gaataggtattggtgaatttaaa

SEQ ID NO: 378 - RNA, 23 nt, Homo sapiens
  source 1..23: mol_type = genomic RNA
ataggtattggtgaatttaaaga

SEQ ID NO: 379 - RNA, 23 nt, Homo sapiens
  source 1..23: mol_type = genomic RNA
taggtattggtgaatttaaagac

SEQ ID NO: 380 - RNA, 23 nt, Homo sapiens
  source 1..23: mol_type = genomic RNA
ctcactctccataaatgctacga

SEQ ID NO: 381 - RNA, 23 nt, Homo sapiens
  source 1..23: mol_type = genomic RNA
tattaaacacttcaaaaactgca

SEQ ID NO: 382 - RNA, 23 nt, Homo sapiens
  source 1..23: mol_type = genomic RNA
cacttcaaaaactgcacctccat

SEQ ID NO: 383 - RNA, 23 nt, Homo sapiens
  source 1..23: mol_type = genomic RNA
acttcaaaaactgcacctccatc

SEQ ID NO: 384 - RNA, 23 nt, Homo sapiens
  source 1..23: mol_type = genomic RNA
cttcaaaaactgcacctccatca

SEQ ID NO: 385 - RNA, 23 nt, Homo sapiens
  source 1..23: mol_type = genomic RNA
ttcaaaaactgcacctccatcag

SEQ ID NO: 386 - RNA, 23 nt, Homo sapiens
  source 1..23: mol_type = genomic RNA
tcaaaaactgcacctccatcagt

SEQ ID NO: 387 - RNA, 23 nt, Homo sapiens
  source 1..23: mol_type = genomic RNA
aaactgcacctccatcagtggcg

SEQ ID NO: 388 - RNA, 23 nt, Homo sapiens
  source 1..23: mol_type = genomic RNA
tgcacctccatcagtggcgatct

SEQ ID NO: 389 - RNA, 23 nt, Homo sapiens
  source 1..23: mol_type = genomic RNA
gcacctccatcagtggcgatctc

SEQ ID NO: 390 - RNA, 23 nt, Homo sapiens
  source 1..23: mol_type = genomic RNA
acctccatcagtggcgatctcca

SEQ ID NO: 391 - RNA, 23 nt, Homo sapiens
  source 1..23: mol_type = genomic RNA
tccatcagtggcgatctccacat

SEQ ID NO: 392 - RNA, 23 nt, Homo sapiens
  source 1..23: mol_type = genomic RNA
agtggcgatctccacatcctgcc

SEQ ID NO: 393 - RNA, 23 nt, Homo sapiens
  source 1..23: mol_type = genomic RNA
gtggcgatctccacatcctgccg

SEQ ID NO: 394 - RNA, 23 nt, Homo sapiens
  source 1..23: mol_type = genomic RNA
tggcgatctccacatcctgccgg

SEQ ID NO: 395 - RNA, 23 nt, Homo sapiens
  source 1..23: mol_type = genomic RNA
ggcgatctccacatcctgccggt

SEQ ID NO: 396 - RNA, 23 nt, Homo sapiens
  source 1..23: mol_type = genomic RNA
gcgatctccacatcctgccggtg

SEQ ID NO: 397 - RNA, 23 nt, Homo sapiens
  source 1..23: mol_type = genomic RNA
ctccacatcctgccggtggcatt

SEQ ID NO: 398 - RNA, 23 nt, Homo sapiens
  source 1..23: mol_type = genomic RNA
tccacatcctgccggtggcattt

SEQ ID NO: 399 - RNA, 23 nt, Homo sapiens
  source 1..23: mol_type = genomic RNA
ccacatcctgccggtggcattta

SEQ ID NO: 400 - RNA, 23 nt, Homo sapiens
  source 1..23: mol_type = genomic RNA
acatcctgccggtggcatttagg

SEQ ID NO: 401 - RNA, 23 nt, Homo sapiens
  source 1..23: mol_type = genomic RNA
tcctgccggtggcatttaggggt

SEQ ID NO: 402 - RNA, 23 nt, Homo sapiens
  source 1..23: mol_type = genomic RNA
cctgccggtggcatttaggggtg

SEQ ID NO: 403 - RNA, 23 nt, Homo sapiens
  source 1..23: mol_type = genomic RNA
ctgccggtggcatttaggggtga

SEQ ID NO: 404 - RNA, 23 nt, Homo sapiens
  source 1..23: mol_type = genomic RNA
tgccggtggcatttaggggtgac

SEQ ID NO: 405 - RNA, 23 nt, Homo sapiens
  source 1..23: mol_type = genomic RNA
cggtggcatttaggggtgactcc

SEQ ID NO: 406 - RNA, 23 nt, Homo sapiens
  source 1..23: mol_type = genomic RNA
tggcatttaggggtgactccttc

SEQ ID NO: 407 - RNA, 23 nt, Homo sapiens
  source 1..23: mol_type = genomic RNA
ggcatttaggggtgactccttca

SEQ ID NO: 408 - RNA, 23 nt, Homo sapiens
  source 1..23: mol_type = genomic RNA
gcatttaggggtgactccttcac

SEQ ID NO: 409 - RNA, 23 nt, Homo sapiens
  source 1..23: mol_type = genomic RNA
catttaggggtgactccttcaca

SEQ ID NO: 410 - RNA, 23 nt, Homo sapiens
  source 1..23: mol_type = genomic RNA
atttaggggtgactccttcacac

SEQ ID NO: 411 - RNA, 23 nt, Homo sapiens
  source 1..23: mol_type = genomic RNA
tttaggggtgactccttcacaca

SEQ ID NO: 412 - RNA, 23 nt, Homo sapiens
  source 1..23: mol_type = genomic RNA
cctctggatccacaggaactgga

SEQ ID NO: 413 - RNA, 23 nt, Homo sapiens
  source 1..23: mol_type = genomic RNA
tggatccacaggaactggatatt

SEQ ID NO: 414 - RNA, 23 nt, Homo sapiens
  source 1..23: mol_type = genomic RNA
ggatccacaggaactggatattc

SEQ ID NO: 415 - RNA, 23 nt, Homo sapiens
  source 1..23: mol_type = genomic RNA
atccacaggaactggatattctg

SEQ ID NO: 416 - RNA, 23 nt, Homo sapiens
  source 1..23: mol_type = genomic RNA
tccacaggaactggatattctga

SEQ ID NO: 417 - RNA, 23 nt, Homo sapiens
  source 1..23: mol_type = genomic RNA
gaactggatattctgaaaaccgt

SEQ ID NO: 418 - RNA, 23 nt, Homo sapiens
  source 1..23: mol_type = genomic RNA
atattctgaaaaccgtaaaggaa

SEQ ID NO: 419 - RNA, 23 nt, Homo sapiens
  source 1..23: mol_type = genomic RNA
tattctgaaaaccgtaaaggaaa

SEQ ID NO: 420 - RNA, 23 nt, Homo sapiens
  source 1..23: mol_type = genomic RNA
tctgaaaaccgtaaaggaaatca

SEQ ID NO: 421 - RNA, 23 nt, Homo sapiens
  source 1..23: mol_type = genomic RNA
ctgaaaaccgtaaaggaaatcac

SEQ ID NO: 422 - DNA, 21 bp, synthetic construct
  source 1..21: mol_type = other DNA
  misc_feature 1..19: note = RNA
  misc_feature 20..21: note = DNA
cggccggagtcccgagctatt

SEQ ID NO: 423 - DNA, 21 bp, synthetic construct
  source 1..21: mol_type = other DNA
  misc_feature 1..19: note = RNA
  misc_feature 20..21: note = DNA
tagctcgggactccggccgtt

SEQ ID NO: 424 - DNA, 21 bp, synthetic construct
  source 1..21: mol_type = other DNA
  misc_feature 1..19: note = RNA
  misc_feature 20..21: note = DNA
ccggagtcccgagctagcctt

SEQ ID NO: 425 - DNA, 21 bp, synthetic construct
  source 1..21: mol_type = other DNA
  misc_feature 1..19: note = RNA
  misc_feature 20..21: note = DNA
ggctagctcgggactccggtt

SEQ ID NO: 426 - DNA, 21 bp, synthetic construct
  source 1..21: mol_type = other DNA
  misc_feature 1..19: note = RNA
  misc_feature 20..21: note = DNA
cggagtcccgagctagccctt

SEQ ID NO: 427 - DNA, 21 bp, synthetic construct
  source 1..21: mol_type = other DNA
  misc_feature 1..19: note = RNA
  misc_feature 20..21: note = DNA
gggctagctcgggactccgtt

SEQ ID NO: 428 - DNA, 21 bp, synthetic construct
  source 1..21: mol_type = other DNA
  misc_feature 1..19: note = RNA
  misc_feature 20..21: note = DNA
ggagtcccgagctagcccctt

SEQ ID NO: 429 - DNA, 21 bp, synthetic construct
  source 1..21: mol_type = other DNA
  misc_feature 1..19: note = RNA
  misc_feature 20..21: note = DNA
ggggctagctcgggactcctt

SEQ ID NO: 430 - DNA, 21 bp, synthetic construct
  source 1..21: mol_type = other DNA
  misc_feature 1..19: note = RNA
  misc_feature 20..21: note = DNA
gagtcccgagctagccccgtt

SEQ ID NO: 431 - DNA, 21 bp, synthetic construct
  source 1..21: mol_type = other DNA
  misc_feature 1..19: note = RNA
  misc_feature 20..21: note = DNA
cggggctagctcgggactctt

SEQ ID NO: 432 - DNA, 21 bp, synthetic construct
  source 1..21: mol_type = other DNA
  misc_feature 1..19: note = RNA
  misc_feature 20..21: note = DNA
agtcccgagctagccccggtt

SEQ ID NO: 433 - DNA, 21 bp, synthetic construct
  source 1..21: mol_type = other DNA
  misc_feature 1..19: note = RNA
  misc_feature 20..21: note = DNA
ccggggctagctcgggacttt

SEQ ID NO: 434 - DNA, 21 bp, synthetic construct
  source 1..21: mol_type = other DNA
  misc_feature 1..19: note = RNA
  misc_feature 20..21: note = DNA
gtcccgagctagccccggctt

SEQ ID NO: 435 - DNA, 21 bp, synthetic construct
  source 1..21: mol_type = other DNA
  misc_feature 1..19: note = RNA
  misc_feature 20..21: note = DNA
gccggggctagctcgggactt

SEQ ID NO: 436 - DNA, 21 bp, synthetic construct
  source 1..21: mol_type = other DNA
  misc_feature 1..19: note = RNA
  misc_feature 20..21: note = DNA
cccgagctagccccggcggtt

SEQ ID NO: 437 - DNA, 21 bp, synthetic construct
  source 1..21: mol_type = other DNA
  misc_feature 1..19: note = RNA
  misc_feature 20..21: note = DNA
ccgccggggctagctcgggtt

SEQ ID NO: 438 - DNA, 21 bp, synthetic construct
  source 1..21: mol_type = other DNA
  misc_feature 1..19: note = RNA
  misc_feature 20..21: note = DNA
ggacgacaggccacctcgttt

SEQ ID NO: 439 - DNA, 21 bp, synthetic construct
  source 1..21: mol_type = other DNA
  misc_feature 1..19: note = RNA
  misc_feature 20..21: note = DNA
acgaggtggcctgtcgtcctt

SEQ ID NO: 440 - DNA, 21 bp, synthetic construct
  source 1..21: mol_type = other DNA
  misc_feature 1..19: note = RNA
  misc_feature 20..21: note = DNA
gacgacaggccacctcgtctt

SEQ ID NO: 441 - DNA, 21 bp, synthetic construct
  source 1..21: mol_type = other DNA
  misc_feature 1..19: note = RNA
  misc_feature 20..21: note = DNA
gacgaggtggcctgtcgtctt

SEQ ID NO: 442 - DNA, 21 bp, synthetic construct
  source 1..21: mol_type = other DNA
  misc_feature 1..19: note = RNA
  misc_feature 20..21: note = DNA
acgacaggccacctcgtcgtt

SEQ ID NO: 443 - DNA, 21 bp, synthetic construct
  source 1..21: mol_type = other DNA
  misc_feature 1..19: note = RNA
  misc_feature 20..21: note = DNA
cgacgaggtggcctgtcgttt

SEQ ID NO: 444 - DNA, 21 bp, synthetic construct
  source 1..21: mol_type = other DNA
  misc_feature 1..19: note = RNA
  misc_feature 20..21: note = DNA
cgacaggccacctcgtcggtt

SEQ ID NO: 445 - DNA, 21 bp, synthetic construct
  source 1..21: mol_type = other DNA
  misc_feature 1..19: note = RNA
  misc_feature 20..21: note = DNA
ccgacgaggtggcctgtcgtt

SEQ ID NO: 446 - DNA, 21 bp, synthetic construct
  source 1..21: mol_type = other DNA
  misc_feature 1..19: note = RNA
  misc_feature 20..21: note = DNA
gacaggccacctcgtcggctt

SEQ ID NO: 447 - DNA, 21 bp, synthetic construct
  source 1..21: mol_type = other DNA
  misc_feature 1..19: note = RNA
  misc_feature 20..21: note = DNA
gccgacgaggtggcctgtctt

SEQ ID NO: 448 - DNA, 21 bp, synthetic construct
  source 1..21: mol_type = other DNA
  misc_feature 1..19: note = RNA
  misc_feature 20..21: note = DNA
caggccacctcgtcggcgttt

SEQ ID NO: 449 - DNA, 21 bp, synthetic construct
  source 1..21: mol_type = other DNA
  misc_feature 1..19: note = RNA
  misc_feature 20..21: note = DNA
acgccgacgaggtggcctgtt

SEQ ID NO: 450 - DNA, 21 bp, synthetic construct
  source 1..21: mol_type = other DNA
  misc_feature 1..19: note = RNA
  misc_feature 20..21: note = DNA
aggccacctcgtcggcgtctt

SEQ ID NO: 451 - DNA, 21 bp, synthetic construct
  source 1..21: mol_type = other DNA
  misc_feature 1..19: note = RNA
  misc_feature 20..21: note = DNA
gacgccgacgaggtggccttt

SEQ ID NO: 452 - DNA, 21 bp, synthetic construct
  source 1..21: mol_type = other DNA
  misc_feature 1..19: note = RNA
  misc_feature 20..21: note = DNA
ggccacctcgtcggcgtcctt

SEQ ID NO: 453 - DNA, 21 bp, synthetic construct
  source 1..21: mol_type = other DNA
  misc_feature 1..19: note = RNA
  misc_feature 20..21: note = DNA
ggacgccgacgaggtggcctt

SEQ ID NO: 454 - DNA, 21 bp, synthetic construct
  source 1..21: mol_type = other DNA
  misc_feature 1..19: note = RNA
  misc_feature 20..21: note = DNA
gccacctcgtcggcgtccgtt

SEQ ID NO: 455 - DNA, 21 bp, synthetic construct
  source 1..21: mol_type = other DNA
  misc_feature 1..19: note = RNA
  misc_feature 20..21: note = DNA
cggacgccgacgaggtggctt

SEQ ID NO: 456 - DNA, 21 bp, synthetic construct
  source 1..21: mol_type = other DNA
  misc_feature 1..19: note = RNA
  misc_feature 20..21: note = DNA
ccacctcgtcggcgtccgctt

SEQ ID NO: 457 - DNA, 21 bp, synthetic construct
  source 1..21: mol_type = other DNA
  misc_feature 1..19: note = RNA
  misc_feature 20..21: note = DNA
gcggacgccgacgaggtggtt

SEQ ID NO: 458 - DNA, 21 bp, synthetic construct
  source 1..21: mol_type = other DNA
  misc_feature 1..19: note = RNA
  misc_feature 20..21: note = DNA
cacctcgtcggcgtccgcctt

SEQ ID NO: 459 - DNA, 21 bp, synthetic construct
  source 1..21: mol_type = other DNA
  misc_feature 1..19: note = RNA
  misc_feature 20..21: note = DNA
ggcggacgccgacgaggtgtt

SEQ ID NO: 460 - DNA, 21 bp, synthetic construct
  source 1..21: mol_type = other DNA
  misc_feature 1..19: note = RNA
  misc_feature 20..21: note = DNA
acctcgtcggcgtccgccctt

SEQ ID NO: 461 - DNA, 21 bp, synthetic construct
  source 1..21: mol_type = other DNA
  misc_feature 1..19: note = RNA
  misc_feature 20..21: note = DNA
gggcggacgccgacgaggttt

SEQ ID NO: 462 - DNA, 21 bp, synthetic construct
  source 1..21: mol_type = other DNA
  misc_feature 1..19: note = RNA
  misc_feature 20..21: note = DNA
cctcgtcggcgtccgcccgtt

SEQ ID NO: 463 - DNA, 21 bp, synthetic construct
  source 1..21: mol_type = other DNA
cgggcggacgccgacgaggtt

SEQ ID NO: 464 - DNA, 21 bp, synthetic construct
  source 1..21: mol_type = other DNA
  misc_feature 1..19: note = RNA
  misc_feature 20..21: note = DNA
ctcgtcggcgtccgcccgatt

SEQ ID NO: 465 - DNA, 21 bp, synthetic construct
  source 1..21: mol_type = other DNA
  misc_feature 1..19: note = RNA
  misc_feature 20..21: note = DNA
tcgggcggacgccgacgagtt

SEQ ID NO: 466 - DNA, 21 bp, synthetic construct
  source 1..21: mol_type = other DNA
  misc_feature 1..19: note = RNA
  misc_feature 20..21: note = DNA
tcgtcggcgtccgcccgagtt

SEQ ID NO: 467 - DNA, 21 bp, synthetic construct
  source 1..21: mol_type = other DNA
  misc_feature 1..19: note = RNA
  misc_feature 20..21: note = DNA
ctcgggcggacgccgacgatt

SEQ ID NO: 468 - DNA, 21 bp, synthetic construct
  source 1..21: mol_type = other DNA
  misc_feature 1..19: note = RNA
  misc_feature 20..21: note = DNA
cgtcggcgtccgcccgagttt

SEQ ID NO: 469 - DNA, 21 bp, synthetic construct
  source 1..21: mol_type = other DNA
  misc_feature 1..19: note = RNA
  misc_feature 20..21: note = DNA
actcgggcggacgccgacgtt

SEQ ID NO: 470 - DNA, 21 bp, synthetic construct
  source 1..21: mol_type = other DNA
  misc_feature 1..19: note = RNA
  misc_feature 20..21: note = DNA
gtcggcgtccgcccgagtctt

SEQ ID NO: 471 - DNA, 21 bp, synthetic construct
  source 1..21: mol_type = other DNA
  misc_feature 1..19: note = RNA
  misc_feature 20..21: note = DNA
gactcgggcggacgccgactt

SEQ ID NO: 472 - DNA, 21 bp, synthetic construct
  source 1..21: mol_type = other DNA
  misc_feature 1..19: note = RNA
  misc_feature 20..21: note = DNA
tcggcgtccgcccgagtcctt

SEQ ID NO: 473 - DNA, 21 bp, synthetic construct
  source 1..21: mol_type = other DNA
  misc_feature 1..19: note = RNA
  misc_feature 20..21: note = DNA
ggactcgggcggacgccgatt

SEQ ID NO: 474 - DNA, 21 bp, synthetic construct
  source 1..21: mol_type = other DNA
  misc_feature 1..19: note = RNA
  misc_feature 20..21: note = DNA
gcgtccgcccgagtccccgtt

SEQ ID NO: 475 - DNA, 21 bp, synthetic construct
  source 1..21: mol_type = other DNA
  misc_feature 1..19: note = RNA
  misc_feature 20..21: note = DNA
cggggactcgggcggacgctt

SEQ ID NO: 476 - DNA, 21 bp, synthetic construct
  source 1..21: mol_type = other DNA
  misc_feature 1..19: note = RNA
  misc_feature 20..21: note = DNA
cgtccgcccgagtccccgctt

SEQ ID NO: 477 - DNA, 21 bp, synthetic construct
  source 1..21: mol_type = other DNA
  misc_feature 1..19: note = RNA
  misc_feature 20..21: note = DNA
gcggggactcgggcggacgtt

SEQ ID NO: 478 - DNA, 21 bp, synthetic construct
  source 1..21: mol_type = other DNA
  misc_feature 1..19: note = RNA
  misc_feature 20..21: note = DNA
gcccgagtccccgcctcgctt

SEQ ID NO: 479 - DNA, 21 bp, synthetic construct
  source 1..21: mol_type = other DNA
  misc_feature 1..19: note = RNA
  misc_feature 20..21: note = DNA
gcgaggcggggactcgggctt

SEQ ID NO: 480 - DNA, 21 bp, synthetic construct
  source 1..21: mol_type = other DNA
aacgccacaaccaccgcgctt

SEQ ID NO: 481 - DNA, 21 bp, synthetic construct
  source 1..21: mol_type = other DNA
  misc_feature 1..19: note = RNA
  misc_feature 20..21: note = DNA
gcgcggtggttgtggcgtttt

SEQ ID NO: 482 - DNA, 21 bp, synthetic construct
  source 1..21: mol_type = other DNA
acgccacaaccaccgcgcatt

SEQ ID NO: 483 - DNA, 21 bp, synthetic construct
  source 1..21: mol_type = other DNA
  misc_feature 1..19: note = RNA
  misc_feature 20..21: note = DNA
tgcgcggtggttgtggcgttt

SEQ ID NO: 484 - DNA, 21 bp, synthetic construct
  source 1..21: mol_type = other DNA
cgccacaaccaccgcgcactt

SEQ ID NO: 485 - DNA, 21 bp, synthetic construct
  source 1..21: mol_type = other DNA
  misc_feature 1..19: note = RNA
  misc_feature 20..21: note = DNA
gtgcgcggtggttgtggcgtt

SEQ ID NO: 486 - DNA, 21 bp, synthetic construct
  source 1..21: mol_type = other DNA
ccacaaccaccgcgcacggtt

SEQ ID NO: 487 - DNA, 21 bp, synthetic construct
  source 1..21: mol_type = other DNA
  misc_feature 1..19: note = RNA
  misc_feature 20..21: note = DNA
ccgtgcgcggtggttgtggtt

SEQ ID NO: 488 - DNA, 21 bp, synthetic construct
  source 1..21: mol_type = other DNA
cacaaccaccgcgcacggctt

SEQ ID NO: 489 - DNA, 21 bp, synthetic construct
  source 1..21: mol_type = other DNA
  misc_feature 1..19: note = RNA
  misc_feature 20..21: note = DNA
gccgtgcgcggtggttgtgtt

SEQ ID NO: 490 - DNA, 21 bp, synthetic construct
  source 1..21: mol_type = other DNA
acaaccaccgcgcacggcctt

SEQ ID NO: 491 - DNA, 21 bp, synthetic construct
  source 1..21: mol_type = other DNA
  misc_feature 1..19: note = RNA
  misc_feature 20..21: note = DNA
ggccgtgcgcggtggttgttt

SEQ ID NO: 492 - DNA, 21 bp, synthetic construct
  source 1..21: mol_type = other DNA
  misc_feature 1..19: note = RNA
  misc_feature 20..21: note = DNA
atgcgaccctccgggacggtt

SEQ ID NO: 493 - DNA, 21 bp, synthetic construct
  source 1..21: mol_type = other DNA
  misc_feature 1..19: note = RNA
  misc_feature 20..21: note = DNA
ccgtcccggagggtcgcattt

SEQ ID NO: 494 - DNA, 21 bp, synthetic construct
  source 1..21: mol_type = other DNA
  misc_feature 1..19: note = RNA
  misc_feature 20..21: note = DNA
tgcgaccctccgggacggctt

SEQ ID NO: 495 - DNA, 21 bp, synthetic construct
  source 1..21: mol_type = other DNA
  misc_feature 1..19: note = RNA
  misc_feature 20..21: note = DNA
gccgtcccggagggtcgcatt

SEQ ID NO: 496 - DNA, 21 bp, synthetic construct
  source 1..21: mol_type = other DNA
  misc_feature 1..19: note = RNA
  misc_feature 20..21: note = DNA
gcgaccctccgggacggcctt

SEQ ID NO: 497 - DNA, 21 bp, synthetic construct
  source 1..21: mol_type = other DNA
  misc_feature 1..19: note = RNA
  misc_feature 20..21: note = DNA
ggccgtcccggagggtcgctt

SEQ ID NO: 498 - DNA, 21 bp, synthetic construct
  source 1..21: mol_type = other DNA
  misc_feature 1..19: note = RNA
  misc_feature 20..21: note = DNA
gaccctccgggacggccggtt

SEQ ID NO: 499 - DNA, 21 bp, synthetic construct
  source 1..21: mol_type = other DNA
  misc_feature 1..19: note = RNA
  misc_feature 20..21: note = DNA
ccggccgtcccggagggtctt

SEQ ID NO: 500 - DNA, 21 bp, synthetic construct
  source 1..21: mol_type = other DNA
  misc_feature 1..19: note = RNA
  misc_feature 20..21: note = DNA
accctccgggacggccgggtt

SEQ ID NO: 501 - DNA, 21 bp, synthetic construct
  source 1..21: mol_type = other DNA
  misc_feature 1..19: note = RNA
  misc_feature 20..21: note = DNA
cccggccgtcccggagggttt

SEQ ID NO: 502 - DNA, 21 bp, synthetic construct
  source 1..21: mol_type = other DNA
  misc_feature 1..19: note = RNA
  misc_feature 20..21: note = DNA
cctccgggacggccggggctt

SEQ ID NO: 503 - DNA, 21 bp, synthetic construct
  source 1..21: mol_type = other DNA
  misc_feature 1..19: note = RNA
  misc_feature 20..21: note = DNA
gccccggccgtcccggaggtt

SEQ ID NO: 504 - DNA, 21 bp, synthetic construct
  source 1..21: mol_type = other DNA
  misc_feature 1..19: note = RNA
  misc_feature 20..21: note = DNA
agaaagtttgccaaggcactt

SEQ ID NO: 505 - DNA, 21 bp, synthetic construct
  source 1..21: mol_type = other DNA
  misc_feature 1..19: note = RNA
  misc_feature 20..21: note = DNA
gtgccttggcaaactttcttt

SEQ ID NO: 506 - DNA, 21 bp, synthetic construct
  source 1..21: mol_type = other DNA
  misc_feature 1..19: note = RNA
  misc_feature 20..21: note = DNA
gaaagtttgccaaggcacgtt

SEQ ID NO: 507 - DNA, 21 bp, synthetic construct
  source 1..21: mol_type = other DNA
  misc_feature 1..19: note = RNA
  misc_feature 20..21: note = DNA
cgtgccttggcaaactttctt

SEQ ID NO: 508 - DNA, 21 bp, synthetic construct
  source 1..21: mol_type = other DNA
  misc_feature 1..19: note = RNA
  misc_feature 20..21: note = DNA
aagtttgccaaggcacgagtt

SEQ ID NO: 509 - DNA, 21 bp, synthetic construct
  source 1..21: mol_type = other DNA
  misc_feature 1..19: note = RNA
  misc_feature 20..21: note = DNA
ctcgtgccttggcaaactttt

SEQ ID NO: 510 - DNA, 21 bp, synthetic construct
  source 1..21: mol_type = other DNA
  misc_feature 1..19: note = RNA
  misc_feature 20..21: note = DNA
agtttgccaaggcacgagttt

SEQ ID NO: 511 - DNA, 21 bp, synthetic construct
  source 1..21: mol_type = other DNA
  misc_feature 1..19: note = RNA
  misc_feature 20..21: note = DNA
actcgtgccttggcaaacttt

SEQ ID NO: 512 - DNA, 21 bp, synthetic construct
  source 1..21: mol_type = other DNA
  misc_feature 1..19: note = RNA
  misc_feature 20..21: note = DNA
gtttgccaaggcacgagtatt

SEQ ID NO: 513 - DNA, 21 bp, synthetic construct
  source 1..21: mol_type = other DNA
  misc_feature 1..19: note = RNA
  misc_feature 20..21: note = DNA
tactcgtgccttggcaaactt

SEQ ID NO: 514 - DNA, 21 bp, synthetic construct
  source 1..21: mol_type = other DNA
  misc_feature 1..19: note = RNA
  misc_feature 20..21: note = DNA
tttgccaaggcacgagtaatt

SEQ ID NO: 515 - DNA, 21 bp, synthetic construct
  source 1..21: mol_type = other DNA
  misc_feature 1..19: note = RNA
  misc_feature 20..21: note = DNA
ttactcgtgccttggcaaatt

SEQ ID NO: 516 - DNA, 21 bp, synthetic construct
  source 1..21: mol_type = other DNA
  misc_feature 1..19: note = RNA
  misc_feature 20..21: note = DNA
ttgccaaggcacgagtaactt

SEQ ID NO: 517 - DNA, 21 bp, synthetic construct
  source 1..21: mol_type = other DNA
  misc_feature 1..19: note = RNA
  misc_feature 20..21: note = DNA
gttactcgtgccttggcaatt

SEQ ID NO: 518 - DNA, 21 bp, synthetic construct
  source 1..21: mol_type = other DNA
  misc_feature 1..19: note = RNA
  misc_feature 20..21: note = DNA
tgccaaggcacgagtaacatt

SEQ ID NO: 519 - DNA, 21 bp, synthetic construct
  source 1..21: mol_type = other DNA
  misc_feature 1..19: note = RNA
  misc_feature 20..21: note = DNA
tgttactcgtgccttggcatt

SEQ ID NO: 520 - DNA, 21 bp, synthetic construct
  source 1..21: mol_type = other DNA
  misc_feature 1..19: note = RNA
  misc_feature 20..21: note = DNA
gccaaggcacgagtaacaatt

SEQ ID NO: 521 - DNA, 21 bp, synthetic construct
  source 1..21: mol_type = other DNA
  misc_feature 1..19: note = RNA
  misc_feature 20..21: note = DNA
ttgttactcgtgccttggctt

SEQ ID NO: 522 - DNA, 21 bp, synthetic construct
  source 1..21: mol_type = other DNA
  misc_feature 1..19: note = RNA
  misc_feature 20..21: note = DNA
acgcagttgggcacttttgtt

SEQ ID NO: 523 - DNA, 21 bp, synthetic construct
  source 1..21: mol_type = other DNA
  misc_feature 1..19: note = RNA
  misc_feature 20..21: note = DNA
caaaagtgcccaactgcgttt

SEQ ID NO: 524 - DNA, 21 bp, synthetic construct
  source 1..21: mol_type = other DNA
  misc_feature 1..19: note = RNA
  misc_feature 20..21: note = DNA
cgcagttgggcacttttgatt

SEQ ID NO: 525 - DNA, 21 bp, synthetic construct
  source 1..21: mol_type = other DNA
  misc_feature 1..19: note = RNA
  misc_feature 20..21: note = DNA
tcaaaagtgcccaactgcgtt

SEQ ID NO: 526 - DNA, 21 bp, synthetic construct
  source 1..21: mol_type = other DNA
  misc_feature 1..19: note = RNA
  misc_feature 20..21: note = DNA
gcagttgggcacttttgaatt

SEQ ID NO: 527 - DNA, 21 bp, synthetic construct
  source 1..21: mol_type = other DNA
  misc_feature 1..19: note = RNA
  misc_feature 20..21: note = DNA
ttcaaaagtgcccaactgctt

SEQ ID NO: 528 - DNA, 21 bp, synthetic construct
  source 1..21: mol_type = other DNA
  misc_feature 1..19: note = RNA
  misc_feature 20..21: note = DNA
cagttgggcacttttgaagtt

SEQ ID NO: 529 - DNA, 21 bp, synthetic construct
  source 1..21: mol_type = other DNA
  misc_feature 1..19: note = RNA
  misc_feature 20..21: note = DNA
cttcaaaagtgcccaactgtt

SEQ ID NO: 530 - DNA, 21 bp, synthetic construct
  source 1..21: mol_type = other DNA
  misc_feature 1..19: note = RNA
  misc_feature 20..21: note = DNA
agttgggcacttttgaagatt

SEQ ID NO: 531 - DNA, 21 bp, synthetic construct
  source 1..21: mol_type = other DNA
  misc_feature 1..19: note = RNA
  misc_feature 20..21: note = DNA
tcttcaaaagtgcccaacttt

SEQ ID NO: 532 - DNA, 21 bp, synthetic construct
  source 1..21: mol_type = other DNA
  misc_feature 1..19: note = RNA
  misc_feature 20..21: note = DNA
gttgggcacttttgaagattt

SEQ ID NO: 533 - DNA, 21 bp, synthetic construct
  source 1..21: mol_type = other DNA
  misc_feature 1..19: note = RNA
  misc_feature 20..21: note = DNA
atcttcaaaagtgcccaactt

SEQ ID NO: 534 - DNA, 21 bp, synthetic construct
  source 1..21: mol_type = other DNA
  misc_feature 1..19: note = RNA
  misc_feature 20..21: note = DNA
ttgggcacttttgaagatctt

SEQ ID NO: 535 - DNA, 21 bp, synthetic construct
  source 1..21: mol_type = other DNA
  misc_feature 1..19: note = RNA
  misc_feature 20..21: note = DNA
gatcttcaaaagtgcccaatt

SEQ ID NO: 536 - DNA, 21 bp, synthetic construct
  source 1..21: mol_type = other DNA
  misc_feature 1..19: note = RNA
  misc_feature 20..21: note = DNA
tgggcacttttgaagatcatt

SEQ ID NO: 537 - DNA, 21 bp, synthetic construct
  source 1..21: mol_type = other DNA
  misc_feature 1..19: note = RNA
  misc_feature 20..21: note = DNA
tgatcttcaaaagtgcccatt

SEQ ID NO: 538 - DNA, 21 bp, synthetic construct
  source 1..21: mol_type = other DNA
  misc_feature 1..19: note = RNA
  misc_feature 20..21: note = DNA
gggcacttttgaagatcattt

SEQ ID NO: 539 - DNA, 21 bp, synthetic construct
  source 1..21: mol_type = other DNA
  misc_feature 1..19: note = RNA
  misc_feature 20..21: note = DNA
atgatcttcaaaagtgccctt

SEQ ID NO: 540 - DNA, 21 bp, synthetic construct
  source 1..21: mol_type = other DNA
  misc_feature 1..19: note = RNA
  misc_feature 20..21: note = DNA
ttgaagatcattttctcagtt

SEQ ID NO: 541 - DNA, 21 bp, synthetic construct
  source 1..21: mol_type = other DNA
  misc_feature 1..19: note = RNA
  misc_feature 20..21: note = DNA
ctgagaaaatgatcttcaatt

SEQ ID NO: 542 - DNA, 21 bp, synthetic construct
  source 1..21: mol_type = other DNA
  misc_feature 1..19: note = RNA
  misc_feature 20..21: note = DNA
gaagatcattttctcagcctt

SEQ ID NO: 543 - DNA, 21 bp, synthetic construct
  source 1..21: mol_type = other DNA
  misc_feature 1..19: note = RNA
  misc_feature 20..21: note = DNA
ggctgagaaaatgatcttctt

SEQ ID NO: 544 - DNA, 21 bp, synthetic construct
  source 1..21: mol_type = other DNA
  misc_feature 1..19: note = RNA
  misc_feature 20..21: note = DNA
aagatcattttctcagccttt

SEQ ID NO: 545 - DNA, 21 bp, synthetic construct
  source 1..21: mol_type = other DNA
  misc_feature 1..19: note = RNA
  misc_feature 20..21: note = DNA
aggctgagaaaatgatctttt

SEQ ID NO: 546 - DNA, 21 bp, synthetic construct
  source 1..21: mol_type = other DNA
  misc_feature 1..19: note = RNA
  misc_feature 20..21: note = DNA
cattttctcagcctccagatt

SEQ ID NO: 547 - DNA, 21 bp, synthetic construct
  source 1..21: mol_type = other DNA
  misc_feature 1..19: note = RNA
  misc_feature 20..21: note = DNA
tctggaggctgagaaaatgtt

SEQ ID NO: 548 - DNA, 21 bp, synthetic construct
  source 1..21: mol_type = other DNA
  misc_feature 1..19: note = RNA
  misc_feature 20..21: note = DNA
agcctccagaggatgttcatt

SEQ ID NO: 549 - DNA, 21 bp, synthetic construct
  source 1..21: mol_type = other DNA
  misc_feature 1..19: note = RNA
  misc_feature 20..21: note = DNA
tgaacatcctctggaggcttt

SEQ ID NO: 550 - DNA, 21 bp, synthetic construct
  source 1..21: mol_type = other DNA
  misc_feature 1..19: note = RNA
  misc_feature 20..21: note = DNA
cctccagaggatgttcaattt

SEQ ID NO: 551 - DNA, 21 bp, synthetic construct
  source 1..21: mol_type = other DNA
  misc_feature 1..19: note = RNA
  misc_feature 20..21: note = DNA
attgaacatcctctggaggtt

SEQ ID NO: 552 - DNA, 21 bp, synthetic construct
  source 1..21: mol_type = other DNA
  misc_feature 1..19: note = RNA
  misc_feature 20..21: note = DNA
ctccagaggatgttcaatatt

SEQ ID NO: 553 - DNA, 21 bp, synthetic construct
  source 1..21: mol_type = other DNA
  misc_feature 1..19: note = RNA
  misc_feature 20..21: note = DNA
tattgaacatcctctggagtt

SEQ ID NO: 554 - DNA, 21 bp, synthetic construct
  source 1..21: mol_type = other DNA
  misc_feature 1..19: note = RNA
  misc_feature 20..21: note = DNA
agaggatgttcaataactgtt

SEQ ID NO: 555 - DNA, 21 bp, synthetic construct
  source 1..21: mol_type = other DNA
  misc_feature 1..19: note = RNA
  misc_feature 20..21: note = DNA
cagttattgaacatcctcttt

SEQ ID NO: 556 - DNA, 21 bp, synthetic construct
  source 1..21: mol_type = other DNA
  misc_feature 1..19: note = RNA
  misc_feature 20..21: note = DNA
aggatgttcaataactgtgtt

SEQ ID NO: 557 - DNA, 21 bp, synthetic construct
  source 1..21: mol_type = other DNA
  misc_feature 1..19: note = RNA
  misc_feature 20..21: note = DNA
cacagttattgaacatccttt

SEQ ID NO: 558 - DNA, 21 bp, synthetic construct
  source 1..21: mol_type = other DNA
  misc_feature 1..19: note = RNA
  misc_feature 20..21: note = DNA
tgttcaataactgtgaggttt

SEQ ID NO: 559 - DNA, 21 bp, synthetic construct
  source 1..21: mol_type = other DNA
  misc_feature 1..19: note = RNA
  misc_feature 20..21: note = DNA
acctcacagttattgaacatt

SEQ ID NO: 560 - DNA, 21 bp, synthetic construct
  source 1..21: mol_type = other DNA
  misc_feature 1..19: note = RNA
  misc_feature 20..21: note = DNA
ttcaataactgtgaggtggtt

SEQ ID NO: 561 - DNA, 21 bp, synthetic construct
  source 1..21: mol_type = other DNA
  misc_feature 1..19: note = RNA
  misc_feature 20..21: note = DNA
ccacctcacagttattgaatt

SEQ ID NO: 562 - DNA, 21 bp, synthetic construct
  source 1..21: mol_type = other DNA
  misc_feature 1..19: note = RNA
  misc_feature 20..21: note = DNA
tcaataactgtgaggtggttt

SEQ ID NO: 563 - DNA, 21 bp, synthetic construct
  source 1..21: mol_type = other DNA
  misc_feature 1..19: note = RNA
  misc_feature 20..21: note = DNA
accacctcacagttattgatt

SEQ ID NO: 564 - DNA, 21 bp, synthetic construct
  source 1..21: mol_type = other DNA
  misc_feature 1..19: note = RNA
  misc_feature 20..21: note = DNA
caataactgtgaggtggtctt

SEQ ID NO: 565 - DNA, 21 bp, synthetic construct
  source 1..21: mol_type = other DNA
  misc_feature 1..19: note = RNA
  misc_feature 20..21: note = DNA
gaccacctcacagttattgtt

SEQ ID NO: 566 - DNA, 21 bp, synthetic construct
  source 1..21: mol_type = other DNA
  misc_feature 1..19: note = RNA
  misc_feature 20..21: note = DNA
aataactgtgaggtggtcctt

SEQ ID NO: 567 - DNA, 21 bp, synthetic construct
  source 1..21: mol_type = other DNA
  misc_feature 1..19: note = RNA
  misc_feature 20..21: note = DNA
ggaccacctcacagttatttt

SEQ ID NO: 568 - DNA, 21 bp, synthetic construct
  source 1..21: mol_type = other DNA
  misc_feature 1..19: note = RNA
  misc_feature 20..21: note = DNA
ataactgtgaggtggtccttt

SEQ ID NO: 569 - DNA, 21 bp, synthetic construct
  source 1..21: mol_type = other DNA
  misc_feature 1..19: note = RNA
  misc_feature 20..21: note = DNA
aggaccacctcacagttattt

SEQ ID NO: 570 - DNA, 21 bp, synthetic construct
  source 1..21: mol_type = other DNA
  misc_feature 1..19: note = RNA
  misc_feature 20..21: note = DNA
taactgtgaggtggtcctttt

SEQ ID NO: 571 - DNA, 21 bp, synthetic construct
  source 1..21: mol_type = other DNA
  misc_feature 1..19: note = RNA
  misc_feature 20..21: note = DNA
aaggaccacctcacagttatt

SEQ ID NO: 572 - DNA, 21 bp, synthetic construct
  source 1..21: mol_type = other DNA
  misc_feature 1..19: note = RNA
  misc_feature 20..21: note = DNA
actgtgaggtggtccttggtt

SEQ ID NO: 573 - DNA, 21 bp, synthetic construct
  source 1..21: mol_type = other DNA
  misc_feature 1..19: note = RNA
  misc_feature 20..21: note = DNA
ccaaggaccacctcacagttt

SEQ ID NO: 574 - DNA, 21 bp, synthetic construct
  source 1..21: mol_type = other DNA
  misc_feature 1..19: note = RNA
  misc_feature 20..21: note = DNA
tgtgaggtggtccttgggatt

SEQ ID NO: 575 - DNA, 21 bp, synthetic construct
  source 1..21: mol_type = other DNA
  misc_feature 1..19: note = RNA
  misc_feature 20..21: note = DNA
tcccaaggaccacctcacatt

SEQ ID NO: 576 - DNA, 21 bp, synthetic construct
  source 1..21: mol_type = other DNA
  misc_feature 1..19: note = RNA
  misc_feature 20..21: note = DNA
gtgaggtggtccttgggaatt

SEQ ID NO: 577 - DNA, 21 bp, synthetic construct
  source 1..21: mol_type = other DNA
  misc_feature 1..19: note = RNA
  misc_feature 20..21: note = DNA
ttcccaaggaccacctcactt

SEQ ID NO: 578 - DNA, 21 bp, synthetic construct
  source 1..21: mol_type = other DNA
  misc_feature 1..19: note = RNA
  misc_feature 20..21: note = DNA
tggtccttgggaatttggatt

SEQ ID NO: 579 - DNA, 21 bp, synthetic construct
  source 1..21: mol_type = other DNA
  misc_feature 1..19: note = RNA
  misc_feature 20..21: note = DNA
tccaaattcccaaggaccatt

SEQ ID NO: 580 - DNA, 21 bp, synthetic construct
  source 1..21: mol_type = other DNA
  misc_feature 1..19: note = RNA
  misc_feature 20..21: note = DNA
ttgggaatttggaaattactt

SEQ ID NO: 581 - DNA, 21 bp, synthetic construct
  source 1..21: mol_type = other DNA
  misc_feature 1..19: note = RNA
  misc_feature 20..21: note = DNA
gtaatttccaaattcccaatt

SEQ ID NO: 582 - DNA, 21 bp, synthetic construct
  source 1..21: mol_type = other DNA
  misc_feature 1..19: note = RNA
  misc_feature 20..21: note = DNA
tgggaatttggaaattacctt

SEQ ID NO: 583 - DNA, 21 bp, synthetic construct
  source 1..21: mol_type = other DNA
  misc_feature 1..19: note = RNA
  misc_feature 20..21: note = DNA
ggtaatttccaaattcccatt

SEQ ID NO: 584 - DNA, 21 bp, synthetic construct
  source 1..21: mol_type = other DNA
  misc_feature 1..19: note = RNA
  misc_feature 20..21: note = DNA
gggaatttggaaattaccttt

SEQ ID NO: 585 - DNA, 21 bp, synthetic construct
  source 1..21: mol_type = other DNA
  misc_feature 1..19: note = RNA
  misc_feature 20..21: note = DNA
aggtaatttccaaattccctt

SEQ ID NO: 586 - DNA, 21 bp, synthetic construct
  source 1..21: mol_type = other DNA
  misc_feature 1..19: note = RNA
  misc_feature 20..21: note = DNA
ggaatttggaaattacctatt

SEQ ID NO: 587 - DNA, 21 bp, synthetic construct
  source 1..21: mol_type = other DNA
  misc_feature 1..19: note = RNA
  misc_feature 20..21: note = DNA
taggtaatttccaaattcctt

SEQ ID NO: 588 - DNA, 21 bp, synthetic construct
  source 1..21: mol_type = other DNA
  misc_feature 1..19: note = RNA
  misc_feature 20..21: note = DNA
agaggaattatgatctttctt

SEQ ID NO: 589 - DNA, 21 bp, synthetic construct
  source 1..21: mol_type = other DNA
  misc_feature 1..19: note = RNA
  misc_feature 20..21: note = DNA
gaaagatcataattcctcttt

SEQ ID NO: 590 - DNA, 21 bp, synthetic construct
  source 1..21: mol_type = other DNA
  misc_feature 1..19: note = RNA
  misc_feature 20..21: note = DNA
gaggaattatgatctttcctt

SEQ ID NO: 591 - DNA, 21 bp, synthetic construct
  source 1..21: mol_type = other DNA
  misc_feature 1..19: note = RNA
  misc_feature 20..21: note = DNA
ggaaagatcataattcctctt

SEQ ID NO: 592 - DNA, 21 bp, synthetic construct
  source 1..21: mol_type = other DNA
  misc_feature 1..19: note = RNA
  misc_feature 20..21: note = DNA
aattatgatctttccttcttt

SEQ ID NO: 593 - DNA, 21 bp, synthetic construct
  source 1..21: mol_type = other DNA
  misc_feature 1..19: note = RNA
  misc_feature 20..21: note = DNA
agaaggaaagatcataatttt

SEQ ID NO: 594 - DNA, 21 bp, synthetic construct
  source 1..21: mol_type = other DNA
  misc_feature 1..19: note = RNA
  misc_feature 20..21: note = DNA
attatgatctttccttctttt

SEQ ID NO: 595 - DNA, 21 bp, synthetic construct
  source 1..21: mol_type = other DNA
  misc_feature 1..19: note = RNA
  misc_feature 20..21: note = DNA
aagaaggaaagatcataattt

SEQ ID NO: 596 - DNA, 21 bp, synthetic construct
  source 1..21: mol_type = other DNA
  misc_feature 1..19: note = RNA
  misc_feature 20..21: note = DNA
tatgatctttccttcttaatt

SEQ ID NO: 597 - DNA, 21 bp, synthetic construct
  source 1..21: mol_type = other DNA
  misc_feature 1..19: note = RNA
  misc_feature 20..21: note = DNA
ttaagaaggaaagatcatatt

SEQ ID NO: 598 - DNA, 21 bp, synthetic construct
  source 1..21: mol_type = other DNA
  misc_feature 1..19: note = RNA
  misc_feature 20..21: note = DNA
tgatctttccttcttaaagtt

SEQ ID NO: 599 - DNA, 21 bp, synthetic construct
  source 1..21: mol_type = other DNA
  misc_feature 1..19: note = RNA
  misc_feature 20..21: note = DNA
ctttaagaaggaaagatcatt

SEQ ID NO: 600 - DNA, 21 bp, synthetic construct
  source 1..21: mol_type = other DNA
  misc_feature 1..19: note = RNA
  misc_feature 20..21: note = DNA
tctttccttcttaaagacctt

SEQ ID NO: 601 - DNA, 21 bp, synthetic construct
  source 1..21: mol_type = other DNA
  misc_feature 1..19: note = RNA
  misc_feature 20..21: note = DNA
ggtctttaagaaggaaagatt

SEQ ID NO: 602 - DNA, 21 bp, synthetic construct
  source 1..21: mol_type = other DNA
  misc_feature 1..19: note = RNA
  misc_feature 20..21: note = DNA
ccttcttaaagaccatccatt

SEQ ID NO: 603 - DNA, 21 bp, synthetic construct
  source 1..21: mol_type = other DNA
  misc_feature 1..19: note = RNA
  misc_feature 20..21: note = DNA
tggatggtctttaagaaggtt

SEQ ID NO: 604 - DNA, 21 bp, synthetic construct
  source 1..21: mol_type = other DNA
  misc_feature 1..19: note = RNA
  misc_feature 20..21: note = DNA
cttcttaaagaccatccagtt

SEQ ID NO: 605 - DNA, 21 bp, synthetic construct
  source 1..21: mol_type = other DNA
  misc_feature 1..19: note = RNA
  misc_feature 20..21: note = DNA
ctggatggtctttaagaagtt

SEQ ID NO: 606 - DNA, 21 bp, synthetic construct
  source 1..21: mol_type = other DNA
  misc_feature 1..19: note = RNA
  misc_feature 20..21: note = DNA
tcttaaagaccatccaggatt

SEQ ID NO: 607 - DNA, 21 bp, synthetic construct
  source 1..21: mol_type = other DNA
  misc_feature 1..19: note = RNA
  misc_feature 20..21: note = DNA
tcctggatggtctttaagatt

SEQ ID NO: 608 - DNA, 21 bp, synthetic construct
  source 1..21: mol_type = other DNA
  misc_feature 1..19: note = RNA
  misc_feature 20..21: note = DNA
ttaaagaccatccaggaggtt

SEQ ID NO: 609 - DNA, 21 bp, synthetic construct
  source 1..21: mol_type = other DNA
  misc_feature 1..19: note = RNA
  misc_feature 20..21: note = DNA
cctcctggatggtctttaatt

SEQ ID NO: 610 - DNA, 21 bp, synthetic construct
  source 1..21: mol_type = other DNA
  misc_feature 1..19: note = RNA
  misc_feature 20..21: note = DNA
taaagaccatccaggaggttt

SEQ ID NO: 611 - DNA, 21 bp, synthetic construct
  source 1..21: mol_type = other DNA
  misc_feature 1..19: note = RNA
  misc_feature 20..21: note = DNA
acctcctggatggtctttatt

SEQ ID NO: 612 - DNA, 21 bp, synthetic construct
  source 1..21: mol_type = other DNA
  misc_feature 1..19: note = RNA
  misc_feature 20..21: note = DNA
ccaggaggtggctggttattt

SEQ ID NO: 613 - DNA, 21 bp, synthetic construct
  source 1..21: mol_type = other DNA
  misc_feature 1..19: note = RNA
  misc_feature 20..21: note = DNA
ataaccagccacctcctggtt

SEQ ID NO: 614 - DNA, 21 bp, synthetic construct
  source 1..21: mol_type = other DNA
  misc_feature 1..19: note = RNA
  misc_feature 20..21: note = DNA
caggaggtggctggttatgtt

SEQ ID NO: 615 - DNA, 21 bp, synthetic construct
  source 1..21: mol_type = other DNA
  misc_feature 1..19: note = RNA
  misc_feature 20..21: note = DNA
cataaccagccacctcctgtt

SEQ ID NO: 616 - DNA, 21 bp, synthetic construct
  source 1..21: mol_type = other DNA
  misc_feature 1..19: note = RNA
  misc_feature 20..21: note = DNA
aggaggtggctggttatgttt

SEQ ID NO: 617 - DNA, 21 bp, synthetic construct
  source 1..21: mol_type = other DNA
  misc_feature 1..19: note = RNA
  misc_feature 20..21: note = DNA
acataaccagccacctccttt

SEQ ID NO: 618 - DNA, 21 bp, synthetic construct
  source 1..21: mol_type = other DNA
  misc_feature 1..19: note = RNA
  misc_feature 20..21: note = DNA
ggaggtggctggttatgtctt

SEQ ID NO: 619 - DNA, 21 bp, synthetic construct
  source 1..21: mol_type = other DNA
  misc_feature 1..19: note = RNA
  misc_feature 20..21: note = DNA
gacataaccagccacctcctt

SEQ ID NO: 620 - DNA, 21 bp, synthetic construct
  source 1..21: mol_type = other DNA
  misc_feature 1..19: note = RNA
  misc_feature 20..21: note = DNA
gaggtggctggttatgtcctt

SEQ ID NO: 621 - DNA, 21 bp, synthetic construct
  source 1..21: mol_type = other DNA
  misc_feature 1..19: note = RNA
  misc_feature 20..21: note = DNA
ggacataaccagccacctctt

SEQ ID NO: 622 - DNA, 21 bp, synthetic construct
  source 1..21: mol_type = other DNA
  misc_feature 1..19: note = RNA
  misc_feature 20..21: note = DNA
aggtggctggttatgtccttt

SEQ ID NO: 623 - DNA, 21 bp, synthetic construct
  source 1..21: mol_type = other DNA
  misc_feature 1..19: note = RNA
  misc_feature 20..21: note = DNA
aggacataaccagccaccttt

SEQ ID NO: 624 - DNA, 21 bp, synthetic construct
  source 1..21: mol_type = other DNA
  misc_feature 1..19: note = RNA
  misc_feature 20..21: note = DNA
gtggctggttatgtcctcatt

SEQ ID NO: 625 - DNA, 21 bp, synthetic construct
  source 1..21: mol_type = other DNA
  misc_feature 1..19: note = RNA
  misc_feature 20..21: note = DNA
tgaggacataaccagccactt

SEQ ID NO: 626 - DNA, 21 bp, synthetic construct
  source 1..21: mol_type = other DNA
  misc_feature 1..19: note = RNA
  misc_feature 20..21: note = DNA
gccctcaacacagtggagctt

SEQ ID NO: 627 - DNA, 21 bp, synthetic construct
  source 1..21: mol_type = other DNA
  misc_feature 1..19: note = RNA
  misc_feature 20..21: note = DNA
gctccactgtgttgagggctt

SEQ ID NO: 628 - DNA, 21 bp, synthetic construct
  source 1..21: mol_type = other DNA
  misc_feature 1..19: note = RNA
  misc_feature 20..21: note = DNA
ttcctttggaaaacctgcatt

SEQ ID NO: 629 - DNA, 21 bp, synthetic construct
  source 1..21: mol_type = other DNA
  misc_feature 1..19: note = RNA
  misc_feature 20..21: note = DNA
tgcaggttttccaaaggaatt

SEQ ID NO: 630 - DNA, 21 bp, synthetic construct
  source 1..21: mol_type = other DNA
  misc_feature 1..19: note = RNA
  misc_feature 20..21: note = DNA
tcctttggaaaacctgcagtt

SEQ ID NO: 631 - DNA, 21 bp, synthetic construct
  source 1..21: mol_type = other DNA
  misc_feature 1..19: note = RNA
  misc_feature 20..21: note = DNA
ctgcaggttttccaaaggatt

SEQ ID NO: 632 - DNA, 21 bp, synthetic construct
  source 1..21: mol_type = other DNA
  misc_feature 1..19: note = RNA
  misc_feature 20..21: note = DNA
gaaaacctgcagatcatcatt

SEQ ID NO: 633 - DNA, 21 bp, synthetic construct
  source 1..21: mol_type = other DNA
  misc_feature 1..19: note = RNA
  misc_feature 20..21: note = DNA
tgatgatctgcaggttttctt

SEQ ID NO: 634 - DNA, 21 bp, synthetic construct
  source 1..21: mol_type = other DNA
  misc_feature 1..19: note = RNA
  misc_feature 20..21: note = DNA
aaaacctgcagatcatcagtt

SEQ ID NO: 635 - DNA, 21 bp, synthetic construct
  source 1..21: mol_type = other DNA
  misc_feature 1..19: note = RNA
  misc_feature 20..21: note = DNA
ctgatgatctgcaggtttttt

SEQ ID NO: 636 - DNA, 21 bp, synthetic construct
  source 1..21: mol_type = other DNA
  misc_feature 1..19: note = RNA
  misc_feature 20..21: note = DNA
aacctgcagatcatcagagtt

SEQ ID NO: 637 - DNA, 21 bp, synthetic construct
  source 1..21: mol_type = other DNA
  misc_feature 1..19: note = RNA
  misc_feature 20..21: note = DNA
ctctgatgatctgcaggtttt

SEQ ID NO: 638 - DNA, 21 bp, synthetic construct
  source 1..21: mol_type = other DNA
  misc_feature 1..19: note = RNA
  misc_feature 20..21: note = DNA
ctgcagatcatcagaggaatt

SEQ ID NO: 639 - DNA, 21 bp, synthetic construct
  source 1..21: mol_type = other DNA
  misc_feature 1..19: note = RNA
  misc_feature 20..21: note = DNA
ttcctctgatgatctgcagtt

SEQ ID NO: 640 - DNA, 21 bp, synthetic construct
  source 1..21: mol_type = other DNA
  misc_feature 1..19: note = RNA
  misc_feature 20..21: note = DNA
gaaaattcctatgccttagtt

SEQ ID NO: 641 - DNA, 21 bp, synthetic construct
  source 1..21: mol_type = other DNA
  misc_feature 1..19: note = RNA
  misc_feature 20..21: note = DNA
ctaaggcataggaattttctt

SEQ ID NO: 642 - DNA, 21 bp, synthetic construct
  source 1..21: mol_type = other DNA
  misc_feature 1..19: note = RNA
  misc_feature 20..21: note = DNA
aaaattcctatgccttagctt

SEQ ID NO: 643 - DNA, 21 bp, synthetic construct
  source 1..21: mol_type = other DNA
  misc_feature 1..19: note = RNA
  misc_feature 20..21: note = DNA
gctaaggcataggaatttttt

SEQ ID NO: 644 - DNA, 21 bp, synthetic construct
  source 1..21: mol_type = other DNA
  misc_feature 1..19: note = RNA
  misc_feature 20..21: note = DNA
aattcctatgccttagcagtt

SEQ ID NO: 645 - DNA, 21 bp, synthetic construct
  source 1..21: mol_type = other DNA
  misc_feature 1..19: note = RNA
  misc_feature 20..21: note = DNA
ctgctaaggcataggaatttt

SEQ ID NO: 646 - DNA, 21 bp, synthetic construct
  source 1..21: mol_type = other DNA
  misc_feature 1..19: note = RNA
  misc_feature 20..21: note = DNA
tcctatgccttagcagtcttt

SEQ ID NO: 647 - DNA, 21 bp, synthetic construct
  source 1..21: mol_type = other DNA
  misc_feature 1..19: note = RNA
  misc_feature 20..21: note = DNA
agactgctaaggcataggatt

SEQ ID NO: 648 - DNA, 21 bp, synthetic construct
  source 1..21: mol_type = other DNA
  misc_feature 1..19: note = RNA
  misc_feature 20..21: note = DNA
cctatgccttagcagtctttt

SEQ ID NO: 649 - DNA, 21 bp, synthetic construct
  source 1..21: mol_type = other DNA
  misc_feature 1..19: note = RNA
  misc_feature 20..21: note = DNA
aagactgctaaggcataggtt

SEQ ID NO: 650 - DNA, 21 bp, synthetic construct
  source 1..21: mol_type = other DNA
  misc_feature 1..19: note = RNA
  misc_feature 20..21: note = DNA
ctatgccttagcagtcttatt

SEQ ID NO: 651 - DNA, 21 bp, synthetic construct
  source 1..21: mol_type = other DNA
  misc_feature 1..19: note = RNA
  misc_feature 20..21: note = DNA
taagactgctaaggcatagtt

SEQ ID NO: 652 - DNA, 21 bp, synthetic construct
  source 1..21: mol_type = other DNA
  misc_feature 1..19: note = RNA
  misc_feature 20..21: note = DNA
atgccttagcagtcttatctt

SEQ ID NO: 653 - DNA, 21 bp, synthetic construct
  source 1..21: mol_type = other DNA
  misc_feature 1..19: note = RNA
  misc_feature 20..21: note = DNA
gataagactgctaaggcattt

SEQ ID NO: 654 - DNA, 21 bp, synthetic construct
  source 1..21: mol_type = other DNA
  misc_feature 1..19: note = RNA
  misc_feature 20..21: note = DNA
tgccttagcagtcttatcttt

SEQ ID NO: 655 - DNA, 21 bp, synthetic construct
  source 1..21: mol_type = other DNA
  misc_feature 1..19: note = RNA
  misc_feature 20..21: note = DNA
agataagactgctaaggcatt

SEQ ID NO: 656 - DNA, 21 bp, synthetic construct
  source 1..21: mol_type = other DNA
  misc_feature 1..19: note = RNA
  misc_feature 20..21: note = DNA
gccttagcagtcttatctatt

SEQ ID NO: 657 - DNA, 21 bp, synthetic construct
  source 1..21: mol_type = other DNA
  misc_feature 1..19: note = RNA
  misc_feature 20..21: note = DNA
tagataagactgctaaggctt

SEQ ID NO: 658 - DNA, 21 bp, synthetic construct
  source 1..21: mol_type = other DNA
  misc_feature 1..19: note = RNA
  misc_feature 20..21: note = DNA
ccttagcagtcttatctaatt

SEQ ID NO: 659 - DNA, 21 bp, synthetic construct
  source 1..21: mol_type = other DNA
  misc_feature 1..19: note = RNA
  misc_feature 20..21: note = DNA
ttagataagactgctaaggtt

SEQ ID NO: 660 - DNA, 21 bp, synthetic construct
  source 1..21: mol_type = other DNA
  misc_feature 1..19: note = RNA
  misc_feature 20..21: note = DNA
cttagcagtcttatctaactt

SEQ ID NO: 661 - DNA, 21 bp, synthetic construct
  source 1..21: mol_type = other DNA
  misc_feature 1..19: note = RNA
  misc_feature 20..21: note = DNA
gttagataagactgctaagtt

SEQ ID NO: 662 - DNA, 21 bp, synthetic construct
  source 1..21: mol_type = other DNA
  misc_feature 1..19: note = RNA
  misc_feature 20..21: note = DNA
ttagcagtcttatctaacttt

SEQ ID NO: 663 - DNA, 21 bp, synthetic construct
  source 1..21: mol_type = other DNA
  misc_feature 1..19: note = RNA
  misc_feature 20..21: note = DNA
agttagataagactgctaatt

SEQ ID NO: 664 - DNA, 21 bp, synthetic construct
  source 1..21: mol_type = other DNA
  misc_feature 1..19: note = RNA
  misc_feature 20..21: note = DNA
tagcagtcttatctaactatt

SEQ ID NO: 665 - DNA, 21 bp, synthetic construct
  source 1..21: mol_type = other DNA
  misc_feature 1..19: note = RNA
  misc_feature 20..21: note = DNA
tagttagataagactgctatt

SEQ ID NO: 666 - DNA, 21 bp, synthetic construct
  source 1..21: mol_type = other DNA
  misc_feature 1..19: note = RNA
  misc_feature 20..21: note = DNA
agcagtcttatctaactattt

SEQ ID NO: 667 - DNA, 21 bp, synthetic construct
  source 1..21: mol_type = other DNA
  misc_feature 1..19: note = RNA
  misc_feature 20..21: note = DNA
atagttagataagactgcttt

SEQ ID NO: 668 - DNA, 21 bp, synthetic construct
  source 1..21: mol_type = other DNA
  misc_feature 1..19: note = RNA
  misc_feature 20..21: note = DNA
gcagtcttatctaactatgtt

SEQ ID NO: 669 - DNA, 21 bp, synthetic construct
  source 1..21: mol_type = other DNA
  misc_feature 1..19: note = RNA
  misc_feature 20..21: note = DNA
catagttagataagactgctt

SEQ ID NO: 670 - DNA, 21 bp, synthetic construct
  source 1..21: mol_type = other DNA
  misc_feature 1..19: note = RNA
  misc_feature 20..21: note = DNA
cagtcttatctaactatgatt

SEQ ID NO: 671 - DNA, 21 bp, synthetic construct
  source 1..21: mol_type = other DNA
  misc_feature 1..19: note = RNA
  misc_feature 20..21: note = DNA
tcatagttagataagactgtt

SEQ ID NO: 672 - DNA, 21 bp, synthetic construct
  source 1..21: mol_type = other DNA
  misc_feature 1..19: note = RNA
  misc_feature 20..21: note = DNA
tcttatctaactatgatgctt

SEQ ID NO: 673 - DNA, 21 bp, synthetic construct
  source 1..21: mol_type = other DNA
  misc_feature 1..19: note = RNA
  misc_feature 20..21: note = DNA
gcatcatagttagataagatt

SEQ ID NO: 674 - DNA, 21 bp, synthetic construct
  source 1..21: mol_type = other DNA
  misc_feature 1..19: note = RNA
  misc_feature 20..21: note = DNA
cttatctaactatgatgcatt

SEQ ID NO: 675 - DNA, 21 bp, synthetic construct
  source 1..21: mol_type = other DNA
  misc_feature 1..19: note = RNA
  misc_feature 20..21: note = DNA
tgcatcatagttagataagtt

SEQ ID NO: 676 - DNA, 21 bp, synthetic construct
  source 1..21: mol_type = other DNA
  misc_feature 1..19: note = RNA
  misc_feature 20..21: note = DNA
ttatctaactatgatgcaatt

SEQ ID NO: 677 - DNA, 21 bp, synthetic construct
  source 1..21: mol_type = other DNA
  misc_feature 1..19: note = RNA
  misc_feature 20..21: note = DNA
ttgcatcatagttagataatt

SEQ ID NO: 678 - DNA, 21 bp, synthetic construct
  source 1..21: mol_type = other DNA
  misc_feature 1..19: note = RNA
  misc_feature 20..21: note = DNA
tatctaactatgatgcaaatt

SEQ ID NO: 679 - DNA, 21 bp, synthetic construct
  source 1..21: mol_type = other DNA
  misc_feature 1..19: note = RNA
  misc_feature 20..21: note = DNA
tttgcatcatagttagatatt

SEQ ID NO: 680 - DNA, 21 bp, synthetic construct
  source 1..21: mol_type = other DNA
  misc_feature 1..19: note = RNA
  misc_feature 20..21: note = DNA
atctaactatgatgcaaattt

SEQ ID NO: 681 - DNA, 21 bp, synthetic construct
  source 1..21: mol_type = other DNA
  misc_feature 1..19: note = RNA
  misc_feature 20..21: note = DNA
atttgcatcatagttagattt

SEQ ID NO: 682 - DNA, 21 bp, synthetic construct
  source 1..21: mol_type = other DNA
  misc_feature 1..19: note = RNA
  misc_feature 20..21: note = DNA
tctaactatgatgcaaatatt

SEQ ID NO: 683 - DNA, 21 bp, synthetic construct
  source 1..21: mol_type = other DNA
  misc_feature 1..19: note = RNA
  misc_feature 20..21: note = DNA
tatttgcatcatagttagatt

SEQ ID NO: 684 - DNA, 21 bp, synthetic construct
  source 1..21: mol_type = other DNA
  misc_feature 1..19: note = RNA
  misc_feature 20..21: note = DNA
ctaactatgatgcaaataatt

SEQ ID NO: 685 - DNA, 21 bp, synthetic construct
  source 1..21: mol_type = other DNA
  misc_feature 1..19: note = RNA
  misc_feature 20..21: note = DNA
ttatttgcatcatagttagtt

SEQ ID NO: 686 - DNA, 21 bp, synthetic construct
  source 1..21: mol_type = other DNA
  misc_feature 1..19: note = RNA
  misc_feature 20..21: note = DNA
aactatgatgcaaataaaatt

SEQ ID NO: 687 - DNA, 21 bp, synthetic construct
  source 1..21: mol_type = other DNA
  misc_feature 1..19: note = RNA
  misc_feature 20..21: note = DNA
ttttatttgcatcatagtttt

SEQ ID NO: 688 - DNA, 21 bp, synthetic construct
  source 1..21: mol_type = other DNA
  misc_feature 1..19: note = RNA
  misc_feature 20..21: note = DNA
gatgcaaataaaaccggactt

SEQ ID NO: 689 - DNA, 21 bp, synthetic construct
  source 1..21: mol_type = other DNA
  misc_feature 1..19: note = RNA
  misc_feature 20..21: note = DNA
gtccggttttatttgcatctt

SEQ ID NO: 690 - DNA, 21 bp, synthetic construct
  source 1..21: mol_type = other DNA
  misc_feature 1..19: note = RNA
  misc_feature 20..21: note = DNA
atgcaaataaaaccggacttt

SEQ ID NO: 691 - DNA, 21 bp, synthetic construct
  source 1..21: mol_type = other DNA
  misc_feature 1..19: note = RNA
  misc_feature 20..21: note = DNA
agtccggttttatttgcattt

SEQ ID NO: 692 - DNA, 21 bp, synthetic construct
  source 1..21: mol_type = other DNA
  misc_feature 1..19: note = RNA
  misc_feature 20..21: note = DNA
tgcaaataaaaccggactgtt

SEQ ID NO: 693 - DNA, 21 bp, synthetic construct
  source 1..21: mol_type = other DNA
  misc_feature 1..19: note = RNA
  misc_feature 20..21: note = DNA
cagtccggttttatttgcatt

SEQ ID NO: 694 - DNA, 21 bp, synthetic construct
  source 1..21: mol_type = other DNA
  misc_feature 1..19: note = RNA
  misc_feature 20..21: note = DNA
caaataaaaccggactgaatt

SEQ ID NO: 695 - DNA, 21 bp, synthetic construct
  source 1..21: mol_type = other DNA
  misc_feature 1..19: note = RNA
  misc_feature 20..21: note = DNA
ttcagtccggttttatttgtt

SEQ ID NO: 696 - DNA, 21 bp, synthetic construct
  source 1..21: mol_type = other DNA
  misc_feature 1..19: note = RNA
  misc_feature 20..21: note = DNA
aaataaaaccggactgaagtt

SEQ ID NO: 697 - DNA, 21 bp, synthetic construct
  source 1..21: mol_type = other DNA
  misc_feature 1..19: note = RNA
  misc_feature 20..21: note = DNA
cttcagtccggttttattttt

SEQ ID NO: 698 - DNA, 21 bp, synthetic construct
  source 1..21: mol_type = other DNA
  misc_feature 1..19: note = RNA
  misc_feature 20..21: note = DNA
aataaaaccggactgaaggtt

SEQ ID NO: 699 - DNA, 21 bp, synthetic construct
  source 1..21: mol_type = other DNA
  misc_feature 1..19: note = RNA
  misc_feature 20..21: note = DNA
ccttcagtccggttttatttt

SEQ ID NO: 700 - DNA, 21 bp, synthetic construct
  source 1..21: mol_type = other DNA
  misc_feature 1..19: note = RNA
  misc_feature 20..21: note = DNA
taaaaccggactgaaggagtt

SEQ ID NO: 701 - DNA, 21 bp, synthetic construct
  source 1..21: mol_type = other DNA
  misc_feature 1..19: note = RNA
  misc_feature 20..21: note = DNA
ctccttcagtccggttttatt

SEQ ID NO: 702 - DNA, 21 bp, synthetic construct
  source 1..21: mol_type = other DNA
  misc_feature 1..19: note = RNA
  misc_feature 20..21: note = DNA
aaaaccggactgaaggagctt

SEQ ID NO: 703 - DNA, 21 bp, synthetic construct
  source 1..21: mol_type = other DNA
  misc_feature 1..19: note = RNA
  misc_feature 20..21: note = DNA
gctccttcagtccggtttttt

SEQ ID NO: 704 - DNA, 21 bp, synthetic construct
  source 1..21: mol_type = other DNA
  misc_feature 1..19: note = RNA
  misc_feature 20..21: note = DNA
aaaccggactgaaggagcttt

SEQ ID NO: 705 - DNA, 21 bp, synthetic construct
  source 1..21: mol_type = other DNA
  misc_feature 1..19: note = RNA
  misc_feature 20..21: note = DNA
agctccttcagtccggttttt

SEQ ID NO: 706 - DNA, 21 bp, synthetic construct
  source 1..21: mol_type = other DNA
  misc_feature 1..19: note = RNA
  misc_feature 20..21: note = DNA
aaccggactgaaggagctgtt

SEQ ID NO: 707 - DNA, 21 bp, synthetic construct
  source 1..21: mol_type = other DNA
  misc_feature 1..19: note = RNA
  misc_feature 20..21: note = DNA
cagctccttcagtccggtttt

SEQ ID NO: 708 - DNA, 21 bp, synthetic construct
  source 1..21: mol_type = other DNA
  misc_feature 1..19: note = RNA
  misc_feature 20..21: note = DNA
aggagctgcccatgagaaatt

SEQ ID NO: 709 - DNA, 21 bp, synthetic construct
  source 1..21: mol_type = other DNA
  misc_feature 1..19: note = RNA
  misc_feature 20..21: note = DNA
tttctcatgggcagctccttt

SEQ ID NO: 710 - DNA, 21 bp, synthetic construct
  source 1..21: mol_type = other DNA
  misc_feature 1..19: note = RNA
  misc_feature 20..21: note = DNA
tacaggaaatcctgcatggtt

SEQ ID NO: 711 - DNA, 21 bp, synthetic construct
  source 1..21: mol_type = other DNA
  misc_feature 1..19: note = RNA
  misc_feature 20..21: note = DNA
ccatgcaggatttcctgtatt

SEQ ID NO: 712 - DNA, 21 bp, synthetic construct
  source 1..21: mol_type = other DNA
  misc_feature 1..19: note = RNA
  misc_feature 20..21: note = DNA
aggaaatcctgcatggcgctt

SEQ ID NO: 713 - DNA, 21 bp, synthetic construct
  source 1..21: mol_type = other DNA
  misc_feature 1..19: note = RNA
  misc_feature 20..21: note = DNA
gcgccatgcaggatttccttt

SEQ ID NO: 714 - DNA, 21 bp, synthetic construct
  source 1..21: mol_type = other DNA
  misc_feature 1..19: note = RNA
  misc_feature 20..21: note = DNA
ggaaatcctgcatggcgcctt

SEQ ID NO: 715 - DNA, 21 bp, synthetic construct
  source 1..21: mol_type = other DNA
  misc_feature 1..19: note = RNA
  misc_feature 20..21: note = DNA
ggcgccatgcaggatttcctt

SEQ ID NO: 716 - DNA, 21 bp, synthetic construct
  source 1..21: mol_type = other DNA
  misc_feature 1..19: note = RNA
  misc_feature 20..21: note = DNA
gaaatcctgcatggcgccgtt

SEQ ID NO: 717 - DNA, 21 bp, synthetic construct
  source 1..21: mol_type = other DNA
  misc_feature 1..19: note = RNA
  misc_feature 20..21: note = DNA
cggcgccatgcaggatttctt

SEQ ID NO: 718 - DNA, 21 bp, synthetic construct
  source 1..21: mol_type = other DNA
  misc_feature 1..19: note = RNA
  misc_feature 20..21: note = DNA
aaatcctgcatggcgccgttt

SEQ ID NO: 719 - DNA, 21 bp, synthetic construct
  source 1..21: mol_type = other DNA
  misc_feature 1..19: note = RNA
  misc_feature 20..21: note = DNA
acggcgccatgcaggattttt

SEQ ID NO: 720 - DNA, 21 bp, synthetic construct
  source 1..21: mol_type = other DNA
  misc_feature 1..19: note = RNA
  misc_feature 20..21: note = DNA
aatcctgcatggcgccgtgtt

SEQ ID NO: 721 - DNA, 21 bp, synthetic construct
  source 1..21: mol_type = other DNA
  misc_feature 1..19: note = RNA
  misc_feature 20..21: note = DNA
cacggcgccatgcaggatttt

SEQ ID NO: 722 - DNA, 21 bp, synthetic construct
  source 1..21: mol_type = other DNA
  misc_feature 1..19: note = RNA
  misc_feature 20..21: note = DNA
tcctgcatggcgccgtgcgtt

SEQ ID NO: 723 - DNA, 21 bp, synthetic construct
  source 1..21: mol_type = other DNA
  misc_feature 1..19: note = RNA
  misc_feature 20..21: note = DNA
cgcacggcgccatgcaggatt

SEQ ID NO: 724 - DNA, 21 bp, synthetic construct
  source 1..21: mol_type = other DNA
  misc_feature 1..19: note = RNA
  misc_feature 20..21: note = DNA
ctgcatggcgccgtgcggttt

SEQ ID NO: 725 - DNA, 21 bp, synthetic construct
  source 1..21: mol_type = other DNA
  misc_feature 1..19: note = RNA
  misc_feature 20..21: note = DNA
accgcacggcgccatgcagtt

SEQ ID NO: 726 - DNA, 21 bp, synthetic construct
  source 1..21: mol_type = other DNA
  misc_feature 1..19: note = RNA
  misc_feature 20..21: note = DNA
tgcatggcgccgtgcggtttt

SEQ ID NO: 727 - DNA, 21 bp, synthetic construct
  source 1..21: mol_type = other DNA
  misc_feature 1..19: note = RNA
  misc_feature 20..21: note = DNA
aaccgcacggcgccatgcatt

SEQ ID NO: 728 - DNA, 21 bp, synthetic construct
  source 1..21: mol_type = other DNA
  misc_feature 1..19: note = RNA
  misc_feature 20..21: note = DNA
gcatggcgccgtgcggttctt

SEQ ID NO: 729 - DNA, 21 bp, synthetic construct
  source 1..21: mol_type = other DNA
  misc_feature 1..19: note = RNA
  misc_feature 20..21: note = DNA
gaaccgcacggcgccatgctt

SEQ ID NO: 730 - DNA, 21 bp, synthetic construct
  source 1..21: mol_type = other DNA
  misc_feature 1..19: note = RNA
  misc_feature 20..21: note = DNA
atggcgccgtgcggttcagtt

SEQ ID NO: 731 - DNA, 21 bp, synthetic construct
  source 1..21: mol_type = other DNA
  misc_feature 1..19: note = RNA
  misc_feature 20..21: note = DNA
ctgaaccgcacggcgccattt

SEQ ID NO: 732 - DNA, 21 bp, synthetic construct
  source 1..21: mol_type = other DNA
  misc_feature 1..19: note = RNA
  misc_feature 20..21: note = DNA
tggcgccgtgcggttcagctt

SEQ ID NO: 733 - DNA, 21 bp, synthetic construct
  source 1..21: mol_type = other DNA
  misc_feature 1..19: note = RNA
  misc_feature 20..21: note = DNA
gctgaaccgcacggcgccatt

SEQ ID NO: 734 - DNA, 21 bp, synthetic construct
  source 1..21: mol_type = other DNA
  misc_feature 1..19: note = RNA
  misc_feature 20..21: note = DNA
ggcgccgtgcggttcagcatt

SEQ ID NO: 735 - DNA, 21 bp, synthetic construct
  source 1..21: mol_type = other DNA
  misc_feature 1..19: note = RNA
  misc_feature 20..21: note = DNA
tgctgaaccgcacggcgcctt

SEQ ID NO: 736 - DNA, 21 bp, synthetic construct
  source 1..21: mol_type = other DNA
  misc_feature 1..19: note = RNA
  misc_feature 20..21: note = DNA
gcgccgtgcggttcagcaatt

SEQ ID NO: 737 - DNA, 21 bp, synthetic construct
  source 1..21: mol_type = other DNA
  misc_feature 1..19: note = RNA
  misc_feature 20..21: note = DNA
ttgctgaaccgcacggcgctt

SEQ ID NO: 738 - DNA, 21 bp, synthetic construct
  source 1..21: mol_type = other DNA
  misc_feature 1..19: note = RNA
  misc_feature 20..21: note = DNA
cgccgtgcggttcagcaactt

SEQ ID NO: 739 - DNA, 21 bp, synthetic construct
  source 1..21: mol_type = other DNA
  misc_feature 1..19: note = RNA
  misc_feature 20..21: note = DNA
gttgctgaaccgcacggcgtt

SEQ ID NO: 740 - DNA, 21 bp, synthetic construct
  source 1..21: mol_type = other DNA
  misc_feature 1..19: note = RNA
  misc_feature 20..21: note = DNA
gccgtgcggttcagcaacatt

SEQ ID NO: 741 - DNA, 21 bp, synthetic construct
  source 1..21: mol_type = other DNA
  misc_feature 1..19: note = RNA
  misc_feature 20..21: note = DNA
tgttgctgaaccgcacggctt

SEQ ID NO: 742 - DNA, 21 bp, synthetic construct
  source 1..21: mol_type = other DNA
  misc_feature 1..19: note = RNA
  misc_feature 20..21: note = DNA
ccgtgcggttcagcaacaatt

SEQ ID NO: 743 - DNA, 21 bp, synthetic construct
  source 1..21: mol_type = other DNA
  misc_feature 1..19: note = RNA
  misc_feature 20..21: note = DNA
ttgttgctgaaccgcacggtt

SEQ ID NO: 744 - DNA, 21 bp, synthetic construct
  source 1..21: mol_type = other DNA
  misc_feature 1..19: note = RNA
  misc_feature 20..21: note = DNA
gtgcggttcagcaacaacctt

SEQ ID NO: 745 - DNA, 21 bp, synthetic construct
  source 1..21: mol_type = other DNA
  misc_feature 1..19: note = RNA
  misc_feature 20..21: note = DNA
ggttgttgctgaaccgcactt

SEQ ID NO: 746 - DNA, 21 bp, synthetic construct
  source 1..21: mol_type = other DNA
  misc_feature 1..19: note = RNA
  misc_feature 20..21: note = DNA
gcggttcagcaacaacccttt

SEQ ID NO: 747 - DNA, 21 bp, synthetic construct
  source 1..21: mol_type = other DNA
  misc_feature 1..19: note = RNA
  misc_feature 20..21: note = DNA
agggttgttgctgaaccgctt

SEQ ID NO: 748 - DNA, 21 bp, synthetic construct
  source 1..21: mol_type = other DNA
  misc_feature 1..19: note = RNA
  misc_feature 20..21: note = DNA
ggttcagcaacaaccctgctt

SEQ ID NO: 749 - DNA, 21 bp, synthetic construct
  source 1..21: mol_type = other DNA
  misc_feature 1..19: note = RNA
  misc_feature 20..21: note = DNA
gcagggttgttgctgaacctt

SEQ ID NO: 750 - DNA, 21 bp, synthetic construct
  source 1..21: mol_type = other DNA
  misc_feature 1..19: note = RNA
  misc_feature 20..21: note = DNA
gcaacaaccctgccctgtgtt

SEQ ID NO: 751 - DNA, 21 bp, synthetic construct
  source 1..21: mol_type = other DNA
  misc_feature 1..19: note = RNA
  misc_feature 20..21: note = DNA
cacagggcagggttgttgctt

SEQ ID NO: 752 - DNA, 21 bp, synthetic construct
  source 1..21: mol_type = other DNA
  misc_feature 1..19: note = RNA
  misc_feature 20..21: note = DNA
aacaaccctgccctgtgcatt

SEQ ID NO: 753 - DNA, 21 bp, synthetic construct
  source 1..21: mol_type = other DNA
  misc_feature 1..19: note = RNA
  misc_feature 20..21: note = DNA
tgcacagggcagggttgtttt

SEQ ID NO: 754 - DNA, 21 bp, synthetic construct
  source 1..21: mol_type = other DNA
  misc_feature 1..19: note = RNA
  misc_feature 20..21: note = DNA
acgtggagagcatccagtgtt

SEQ ID NO: 755 - DNA, 21 bp, synthetic construct
  source 1..21: mol_type = other DNA
  misc_feature 1..19: note = RNA
  misc_feature 20..21: note = DNA
cactggatgctctccacgttt

SEQ ID NO: 756 - DNA, 21 bp, synthetic construct
  source 1..21: mol_type = other DNA
  misc_feature 1..19: note = RNA
  misc_feature 20..21: note = DNA
cgtggagagcatccagtggtt

SEQ ID NO: 757 - DNA, 21 bp, synthetic construct
  source 1..21: mol_type = other DNA
  misc_feature 1..19: note = RNA
  misc_feature 20..21: note = DNA
ccactggatgctctccacgtt

SEQ ID NO: 758 - DNA, 21 bp, synthetic construct
  source 1..21: mol_type = other DNA
  misc_feature 1..19: note = RNA
  misc_feature 20..21: note = DNA
gtggagagcatccagtggctt

SEQ ID NO: 759 - DNA, 21 bp, synthetic construct
  source 1..21: mol_type = other DNA
  misc_feature 1..19: note = RNA
  misc_feature 20..21: note = DNA
gccactggatgctctccactt

SEQ ID NO: 760 - DNA, 21 bp, synthetic construct
  source 1..21: mol_type = other DNA
  misc_feature 1..19: note = RNA
  misc_feature 20..21: note = DNA
gagagcatccagtggcgggtt

SEQ ID NO: 761 - DNA, 21 bp, synthetic construct
  source 1..21: mol_type = other DNA
  misc_feature 1..19: note = RNA
  misc_feature 20..21: note = DNA
cccgccactggatgctctctt

SEQ ID NO: 762 - DNA, 21 bp, synthetic construct
  source 1..21: mol_type = other DNA
  misc_feature 1..19: note = RNA
  misc_feature 20..21: note = DNA
agagcatccagtggcgggatt

SEQ ID NO: 763 - DNA, 21 bp, synthetic construct
  source 1..21: mol_type = other DNA
  misc_feature 1..19: note = RNA
  misc_feature 20..21: note = DNA
tcccgccactggatgctcttt

SEQ ID NO: 764 - DNA, 21 bp, synthetic construct
  source 1..21: mol_type = other DNA
  misc_feature 1..19: note = RNA
  misc_feature 20..21: note = DNA
gagcatccagtggcgggactt

SEQ ID NO: 765 - DNA, 21 bp, synthetic construct
  source 1..21: mol_type = other DNA
  misc_feature 1..19: note = RNA
  misc_feature 20..21: note = DNA
gtcccgccactggatgctctt

SEQ ID NO: 766 - DNA, 21 bp, synthetic construct
  source 1..21: mol_type = other DNA
  misc_feature 1..19: note = RNA
  misc_feature 20..21: note = DNA
cagtggcgggacatagtcatt

SEQ ID NO: 767 - DNA, 21 bp, synthetic construct
  source 1..21: mol_type = other DNA
  misc_feature 1..19: note = RNA
  misc_feature 20..21: note = DNA
tgactatgtcccgccactgtt

SEQ ID NO: 768 - DNA, 21 bp, synthetic construct
  source 1..21: mol_type = other DNA
  misc_feature 1..19: note = RNA
  misc_feature 20..21: note = DNA
agtggcgggacatagtcagtt

SEQ ID NO: 769 - DNA, 21 bp, synthetic construct
  source 1..21: mol_type = other DNA
  misc_feature 1..19: note = RNA
  misc_feature 20..21: note = DNA
ctgactatgtcccgccacttt

SEQ ID NO: 770 - DNA, 21 bp, synthetic construct
  source 1..21: mol_type = other DNA
  misc_feature 1..19: note = RNA
  misc_feature 20..21: note = DNA
tggcgggacatagtcagcatt

SEQ ID NO: 771 - DNA, 21 bp, synthetic construct
  source 1..21: mol_type = other DNA
  misc_feature 1..19: note = RNA
  misc_feature 20..21: note = DNA
tgctgactatgtcccgccatt

SEQ ID NO: 772 - DNA, 21 bp, synthetic construct
  source 1..21: mol_type = other DNA
  misc_feature 1..19: note = RNA
  misc_feature 20..21: note = DNA
ggcgggacatagtcagcagtt

SEQ ID NO: 773 - DNA, 21 bp, synthetic construct
  source 1..21: mol_type = other DNA
  misc_feature 1..19: note = RNA
  misc_feature 20..21: note = DNA
ctgctgactatgtcccgcctt

SEQ ID NO: 774 - DNA, 21 bp, synthetic construct
  source 1..21: mol_type = other DNA
  misc_feature 1..19: note = RNA
  misc_feature 20..21: note = DNA
ctcagcaacatgtcgatggtt

SEQ ID NO: 775 - DNA, 21 bp, synthetic construct
  source 1..21: mol_type = other DNA
  misc_feature 1..19: note = RNA
  misc_feature 20..21: note = DNA
ccatcgacatgttgctgagtt

SEQ ID NO: 776 - DNA, 21 bp, synthetic construct
  source 1..21: mol_type = other DNA
  misc_feature 1..19: note = RNA
  misc_feature 20..21: note = DNA
cagcaacatgtcgatggactt

SEQ ID NO: 777 - DNA, 21 bp, synthetic construct
  source 1..21: mol_type = other DNA
  misc_feature 1..19: note = RNA
  misc_feature 20..21: note = DNA
gtccatcgacatgttgctgtt

SEQ ID NO: 778 - DNA, 21 bp, synthetic construct
  source 1..21: mol_type = other DNA
  misc_feature 1..19: note = RNA
  misc_feature 20..21: note = DNA
agcaacatgtcgatggacttt

SEQ ID NO: 779 - DNA, 21 bp, synthetic construct
  source 1..21: mol_type = other DNA
  misc_feature 1..19: note = RNA
  misc_feature 20..21: note = DNA
agtccatcgacatgttgcttt

SEQ ID NO: 780 - DNA, 21 bp, synthetic construct
  source 1..21: mol_type = other DNA
  misc_feature 1..19: note = RNA
  misc_feature 20..21: note = DNA
gcaacatgtcgatggactttt

SEQ ID NO: 781 - DNA, 21 bp, synthetic construct
  source 1..21: mol_type = other DNA
  misc_feature 1..19: note = RNA
  misc_feature 20..21: note = DNA
aagtccatcgacatgttgctt

SEQ ID NO: 782 - DNA, 21 bp, synthetic construct
  source 1..21: mol_type = other DNA
  misc_feature 1..19: note = RNA
  misc_feature 20..21: note = DNA
aacatgtcgatggacttcctt

SEQ ID NO: 783 - DNA, 21 bp, synthetic construct
  source 1..21: mol_type = other DNA
  misc_feature 1..19: note = RNA
  misc_feature 20..21: note = DNA
ggaagtccatcgacatgtttt

SEQ ID NO: 784 - DNA, 21 bp, synthetic construct
  source 1..21: mol_type = other DNA
  misc_feature 1..19: note = RNA
  misc_feature 20..21: note = DNA
acatgtcgatggacttccatt

SEQ ID NO: 785 - DNA, 21 bp, synthetic construct
  source 1..21: mol_type = other DNA
  misc_feature 1..19: note = RNA
  misc_feature 20..21: note = DNA
tggaagtccatcgacatgttt

SEQ ID NO: 786 - DNA, 21 bp, synthetic construct
  source 1..21: mol_type = other DNA
  misc_feature 1..19: note = RNA
  misc_feature 20..21: note = DNA
catgtcgatggacttccagtt

SEQ ID NO: 787 - DNA, 21 bp, synthetic construct
  source 1..21: mol_type = other DNA
  misc_feature 1..19: note = RNA
  misc_feature 20..21: note = DNA
ctggaagtccatcgacatgtt

SEQ ID NO: 788 - DNA, 21 bp, synthetic construct
  source 1..21: mol_type = other DNA
  misc_feature 1..19: note = RNA
  misc_feature 20..21: note = DNA
atgtcgatggacttccagatt

SEQ ID NO: 789 - DNA, 21 bp, synthetic construct
  source 1..21: mol_type = other DNA
  misc_feature 1..19: note = RNA
  misc_feature 20..21: note = DNA
tctggaagtccatcgacattt

SEQ ID NO: 790 - DNA, 21 bp, synthetic construct
  source 1..21: mol_type = other DNA
  misc_feature 1..19: note = RNA
  misc_feature 20..21: note = DNA
tcgatggacttccagaacctt

SEQ ID NO: 791 - DNA, 21 bp, synthetic construct
  source 1..21: mol_type = other DNA
  misc_feature 1..19: note = RNA
  misc_feature 20..21: note = DNA
ggttctggaagtccatcgatt

SEQ ID NO: 792 - DNA, 21 bp, synthetic construct
  source 1..21: mol_type = other DNA
  misc_feature 1..19: note = RNA
  misc_feature 20..21: note = DNA
gaaccacctgggcagctgctt

SEQ ID NO: 793 - DNA, 21 bp, synthetic construct
  source 1..21: mol_type = other DNA
  misc_feature 1..19: note = RNA
  misc_feature 20..21: note = DNA
gcagctgcccaggtggttctt

SEQ ID NO: 794 - DNA, 21 bp, synthetic construct
  source 1..21: mol_type = other DNA
  misc_feature 1..19: note = RNA
  misc_feature 20..21: note = DNA
gggcagctgccaaaagtgttt

SEQ ID NO: 795 - DNA, 21 bp, synthetic construct
  source 1..21: mol_type = other DNA
  misc_feature 1..19: note = RNA
  misc_feature 20..21: note = DNA
acacttttggcagctgccctt

SEQ ID NO: 796 - DNA, 21 bp, synthetic construct
  source 1..21: mol_type = other DNA
  misc_feature 1..19: note = RNA
  misc_feature 20..21: note = DNA
gcagctgccaaaagtgtgatt

SEQ ID NO: 797 - DNA, 21 bp, synthetic construct
  source 1..21: mol_type = other DNA
  misc_feature 1..19: note = RNA
  misc_feature 20..21: note = DNA
tcacacttttggcagctgctt

SEQ ID NO: 798 - DNA, 21 bp, synthetic construct
  source 1..21: mol_type = other DNA
  misc_feature 1..19: note = RNA
  misc_feature 20..21: note = DNA
tgccaaaagtgtgatccaatt

SEQ ID NO: 799 - DNA, 21 bp, synthetic construct
  source 1..21: mol_type = other DNA
  misc_feature 1..19: note = RNA
  misc_feature 20..21: note = DNA
ttggatcacacttttggcatt

SEQ ID NO: 800 - DNA, 21 bp, synthetic construct
  source 1..21: mol_type = other DNA
  misc_feature 1..19: note = RNA
  misc_feature 20..21: note = DNA
gccaaaagtgtgatccaagtt

SEQ ID NO: 801 - DNA, 21 bp, synthetic construct
  source 1..21: mol_type = other DNA
  misc_feature 1..19: note = RNA
  misc_feature 20..21: note = DNA
cttggatcacacttttggctt

SEQ ID NO: 802 - DNA, 21 bp, synthetic construct
  source 1..21: mol_type = other DNA
  misc_feature 1..19: note = RNA
  misc_feature 20..21: note = DNA
ccaaaagtgtgatccaagctt

SEQ ID NO: 803 - DNA, 21 bp, synthetic construct
  source 1..21: mol_type = other DNA
  misc_feature 1..19: note = RNA
  misc_feature 20..21: note = DNA
gcttggatcacacttttggtt

SEQ ID NO: 804 - DNA, 21 bp, synthetic construct
  source 1..21: mol_type = other DNA
  misc_feature 1..19: note = RNA
  misc_feature 20..21: note = DNA
aaagtgtgatccaagctgttt

SEQ ID NO: 805 - DNA, 21 bp, synthetic construct
  source 1..21: mol_type = other DNA
  misc_feature 1..19: note = RNA
  misc_feature 20..21: note = DNA
acagcttggatcacacttttt

SEQ ID NO: 806 - DNA, 21 bp, synthetic construct
  source 1..21: mol_type = other DNA
  misc_feature 1..19: note = RNA
  misc_feature 20..21: note = DNA
tgtgatccaagctgtcccatt

SEQ ID NO: 807 - DNA, 21 bp, synthetic construct
  source 1..21: mol_type = other DNA
  misc_feature 1..19: note = RNA
  misc_feature 20..21: note = DNA
tgggacagcttggatcacatt

SEQ ID NO: 808 - DNA, 21 bp, synthetic construct
  source 1..21: mol_type = other DNA
  misc_feature 1..19: note = RNA
  misc_feature 20..21: note = DNA
gtgatccaagctgtcccaatt

SEQ ID NO: 809 - DNA, 21 bp, synthetic construct
  source 1..21: mol_type = other DNA
  misc_feature 1..19: note = RNA
  misc_feature 20..21: note = DNA
ttgggacagcttggatcactt

SEQ ID NO: 810 - DNA, 21 bp, synthetic construct
  source 1..21: mol_type = other DNA
  misc_feature 1..19: note = RNA
  misc_feature 20..21: note = DNA
gatccaagctgtcccaatgtt

SEQ ID NO: 811 - DNA, 21 bp, synthetic construct
  source 1..21: mol_type = other DNA
  misc_feature 1..19: note = RNA
  misc_feature 20..21: note = DNA
cattgggacagcttggatctt

SEQ ID NO: 812 - DNA, 21 bp, synthetic construct
  source 1..21: mol_type = other DNA
  misc_feature 1..19: note = RNA
  misc_feature 20..21: note = DNA
atccaagctgtcccaatggtt

SEQ ID NO: 813 - DNA, 21 bp, synthetic construct
  source 1..21: mol_type = other DNA
  misc_feature 1..19: note = RNA
  misc_feature 20..21: note = DNA
ccattgggacagcttggattt

SEQ ID NO: 814 - DNA, 21 bp, synthetic construct
  source 1..21: mol_type = other DNA
  misc_feature 1..19: note = RNA
  misc_feature 20..21: note = DNA
tccaagctgtcccaatgggtt

SEQ ID NO: 815 - DNA, 21 bp, synthetic construct
  source 1..21: mol_type = other DNA
  misc_feature 1..19: note = RNA
  misc_feature 20..21: note = DNA
cccattgggacagcttggatt

SEQ ID NO: 816 - DNA, 21 bp, synthetic construct
  source 1..21: mol_type = other DNA
  misc_feature 1..19: note = RNA
  misc_feature 20..21: note = DNA
ccaagctgtcccaatgggatt

SEQ ID NO: 817 - DNA, 21 bp, synthetic construct
  source 1..21: mol_type = other DNA
  misc_feature 1..19: note = RNA
  misc_feature 20..21: note = DNA
tcccattgggacagcttggtt

SEQ ID NO: 818 - DNA, 21 bp, synthetic construct
  source 1..21: mol_type = other DNA
  misc_feature 1..19: note = RNA
  misc_feature 20..21: note = DNA
caagctgtcccaatgggagtt

SEQ ID NO: 819 - DNA, 21 bp, synthetic construct
  source 1..21: mol_type = other DNA
  misc_feature 1..19: note = RNA
  misc_feature 20..21: note = DNA
ctcccattgggacagcttgtt

SEQ ID NO: 820 - DNA, 21 bp, synthetic construct
  source 1..21: mol_type = other DNA
  misc_feature 1..19: note = RNA
  misc_feature 20..21: note = DNA
agctgtcccaatgggagcttt

SEQ ID NO: 821 - DNA, 21 bp, synthetic construct
  source 1..21: mol_type = other DNA
  misc_feature 1..19: note = RNA
  misc_feature 20..21: note = DNA
agctcccattgggacagcttt

SEQ ID NO: 822 - DNA, 21 bp, synthetic construct
  source 1..21: mol_type = other DNA
  misc_feature 1..19: note = RNA
  misc_feature 20..21: note = DNA
tgtcccaatgggagctgcttt

SEQ ID NO: 823 - DNA, 21 bp, synthetic construct
  source 1..21: mol_type = other DNA
  misc_feature 1..19: note = RNA
  misc_feature 20..21: note = DNA
agcagctcccattgggacatt

SEQ ID NO: 824 - DNA, 21 bp, synthetic construct
  source 1..21: mol_type = other DNA
  misc_feature 1..19: note = RNA
  misc_feature 20..21: note = DNA
ggtgcaggagaggagaacttt

SEQ ID NO: 825 - DNA, 21 bp, synthetic construct
  source 1..21: mol_type = other DNA
  misc_feature 1..19: note = RNA
  misc_feature 20..21: note = DNA
agttctcctctcctgcacctt

SEQ ID NO: 826 - DNA, 21 bp, synthetic construct
  source 1..21: mol_type = other DNA
  misc_feature 1..19: note = RNA
  misc_feature 20..21: note = DNA
aaactgaccaaaatcatcttt

SEQ ID NO: 827 - DNA, 21 bp, synthetic construct
  source 1..21: mol_type = other DNA
  misc_feature 1..19: note = RNA
  misc_feature 20..21: note = DNA
agatgattttggtcagttttt

SEQ ID NO: 828 - DNA, 21 bp, synthetic construct
  source 1..21: mol_type = other DNA
  misc_feature 1..19: note = RNA
  misc_feature 20..21: note = DNA
aactgaccaaaatcatctgtt

SEQ ID NO: 829 - DNA, 21 bp, synthetic construct
  source 1..21: mol_type = other DNA
  misc_feature 1..19: note = RNA
  misc_feature 20..21: note = DNA
cagatgattttggtcagtttt

SEQ ID NO: 830 - DNA, 21 bp, synthetic construct
  source 1..21: mol_type = other DNA
  misc_feature 1..19: note = RNA
  misc_feature 20..21: note = DNA
actgaccaaaatcatctgttt

SEQ ID NO: 831 - DNA, 21 bp, synthetic construct
  source 1..21: mol_type = other DNA
  misc_feature 1..19: note = RNA
  misc_feature 20..21: note = DNA
acagatgattttggtcagttt

SEQ ID NO: 832 - DNA, 21 bp, synthetic construct
  source 1..21: mol_type = other DNA
  misc_feature 1..19: note = RNA
  misc_feature 20..21: note = DNA
accaaaatcatctgtgccctt

SEQ ID NO: 833 - DNA, 21 bp, synthetic construct
  source 1..21: mol_type = other DNA
  misc_feature 1..19: note = RNA
  misc_feature 20..21: note = DNA
gggcacagatgattttggttt

SEQ ID NO: 834 - DNA, 21 bp, synthetic construct
  source 1..21: mol_type = other DNA
  misc_feature 1..19: note = RNA
  misc_feature 20..21: note = DNA
ccaaaatcatctgtgcccatt

SEQ ID NO: 835 - DNA, 21 bp, synthetic construct
  source 1..21: mol_type = other DNA
  misc_feature 1..19: note = RNA
  misc_feature 20..21: note = DNA
tgggcacagatgattttggtt

SEQ ID NO: 836 - DNA, 21 bp, synthetic construct
  source 1..21: mol_type = other DNA
  misc_feature 1..19: note = RNA
  misc_feature 20..21: note = DNA
aaatcatctgtgcccagcatt

SEQ ID NO: 837 - DNA, 21 bp, synthetic construct
  source 1..21: mol_type = other DNA
  misc_feature 1..19: note = RNA
  misc_feature 20..21: note = DNA
tgctgggcacagatgattttt

SEQ ID NO: 838 - DNA, 21 bp, synthetic construct
  source 1..21: mol_type = other DNA
  misc_feature 1..19: note = RNA
  misc_feature 20..21: note = DNA
gtgcccagcagtgctccggtt

SEQ ID NO: 839 - DNA, 21 bp, synthetic construct
  source 1..21: mol_type = other DNA
  misc_feature 1..19: note = RNA
  misc_feature 20..21: note = DNA
ccggagcactgctgggcactt

SEQ ID NO: 840 - DNA, 21 bp, synthetic construct
  source 1..21: mol_type = other DNA
  misc_feature 1..19: note = RNA
  misc_feature 20..21: note = DNA
gcccagcagtgctccgggctt

SEQ ID NO: 841 - DNA, 21 bp, synthetic construct
  source 1..21: mol_type = other DNA
  misc_feature 1..19: note = RNA
  misc_feature 20..21: note = DNA
gcccggagcactgctgggctt

SEQ ID NO: 842 - DNA, 21 bp, synthetic construct
  source 1..21: mol_type = other DNA
  misc_feature 1..19: note = RNA
  misc_feature 20..21: note = DNA
ccagtgactgctgccacaatt

SEQ ID NO: 843 - DNA, 21 bp, synthetic construct
  source 1..21: mol_type = other DNA
  misc_feature 1..19: note = RNA
  misc_feature 20..21: note = DNA
ttgtggcagcagtcactggtt

SEQ ID NO: 844 - DNA, 21 bp, synthetic construct
  source 1..21: mol_type = other DNA
  misc_feature 1..19: note = RNA
  misc_feature 20..21: note = DNA
cagtgactgctgccacaactt

SEQ ID NO: 845 - DNA, 21 bp, synthetic construct
  source 1..21: mol_type = other DNA
  misc_feature 1..19: note = RNA
  misc_feature 20..21: note = DNA
gttgtggcagcagtcactgtt

SEQ ID NO: 846 - DNA, 21 bp, synthetic construct
  source 1..21: mol_type = other DNA
  misc_feature 1..19: note = RNA
  misc_feature 20..21: note = DNA
gagagcgactgcctggtcttt

SEQ ID NO: 847 - DNA, 21 bp, synthetic construct
  source 1..21: mol_type = other DNA
  misc_feature 1..19: note = RNA
  misc_feature 20..21: note = DNA
agaccaggcagtcgctctctt

SEQ ID NO: 848 - DNA, 21 bp, synthetic construct
  source 1..21: mol_type = other DNA
  misc_feature 1..19: note = RNA
  misc_feature 20..21: note = DNA
agagcgactgcctggtctgtt

SEQ ID NO: 849 - DNA, 21 bp, synthetic construct
  source 1..21: mol_type = other DNA
  misc_feature 1..19: note = RNA
  misc_feature 20..21: note = DNA
cagaccaggcagtcgctcttt

SEQ ID NO: 850 - DNA, 21 bp, synthetic construct
  source 1..21: mol_type = other DNA
  misc_feature 1..19: note = RNA
  misc_feature 20..21: note = DNA
gagcgactgcctggtctgctt

SEQ ID NO: 851 - DNA, 21 bp, synthetic construct
  source 1..21: mol_type = other DNA
  misc_feature 1..19: note = RNA
  misc_feature 20..21: note = DNA
gcagaccaggcagtcgctctt

SEQ ID NO: 852 - DNA, 21 bp, synthetic construct
  source 1..21: mol_type = other DNA
  misc_feature 1..19: note = RNA
  misc_feature 20..21: note = DNA
agcgactgcctggtctgcctt

SEQ ID NO: 853 - DNA, 21 bp, synthetic construct
  source 1..21: mol_type = other DNA
  misc_feature 1..19: note = RNA
  misc_feature 20..21: note = DNA
ggcagaccaggcagtcgcttt

SEQ ID NO: 854 - DNA, 21 bp, synthetic construct
  source 1..21: mol_type = other DNA
  misc_feature 1..19: note = RNA
  misc_feature 20..21: note = DNA
gcgactgcctggtctgccgtt

SEQ ID NO: 855 - DNA, 21 bp, synthetic construct
  source 1..21: mol_type = other DNA
  misc_feature 1..19: note = RNA
  misc_feature 20..21: note = DNA
cggcagaccaggcagtcgctt

SEQ ID NO: 856 - DNA, 21 bp, synthetic construct
  source 1..21: mol_type = other DNA
  misc_feature 1..19: note = RNA
  misc_feature 20..21: note = DNA
cgactgcctggtctgccgctt

SEQ ID NO: 857 - DNA, 21 bp, synthetic construct
  source 1..21: mol_type = other DNA
  misc_feature 1..19: note = RNA
  misc_feature 20..21: note = DNA
gcggcagaccaggcagtcgtt

SEQ ID NO: 858 - DNA, 21 bp, synthetic construct
  source 1..21: mol_type = other DNA
  misc_feature 1..19: note = RNA
  misc_feature 20..21: note = DNA
gcctggtctgccgcaaatttt

SEQ ID NO: 859 - DNA, 21 bp, synthetic construct
  source 1..21: mol_type = other DNA
  misc_feature 1..19: note = RNA
  misc_feature 20..21: note = DNA
aatttgcggcagaccaggctt

SEQ ID NO: 860 - DNA, 21 bp, synthetic construct
  source 1..21: mol_type = other DNA
  misc_feature 1..19: note = RNA
  misc_feature 20..21: note = DNA
cctggtctgccgcaaattctt

SEQ ID NO: 861 - DNA, 21 bp, synthetic construct
  source 1..21: mol_type = other DNA
  misc_feature 1..19: note = RNA
  misc_feature 20..21: note = DNA
gaatttgcggcagaccaggtt

SEQ ID NO: 862 - DNA, 21 bp, synthetic construct
  source 1..21: mol_type = other DNA
  misc_feature 1..19: note = RNA
  misc_feature 20..21: note = DNA
ctggtctgccgcaaattcctt

SEQ ID NO: 863 - DNA, 21 bp, synthetic construct
  source 1..21: mol_type = other DNA
  misc_feature 1..19: note = RNA
  misc_feature 20..21: note = DNA
ggaatttgcggcagaccagtt

SEQ ID NO: 864 - DNA, 21 bp, synthetic construct
  source 1..21: mol_type = other DNA
  misc_feature 1..19: note = RNA
  misc_feature 20..21: note = DNA
tggtctgccgcaaattccgtt

SEQ ID NO: 865 - DNA, 21 bp, synthetic construct
  source 1..21: mol_type = other DNA
  misc_feature 1..19: note = RNA
  misc_feature 20..21: note = DNA
cggaatttgcggcagaccatt

SEQ ID NO: 866 - DNA, 21 bp, synthetic construct
  source 1..21: mol_type = other DNA
  misc_feature 1..19: note = RNA
  misc_feature 20..21: note = DNA
gtctgccgcaaattccgagtt

SEQ ID NO: 867 - DNA, 21 bp, synthetic construct
  source 1..21: mol_type = other DNA
  misc_feature 1..19: note = RNA
  misc_feature 20..21: note = DNA
ctcggaatttgcggcagactt

SEQ ID NO: 868 - DNA, 21 bp, synthetic construct
  source 1..21: mol_type = other DNA
  misc_feature 1..19: note = RNA
  misc_feature 20..21: note = DNA
tctgccgcaaattccgagatt

SEQ ID NO: 869 - DNA, 21 bp, synthetic construct
  source 1..21: mol_type = other DNA
  misc_feature 1..19: note = RNA
  misc_feature 20..21: note = DNA
tctcggaatttgcggcagatt

SEQ ID NO: 870 - DNA, 21 bp, synthetic construct
  source 1..21: mol_type = other DNA
  misc_feature 1..19: note = RNA
  misc_feature 20..21: note = DNA
ctgccgcaaattccgagactt

SEQ ID NO: 871 - DNA, 21 bp, synthetic construct
  source 1..21: mol_type = other DNA
  misc_feature 1..19: note = RNA
  misc_feature 20..21: note = DNA
gtctcggaatttgcggcagtt

SEQ ID NO: 872 - DNA, 21 bp, synthetic construct
  source 1..21: mol_type = other DNA
  misc_feature 1..19: note = RNA
  misc_feature 20..21: note = DNA
tgccgcaaattccgagacgtt

SEQ ID NO: 873 - DNA, 21 bp, synthetic construct
  source 1..21: mol_type = other DNA
  misc_feature 1..19: note = RNA
  misc_feature 20..21: note = DNA
cgtctcggaatttgcggcatt

SEQ ID NO: 874 - DNA, 21 bp, synthetic construct
  source 1..21: mol_type = other DNA
  misc_feature 1..19: note = RNA
  misc_feature 20..21: note = DNA
ccgcaaattccgagacgaatt

SEQ ID NO: 875 - DNA, 21 bp, synthetic construct
  source 1..21: mol_type = other DNA
  misc_feature 1..19: note = RNA
  misc_feature 20..21: note = DNA
ttcgtctcggaatttgcggtt

SEQ ID NO: 876 - DNA, 21 bp, synthetic construct
  source 1..21: mol_type = other DNA
  misc_feature 1..19: note = RNA
  misc_feature 20..21: note = DNA
aattccgagacgaagccactt

SEQ ID NO: 877 - DNA, 21 bp, synthetic construct
  source 1..21: mol_type = other DNA
  misc_feature 1..19: note = RNA
  misc_feature 20..21: note = DNA
gtggcttcgtctcggaatttt

SEQ ID NO: 878 - DNA, 21 bp, synthetic construct
  source 1..21: mol_type = other DNA
  misc_feature 1..19: note = RNA
  misc_feature 20..21: note = DNA
attccgagacgaagccacgtt

SEQ ID NO: 879 - DNA, 21 bp, synthetic construct
  source 1..21: mol_type = other DNA
  misc_feature 1..19: note = RNA
  misc_feature 20..21: note = DNA
cgtggcttcgtctcggaattt

SEQ ID NO: 880 - DNA, 21 bp, synthetic construct
  source 1..21: mol_type = other DNA
  misc_feature 1..19: note = RNA
  misc_feature 20..21: note = DNA
ttccgagacgaagccacgttt

SEQ ID NO: 881 - DNA, 21 bp, synthetic construct
  source 1..21: mol_type = other DNA
  misc_feature 1..19: note = RNA
  misc_feature 20..21: note = DNA
acgtggcttcgtctcggaatt

SEQ ID NO: 882 - DNA, 21 bp, synthetic construct
  source 1..21: mol_type = other DNA
  misc_feature 1..19: note = RNA
  misc_feature 20..21: note = DNA
tccgagacgaagccacgtgtt

SEQ ID NO: 883 - DNA, 21 bp, synthetic construct
  source 1..21: mol_type = other DNA
  misc_feature 1..19: note = RNA
  misc_feature 20..21: note = DNA
cacgtggcttcgtctcggatt

SEQ ID NO: 884 - DNA, 21 bp, synthetic construct
  source 1..21: mol_type = other DNA
  misc_feature 1..19: note = RNA
  misc_feature 20..21: note = DNA
ccgagacgaagccacgtgctt

SEQ ID NO: 885 - DNA, 21 bp, synthetic construct
  source 1..21: mol_type = other DNA
  misc_feature 1..19: note = RNA
  misc_feature 20..21: note = DNA
gcacgtggcttcgtctcggtt

SEQ ID NO: 886 - DNA, 21 bp, synthetic construct
  source 1..21: mol_type = other DNA
  misc_feature 1..19: note = RNA
  misc_feature 20..21: note = DNA
gagacgaagccacgtgcaatt

SEQ ID NO: 887 - DNA, 21 bp, synthetic construct
  source 1..21: mol_type = other DNA
  misc_feature 1..19: note = RNA
  misc_feature 20..21: note = DNA
ttgcacgtggcttcgtctctt

SEQ ID NO: 888 - DNA, 21 bp, synthetic construct
  source 1..21: mol_type = other DNA
  misc_feature 1..19: note = RNA
  misc_feature 20..21: note = DNA
acgaagccacgtgcaaggatt

SEQ ID NO: 889 - DNA, 21 bp, synthetic construct
  source 1..21: mol_type = other DNA
  misc_feature 1..19: note = RNA
  misc_feature 20..21: note = DNA
tccttgcacgtggcttcgttt

SEQ ID NO: 890 - DNA, 21 bp, synthetic construct
  source 1..21: mol_type = other DNA
  misc_feature 1..19: note = RNA
  misc_feature 20..21: note = DNA
cgaagccacgtgcaaggactt

SEQ ID NO: 891 - DNA, 21 bp, synthetic construct
  source 1..21: mol_type = other DNA
  misc_feature 1..19: note = RNA
  misc_feature 20..21: note = DNA
gtccttgcacgtggcttcgtt

SEQ ID NO: 892 - DNA, 21 bp, synthetic construct
  source 1..21: mol_type = other DNA
  misc_feature 1..19: note = RNA
  misc_feature 20..21: note = DNA
gaagccacgtgcaaggacatt

SEQ ID NO: 893 - DNA, 21 bp, synthetic construct
  source 1..21: mol_type = other DNA
  misc_feature 1..19: note = RNA
  misc_feature 20..21: note = DNA
tgtccttgcacgtggcttctt

SEQ ID NO: 894 - DNA, 21 bp, synthetic construct
  source 1..21: mol_type = other DNA
  misc_feature 1..19: note = RNA
  misc_feature 20..21: note = DNA
aagccacgtgcaaggacactt

SEQ ID NO: 895 - DNA, 21 bp, synthetic construct
  source 1..21: mol_type = other DNA
  misc_feature 1..19: note = RNA
  misc_feature 20..21: note = DNA
gtgtccttgcacgtggctttt

SEQ ID NO: 896 - DNA, 21 bp, synthetic construct
  source 1..21: mol_type = other DNA
  misc_feature 1..19: note = RNA
  misc_feature 20..21: note = DNA
ccccactcatgctctacaatt

SEQ ID NO: 897 - DNA, 21 bp, synthetic construct
  source 1..21: mol_type = other DNA
  misc_feature 1..19: note = RNA
  misc_feature 20..21: note = DNA
ttgtagagcatgagtggggtt

SEQ ID NO: 898 - DNA, 21 bp, synthetic construct
  source 1..21: mol_type = other DNA
  misc_feature 1..19: note = RNA
  misc_feature 20..21: note = DNA
ccactcatgctctacaacctt

SEQ ID NO: 899 - DNA, 21 bp, synthetic construct
  source 1..21: mol_type = other DNA
  misc_feature 1..19: note = RNA
  misc_feature 20..21: note = DNA
ggttgtagagcatgagtggtt

SEQ ID NO: 900 - DNA, 21 bp, synthetic construct
  source 1..21: mol_type = other DNA
  misc_feature 1..19: note = RNA
  misc_feature 20..21: note = DNA
actcatgctctacaacccctt

SEQ ID NO: 901 - DNA, 21 bp, synthetic construct
  source 1..21: mol_type = other DNA
  misc_feature 1..19: note = RNA
  misc_feature 20..21: note = DNA
ggggttgtagagcatgagttt

SEQ ID NO: 902 - DNA, 21 bp, synthetic construct
  source 1..21: mol_type = other DNA
  misc_feature 1..19: note = RNA
  misc_feature 20..21: note = DNA
catgctctacaaccccacctt

SEQ ID NO: 903 - DNA, 21 bp, synthetic construct
  source 1..21: mol_type = other DNA
  misc_feature 1..19: note = RNA
  misc_feature 20..21: note = DNA
ggtggggttgtagagcatgtt

SEQ ID NO: 904 - DNA, 21 bp, synthetic construct
  source 1..21: mol_type = other DNA
  misc_feature 1..19: note = RNA
  misc_feature 20..21: note = DNA
ccagatggatgtgaacccctt

SEQ ID NO: 905 - DNA, 21 bp, synthetic construct
  source 1..21: mol_type = other DNA
  misc_feature 1..19: note = RNA
  misc_feature 20..21: note = DNA
ggggttcacatccatctggtt

SEQ ID NO: 906 - DNA, 21 bp, synthetic construct
  source 1..21: mol_type = other DNA
  misc_feature 1..19: note = RNA
  misc_feature 20..21: note = DNA
agatggatgtgaaccccgatt

SEQ ID NO: 907 - DNA, 21 bp, synthetic construct
  source 1..21: mol_type = other DNA
  misc_feature 1..19: note = RNA
  misc_feature 20..21: note = DNA
tcggggttcacatccatcttt

SEQ ID NO: 908 - DNA, 21 bp, synthetic construct
  source 1..21: mol_type = other DNA
  misc_feature 1..19: note = RNA
  misc_feature 20..21: note = DNA
gatggatgtgaaccccgagtt

SEQ ID NO: 909 - DNA, 21 bp, synthetic construct
  source 1..21: mol_type = other DNA
  misc_feature 1..19: note = RNA
  misc_feature 20..21: note = DNA
ctcggggttcacatccatctt

SEQ ID NO: 910 - DNA, 21 bp, synthetic construct
  source 1..21: mol_type = other DNA
  misc_feature 1..19: note = RNA
  misc_feature 20..21: note = DNA
atggatgtgaaccccgaggtt

SEQ ID NO: 911 - DNA, 21 bp, synthetic construct
  source 1..21: mol_type = other DNA
  misc_feature 1..19: note = RNA
  misc_feature 20..21: note = DNA
cctcggggttcacatccattt

SEQ ID NO: 912 - DNA, 21 bp, synthetic construct
  source 1..21: mol_type = other DNA
  misc_feature 1..19: note = RNA
  misc_feature 20..21: note = DNA
ggatgtgaaccccgagggctt

SEQ ID NO: 913 - DNA, 21 bp, synthetic construct
  source 1..21: mol_type = other DNA
  misc_feature 1..19: note = RNA
  misc_feature 20..21: note = DNA
gccctcggggttcacatcctt

SEQ ID NO: 914 - DNA, 21 bp, synthetic construct
  source 1..21: mol_type = other DNA
  misc_feature 1..19: note = RNA
  misc_feature 20..21: note = DNA
gatgtgaaccccgagggcatt

SEQ ID NO: 915 - DNA, 21 bp, synthetic construct
  source 1..21: mol_type = other DNA
  misc_feature 1..19: note = RNA
  misc_feature 20..21: note = DNA
tgccctcggggttcacatctt

SEQ ID NO: 916 - DNA, 21 bp, synthetic construct
  source 1..21: mol_type = other DNA
  misc_feature 1..19: note = RNA
  misc_feature 20..21: note = DNA
tgtgaaccccgagggcaaatt

SEQ ID NO: 917 - DNA, 21 bp, synthetic construct
  source 1..21: mol_type = other DNA
  misc_feature 1..19: note = RNA
  misc_feature 20..21: note = DNA
tttgccctcggggttcacatt

SEQ ID NO: 918 - DNA, 21 bp, synthetic construct
  source 1..21: mol_type = other DNA
  misc_feature 1..19: note = RNA
  misc_feature 20..21: note = DNA
aaccccgagggcaaatacatt

SEQ ID NO: 919 - DNA, 21 bp, synthetic construct
  source 1..21: mol_type = other DNA
  misc_feature 1..19: note = RNA
  misc_feature 20..21: note = DNA
tgtatttgccctcggggtttt

SEQ ID NO: 920 - DNA, 21 bp, synthetic construct
  source 1..21: mol_type = other DNA
  misc_feature 1..19: note = RNA
  misc_feature 20..21: note = DNA
accccgagggcaaatacagtt

SEQ ID NO: 921 - DNA, 21 bp, synthetic construct
  source 1..21: mol_type = other DNA
  misc_feature 1..19: note = RNA
  misc_feature 20..21: note = DNA
ctgtatttgccctcggggttt

SEQ ID NO: 922 - DNA, 21 bp, synthetic construct
  source 1..21: mol_type = other DNA
  misc_feature 1..19: note = RNA
  misc_feature 20..21: note = DNA
cccgagggcaaatacagcttt

SEQ ID NO: 923 - DNA, 21 bp, synthetic construct
  source 1..21: mol_type = other DNA
  misc_feature 1..19: note = RNA
  misc_feature 20..21: note = DNA
agctgtatttgccctcgggtt

SEQ ID NO: 924 - DNA, 21 bp, synthetic construct
  source 1..21: mol_type = other DNA
  misc_feature 1..19: note = RNA
  misc_feature 20..21: note = DNA
ccgagggcaaatacagctttt

SEQ ID NO: 925 - DNA, 21 bp, synthetic construct
  source 1..21: mol_type = other DNA
  misc_feature 1..19: note = RNA
  misc_feature 20..21: note = DNA
aagctgtatttgccctcggtt

SEQ ID NO: 926 - DNA, 21 bp, synthetic construct
  source 1..21: mol_type = other DNA
  misc_feature 1..19: note = RNA
  misc_feature 20..21: note = DNA
cgagggcaaatacagcttttt

SEQ ID NO: 927 - DNA, 21 bp, synthetic construct
  source 1..21: mol_type = other DNA
  misc_feature 1..19: note = RNA
  misc_feature 20..21: note = DNA
aaagctgtatttgccctcgtt

SEQ ID NO: 928 - DNA, 21 bp, synthetic construct
  source 1..21: mol_type = other DNA
  misc_feature 1..19: note = RNA
  misc_feature 20..21: note = DNA
aaatacagctttggtgccatt

SEQ ID NO: 929 - DNA, 21 bp, synthetic construct
  source 1..21: mol_type = other DNA
  misc_feature 1..19: note = RNA
  misc_feature 20..21: note = DNA
tggcaccaaagctgtattttt

SEQ ID NO: 930 - DNA, 21 bp, synthetic construct
  source 1..21: mol_type = other DNA
  misc_feature 1..19: note = RNA
  misc_feature 20..21: note = DNA
aatacagctttggtgccactt

SEQ ID NO: 931 - DNA, 21 bp, synthetic construct
  source 1..21: mol_type = other DNA
  misc_feature 1..19: note = RNA
  misc_feature 20..21: note = DNA
gtggcaccaaagctgtatttt

SEQ ID NO: 932 - DNA, 21 bp, synthetic construct
  source 1..21: mol_type = other DNA
  misc_feature 1..19: note = RNA
  misc_feature 20..21: note = DNA
atacagctttggtgccacctt

SEQ ID NO: 933 - DNA, 21 bp, synthetic construct
  source 1..21: mol_type = other DNA
  misc_feature 1..19: note = RNA
  misc_feature 20..21: note = DNA
ggtggcaccaaagctgtattt

SEQ ID NO: 934 - DNA, 21 bp, synthetic construct
  source 1..21: mol_type = other DNA
  misc_feature 1..19: note = RNA
  misc_feature 20..21: note = DNA
ctttggtgccacctgcgtgtt

SEQ ID NO: 935 - DNA, 21 bp, synthetic construct
  source 1..21: mol_type = other DNA
  misc_feature 1..19: note = RNA
  misc_feature 20..21: note = DNA
cacgcaggtggcaccaaagtt

SEQ ID NO: 936 - DNA, 21 bp, synthetic construct
  source 1..21: mol_type = other DNA
  misc_feature 1..19: note = RNA
  misc_feature 20..21: note = DNA
ttggtgccacctgcgtgaatt

SEQ ID NO: 937 - DNA, 21 bp, synthetic construct
  source 1..21: mol_type = other DNA
  misc_feature 1..19: note = RNA
  misc_feature 20..21: note = DNA
ttcacgcaggtggcaccaatt

SEQ ID NO: 938 - DNA, 21 bp, synthetic construct
  source 1..21: mol_type = other DNA
  misc_feature 1..19: note = RNA
  misc_feature 20..21: note = DNA
ccacctgcgtgaagaagtgtt

SEQ ID NO: 939 - DNA, 21 bp, synthetic construct
  source 1..21: mol_type = other DNA
  misc_feature 1..19: note = RNA
  misc_feature 20..21: note = DNA
cacttcttcacgcaggtggtt

SEQ ID NO: 940 - DNA, 21 bp, synthetic construct
  source 1..21: mol_type = other DNA
  misc_feature 1..19: note = RNA
  misc_feature 20..21: note = DNA
ctgcgtgaagaagtgtccctt

SEQ ID NO: 941 - DNA, 21 bp, synthetic construct
  source 1..21: mol_type = other DNA
  misc_feature 1..19: note = RNA
  misc_feature 20..21: note = DNA
gggacacttcttcacgcagtt

SEQ ID NO: 942 - DNA, 21 bp, synthetic construct
  source 1..21: mol_type = other DNA
  misc_feature 1..19: note = RNA
  misc_feature 20..21: note = DNA
tgcgtgaagaagtgtcccctt

SEQ ID NO: 943 - DNA, 21 bp, synthetic construct
  source 1..21: mol_type = other DNA
  misc_feature 1..19: note = RNA
  misc_feature 20..21: note = DNA
ggggacacttcttcacgcatt

SEQ ID NO: 944 - DNA, 21 bp, synthetic construct
  source 1..21: mol_type = other DNA
  misc_feature 1..19: note = RNA
  misc_feature 20..21: note = DNA
gcgtgaagaagtgtccccgtt

SEQ ID NO: 945 - DNA, 21 bp, synthetic construct
  source 1..21: mol_type = other DNA
  misc_feature 1..19: note = RNA
  misc_feature 20..21: note = DNA
cggggacacttcttcacgctt

SEQ ID NO: 946 - DNA, 21 bp, synthetic construct
  source 1..21: mol_type = other DNA
  misc_feature 1..19: note = RNA
  misc_feature 20..21: note = DNA
cgtgaagaagtgtccccgttt

SEQ ID NO: 947 - DNA, 21 bp, synthetic construct
  source 1..21: mol_type = other DNA
  misc_feature 1..19: note = RNA
  misc_feature 20..21: note = DNA
acggggacacttcttcacgtt

SEQ ID NO: 948 - DNA, 21 bp, synthetic construct
  source 1..21: mol_type = other DNA
  misc_feature 1..19: note = RNA
  misc_feature 20..21: note = DNA
gtgaagaagtgtccccgtatt

SEQ ID NO: 949 - DNA, 21 bp, synthetic construct
  source 1..21: mol_type = other DNA
  misc_feature 1..19: note = RNA
  misc_feature 20..21: note = DNA
tacggggacacttcttcactt

SEQ ID NO: 950 - DNA, 21 bp, synthetic construct
  source 1..21: mol_type = other DNA
  misc_feature 1..19: note = RNA
  misc_feature 20..21: note = DNA
tgaagaagtgtccccgtaatt

SEQ ID NO: 951 - DNA, 21 bp, synthetic construct
  source 1..21: mol_type = other DNA
  misc_feature 1..19: note = RNA
  misc_feature 20..21: note = DNA
ttacggggacacttcttcatt

SEQ ID NO: 952 - DNA, 21 bp, synthetic construct
  source 1..21: mol_type = other DNA
  misc_feature 1..19: note = RNA
  misc_feature 20..21: note = DNA
gaagaagtgtccccgtaattt

SEQ ID NO: 953 - DNA, 21 bp, synthetic construct
  source 1..21: mol_type = other DNA
  misc_feature 1..19: note = RNA
  misc_feature 20..21: note = DNA
attacggggacacttcttctt

SEQ ID NO: 954 - DNA, 21 bp, synthetic construct
  source 1..21: mol_type = other DNA
  misc_feature 1..19: note = RNA
  misc_feature 20..21: note = DNA
aagaagtgtccccgtaatttt

SEQ ID NO: 955 - DNA, 21 bp, synthetic construct
  source 1..21: mol_type = other DNA
  misc_feature 1..19: note = RNA
  misc_feature 20..21: note = DNA
aattacggggacacttctttt

SEQ ID NO: 956 - DNA, 21 bp, synthetic construct
  source 1..21: mol_type = other DNA
  misc_feature 1..19: note = RNA
  misc_feature 20..21: note = DNA
agaagtgtccccgtaattatt

SEQ ID NO: 957 - DNA, 21 bp, synthetic construct
  source 1..21: mol_type = other DNA
  misc_feature 1..19: note = RNA
  misc_feature 20..21: note = DNA
taattacggggacacttcttt

SEQ ID NO: 958 - DNA, 21 bp, synthetic construct
  source 1..21: mol_type = other DNA
  misc_feature 1..19: note = RNA
  misc_feature 20..21: note = DNA
gaagtgtccccgtaattattt

SEQ ID NO: 959 - DNA, 21 bp, synthetic construct
  source 1..21: mol_type = other DNA
  misc_feature 1..19: note = RNA
  misc_feature 20..21: note = DNA
ataattacggggacacttctt

SEQ ID NO: 960 - DNA, 21 bp, synthetic construct
  source 1..21: mol_type = other DNA
  misc_feature 1..19: note = RNA
  misc_feature 20..21: note = DNA
aagtgtccccgtaattatgtt

SEQ ID NO: 961 - DNA, 21 bp, synthetic construct
  source 1..21: mol_type = other DNA
  misc_feature 1..19: note = RNA
  misc_feature 20..21: note = DNA
cataattacggggacactttt

SEQ ID NO: 962 - DNA, 21 bp, synthetic construct
  source 1..21: mol_type = other DNA
  misc_feature 1..19: note = RNA
  misc_feature 20..21: note = DNA
agtgtccccgtaattatgttt

SEQ ID NO: 963 - DNA, 21 bp, synthetic construct
  source 1..21: mol_type = other DNA
  misc_feature 1..19: note = RNA
  misc_feature 20..21: note = DNA
acataattacggggacacttt

SEQ ID NO: 964 - DNA, 21 bp, synthetic construct
  source 1..21: mol_type = other DNA
  misc_feature 1..19: note = RNA
  misc_feature 20..21: note = DNA
gtgtccccgtaattatgtgtt

SEQ ID NO: 965 - DNA, 21 bp, synthetic construct
  source 1..21: mol_type = other DNA
  misc_feature 1..19: note = RNA
  misc_feature 20..21: note = DNA
cacataattacggggacactt

SEQ ID NO: 966 - DNA, 21 bp, synthetic construct
  source 1..21: mol_type = other DNA
  misc_feature 1..19: note = RNA
  misc_feature 20..21: note = DNA
tgtccccgtaattatgtggtt

SEQ ID NO: 967 - DNA, 21 bp, synthetic construct
  source 1..21: mol_type = other DNA
  misc_feature 1..19: note = RNA
  misc_feature 20..21: note = DNA
ccacataattacggggacatt

SEQ ID NO: 968 - DNA, 21 bp, synthetic construct
  source 1..21: mol_type = other DNA
  misc_feature 1..19: note = RNA
  misc_feature 20..21: note = DNA
gtccccgtaattatgtggttt

SEQ ID NO: 969 - DNA, 21 bp, synthetic construct
  source 1..21: mol_type = other DNA
  misc_feature 1..19: note = RNA
  misc_feature 20..21: note = DNA
accacataattacggggactt

SEQ ID NO: 970 - DNA, 21 bp, synthetic construct
  source 1..21: mol_type = other DNA
  misc_feature 1..19: note = RNA
  misc_feature 20..21: note = DNA
ccccgtaattatgtggtgatt

SEQ ID NO: 971 - DNA, 21 bp, synthetic construct
  source 1..21: mol_type = other DNA
  misc_feature 1..19: note = RNA
  misc_feature 20..21: note = DNA
tcaccacataattacggggtt

SEQ ID NO: 972 - DNA, 21 bp, synthetic construct
  source 1..21: mol_type = other DNA
  misc_feature 1..19: note = RNA
  misc_feature 20..21: note = DNA
ccgtaattatgtggtgacatt

SEQ ID NO: 973 - DNA, 21 bp, synthetic construct
  source 1..21: mol_type = other DNA
  misc_feature 1..19: note = RNA
  misc_feature 20..21: note = DNA
tgtcaccacataattacggtt

SEQ ID NO: 974 - DNA, 21 bp, synthetic construct
  source 1..21: mol_type = other DNA
  misc_feature 1..19: note = RNA
  misc_feature 20..21: note = DNA
gtaattatgtggtgacagatt

SEQ ID NO: 975 - DNA, 21 bp, synthetic construct
  source 1..21: mol_type = other DNA
  misc_feature 1..19: note = RNA
  misc_feature 20..21: note = DNA
tctgtcaccacataattactt

SEQ ID NO: 976 - DNA, 21 bp, synthetic construct
  source 1..21: mol_type = other DNA
  misc_feature 1..19: note = RNA
  misc_feature 20..21: note = DNA
taattatgtggtgacagattt

SEQ ID NO: 977 - DNA, 21 bp, synthetic construct
  source 1..21: mol_type = other DNA
  misc_feature 1..19: note = RNA
  misc_feature 20..21: note = DNA
atctgtcaccacataattatt

SEQ ID NO: 978 - DNA, 21 bp, synthetic construct
  source 1..21: mol_type = other DNA
  misc_feature 1..19: note = RNA
  misc_feature 20..21: note = DNA
aattatgtggtgacagatctt

SEQ ID NO: 979 - DNA, 21 bp, synthetic construct
  source 1..21: mol_type = other DNA
  misc_feature 1..19: note = RNA
  misc_feature 20..21: note = DNA
gatctgtcaccacataatttt

SEQ ID NO: 980 - DNA, 21 bp, synthetic construct
  source 1..21: mol_type = other DNA
  misc_feature 1..19: note = RNA
  misc_feature 20..21: note = DNA
attatgtggtgacagatcatt

SEQ ID NO: 981 - DNA, 21 bp, synthetic construct
  source 1..21: mol_type = other DNA
  misc_feature 1..19: note = RNA
  misc_feature 20..21: note = DNA
tgatctgtcaccacataattt

SEQ ID NO: 982 - DNA, 21 bp, synthetic construct
  source 1..21: mol_type = other DNA
  misc_feature 1..19: note = RNA
  misc_feature 20..21: note = DNA
ttatgtggtgacagatcactt

SEQ ID NO: 983 - DNA, 21 bp, synthetic construct
  source 1..21: mol_type = other DNA
  misc_feature 1..19: note = RNA
  misc_feature 20..21: note = DNA
gtgatctgtcaccacataatt

SEQ ID NO: 984 - DNA, 21 bp, synthetic construct
  source 1..21: mol_type = other DNA
  misc_feature 1..19: note = RNA
  misc_feature 20..21: note = DNA
atgtggtgacagatcacggtt

SEQ ID NO: 985 - DNA, 21 bp, synthetic construct
  source 1..21: mol_type = other DNA
  misc_feature 1..19: note = RNA
  misc_feature 20..21: note = DNA
ccgtgatctgtcaccacattt

SEQ ID NO: 986 - DNA, 21 bp, synthetic construct
  source 1..21: mol_type = other DNA
  misc_feature 1..19: note = RNA
  misc_feature 20..21: note = DNA
tggtgacagatcacggctctt

SEQ ID NO: 987 - DNA, 21 bp, synthetic construct
  source 1..21: mol_type = other DNA
  misc_feature 1..19: note = RNA
  misc_feature 20..21: note = DNA
gagccgtgatctgtcaccatt

SEQ ID NO: 988 - DNA, 21 bp, synthetic construct
  source 1..21: mol_type = other DNA
  misc_feature 1..19: note = RNA
  misc_feature 20..21: note = DNA
gtgacagatcacggctcgttt

SEQ ID NO: 989 - DNA, 21 bp, synthetic construct
  source 1..21: mol_type = other DNA
  misc_feature 1..19: note = RNA
  misc_feature 20..21: note = DNA
acgagccgtgatctgtcactt

SEQ ID NO: 990 - DNA, 21 bp, synthetic construct
  source 1..21: mol_type = other DNA
  misc_feature 1..19: note = RNA
  misc_feature 20..21: note = DNA
tgacagatcacggctcgtgtt

SEQ ID NO: 991 - DNA, 21 bp, synthetic construct
  source 1..21: mol_type = other DNA
  misc_feature 1..19: note = RNA
  misc_feature 20..21: note = DNA
cacgagccgtgatctgtcatt

SEQ ID NO: 992 - DNA, 21 bp, synthetic construct
  source 1..21: mol_type = other DNA
  misc_feature 1..19: note = RNA
  misc_feature 20..21: note = DNA
gacagatcacggctcgtgctt

SEQ ID NO: 993 - DNA, 21 bp, synthetic construct
  source 1..21: mol_type = other DNA
  misc_feature 1..19: note = RNA
  misc_feature 20..21: note = DNA
gcacgagccgtgatctgtctt

SEQ ID NO: 994 - DNA, 21 bp, synthetic construct
  source 1..21: mol_type = other DNA
  misc_feature 1..19: note = RNA
  misc_feature 20..21: note = DNA
acagatcacggctcgtgcgtt

SEQ ID NO: 995 - DNA, 21 bp, synthetic construct
  source 1..21: mol_type = other DNA
  misc_feature 1..19: note = RNA
  misc_feature 20..21: note = DNA
cgcacgagccgtgatctgttt

SEQ ID NO: 996 - DNA, 21 bp, synthetic construct
  source 1..21: mol_type = other DNA
  misc_feature 1..19: note = RNA
  misc_feature 20..21: note = DNA
cagatcacggctcgtgcgttt

SEQ ID NO: 997 - DNA, 21 bp, synthetic construct
  source 1..21: mol_type = other DNA
  misc_feature 1..19: note = RNA
  misc_feature 20..21: note = DNA
acgcacgagccgtgatctgtt

SEQ ID NO: 998 - DNA, 21 bp, synthetic construct
  source 1..21: mol_type = other DNA
  misc_feature 1..19: note = RNA
  misc_feature 20..21: note = DNA
agatcacggctcgtgcgtctt

SEQ ID NO: 999 - DNA, 21 bp, synthetic construct
  source 1..21: mol_type = other DNA
  misc_feature 1..19: note = RNA
  misc_feature 20..21: note = DNA
gacgcacgagccgtgatcttt

SEQ ID NO: 1000 - DNA, 21 bp, synthetic construct
  source 1..21: mol_type = other DNA
  misc_feature 1..19: note = RNA
  misc_feature 20..21: note = DNA
gatcacggctcgtgcgtcctt

SEQ ID NO: 1001 - DNA, 21 bp, synthetic construct
  source 1..21: mol_type = other DNA
  misc_feature 1..19: note = RNA
  misc_feature 20..21: note = DNA
ggacgcacgagccgtgatctt

SEQ ID NO: 1002 - DNA, 21 bp, synthetic construct
  source 1..21: mol_type = other DNA
  misc_feature 1..19: note = RNA
  misc_feature 20..21: note = DNA
atcacggctcgtgcgtccgtt

SEQ ID NO: 1003 - DNA, 21 bp, synthetic construct
  source 1..21: mol_type = other DNA
  misc_feature 1..19: note = RNA
  misc_feature 20..21: note = DNA
cggacgcacgagccgtgattt

SEQ ID NO: 1004 - DNA, 21 bp, synthetic construct
  source 1..21: mol_type = other DNA
  misc_feature 1..19: note = RNA
  misc_feature 20..21: note = DNA
tcacggctcgtgcgtccgatt

SEQ ID NO: 1005 - DNA, 21 bp, synthetic construct
  source 1..21: mol_type = other DNA
  misc_feature 1..19: note = RNA
  misc_feature 20..21: note = DNA
tcggacgcacgagccgtgatt

SEQ ID NO: 1006 - DNA, 21 bp, synthetic construct
  source 1..21: mol_type = other DNA
  misc_feature 1..19: note = RNA
  misc_feature 20..21: note = DNA
cacggctcgtgcgtccgagtt

SEQ ID NO: 1007 - DNA, 21 bp, synthetic construct
  source 1..21: mol_type = other DNA
  misc_feature 1..19: note = RNA
  misc_feature 20..21: note = DNA
ctcggacgcacgagccgtgtt

SEQ ID NO: 1008 - DNA, 21 bp, synthetic construct
  source 1..21: mol_type = other DNA
  misc_feature 1..19: note = RNA
  misc_feature 20..21: note = DNA
acggctcgtgcgtccgagctt

SEQ ID NO: 1009 - DNA, 21 bp, synthetic construct
  source 1..21: mol_type = other DNA
  misc_feature 1..19: note = RNA
  misc_feature 20..21: note = DNA
gctcggacgcacgagccgttt

SEQ ID NO: 1010 - DNA, 21 bp, synthetic construct
  source 1..21: mol_type = other DNA
  misc_feature 1..19: note = RNA
  misc_feature 20..21: note = DNA
gctcgtgcgtccgagcctgtt

SEQ ID NO: 1011 - DNA, 21 bp, synthetic construct
  source 1..21: mol_type = other DNA
  misc_feature 1..19: note = RNA
  misc_feature 20..21: note = DNA
caggctcggacgcacgagctt

SEQ ID NO: 1012 - DNA, 21 bp, synthetic construct
  source 1..21: mol_type = other DNA
  misc_feature 1..19: note = RNA
  misc_feature 20..21: note = DNA
ggaggaagacggcgtccgctt

SEQ ID NO: 1013 - DNA, 21 bp, synthetic construct
  source 1..21: mol_type = other DNA
  misc_feature 1..19: note = RNA
  misc_feature 20..21: note = DNA
gcggacgccgtcttcctcctt

SEQ ID NO: 1014 - DNA, 21 bp, synthetic construct
  source 1..21: mol_type = other DNA
  misc_feature 1..19: note = RNA
  misc_feature 20..21: note = DNA
gaggaagacggcgtccgcatt

SEQ ID NO: 1015 - DNA, 21 bp, synthetic construct
  source 1..21: mol_type = other DNA
  misc_feature 1..19: note = RNA
  misc_feature 20..21: note = DNA
tgcggacgccgtcttcctctt

SEQ ID NO: 1016 - DNA, 21 bp, synthetic construct
  source 1..21: mol_type = other DNA
  misc_feature 1..19: note = RNA
  misc_feature 20..21: note = DNA
ggaagacggcgtccgcaagtt

SEQ ID NO: 1017 - DNA, 21 bp, synthetic construct
  source 1..21: mol_type = other DNA
  misc_feature 1..19: note = RNA
  misc_feature 20..21: note = DNA
cttgcggacgccgtcttcctt

SEQ ID NO: 1018 - DNA, 21 bp, synthetic construct
  source 1..21: mol_type = other DNA
  misc_feature 1..19: note = RNA
  misc_feature 20..21: note = DNA
gaagacggcgtccgcaagttt

SEQ ID NO: 1019 - DNA, 21 bp, synthetic construct
  source 1..21: mol_type = other DNA
  misc_feature 1..19: note = RNA
  misc_feature 20..21: note = DNA
acttgcggacgccgtcttctt

SEQ ID NO: 1020 - DNA, 21 bp, synthetic construct
  source 1..21: mol_type = other DNA
  misc_feature 1..19: note = RNA
  misc_feature 20..21: note = DNA
aagacggcgtccgcaagtgtt

SEQ ID NO: 1021 - DNA, 21 bp, synthetic construct
  source 1..21: mol_type = other DNA
  misc_feature 1..19: note = RNA
  misc_feature 20..21: note = DNA
cacttgcggacgccgtctttt

SEQ ID NO: 1022 - DNA, 21 bp, synthetic construct
  source 1..21: mol_type = other DNA
  misc_feature 1..19: note = RNA
  misc_feature 20..21: note = DNA
gacggcgtccgcaagtgtatt

SEQ ID NO: 1023 - DNA, 21 bp, synthetic construct
  source 1..21: mol_type = other DNA
  misc_feature 1..19: note = RNA
  misc_feature 20..21: note = DNA
tacacttgcggacgccgtctt

SEQ ID NO: 1024 - DNA, 21 bp, synthetic construct
  source 1..21: mol_type = other DNA
  misc_feature 1..19: note = RNA
  misc_feature 20..21: note = DNA
acggcgtccgcaagtgtaatt

SEQ ID NO: 1025 - DNA, 21 bp, synthetic construct
  source 1..21: mol_type = other DNA
  misc_feature 1..19: note = RNA
  misc_feature 20..21: note = DNA
ttacacttgcggacgccgttt

SEQ ID NO: 1026 - DNA, 21 bp, synthetic construct
  source 1..21: mol_type = other DNA
  misc_feature 1..19: note = RNA
  misc_feature 20..21: note = DNA
gcgtccgcaagtgtaagaatt

SEQ ID NO: 1027 - DNA, 21 bp, synthetic construct
  source 1..21: mol_type = other DNA
  misc_feature 1..19: note = RNA
  misc_feature 20..21: note = DNA
ttcttacacttgcggacgctt

SEQ ID NO: 1028 - DNA, 21 bp, synthetic construct
  source 1..21: mol_type = other DNA
  misc_feature 1..19: note = RNA
  misc_feature 20..21: note = DNA
cgtccgcaagtgtaagaagtt

SEQ ID NO: 1029 - DNA, 21 bp, synthetic construct
  source 1..21: mol_type = other DNA
  misc_feature 1..19: note = RNA
  misc_feature 20..21: note = DNA
cttcttacacttgcggacgtt

SEQ ID NO: 1030 - DNA, 21 bp, synthetic construct
  source 1..21: mol_type = other DNA
  misc_feature 1..19: note = RNA
  misc_feature 20..21: note = DNA
gtccgcaagtgtaagaagttt

SEQ ID NO: 1031 - DNA, 21 bp, synthetic construct
  source 1..21: mol_type = other DNA
  misc_feature 1..19: note = RNA
  misc_feature 20..21: note = DNA
acttcttacacttgcggactt

SEQ ID NO: 1032 - DNA, 21 bp, synthetic construct
  source 1..21: mol_type = other DNA
  misc_feature 1..19: note = RNA
  misc_feature 20..21: note = DNA
tccgcaagtgtaagaagtgtt

SEQ ID NO: 1033 - DNA, 21 bp, synthetic construct
  source 1..21: mol_type = other DNA
  misc_feature 1..19: note = RNA
  misc_feature 20..21: note = DNA
cacttcttacacttgcggatt

SEQ ID NO: 1034 - DNA, 21 bp, synthetic construct
  source 1..21: mol_type = other DNA
  misc_feature 1..19: note = RNA
  misc_feature 20..21: note = DNA
ccgcaagtgtaagaagtgctt

SEQ ID NO: 1035 - DNA, 21 bp, synthetic construct
  source 1..21: mol_type = other DNA
  misc_feature 1..19: note = RNA
  misc_feature 20..21: note = DNA
gcacttcttacacttgcggtt

SEQ ID NO: 1036 - DNA, 21 bp, synthetic construct
  source 1..21: mol_type = other DNA
  misc_feature 1..19: note = RNA
  misc_feature 20..21: note = DNA
cgcaagtgtaagaagtgcgtt

SEQ ID NO: 1037 - DNA, 21 bp, synthetic construct
  source 1..21: mol_type = other DNA
  misc_feature 1..19: note = RNA
  misc_feature 20..21: note = DNA
cgcacttcttacacttgcgtt

SEQ ID NO: 1038 - DNA, 21 bp, synthetic construct
  source 1..21: mol_type = other DNA
  misc_feature 1..19: note = RNA
  misc_feature 20..21: note = DNA
gcaagtgtaagaagtgcgatt

SEQ ID NO: 1039 - DNA, 21 bp, synthetic construct
  source 1..21: mol_type = other DNA
  misc_feature 1..19: note = RNA
  misc_feature 20..21: note = DNA
tcgcacttcttacacttgctt

SEQ ID NO: 1040 - DNA, 21 bp, synthetic construct
  source 1..21: mol_type = other DNA
  misc_feature 1..19: note = RNA
  misc_feature 20..21: note = DNA
aagtgtaagaagtgcgaagtt

SEQ ID NO: 1041 - DNA, 21 bp, synthetic construct
  source 1..21: mol_type = other DNA
  misc_feature 1..19: note = RNA
  misc_feature 20..21: note = DNA
cttcgcacttcttacactttt

SEQ ID NO: 1042 - DNA, 21 bp, synthetic construct
  source 1..21: mol_type = other DNA
  misc_feature 1..19: note = RNA
  misc_feature 20..21: note = DNA
agtgtaagaagtgcgaaggtt

SEQ ID NO: 1043 - DNA, 21 bp, synthetic construct
  source 1..21: mol_type = other DNA
  misc_feature 1..19: note = RNA
  misc_feature 20..21: note = DNA
ccttcgcacttcttacacttt

SEQ ID NO: 1044 - DNA, 21 bp, synthetic construct
  source 1..21: mol_type = other DNA
  misc_feature 1..19: note = RNA
  misc_feature 20..21: note = DNA
gtgtaagaagtgcgaagggtt

SEQ ID NO: 1045 - DNA, 21 bp, synthetic construct
  source 1..21: mol_type = other DNA
  misc_feature 1..19: note = RNA
  misc_feature 20..21: note = DNA
cccttcgcacttcttacactt

SEQ ID NO: 1046 - DNA, 21 bp, synthetic construct
  source 1..21: mol_type = other DNA
  misc_feature 1..19: note = RNA
  misc_feature 20..21: note = DNA
tgtaagaagtgcgaagggctt

SEQ ID NO: 1047 - DNA, 21 bp, synthetic construct
  source 1..21: mol_type = other DNA
  misc_feature 1..19: note = RNA
  misc_feature 20..21: note = DNA
gcccttcgcacttcttacatt

SEQ ID NO: 1048 - DNA, 21 bp, synthetic construct
  source 1..21: mol_type = other DNA
  misc_feature 1..19: note = RNA
  misc_feature 20..21: note = DNA
gtaagaagtgcgaagggcctt

SEQ ID NO: 1049 - DNA, 21 bp, synthetic construct
  source 1..21: mol_type = other DNA
  misc_feature 1..19: note = RNA
  misc_feature 20..21: note = DNA
ggcccttcgcacttcttactt

SEQ ID NO: 1050 - DNA, 21 bp, synthetic construct
  source 1..21: mol_type = other DNA
  misc_feature 1..19: note = RNA
  misc_feature 20..21: note = DNA
taagaagtgcgaagggccttt

SEQ ID NO: 1051 - DNA, 21 bp, synthetic construct
  source 1..21: mol_type = other DNA
  misc_feature 1..19: note = RNA
  misc_feature 20..21: note = DNA
aggcccttcgcacttcttatt

SEQ ID NO: 1052 - DNA, 21 bp, synthetic construct
  source 1..21: mol_type = other DNA
  misc_feature 1..19: note = RNA
  misc_feature 20..21: note = DNA
aagaagtgcgaagggcctttt

SEQ ID NO: 1053 - DNA, 21 bp, synthetic construct
  source 1..21: mol_type = other DNA
  misc_feature 1..19: note = RNA
  misc_feature 20..21: note = DNA
aaggcccttcgcacttctttt

SEQ ID NO: 1054 - DNA, 21 bp, synthetic construct
  source 1..21: mol_type = other DNA
  misc_feature 1..19: note = RNA
  misc_feature 20..21: note = DNA
agaagtgcgaagggccttgtt

SEQ ID NO: 1055 - DNA, 21 bp, synthetic construct
  source 1..21: mol_type = other DNA
  misc_feature 1..19: note = RNA
  misc_feature 20..21: note = DNA
caaggcccttcgcacttcttt

SEQ ID NO: 1056 - DNA, 21 bp, synthetic construct
  source 1..21: mol_type = other DNA
  misc_feature 1..19: note = RNA
  misc_feature 20..21: note = DNA
agtgcgaagggccttgccgtt

SEQ ID NO: 1057 - DNA, 21 bp, synthetic construct
  source 1..21: mol_type = other DNA
  misc_feature 1..19: note = RNA
  misc_feature 20..21: note = DNA
cggcaaggcccttcgcacttt

SEQ ID NO: 1058 - DNA, 21 bp, synthetic construct
  source 1..21: mol_type = other DNA
  misc_feature 1..19: note = RNA
  misc_feature 20..21: note = DNA
gtgcgaagggccttgccgctt

SEQ ID NO: 1059 - DNA, 21 bp, synthetic construct
  source 1..21: mol_type = other DNA
  misc_feature 1..19: note = RNA
  misc_feature 20..21: note = DNA
gcggcaaggcccttcgcactt

SEQ ID NO: 1060 - DNA, 21 bp, synthetic construct
  source 1..21: mol_type = other DNA
  misc_feature 1..19: note = RNA
  misc_feature 20..21: note = DNA
tgcgaagggccttgccgcatt

SEQ ID NO: 1061 - DNA, 21 bp, synthetic construct
  source 1..21: mol_type = other DNA
  misc_feature 1..19: note = RNA
  misc_feature 20..21: note = DNA
tgcggcaaggcccttcgcatt

SEQ ID NO: 1062 - DNA, 21 bp, synthetic construct
  source 1..21: mol_type = other DNA
  misc_feature 1..19: note = RNA
  misc_feature 20..21: note = DNA
gcgaagggccttgccgcaatt

SEQ ID NO: 1063 - DNA, 21 bp, synthetic construct
  source 1..21: mol_type = other DNA
  misc_feature 1..19: note = RNA
  misc_feature 20..21: note = DNA
ttgcggcaaggcccttcgctt

SEQ ID NO: 1064 - DNA, 21 bp, synthetic construct
  source 1..21: mol_type = other DNA
  misc_feature 1..19: note = RNA
  misc_feature 20..21: note = DNA
cgaagggccttgccgcaaatt

SEQ ID NO: 1065 - DNA, 21 bp, synthetic construct
  source 1..21: mol_type = other DNA
  misc_feature 1..19: note = RNA
  misc_feature 20..21: note = DNA
tttgcggcaaggcccttcgtt

SEQ ID NO: 1066 - DNA, 21 bp, synthetic construct
  source 1..21: mol_type = other DNA
  misc_feature 1..19: note = RNA
  misc_feature 20..21: note = DNA
aagggccttgccgcaaagttt

SEQ ID NO: 1067 - DNA, 21 bp, synthetic construct
  source 1..21: mol_type = other DNA
  misc_feature 1..19: note = RNA
  misc_feature 20..21: note = DNA
actttgcggcaaggccctttt

SEQ ID NO: 1068 - DNA, 21 bp, synthetic construct
  source 1..21: mol_type = other DNA
  misc_feature 1..19: note = RNA
  misc_feature 20..21: note = DNA
agggccttgccgcaaagtgtt

SEQ ID NO: 1069 - DNA, 21 bp, synthetic construct
  source 1..21: mol_type = other DNA
  misc_feature 1..19: note = RNA
  misc_feature 20..21: note = DNA
cactttgcggcaaggcccttt

SEQ ID NO: 1070 - DNA, 21 bp, synthetic construct
  source 1..21: mol_type = other DNA
  misc_feature 1..19: note = RNA
  misc_feature 20..21: note = DNA
gggccttgccgcaaagtgttt

SEQ ID NO: 1071 - DNA, 21 bp, synthetic construct
  source 1..21: mol_type = other DNA
  misc_feature 1..19: note = RNA
  misc_feature 20..21: note = DNA
acactttgcggcaaggccctt

SEQ ID NO: 1072 - DNA, 21 bp, synthetic construct
  source 1..21: mol_type = other DNA
  misc_feature 1..19: note = RNA
  misc_feature 20..21: note = DNA
ggccttgccgcaaagtgtgtt

SEQ ID NO: 1073 - DNA, 21 bp, synthetic construct
  source 1..21: mol_type = other DNA
  misc_feature 1..19: note = RNA
  misc_feature 20..21: note = DNA
cacactttgcggcaaggcctt

SEQ ID NO: 1074 - DNA, 21 bp, synthetic construct
  source 1..21: mol_type = other DNA
  misc_feature 1..19: note = RNA
  misc_feature 20..21: note = DNA
gccttgccgcaaagtgtgttt

SEQ ID NO: 1075 - DNA, 21 bp, synthetic construct
  source 1..21: mol_type = other DNA
  misc_feature 1..19: note = RNA
  misc_feature 20..21: note = DNA
acacactttgcggcaaggctt

SEQ ID NO: 1076 - DNA, 21 bp, synthetic construct
  source 1..21: mol_type = other DNA
  misc_feature 1..19: note = RNA
  misc_feature 20..21: note = DNA
cttgccgcaaagtgtgtaatt

SEQ ID NO: 1077 - DNA, 21 bp, synthetic construct
  source 1..21: mol_type = other DNA
  misc_feature 1..19: note = RNA
  misc_feature 20..21: note = DNA
ttacacactttgcggcaagtt

SEQ ID NO: 1078 - DNA, 21 bp, synthetic construct
  source 1..21: mol_type = other DNA
  misc_feature 1..19: note = RNA
  misc_feature 20..21: note = DNA
ggaataggtattggtgaattt

SEQ ID NO: 1079 - DNA, 21 bp, synthetic construct
  source 1..21: mol_type = other DNA
  misc_feature 1..19: note = RNA
  misc_feature 20..21: note = DNA
attcaccaatacctattcctt

SEQ ID NO: 1080 - DNA, 21 bp, synthetic construct
  source 1..21: mol_type = other DNA
  misc_feature 1..19: note = RNA
  misc_feature 20..21: note = DNA
gaataggtattggtgaatttt

SEQ ID NO: 1081 - DNA, 21 bp, synthetic construct
  source 1..21: mol_type = other DNA
  misc_feature 1..19: note = RNA
  misc_feature 20..21: note = DNA
aattcaccaatacctattctt

SEQ ID NO: 1082 - DNA, 21 bp, synthetic construct
  source 1..21: mol_type = other DNA
  misc_feature 1..19: note = RNA
  misc_feature 20..21: note = DNA
aataggtattggtgaattttt

SEQ ID NO: 1083 - DNA, 21 bp, synthetic construct
  source 1..21: mol_type = other DNA
  misc_feature 1..19: note = RNA
  misc_feature 20..21: note = DNA
aaattcaccaatacctatttt

SEQ ID NO: 1084 - DNA, 21 bp, synthetic construct
  source 1..21: mol_type = other DNA
  misc_feature 1..19: note = RNA
  misc_feature 20..21: note = DNA
ataggtattggtgaatttatt

SEQ ID NO: 1085 - DNA, 21 bp, synthetic construct
  source 1..21: mol_type = other DNA
  misc_feature 1..19: note = RNA
  misc_feature 20..21: note = DNA
taaattcaccaatacctattt

SEQ ID NO: 1086 - DNA, 21 bp, synthetic construct
  source 1..21: mol_type = other DNA
  misc_feature 1..19: note = RNA
  misc_feature 20..21: note = DNA
aggtattggtgaatttaaatt

SEQ ID NO: 1087 - DNA, 21 bp, synthetic construct
  source 1..21: mol_type = other DNA
  misc_feature 1..19: note = RNA
  misc_feature 20..21: note = DNA
tttaaattcaccaataccttt

SEQ ID NO: 1088 - DNA, 21 bp, synthetic construct
  source 1..21: mol_type = other DNA
  misc_feature 1..19: note = RNA
  misc_feature 20..21: note = DNA
ggtattggtgaatttaaagtt

SEQ ID NO: 1089 - DNA, 21 bp, synthetic construct
  source 1..21: mol_type = other DNA
  misc_feature 1..19: note = RNA
  misc_feature 20..21: note = DNA
ctttaaattcaccaatacctt

SEQ ID NO: 1090 - DNA, 21 bp, synthetic construct
  source 1..21: mol_type = other DNA
  misc_feature 1..19: note = RNA
  misc_feature 20..21: note = DNA
cactctccataaatgctactt

SEQ ID NO: 1091 - DNA, 21 bp, synthetic construct
  source 1..21: mol_type = other DNA
  misc_feature 1..19: note = RNA
  misc_feature 20..21: note = DNA
gtagcatttatggagagtgtt

SEQ ID NO: 1092 - DNA, 21 bp, synthetic construct
  source 1..21: mol_type = other DNA
  misc_feature 1..19: note = RNA
  misc_feature 20..21: note = DNA
ttaaacacttcaaaaactgtt

SEQ ID NO: 1093 - DNA, 21 bp, synthetic construct
  source 1..21: mol_type = other DNA
  misc_feature 1..19: note = RNA
  misc_feature 20..21: note = DNA
cagtttttgaagtgtttaatt

SEQ ID NO: 1094 - DNA, 21 bp, synthetic construct
  source 1..21: mol_type = other DNA
  misc_feature 1..19: note = RNA
  misc_feature 20..21: note = DNA
cttcaaaaactgcacctcctt

SEQ ID NO: 1095 - DNA, 21 bp, synthetic construct
  source 1..21: mol_type = other DNA
  misc_feature 1..19: note = RNA
  misc_feature 20..21: note = DNA
ggaggtgcagtttttgaagtt

SEQ ID NO: 1096 - DNA, 21 bp, synthetic construct
  source 1..21: mol_type = other DNA
  misc_feature 1..19: note = RNA
  misc_feature 20..21: note = DNA
ttcaaaaactgcacctccatt

SEQ ID NO: 1097 - DNA, 21 bp, synthetic construct
  source 1..21: mol_type = other DNA
  misc_feature 1..19: note = RNA
  misc_feature 20..21: note = DNA
tggaggtgcagtttttgaatt

SEQ ID NO: 1098 - DNA, 21 bp, synthetic construct
  source 1..21: mol_type = other DNA
  misc_feature 1..19: note = RNA
  misc_feature 20..21: note = DNA
tcaaaaactgcacctccattt

SEQ ID NO: 1099 - DNA, 21 bp, synthetic construct
  source 1..21: mol_type = other DNA
  misc_feature 1..19: note = RNA
  misc_feature 20..21: note = DNA
atggaggtgcagtttttgatt

SEQ ID NO: 1100 - DNA, 21 bp, synthetic construct
  source 1..21: mol_type = other DNA
  misc_feature 1..19: note = RNA
  misc_feature 20..21: note = DNA
caaaaactgcacctccatctt

SEQ ID NO: 1101 - DNA, 21 bp, synthetic construct
  source 1..21: mol_type = other DNA
  misc_feature 1..19: note = RNA
  misc_feature 20..21: note = DNA
gatggaggtgcagtttttgtt

SEQ ID NO: 1102 - DNA, 21 bp, synthetic construct
  source 1..21: mol_type = other DNA
  misc_feature 1..19: note = RNA
  misc_feature 20..21: note = DNA
aaaaactgcacctccatcatt

SEQ ID NO: 1103 - DNA, 21 bp, synthetic construct
  source 1..21: mol_type = other DNA
  misc_feature 1..19: note = RNA
  misc_feature 20..21: note = DNA
tgatggaggtgcagttttttt

SEQ ID NO: 1104 - DNA, 21 bp, synthetic construct
  source 1..21: mol_type = other DNA
  misc_feature 1..19: note = RNA
  misc_feature 20..21: note = DNA
actgcacctccatcagtggtt

SEQ ID NO: 1105 - DNA, 21 bp, synthetic construct
  source 1..21: mol_type = other DNA
  misc_feature 1..19: note = RNA
  misc_feature 20..21: note = DNA
ccactgatggaggtgcagttt

SEQ ID NO: 1106 - DNA, 21 bp, synthetic construct
  source 1..21: mol_type = other DNA
  misc_feature 1..19: note = RNA
  misc_feature 20..21: note = DNA
cacctccatcagtggcgattt

SEQ ID NO: 1107 - DNA, 21 bp, synthetic construct
  source 1..21: mol_type = other DNA
  misc_feature 1..19: note = RNA
  misc_feature 20..21: note = DNA
atcgccactgatggaggtgtt

SEQ ID NO: 1108 - DNA, 21 bp, synthetic construct
  source 1..21: mol_type = other DNA
  misc_feature 1..19: note = RNA
  misc_feature 20..21: note = DNA
acctccatcagtggcgatctt

SEQ ID NO: 1109 - DNA, 21 bp, synthetic construct
  source 1..21: mol_type = other DNA
  misc_feature 1..19: note = RNA
  misc_feature 20..21: note = DNA
gatcgccactgatggaggttt

SEQ ID NO: 1110 - DNA, 21 bp, synthetic construct
  source 1..21: mol_type = other DNA
  misc_feature 1..19: note = RNA
  misc_feature 20..21: note = DNA
ctccatcagtggcgatctctt

SEQ ID NO: 1111 - DNA, 21 bp, synthetic construct
  source 1..21: mol_type = other DNA
  misc_feature 1..19: note = RNA
  misc_feature 20..21: note = DNA
gagatcgccactgatggagtt

SEQ ID NO: 1112 - DNA, 21 bp, synthetic construct
  source 1..21: mol_type = other DNA
  misc_feature 1..19: note = RNA
  misc_feature 20..21: note = DNA
catcagtggcgatctccactt

SEQ ID NO: 1113 - DNA, 21 bp, synthetic construct
  source 1..21: mol_type = other DNA
  misc_feature 1..19: note = RNA
  misc_feature 20..21: note = DNA
gtggagatcgccactgatgtt

SEQ ID NO: 1114 - DNA, 21 bp, synthetic construct
  source 1..21: mol_type = other DNA
  misc_feature 1..19: note = RNA
  misc_feature 20..21: note = DNA
tggcgatctccacatcctgtt

SEQ ID NO: 1115 - DNA, 21 bp, synthetic construct
  source 1..21: mol_type = other DNA
  misc_feature 1..19: note = RNA
  misc_feature 20..21: note = DNA
caggatgtggagatcgccatt

SEQ ID NO: 1116 - DNA, 21 bp, synthetic construct
  source 1..21: mol_type = other DNA
  misc_feature 1..19: note = RNA
  misc_feature 20..21: note = DNA
ggcgatctccacatcctgctt

SEQ ID NO: 1117 - DNA, 21 bp, synthetic construct
  source 1..21: mol_type = other DNA
  misc_feature 1..19: note = RNA
  misc_feature 20..21: note = DNA
gcaggatgtggagatcgcctt

SEQ ID NO: 1118 - DNA, 21 bp, synthetic construct
  source 1..21: mol_type = other DNA
  misc_feature 1..19: note = RNA
  misc_feature 20..21: note = DNA
gcgatctccacatcctgcctt

SEQ ID NO: 1119 - DNA, 21 bp, synthetic construct
  source 1..21: mol_type = other DNA
  misc_feature 1..19: note = RNA
  misc_feature 20..21: note = DNA
ggcaggatgtggagatcgctt

SEQ ID NO: 1120 - DNA, 21 bp, synthetic construct
  source 1..21: mol_type = other DNA
  misc_feature 1..19: note = RNA
  misc_feature 20..21: note = DNA
cgatctccacatcctgccgtt

SEQ ID NO: 1121 - DNA, 21 bp, synthetic construct
  source 1..21: mol_type = other DNA
  misc_feature 1..19: note = RNA
  misc_feature 20..21: note = DNA
cggcaggatgtggagatcgtt

SEQ ID NO: 1122 - DNA, 21 bp, synthetic construct
  source 1..21: mol_type = other DNA
  misc_feature 1..19: note = RNA
  misc_feature 20..21: note = DNA
gatctccacatcctgccggtt

SEQ ID NO: 1123 - DNA, 21 bp, synthetic construct
  source 1..21: mol_type = other DNA
  misc_feature 1..19: note = RNA
  misc_feature 20..21: note = DNA
ccggcaggatgtggagatctt

SEQ ID NO: 1124 - DNA, 21 bp, synthetic construct
  source 1..21: mol_type = other DNA
  misc_feature 1..19: note = RNA
  misc_feature 20..21: note = DNA
ccacatcctgccggtggcatt

SEQ ID NO: 1125 - DNA, 21 bp, synthetic construct
  source 1..21: mol_type = other DNA
  misc_feature 1..19: note = RNA
  misc_feature 20..21: note = DNA
tgccaccggcaggatgtggtt

SEQ ID NO: 1126 - DNA, 21 bp, synthetic construct
  source 1..21: mol_type = other DNA
  misc_feature 1..19: note = RNA
  misc_feature 20..21: note = DNA
cacatcctgccggtggcattt

SEQ ID NO: 1127 - DNA, 21 bp, synthetic construct
  source 1..21: mol_type = other DNA
  misc_feature 1..19: note = RNA
  misc_feature 20..21: note = DNA
atgccaccggcaggatgtgtt

SEQ ID NO: 1128 - DNA, 21 bp, synthetic construct
  source 1..21: mol_type = other DNA
  misc_feature 1..19: note = RNA
  misc_feature 20..21: note = DNA
acatcctgccggtggcatttt

SEQ ID NO: 1129 - DNA, 21 bp, synthetic construct
  source 1..21: mol_type = other DNA
  misc_feature 1..19: note = RNA
  misc_feature 20..21: note = DNA
aatgccaccggcaggatgttt

SEQ ID NO: 1130 - DNA, 21 bp, synthetic construct
  source 1..21: mol_type = other DNA
  misc_feature 1..19: note = RNA
  misc_feature 20..21: note = DNA
atcctgccggtggcatttatt

SEQ ID NO: 1131 - DNA, 21 bp, synthetic construct
  source 1..21: mol_type = other DNA
  misc_feature 1..19: note = RNA
  misc_feature 20..21: note = DNA
taaatgccaccggcaggattt

SEQ ID NO: 1132 - DNA, 21 bp, synthetic construct
  source 1..21: mol_type = other DNA
  misc_feature 1..19: note = RNA
  misc_feature 20..21: note = DNA
ctgccggtggcatttagggtt

SEQ ID NO: 1133 - DNA, 21 bp, synthetic construct
  source 1..21: mol_type = other DNA
  misc_feature 1..19: note = RNA
  misc_feature 20..21: note = DNA
ccctaaatgccaccggcagtt

SEQ ID NO: 1134 - DNA, 21 bp, synthetic construct
  source 1..21: mol_type = other DNA
  misc_feature 1..19: note = RNA
  misc_feature 20..21: note = DNA
tgccggtggcatttaggggtt

SEQ ID NO: 1135 - DNA, 21 bp, synthetic construct
  source 1..21: mol_type = other DNA
  misc_feature 1..19: note = RNA
  misc_feature 20..21: note = DNA
cccctaaatgccaccggcatt

SEQ ID NO: 1136 - DNA, 21 bp, synthetic construct
  source 1..21: mol_type = other DNA
  misc_feature 1..19: note = RNA
  misc_feature 20..21: note = DNA
gccggtggcatttaggggttt

SEQ ID NO: 1137 - DNA, 21 bp, synthetic construct
  source 1..21: mol_type = other DNA
  misc_feature 1..19: note = RNA
  misc_feature 20..21: note = DNA
acccctaaatgccaccggctt

SEQ ID NO: 1138 - DNA, 21 bp, synthetic construct
  source 1..21: mol_type = other DNA
  misc_feature 1..19: note = RNA
  misc_feature 20..21: note = DNA
ccggtggcatttaggggtgtt

SEQ ID NO: 1139 - DNA, 21 bp, synthetic construct
  source 1..21: mol_type = other DNA
  misc_feature 1..19: note = RNA
  misc_feature 20..21: note = DNA
cacccctaaatgccaccggtt

SEQ ID NO: 1140 - DNA, 21 bp, synthetic construct
  source 1..21: mol_type = other DNA
  misc_feature 1..19: note = RNA
  misc_feature 20..21: note = DNA
gtggcatttaggggtgacttt

SEQ ID NO: 1141 - DNA, 21 bp, synthetic construct
  source 1..21: mol_type = other DNA
  misc_feature 1..19: note = RNA
  misc_feature 20..21: note = DNA
agtcacccctaaatgccactt

SEQ ID NO: 1142 - DNA, 21 bp, synthetic construct
  source 1..21: mol_type = other DNA
  misc_feature 1..19: note = RNA
  misc_feature 20..21: note = DNA
gcatttaggggtgactccttt

SEQ ID NO: 1143 - DNA, 21 bp, synthetic construct
  source 1..21: mol_type = other DNA
  misc_feature 1..19: note = RNA
  misc_feature 20..21: note = DNA
aggagtcacccctaaatgctt

SEQ ID NO: 1144 - DNA, 21 bp, synthetic construct
  source 1..21: mol_type = other DNA
  misc_feature 1..19: note = RNA
  misc_feature 20..21: note = DNA
catttaggggtgactcctttt

SEQ ID NO: 1145 - DNA, 21 bp, synthetic construct
  source 1..21: mol_type = other DNA
  misc_feature 1..19: note = RNA
  misc_feature 20..21: note = DNA
aaggagtcacccctaaatgtt

SEQ ID NO: 1146 - DNA, 21 bp, synthetic construct
  source 1..21: mol_type = other DNA
  misc_feature 1..19: note = RNA
  misc_feature 20..21: note = DNA
atttaggggtgactccttctt

SEQ ID NO: 1147 - DNA, 21 bp, synthetic construct
  source 1..21: mol_type = other DNA
  misc_feature 1..19: note = RNA
  misc_feature 20..21: note = DNA
gaaggagtcacccctaaattt

SEQ ID NO: 1148 - DNA, 21 bp, synthetic construct
  source 1..21: mol_type = other DNA
  misc_feature 1..19: note = RNA
  misc_feature 20..21: note = DNA
tttaggggtgactccttcatt

SEQ ID NO: 1149 - DNA, 21 bp, synthetic construct
  source 1..21: mol_type = other DNA
  misc_feature 1..19: note = RNA
  misc_feature 20..21: note = DNA
tgaaggagtcacccctaaatt

SEQ ID NO: 1150 - DNA, 21 bp, synthetic construct
  source 1..21: mol_type = other DNA
  misc_feature 1..19: note = RNA
  misc_feature 20..21: note = DNA
ttaggggtgactccttcactt

SEQ ID NO: 1151 - DNA, 21 bp, synthetic construct
  source 1..21: mol_type = other DNA
  misc_feature 1..19: note = RNA
  misc_feature 20..21: note = DNA
gtgaaggagtcacccctaatt

SEQ ID NO: 1152 - DNA, 21 bp, synthetic construct
  source 1..21: mol_type = other DNA
  misc_feature 1..19: note = RNA
  misc_feature 20..21: note = DNA
taggggtgactccttcacatt

SEQ ID NO: 1153 - DNA, 21 bp, synthetic construct
  source 1..21: mol_type = other DNA
  misc_feature 1..19: note = RNA
  misc_feature 20..21: note = DNA
tgtgaaggagtcacccctatt

SEQ ID NO: 1154 - DNA, 21 bp, synthetic construct
  source 1..21: mol_type = other DNA
  misc_feature 1..19: note = RNA
  misc_feature 20..21: note = DNA
tctggatccacaggaactgtt

SEQ ID NO: 1155 - DNA, 21 bp, synthetic construct
  source 1..21: mol_type = other DNA
  misc_feature 1..19: note = RNA
  misc_feature 20..21: note = DNA
cagttcctgtggatccagatt

SEQ ID NO: 1156 - DNA, 21 bp, synthetic construct
  source 1..21: mol_type = other DNA
  misc_feature 1..19: note = RNA
  misc_feature 20..21: note = DNA
gatccacaggaactggatatt

SEQ ID NO: 1157 - DNA, 21 bp, synthetic construct
  source 1..21: mol_type = other DNA
  misc_feature 1..19: note = RNA
  misc_feature 20..21: note = DNA
tatccagttcctgtggatctt

SEQ ID NO: 1158 - DNA, 21 bp, synthetic construct
  source 1..21: mol_type = other DNA
  misc_feature 1..19: note = RNA
  misc_feature 20..21: note = DNA
atccacaggaactggatattt

SEQ ID NO: 1159 - DNA, 21 bp, synthetic construct
  source 1..21: mol_type = other DNA
  misc_feature 1..19: note = RNA
  misc_feature 20..21: note = DNA
atatccagttcctgtggattt

SEQ ID NO: 1160 - DNA, 21 bp, synthetic construct
  source 1..21: mol_type = other DNA
  misc_feature 1..19: note = RNA
  misc_feature 20..21: note = DNA
ccacaggaactggatattctt

SEQ ID NO: 1161 - DNA, 21 bp, synthetic construct
  source 1..21: mol_type = other DNA
  misc_feature 1..19: note = RNA
  misc_feature 20..21: note = DNA
gaatatccagttcctgtggtt

SEQ ID NO: 1162 - DNA, 21 bp, synthetic construct
  source 1..21: mol_type = other DNA
  misc_feature 1..19: note = RNA
  misc_feature 20..21: note = DNA
cacaggaactggatattcttt

SEQ ID NO: 1163 - DNA, 21 bp, synthetic construct
  source 1..21: mol_type = other DNA
  misc_feature 1..19: note = RNA
  misc_feature 20..21: note = DNA
agaatatccagttcctgtgtt

SEQ ID NO: 1164 - DNA, 21 bp, synthetic construct
  source 1..21: mol_type = other DNA
  misc_feature 1..19: note = RNA
  misc_feature 20..21: note = DNA
actggatattctgaaaacctt

SEQ ID NO: 1165 - DNA, 21 bp, synthetic construct
  source 1..21: mol_type = other DNA
  misc_feature 1..19: note = RNA
  misc_feature 20..21: note = DNA
ggttttcagaatatccagttt

SEQ ID NO: 1166 - DNA, 21 bp, synthetic construct
  source 1..21: mol_type = other DNA
  misc_feature 1..19: note = RNA
  misc_feature 20..21: note = DNA
attctgaaaaccgtaaaggtt

SEQ ID NO: 1167 - DNA, 21 bp, synthetic construct
  source 1..21: mol_type = other DNA
  misc_feature 1..19: note = RNA
  misc_feature 20..21: note = DNA
cctttacggttttcagaattt

SEQ ID NO: 1168 - DNA, 21 bp, synthetic construct
  source 1..21: mol_type = other DNA
  misc_feature 1..19: note = RNA
  misc_feature 20..21: note = DNA
ttctgaaaaccgtaaaggatt

SEQ ID NO: 1169 - DNA, 21 bp, synthetic construct
  source 1..21: mol_type = other DNA
  misc_feature 1..19: note = RNA
  misc_feature 20..21: note = DNA
tcctttacggttttcagaatt

SEQ ID NO: 1170 - DNA, 21 bp, synthetic construct
  source 1..21: mol_type = other DNA
  misc_feature 1..19: note = RNA
  misc_feature 20..21: note = DNA
tgaaaaccgtaaaggaaattt

SEQ ID NO: 1171 - DNA, 21 bp, synthetic construct
  source 1..21: mol_type = other DNA
  misc_feature 1..19: note = RNA
  misc_feature 20..21: note = DNA
atttcctttacggttttcatt

SEQ ID NO: 1172 - DNA, 21 bp, synthetic construct
  source 1..21: mol_type = other DNA
  misc_feature 1..19: note = RNA
  misc_feature 20..21: note = DNA
gaaaaccgtaaaggaaatctt

SEQ ID NO: 1173 - DNA, 21 bp, synthetic construct
  source 1..21: mol_type = other DNA
  misc_feature 1..19: note = RNA
  misc_feature 20..21: note = DNA
gatttcctttacggttttctt

SEQ ID NO: 1174 - RNA, 23 nt, Homo sapiens
  source 1..23: mol_type = genomic RNA
caaaggttctctgctagacgaca

SEQ ID NO: 1175 - RNA, 23 nt, Homo sapiens
  source 1..23: mol_type = genomic RNA
tctgggtgtcactatggagctct

SEQ ID NO: 1176 - RNA, 23 nt, Homo sapiens
  source 1..23: mol_type = genomic RNA
ctgggtgtcactatggagctctc

SEQ ID NO: 1177 - RNA, 23 nt, Homo sapiens
  source 1..23: mol_type = genomic RNA
gggtgtcactatggagctctcac

SEQ ID NO: 1178 - RNA, 23 nt, Homo sapiens
  source 1..23: mol_type = genomic RNA
tactacaactttccactggctct

SEQ ID NO: 1179 - RNA, 23 nt, unidentified
  source 1..23: mol_type = unassigned RNA; note = XD-01825K1 Human or Mouse target sequence
aagcttctgggtgtcactatgga

SEQ ID NO: 1180 - RNA, 23 nt, Homo sapiens
  source 1..23: mol_type = genomic RNA
cttctgggtgtcactatggagct

SEQ ID NO: 1181 - RNA, 21 nt, unidentified
  source 1..21: mol_type = unassigned RNA; note = Beta-catenin target sequence
ctgttggattgattcgaaatt

SEQ ID NO: 1182 - RNA, 21 nt, unidentified
  source 1..21: mol_type = unassigned RNA; note = Beta-catenin target sequence
tttcgaatcaatccaacagtt

SEQ ID NO: 1183 - RNA, 21 nt, unidentified
  source 1..21: mol_type = unassigned RNA; note = Beta-catenin target sequence
acgactagttcagttgctttt

SEQ ID NO: 1184 - RNA, 21 nt, unidentified
  source 1..21: mol_type = unassigned RNA; note = Beta-catenin target sequence
aagcaactgaactagtcgttt

SEQ ID NO: 1185 - DNA, 97 bp, Homo sapiens
  source 1..97: mol_type = genomic DNA
tgctgttgacagtgagcgccagctcaaagcaatttctacatagtgaagccacagatgtatgtagaaattgctttgagctgttgcctactgcctcgga

SEQ ID NO: 1186 - DNA, 97 bp, Homo sapiens
  source 1..97: mol_type = genomic DNA
tgctgttgacagtgagcgaaaggatgaaacacaaaaggtatagtgaagccacagatgtataccttttgtgtttcatccttctgcctactgcctcgga

SEQ ID NO: 1187 - DNA, 97 bp, Homo sapiens
  source 1..97: mol_type = genomic DNA
tgctgttgacagtgagcgccatgtcagagttactgtttcatagtgaagccacagatgtatgaaacagtaactctgacatgatgcctactgcctcgga

SEQ ID NO: 1188 - DNA, 97 bp, Homo sapiens
  source 1..97: mol_type = genomic DNA
tgctgttgacagtgagcgcaactagttcatttcaaaattatagtgaagccacagatgtataattttgaaatgaactagttttgcctactgcctcgga

SEQ ID NO: 1189 - DNA, 97 bp, Homo sapiens
  source 1..97: mol_type = genomic DNA
tgctgttgacagtgagcgcacagcaagaacagaaataaaatagtgaagccacagatgtattttatttctgttcttgctgtatgcctactgcctcgga

SEQ ID NO: 1190 - DNA, 97 bp, Homo sapiens
  source 1..97: mol_type = genomic DNA
tgctgttgacagtgagcgacaagatcaagaaaatgtatgatagtgaagccacagatgtatcatacattttcttgatcttgctgcctactgcctcgga

SEQ ID NO: 1191 - DNA, 97 bp, Homo sapiens
  source 1..97: mol_type = genomic DNA
tgctgttgacagtgagcgcagcaagttcacaattacccaatagtgaagccacagatgtattgggtaattgtgaacttgctttgcctactgcctcgga

SEQ ID NO: 1192 - DNA, 97 bp, Homo sapiens
  source 1..97: mol_type = genomic DNA
tgctgttgacagtgagcgccccttcgataagattattgaatagtgaagccacagatgtattcaataatcttatcgaagggatgcctactgcctcgga

SEQ ID NO: 1193 - DNA, 97 bp, Homo sapiens
  source 1..97: mol_type = genomic DNA
tgctgttgacagtgagcgagagcttgaagatgaaacacgatagtgaagccacagatgtatcgtgtttcatcttcaagctcctgcctactgcctcgga

SEQ ID NO: 1194 - DNA, 97 bp, Homo sapiens
  source 1..97: mol_type = genomic DNA
tgctgttgacagtgagcgacaccaaagaaaacacgaattatagtgaagccacagatgtataattcgtgttttctttggtggtgcctactgcctcgga

SEQ ID NO: 1195 - RNA, 22 nt, synthetic construct
  source 1..22: mol_type = other RNA
tgtagaaattgctttgagctgt

SEQ ID NO: 1196 - RNA, 22 nt, synthetic construct
  source 1..22: mol_type = other RNA
agctcaaagcaatttctacata

SEQ ID NO: 1197 - RNA, 22 nt, synthetic construct
  source 1..22: mol_type = other RNA
taccttttgtgtttcatccttc

SEQ ID NO: 1198 - RNA, 22 nt, synthetic construct
  source 1..22: mol_type = other RNA
aggatgaaacacaaaaggtata

SEQ ID NO: 1199 - RNA, 22 nt, synthetic construct
  source 1..22: mol_type = other RNA
tgaaacagtaactctgacatga

SEQ ID NO: 1200 - RNA, 22 nt, synthetic construct
  source 1..22: mol_type = other RNA
atgtcagagttactgtttcata

SEQ ID NO: 1201 - RNA, 22 nt, synthetic construct
  source 1..22: mol_type = other RNA
taattttgaaatgaactagttt

SEQ ID NO: 1202 - RNA, 22 nt, synthetic construct
  source 1..22: mol_type = other RNA
actagttcatttcaaaattata

SEQ ID NO: 1203 - RNA, 22 nt, synthetic construct
  source 1..22: mol_type = other RNA
ttttatttctgttcttgctgta

SEQ ID NO: 1204 - RNA, 22 nt, synthetic construct
  source 1..22: mol_type = other RNA
cagcaagaacagaaataaaata

SEQ ID NO: 1205 - RNA, 22 nt, synthetic construct
  source 1..22: mol_type = other RNA
tcatacattttcttgatcttgc

SEQ ID NO: 1206 - RNA, 22 nt, synthetic construct
  source 1..22: mol_type = other RNA
aagatcaagaaaatgtatgata

SEQ ID NO: 1207 - RNA, 22 nt, synthetic construct
  source 1..22: mol_type = other RNA
ttgggtaattgtgaacttgctt

SEQ ID NO: 1208 - RNA, 22 nt, synthetic construct
  source 1..22: mol_type = other RNA
gcaagttcacaattacccaata

SEQ ID NO: 1209 - RNA, 22 nt, synthetic construct
  source 1..22: mol_type = other RNA
ttcaataatcttatcgaaggga

SEQ ID NO: 1210 - RNA, 22 nt, synthetic construct
  source 1..22: mol_type = other RNA
ccttcgataagattattgaata

SEQ ID NO: 1211 - RNA, 22 nt, synthetic construct
  source 1..22: mol_type = other RNA
tcgtgtttcatcttcaagctcc

SEQ ID NO: 1212 - RNA, 22 nt, synthetic construct
  source 1..22: mol_type = other RNA
agcttgaagatgaaacacgata

SEQ ID NO: 1213 - RNA, 22 nt, synthetic construct
  source 1..22: mol_type = other RNA
taattcgtgttttctttggtgg

SEQ ID NO: 1214 - RNA, 22 nt, synthetic construct
  source 1..22: mol_type = other RNA
accaaagaaaacacgaattata

SEQ ID NO: 1215 - RNA, 21 nt, synthetic construct
  source 1..21: mol_type = other RNA
actcgtgccttggcaaacttt

SEQ ID NO: 1216 - RNA, 21 nt, synthetic construct
  source 1..21: mol_type = other RNA
agtttgccaaggcacgagttt

SEQ ID NO: 1217 - RNA, 21 nt, synthetic construct
  source 1..21: mol_type = other RNA
actcgtgccttggcaaacttt

SEQ ID NO: 1218 - RNA, 21 nt, synthetic construct
  source 1..21: mol_type = other RNA
agtttgccaaggcacgagttt

SEQ ID NO: 1219 - RNA, 21 nt, synthetic construct
  source 1..21: mol_type = other RNA
actcgtgccttggcaaacttt

SEQ ID NO: 1220 - RNA, 21 nt, synthetic construct
  source 1..21: mol_type = other RNA
agtttgccaaggcacgagttt

SEQ ID NO: 1221 - RNA, 21 nt, synthetic construct
  source 1..21: mol_type = other RNA
tgaattagctgtatcgtcatt

SEQ ID NO: 1222 - DNA, 21 bp, synthetic construct
  source 1..21: mol_type = other DNA
  misc_feature 1..19: note = RNA
  misc_feature 20..21: note = DNA
tgacgatacagctaattcatt

SEQ ID NO: 1223 - RNA, 21 nt, synthetic construct
  source 1..21: mol_type = other RNA
tgaattagctgtatcgtcatt

SEQ ID NO: 1224 - RNA, 21 nt, synthetic construct
  source 1..21: mol_type = other RNA
tgacgatacagctaattcatt

SEQ ID NO: 1225 - RNA, 21 nt, synthetic construct
  source 1..21: mol_type = other RNA
tgaattagctgtatcgtcatt

SEQ ID NO: 1226 - RNA, 21 nt, synthetic construct
  source 1..21: mol_type = other RNA
tgacgatacagctaattcatt

SEQ ID NO: 1227 - RNA, 21 nt, synthetic construct
  source 1..21: mol_type = other RNA
taagtataggtcctcattatt

SEQ ID NO: 1228 - RNA, 21 nt, synthetic construct
  source 1..21: mol_type = other RNA
taatgaggacctatacttatt

SEQ ID NO: 1229 - DNA, 21 bp, synthetic construct
  source 1..21: mol_type = other DNA
  misc_feature 1..19: note = RNA
  misc_feature 20..21: note = DNA
tttcgaatcaatccaacagtt

SEQ ID NO: 1230 - RNA, 21 nt, synthetic construct
  source 1..21: mol_type = other RNA
ctgttggattgattcgaaatt

SEQ ID NO: 1231 - RNA, 21 nt, synthetic construct
  source 1..21: mol_type = other RNA
tttcgaatcaatccaacagtt

SEQ ID NO: 1232 - RNA, 21 nt, synthetic construct
  source 1..21: mol_type = other RNA
ctgttggattgattcgaaatt

SEQ ID NO: 1233 - RNA, 21 nt, synthetic construct
  source 1..21: mol_type = other RNA
tttcgaatcaatccaacagtt

SEQ ID NO: 1234 - RNA, 21 nt, synthetic construct
  source 1..21: mol_type = other RNA
ctgttggattgattcgaaatt

SEQ ID NO: 1235 - RNA, 21 nt, synthetic construct
  source 1..21: mol_type = other RNA
ataaaatctacagtcatagtt

SEQ ID NO: 1236 - RNA, 21 nt, synthetic construct
  source 1..21: mol_type = other RNA
ctatgactgtagattttattt

SEQ ID NO: 1237 - RNA, 21 nt, synthetic construct
  source 1..21: mol_type = other RNA
ttaaaatctacagtcatagtt

SEQ ID NO: 1238 - RNA, 21 nt, synthetic construct
  source 1..21: mol_type = other RNA
ctatgactgtagattttaatt

SEQ ID NO: 1239 - RNA, 21 nt, synthetic construct
  source 1..21: mol_type = other RNA
ttaaaatctacagtcatagtt

SEQ ID NO: 1240 - RNA, 21 nt, synthetic construct
  source 1..21: mol_type = other RNA
ctatgactgtagattttaatt

SEQ ID NO: 1241 - RNA, 21 nt, synthetic construct
  source 1..21: mol_type = other RNA
gagagctccatagtgacactt

SEQ ID NO: 1242 - RNA, 21 nt, synthetic construct
  source 1..21: mol_type = other RNA
gtgtcactatggagctctctt

SEQ ID NO: 1243 - protein, 24 aa, synthetic construct
CGIFGEIEELIEEGLENLIDWGNA

SEQ ID NO: 1244 - protein, 24 aa, synthetic construct
GLFEAIEGFIENGWEGMIDGWYGC

SEQ ID NO: 1245 - protein, 27 aa, synthetic construct
GLFEAIEGFIENGWEGMIWDYGSGSCG

SEQ ID NO: 1246 - protein, 23 aa, synthetic construct
GLFEAIEGFIENGWEGMIDGWYG

SEQ ID NO: 1247 - protein, 27 aa, synthetic construct
GLFEAIEGFIENGWEGMIWDYGSGSCK

SEQ ID NO: 1248 - protein, 27 aa, synthetic construct
CLIGAILKVLATGLPTLISWIKNKRKQ

SEQ ID NO: 1249 - protein, 26 aa, synthetic construct
GIGAVLKVLTTGLPALISWIKRKRQQ

SEQ ID NO: 1250 - protein, 13 aa, synthetic construct
IFGAIAGLLKNIF

SEQ ID NO: 1251 - protein, 18 aa, synthetic construct
FFGHLFKLATKIIPSLFQ

SEQ ID NO: 1252 - protein, 21 aa, synthetic construct
KETWWETWWTEWSQPKKKRKV

SEQ ID NO: 1253 - protein, 19 aa, synthetic construct
LLIILRRRRIRKQAHAHSK

SEQ ID NO: 1254 - protein, 26 aa, synthetic construct
DPKGDPKGVTVTVTVTVTGKGDPKPD

SEQ ID NO: 1255 - protein, 17 aa, synthetic construct
CSIPPEVKFNKPFVYLI

SEQ ID NO: 1256 - protein, 27 aa, synthetic construct
GWTLNSAGYLLGYINLKALAALAKKIL

SEQ ID NO: 1257 - protein, 21 aa, synthetic construct
AGYLLGKINLKALAALAKKIL

SEQ ID NO: 1258 - protein, 17 aa, synthetic construct
GALFLGFLGAAGSTMGA

SEQ ID NO: 1259 - protein, 22 aa, synthetic construct
HGLASTLTHRRWAHYNALIRAF

SEQ ID NO: 1260 - protein, 20 aa, synthetic construct
GLWRALWRLLRSLWRLLWRA

SEQ ID NO: 1261 - protein, 30 aa, synthetic construct
WEAALAEALAEALAEHLAEALAEALEALAA

SEQ ID NO: 1262 - protein, 24 aa, synthetic construct
GLFEAIEGFIENGWEGMIDGWYGC

SEQ ID NO: 1263 - protein, 23 aa, synthetic construct
GLFGAIAGFIENGWEGMIDGWYG

SEQ ID NO: 1264 - protein, 36 aa, synthetic construct
GLFGAIAGFIENGWEGMIDGRQIKIWFQNRRMKWKK

SEQ ID NO: 1265 - protein, 26 aa, synthetic construct
GLFGAIAGFIENGWEGMIDGSSKKKK

SEQ ID NO: 1266 - protein, 24 aa, synthetic construct
GLFEAIAGFIENGWEGMIDGGGYC

SEQ ID NO: 1267 - protein, 23 aa, synthetic construct
GLFHAIAHFIHGGWHGLIHGWYG

SEQ ID NO: 1268 - protein, 30 aa, synthetic construct
GLFEAIEGFIENGWEGLAEALAEALEALAA

SEQ ID NO: 1269 - protein, 29 aa, synthetic construct
KWKLFKKIGAVLKVLTTGYGRKKRRQRRR

SEQ ID NO: 1270 - protein, 4 aa, synthetic construct
GGFG

SEQ ID NO: 1271 - protein, 4 aa, synthetic construct
ALAL

SEQ ID NO: 1272 - protein, 4 aa, synthetic construct
GFLG

SEQ ID NO: 1273 - RNA, 21 nt, synthetic construct
  source 1..21: mol_type = other RNA
ttattatttgttctttgcctt

SEQ ID NO: 1274 - RNA, 21 nt, synthetic construct
  source 1..21: mol_type = other RNA
ttacattaaagtctgttgttt

SEQ ID NO: 1275 - RNA, 21 nt, synthetic construct
  source 1..21: mol_type = other RNA
ttaaaatctacagtcatagtt

SEQ ID NO: 1276 - protein, 4 aa, synthetic construct
EAFQ

SEQ ID NO: 1277 - protein, 24 aa, synthetic construct
CGIFGEIEELIEEGLENLIDWGNG